(19) United States
(12) Patent Application Publication
(10) Pub. No.: US 2009/0291437 A1
(43) Pub. Date: Nov. 26, 2009

(54) METHODS FOR TARGETING QUADRUPLEX SEQUENCES

(76) Inventors: Sean O'Brien, Carlsbad (CA); Adam Siddiqui-Jain, San Diego, CA (US)

Correspondence Address: MORRISON & FOERSTER LLP, 12531 HIGH BLUFF DRIVE, SUITE 100, SAN DIEGO, CA, 92130-2040, US

(21) Appl. No.: 12/092,557

(22) PCT Filed: Nov. 2, 2006
(86) PCT No.: PCT/US06/42906

§ 371 Date: Jul. 31, 2008

Related U.S. Application Data
(60) Provisional application No. 60/732,531, filed on Nov. 2, 2005.
(60) Provisional application No. 60/735,686, filed on Nov. 10, 2005.

(51) Int. Cl. C12Q 1/68

(52) U.S. Cl. 435/6

(57) ABSTRACT

Provided are quadruplex nucleotide sequences and methods for identifying interacting molecules.

### FIELD OF THE INVENTION

The invention relates to quadruplex nucleotide sequences and methods for identifying interacting molecules.

### BACKGROUND

Developments in molecular biology have led to an understanding of how certain therapeutic compounds interact with molecular targets and lead to a modified physiological condition. Specificity of therapeutic compounds for their targets is derived in part from interactions between complementary structural elements in the target molecule and the therapeutic compound. A greater variety of target structural elements in the target leads to the possibility of unique and specific target/compound interactions. Because polypeptides are structurally diverse, researchers have focused on this class of targets for the design of specific therapeutic molecules.

In addition to therapeutic compounds that target polypeptides, researchers also have identified compounds that target DNA. Some of these compounds are effective anticancer agents and have led to significant increases in the survival of cancer patients. Unfortunately, however, these DNA targeting compounds do not act specifically on cancer cells and therefore are extremely toxic. Their unspecific action may be due to the fact that DNA often requires the uniformity of Watson-Crick duplex structures for compactly storing information within the human genome. This uniformity of DNA structure does not offer a structurally diverse population of DNA molecules that can be specifically targeted.

Nevertheless, there are some exceptions to this structural uniformity, as certain DNA sequences can form unique secondary structures. For example, intermittent runs of guanines can form G quadruplex structures, and complementary runs of cytosines can form i motif structures. Formation of G quadruplex and i-motif structures occurs when a particular region of duplex DNA transitions from Watson-Crick base pairing to intermolecular and intramolecular single-stranded structures.

### SUMMARY

Provided are isolated nucleic acids containing a nucleotide sequence that can comprise (a) a C-rich or G-rich sequence from human genomic DNA, (b) a complement of (a), (c) an encoded RNA nucleotide sequence of (a), (d) an encoded RNA nucleotide sequence of (b), or substantially identical variant nucleotide sequence of the foregoing. In certain embodiments, the nucleotide sequence comprises (a) one or more nucleotide sequences of Table A, (b) a complementary nucleotide sequence of (a), (c) an RNA nucleotide sequence encoded by (a), (d) an RNA nucleotide sequence encoded by (b), or (e) a substantially identical variant nucleotide sequence of the foregoing. The nucleotide sequence may conform to the motif ((G₃₊)N₁₋₇)₃G₃₊ or ((C₃₊)N₁₋₇)₃C₃₊, where “3+” is three or more nucleotides, C is cytosine, G is guanine and N is any nucleotide. The nucleotide sequence in some embodiments is in or near a region of DNA transcribed into RNA in a polymerase II-directed process. In polymerase II-directed processes, DNA generally is transcribed into a nascent RNA (“pre-RNA”), and the nascent RNA is processed into messenger RNA (“mRNA”). In some embodiments, the nucleotide sequence is in or near a region of DNA that is replicated (e.g., telomere DNA). A nucleotide sequence often is capable of adopting a quadruplex structure (described in greater detail hereafter). In certain embodiments, the nucleotide sequence is a G-rich or C-rich sequence in or near one of the following genes or regions: c-myc, MAX, c-myb, Vav, HIF-1a, Hmga2, PDGFA, PDGFB/c-sis, Her2-neu, EGFr, VEGF, TGF-B3, c-abl, c-src, RET, Bcl-2, MCL-1, Cyclin D1/Bcl-1, Cyclin A1, Ha-ras, DHFR & MRP 1, SPARC, Telomere, Insulin promoter, Cystatin B Promoter, FMR1 promoter, K-Ras, c-Kit and MAZ.

Also provided herein is a method for identifying a molecule that binds to a nucleic acid, which comprises contacting a nucleic acid described above; and detecting the amount of the compound bound or not bound to the nucleic acid, whereby the test molecule is identified as a molecule that binds to the nucleic acid containing the human nucleotide sequence when less of the compound binds to the nucleic acid in the presence of the test molecule than in the absence of the test molecule. The compound sometimes is in association with a detectable label, and is radiolabled in some embodiments. The compound is a quinolone or a porphyrin in certain embodiments. The nucleic acid sometimes is in association with a solid phase in certain embodiments. The test molecule is a quinolone derivative in certain embodiments, and the quinolone derivative sometimes is a compound of Tables 1A-1C, Table 2, Table 3 or Table 4. The nucleotide sequence sometimes is a DNA nucleotide sequence, at times is a RNA nucleotide sequence, and sometimes the nucleic acid comprises one or more nucleotide analogs or derivatives.

Provided also is a method for identifying a molecule that causes displacement of a protein from a nucleic acid, which comprises contacting a nucleic acid described above and a protein with a test molecule; and detecting the amount of the nucleic acid bound or not bound to the protein, whereby the test molecule is identified as a molecule that causes protein displacement when less of the nucleic acid binds to the protein in the presence of the test molecule than in the absence of the test molecule. In certain embodiments the protein is in association with a detectable label or is in association with a solid phase. The nucleic acid sometimes is in association with a detectable label or sometimes is in association with a solid phase in certain embodiments. The test molecule is a quinolone derivative in certain embodiments, and the quinolone derivative sometimes is a compound of Tables 1A-1C, Table 2, Table 3 or Table 4. The nucleotide sequence sometimes is a DNA nucleotide sequence, at times is a RNA nucleotide sequence, and sometimes the nucleic acid comprises one or more nucleotide analogs or derivatives.

Also provided is a method of identifying a modulator of nucleic acid synthesis, which comprises contacting a template nucleic acid, a primer oligonucleotide having a nucleotide sequence complementary to a template nucleic acid nucleotide sequence, extension nucleotides, a polymerase and a test molecule under conditions that allow the primer oligonucleotide to hybridize to the template nucleic acid, wherein the template nucleic acid comprises a nucleotide sequence described above; and detecting the presence, absence or amount of an elongated primer product synthesized by extension of the primer nucleic acid, whereby the test molecule is identified as a modulator of nucleic acid synthesis when a different amount of an elongated primer product is synthesized in the presence of the test molecule than in the absence of the test molecule. The template nucleic acid sometimes is DNA and is RNA in certain embodiments. In some embodiments, the polymerase is a DNA polymerase, and sometimes is an RNA polymerase.

### DETAILED DESCRIPTION

Nucleic acids, compounds and related methods described herein are useful in a variety of applications. For example, the nucleotide sequences described herein can serve as targets for screening interacting molecules (e.g., in screening assays). The interacting molecules may be utilized as novel therapeutics or for the discovery of novel therapeutics. Nucleic acid interacting molecules can serve as tools for identifying other target nucleotide sequences (e.g., target screening assays) or other interacting molecules (e.g., competition screening assays). The nucleotide sequences or complementary sequences thereof also can be utilized as aptamers or serve as basis for generating aptamers. The aptamers can be utilized as therapeutics or in assays for identifying novel interacting molecules.

Nucleic Acids

Provided are isolated nucleic acids containing a nucleotide sequence that can comprise (a) a C-rich or G-rich sequence from human genomic DNA, (b) a complement of (a), (c) an encoded RNA nucleotide sequence of (a), (d) an encoded RNA nucleotide sequence of (b), or substantially identical variant thereof. In certain embodiments, the nucleotide sequence comprises (a) one or more nucleotide sequences of Table A, (b) a complementary nucleotide sequence of (a), (c) an RNA nucleotide sequence encoded by (a), (d) an RNA nucleotide sequence encoded by (b), or (e) a substantially identical variant nucleotide sequence of the foregoing. The nucleotide sequence may conform to the motif ((G₃₊)N₁₋₇)₃G₃₊ or ((C₃₊)N₁₋₇)₃C₃₊, where “3+” is three or more nucleotides, C is cytosine, G is guanine and N is any nucleotide. The nucleotide sequence in some embodiments is in or near a region of DNA transcribed into RNA in a polymerase II-directed process. In polymerase II-directed processes, DNA generally is transcribed into a nascent RNA (“pre-RNA”), and the nascent RNA is processed into messenger RNA (“mRNA”). In some embodiments, the nucleotide sequence is in or near a region of DNA that is replicated (e.g., telomere DNA). A nucleotide sequence often is capable of adopting a quadruplex structure (described in greater detail hereafter). In certain embodiments, the nucleotide sequence is a G-rich or C-rich sequence in or near one of the following genes or regions: c-myc, MAX, c-myb, Vav, HIF-1a, Hmga2, PDGFA, PDGFB/c-sis, Her2-neu, EGFr, VEGF, TGF-B3, c-abl, c-src, RET, Bcl-2, MCL-1, Cyclin D1/Bcl-1, Cyclin A1, Ha-ras, DHFR & MRP1, SPARC, Telomere, Insulin promoter, Cystatin B Promoter, FMR1 promoter, K-Ras, c-Kit and MAZ.

In Table A, certain sequences are repeated, and the number of repeats for certain sequences are designated by n or m. For certain sequences designated n times, n is three (3) or more, such as 3 to 500, 3 to 100, 3 to 10, 3 to 8, 3 to 7, 3 to 6, 3 to 5 or 3 to 4 times, for example. For certain sequences repeated m times, m is two (2) or more, such as 2 to 500, 2 to 100, 2 to 10, 2 to 8, 2 to 7, 2 to 6, 2 to 5, 2 to 4 or 2 to 3 times, for example.

TABLE A

| | |
|---|---|
| c-myc | TGGGGAGGGTGGGGAGGGTGGGGAAGG |
| c-myc | CCTTCCCCACCCTCCCCACCCTCCCCA |
| MAX | CGGCGGCGGGGAGGGGAAGGGGTGAAGGGGAGGGGGA |
| MAX | TCCCCCTCCCCTTCACCCCTTCCCCTCCCCGCCGCCG |
| c-myb | AGAGGAGGAGGAGGACACGGAGGAGGAGGAGAAGGAGGAGGAGGAA |
| c-myb | TTCCTCCTCCTCCTTCTCCTCCTCCTCCGTGTCCTCCTCCTCCTCT |
| Vav | TGGAGGAGGAGGAG |
| Vav | CTCCTCCTCCTCCA |
| HIF-1a | GCGCGGGGAGGGGAGAGGGGGCGGGAGCGCG |
| HIF-1a | CGCGCTCCCGCCCCCTCTCCCCTCCCCGCGC |
| Hmga2 | AGAGAAGAGGGGAGGAGGAGGAGGAGAGGAGGAGGCGC |
| Hmga2 | GCGCCTCCTCCTCTCCTCCTCCTCCTCCCCTCTTCTCT |
| PDGFA | GGGGGGGCGGGGGCGGGGGCGGGGGAGGGGC |
| PDGFA | GCCCCTCCCCCGCCCCCGCCCCCGCCCCCCC |
| PDGFB/c-sis | GGGGGGGGACGCGGGAGCTGGGGGAGGGCTTGGGGCCAGGGCGGG |
| | GCGCTTAGGGGG |
| PDGFB/c-sis | CCCCCTAAGCGCCCCGCCCTGGCCCCAAGCCCTCCCCCAGCTCCC |
| | GCGTCCCCCCCC |
| Her2-neu | AGGAGAAGGAGGAGGTGGAGGAGGAGG |
| Her2-neu | CCTCCTCCTCCACCTCCTCCTTCTCCT |
| EGFr | AGGAGGAGGAGAATGCGAGGAGGAGGGAGGAGA |
| EGFr | TCTCCTCCCTCCTCCTCGCATTCTCCTCCTCCT |
| VEGF | GGGGCGGGCCGGGGGCGGGGTCCCGGCGGGGCGGAG |
| VEGF | CTCCGCCCCGCCGGGACCCCGCCCCCGGCCCGCCCC |
| TGF-B3 | GGGGTGGGGGAGGGAGGGA |
| TGF-B3 | TCCCTCCCTCCCCCACCCC |
| c-ab1 | AGGAAGGGGAGGGCCGGGGGGAGGTGGC |
| c-ab1 | GCCACCTCCCCCCGGCCCTCCCCTTCCT |
| c-src | CGGGAGGAGGAGGAAGGAGGAAGCGCG |
| c-src | CGCGCTTCCTCCTTCCTCCTCCTCCCG |
| RET | AGGGGCGGGGCGGGGCGGGGGC |
| RET | GCCCCCGCCCCGCCCCGCCCCT |
| Bcl-2 | AGGGGCGGGCGCGGGAGGAAGGGGGCGGGAGCGGGGCTG |
| Bcl-2 | CAGCCCCGCTCCCGCCCCCTTCCTCCCGCGCCCGCCCCT |
| MCL-1 | CCGGCCGGGCCGGGGCGGGGCCGGGGCCGGGGCCGGGGC |
| MCL-1 | GCCCCGGCCCCGGCCCCGGCCCCGCCCCGGCCCGGCCGG |
| Cyclin D1/Bcl-1 | GGGGGGCGGGGGCGGGCGCAGGGGGAGGGGGC |
| Cyclin D1/Bcl-1 | GCCCCCTCCCCCTGCGCCCGCCCCCGCCCCCC |
| Cyclin A1 | TGGGGCGGGGCAGGGCGGGGCAGGGT |
| Cyclin A1 | ACCCTGCCCCGCCCTGCCCCGCCCCA |
| Ha-ras | CGGGGCGGGGCGGGGGCGGGGGC |
| Ha-ras | GCCCCCGCCCCCGCCCCGCCCCG |
| DHFR & MRP1 | CGGGGCGGGGGGGCGGGGC |
| DHFR & MRP1 | GCCCCGCCCCCCCGCCCCG |
| SPARC | GGAGGAGGAGGAGGAGGAGGAGGA |
| SPARC | TCCTCCTCCTCCTCCTCCTCCTCC |
| Telomere | (GGGTTA)n |
| Telomere | (TAACCC)n |
| Insulin promoter | (ACAGGGGTGTGGGG)m |
| Insulin promoter | (CCCCACACCCCTGT)m |
| Cystatin B Promoter | (CGCGGGGCGGGG)m |
| Cystatin B Promoter | (CCCCGCCCCGCG)m |
| FMR1 promoter | (GGC)n |
| FMR1 promoter | (GCC)n |
| K-Ras | GGGAGGGAGGGAAGGAGGGAGGGAGGGAG |
| K-Ras | CTCCCTCCCTCCCTCCTTCCCTCCCTCCC |
| c-Kit | AGGGAGGGCGCTGGGAGGAGGG |
| c-Kit | CCCTCCTCCCAGCGCCCTCCCT |
| MAZ | AGGGGGGTGGGGGCGGGGGGAGGGA |
| MAZ | TCCCTCCCCCCGCCCCCACCCCCCT |

A nucleic acid may be single-stranded, double-stranded, triplex, linear or circular. The nucleic acid sometimes is a DNA, at times is RNA, and may comprise one or more nucleotide derivatives or analogs of the foregoing (e.g., 1, 2, 3, 4, 5, 6, 7, 8, 9, 10 or more analog or derivative nucleotides). In some embodiments, the nucleic acid is entirely comprised of one or more analog or derivative nucleotides, and sometimes the nucleic acid is composed of about 50% or fewer, about 25% or fewer, about 10% or fewer or about 5% or fewer analog or derivative nucleotide bases. One or more nucleotides in an analog or derivative nucleic acid may comprise a nucleobase modification or backbone modification, such as a ribose or phosphate modification (e.g., ribosepeptide nucleic acid (PNA) or phosphothioate linkages), as compared to a RNA or DNA nucleotide. Nucleotide analogs and derivatives are known to the person of ordinary skill in the art, and non-limiting examples of such modifications are set forth in U.S. Pat. No. 6,455,308 (Freier et al.); in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; and in WIPO publications WO 00/56746 and WO 01/14398. Methods for synthesizing nucleic acids comprising such analogs or derivatives are disclosed, for example, in the patent publications cited above, and in U.S. Pat. Nos. 6,455,308; 5,614,622; 5,739,314; 5,955,599; 5,962,674; 6,117,992; and in WO 00/75372.

A nucleic acid or nucleotide sequence therein sometimes is about 8 to about 80 nucleotides in length, at times about 8 to about 50 nucleotides in length, and sometimes from about 10 to about 30 nucleotides in length. In some embodiments, the nucleic acid or nucleotide sequence therein sometimes is about 500 or fewer, about 400 or fewer, about 300 or fewer, about 200 or fewer, about 150 or fewer, about 100 or fewer, about 90 or fewer, about 80 or fewer, about 70 or fewer, about 60 or fewer, or about 50 or fewer nucleotides in length, and sometimes is about 40 or fewer, about 35 or fewer, about 30 or fewer, about 25 or fewer, about 20 or fewer, or about 15 or fewer nucleotides in length. A nucleic acid sometimes is larger than the foregoing lengths, such as in embodiments in which it is in plasmid form, and can be about 600, about 700, about 800, about 900, about 1000, about 1100, about 1200, about 1300, or about 1400 base pairs in length or longer in certain embodiments.

Nucleic acids described herein often are isolated. The term “isolated” as used herein refers to material removed from its original environment (e.g., the natural environment if it is naturally occurring, or a host cell if expressed exogenously), often is purified from other materials in an original environment, and thus is altered “by the hand of man” from its original environment. The term “purified” as used herein with reference to molecules does not refer to absolute purity. Rather, “purified” refers to a substance in a composition that contains fewer substance species in the same class (e.g., nucleic acid or protein species) other than the substance of interest in comparison to the sample from which it originated. The term “purified” refers to a substance in a composition that contains fewer nucleic acid species other than the nucleic acid of interest in comparison to the sample from which it originated. Sometimes, a nucleic acid is “substantially pure,” indicating that the nucleic acid represents at least 50% of nucleic acid on a mass basis of the composition. Often, a substantially pure nucleic acid is at least 75% pure on a mass basis of the composition, and sometimes at least 95% pure on a mass basis of the composition. The nucleic acid may be purified from a biological source and/or may be manufactured. Nucleic acid manufacture processes (e.g., chemical synthesis and recombinant DNA processes) and purification processes are known to the person of ordinary skill in the art. For example, synthetic oligonucleotides can be synthesized using standard methods and equipment, such as by using an ABI™3900 High Throughput DNA Synthesizer, which is available from Applied Biosystems (Foster City, Calif.).

As described above, a nucleic acid may comprise a substantially identical sequence variant of a nucleotide sequence described herein. The term “substantially identical variant” as used herein refers to a nucleotide sequence sharing sequence identity to a nucleotide sequence described. Included are nucleotide sequences 55% or more, 60% or more, 65% or more, 70% or more, 75% or more, 80% or more, 85% or more, 90% or more, 91% or more, 92% or more, 93% or more, 94% or more, 95% or more, 96% or more, 97% or more, 98% or more or 99% or more sequence identity to a nucleotide sequence described herein. In certain embodiments, the substantially identical variant is 91% or more identical to a nucleotide sequence described herein. One test for determining whether two nucleotide sequences are substantially identical is to determine the percent of identical nucleotide sequences shared.

Calculations of sequence identity can be performed as follows. Sequences are aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is sometimes 30% or more, 40% or more, 50% or more, often 60% or more, and more often 70% or more, 80% or more, 90% or more, or 100% of the length of the reference sequence. The nucleotides or amino acids at corresponding nucleotide or polypeptide positions, respectively, are then compared among the two sequences. When a position in the first sequence is occupied by the same nucleotide or amino acid as the corresponding position in the second sequence, the nucleotides or amino acids are deemed to be identical at that position. The percent identity between the two sequences is a function of the number of identical positions shared by the sequences, taking into account the number of gaps, and the length of each gap, introduced for optimal alignment of the two sequences. Comparison of sequences and determination of percent identity between two sequences can be accomplished using a mathematical algorithm. Percent identity between two amino acid or nucleotide sequences can be determined using the algorithm of Meyers & Miller, CABIOS 4: 11-17 (1989), which has been incorporated into the ALIGN program (version 2.0), using a PAM120 weight residue table, a gap length penalty of 12 and a gap penalty of 4. Also, percent identity between two amino acid sequences can be determined using the Needleman & Wunsch, J. Mol. Biol. 48: 444-453 (1970) algorithm which has been incorporated into the GAP program in the GCG software package (available at the http address www.gcg.com), using either a Blossum 62 matrix or a PAM250 matrix, and a gap weight of 16, 14, 12, 10, 8, 6, or 4 and a length weight of 1, 2, 3, 4, 5, or 6. Percent identity between two nucleotide sequences can be determined using the GAP program in the GCG software package (available at http address www.gcg.com), using a NWSgapdna.CMP matrix and a gap weight of 40, 50, 60, 70, or 80 and a length weight of 1, 2, 3, 4, 5, or 6. A set of parameters often used is a Blossum 62 scoring matrix with a gap open penalty of 12, a gap extend penalty of 4, and a frameshift gap penalty of 5.

Another manner for determining whether two nucleic acids are substantially identical is to assess whether a polynucleotide homologous to one nucleic acid will hybridize to the other nucleic acid under stringent conditions. As use herein, the term “stringent conditions” refers to conditions for hybridization and washing. Stringent conditions are known to those skilled in the art and can be found in Current Protocols in Molecular Biology, John Wiley & Sons, N.Y., 6.3.1-6.3.6 (1989). Aqueous and non-aqueous methods are described in that reference and either can be used. An example of stringent hybridization conditions is hybridization in 6× sodium chloride/sodium citrate (SSC) at about 45° C., followed by one or more washes in 0.2×SSC, 0.1% SDS at 50° C. Another example of stringent hybridization conditions are hybridization in 6× sodium chloride/sodium citrate (SSC) at about 45° C., followed by one or more washes in 0.2×SSC, 0.1% SDS at 55° C. A further example of stringent hybridization conditions is hybridization in 6× sodium chloride/sodium citrate (SSC) at about 45° C., followed by one or more washes in 0.2×SSC, 0.1% SDS at 60° C. Often, stringent hybridization conditions are hybridization in 6× sodium chloride/sodium citrate (SSC) at about 45° C., followed by one or more washes in 0.2×SSC, 0.1% SDS at 65° C. More often, stringency conditions are 0.5M sodium phosphate, 7% SDS at 65° C., followed by one or more washes at 0.2×SSC, 1% SDS at 65° C.

Specific nucleotide sequences described herein can be used as “query sequences” to perform a search against public databases to identify other family members or related sequences, for example. The query sequences can be utilized to search for substantially identical sequences in organisms other than humans (e.g., apes, rodents (e.g., mice, rats, rabbits, guinea pigs), ungulates (e.g., equines, bovines, caprines, porcines), reptiles, amphibians and avians). Such searches can be performed using the NBLAST and XBLAST programs (version 2.0) of Altschul et al., J. Mol. Biol. 215: 403-10 (1990). BLAST nucleotide searches can be performed with the NBLAST program, score=100, wordlength=12 to obtain nucleotide sequences homologous to nucleotide sequences described herein. BLAST polypeptide searches can be performed with the XBLAST program, score=50, wordlength=3 to obtain amino acid sequences homologous to those encoded by SEQ ID NO: 1, 2, 3, 6, 7, 8, 13, 15, 17, 19, 21, 22, 23, 26 or 28. To obtain gapped alignments for comparison purposes, Gapped BLAST can be utilized as described in Altschul et al., Nucleic Acids Res. 25(17): 3389-3402 (1997). When utilizing BLAST and Gapped BLAST programs, default parameters of the respective programs (e.g., XBLAST and NBLAST) can be used (see the http address www.ncbi.nlm.nih.gov).

In some embodiments, an isolated nucleic acid can include a nucleotide sequence that encodes a nucleotide sequence described herein. In other embodiments, the nucleic acid includes a nucleotide sequence that encodes the complement of a nucleotide sequence described herein. For example, a nucleotide sequence described herein, or a sequence complementary to a nucleotide sequence described herein, may be included within a longer nucleotide sequence in the nucleic acid. The encoded nucleotide sequence sometimes is referred to herein as an “aptamer” and can be utilized in screening methods or as a therapeutic. In certain embodiments, the aptamer is complementary to a nucleotide sequence herein and can hybridize to a target nucleotide sequence. The hybridized aptamer may form a duplex or triplex with the target complementary nucleotide sequence, for example. The aptamer can be synthesized by the encoding sequence in an in vitro or in vivo system. When synthesized in vitro, an aptamer sometimes contains analog or derivative nucleotides. When synthesized in vivo, the encoding sequence may integrate into genomic DNA in the system or replicate autonomously from the genome (e.g., within a plasmid nucleic acid). An aptamer sometimes is selected by a measure of binding or hybridization affinity to a particular protein or nucleic acid target. In certain embodiments the aptamer may bind to one or more protein molecules within a cell or in plasma and induce a therapeutic response or be used as a method to detect the presence of the protein(s).

The isolated nucleic acid by be provided under conditions that allow formation of a quadruplex structure, and sometimes stabilize the structure. The term “quadruplex structure,” as used herein refers to a structure within a nucleic acid that includes one or more guanine-tetrad (G-tetrad) structures or cytosine-tetrad structures (C-tetrad or “i-motif”). G-tetrads can form in quadruplex structures via Hoogsteen hydrogen bonds. A quadruplex structure may be intermolecular (i.e., formed between two, three, four or more separate nucleic acids) or intramolecular (i.e., formed within a single nucleic acid). In some embodiments, a quadruplex-forming nucleic acid is capable of forming a parallel quadruplex structure having four parallel strands (e.g., propeller structure), antiparallel quadruplex structure having two stands that are antiparallel to the two parallel strands (e.g., chair or basket quadruplex structure) or a partially parallel quadruplex structure having one strand that is antiparallel to three parallel strands (e.g., a chair-eller or basket-eller quadruplex structure). Such structures are described in U.S. Patent Application Publication Nos. 2004/0005601 and PCT Application PCT/US2004/037789, for example. One or more quadruplex structures may form within a nucleic acid, and may form at one or more regions in the nucleic acid. Depending upon the length of the nucleic acid, the entire nucleic acid may form the quadruplex structure, and often a portion of the nucleic forms a particular quadruplex structure.

Conditions that allow quadruplex formation and stabilization are known to the person of ordinary skill in the art, and optimal quadruplex-forming conditions can be tested. Ion type, ion concentration, counteranion type and incubation time can be varied, and the artisan of ordinary skill can routinely determine whether a quadruplex conformation forms and is stabilized for a given set of conditions by utilizing the methods described herein. For example, cations (e.g., monovalent cations such as potassium) can stabilize quadruplex structures. The nucleic acid may be contacted in a solution containing ions for a particular time period, such as about 5 minutes, about 10 minutes, about 20 minutes, about 30 minutes, about 40 minutes, about 50 minutes or about 60 minutes or more, for example. A quadruplex structure is stabilized if it can form a functional quadruplex in solution, or if it can be detected in solution.

One nucleic acid sequence can give rise to different quadruplex orientations, where the different conformations depend in part upon the nucleotide sequence of the nucleic acid and conditions under which they form, such as the concentration of potassium ions present in the system and the time within which the quadruplex is allowed to form. Multiple conformations can be in equilibrium with one another, and can be in equilibrium with duplex nucleic acid if a complementary strand exists in the system. The equilibrium may be shifted to favor one conformation over another such that the favored conformation is present in a higher concentration or fraction over the other conformation or other conformations. The term “favor” or “stabilize” as used herein refers to one conformation being at a higher concentration or fraction relative to other conformations. The term “hinder” or “destabilize” as used herein refers to one conformation being at a lower concentration. One conformation may be favored over another conformation if it is present in the system at a fraction of 50% or greater, 75% or greater, or 80% or greater or 90% or greater with respect to another conformation (e.g., another quadruplex conformation, another paranemic conformation, or a duplex conformation). Conversely, one conformation may be hindered if it is present in the system at a fraction of 50% or less, 25% or less, or 20% or less and 10% or less, with respect to another conformation.

Equilibrium may be shifted to favor one quadruplex form over another form by methods described herein. A quadruplex forming region in a nucleic acid may be altered in a variety of manners. Alternations may result from an insertion, deletion, or substitution of one or more nucleotides. Substitutions can include a single nucleotide replacement of a nucleotide, such as a guanine that participates in a G-tetrad, where one, two, three, or four of more of such guanines in the quadruplex nucleic acid may be substituted. Also, one or more nucleotides near a guanine that participates in a G-tetrad may be deleted or substituted or one or more nucleotides may be inserted (e.g., within one, two, three or four nucleotides of a guanine that participates in a G-tetrad. A nucleotide may be substituted with a nucleotide analog or with another DNA or RNA nucleotide (e.g., replacement of a guanine with adenine, cytosine or thymine), for example. Ion concentrations and the time with which quadruplex DNA is contacted with certain ions can favor one conformation over another. Ion type, counterion type, ion concentration and incubation times can be varied to select for a particular quadruplex conformation. In addition, compounds that interact with quadruplex DNA may favor one form over the other and thereby stabilize a particular form.

Standard procedures for determining whether a quadruplex structure forms in a nucleic acid are known to the person of ordinary skill in the art. Also, different quadruplex conformations can be identified separately from one another using standard known procedures known to the person of ordinary skill in the art. Examples of such methods, such as characterizing quadruplex formation by polymerase arrest and circular dichroism, for example, are described in the Examples section hereafter.

Identification of Nucleotide Sequence Interacting Molecules

Provided are methods for identifying agents that interact with a nucleic acid described herein. Assay components, such as one or more nucleic acids and one or more test molecules, are contacted and the presence or absence of an interaction is observed. Assay components may be contacted in any convenient format and system by the artisan. As used herein, the term “system” refers to an environment that receives the assay components, including but not limited to microtiter plates (e.g., 96-well or 384-well plates), silicon chips having molecules immobilized thereon and optionally oriented in an array (see, e.g., U.S. Pat. No. 6,261,776 and Fodor, Nature 364: 555-556 (1993)), microfluidic devices (see, e.g., U.S. Pat. Nos. 6,440,722; 6,429,025; 6,379,974; and 6,316,781) and cell culture vessels. The system can include attendant equipment, such as signal detectors, robotic platforms, pipette dispensers and microscopes. A system sometimes is cell free, sometimes includes one or more cells, sometimes includes or is a cell sample from an animal (e.g., a biopsy, organ, appendage), and sometimes is a non-human animal. Cells may be extracted from any appropriate subject, such as a mouse, rat, hamster, rabbit, guinea pig, ungulate (e.g., equine, bovine, porcine), monkey, ape or human subject, for example.

The artisan can select test molecules and test conditions based upon the system utilized and the interaction and/or biological activity parameters monitored. Any type of test molecule can be utilized, including any reagent described herein, and can be selected from chemical compounds, antibodies and antibody fragments, binding partners and fragments, and nucleic acid molecules, for example. Specific embodiments of each class of such molecules are described hereafter. One or more test molecules may be added to a system in assays for identifying nucleic acid interacting molecules. Test molecules and other components can be added to the system in any suitable order. A sample exposed to a particular condition or test molecule often is compared to a sample not exposed to the condition or test molecule so that any changes in interactions or biological activities can be observed and/or quantified.

Assay systems sometimes are heterogeneous or homogeneous. In heterogeneous assays, one or more reagents and/or assay components are immobilized on a solid phase, and complexes are detected on the solid phase at the end of the reaction. In homogeneous assays, the entire reaction is carried out in a liquid phase. In either approach, the order of addition of reactants can be varied to obtain different information about the molecules being tested. For example, test compounds that agonize target molecule/binding partner interactions can be identified by conducting the reaction in the presence of the test molecule in a competition format. Alternatively, test molecules that agonize preformed complexes, e.g., molecules with higher binding constants that displace one of the components from the complex, can be tested by adding a test compound to the reaction mixture after complexes have been formed.

In a heterogeneous assay embodiment, one or more assay components are anchored to a solid surface (e.g., a microtiter plate), and a non-anchored component often is labeled, directly or indirectly. One or more assay components may be immobilized to a solid support in heterogeneous assay embodiments. The attachment between a component and the solid support may be covalent or non-covalent (see, e.g., U.S. Pat. No. 6,022,688 for non-covalent attachments). The term “solid support” or “solid phase” as used herein refers to a wide variety of materials including solids, semi-solids, gels, films, membranes, meshes, felts, composites, particles, and the like. Suitable solid phases include those developed and/or used as solid phases in solid phase binding assays (e.g., U.S. Pat. Nos. 6,261,776; 5,900,481; 6,133,436; and 6,022,688; WIPO publication WO 01/18234; chapter 9 of Immunoassay, E. P. Diamandis and T. K. Christopoulos eds., Academic Press: New York, 1996; Leon et al., Bioorg. Med. Chem. Lett. 8: 2997 (1998); Kessler et al., Agnew. Chem. Int. Ed. 40: 165 (2001); Smith et al., J. Comb. Med. 1: 326 (1999); Orain et al., Tetrahedron Lett. 42: 515 (2001); Papanikos et al., J. Am. Chem. Soc. 123: 2176 (2001); Gottschling et al., Bioorg. And Medicinal Chem. Lett. 11: 2997 (2001)). Examples of suitable solid phases include membrane filters, cellulose-based papers, beads (including polymeric, latex and paramagnetic particles), glass (e.g., glass slide), polyvinylidene fluoride (PVDF), nylon, silicon wafers, microchips, microparticles, nanoparticles, chromatography supports, TentaGels, AgroGels, PEGA gels, SPOCC gels, multiple-well plates (e.g., microtiter plate), nanotubes and the like that can be used by those of skill in the art to sequester molecules. The solid phase can be non-porous or porous. Assay components may be oriented on a solid phase in an array. Thus provided are arrays comprising one or more, two or more, three or more, etc., of assay components described herein (e.g., nucleic acids) immobilized at discrete sites on a solid support in an ordered array. Such arrays sometimes are high-density arrays, such as arrays in which each spot comprises at least 100 molecules per square centimeter.

A partner of the immobilized species sometimes is exposed to the coated surface with or without a test molecule in certain heterogeneous assay embodiments. After the reaction is complete, unreacted components are removed (e.g., by washing) such that a significant portion of any complexes formed remain immobilized on the solid surface. Where the non-immobilized species is pre-labeled, the detection of label immobilized on the surface is indicative of complex formation. Where the non-immobilized species is not pre-labeled, an indirect label can be used to detect complexes anchored to the surface (e.g., by using a labeled antibody specific for the initially non-immobilized species). Depending upon the order of addition of reaction components, test compounds that inhibit complex formation or disrupt preformed complexes can be detected.

In certain embodiments, a protein or peptide test molecule or assay component is linked to a phage via a phage coat protein. Molecules capable of interacting with the protein or peptide linked to the phage are immobilized to a solid phase, and phages displaying proteins or peptides that interact with the immobilized components adhere to the solid support. Nucleic acids from the adhered phages then are isolated and sequenced to determine the sequence of the protein or peptide that interacted with the components immobilized on the solid phase. Methods for displaying a wide variety of peptides or proteins as fusions with bacteriophage coat proteins are well known (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991)). Methods are also available for linking the test polypeptide to the N-terminus or the C-terminus of the phage coat protein. The original phage display system was disclosed, for example, in U.S. Pat. Nos. 5,096,815 and 5,198,346. This system used the filamentous phage M13, which required that the cloned protein be generated in E. coli and required translocation of the cloned protein across the E. coli inner membrane. Lytic bacteriophage vectors, such as lambda, T4 and T7 are more practical since they are independent of E. coli secretion. T7 is commercially available and described in U.S. Pat. Nos. 5,223,409; 5,403,484; 5,571,698; and 5,766,905.

In heterogeneous assay embodiments, the reaction can be conducted in a liquid phase in the presence or absence of test molecule, where the reaction products are separated from unreacted components, and the complexes are detected (e.g., using an immobilized antibody specific for one of the binding components to anchor any complexes formed in solution, and a labeled antibody specific for the other partner to detect anchored complexes). Again, depending upon the order of addition of reactants to the liquid phase, test compounds that inhibit complex or that disrupt preformed complexes can be identified.

In some homogeneous assay embodiments, a preformed complex comprising a reagent and/or other component is prepared. One or more components in the complex (e.g., nucleic acid, nucleolin protein, or nucleic acid binding compound) is labeled. In some embodiments, a signal generated by a label is quenched upon complex formation (e.g., U.S. Pat. No. 4,109,496 that utilizes this approach for immunoassays). Addition of a test molecule that competes with and displaces one of the species from the preformed complex can result in the generation of a signal above background or reduction in a signal. In this way, test substances that disrupt nucleic acid/test molecule complexes can be identified.

In an embodiment for identifying test molecules that antagonize or agonize formation of a complex comprising a nucleic acid, a reaction mixture containing components of the complex is prepared under conditions and for a time sufficient to allow complex formation. The reaction mixture often is provided in the presence or absence of the test molecule. The test molecule can be included initially in the reaction mixture, or can be added at a time subsequent to the addition of the target molecule and its binding partner. Control reaction mixtures are incubated without the test molecule or with a placebo. Formation of any complex is detected. Decreased formation of a complex in the reaction mixture containing test molecule as compared to in a control reaction mixture indicates that the molecule antagonizes complex formation. Alternatively, increased formation of a complex in the reaction mixture containing test molecule as compared to in a control reaction mixture indicates that the molecule agonizes target molecule/binding partner complex formation. In certain embodiments, complex formation nucleic acid/interacting molecule can be compared to complex formation of a modified nucleic acid/interacting molecule (e.g., nucleotide replacement in the nucleic acid). Such a comparison can be useful in cases where it is desirable to identify test molecules that modulate interactions of modified nucleic acid but not non-modified nucleic acid.

In some embodiments, the artisan detects the presence or absence of an interaction between assay components (e.g., a nucleic acid and a test molecule). As used herein, the term “interaction” typically refers to reversible binding of particular system components to one another, and such interactions can be quantified. A molecule may “specifically bind” to a target when it binds to the target with a degree of specificity compared to other molecules in the system (e.g., about 75% to about 95% or more of the molecule is bound to the target in the system). Often, binding affinity is quantified by plotting signal intensity as a function of a range of concentrations or amounts of a reagent, reactant or other system component. Quantified interactions can be expressed in terms of a concentration or amount of a reagent required for emission of a signal that is 50% of the maximum signal (IC₅₀). Also, quantified interactions can be expressed as a dissociation constant (K_{d} or Kᵢ) using kinetic methods known in the art. Kinetic parameters descriptive of interaction characteristics in the system can be assessed, including for example, assessing Kₘ, k_{cat}, kₒₙ, and/or k_{off} parameters.

A variety of signals can be detected to identify the presence, absence or amount of an interaction. One or more signals detected sometimes are emitted from one or more detectable labels linked to one or more assay components. In some embodiments, one or more assay components are linked to a detectable label. A detectable label can be covalently linked to an assay component, or may be in association with a component in a non-covalent linkage. Non-covalent linkages can be effected by a binding pair, where one binding pair member is in association with the assay component and the other binding pair member is in association with the detectable label. Any suitable binding pair can be utilized to effect a non-covalent linkage, including, but not limited to, antibody/antigen, antibody/antibody, antibody/antibody fragment, antibody/antibody receptor, antibody/protein A or protein G, hapten/anti-hapten, biotin/avidin, biotin/streptavidin, folic acid/folate binding protein, vitamin B12/intrinsic factor, nucleic acid/complementary nucleic acid (e.g., DNA, RNA, PNA). Covalent linkages also can be effected by a binding pair, such as a chemical reactive group/complementary chemical reactive group (e.g., sulfhydryl/maleimide, sulfhydryl/haloacetyl derivative, amine/isotriocyanate, amine/succinimidyl ester, and amine/sulfonyl halides). Methods for attaching such binding pairs to reagents and effecting binding are known to the artisan.

Any detectable label suitable for detection of an interaction can be appropriately selected and utilized by the artisan. Examples of detectable labels are fluorescent labels such as fluorescein, rhodamine, and others (e.g., Anantha, et al., Biochemistry (1998) 37:2709 2714; and Qu & Chaires, Methods Enzymol. (2000) 321:353 369); radioactive isotopes (e.g., ¹²⁵I, ¹³¹I, ³⁵S, ³¹P, ³²P, ¹⁴C, ³H, ⁷Be, ²⁸Mg, ⁵⁷Co, ⁶⁵Zn, ⁶⁷Cu, ⁶⁸Ge, ⁸²Sr, ⁸³Rb, ⁹⁵Tc, ⁹⁶Tc, ¹⁰³Pd, ¹⁰⁹Cd, and ¹²⁷Xe); light scattering labels (e.g., U.S. Pat. No. 6,214,560, and commercially available from Genicon Sciences Corporation, CA); chemiluminescent labels and enzyme substrates (e.g., dioxetanes and acridinium esters), enzymic or protein labels (e.g., green fluorescence protein (GFP) or color variant thereof, luciferase, peroxidase); other chromogenic labels or dyes (e.g., cyanine), and labels described previously.

A fluorescence signal generally is monitored in assays by exciting a fluorophore at a specific excitation wavelength and then detecting fluorescence emitted by the fluorophore at a different emission wavelength. Many nucleic acid interacting fluorophores and their attendant excitation and emission wavelengths are known (e.g., those described above). Standard methods for detecting fluorescent signals also are known, such as by using a fluorescence detector. Background fluorescence may be reduced in the system with the addition of photon reducing agents (see, e.g., U.S. Pat. No. 6,221,612), which can enhance the signal to noise ratio.

Another signal that can be detected is a change in refractive index at a solid optical surface, where the change is caused by the binding or release of a refractive index enhancing molecule near or at the optical surface. These methods for determining refractive index changes of an optical surface are based upon surface plasmon resonance (SPR). SPR is observed as a dip in light intensity reflected at a specific angle from the interface between an optically transparent material (e.g., glass) and a thin metal film (e.g., silver or gold). SPR depends upon the refractive index of the medium (e.g., a sample solution) close to the metal surface. A change of refractive index at the metal surface, such as by the adsorption or binding of material near the surface, will cause a corresponding shift in the angle at which SPR occurs. SPR signals and uses thereof are further exemplified in U.S. Pat. Nos. 5,641,640; 5,955,729; 6,127,183; 6,143,574; and 6,207,381, and WIPO publication WO 90/05295 and apparatuses for measuring SPR signals are commercially available (Biacore, Inc., Piscataway, N.J.). In certain embodiments, an assay component can be linked via a linker to a chip having an optically transparent material and a thin metal film, and interactions between and/or with the reagents can be detected by changes in refractive index.

Other signals representative of structure may also be detected, such as NMR spectral shifts (see, e.g., Arthanari & Bolton, Anti-Cancer Drug Design 14: 317-326 (1999)), mass spectrometric signals and fluorescence resonance energy transfer (FRET) signals (e.g., Lakowicz et al., U.S. Pat. No. 5,631,169; Stavrianopoulos et al. U.S. Pat. No. 4,868,103). In FRET approaches, a fluorophore label on a first, “donor” molecule is selected such that its emitted fluorescent energy will be absorbed by a fluorescent label on a second, “acceptor” molecule, which in turn is able to fluoresce due to the absorbed energy. Alternately, the “donor” polypeptide molecule may simply utilize the natural fluorescent energy of tryptophan residues. Labels are chosen that emit different wavelengths of light, such that the “acceptor” molecule label may be differentiated from that of the “donor”. Since the efficiency of energy transfer between the labels is related to the distance separating the molecules, the spatial relationship between the molecules can be assessed. In a situation in which binding occurs between the molecules, the fluorescent emission of the “acceptor” molecule label in the assay should be maximal. A FRET binding event can be conveniently measured using standard fluorometric detection means well known (e.g., using a fluorimeter). Molecules useful for FRET are known (e.g., fluorescein and terbium). FRET can be utilized to detect interactions in vitro or in vivo.

Interaction assays sometimes are performed in a heterogeneous format in which interactions are detected by monitoring detectable label in association with or not in association with a target linked to a solid phase. An example of such a format is an immunoprecipitation assay. Multiple separation processes are available, such as gel electrophoresis, chromatography, sedimentation (e.g., gradient sedimentation) and flow cytometry processes, for example. Flow cytometry processes include, for example, flow microfluorimetry (FMF) and fluorescence activated cell sorting (FACS); U.S. Pat. Nos. 6,090,919 (Cormack, et al.); 6,461,813 (Lorens); and 6,455,263 (Payan)). In some embodiments, cells also may be washed of unassociated detectable label, and detectable label associated with cellular components may be visualized (e.g., by microscopy).

In particular screening embodiments, provided is a method for identifying a molecule that binds to a nucleic acid containing a human nucleotide sequence, which comprises contacting a nucleic acid and a compound that binds to the nucleic acid with a test molecule, wherein the nucleic acid comprises a nucleotide sequence containing (a) one or more nucleotide sequences of Table A, (b) a complement of (a), (c) an RNA nucleotide sequence encoded by (a), (d) an RNA nucleotide sequence encoded by (b), or (e) a substantially identical variant nucleotide sequence of the foregoing; and detecting the amount of the compound bound or not bound to the nucleic acid, whereby the test molecule is identified as a molecule that binds to the nucleic acid containing the human nucleotide sequence when less of the compound binds to the nucleic acid in the presence of the test molecule than in the absence of the test molecule. The compound sometimes is in association with a detectable label, and at times is radiolabled. In certain embodiments, the compound is a quinolone analog (e.g., a quinolone analog described herein in Tables 1A-1C, Table 2, Table 3 or Table 4). Methods for radiolabeling compounds are known (e.g., U.S. patent application 60/718,021, filed Sep. 16, 2005, entitled METHODS FOR PREPARING RADIOACTIVE QUINOLONE ANALOGS). In some embodiments, the compound is a porphyrin (e.g., TMPyP4 or an expanded porphyrin described in U.S. patent application publication no. 20040110820 (e.g., Se₂SAP)). In the latter embodiments, fluorescence of the porphyrin sometimes is detected as the signal. The nucleic acid and/or another assay component sometimes is in association with a solid phase in certain embodiments. The nucleic acid may be DNA, RNA or an analog thereof, and may comprise a nucleotide sequence described above in specific embodiments. The nucleic acid may form a quadruplex, such as an intramolecular quadruplex.

In some screening embodiments, provided is a method for identifying a molecule that causes displacement of a protein from a nucleic acid, which comprises contacting a nucleic acid containing a nucleotide sequence and a protein with a test molecule, wherein the nucleic acid is capable of binding to the protein and the nucleotide sequence comprises (a) one or more nucleotide sequences of Table A, (b) a complement of (a), (c) an RNA nucleotide sequence encoded by (a), (d) an RNA nucleotide sequence encoded by (b), or (e) a substantially identical variant nucleotide sequence of the foregoing; and detecting the amount of the nucleic acid bound or not bound to the protein, whereby the test molecule is identified as a molecule that causes protein displacement when less of the nucleic acid binds to the protein in the presence of the test molecule than in the absence of the test molecule. In some embodiments, the protein is in association with a detectable label, and/or the protein may be in association with a solid phase. The nucleic acid sometimes is in association with a detectable label, and/or the nucleic acid may be in association with a solid phase in certain embodiments. Any convenient combination of the foregoing may be utilized. The nucleic acid may be DNA, RNA or an analog thereof, and may comprise a nucleotide sequence described above in specific embodiments. The nucleic acid may comprise G-quadruplex sequences and/or hairpin structures, sometimes composed of a five base pair stem and seven to ten nucleotide loop (e.g., U/GCCCGA motif). A protein that interacts with a quadruplex sequence may be utilized. Examples of such proteins include nucleolin, nucleolin binding protein and NM23 protein, for example. Any nucleolin protein may be utilized, such as a nucleolin having a sequence of accession no. NM_{—}005381, or a fragment or substantially identical sequence variant of the foregoing capable of binding a nucleic acid. Examples of nucleolin domains are RRM domains (e.g., amino acids 278-640) and RGG domains (e.g., amino acids 640-709). Any suitable nucleolin binding protein may be utilized, such as c-Myc (sequences under database accession nos. NM_{—}012603, AY679730, NP_{—}036735) or a fragment thereof (e.g., leucine zipper domain (positions 422-453), amino terminal domain positions 15-359; or helix-turn-helix domain (positions 366-425)); peroxisome proliferative activated receptor gamma coactivator protein (sequences under database accession nos. NM_{—}013261, AF106698, NP_{—}037393); Pr55 (Gag) of Human immunodeficiency virus 1 (sequences under accession no. NP_{—}057850); splicing factor, arginine/serine-rich 12 (sequences under accession nos. NM_{—}139168, AF459094, NP_{—}631907); and NM23-H2 (sequences under accession nos. NP_{—}036735.1, NW_{—}047336.1). In some embodiments the test molecule is a quinolone analog (e.g., a quinolone analog described herein in Tables 1A-1C, Table 2, Table 3 or Table 4).

In some screening embodiments, provided is a method of identifying a modulator of nucleic acid synthesis, which comprises contacting a template nucleic acid, a primer oligonucleotide having a nucleotide sequence complementary to a template nucleic acid nucleotide sequence, extension nucleotides, a polymerase and a test molecule under conditions that allow the primer oligonucleotide to hybridize to the template nucleic acid, wherein the template nucleic acid comprises (a) one or more nucleotide sequences of Table A, (b) a complement of (a), (c) an RNA nucleotide sequence encoded by (a), (d) an RNA nucleotide sequence encoded by (b), or (e) a substantially identical variant nucleotide sequence of the foregoing; and detecting the presence, absence or amount of an elongated primer product synthesized by extension of the primer nucleic acid, whereby the test molecule is identified as a modulator of nucleic acid synthesis when a different amount of an elongated primer product is synthesized in the presence of the test molecule than in the absence of the test molecule. In certain embodiments, the template nucleic acid is DNA and in other embodiments the template nucleic acid is RNA. The template nucleic acid sometimes comprises one or more nucleic acid analogs. In some embodiments, the polymerase is a DNA polymerase and in other embodiments, the polymerase is an RNA polymerase. Examples of suitable DNA and RNA polymerases are known and can be selected by the person of ordinary skill in the art. Examples of RNA polymerases include but are not limited to RNA polymerase II, SP6 RNA polymerase, T3 RNA polymerase, T7 RNA polymerase, RNA polymerase III and phage derived RNA polymerases. Examples of DNA polymerases include but are not limited to Pol I, II, II, IV or V, Taq polymerase and Klenow fragment.

Test molecules identified as having an effect in an assay described herein can be analyzed and compared to one another (e.g., ranked). Molecules identified as having an interaction or effect in a methods described herein are referred to as “candidate molecules.” Provided herein are candidate molecules identified by screening methods described herein, information descriptive of such candidate molecules, and methods of using candidate molecules (e.g., for therapeutic treatment of a condition).

Accordingly, provided is structural information descriptive of a candidate molecule identified by a method described herein. In certain embodiments, information descriptive of molecular structure (e.g., chemical formula or sequence information) sometimes is stored and/or renditioned as an image or as three-dimensional coordinates. The information often is stored and/or renditioned in computer readable form and sometimes is stored and organized in a database. In certain embodiments, the information may be transferred from one location to another using a physical medium (e.g., paper) or a computer readable medium (e.g., optical and/or magnetic storage or transmission medium, floppy disk, hard disk, random access memory, computer processing unit, facsimile signal, satellite signal, transmission over an internet or transmission over the world-wide web).

Nucleotide Sequence Interacting Molecules

Multiple types of nucleotide sequence interacting molecules can be constructed, identified and utilized by the person of ordinary skill in the art. Examples of such interacting molecules are compounds, nucleic acids and antibodies. Any of these types of molecules may be utilized as test molecules in assays described herein.

Compounds can be obtained using any of the numerous approaches in combinatorial library methods known in the art, including: biological libraries; peptoid libraries (libraries of molecules having the functionalities of peptides, but with a novel, non-peptide backbone which are resistant to enzymatic degradation but which nevertheless remain bioactive (see, e.g., Zuckermann et al., J. Med. Chem. 37: 2678-85 (1994)); spatially addressable parallel solid phase or solution phase libraries; synthetic library methods requiring deconvolution; “one-bead one-compound” library methods; and synthetic library methods using affinity chromatography selection. Biological library and peptoid library approaches are typically limited to peptide libraries, while the other approaches are applicable to peptide, non-peptide oligomer or small molecule libraries of compounds (Lam, Anticancer Drug Des. 12: 145, (1997)). Examples of methods for synthesizing molecular libraries are described, for example, in DeWitt et al., Proc. Natl. Acad. Sci. U.S.A. 90: 6909 (1993); Erb et al., Proc. Natl. Acad. Sci. USA 91: 11422 (1994); Zuckermann et al., J. Med. Chem. 37: 2678 (1994); Cho et al., Science 261: 1303 (1993); Carrell et al., Angew. Chem. Int. Ed. Engl. 33: 2059 (1994); Carell et al., Angew. Chem. Int. Ed. Engl. 33: 2061 (1994); and in Gallop et al., J. Med. Chem. 37: 1233 (1994). Libraries of compounds may be presented in solution (e.g., Houghten, Biotechniques 13: 412-421 (1992)), or on beads (Lam, Nature 354: 82-84 (1991)), chips (Fodor, Nature 364: 555-556 (1993)), bacteria or spores (Ladner, U.S. Pat. No. 5,223,409), plasmids (Cull et al., Proc. Natl. Acad. Sci. USA 89: 1865-1869 (1992)) or onphage (Scott and Smith, Science 249: 386-390 (1990); Devlin, Science 249: 404-406 (1990); Cwirla et al., Proc. Natl. Acad. Sci. 87: 6378-6382 (1990); Felici, J. Mol. Biol. 222: 301-310 (1991); Ladner supra.).

A compound sometimes is a small molecule. Small molecules include, but are not limited to, peptides, peptidomimetics (e.g., peptoids), amino acids, amino acid analogs, polynucleotides, polynucleotide analogs, nucleotides, nucleotide analogs, organic or inorganic compounds (i.e., including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

A nucleotide sequence interacting compound sometimes is a quinolone analog. In certain embodiments, the compound is of formula 1:

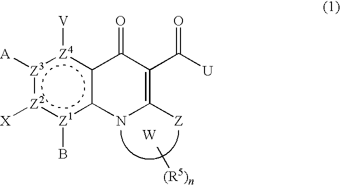

and pharmaceutically acceptable salts, esters and prodrugs thereof;

wherein B, X, A, or V is absent if Z¹, Z², Z³, or Z⁴, respectively, is N, and independently H, halo, azido, R², CH₂R², SR², OR² or NR¹R² if Z¹, Z², Z³, or Z⁴, respectively, is C; or

A and V, A and X, or X and B may form a carbocyclic ring, heterocyclic ring, aryl or heteroaryl, each of which may be optionally substituted and/or fused with a cyclic ring;

Z is O, S, NR¹, CH₂, or C═O;

Z¹, Z², Z³ and Z⁴ are C or N, provided any two N are non-adjacent;

W together with N and Z forms an optionally substituted 5- or 6-membered ring that is fused to an optionally substituted saturated or unsaturated ring; said saturated or unsaturated ring may contain a heteroatom and is monocyclic or fused with a single or multiple carbocyclic or heterocyclic rings;

U is R², OR², NR¹R², NR¹—(CR¹₂)ₙ—NR³R⁴, or N═CR¹R², wherein in N═CR¹R², R¹ and R² together with C may form a ring;

in each NR¹R², R¹ and R² together with N may form an optionally substituted ring;

in NR³R⁴, R³ and R⁴ together with N may form an optionally substituted ring;

R¹ and R³ are independently H or C₁₋₆ alkyl;

each R² is H, or a C₁₋₁₀ alkyl or C₂₋₁₀ alkenyl each optionally substituted with a halogen, one or more non-adjacent heteroatoms, a carbocyclic ring, a heterocyclic ring, an aryl or heteroaryl, wherein each ring is optionally substituted; or R² is an optionally substituted carbocyclic ring, heterocyclic ring, aryl or heteroaryl;

R⁴ is H, a C₁₋₁₀ alkyl or C₂₋₁₀ alkenyl optionally containing one or more non-adjacent heteroatoms selected from N, O and S, and optionally substituted with a carbocyclic or heterocyclic ring; or R³ and R⁴ together with N may form an optionally substituted ring;

each R⁵ is a substituent at any position on ring W; and is H, OR², amino, alkoxy, amido, halogen, cyano or an inorganic substituent; or R⁵ is C₁₋₆ alkyl, C₂₋₆ alkenyl, C₂₋₆ alkynyl, —CONHR¹, each optionally substituted by halo, carbonyl or one or more non-adjacent heteroatoms; or two adjacent R⁵ are linked to obtain a 5-6 membered optionally substituted carbocyclic or heterocyclic ring that may be fused to an additional optionally substituted carbocyclic or heterocyclic ring; and

n is 1-6.

In the above formula (1), B may be absent when Z¹ is N, or is H or a halogen when Z¹ is C. In certain embodiments, U sometimes is not H. In some embodiments, at least one of Z¹-Z⁴ is N when U is OH, OR² or NH₂.

In some embodiments, the compound has the general formula (2A) or (2B):

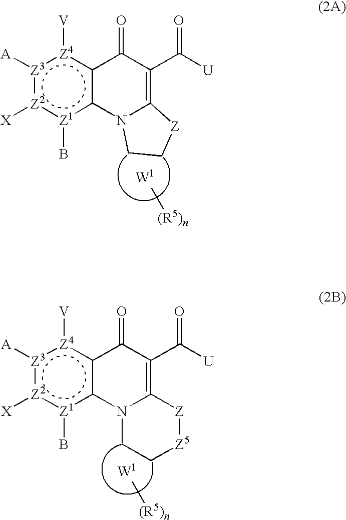

wherein A, B, V, X, U, Z, Z¹, Z², Z³, Z⁴, R⁵ and n are as defined in formula (1);

Z⁵ is O, NR¹, CR⁶, or C═O;

R⁶ is H, C₁₋₆ alkyl, hydroxyl, alkoxy, halo, amino or amido; and

Z and Z⁵ may optionally form a double bond.

In one embodiment, Z and Z⁵ in formula (2B) are non-adjacent atoms.

In some embodiments, compounds of the following formula (2C), or a pharmaceutically acceptable salt, ester or prodrug thereof, are utilized:

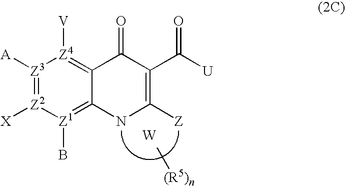

wherein substituents are set forth above.

In some embodiments, compounds of the following formula (2D), or a pharmaceutically acceptable salt, ester or prodrug thereof, are utilized:

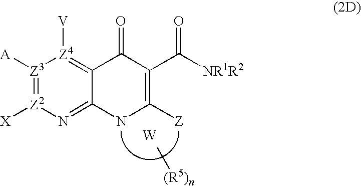

wherein substituents are set forth above. In certain embodiments, compounds of formula (2D) substantially arrest cell cycle, such as G1 phase arrest and/or S phase arrest, for example.

In certain aspects, the compound has the general formula (3):

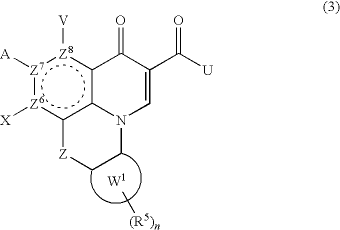

wherein A, U, V, X, R⁵, Z and n are as described above in formula (1);

W¹ is an optionally substituted aryl or heteroaryl, which may be monocyclic, or fused with a single or multiple ring and optionally containing a heteroatom; and

Z⁶, Z⁷, and Z⁸ are independently C or N, provided any two N are non-adjacent.

In the above formula (3), each of Z⁶, Z⁷, and Z⁸ may be C. In some embodiments, one or two of Z⁶, Z⁷, and Z⁸ is N, provided any two N are non-adjacent.

In the above formula, W together with N and Z in formula (1), (2C) or (2D), or W^{I} in formula (2A), (2B) or (3) forms an optionally substituted 5- or 6-membered ring that is fused to an optionally substituted aryl or heteroaryl selected from the group consisting of:

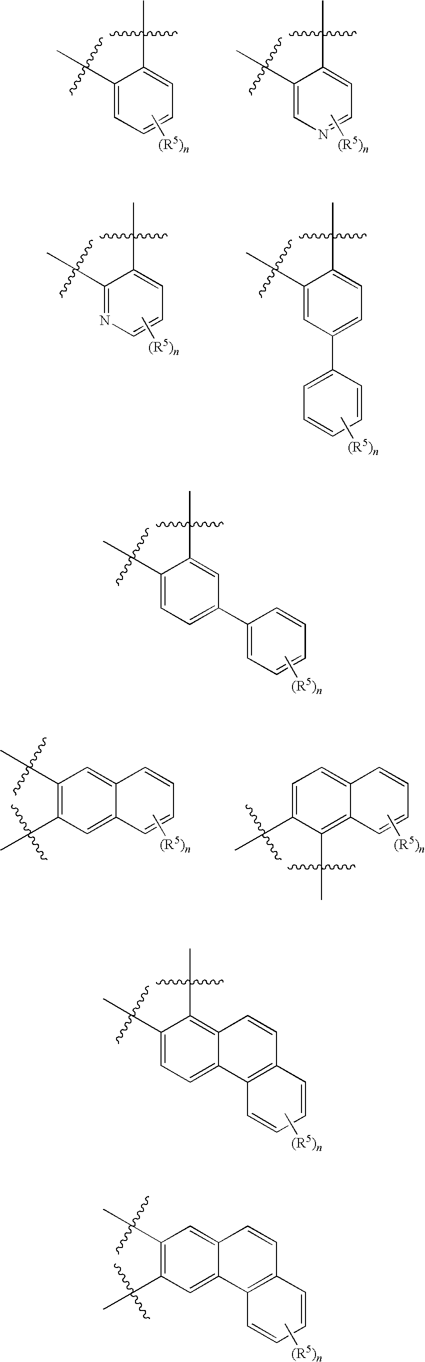

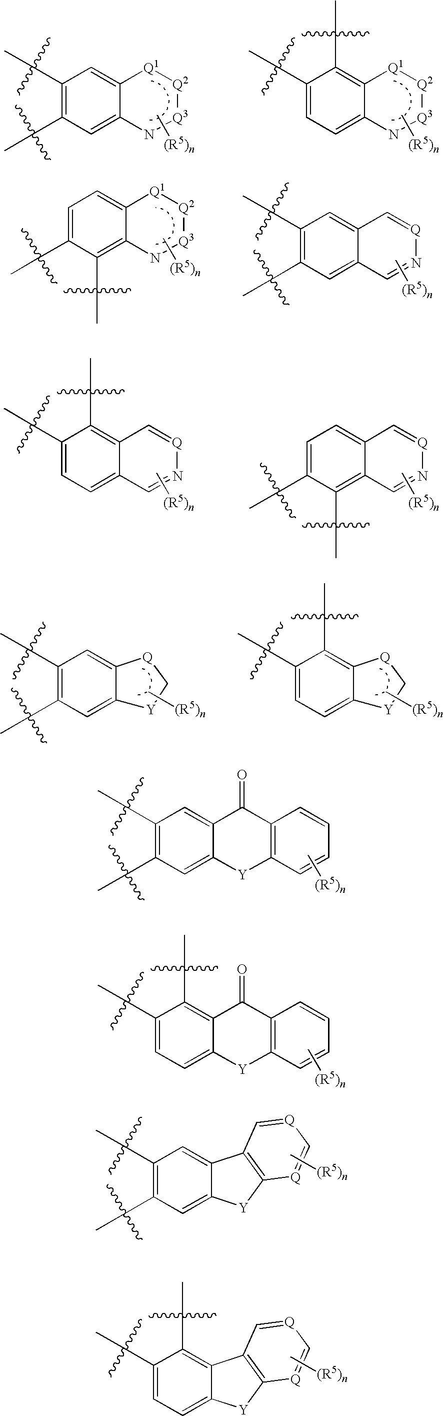

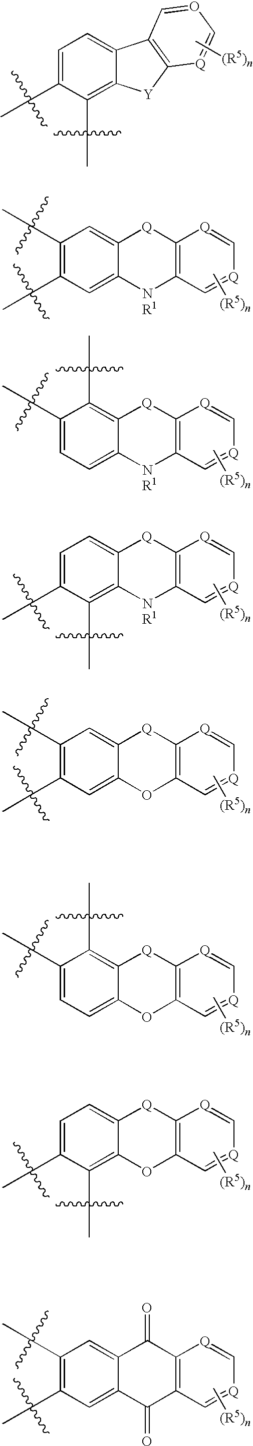

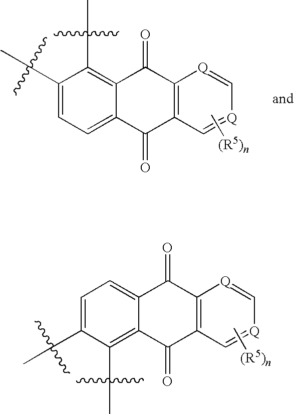

wherein each Q, Q¹, Q², and Q³ is independently CH or N;

Y is independently O, CH, C═O or NR¹;

n and R⁵ is as defined above.

In certain embodiments, W together with N and Z in formula (1), (2C) or (2D) form a group having the formula selected from the group consisting of

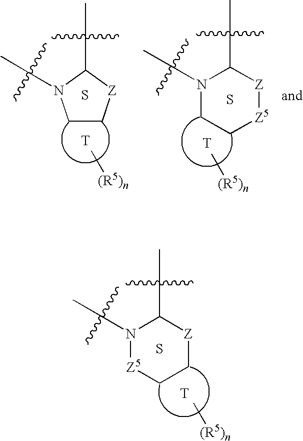

wherein Z is O, S, CR¹, NR¹, or C═O;

each Z⁵ is CR⁶, NR¹, or C═O, provided Z and Z⁵ if adjacent are not both NR¹;

each R¹ is H, C₁₋₆ alkyl, COR² or S(O)ₚR² wherein p is 1-2;

R⁶ is H, or a substituent known in the art, including but not limited to hydroxyl, alkyl, alkoxy, halo, amino, or amido; and

ring S and ring T may be saturated or unsaturated.

In some embodiments, W together with N and Z in formula (1), (2C) or (2D) forms a 5- or 6-membered ring that is fused to a phenyl. In other embodiments, W together with N and Z forms a 5- or 6-membered ring that is optionally fused to another ring, when U is NR¹R², provided U is not NH₂. In certain embodiments, W together with N and Z forms a 5- or 6-membered ring that is not fused to another ring, when U is NR¹R² (e.g., NH₂).

In the above formula (1), (2A), (2B), (2C) or (3), U may be NR¹R², wherein R¹ is H, and R² is a C₁₋₁₀ alkyl optionally substituted with a heteroatom, a C₃₋₆ cycloalkyl, aryl or a 5-14 membered heterocyclic ring containing one or more N, O or S. For example, R² may be a C₁₋₁₀ alkyl substituted with an optionally substituted morpholine, thiomorpholine, imidazole, aminodithiadazole, pyrrolidine, piperazine, pyridine or piperidine. In other examples, R¹ and R² together with N form an optionally substituted piperidine, pyrrolidine, piperazine, morpholine, thiomorpholine, imidazole, or aminodithiazole.

In some embodiments, U is NR¹—(CR¹₂)ₙ—NR³R⁴; n is 1-4; and R³ and R⁴ in NR³R⁴ together form an optionally substituted piperidine, pyrrolidine, piperazine, morpholine, thiomorpholine, imidazole, or aminodithiazole. In some examples, U is NH—(CH₂), —NR³R⁴ wherein R³ and R⁴ together with N form an optionally substituted pyrrolidine, which may be linked to (CH₂)ₙ at any position in the pyrrolidine ring. In one embodiment, R³ and R⁴ together with N form an N-methyl substituted pyrrolidine. In some embodiments, U is 2-(1-methylpyrrolidin-2-yl)ethylamino or (2-pyrrolidin-1-yl)ethanamino.

In the above formula (1), (2A-D) or (3), Z may be S or NR¹.

In some embodiments, at least one of B, X, or A in formula (1), (2A) or (2B) is halo and Z¹, Z², and Z³ are C. In other embodiments, X and A are not each H when Z² and Z³ are C. In the above formula (1), (2A) and (2B), V may be H. In particular embodiments, U is not OH.

In an embodiment, each of Z¹, Z², Z³ and Z⁴ in formula (1) or (2A-C) are C. In another embodiment, three of Z¹, Z², Z³ and Z⁴ is C, and the other is N. For example, Z¹, Z² and Z³ are C, and Z⁴ is N. Alternatively, Z¹, Z² and Z⁴ are C, and Z³ is N. In other examples, Z¹, Z³ and Z⁴ are C and Z² is N. In yet other examples, Z², Z³ and Z⁴ are C, and Z¹ is N.

In certain embodiments, two of Z¹, Z², Z³ and Z⁴ in formula (1) or (2A-C) are C, and the other two are non-adjacent nitrogens. For example, Z¹ and Z³ may be C, and Z² and Z⁴ are N. Alternatively, Z¹ and Z³ may be N, and Z² and Z⁴ may be C. In other examples, Z¹ and Z⁴ are N, and Z² and Z³ are C. In particular examples, W together with N and Z forms a 5- or 6-membered ring that is fused to a phenyl.

In some embodiments, each of B, X, A, and V in formula (1) or (2A-C) is H and Z¹-Z⁴ are C. In many embodiments, at least one of B, X, A, and V is H and the corresponding adjacent Z¹-Z⁴ atom is C. For example, any two of B, X, A, and V may be H. In one example, V and B may both be H. In other examples, any three of B, X, A, and V are H and the corresponding adjacent Z¹-Z⁴ atom is C.

In certain embodiments, one of B, X, A, and V is a halogen (e.g., fluorine) and the corresponding adjacent Z¹-Z⁴ is C. In other embodiments, two of X, A, and V are halogen or SR², wherein R² is a C₀₋₁₀ alkyl or C₂₋₁₀ alkenyl optionally substituted with a heteroatom, a carbocyclic ring, a heterocyclic ring, an aryl or a heteroaryl; and the corresponding adjacent Z²-Z⁴ is C. For example, each X and A may be a halogen. In other examples, each X and A if present may be SR², wherein R² is a C₀₋₁₀ alkyl substituted with phenyl or pyrazine. In yet other examples, V, A and X may be alkynyls, fluorinated alkyls such as CF₃, CH₂CF₃, perfluorinated alkyls, etc.; cyano, nitro, amides, sulfonyl amides, or carbonyl compounds such as COR².

In each of the above formulas, U, and X, V, and A if present may independently be NR¹R², wherein R¹ is H, and R² is a C₁₋₁₀ alkyl optionally substituted with a heteroatom, a C₃₋₆ cycloalkyl, aryl or a 5-14 membered heterocyclic ring containing one or more N, O or S. If more than one NR² moiety is present in a compound within the invention, as when both A and U are NR¹R² in a compound according to any one of the above formula, each R¹ and each R² is independently selected. In one example, R² is a C₁₋₁₀ alkyl substituted with an optionally substituted 5-14 membered heterocyclic ring. For example, R² may be a C₁₋₁₀ alkyl substituted with morpholine, thiomorpholine, imidazole, aminodithiadazole, pyrrolidine, piperazine, pyridine or piperidine. Alternatively, R¹ and R² together with N may form an optionally substituted heterocyclic ring containing one or more N, O or S. For example, R¹ and R² together with N may form piperidine, pyrrolidine, piperazine, morpholine, thiomorpholine, imidazole, or aminodithiazole.

Illustrative examples of optionally substituted heterocyclic rings include but are not limited to tetrahydrofuran, 1,3-dioxolane, 2,3-dihydrofuran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4-b]pyridine, piperazine, pyrazine, morpholine, thiomorpholine, imidazole, aminodithiadazole, imidazolidine-2,4-dione, benzimidazole, 1,3-dihydrobenzimidazol-2-one, indole, thiazole, benzothiazole, thiadiazole, thiophene, tetrahydro-thiophene 1,1-dioxide, diazepine, triazole, diazabicyclo[2.2.1]heptane, 2,5-diazabicyclo[2.2.1]heptane, and 2,3,4,4a,9,9a-hexahydro-1H-β-carboline.

In some embodiments, the compound has general formula (1), (2A-D) or (3), wherein:

each of A, V and B if present is independently H or halogen (e.g., chloro or fluoro);

X is —(R⁵)R¹R², wherein R⁵ is C or N and wherein in each ˜(R⁵)R¹R², R¹ and R² together may form an optionally substituted aryl or heteroaryl ring;

Z is NH or N-alkyl (e.g., N—CH₃);

W together with N and Z in formula (1), (2C) or (2D) or W¹ in formula (2A), (2B) or (3) forms an optionally substituted 5- or 6-membered ring that is fused with an optionally substituted aryl or heteroaryl ring; and

U is —R⁵R⁶—(CH₂)ₙ—CHR²—NR³R⁴, wherein R⁶ is H or C₁₋₁₀ alkyl and wherein in the —CHR²—NR³R⁴ moiety each R³ or R⁴ together with the C may form an optionally substituted heterocyclic or heteroaryl ring, or wherein in the —CHR²—NR³R⁴ moiety each R³ or R⁴ together with the N may form an optionally substituted carbocyclic, heterocyclic, aryl or heteroaryl ring.

In certain embodiments, the compound has formula (1), (2A-2D) or (3), wherein:

A if present is H or halogen (e.g., chloro or fluoro);

X if present is —(R⁵)R¹R², wherein R⁵ is C or N and wherein in each —(R⁵)R¹R², R¹ and R² together may form an optionally substituted aryl or heteroaryl ring;

Z is NH or N-alkyl (e.g., N—CH₃);

W together with N and Z in formula (1), (2C) or (2D) or W¹ in formula (2A), (2B) or (3) forms an optionally substituted 5- or 6-membered ring that is fused with an optionally substituted aryl or heteroaryl ring; and

U is —R⁵R⁶—(CH₂)ₙ—CHR²—NR³R⁴, wherein R⁶ is H or alkyl and wherein in the —CHR²—NR³R⁴ moiety each R³ or R⁴ together with the C may form an optionally substituted heterocyclic or heteroaryl ring, or wherein in the —CHR²—NR³R⁴ moiety each R³ or R⁴ together with the N may form an optionally substituted carbocyclic, heterocyclic, aryl or heteroaryl ring.

In each of the above formula, each optionally substituted moiety may be substituted with one or more halo, OR², NR¹R², carbamate, C₁₋₁₀ alkyl, C₂₋₁₀ alkenyl, each optionally substituted by halo, C═O, aryl or one or more heteroatoms; inorganic substituents, aryl, carbocyclic or a heterocyclic ring. Other substituents include but are not limited to alkynyl, cycloalkyl, fluorinated alkyls such as CF₃, CH₂CF₃, perfluorinated alkyls, etc.; oxygenated fluorinated alkyls such as OCF₃ or CH₂CF₃, etc.; cyano, nitro, COR², NR²COR², sulfonyl amides; NR²SOOR²; SR², SOR², COOR², CONR²₂, OCOR², OCOOR², OCONR²₂, NRCOOR², NRCONR²₂, NRC(NR)(NR²₂), NR(CO)NR²₂, and SOONR²₂, wherein each R² is as defined in formula 1.

As used herein, the term “alkyl” refers to a carbon-containing compound, and encompasses compounds containing one or more heteroatoms. The term “alkyl” also encompasses alkyls substituted with one or more substituents including but not limited to OR¹, amino, amido, halo, ═O, aryl, heterocyclic groups, or inorganic substituents.

As used herein, the term “carbocycle” refers to a cyclic compound containing only carbon atoms in the ring, whereas a “heterocycle” refers to a cyclic compound comprising a heteroatom. The carbocyclic and heterocyclic structures encompass compounds having monocyclic, bicyclic or multiple ring systems.

As used herein, the term “aryl” refers to a polyunsaturated, typically aromatic hydrocarbon substituent, whereas a “heteroaryl” or “heteroaromatic” refer to an aromatic ring containing a heteroatom. The aryl and heteroaryl structures encompass compounds having monocyclic, bicyclic or multiple ring systems.

As used herein, the term “heteroatom” refers to any atom that is not carbon or hydrogen, such as nitrogen, oxygen or sulfur.

Illustrative examples of heterocycles include but are not limited to tetrahydrofuran, 1,3-dioxolane, 2,3-dihydrofuran, pyran, tetrahydropyran, benzofuran, isobenzofuran, 1,3-dihydro-isobenzofuran, isoxazole, 4,5-dihydroisoxazole, piperidine, pyrrolidine, pyrrolidin-2-one, pyrrole, pyridine, pyrimidine, octahydro-pyrrolo[3,4-b]pyridine, piperazine, pyrazine, morpholine, thiomorpholine, imidazole, imidazolidine-2,4-dione, 1,3-dihydrobenzimidazol-2-one, indole, thiazole, benzothiazole, thiadiazole, thiophene, tetrahydro-thiophene 1,1-dioxide, diazepine, triazole, guanidine, diazabicyclo[2.2.1]heptane, 2,5-diazabicyclo[2.2.1]heptane, 2,3,4,4a,9,9a-hexahydro-1H-β-carboline, oxirane, oxetane, tetrahydropyran, dioxane, lactones, aziridine, azetidine, piperidine, lactams, and may also encompass heteroaryls. Other illustrative examples of heteroaryls include but are not limited to furan, pyrrole, pyridine, pyrimidine, imidazole, benzimidazole and triazole.

As used herein, the term “inorganic substituent” refers to substituents that do not contain carbon or contain carbon bound to elements other than hydrogen (e.g., elemental carbon, carbon monoxide, carbon dioxide, and carbonate). Examples of inorganic substituents include but are not limited to nitro, halogen, sulfonyls, sulfinyls, phosphates, etc.

Synthetic procedures for preparing the compounds of the present invention have been described in PCT/US05/011108 and PCT/US2005/26977, each of which is incorporated herein by reference in its entirety. Other variations in the synthetic procedures known to those with ordinary skill in the art may also be used to prepare the compounds of the present invention.

The compounds of the present invention may be chiral. As used herein, a chiral compound is a compound that is different from its mirror image, and has an enantiomer. Furthermore, the compounds may be racemic, or an isolated enantiomer or stereoisomer. Methods of synthesizing chiral compounds and resolving a racemic mixture of enantiomers are well known to those skilled in the art. See, e.g., March, “Advanced Organic Chemistry,” John Wiley and Sons, Inc., New York, (1985), which is incorporated herein by reference.

Illustrative examples of compounds having the above formula are shown in Table 1 (A-C), Tables 2-4, and in the Examples. The present invention also encompasses other compounds having any one formula (1), (2A-2D), (3) and (3A) comprising substituents U, A, X, V, and B independently selected from the substituents exemplified in the tables. For example, an isopropyl substituent in the compounds shown in Table 1A may be replaced with an acetyl substituent, or the N—CH₃ pyrrolidinyl in the fused ring may be replaced with a NH-pyrrolidinyl group. Furthermore, the fluoro group may be replaced with H. Thus, the present invention is not limited to the specific combination of substituents described in various embodiments below.

In some embodiments, compounds of the following formula (3A), or a pharmaceutically acceptable salt, ester or prodrug thereof, are utilized:

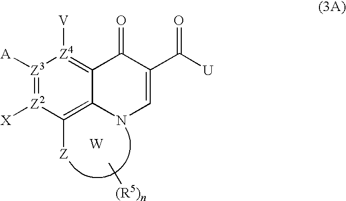

wherein substituents are set forth above.

In some embodiments, a compound has the following formula A-1,

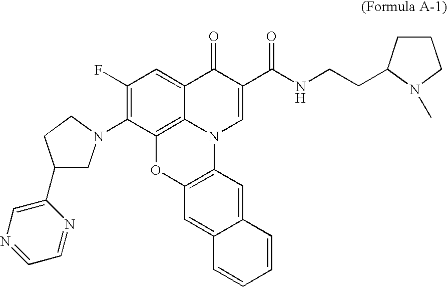

or a pharmaceutically acceptable salt, ester or prodrug thereof, and may be utilized in a method or composition described herein.

In some embodiments, a compound having the following formula B-1:

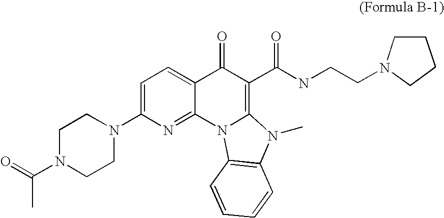

or a pharmaceutically acceptable salt, prodrug or ester thereof, may be utilized in a method or composition described herein.

In certain aspects, the compound is of formula 4, or a pharmaceutically acceptable salt, prodrug or ester thereof:

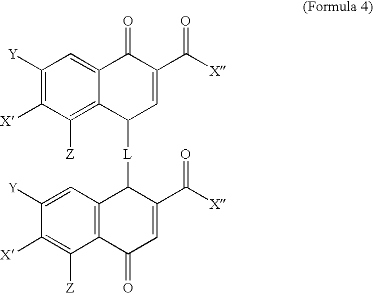

where X′ is hydroxy, alkoxy, carboxyl, halogen, CF₃, amino, amido, sulfide, 3-7 membered carbocycle or heterocycle, 5- or 6-membered aryl or heteroaryl, fused carbocycle or heterocycle, bicyclic compound, NR¹R², NCOR³, N(CH₂)ₙNR¹R², or N(CH₂)ₙR³, where the N in N(CH₂)ₙNR¹R² and N(CH₂)ₙR³ is optionally linked to a C1-10 alkyl, and each X′ is optionally linked to one or more substituents;

X″ is hydroxy, alkoxy, amino, amido, sulfide, 3-7 membered carbocycle or heterocycle, 5- or 6-membered aryl or heteroaryl, fused carbocycle or heterocycle, bicyclic compound, NR¹R², NCOR³, N(CH₂)ₙNR¹R², or N(CH₂)ₙR³, where the N in N(CH₂), NR¹R² and N(CH₂)ₙR³ is optionally linked to a C1-10 alkyl, and X″ is optionally linked to one or more substituents;

Y is H, halogen, or CF₃;

R¹, R² and R³ are independently H, C1-C6 alkyl, C1-C6 substituted alkyl, C3-C6 cycloalkyl, C1-C6 alkoxyl, carboxyl, imine, guanidine, 3-7 membered carbocycle or heterocycle, 5- or 6-membered aryl or heteroaryl, fused carbocycle or heterocycle, or bicyclic compound, where each R¹, R² and R³ are optionally linked to one or more substituents;

Z is a halogen;

and L is a linker having the formula Ar¹-L1-Ar², where Ar1 and Ar2 are aryl or heteroaryl.

In the above formula (4), L1 may be (CH₂)ₘ where m is 1-6, or a heteroatom optionally linked to another heteroatom such as a disulfide. Each of Ar1 and Ar2 may independently be aryl or heteroaryl, optionally substituted with one or more substituents. In one example, L is a [phenyl-S—S-phenyl] linker linking two quinolinone. In a particular embodiment, L is a [phenyl-S—S-phenyl] linker linking two identical quinoline species.

In the above formula (4), X″ may be hydroxy, alkoxy, amino, amido, sulfide, 3-7 membered carbocycle or heterocycle, 5- or 6-membered aryl or heteroaryl, fused carbocycle or heterocycle, bicyclic compound, NR¹R², NCOR³, N(CH₂)ₙ NR¹R², or N(CH₂)ₙR³, where the N in N(CH₂)ₙ NR¹R² and N(CH₂)ₙR³ is optionally linked to a C1-10 alkyl, and X″ is optionally linked to one or more substituents.

Illustrative examples of compounds of the foregoing formulae are set forth in Tables 1A-1C, Table 2, Table 3 and Table 4 attached hereto.

TABLE 1A

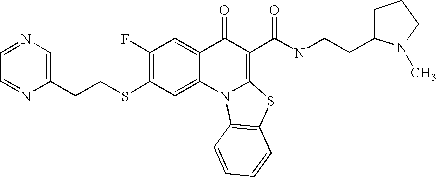

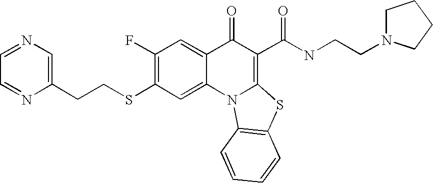

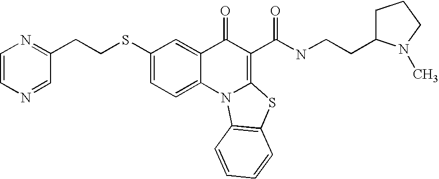

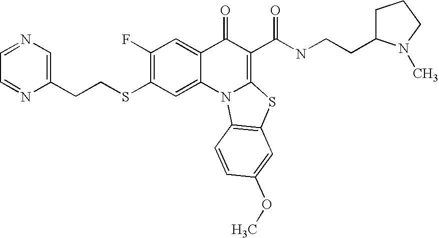

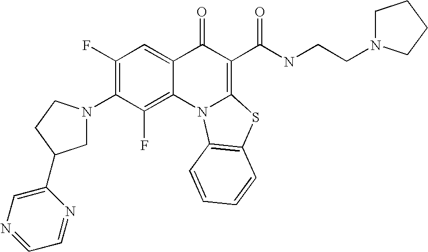

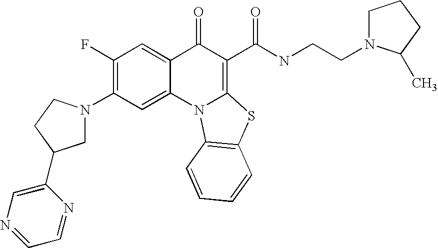

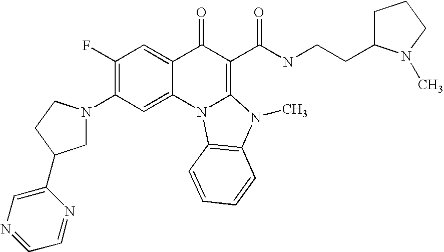

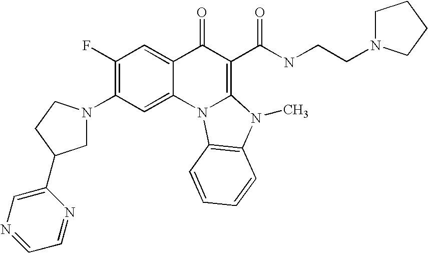

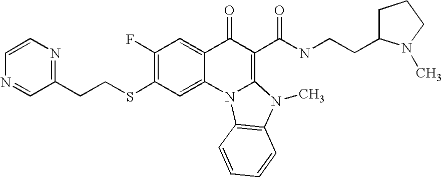

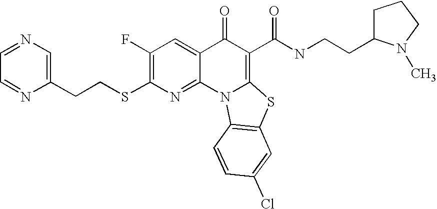

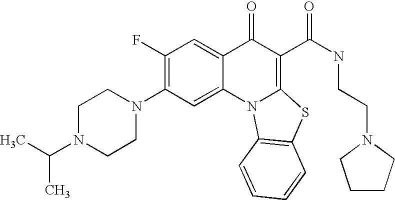

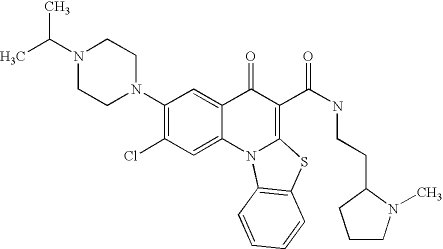

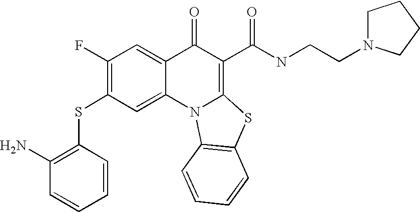

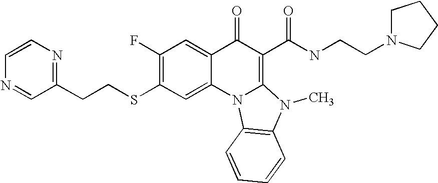

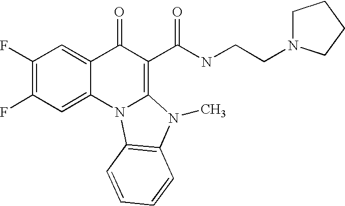

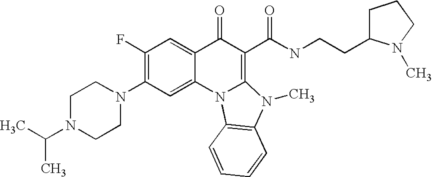

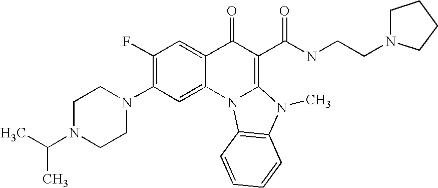

TABLE 1B

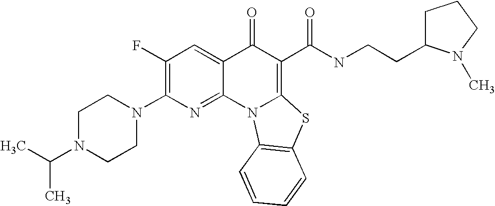

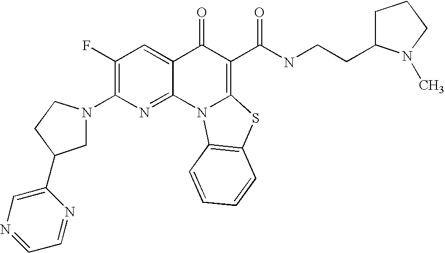

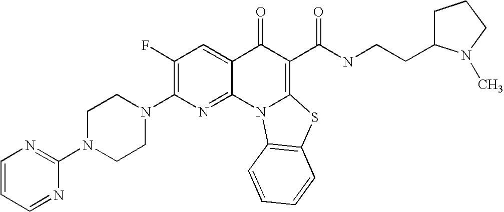

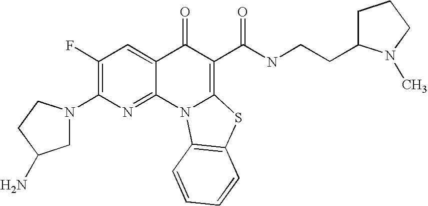

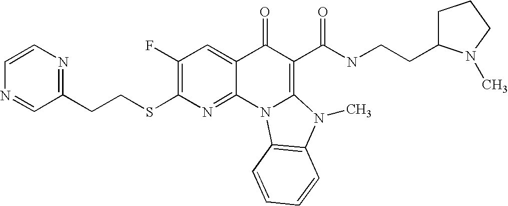

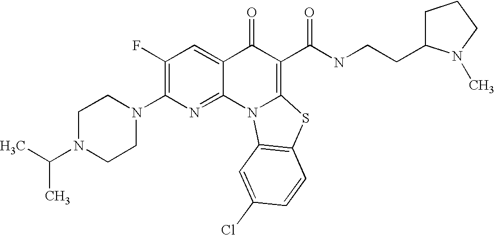

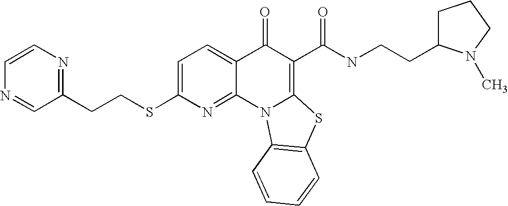

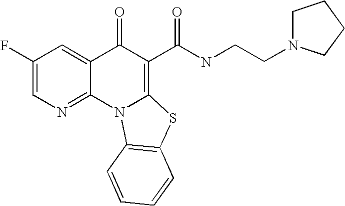

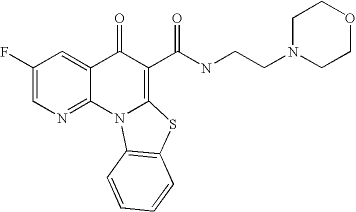

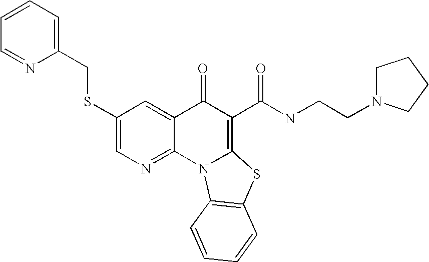

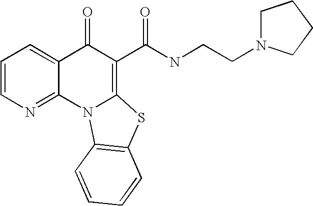

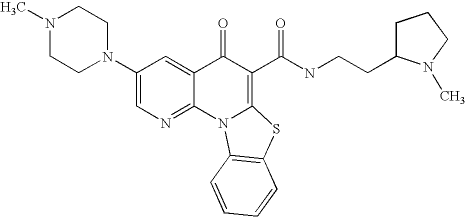

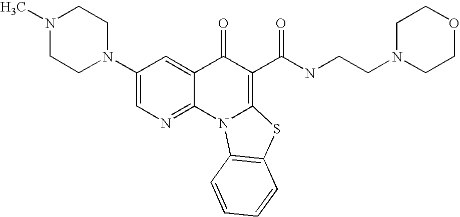

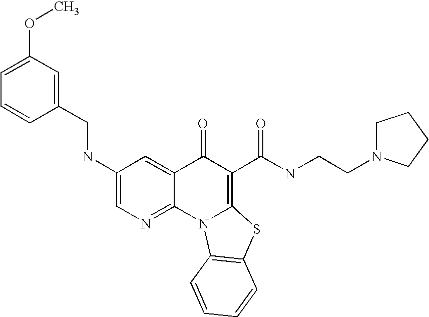

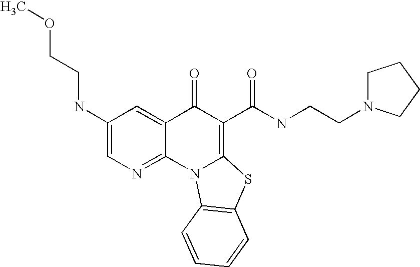

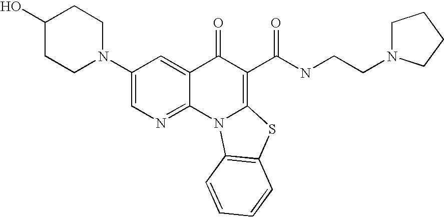

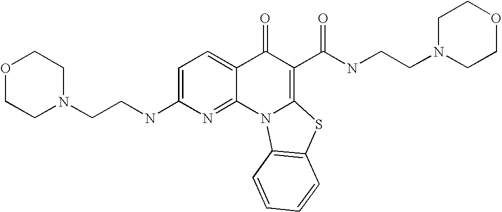

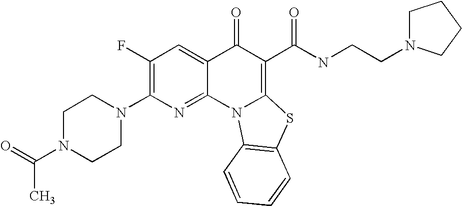

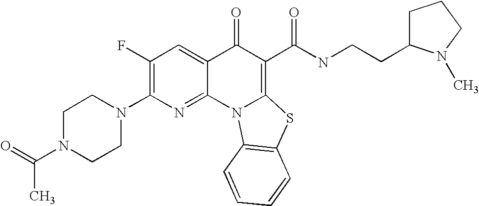

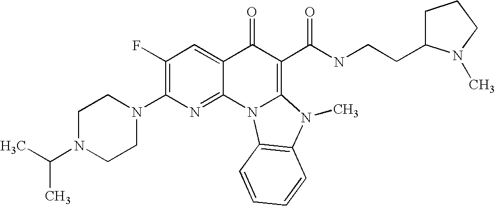

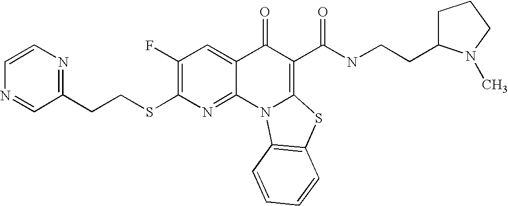

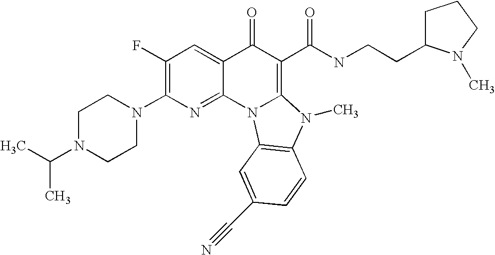

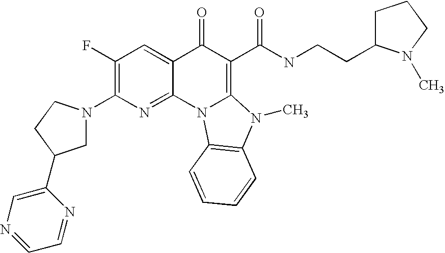

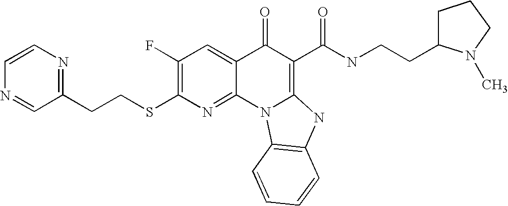

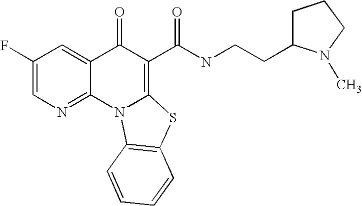

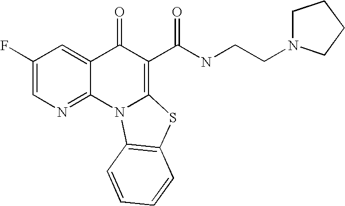

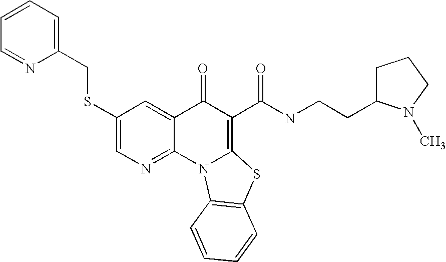

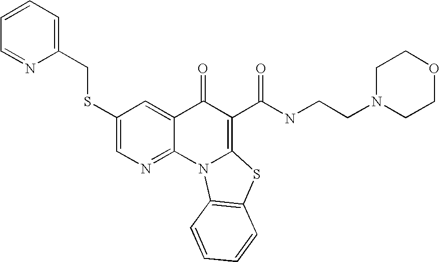

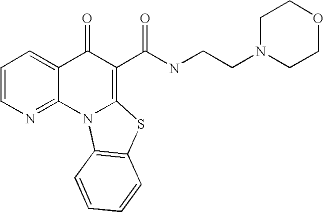

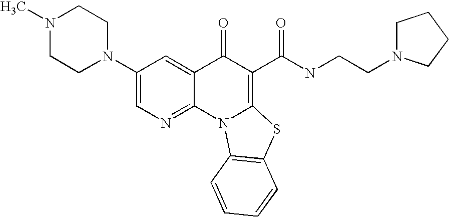

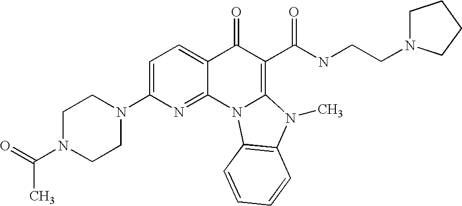

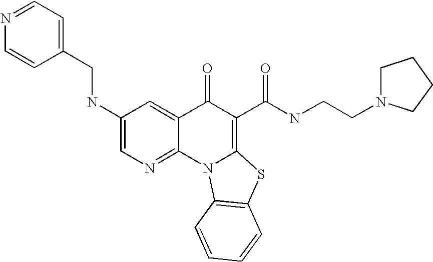

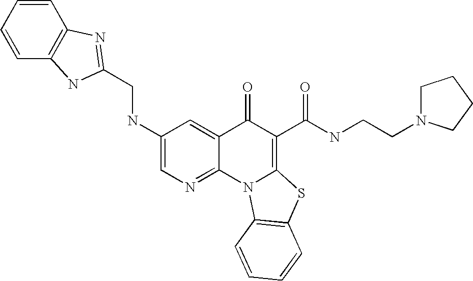

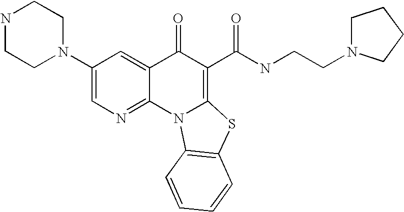

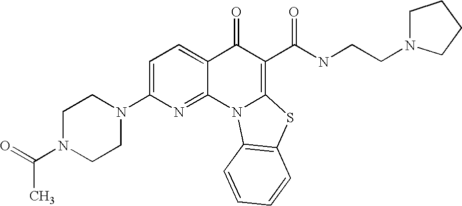

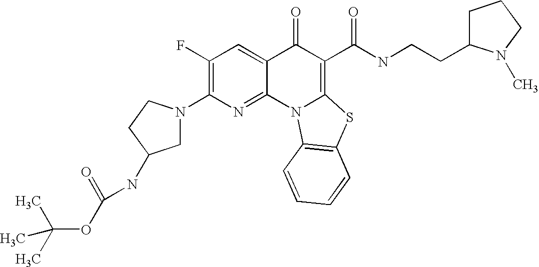

TABLE 1C

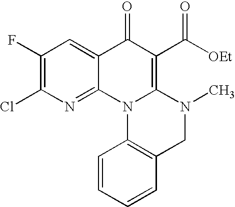

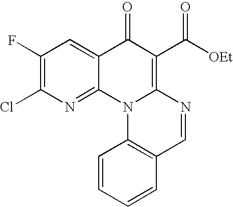

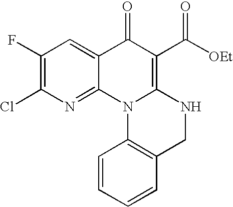

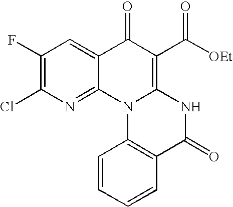

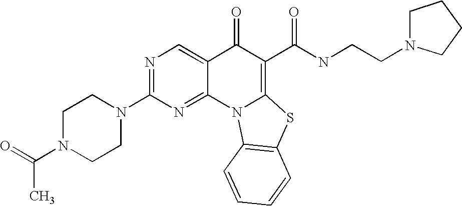

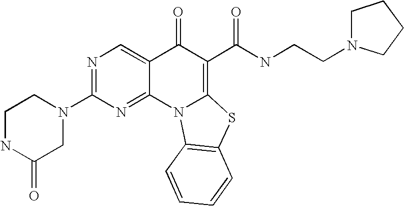

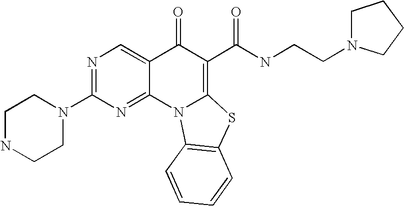

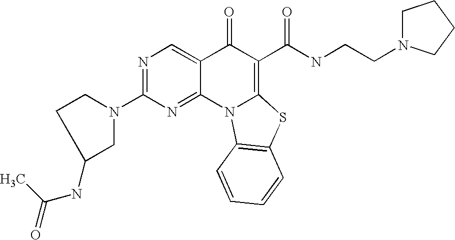

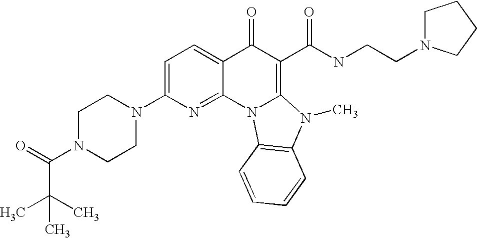

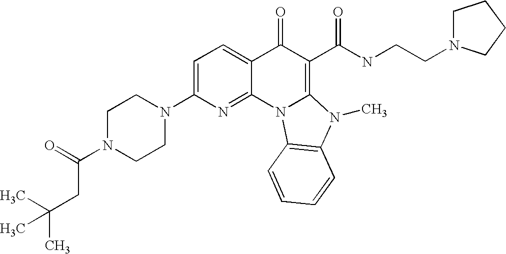

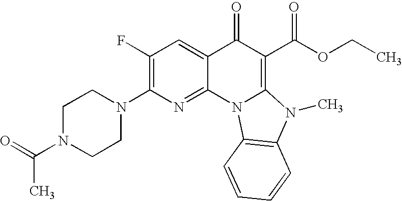

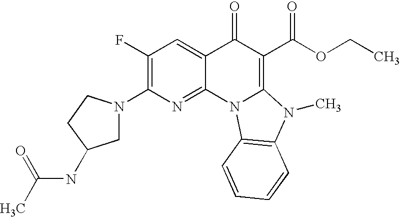

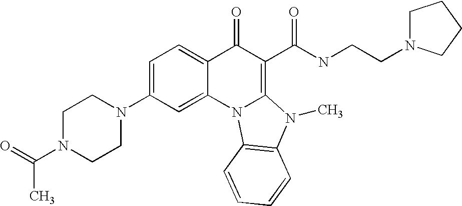

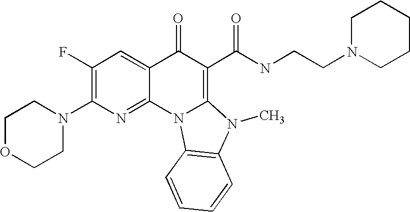

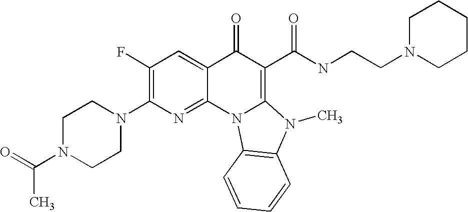

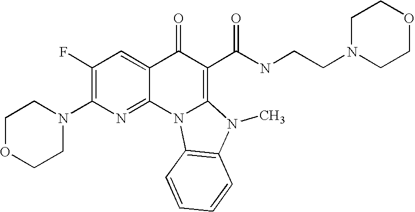

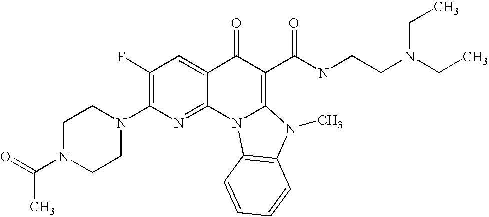

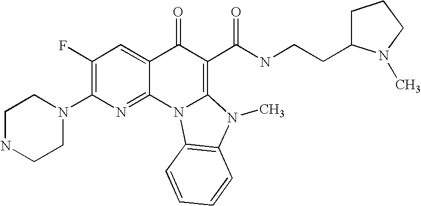

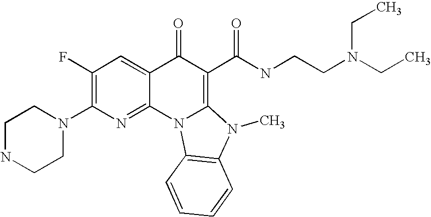

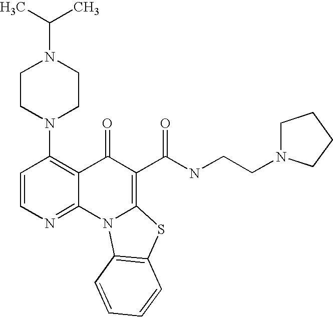

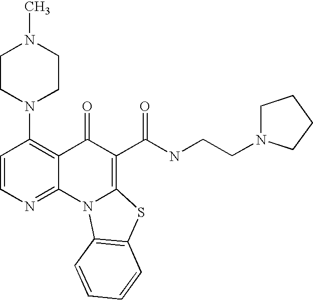

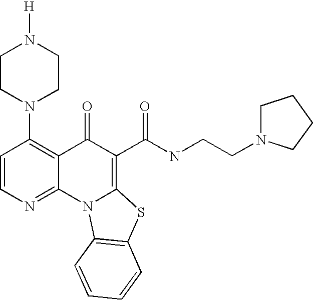

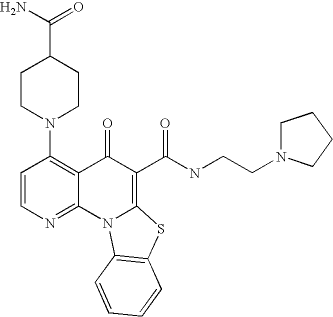

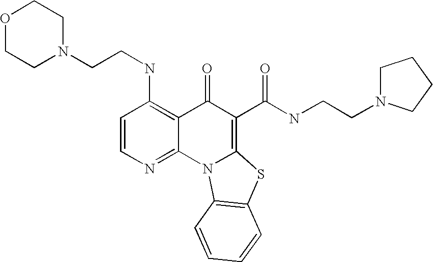

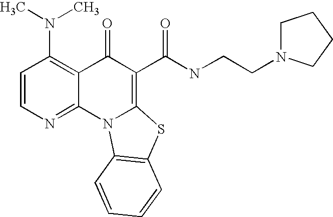

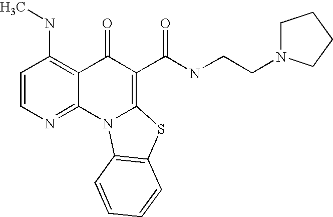

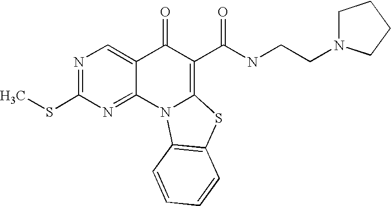

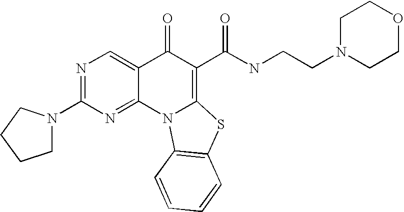

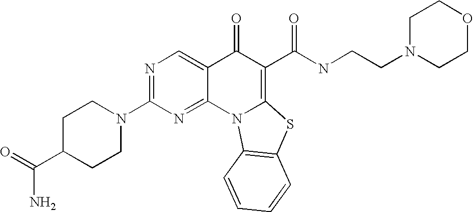

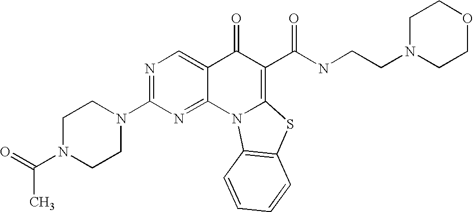

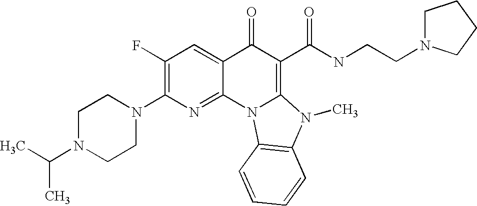

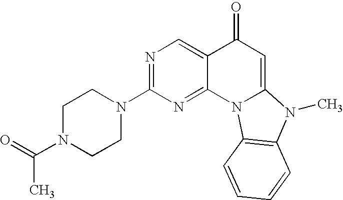

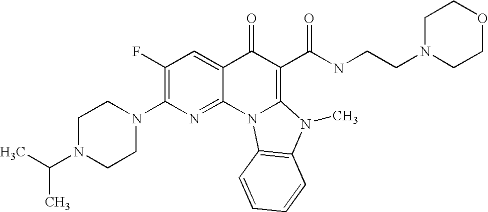

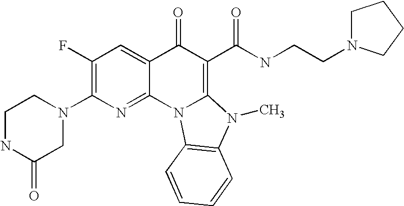

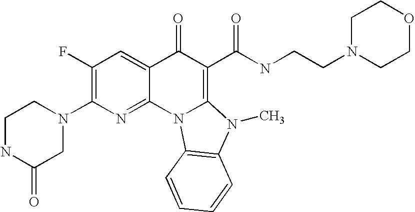

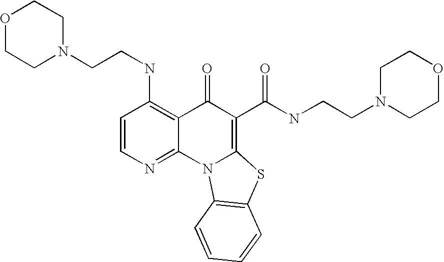

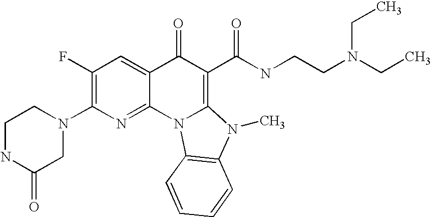

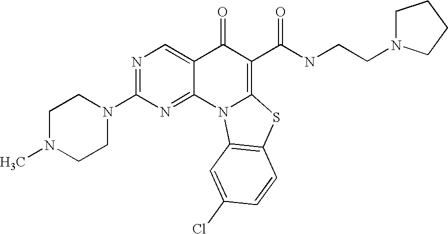

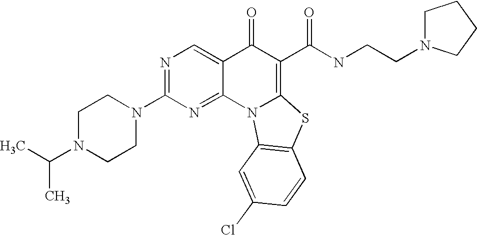

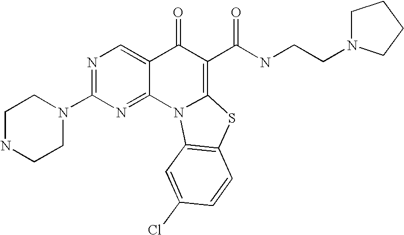

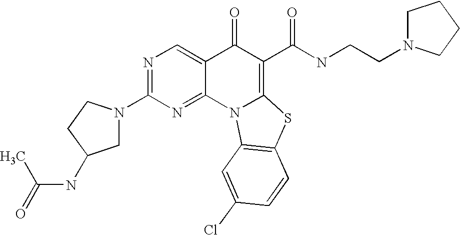

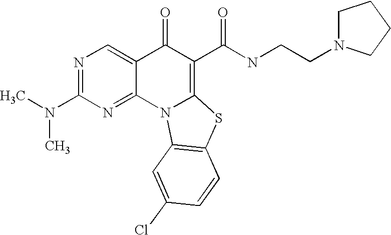

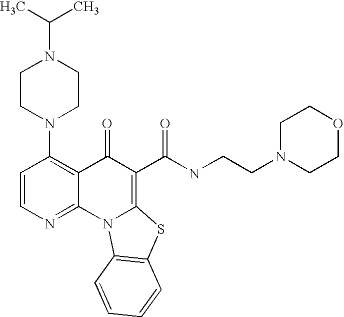

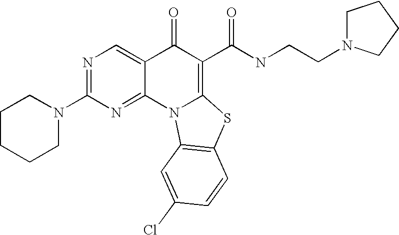

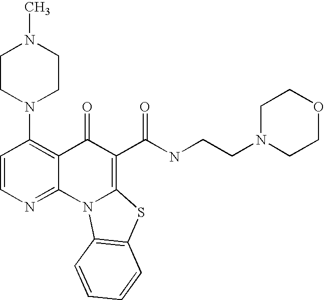

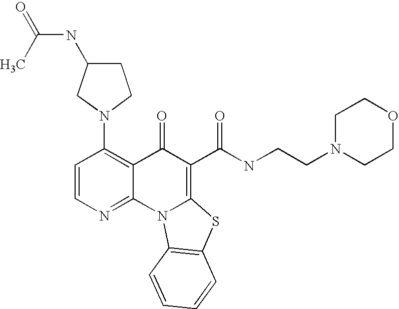

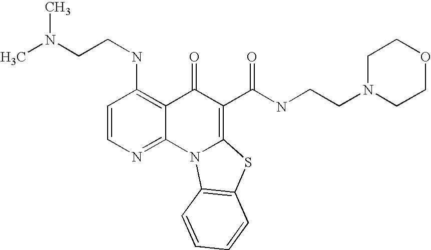

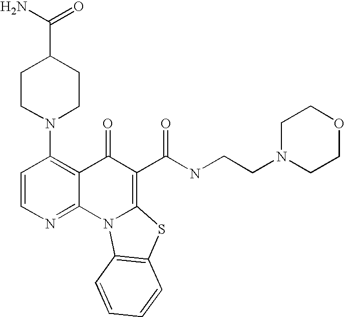

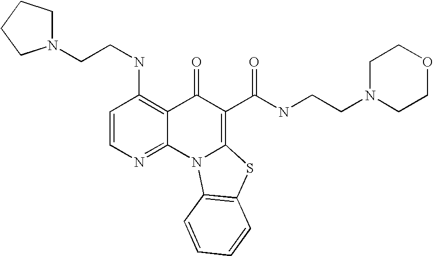

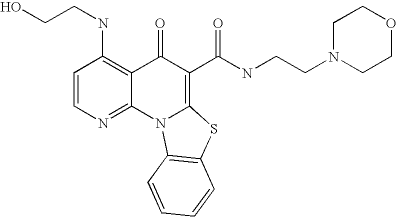

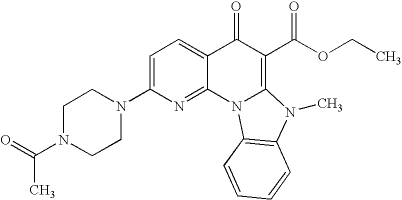

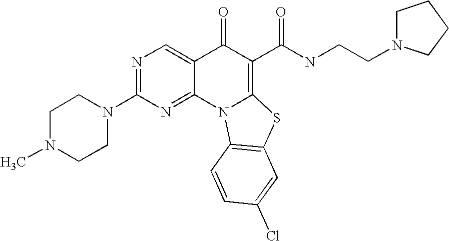

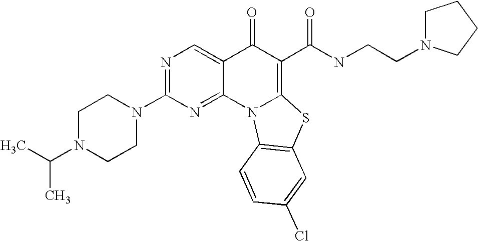

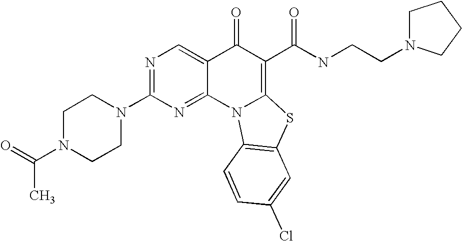

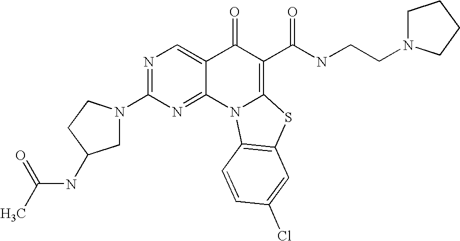

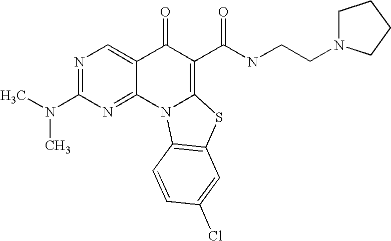

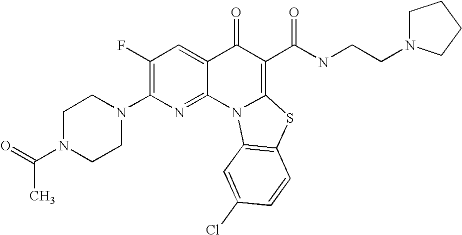

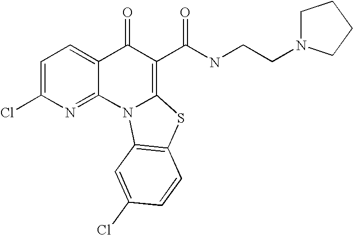

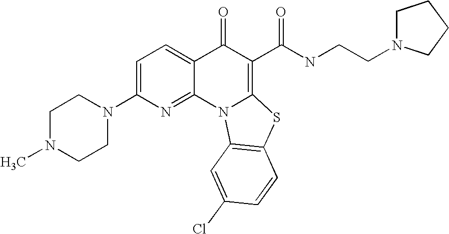

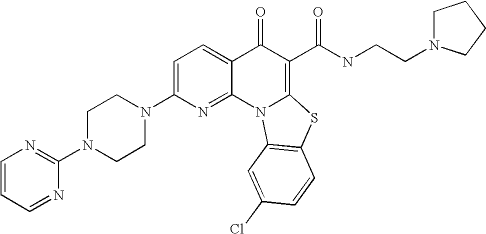

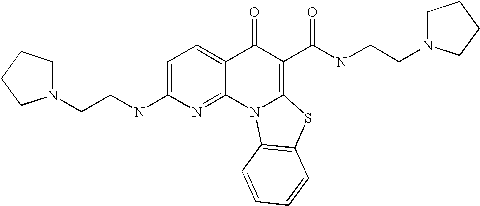

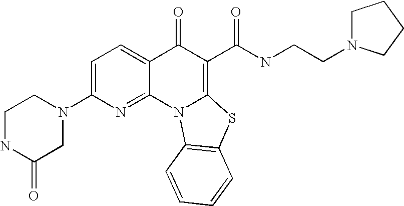

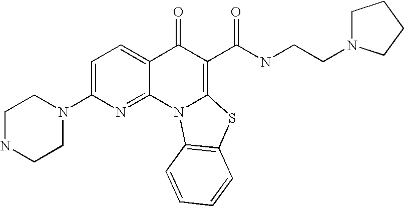

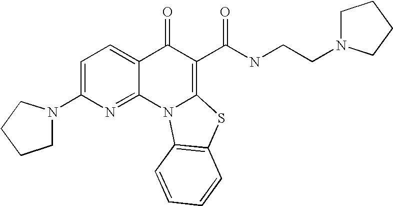

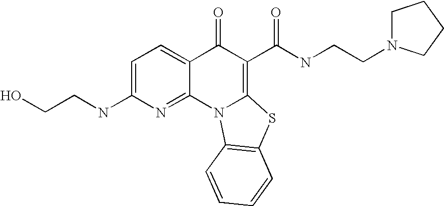

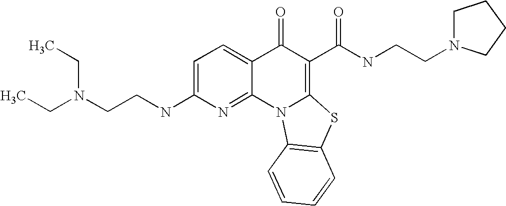

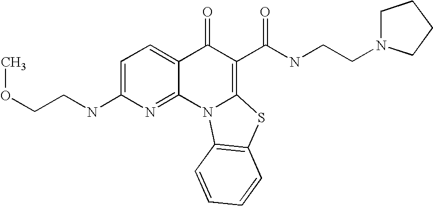

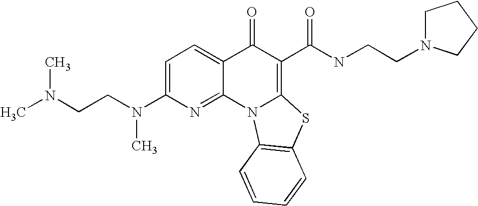

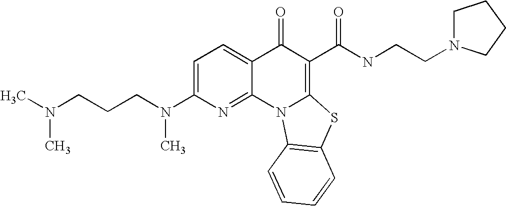

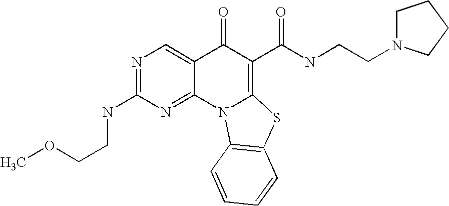

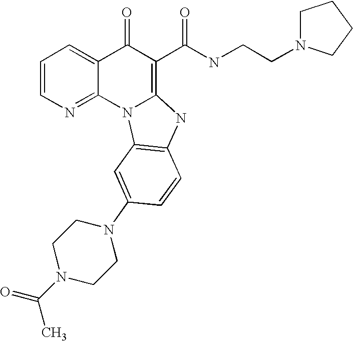

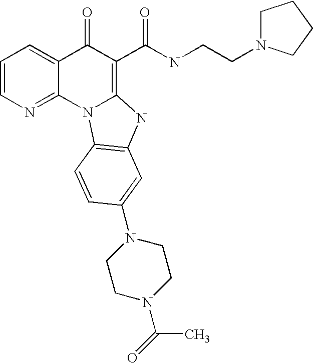

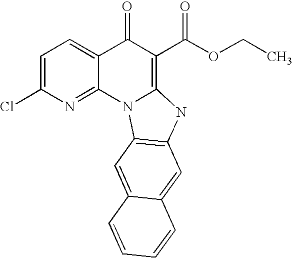

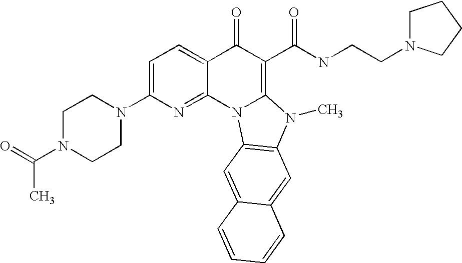

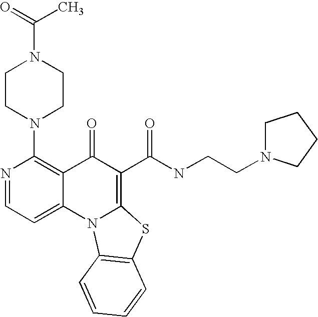

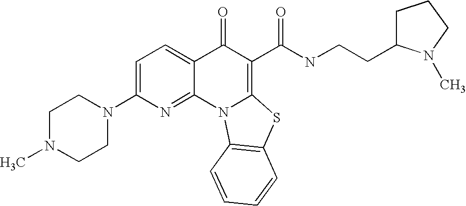

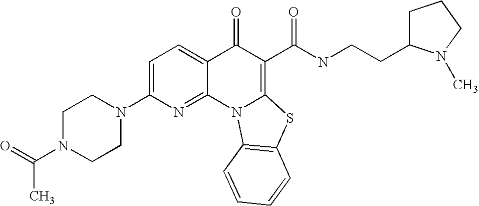

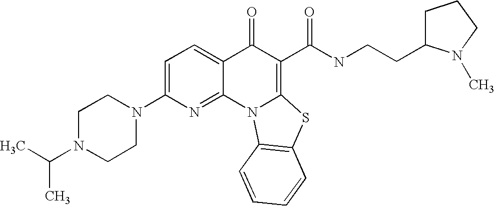

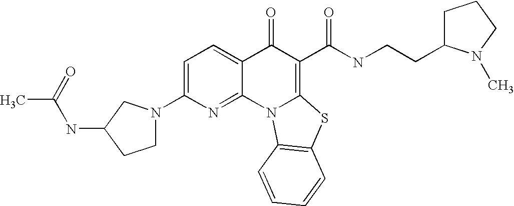

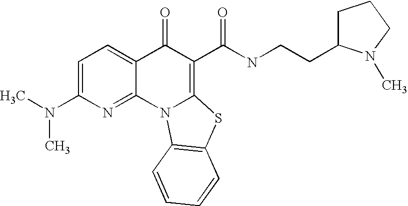

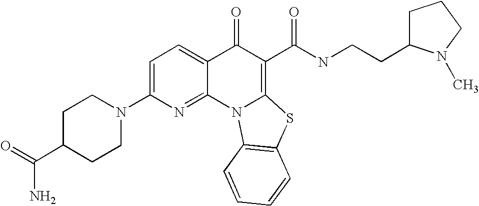

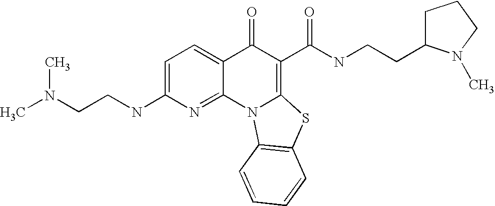

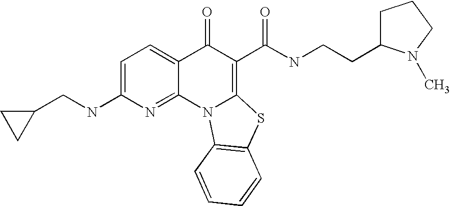

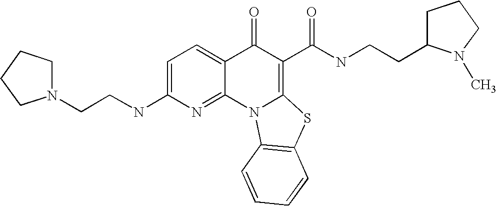

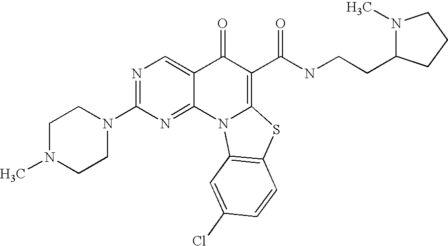

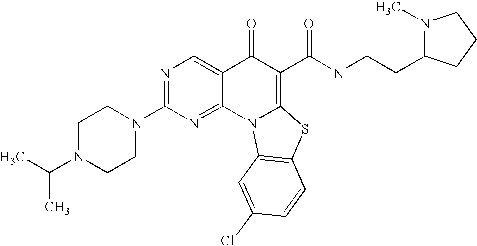

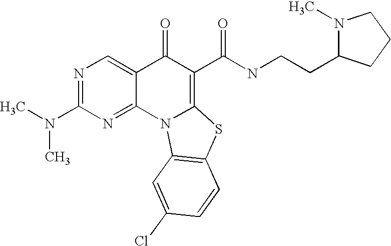

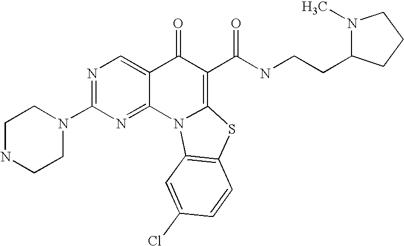

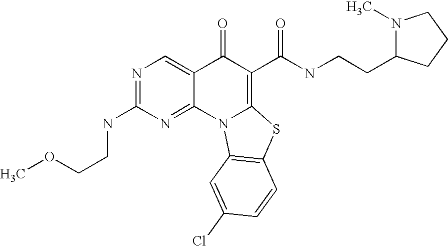

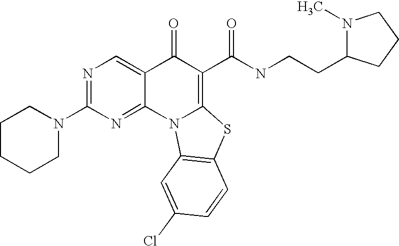

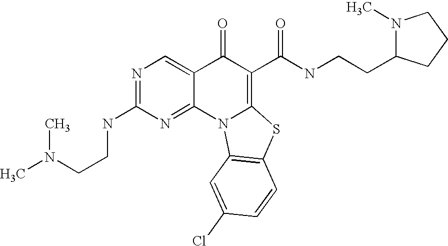

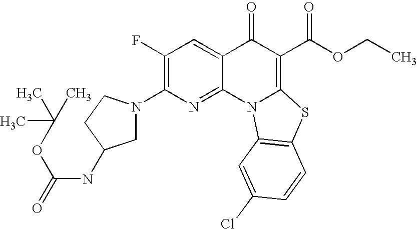

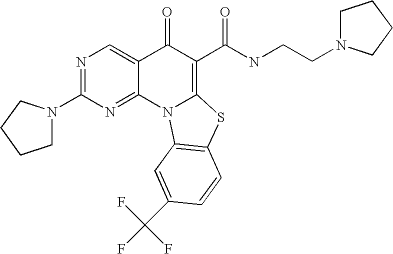

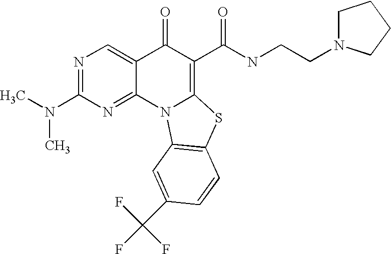

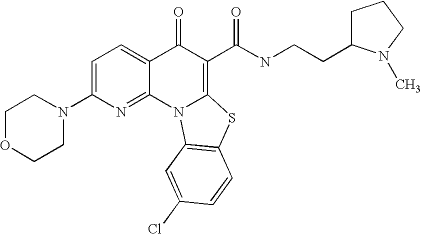

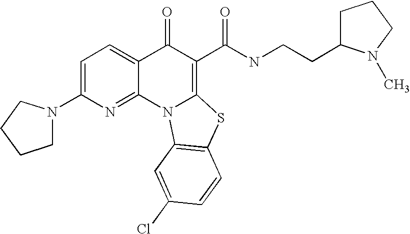

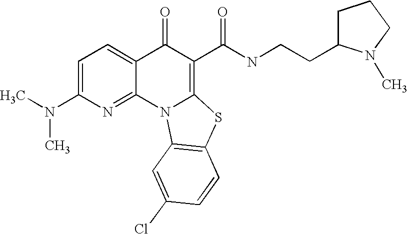

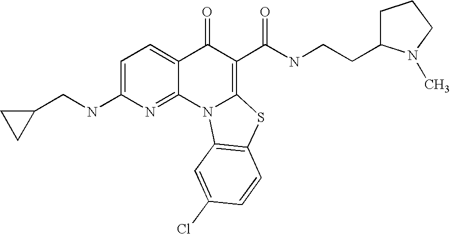

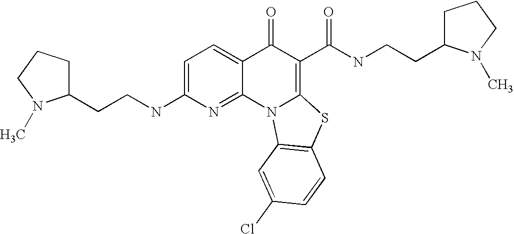

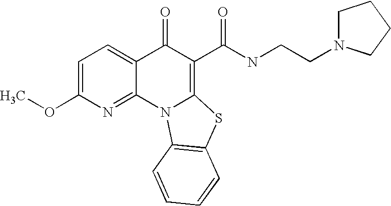

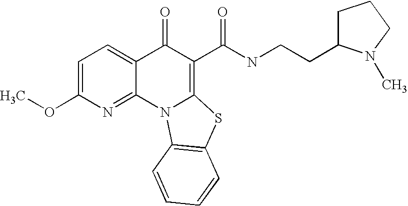

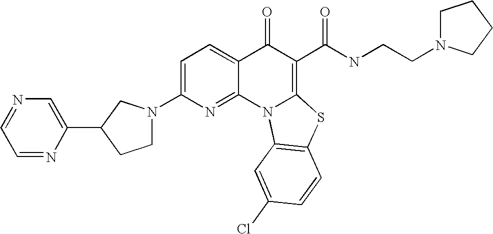

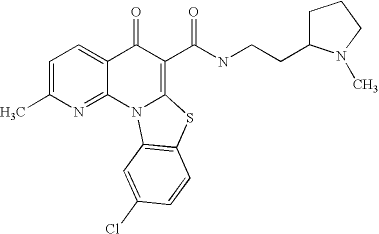

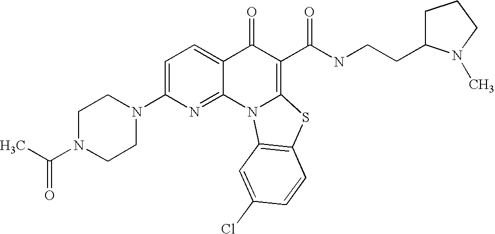

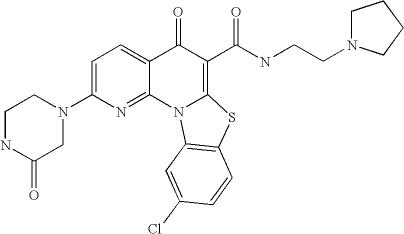

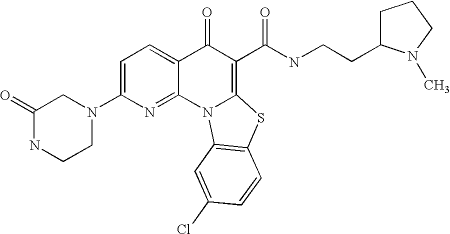

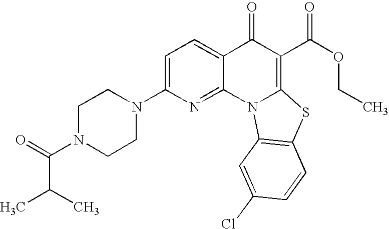

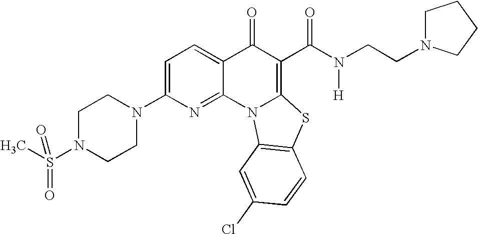

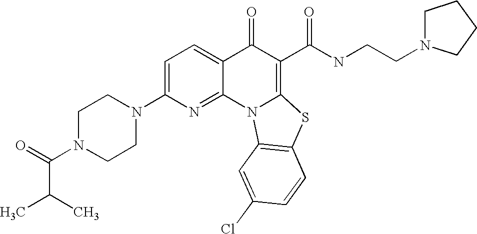

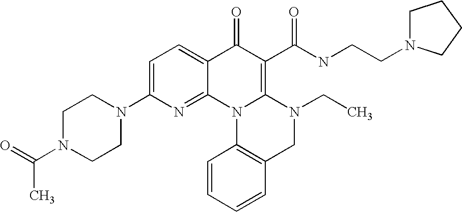

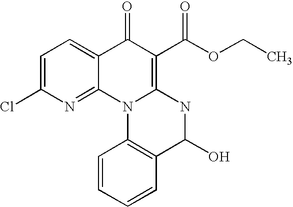

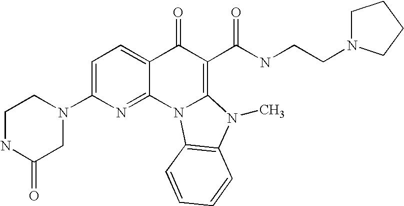

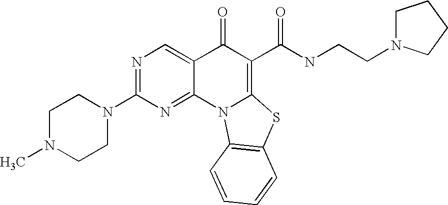

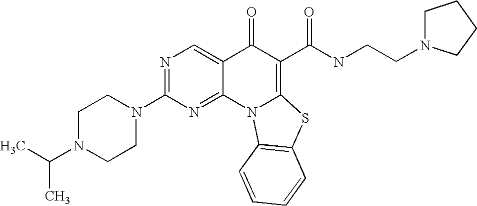

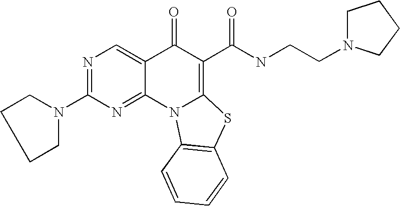

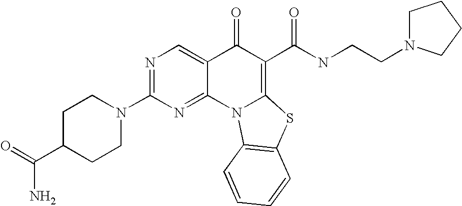

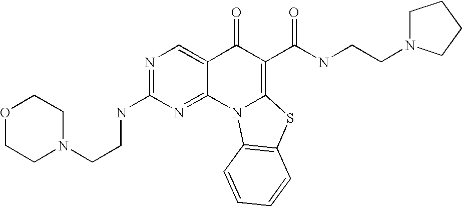

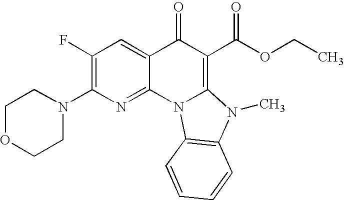

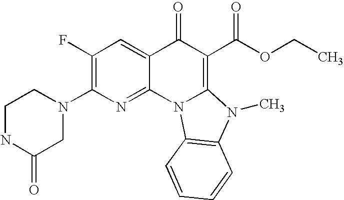

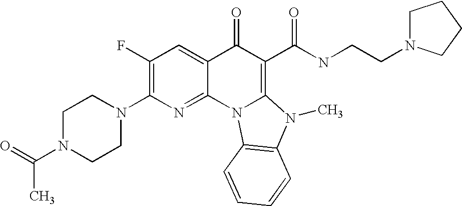

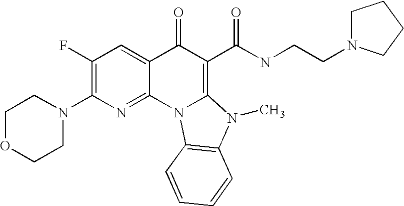

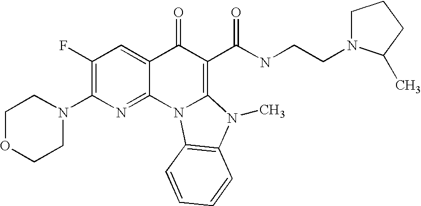

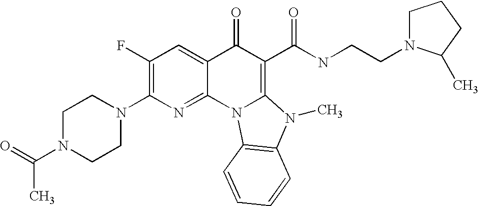

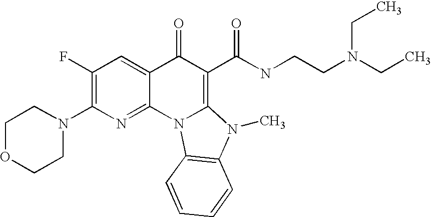

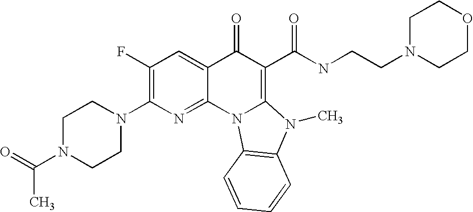

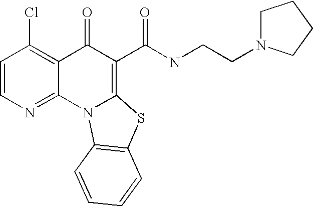

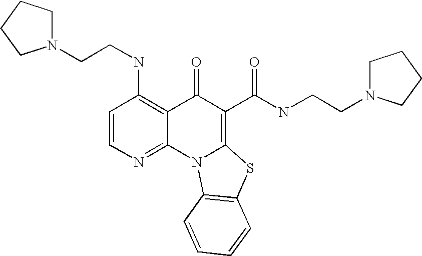

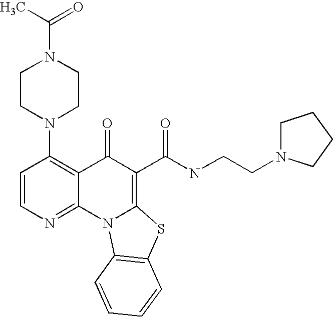

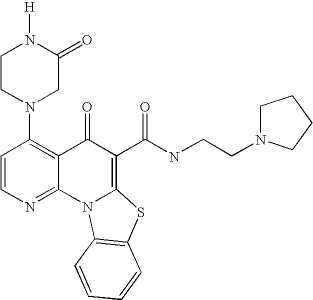

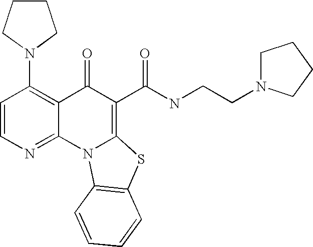

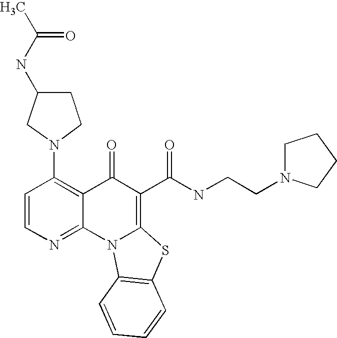

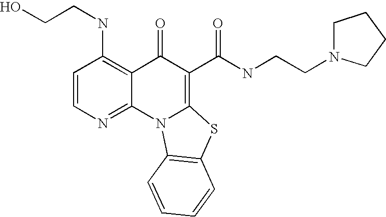

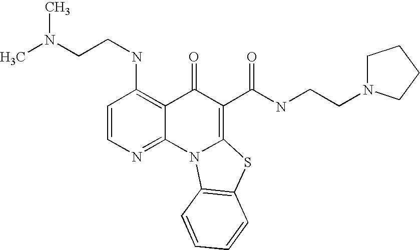

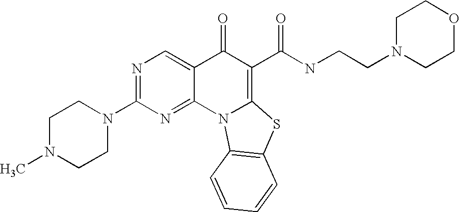

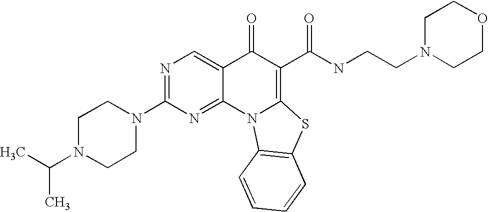

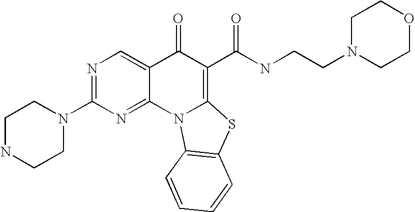

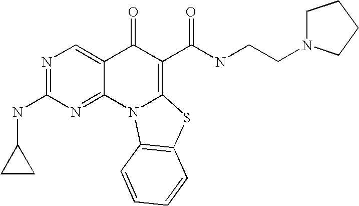

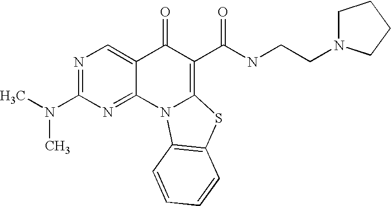

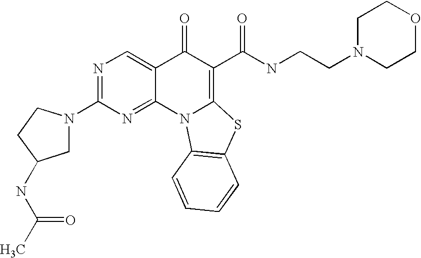

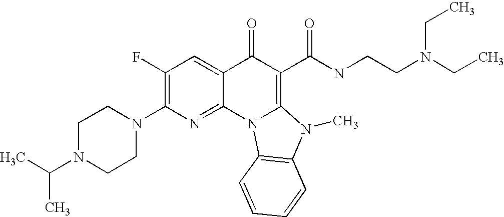

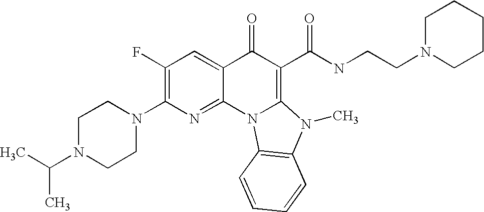

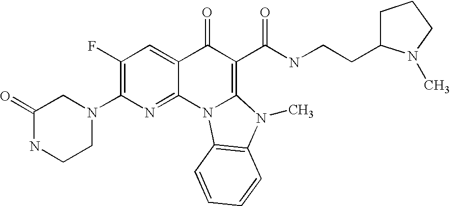

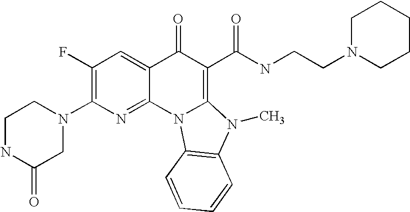

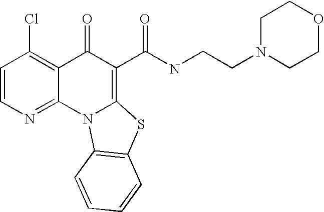

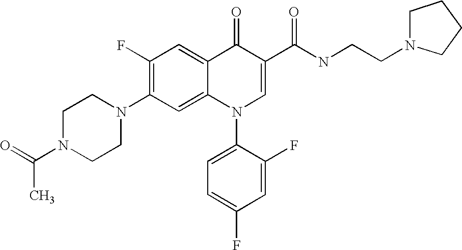

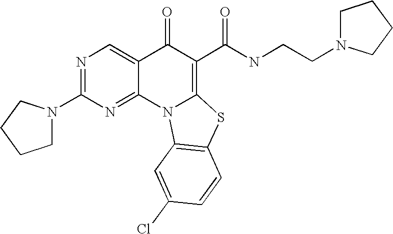

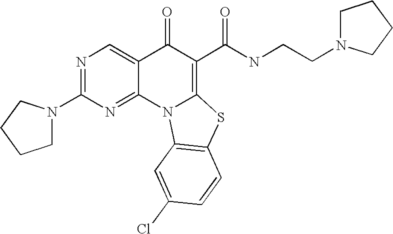

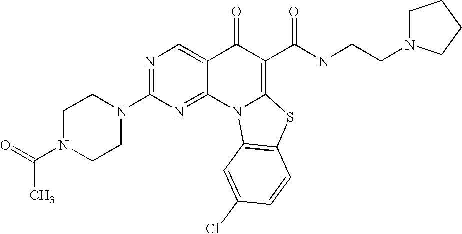

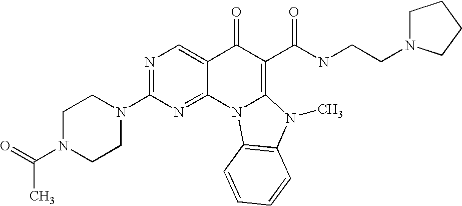

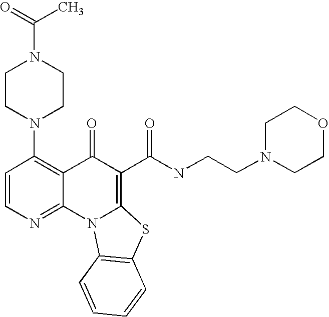

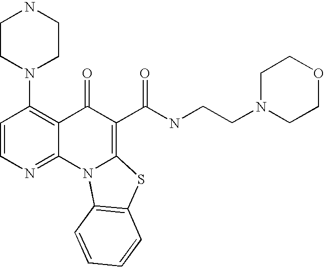

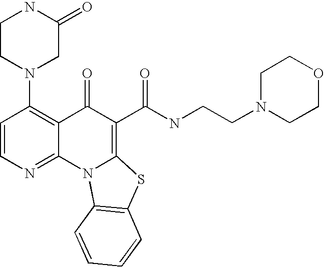

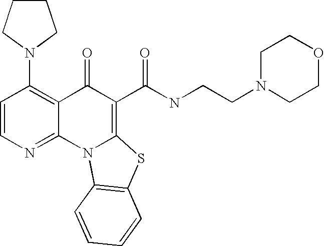

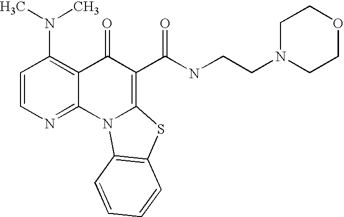

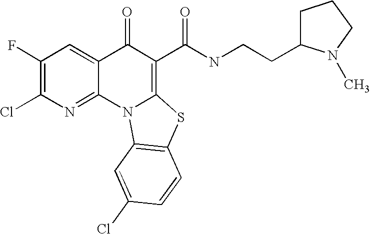

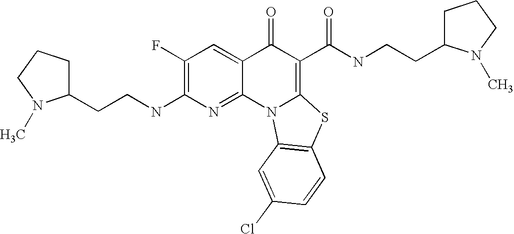

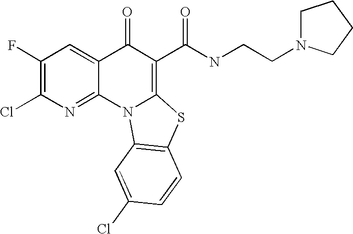

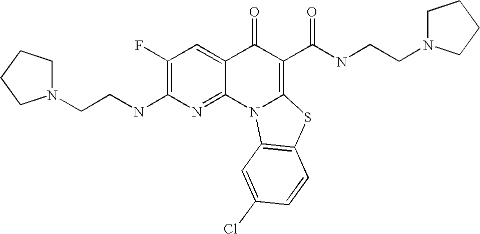

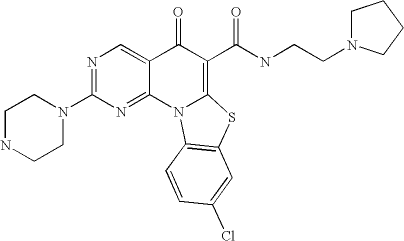

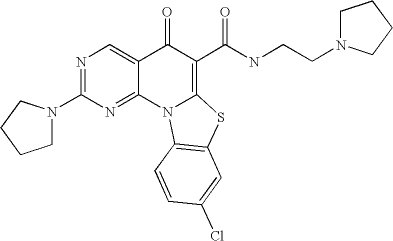

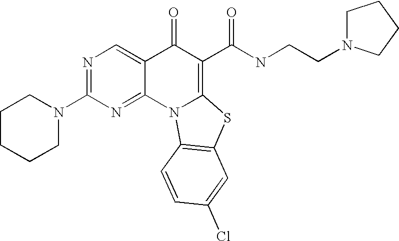

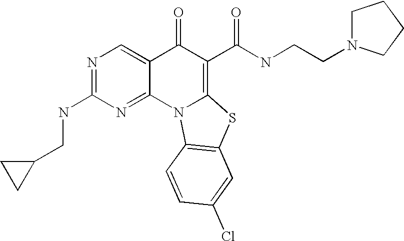

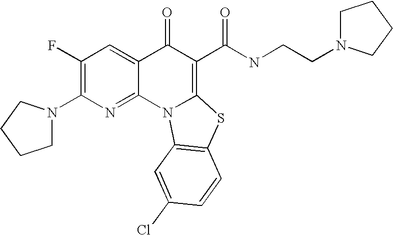

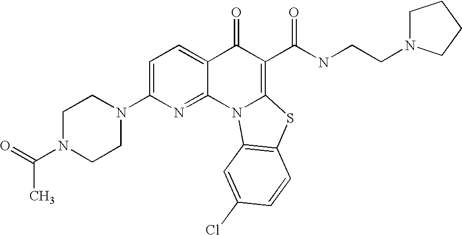

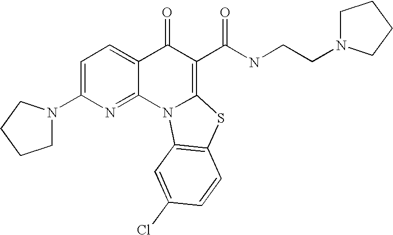

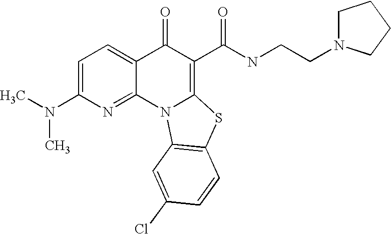

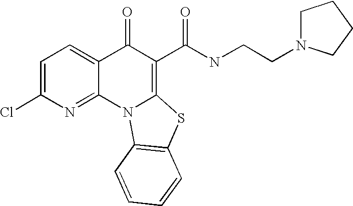

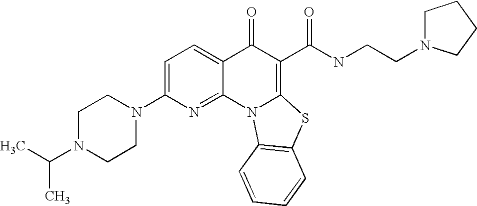

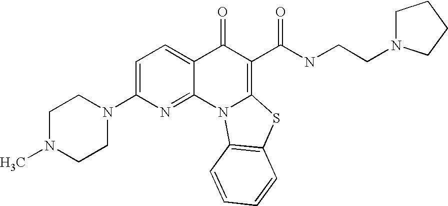

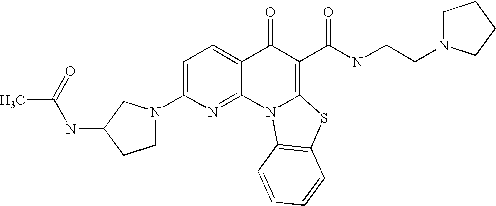

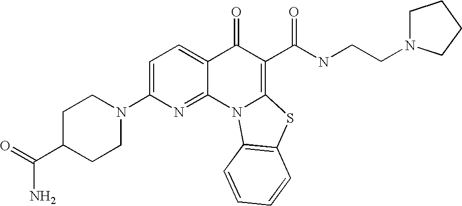

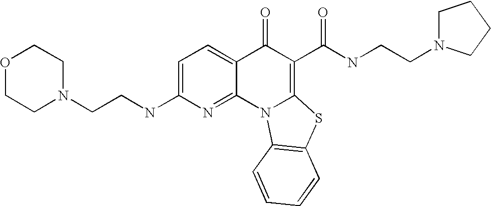

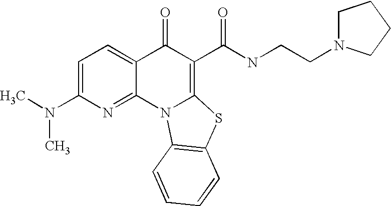

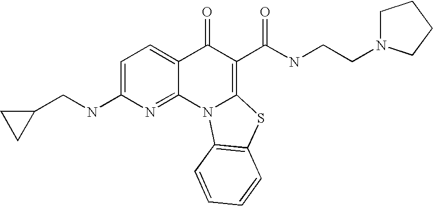

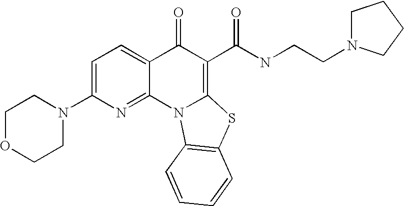

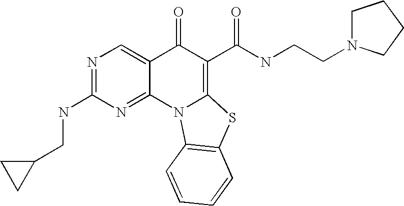

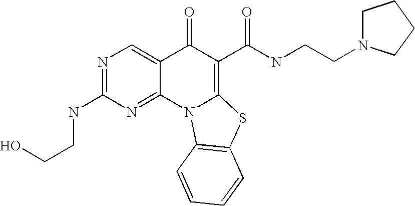

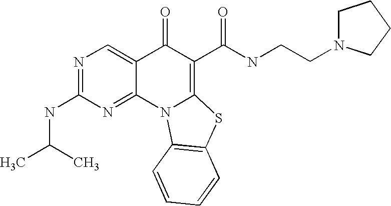

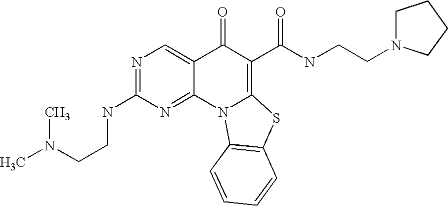

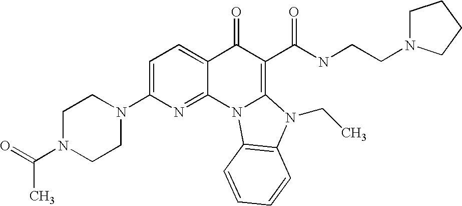

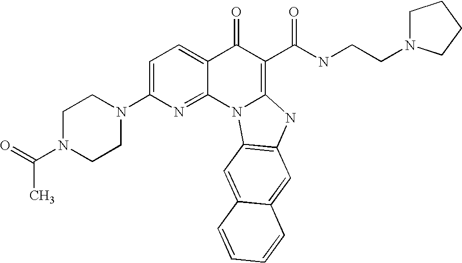

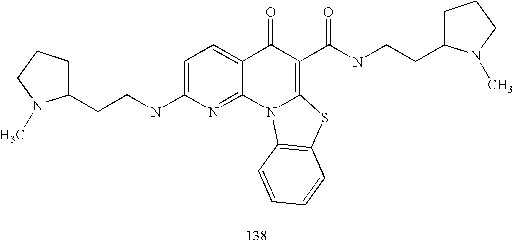

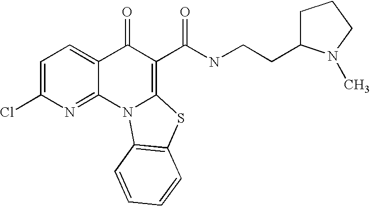

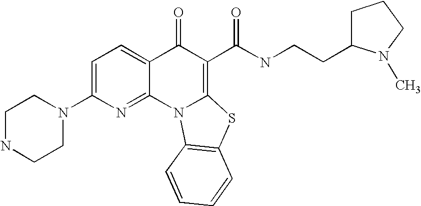

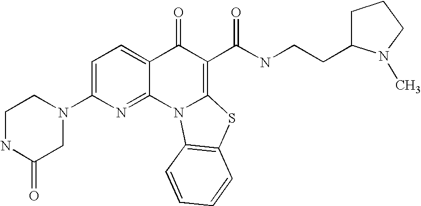

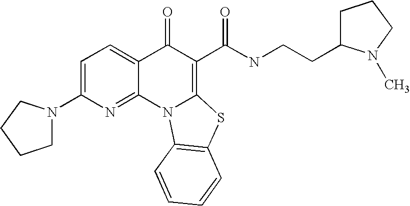

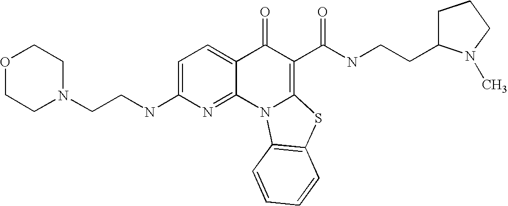

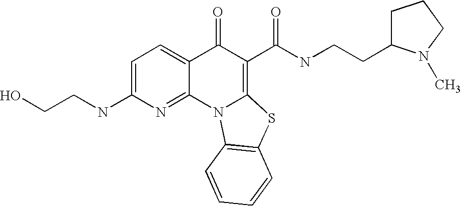

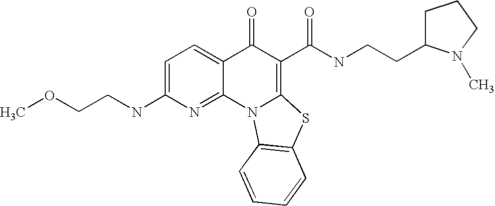

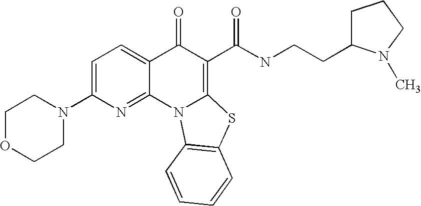

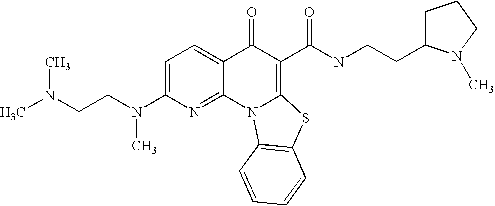

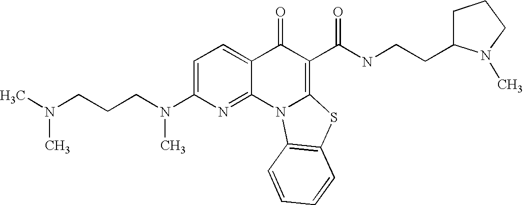

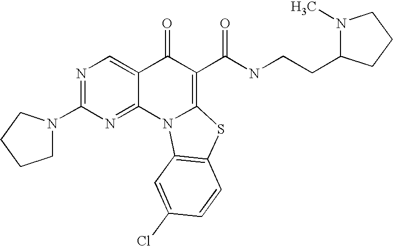

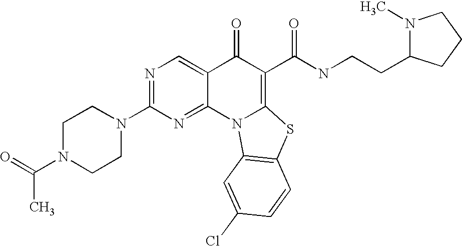

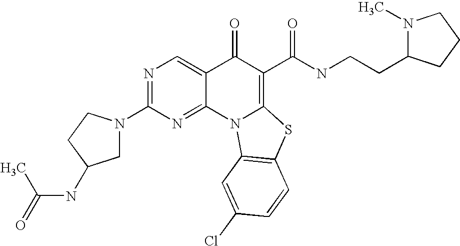

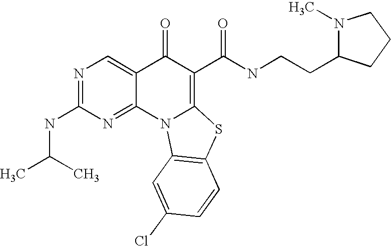

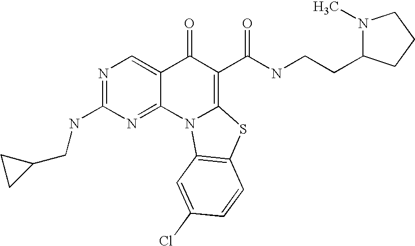

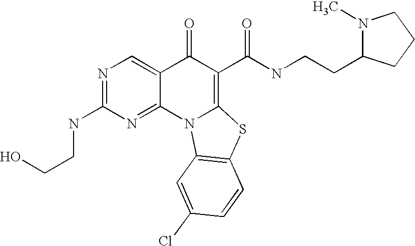

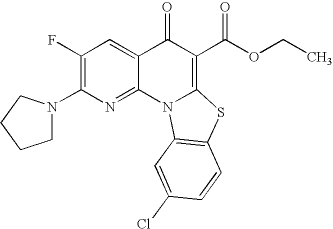

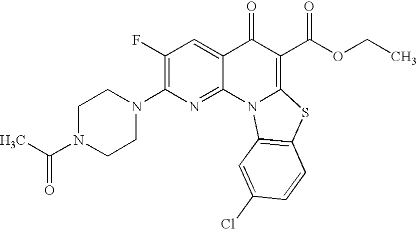

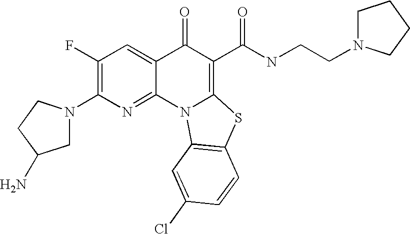

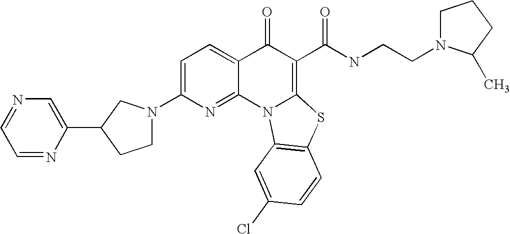

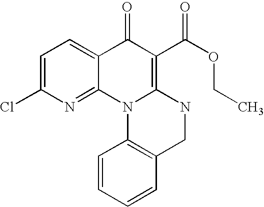

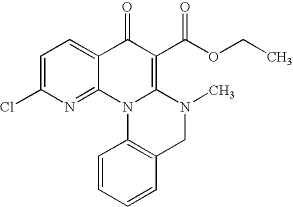

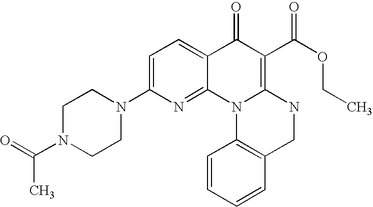

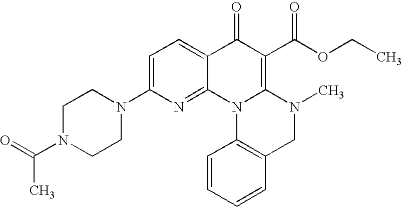

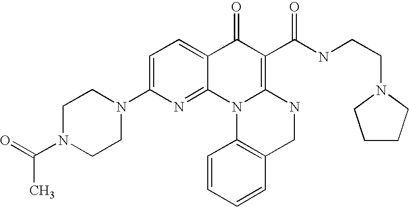

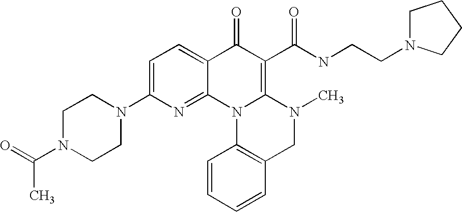

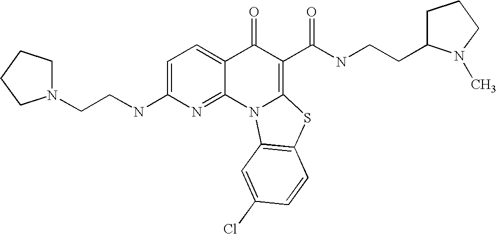

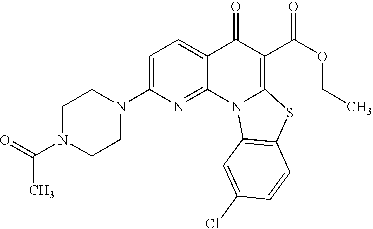

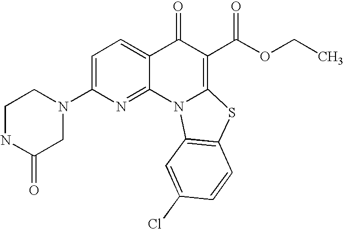

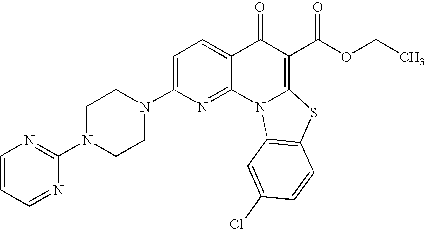

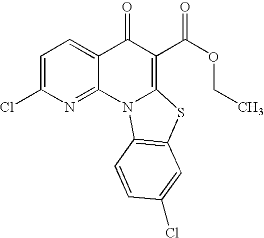

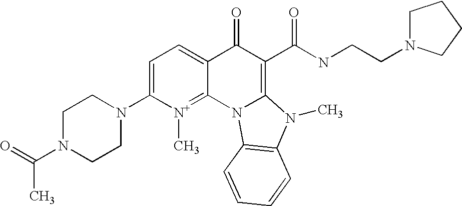

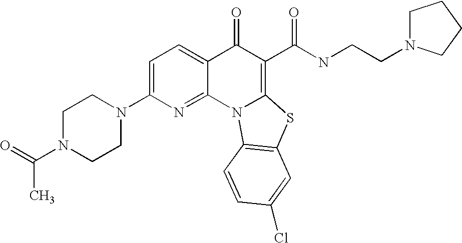

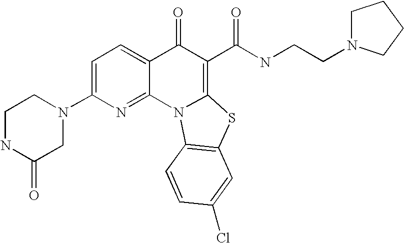

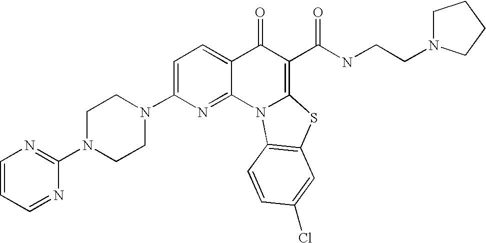

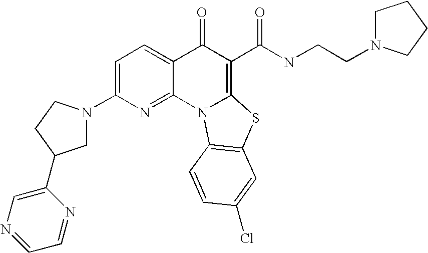

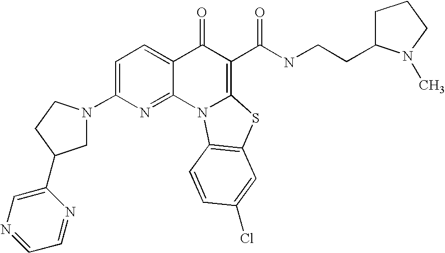

TABLE 2

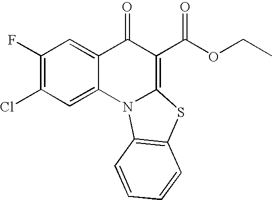

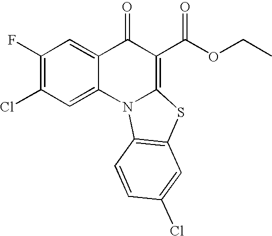

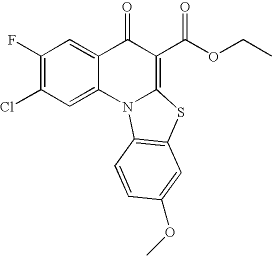

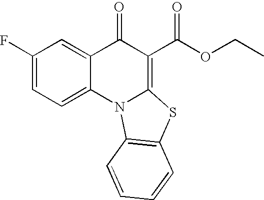

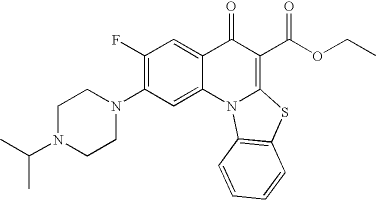

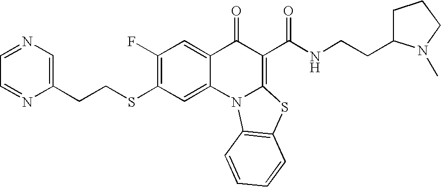

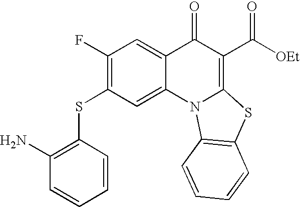

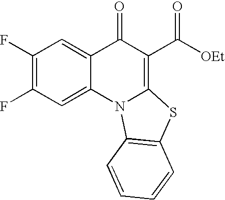

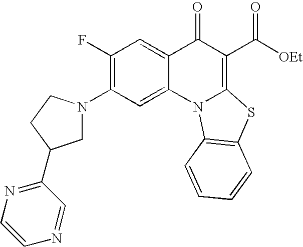

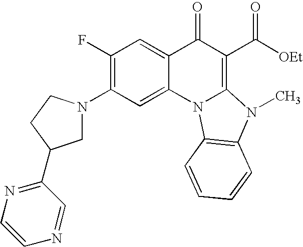

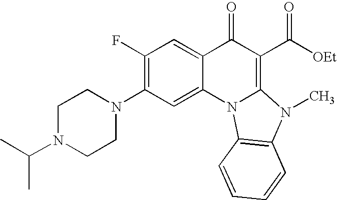

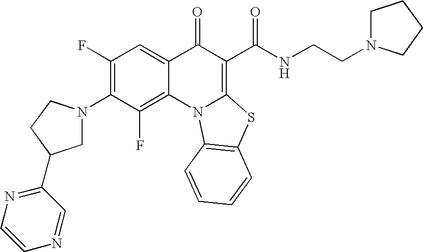

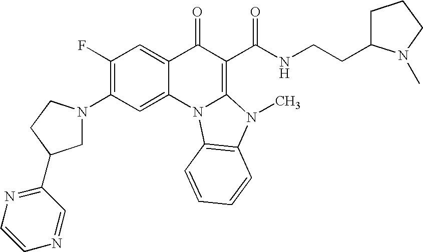

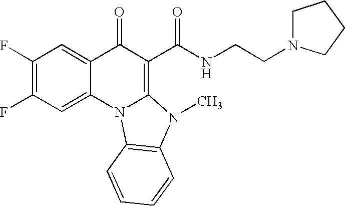

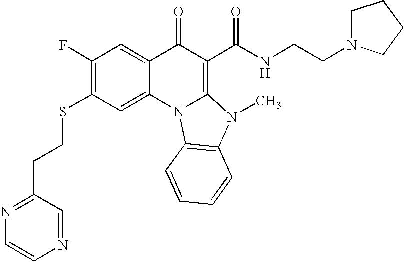

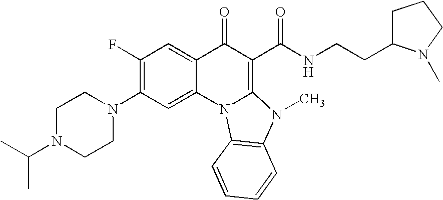

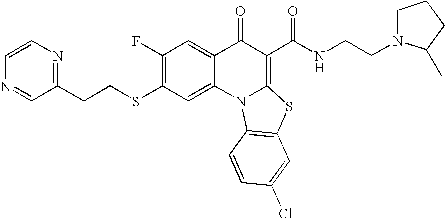

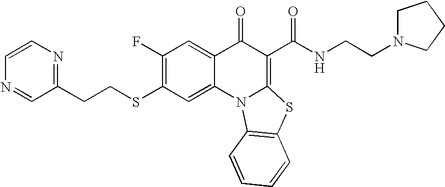

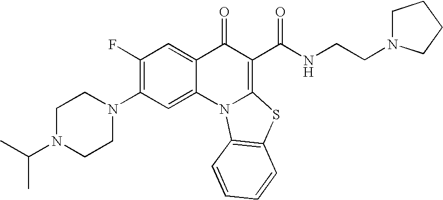

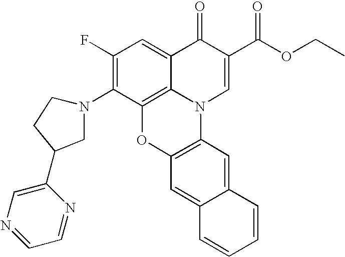

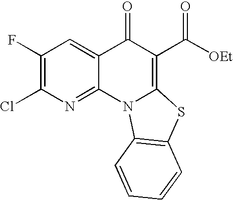

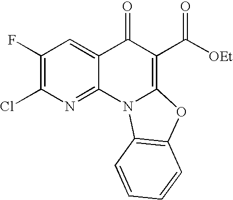

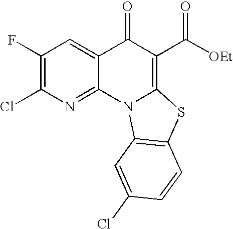

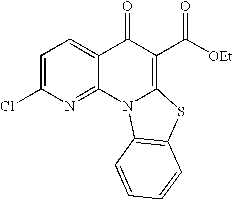

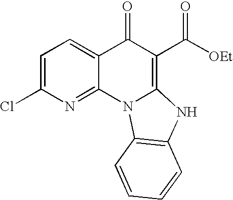

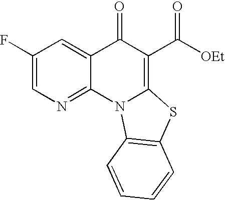

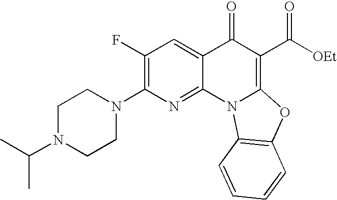

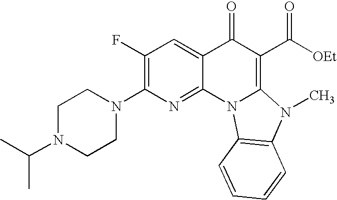

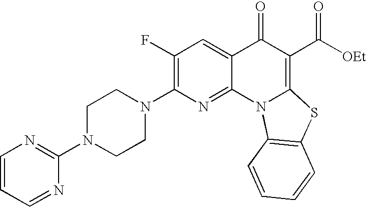

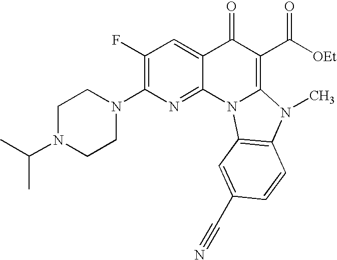

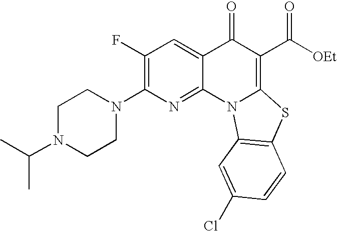

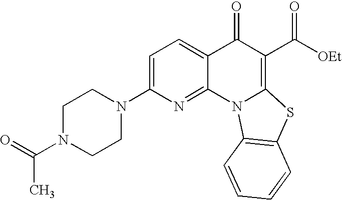

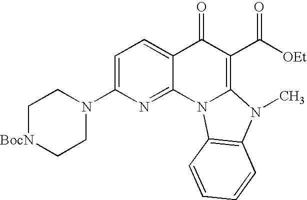

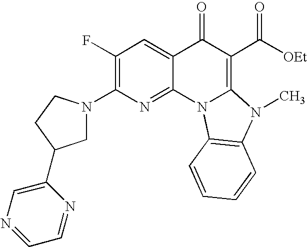

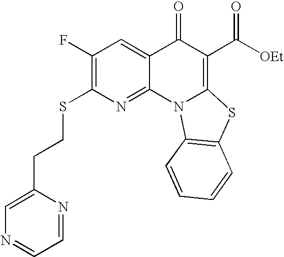

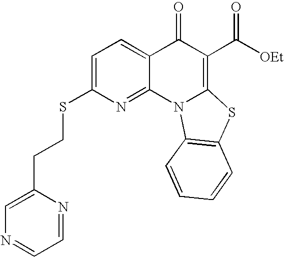

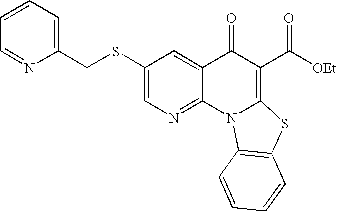

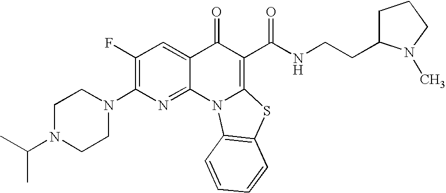

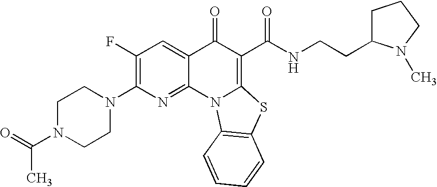

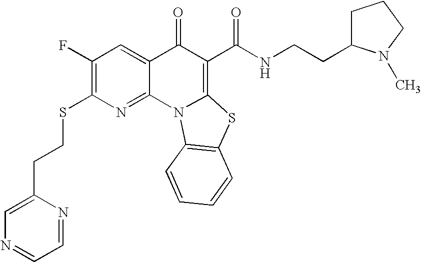

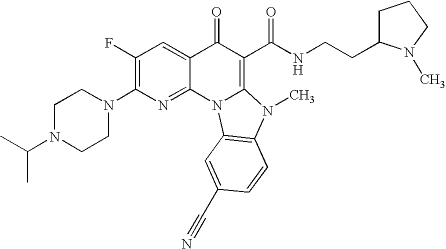

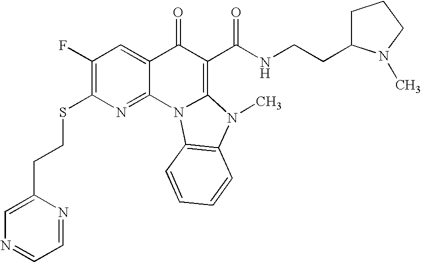

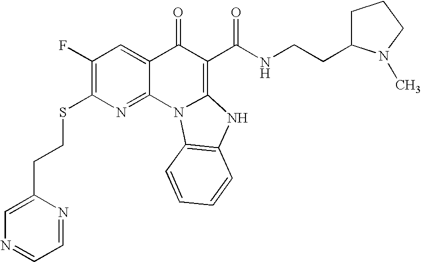

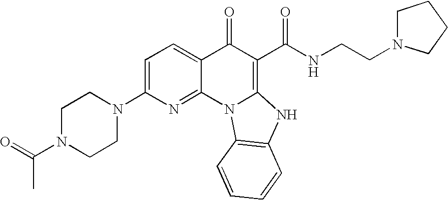

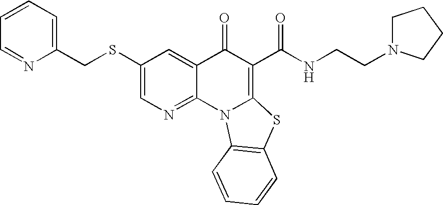

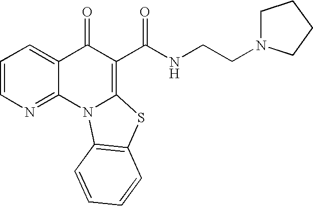

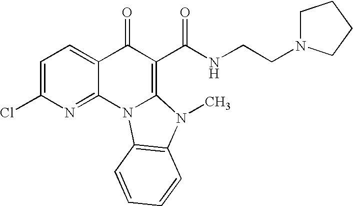

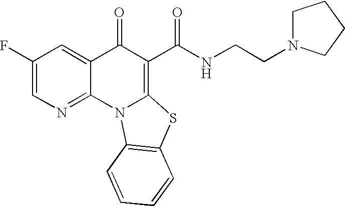

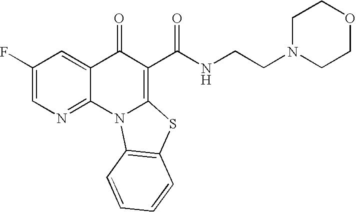

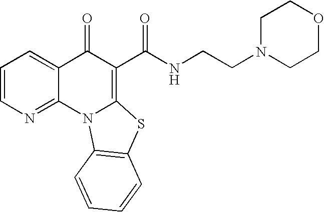

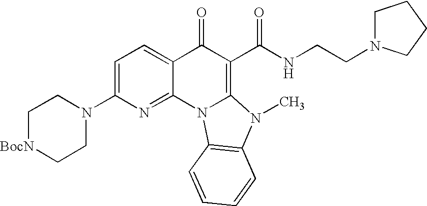

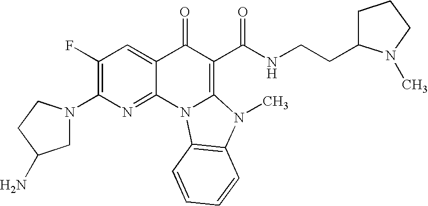

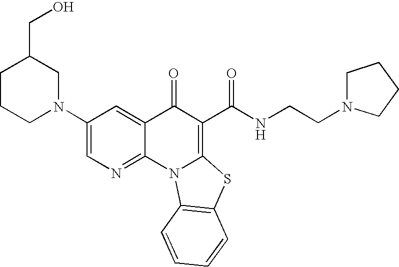

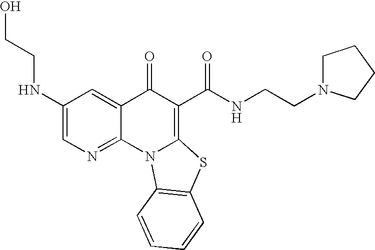

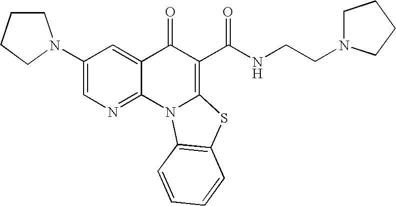

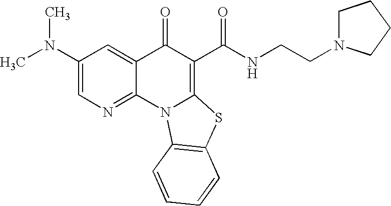

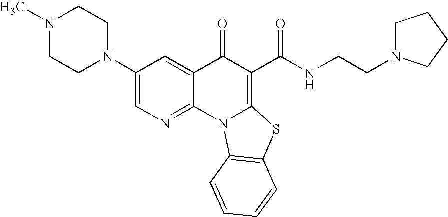

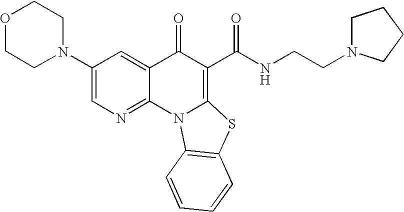

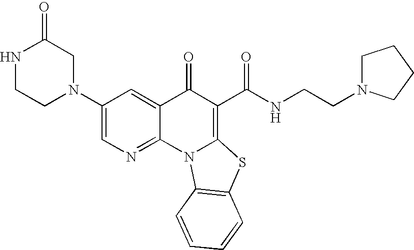

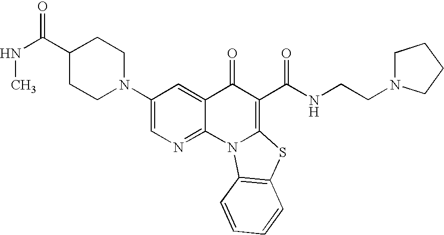

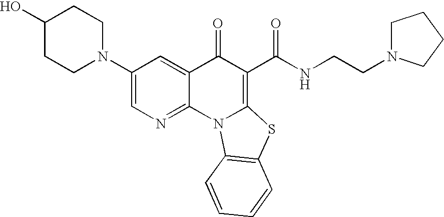

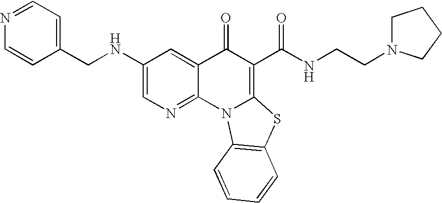

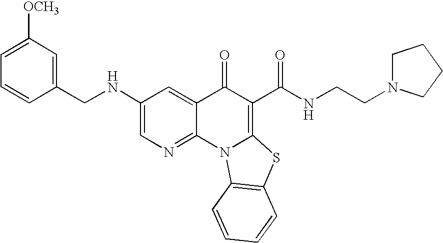

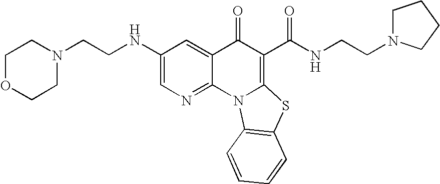

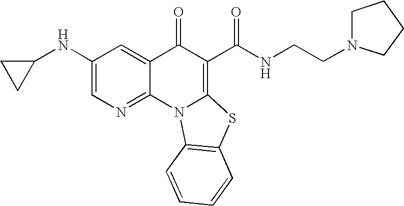

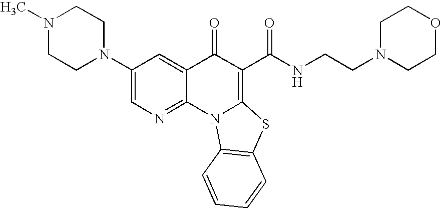

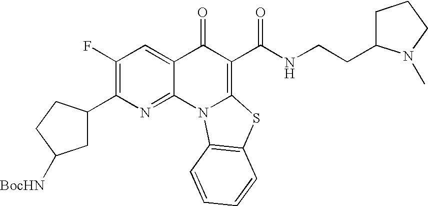

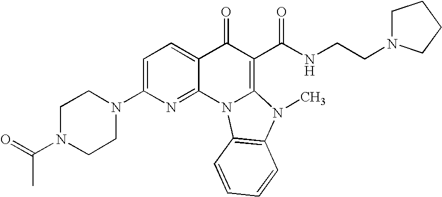

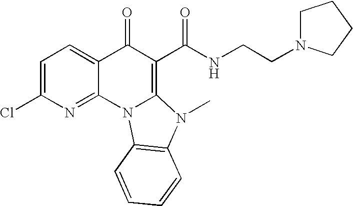

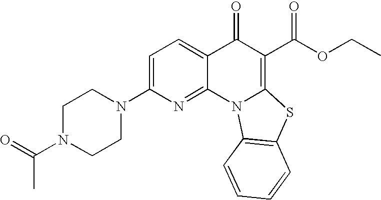

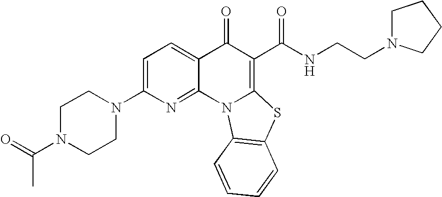

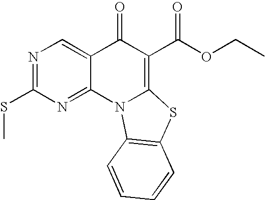

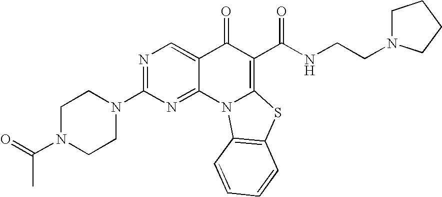

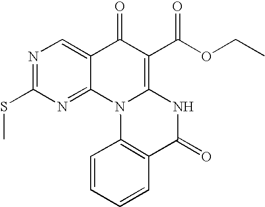

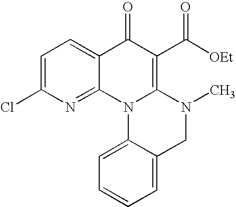

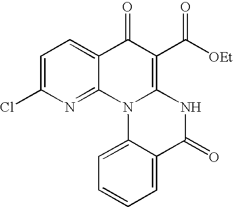

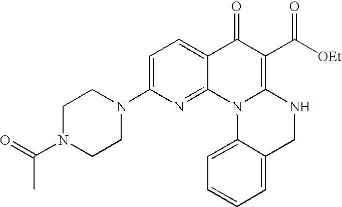

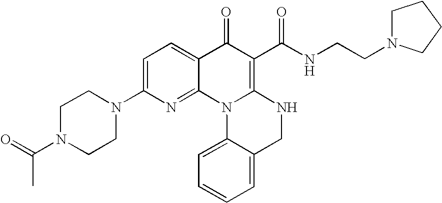

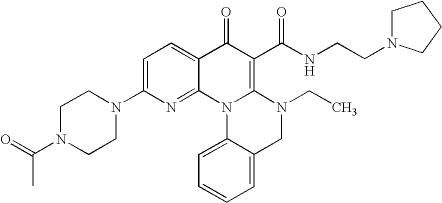

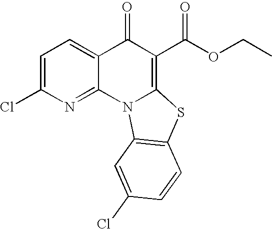

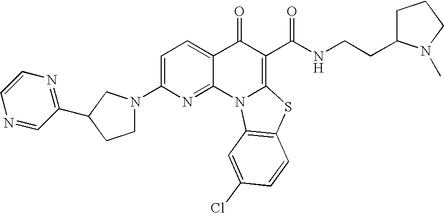

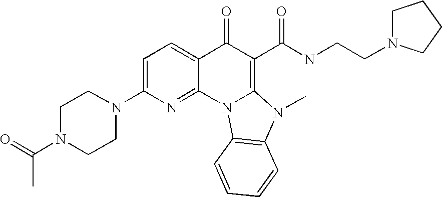

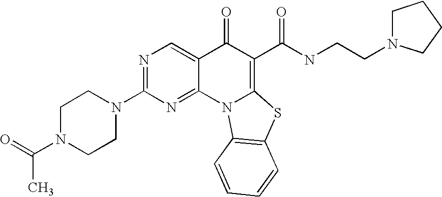

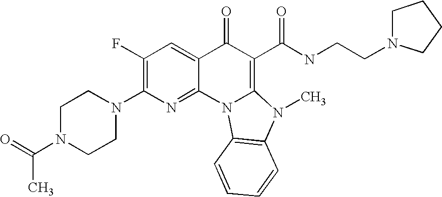

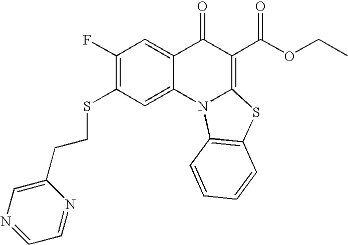

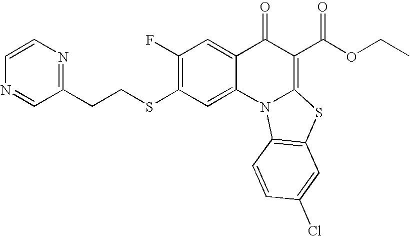

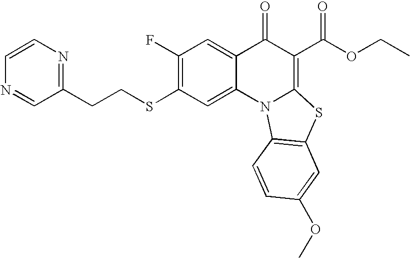

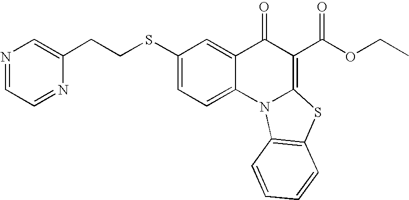

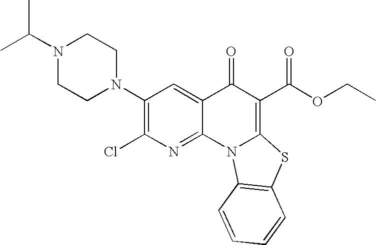

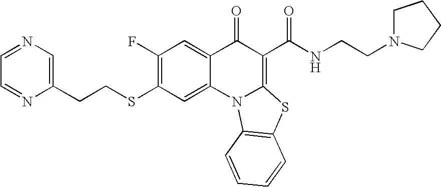

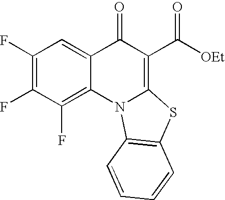

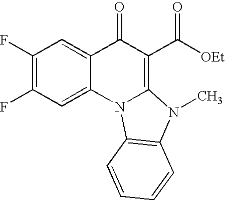

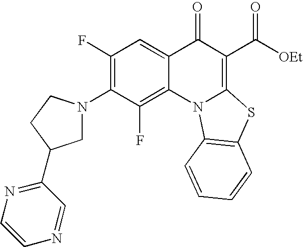

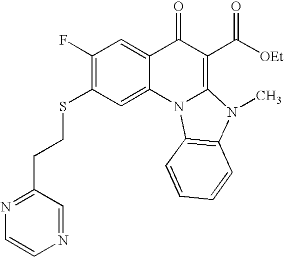

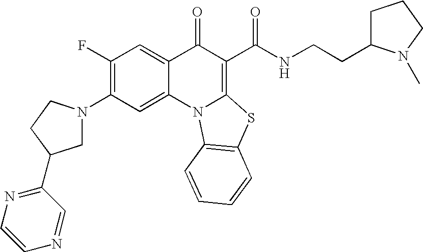

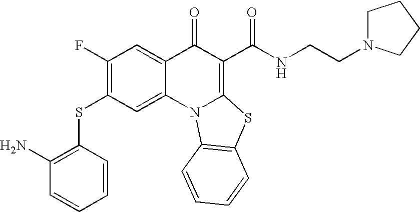

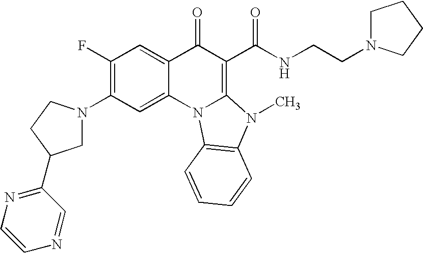

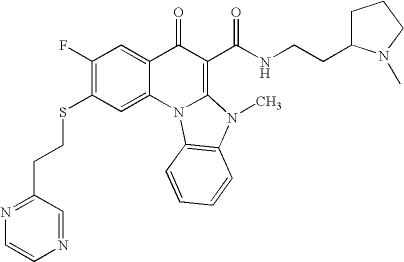

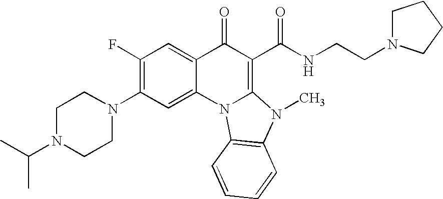

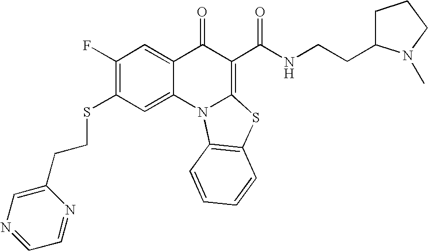

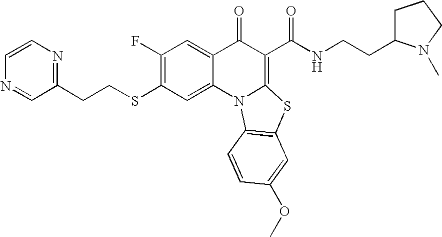

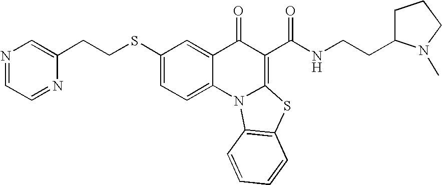

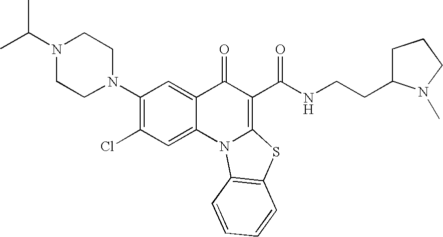

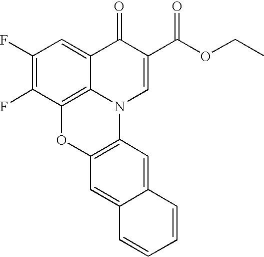

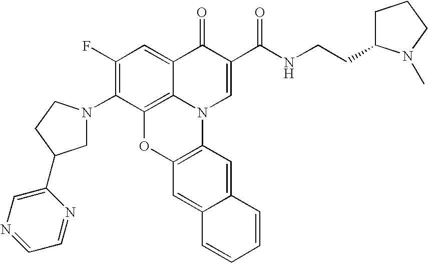

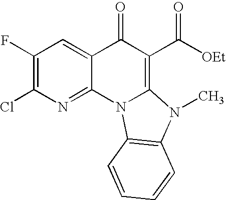

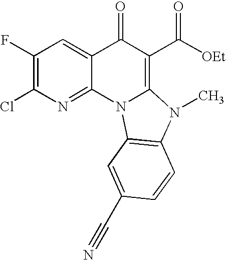

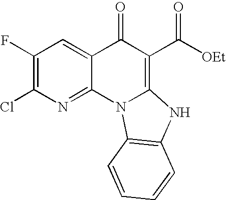

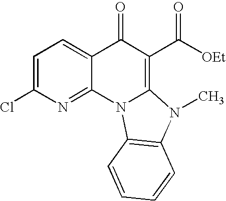

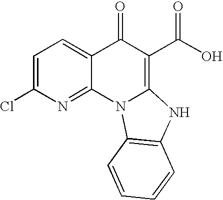

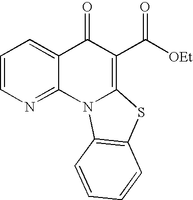

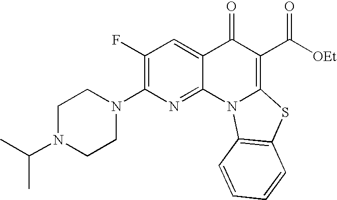

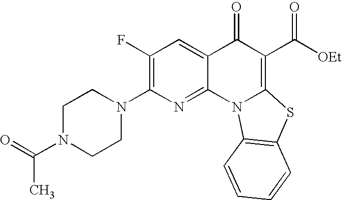

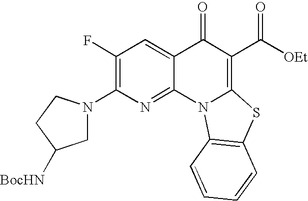

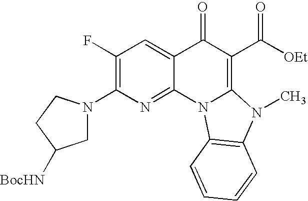

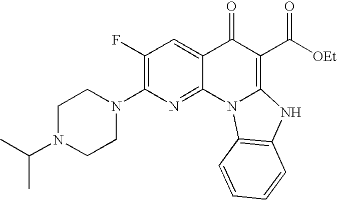

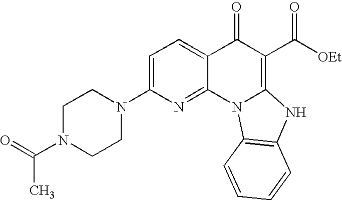

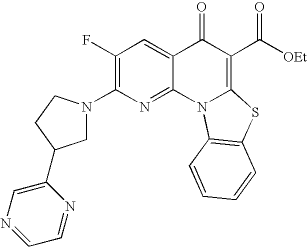

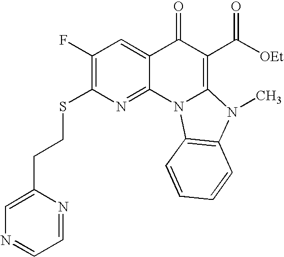

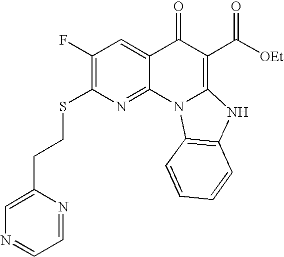

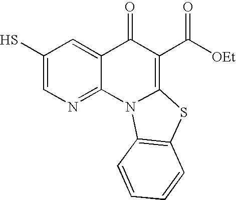

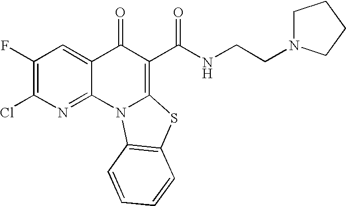

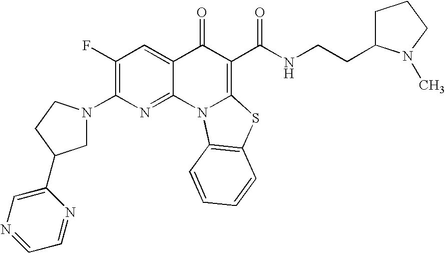

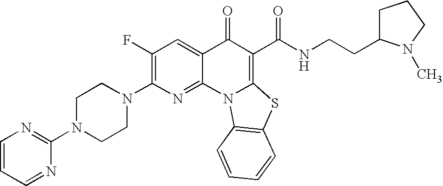

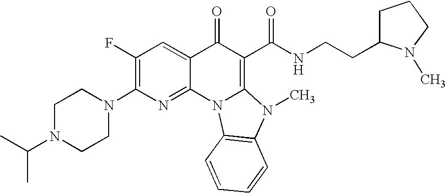

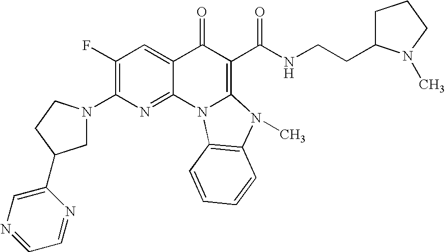

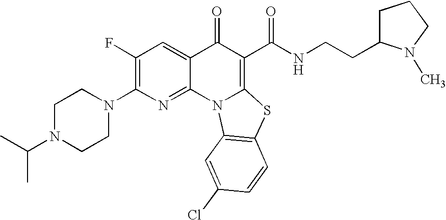

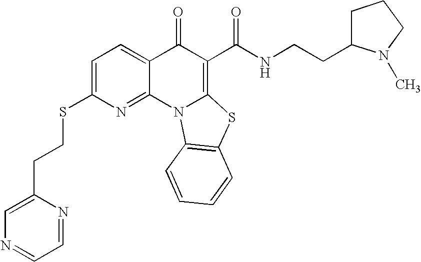

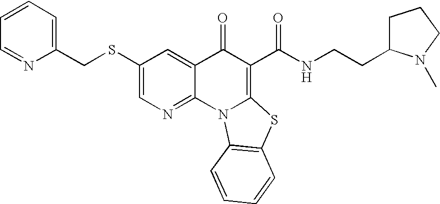

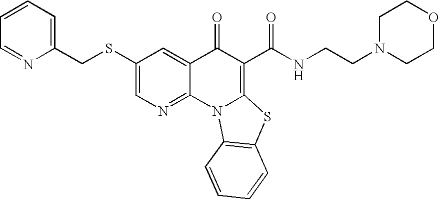

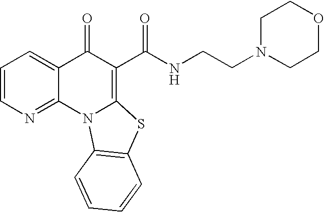

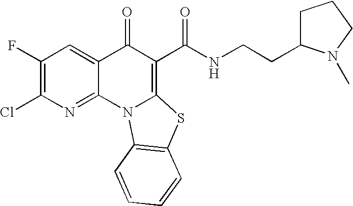

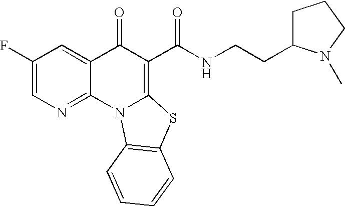

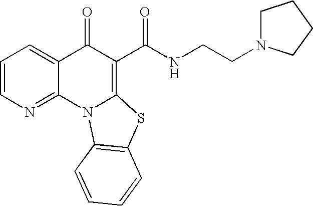

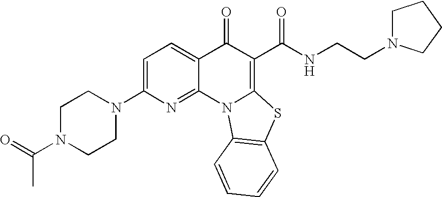

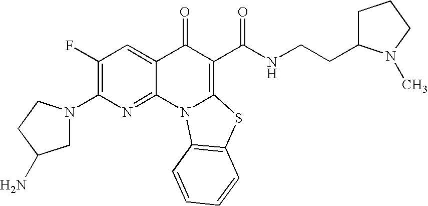

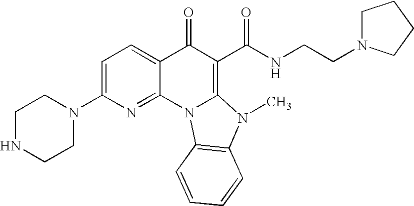

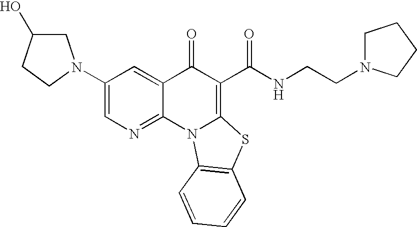

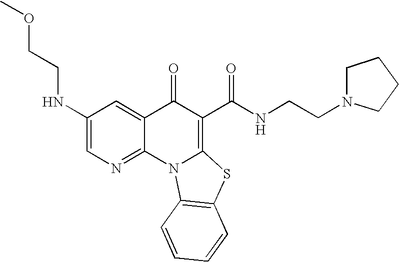

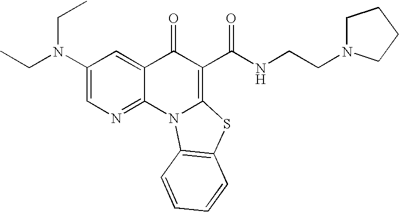

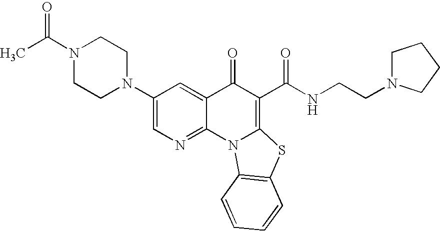

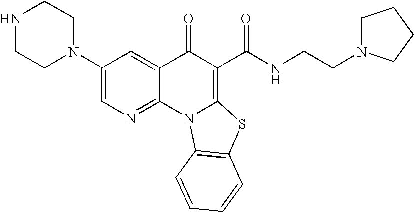

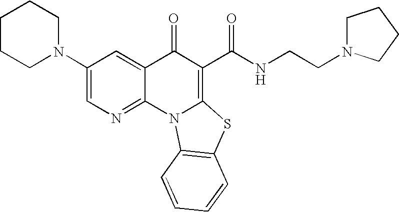

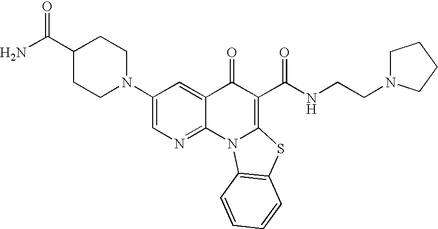

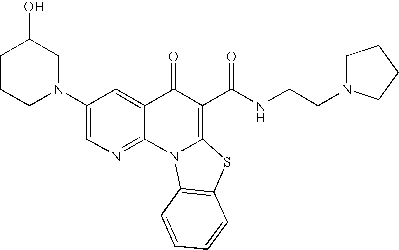

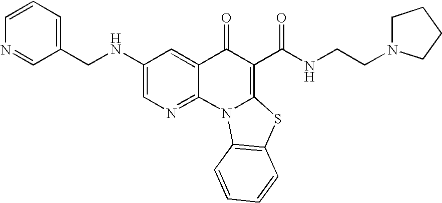

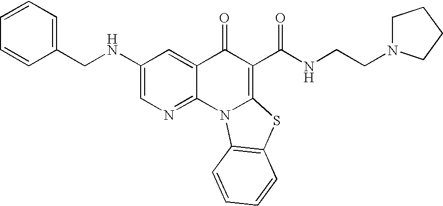

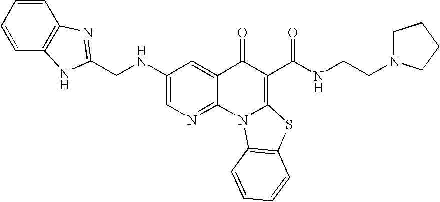

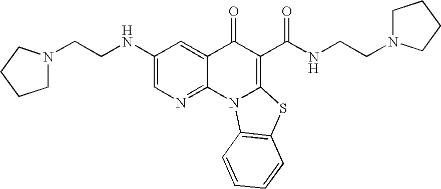

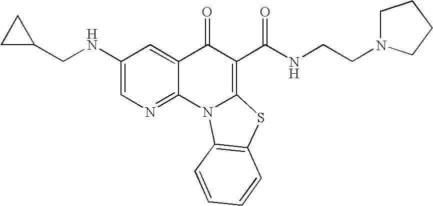

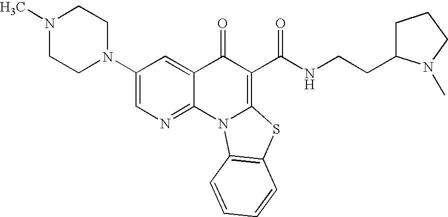

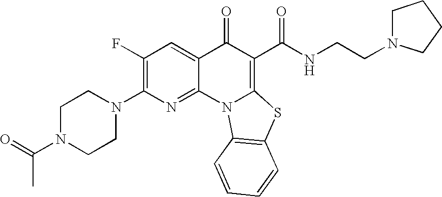

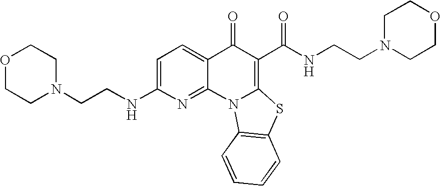

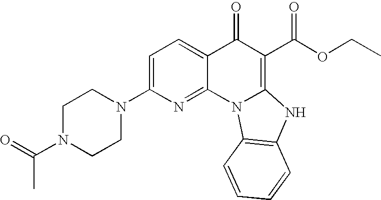

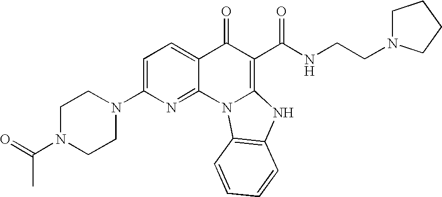

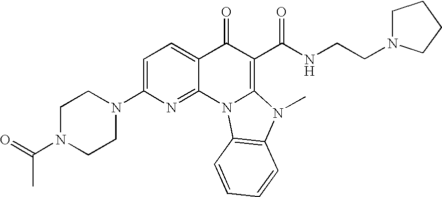

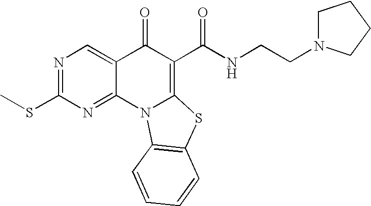

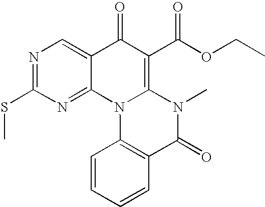

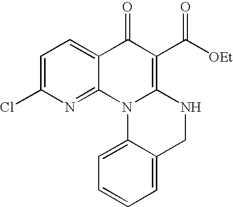

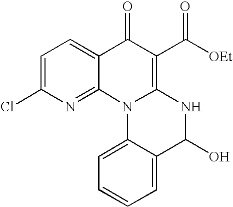

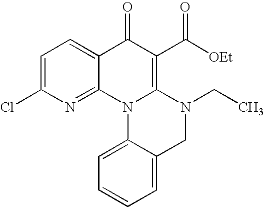

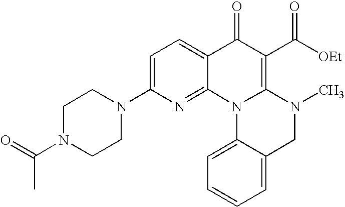

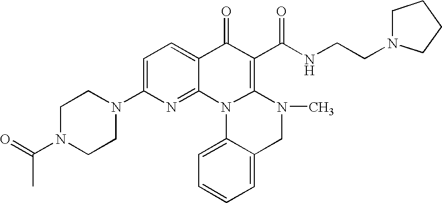

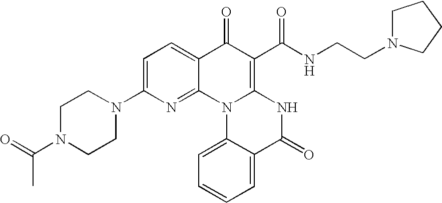

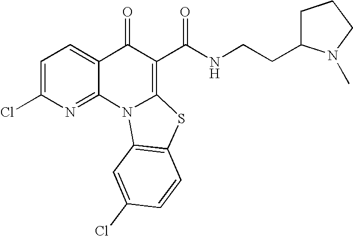

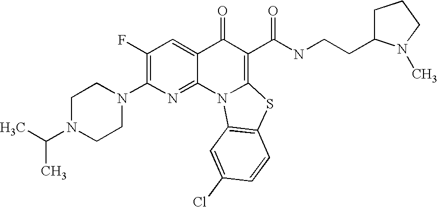

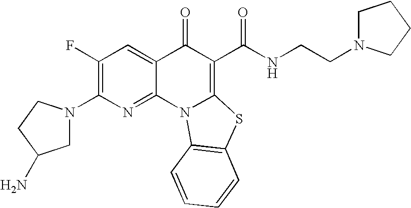

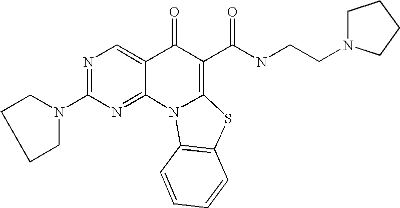

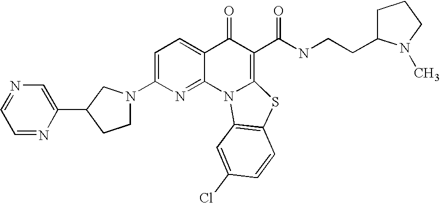

TABLE 3

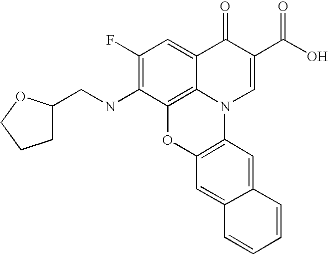

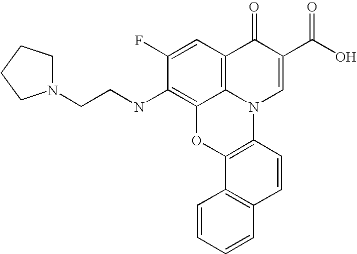

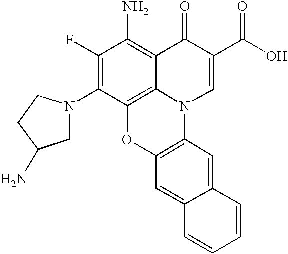

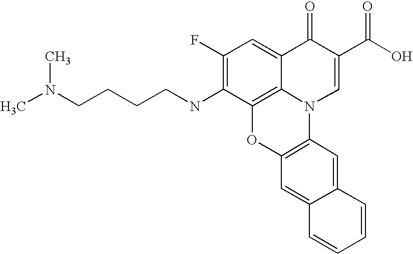

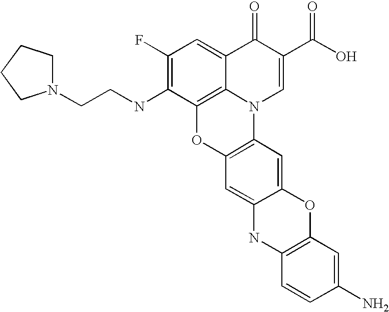

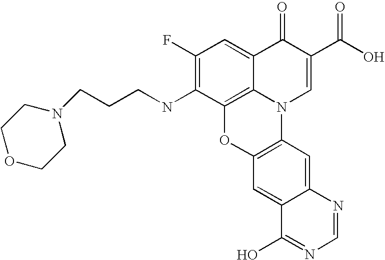

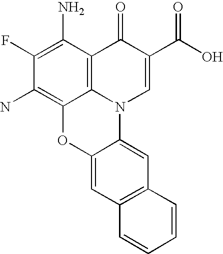

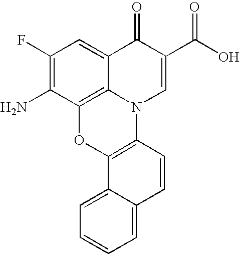

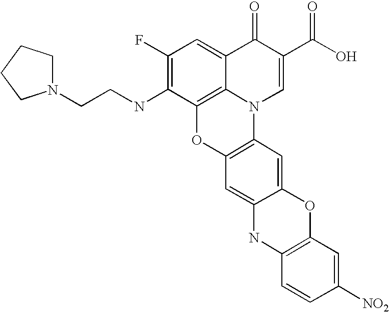

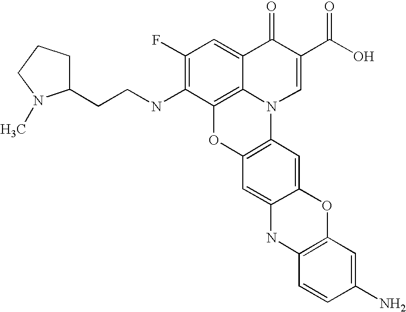

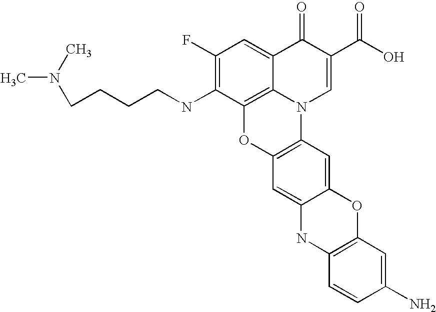

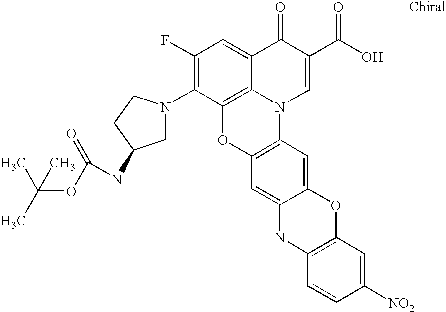

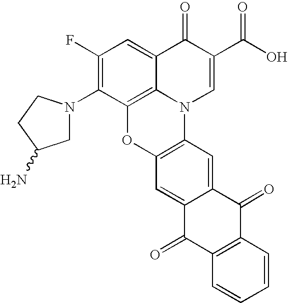

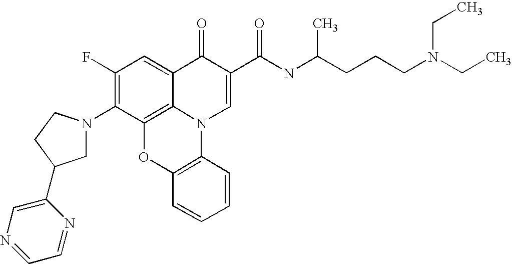

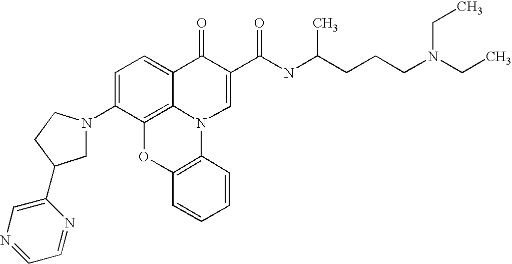

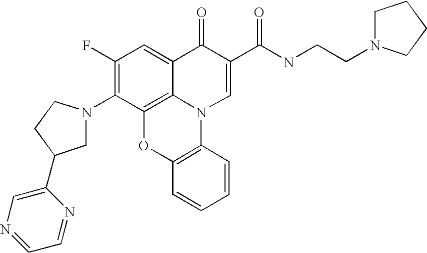

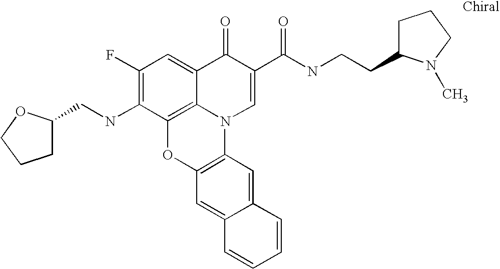

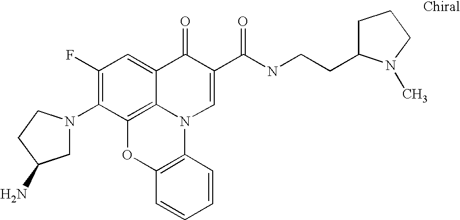

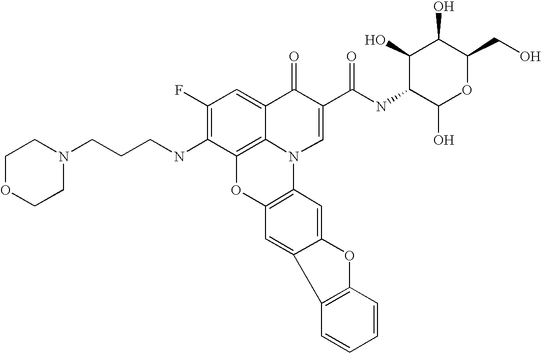

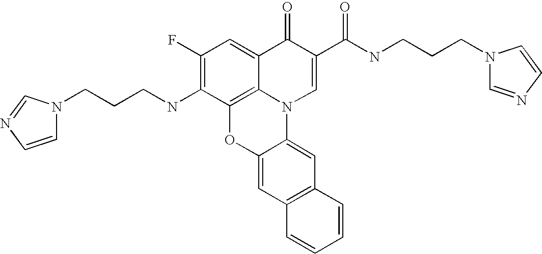

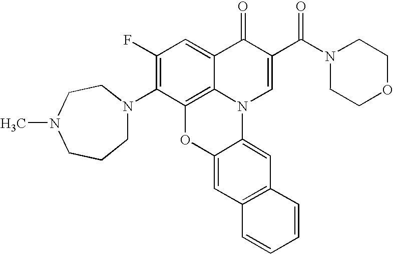

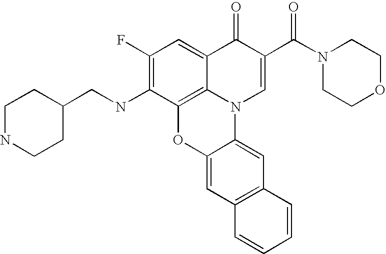

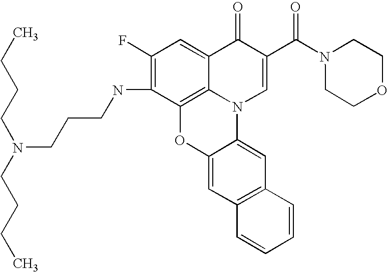

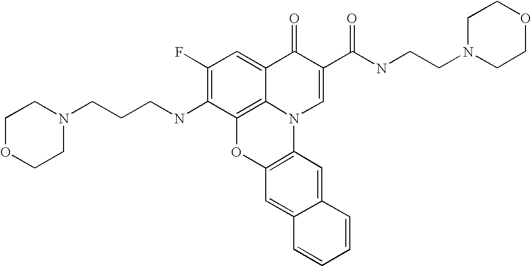

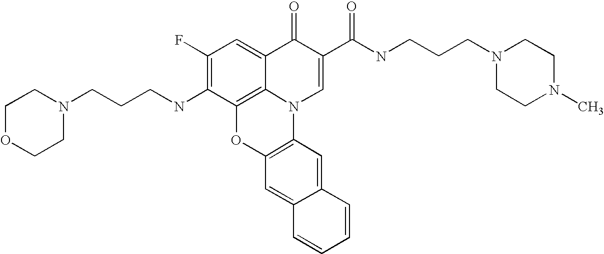

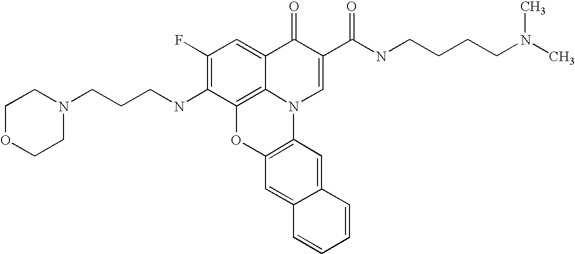

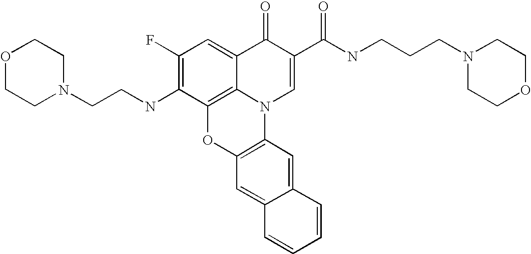

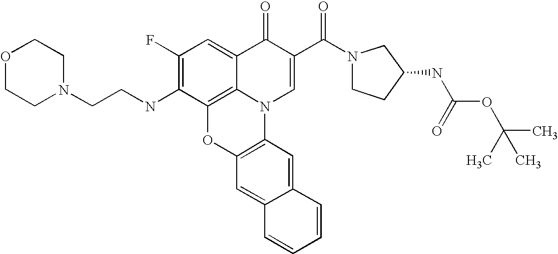

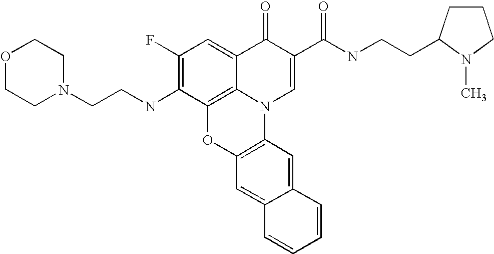

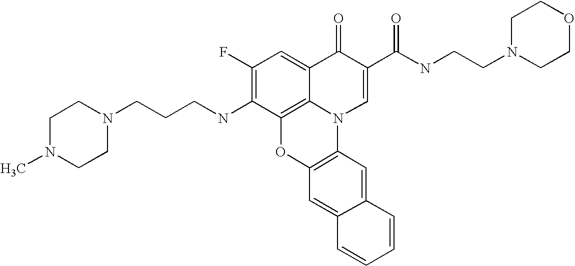

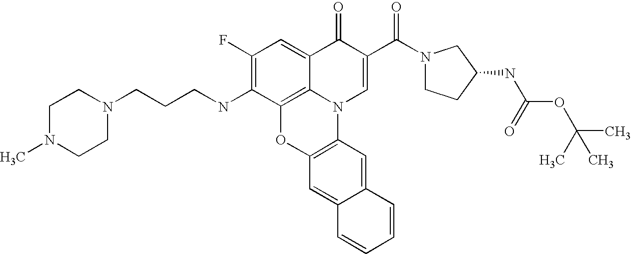

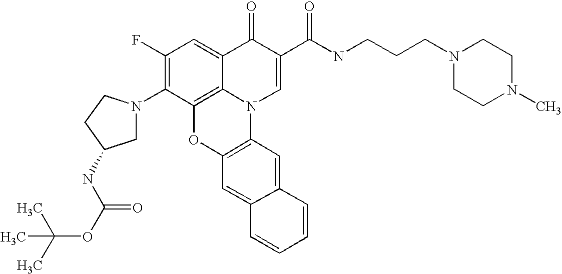

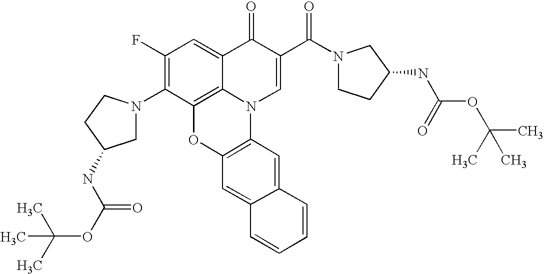

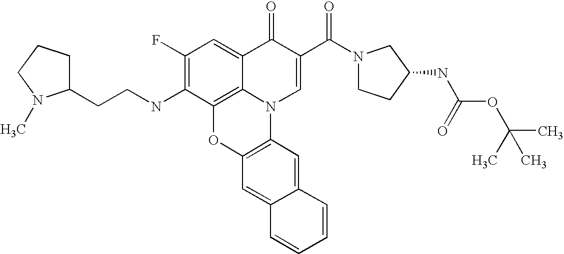

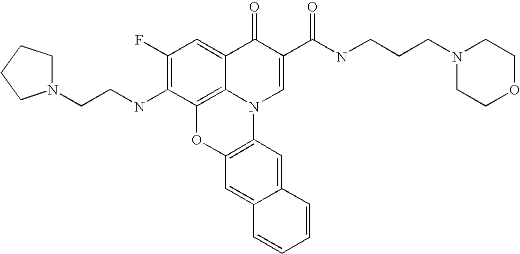

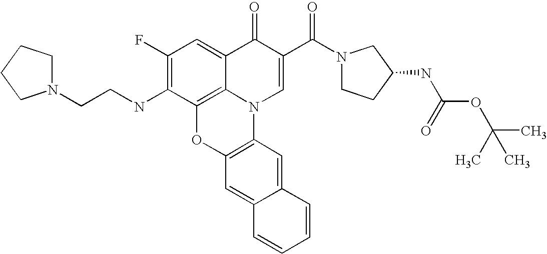

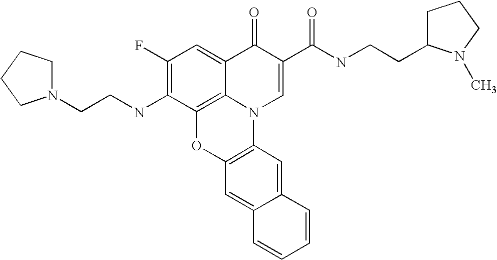

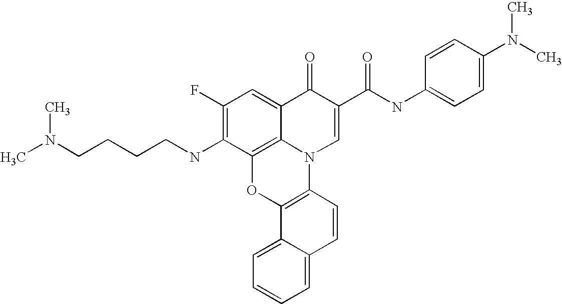

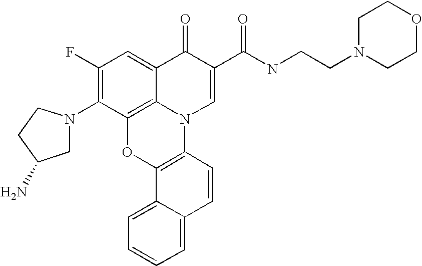

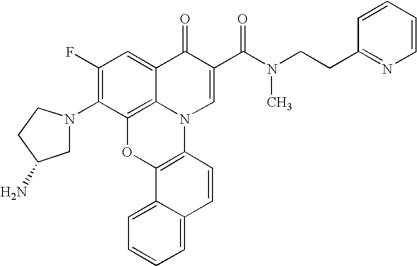

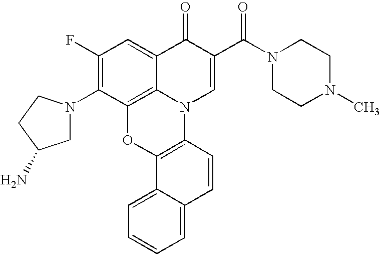

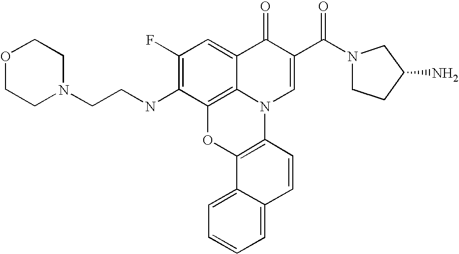

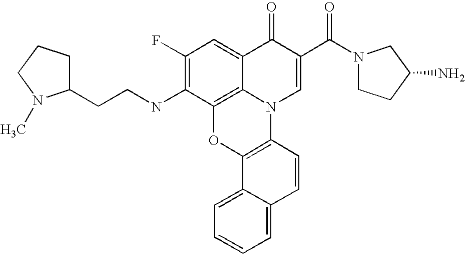

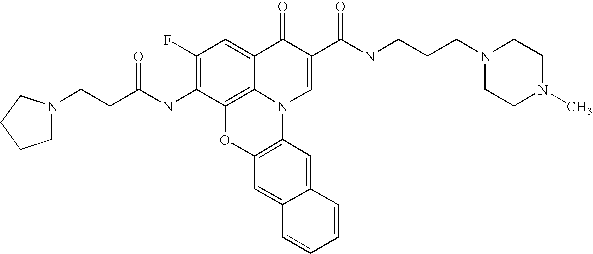

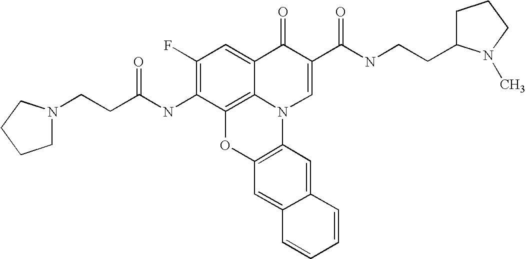

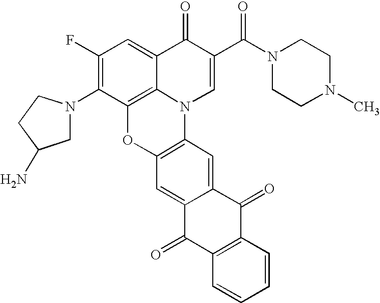

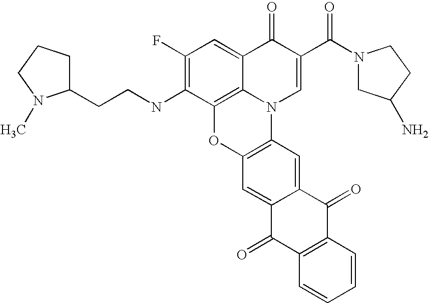

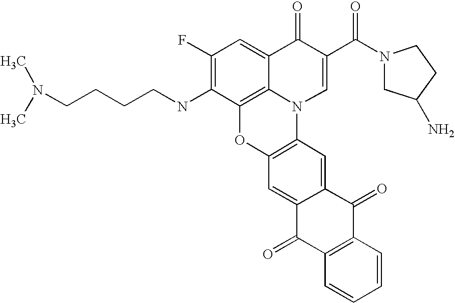

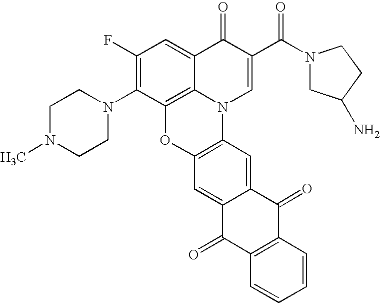

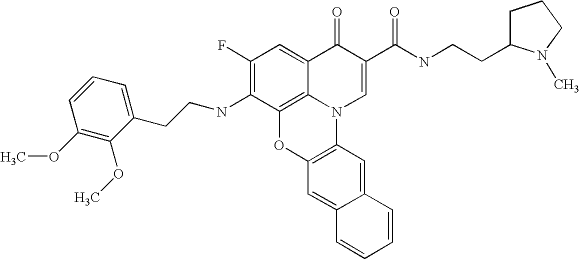

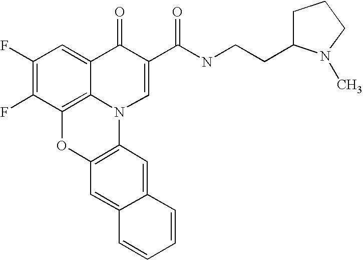

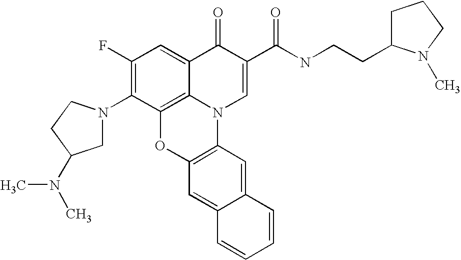

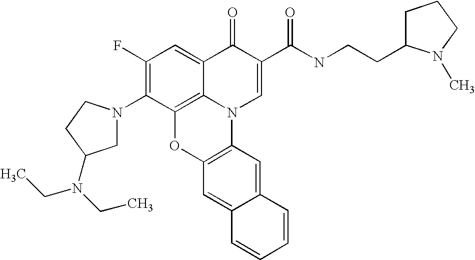

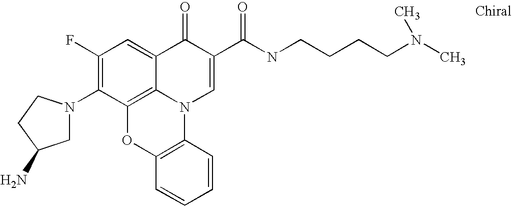

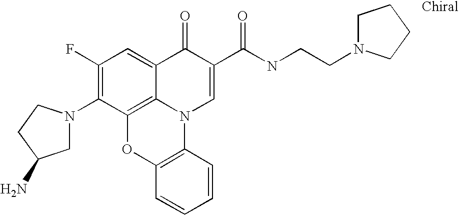

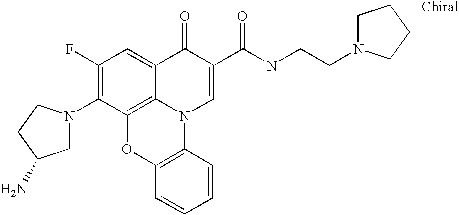

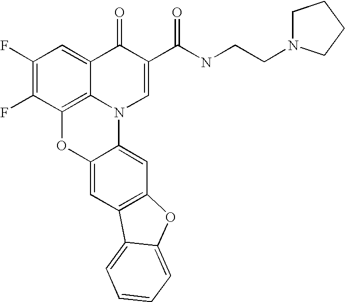

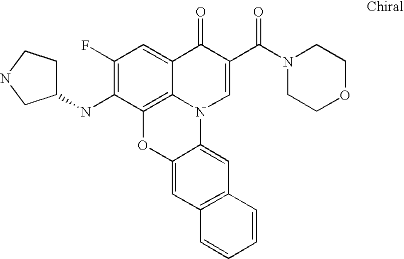

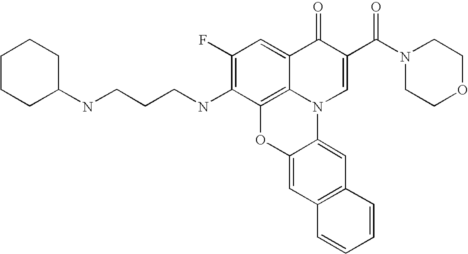

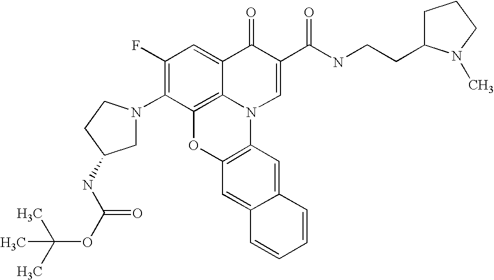

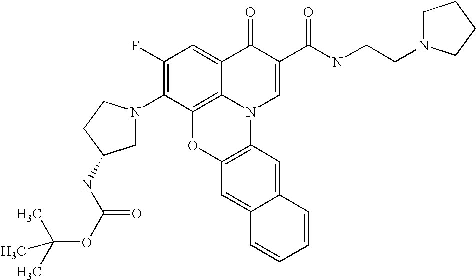

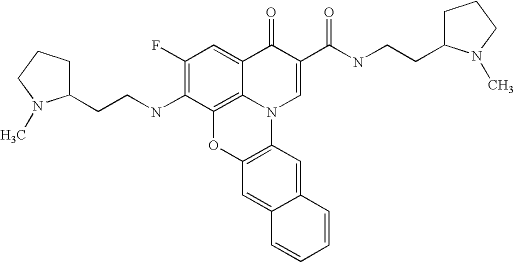

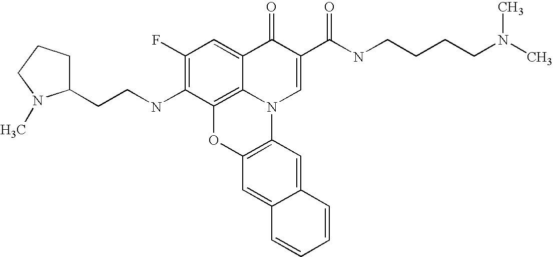

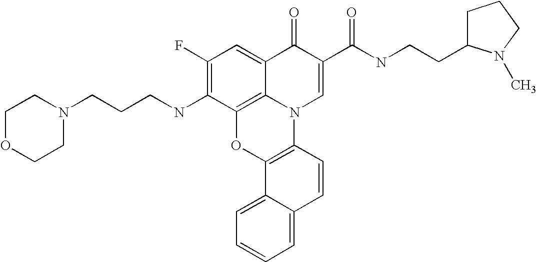

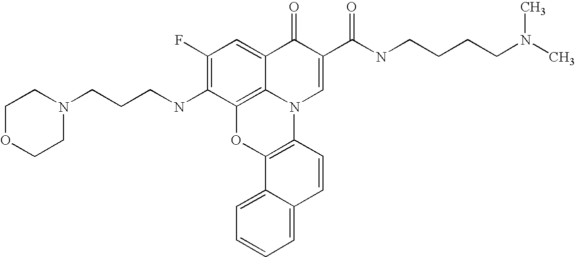

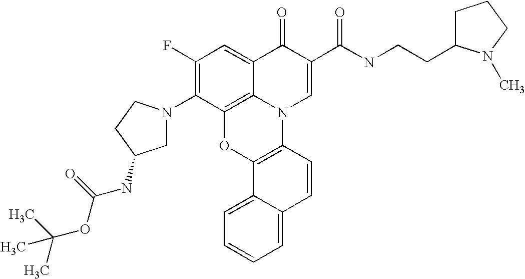

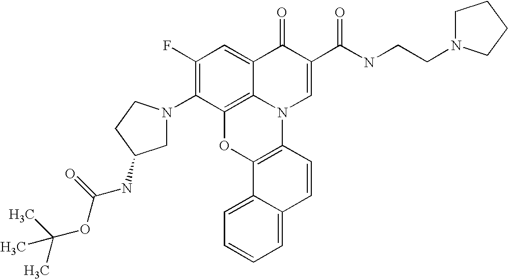

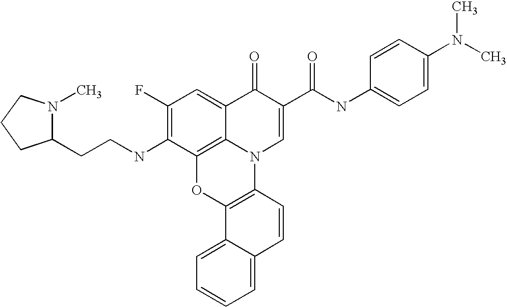

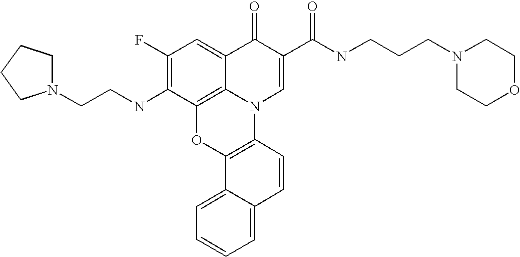

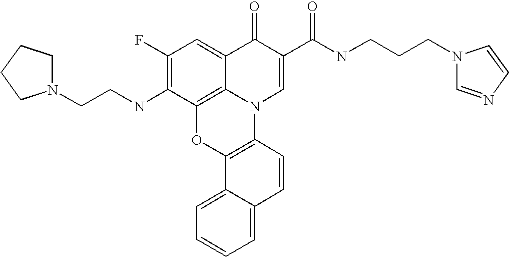

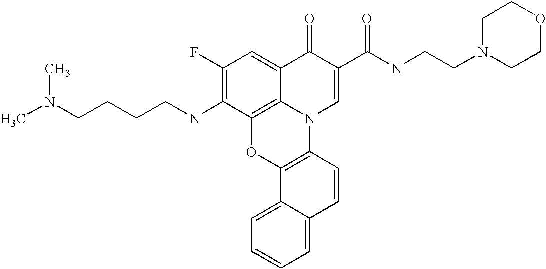

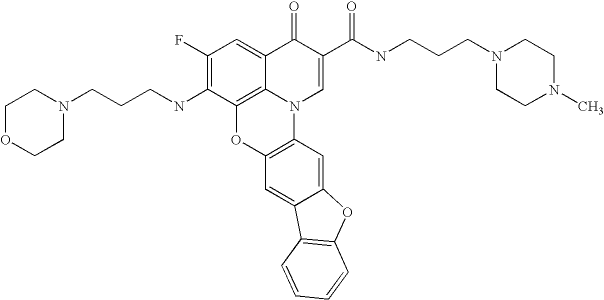

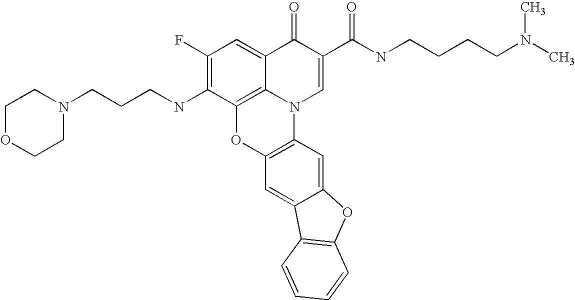

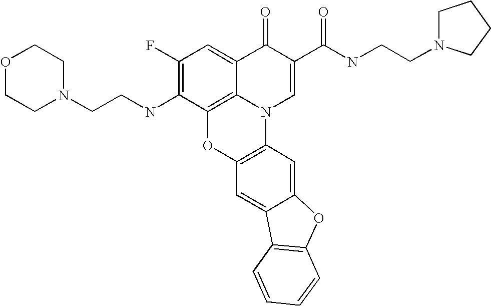

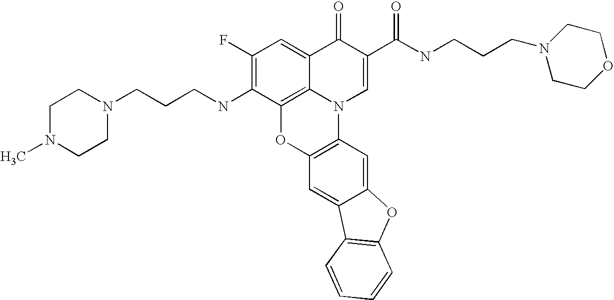

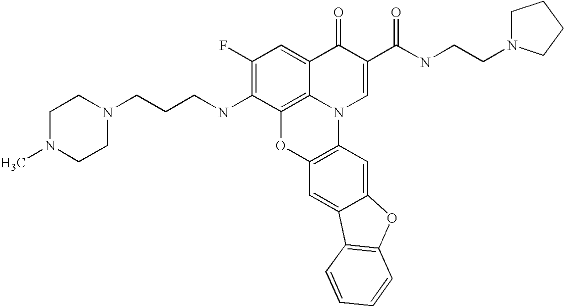

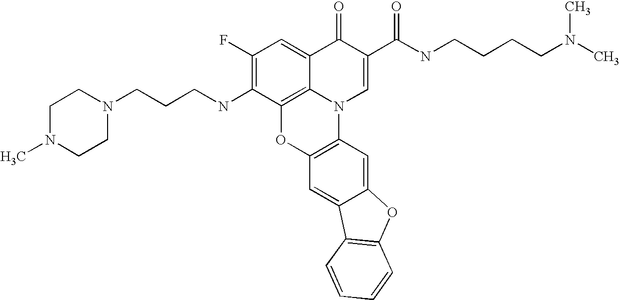

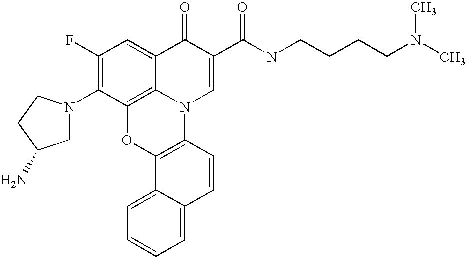

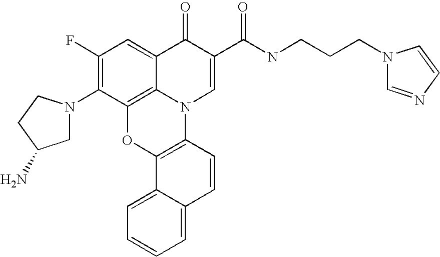

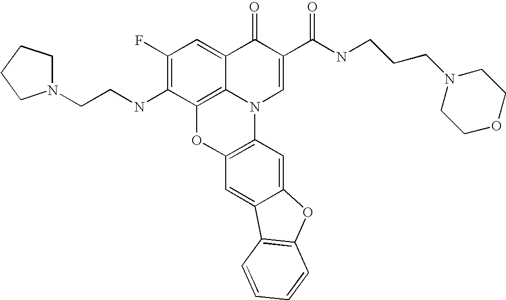

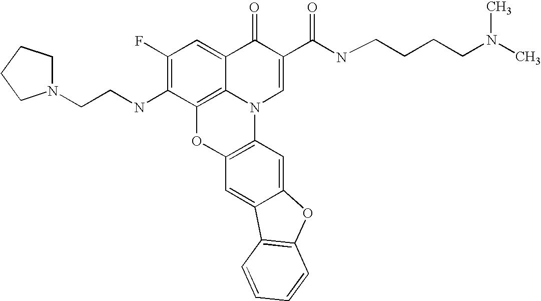

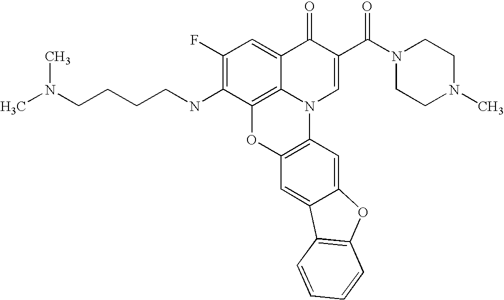

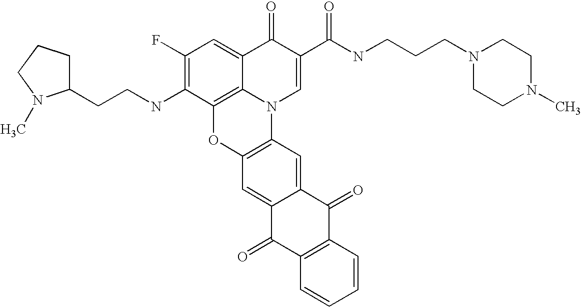

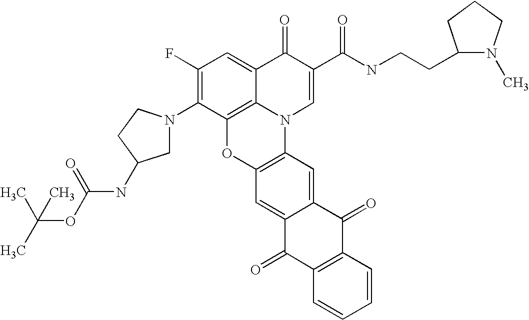

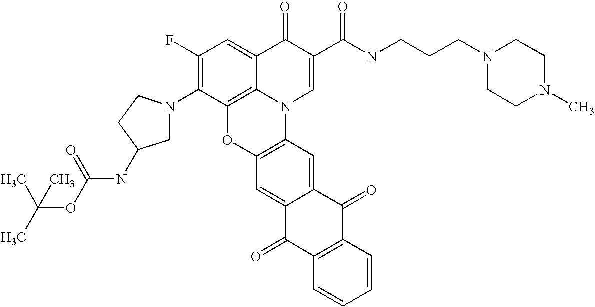

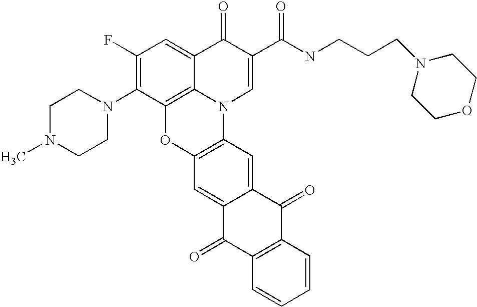

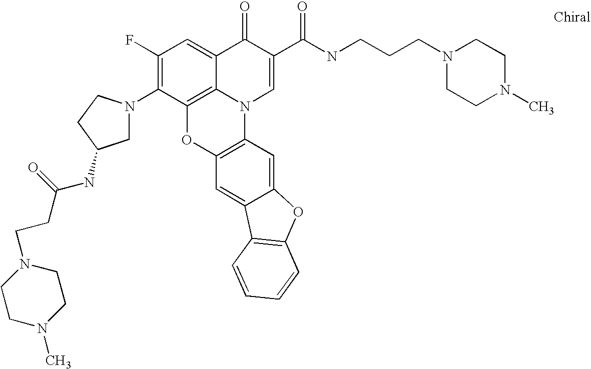

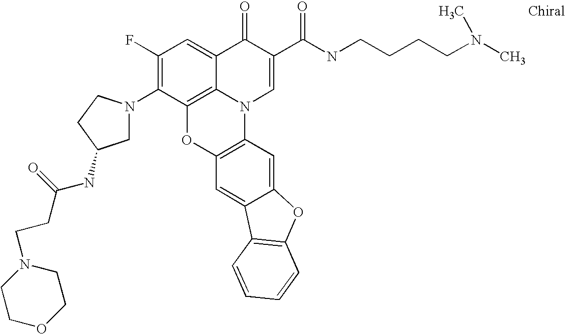

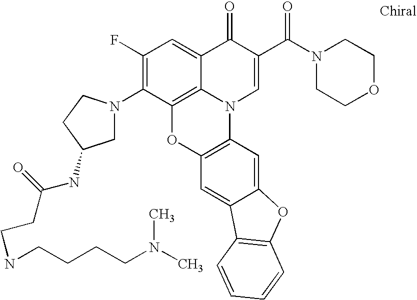

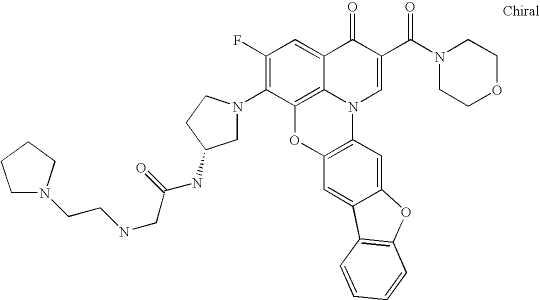

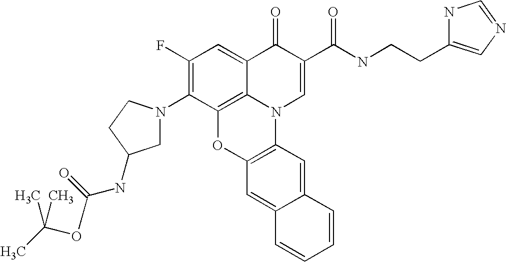

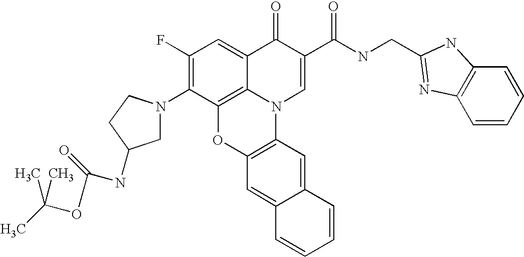

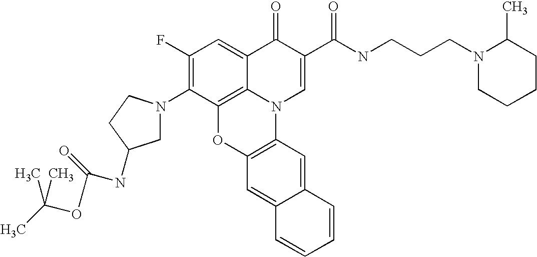

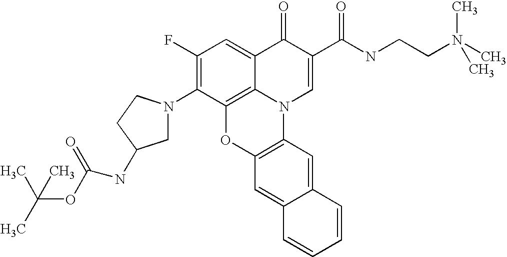

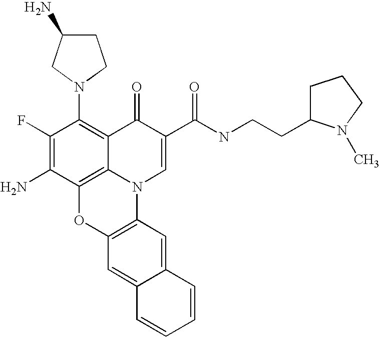

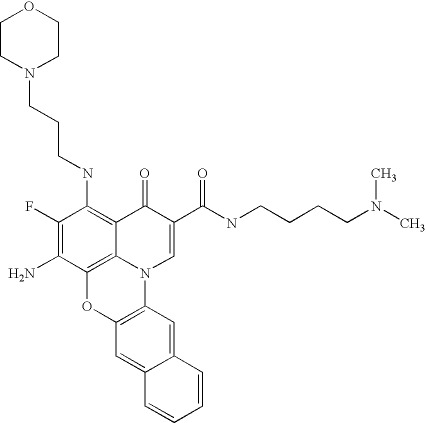

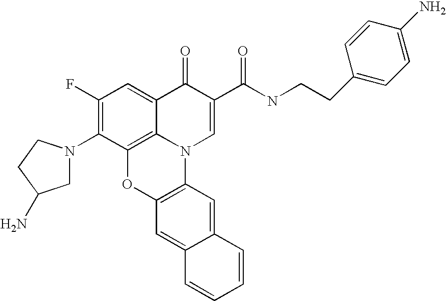

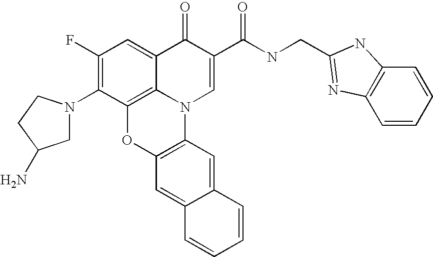

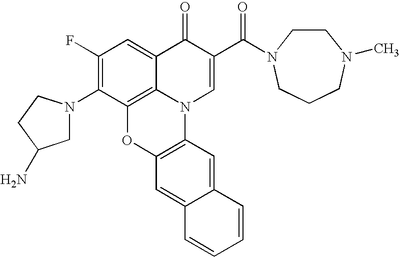

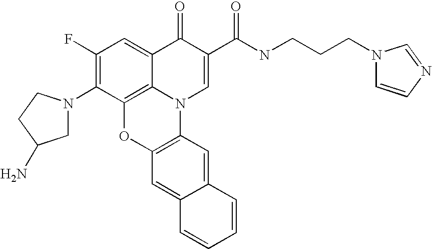

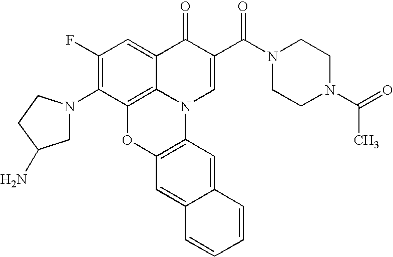

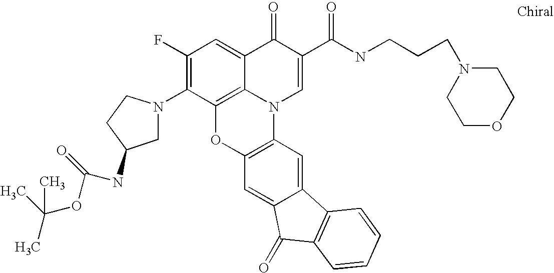

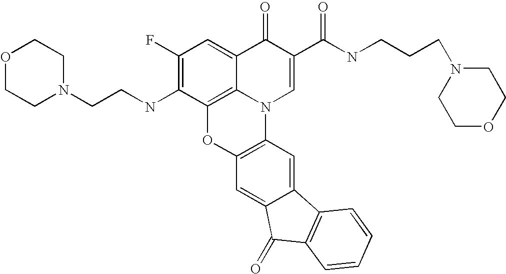

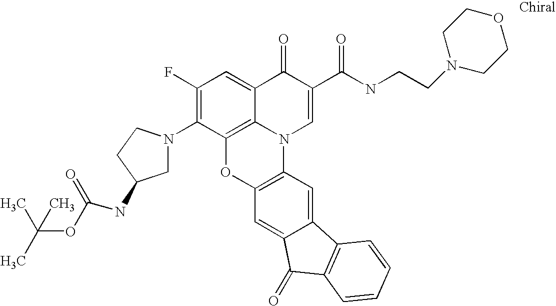

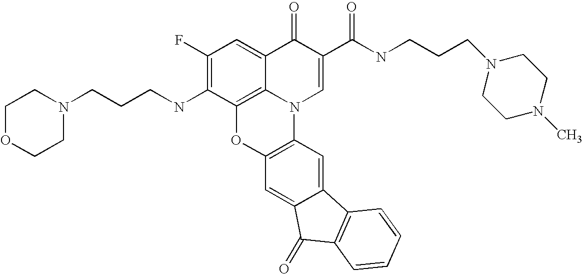

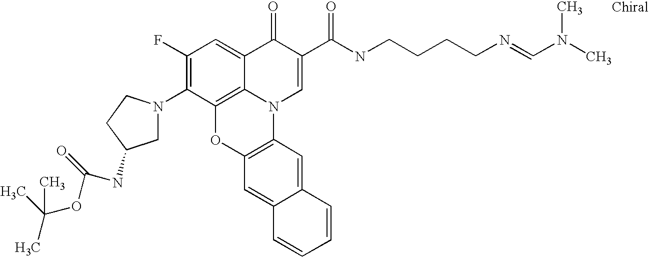

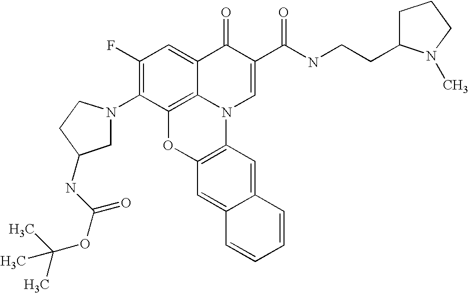

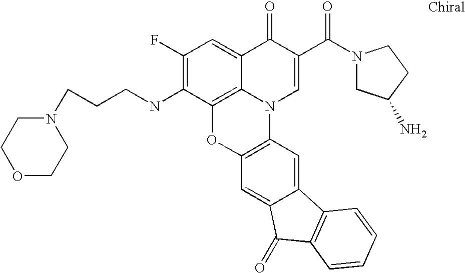

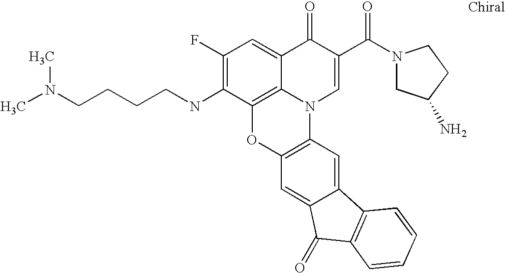

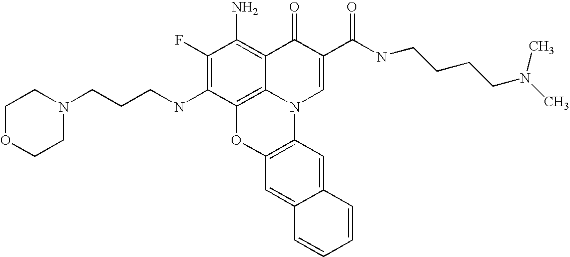

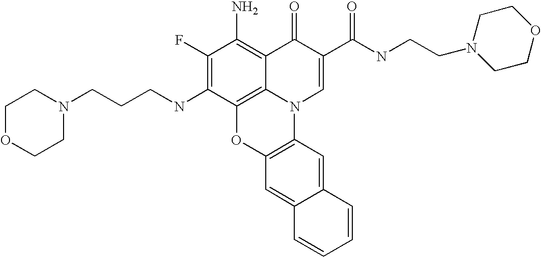

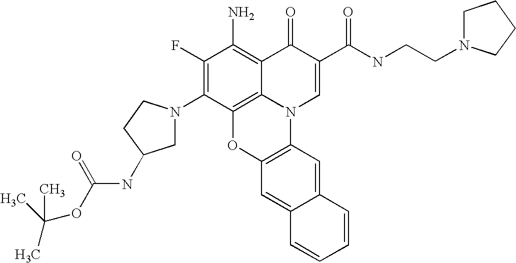

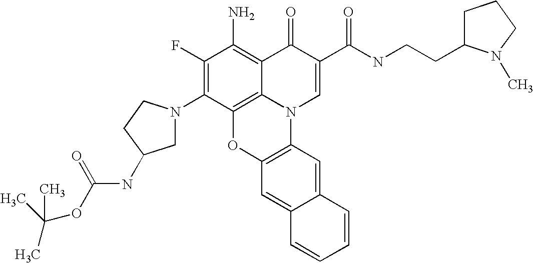

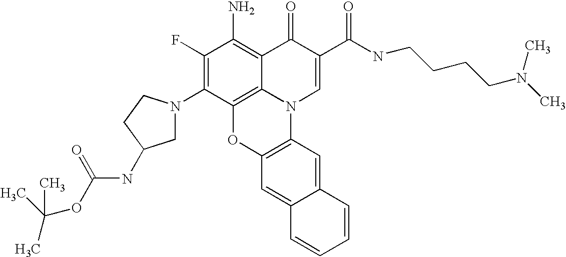

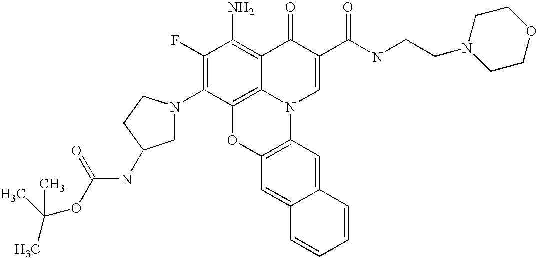

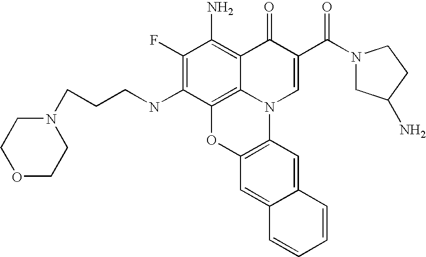

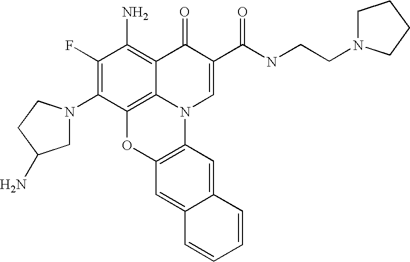

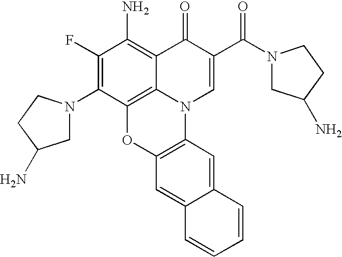

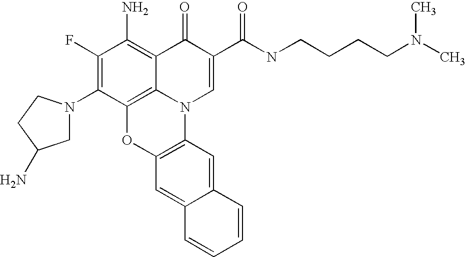

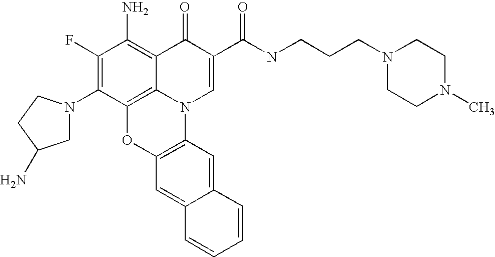

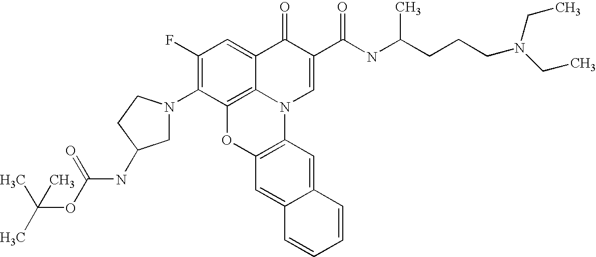

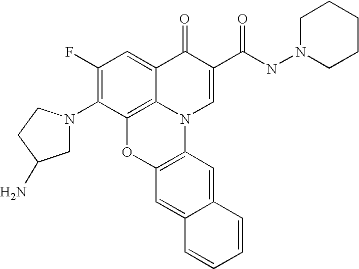

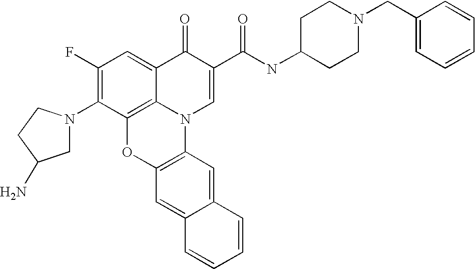

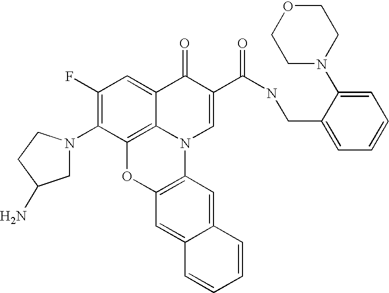

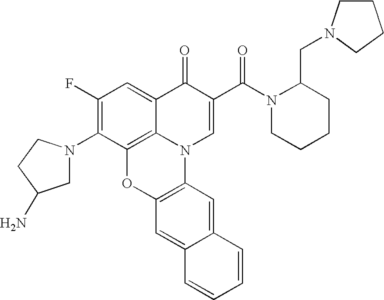

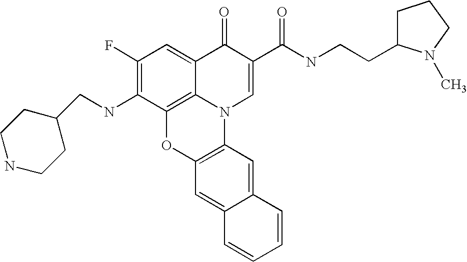

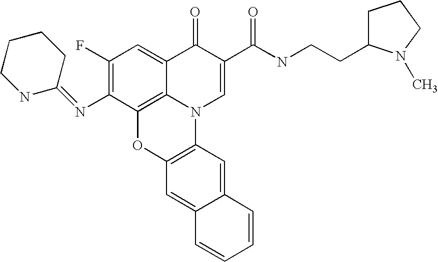

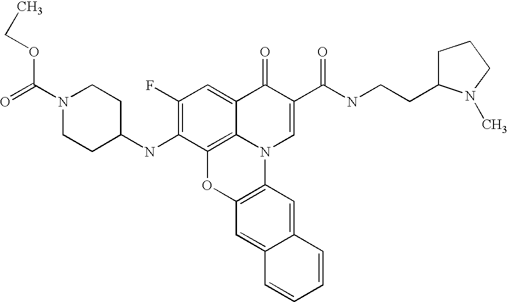

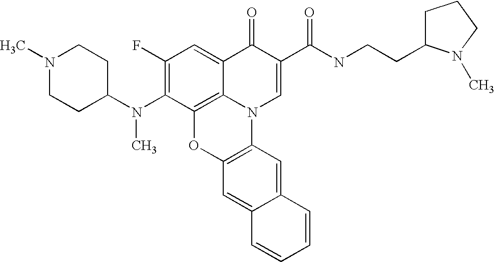

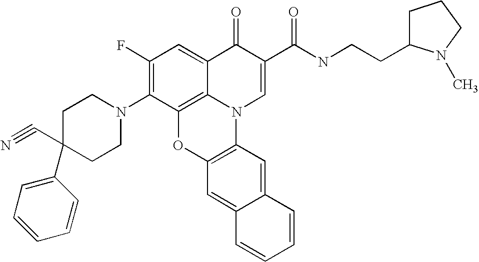

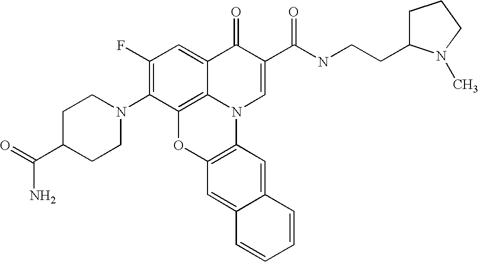

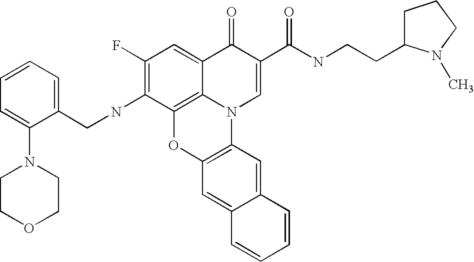

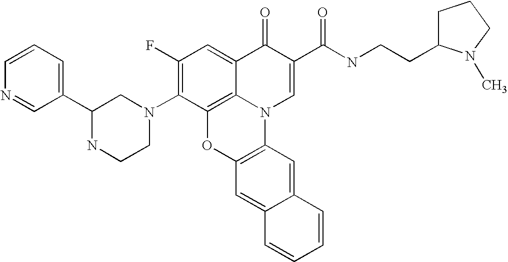

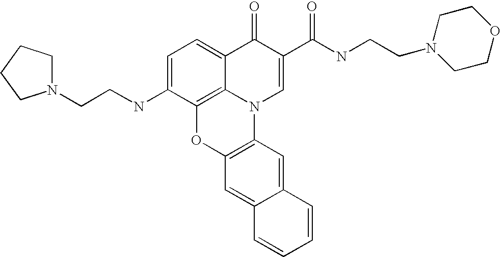

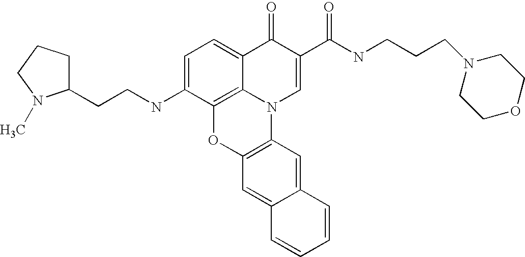

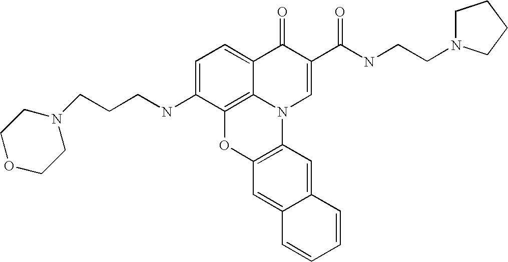

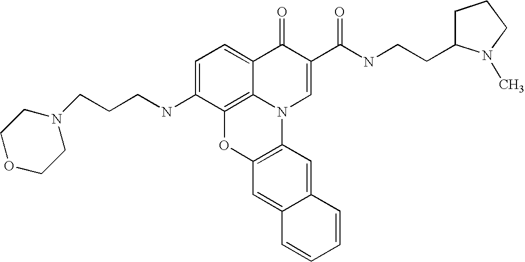

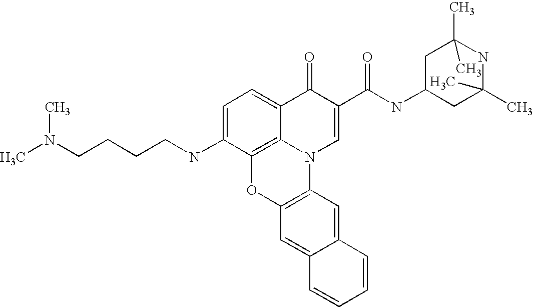

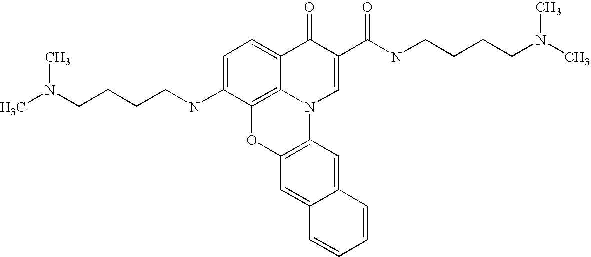

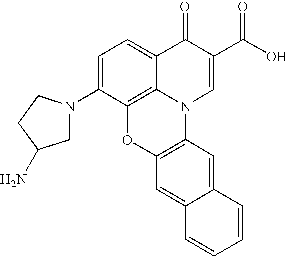

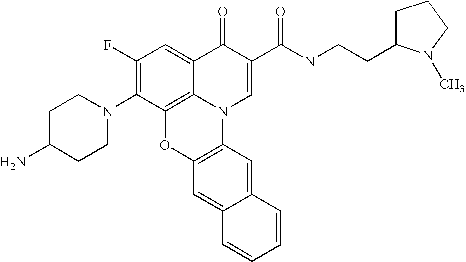

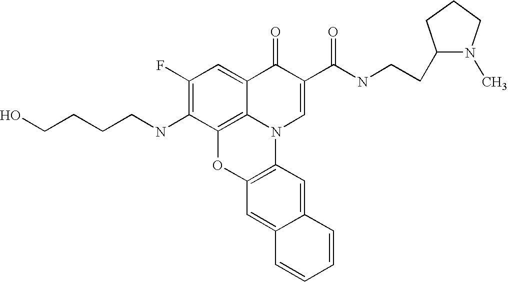

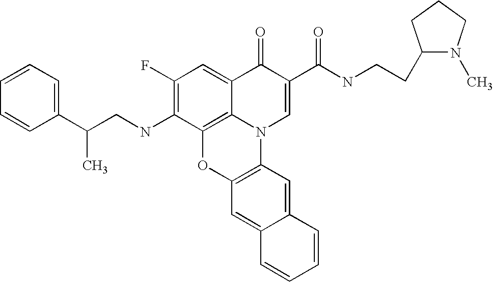

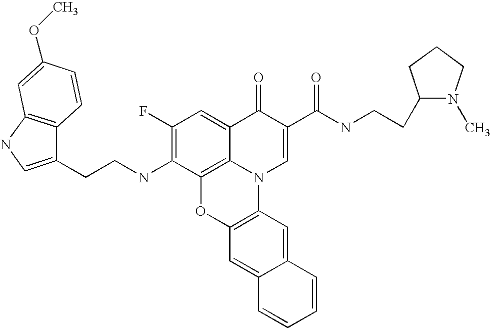

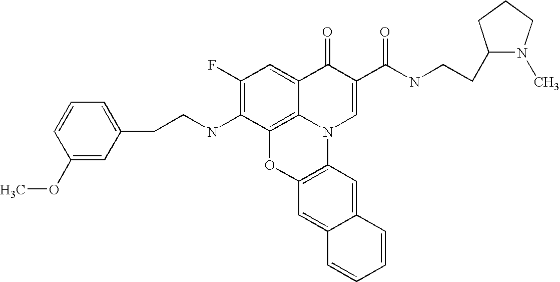

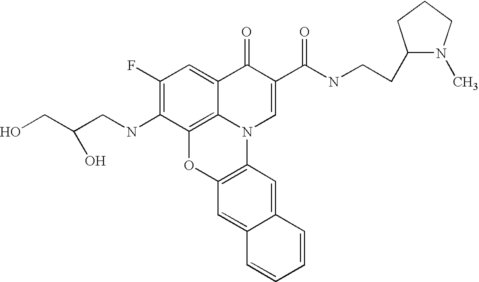

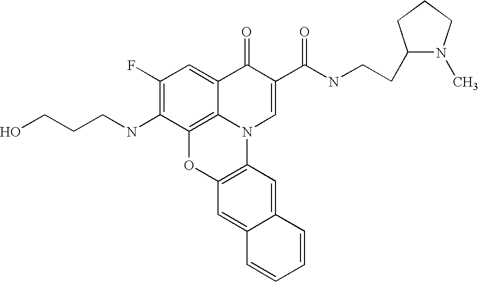

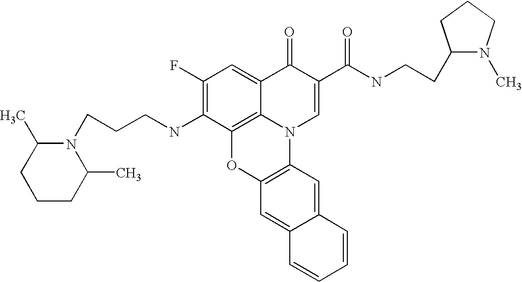

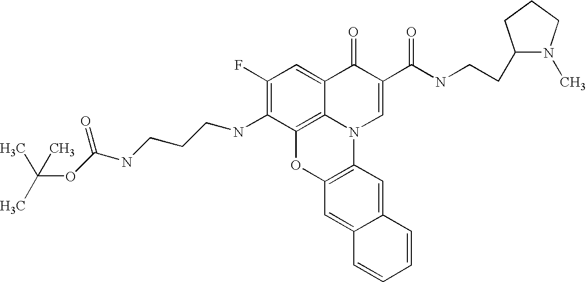

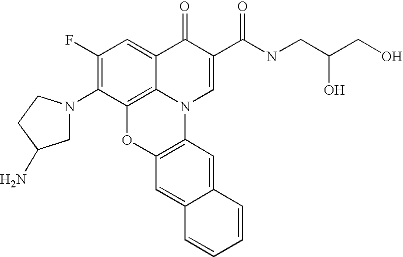

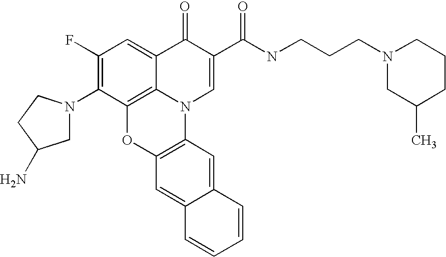

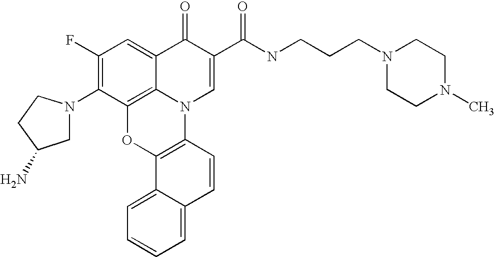

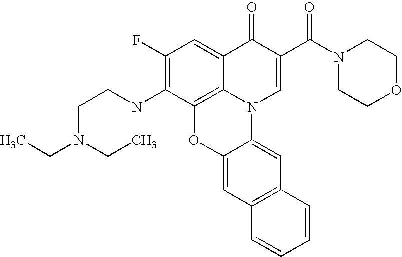

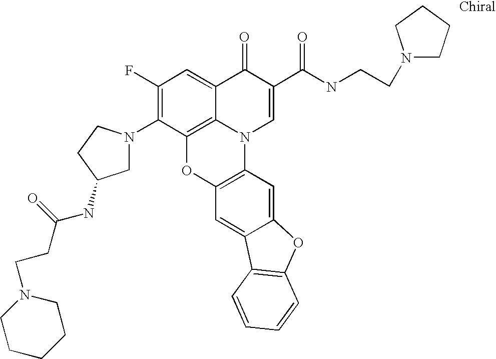

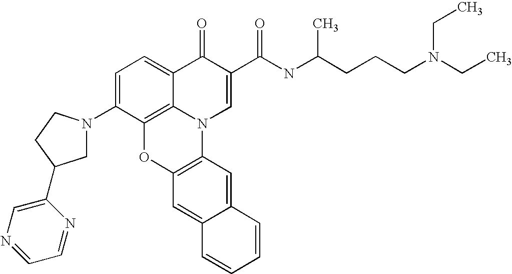

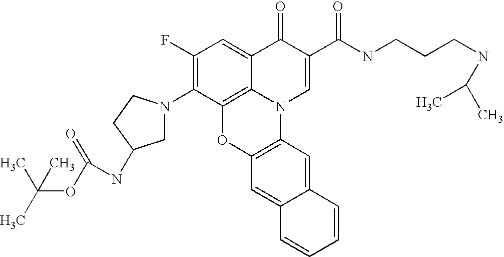

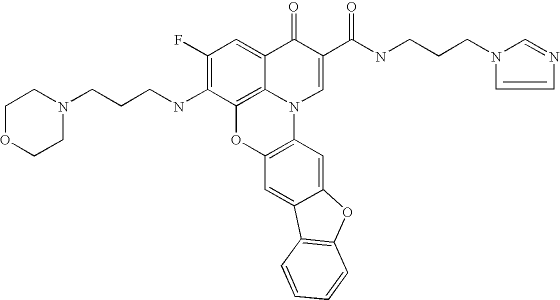

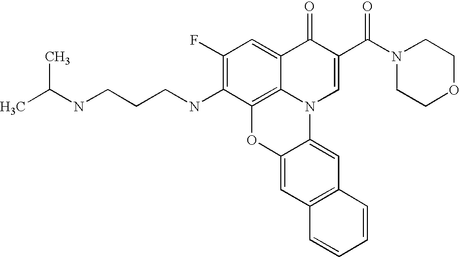

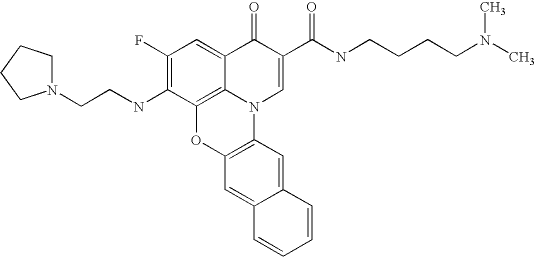

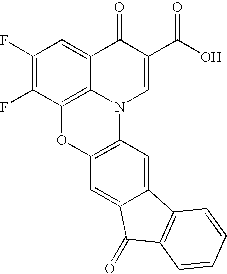

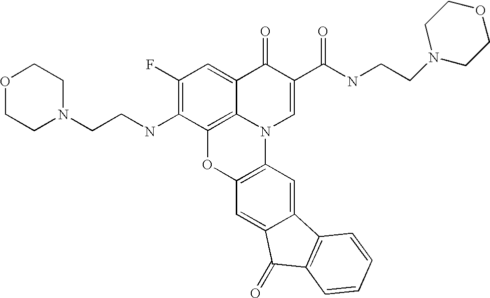

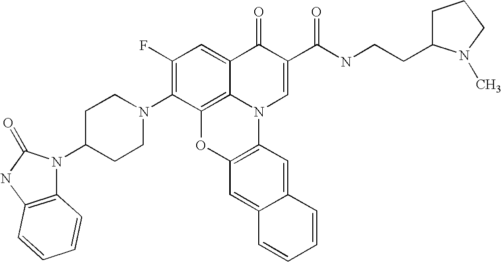

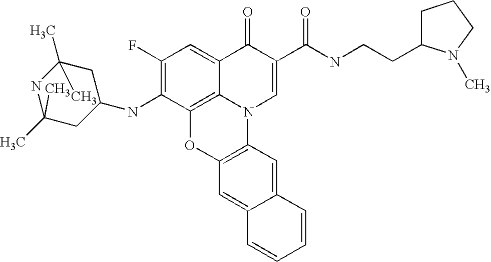

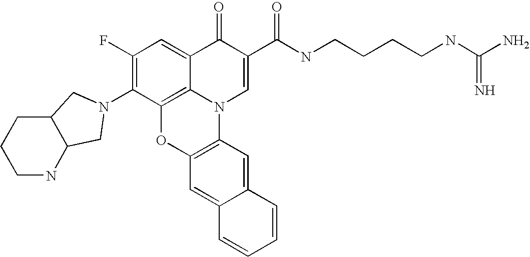

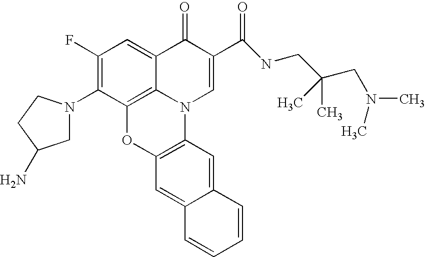

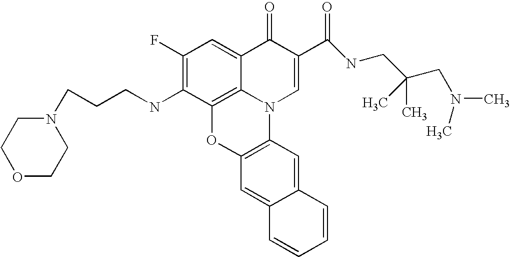

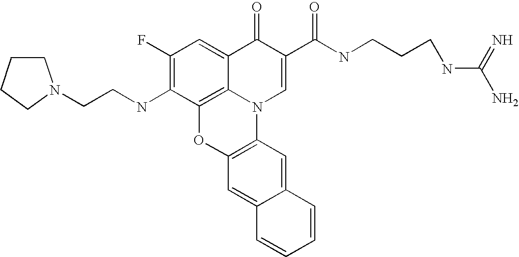

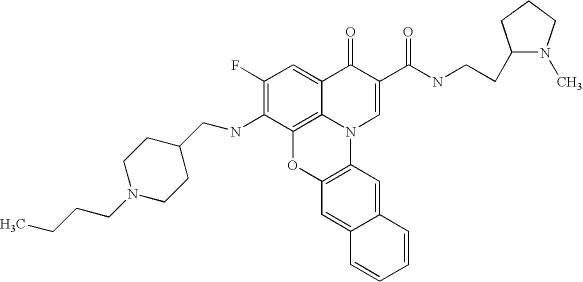

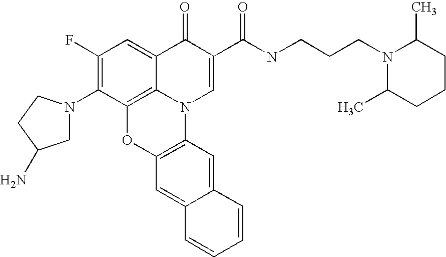

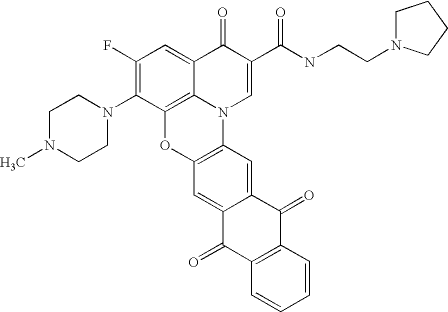

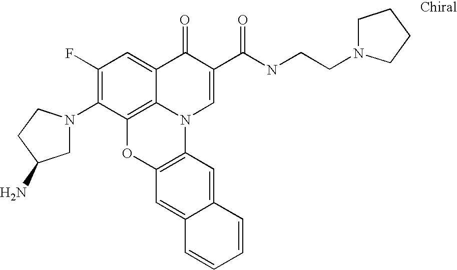

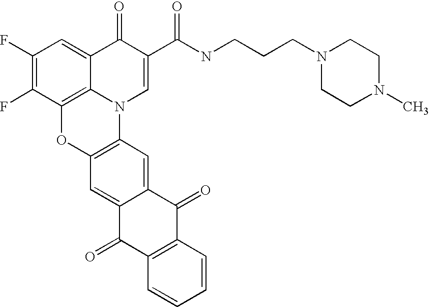

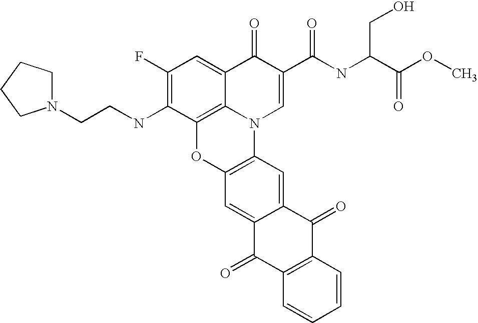

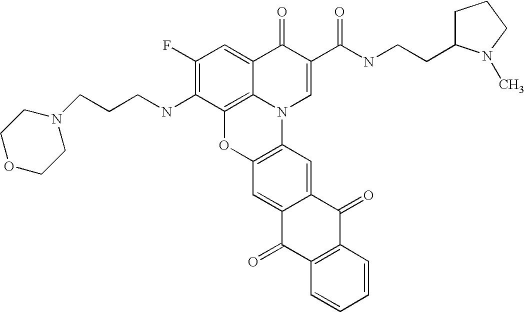

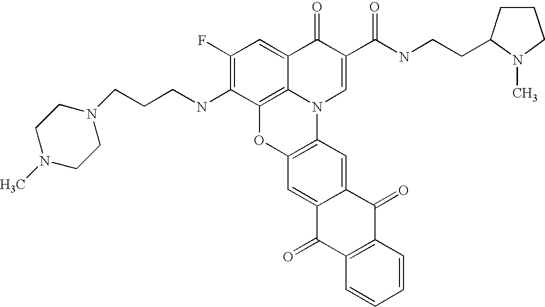

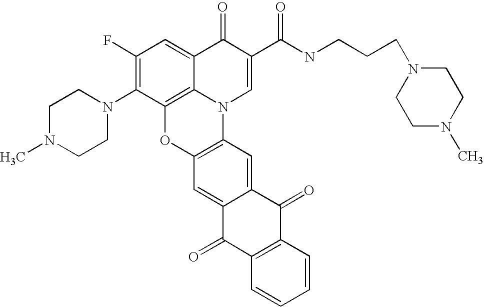

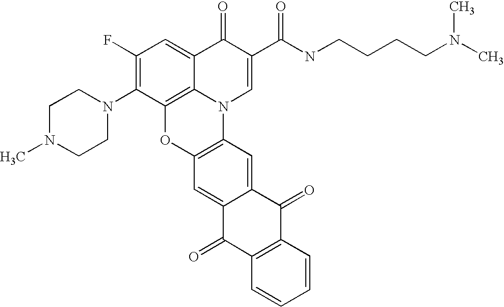

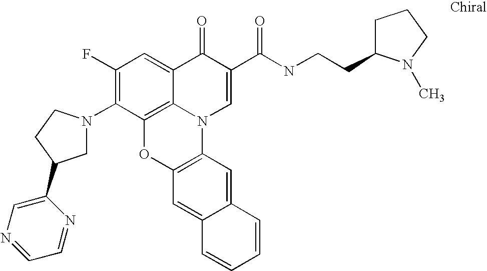

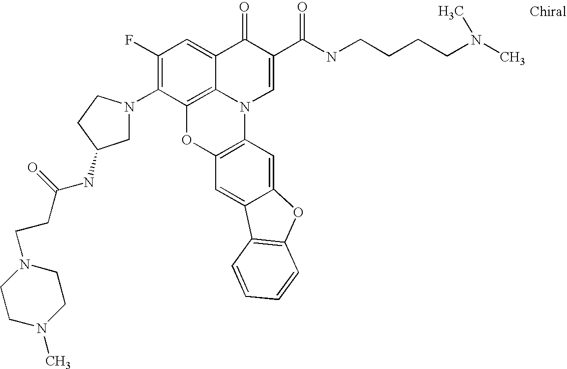

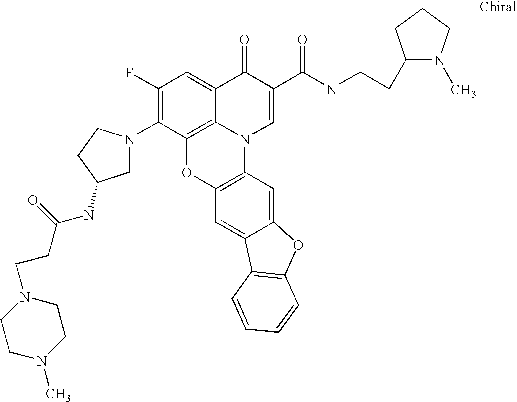

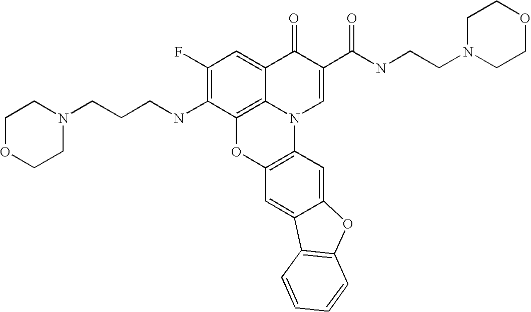

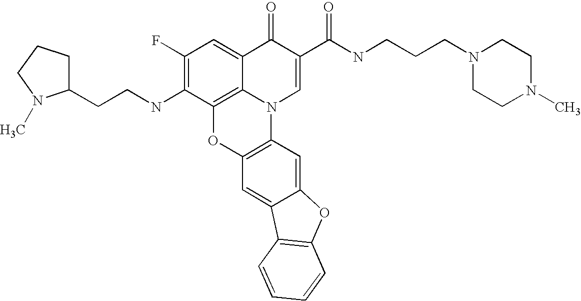

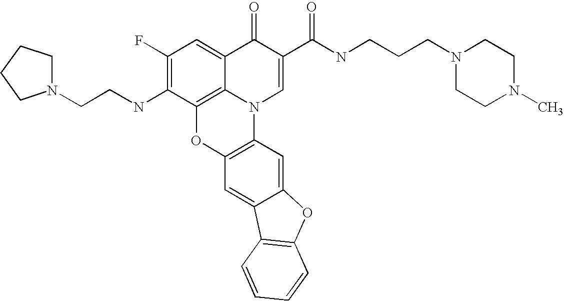

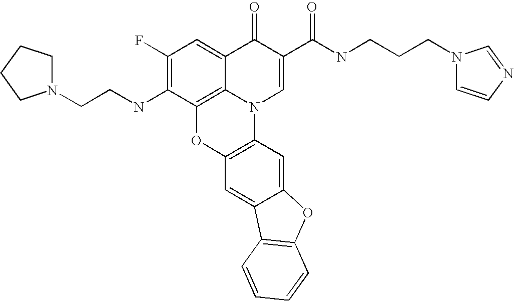

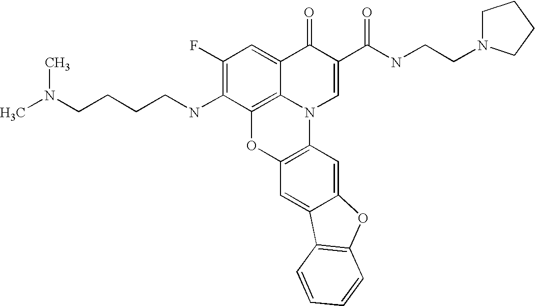

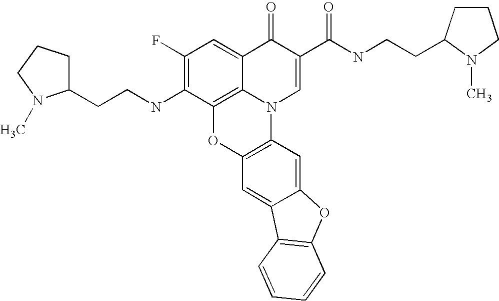

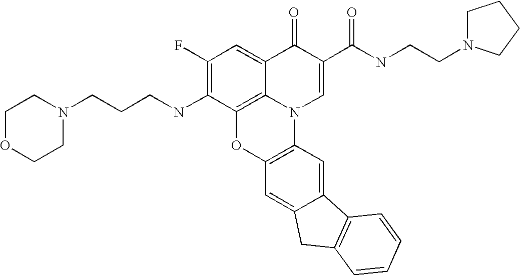

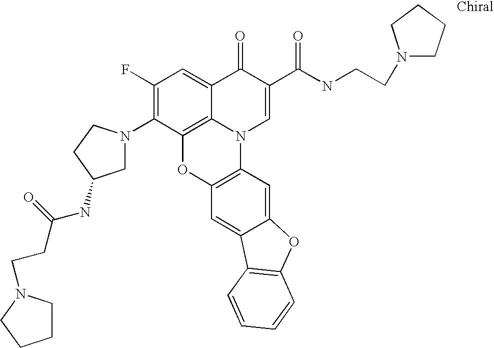

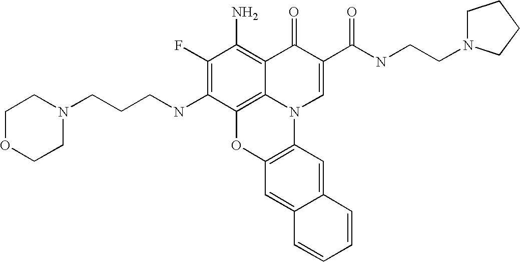

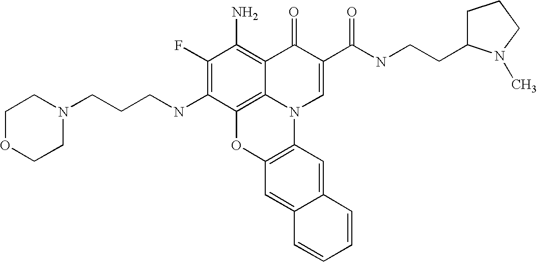

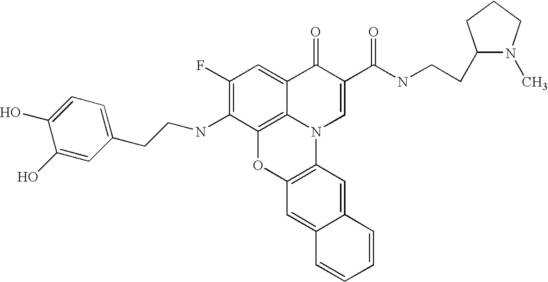

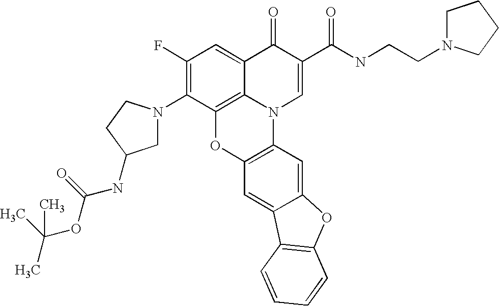

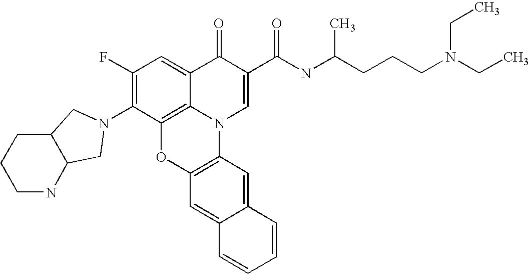

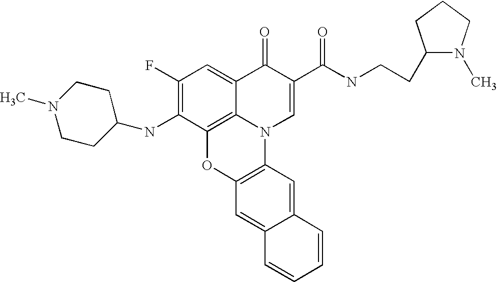

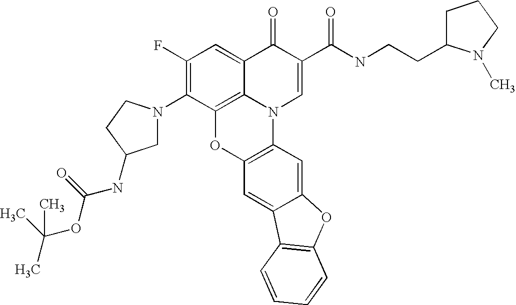

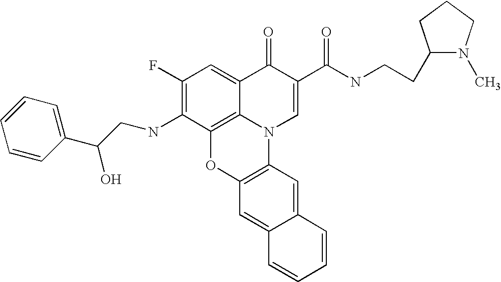

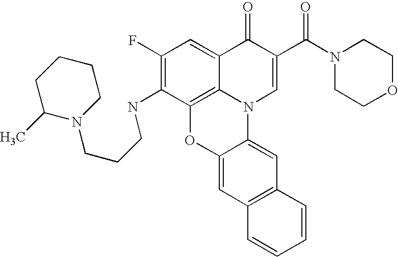

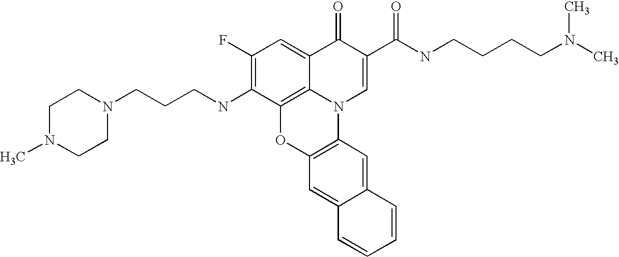

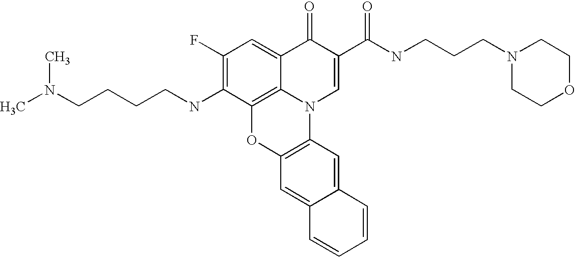

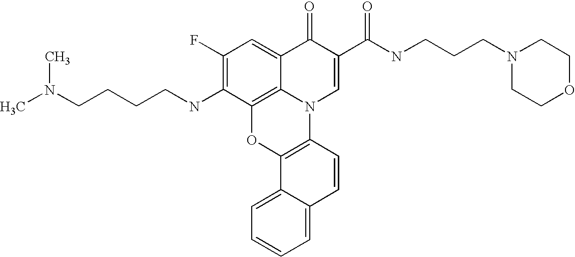

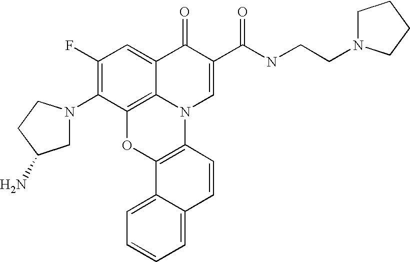

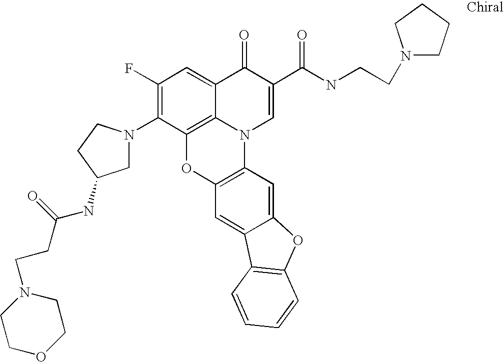

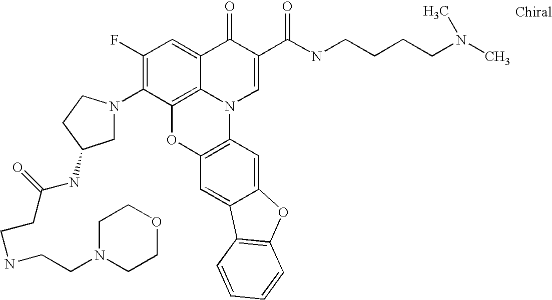

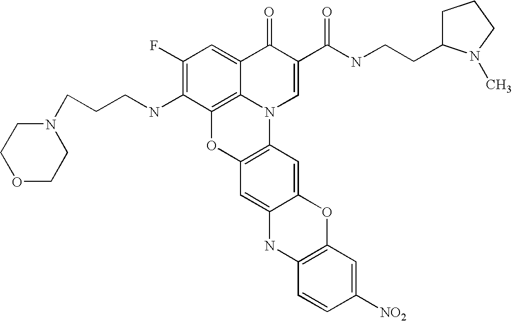

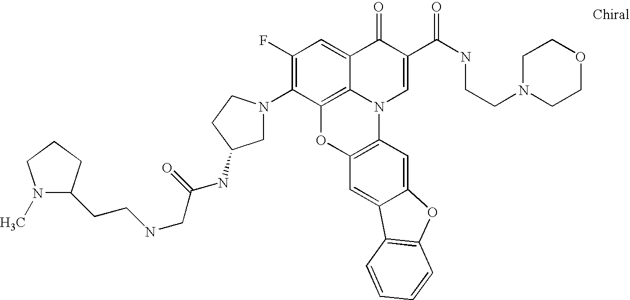

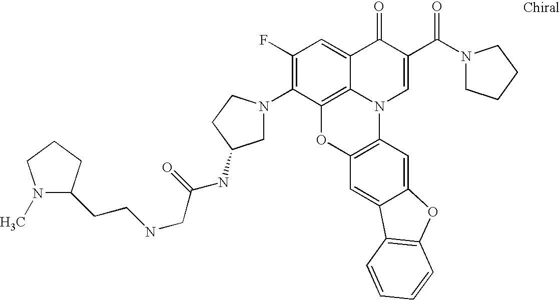

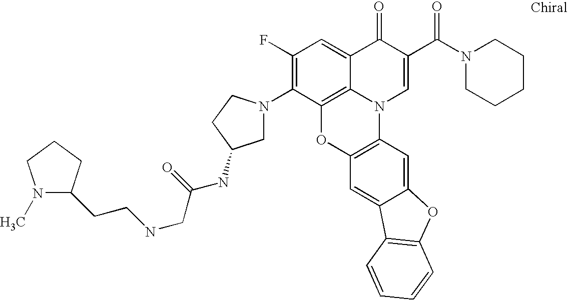

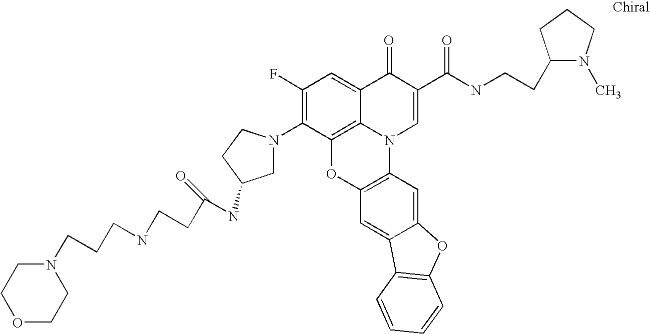

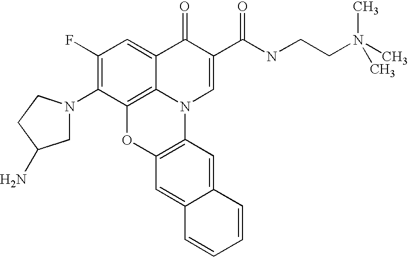

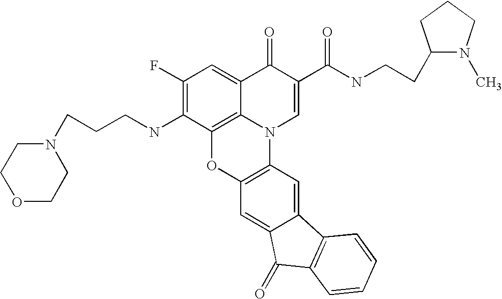

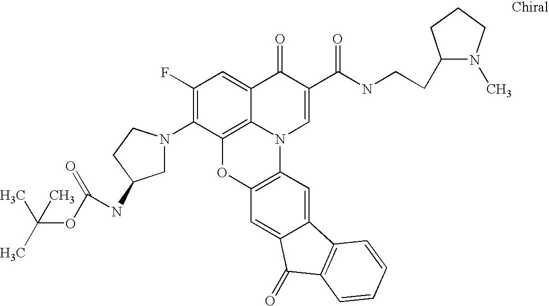

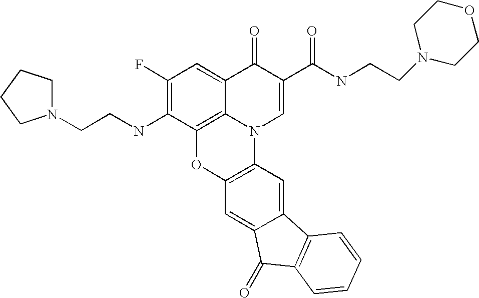

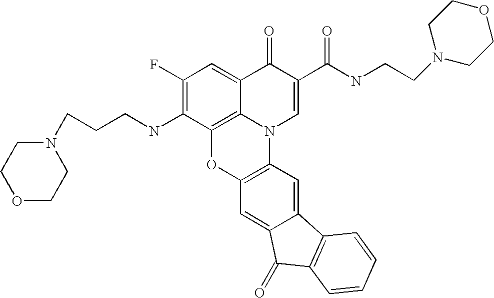

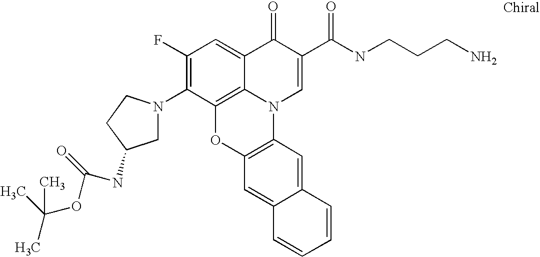

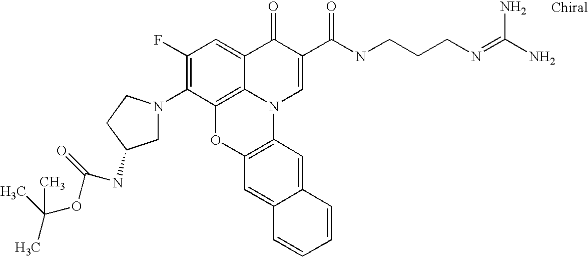

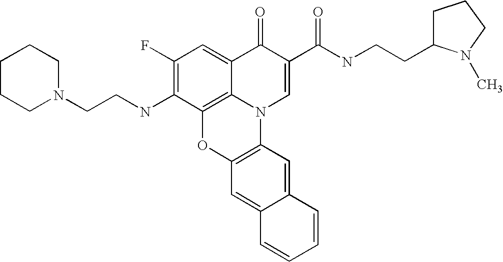

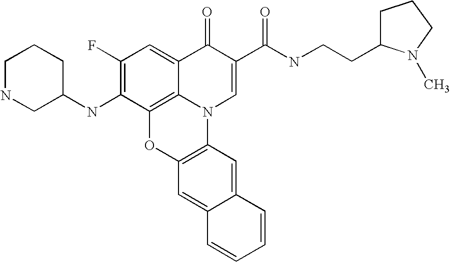

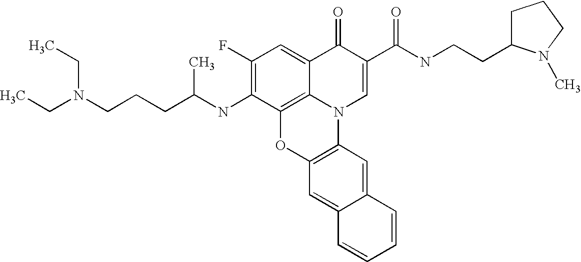

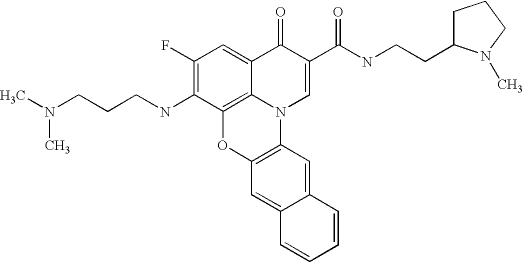

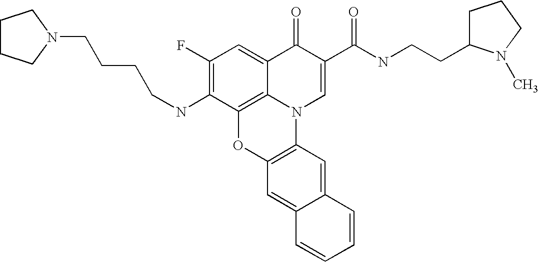

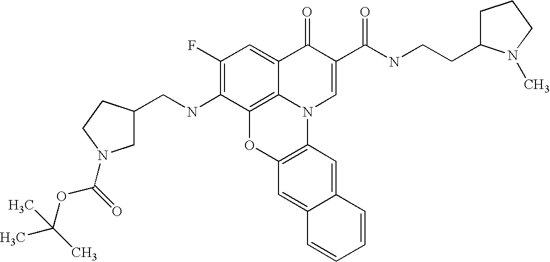

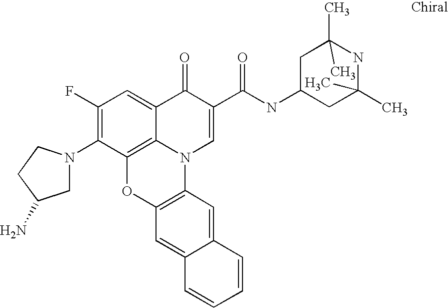

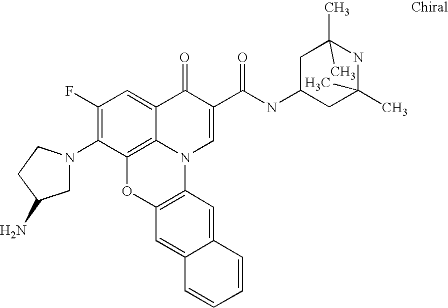

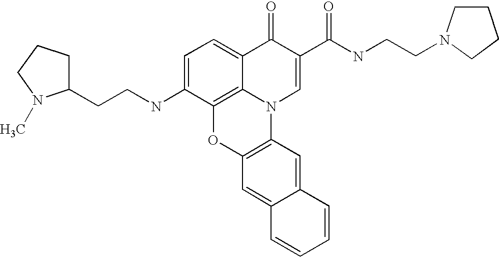

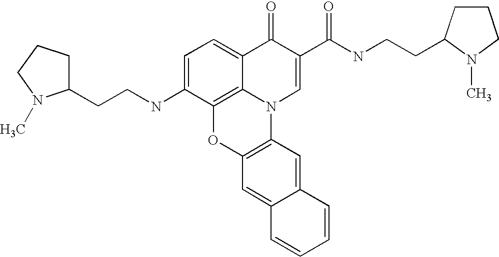

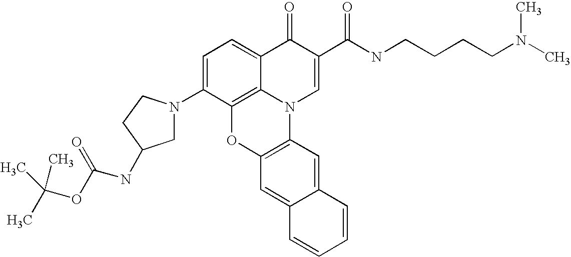

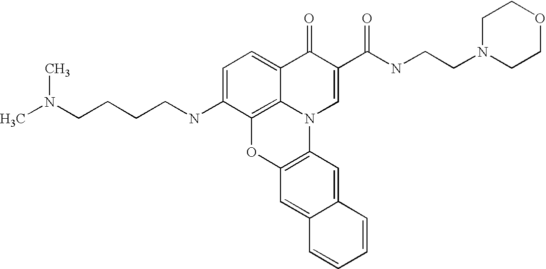

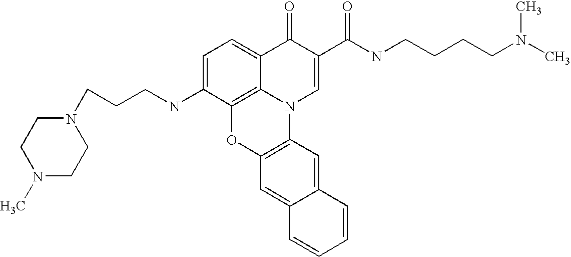

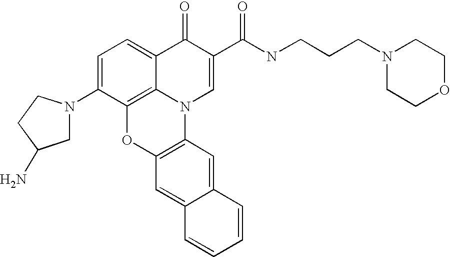

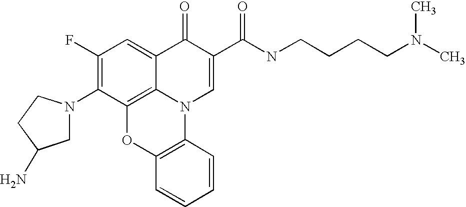

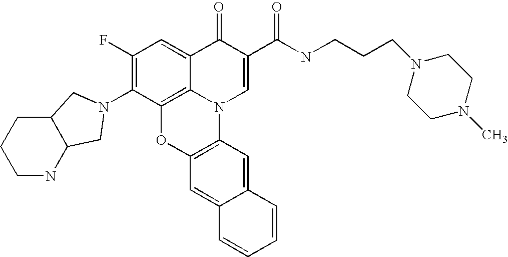

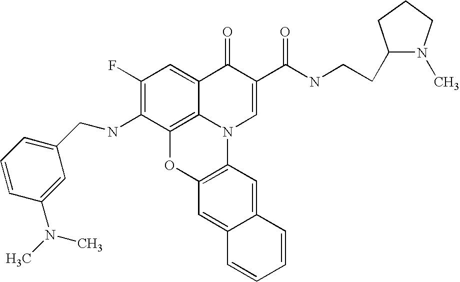

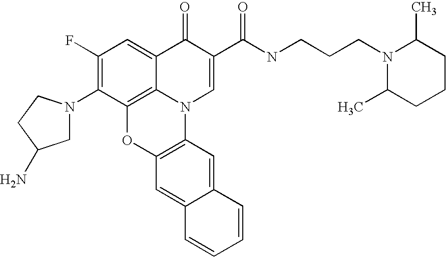

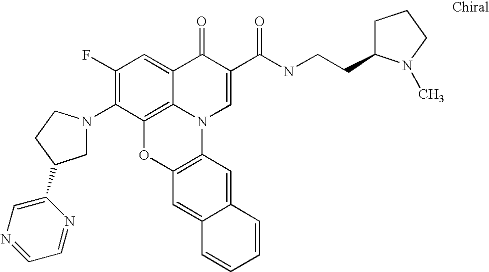

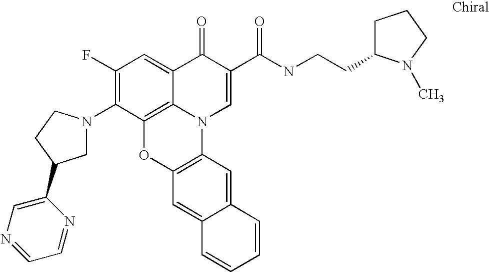

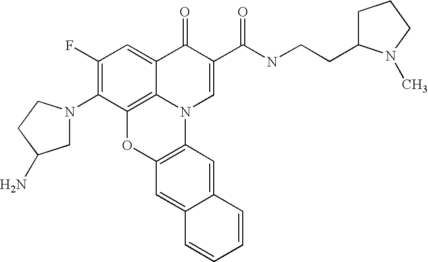

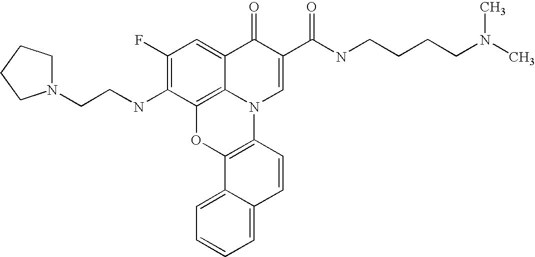

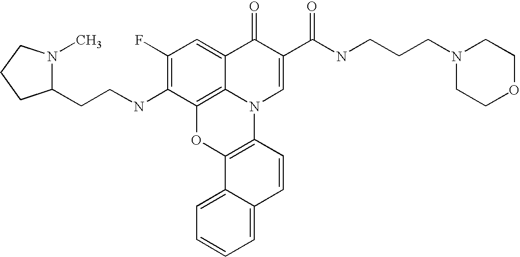

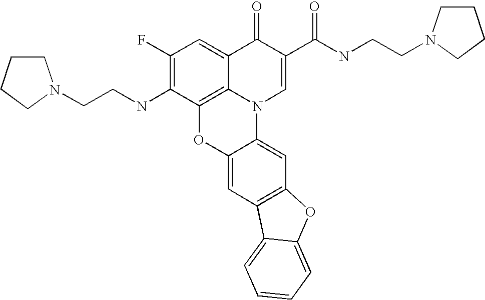

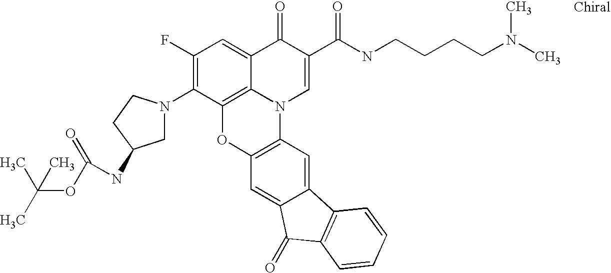

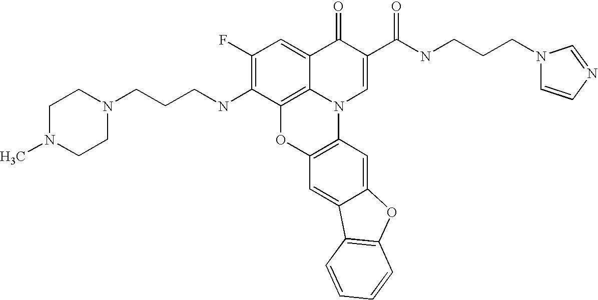

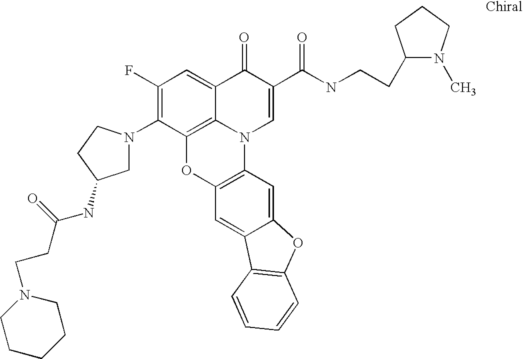

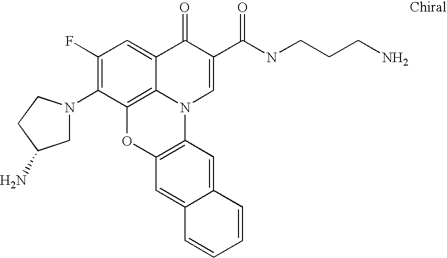

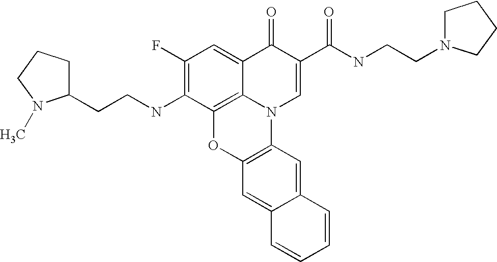

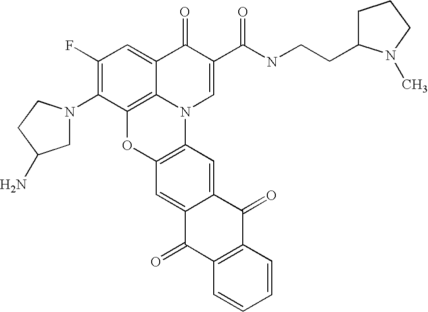

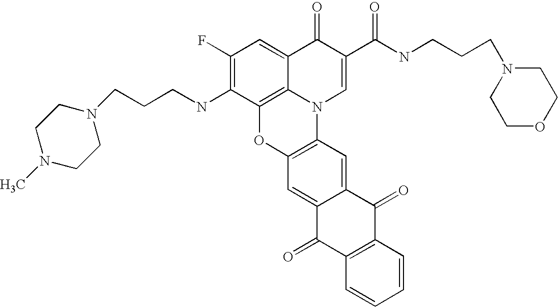

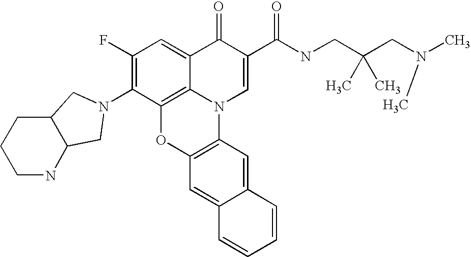

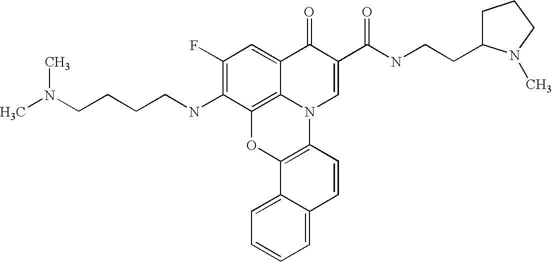

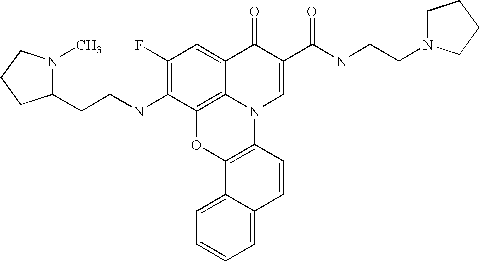

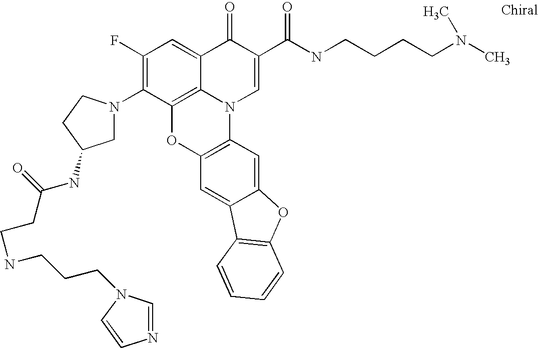

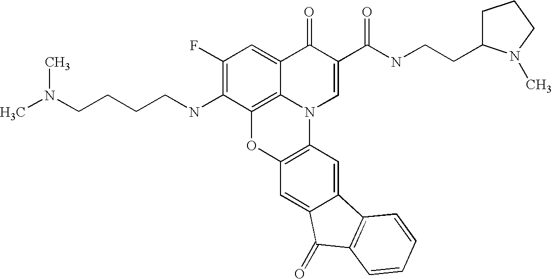

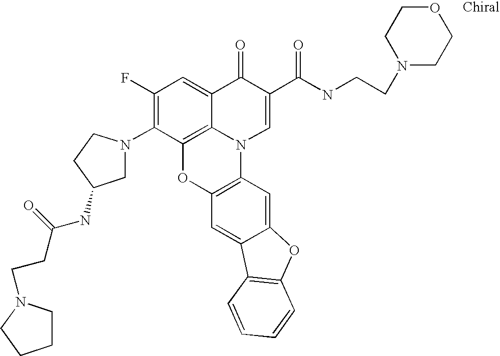

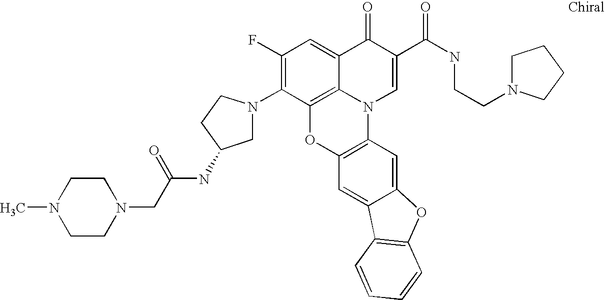

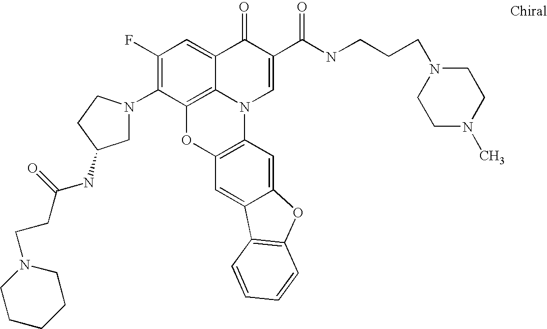

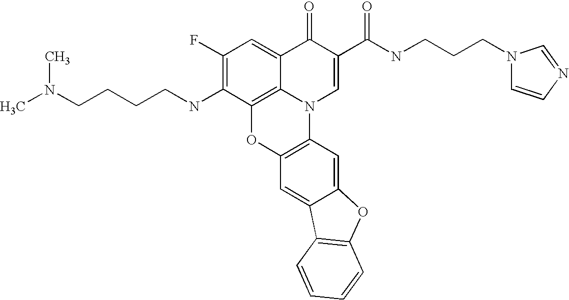

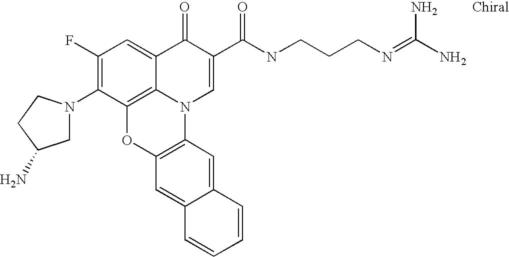

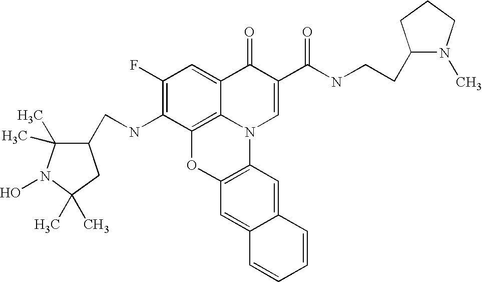

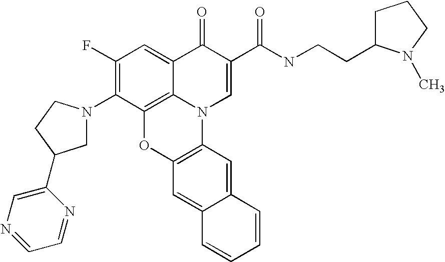

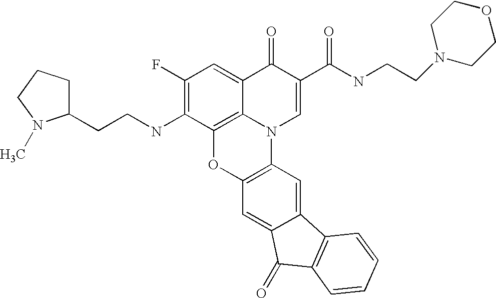

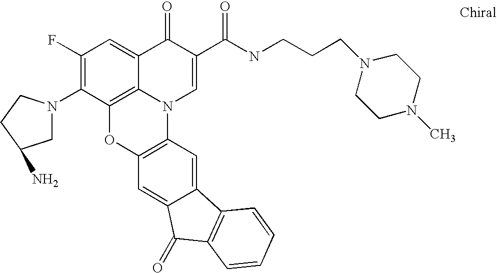

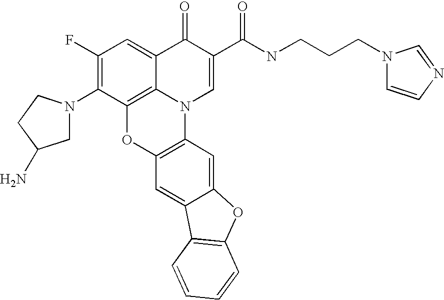

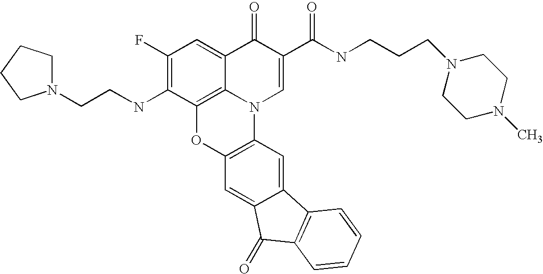

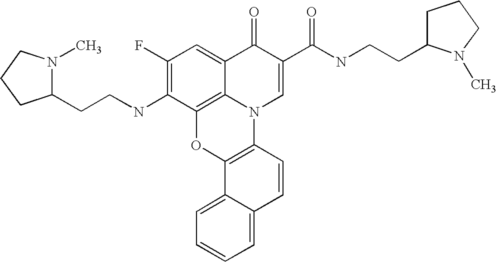

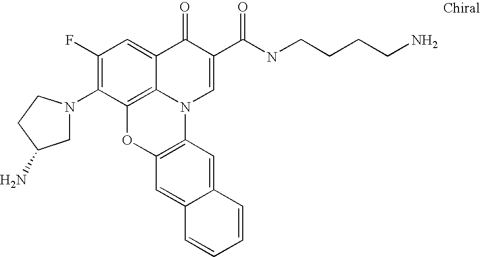

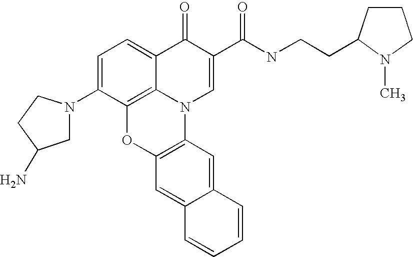

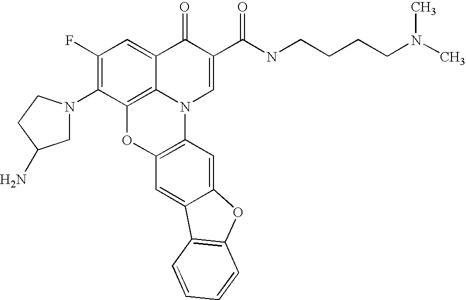

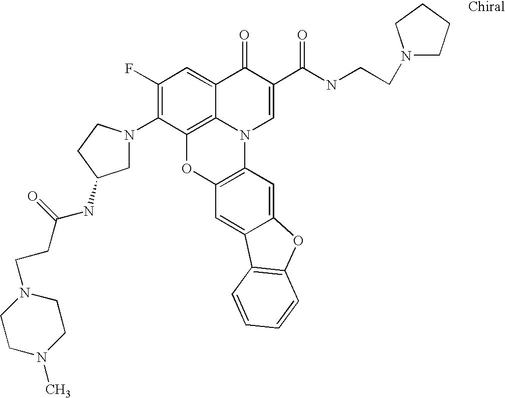

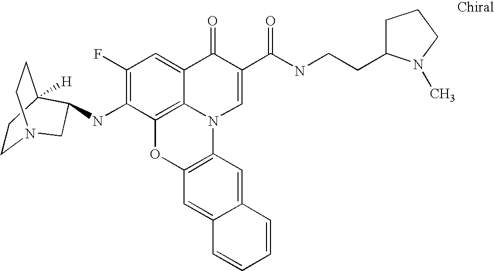

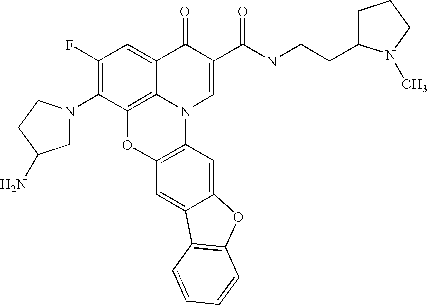

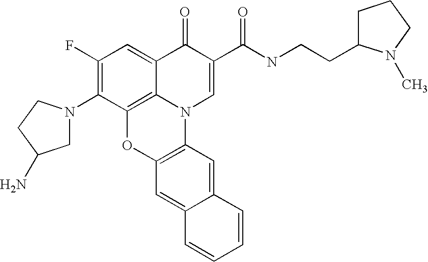

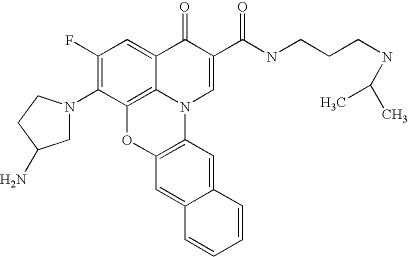

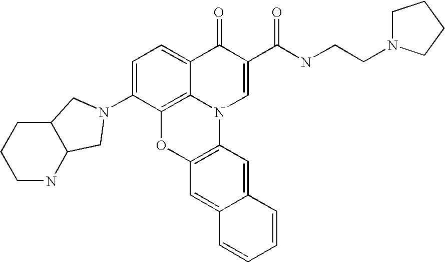

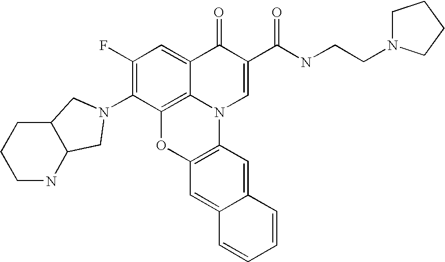

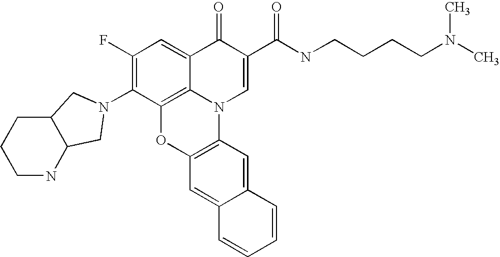

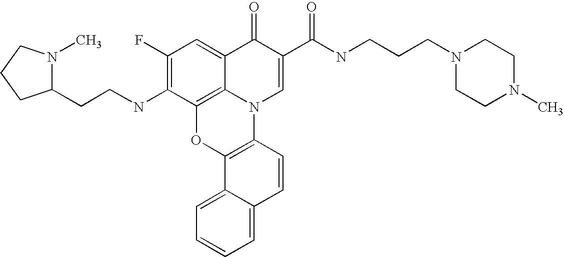

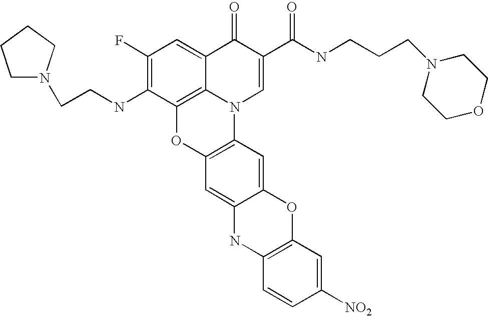

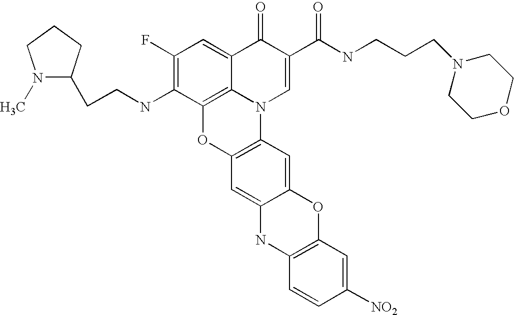

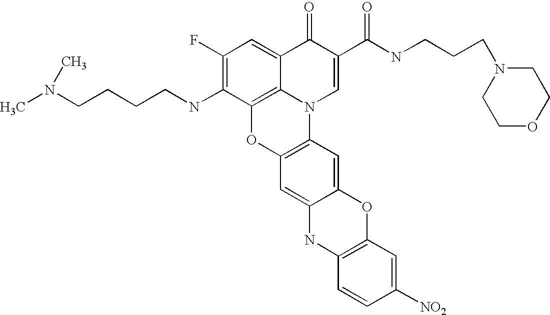

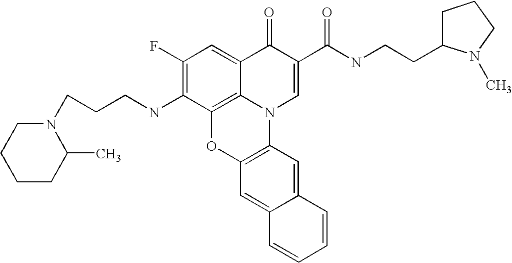

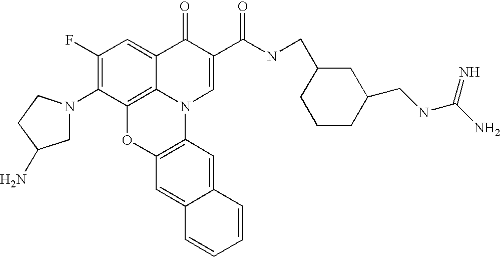

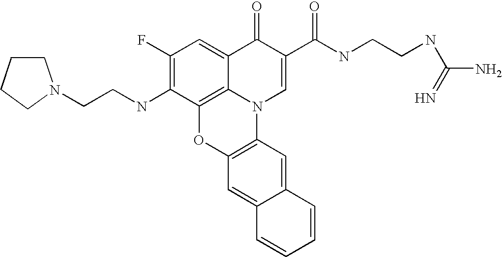

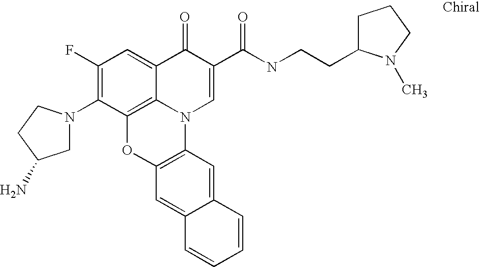

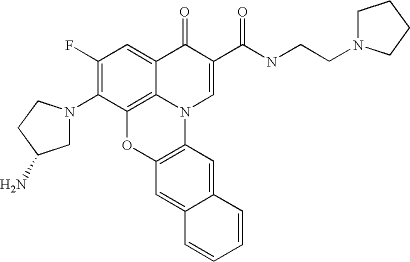

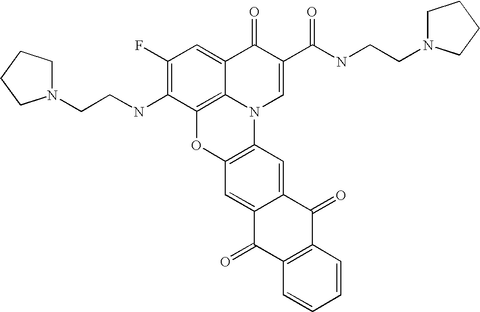

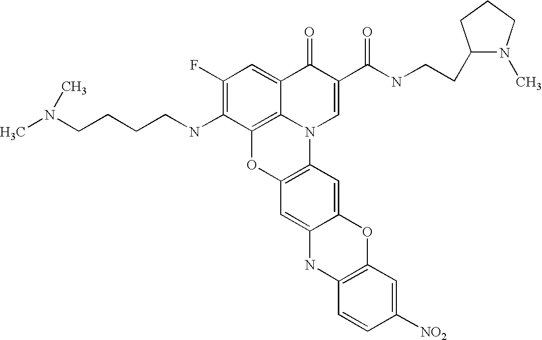

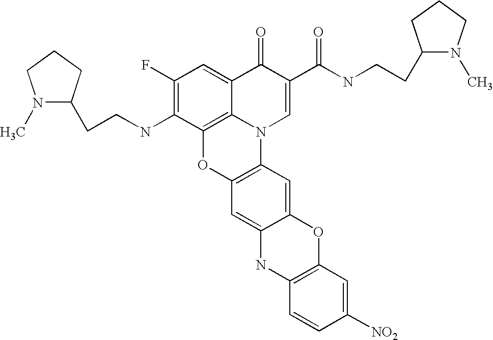

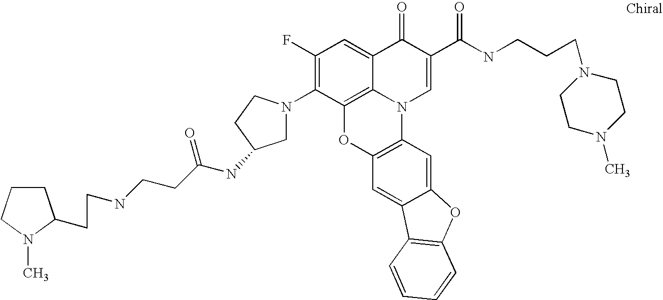

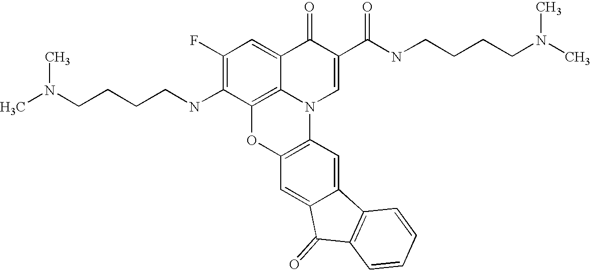

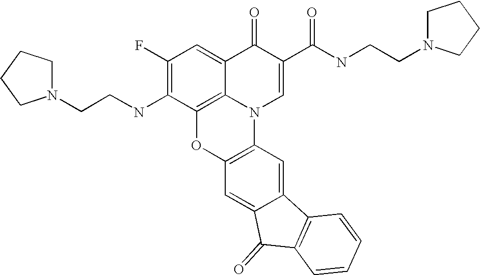

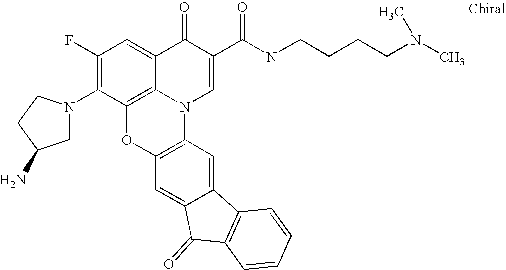

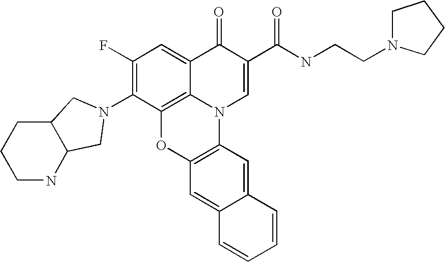

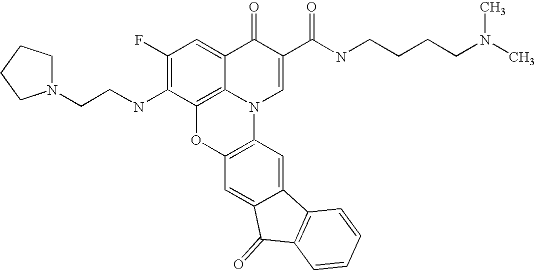

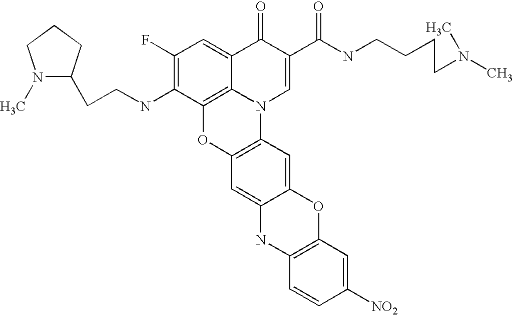

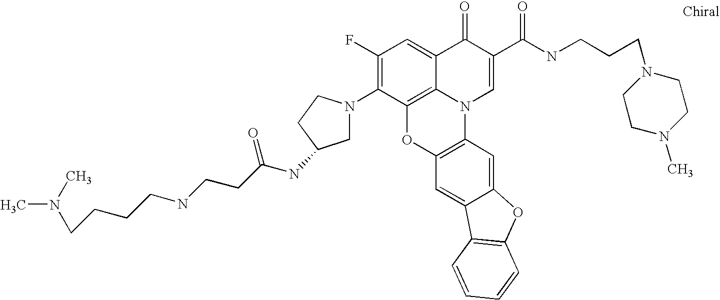

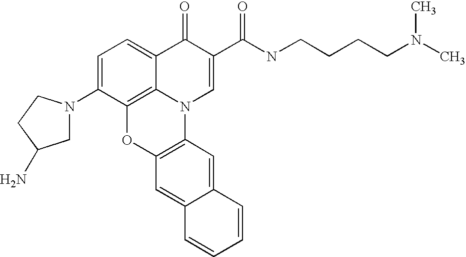

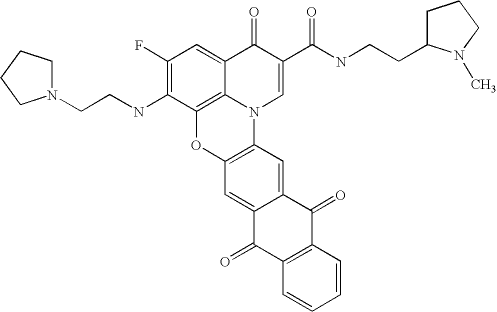

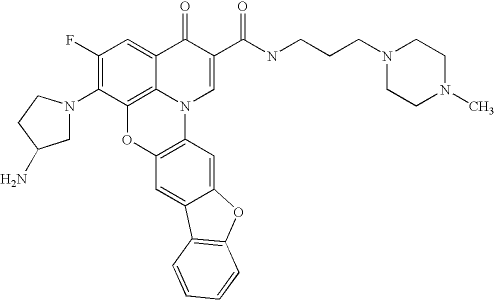

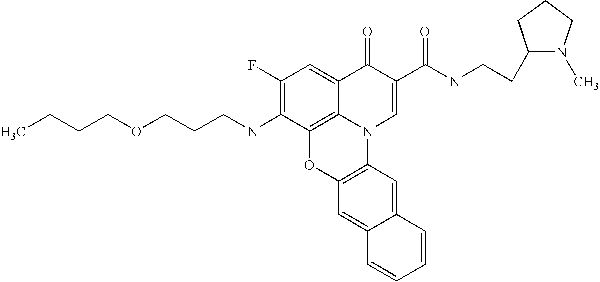

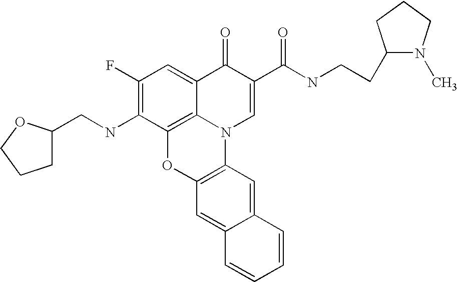

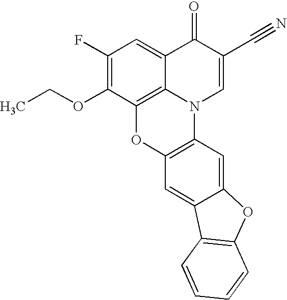

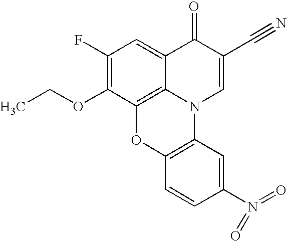

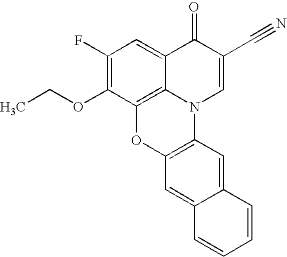

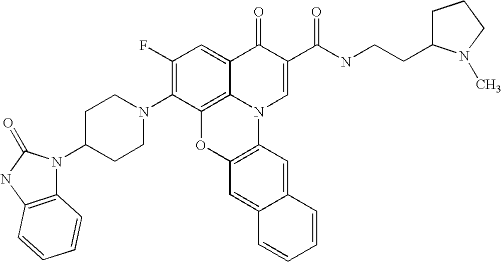

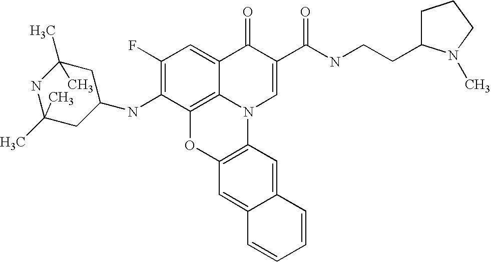

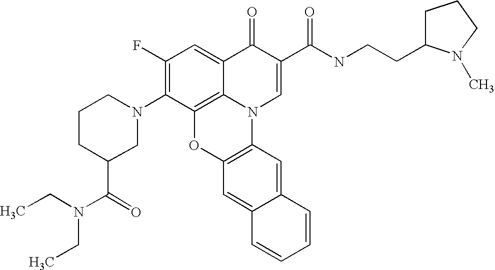

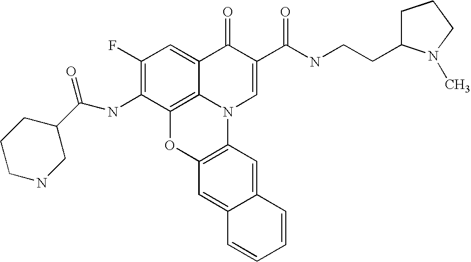

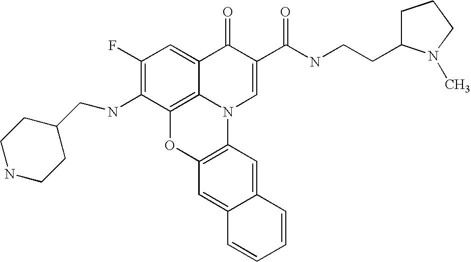

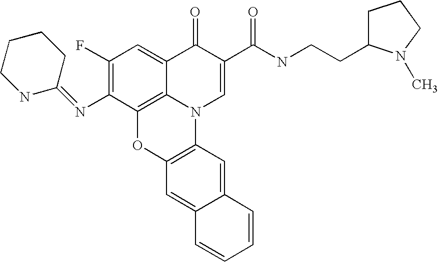

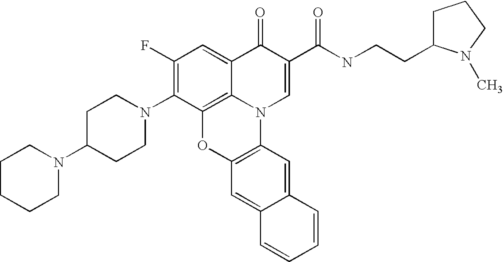

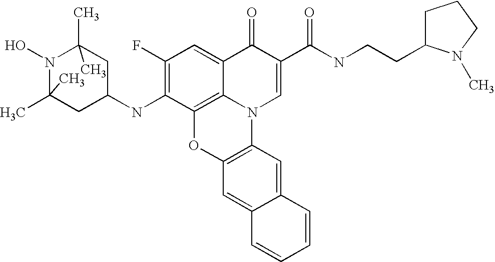

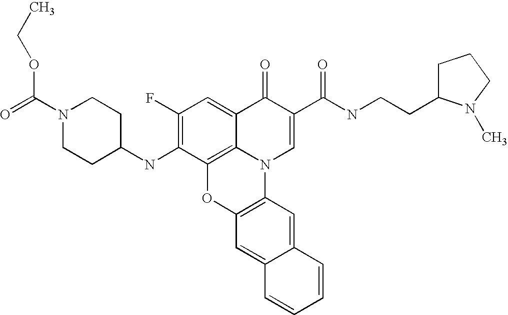

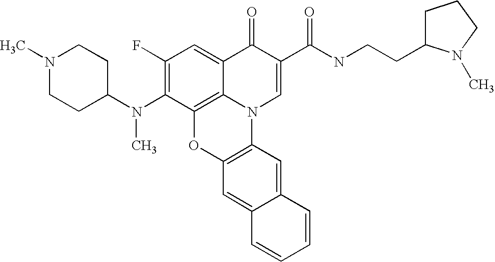

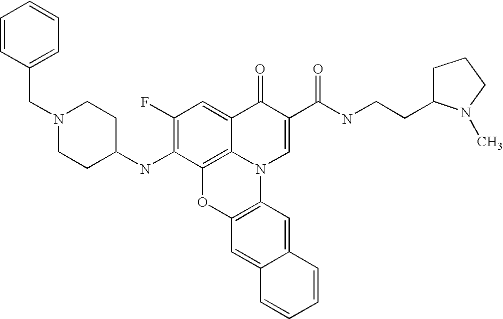

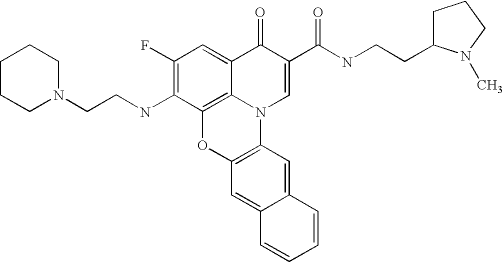

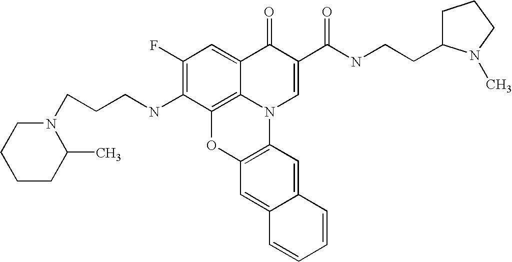

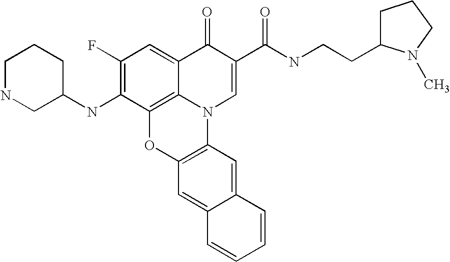

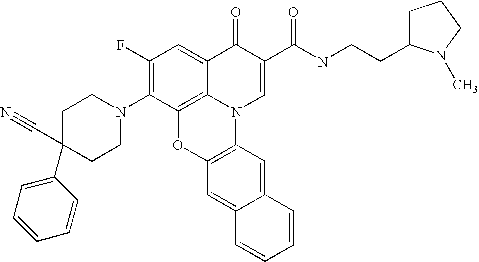

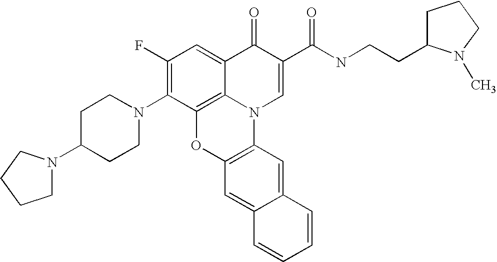

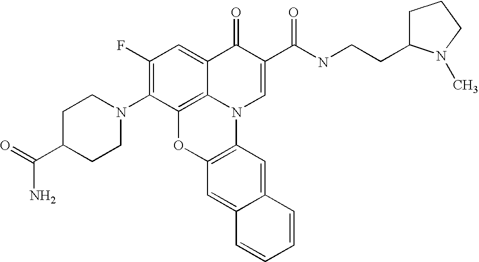

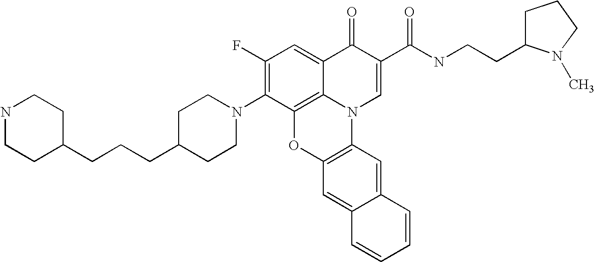

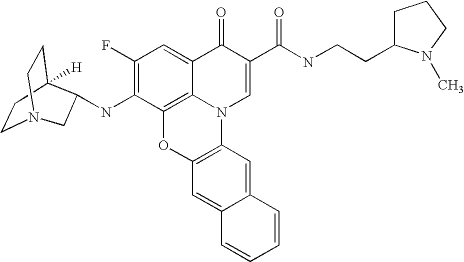

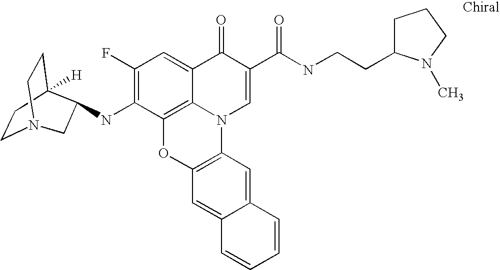

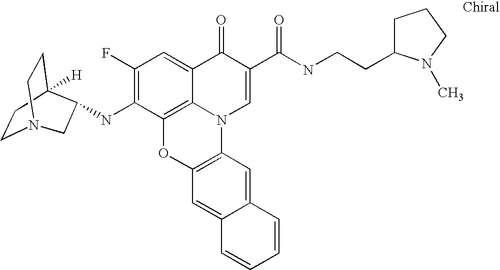

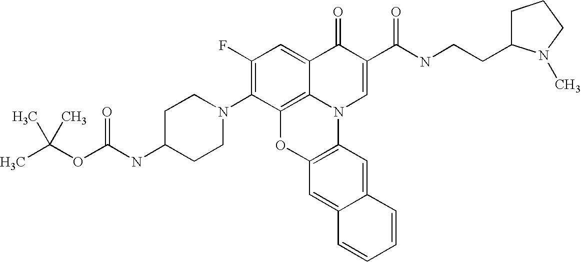

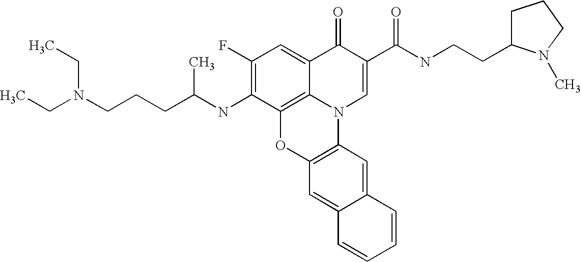

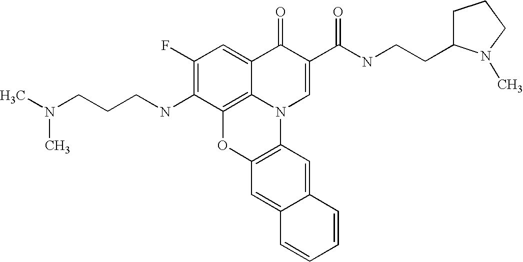

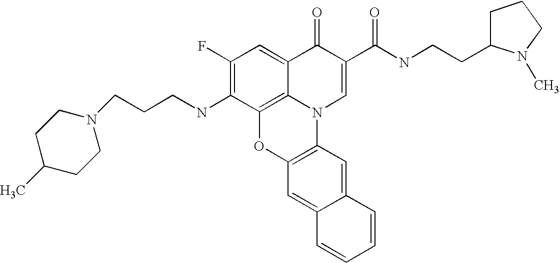

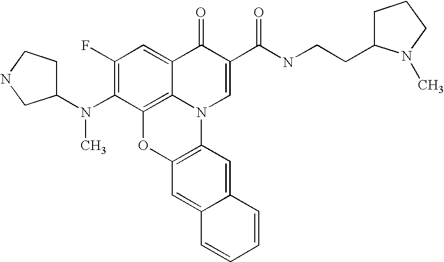

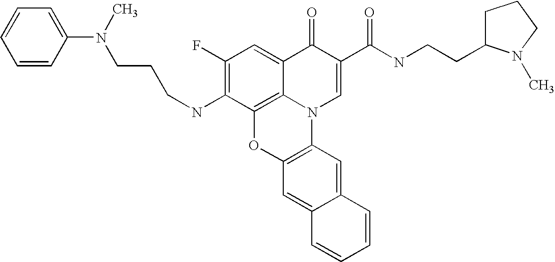

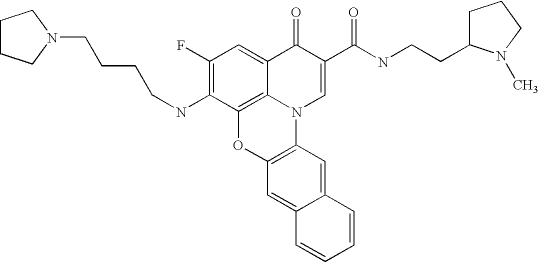

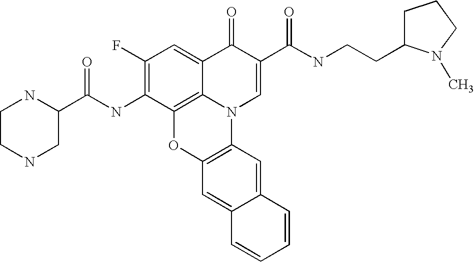

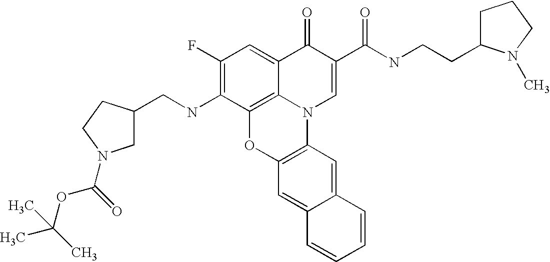

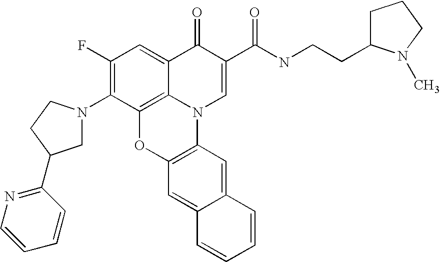

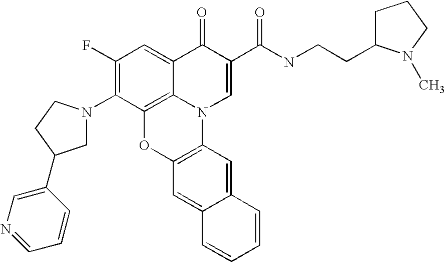

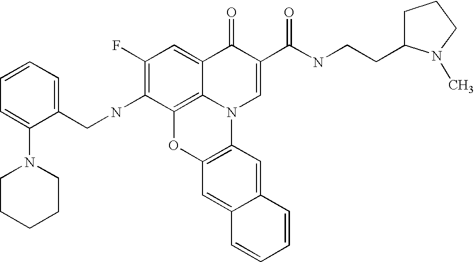

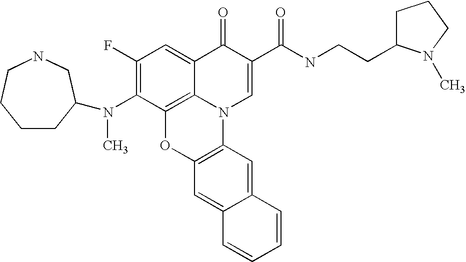

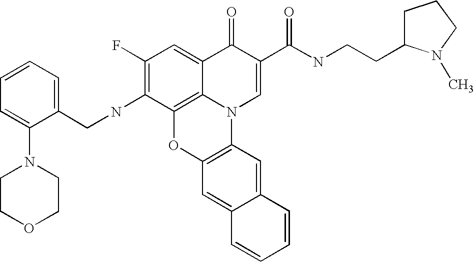

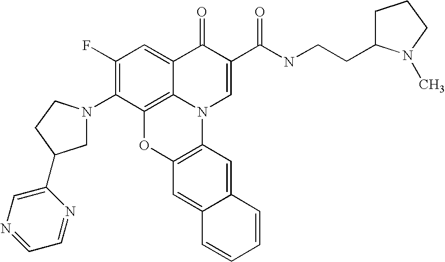

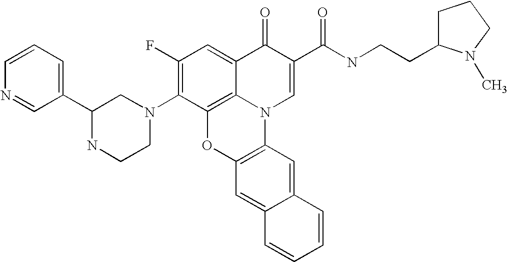

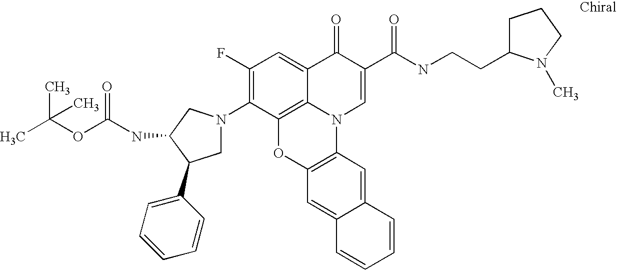

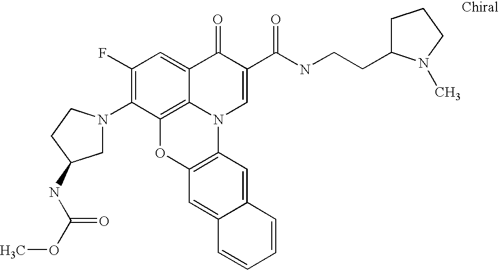

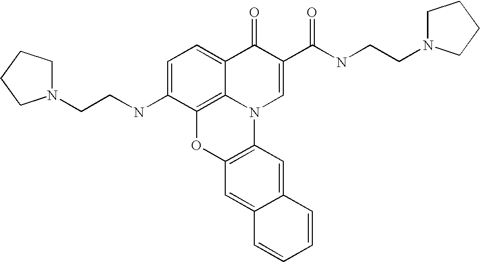

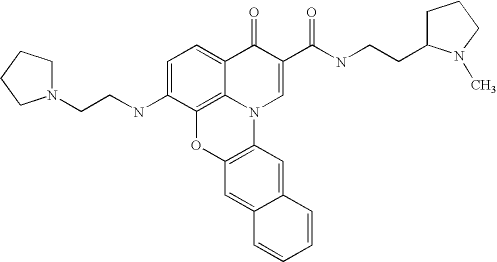

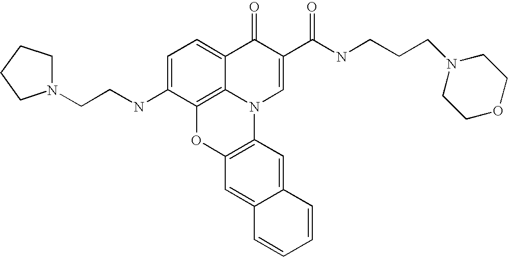

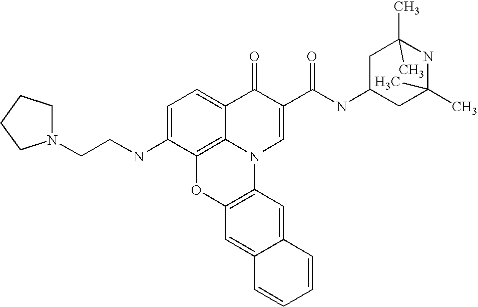

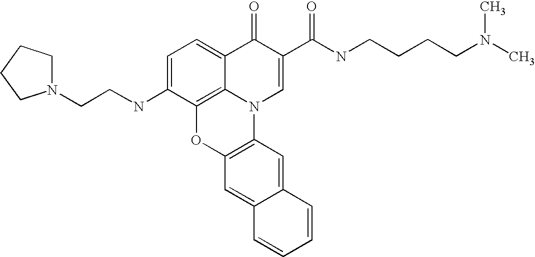

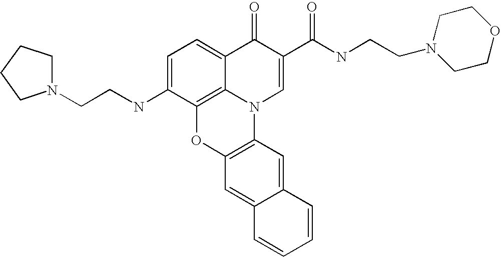

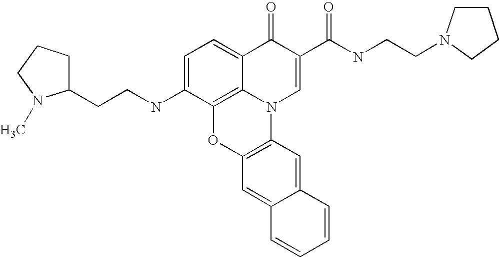

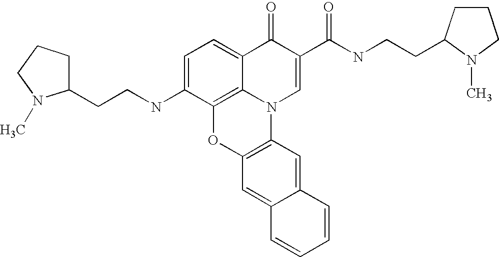

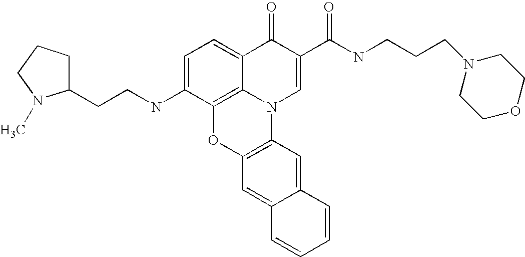

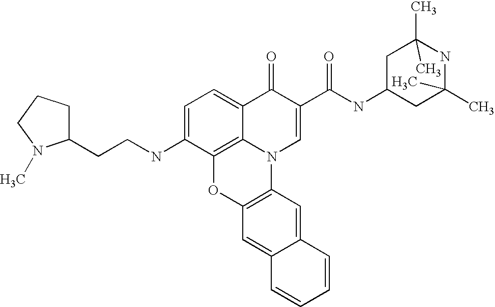

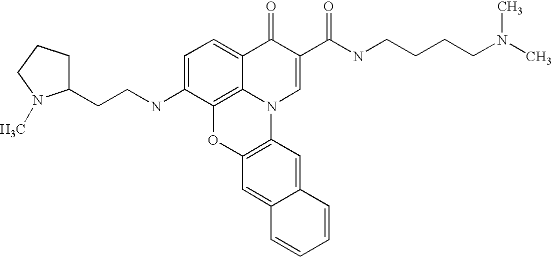

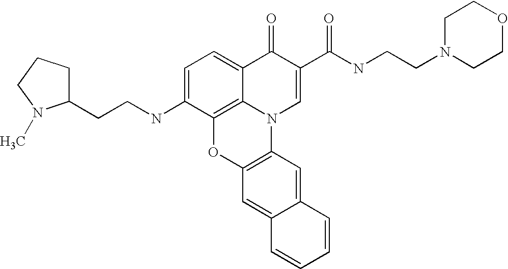

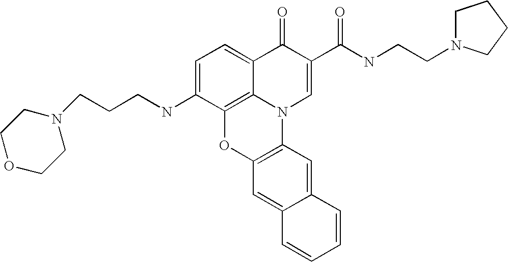

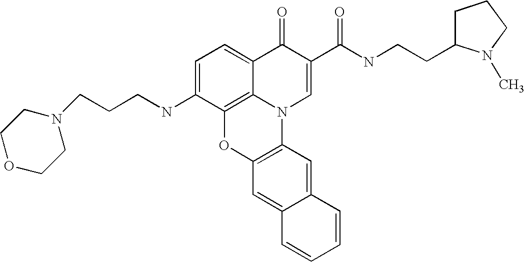

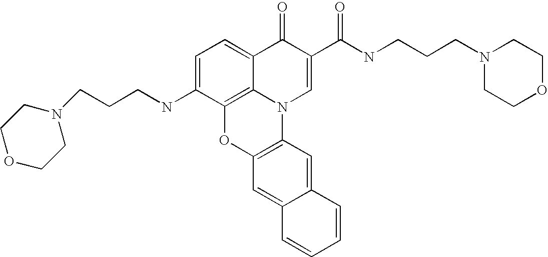

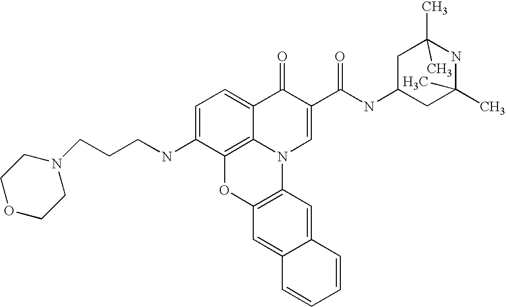

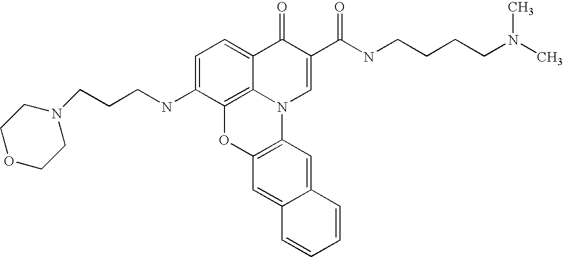

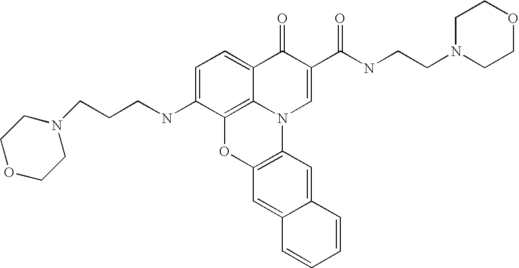

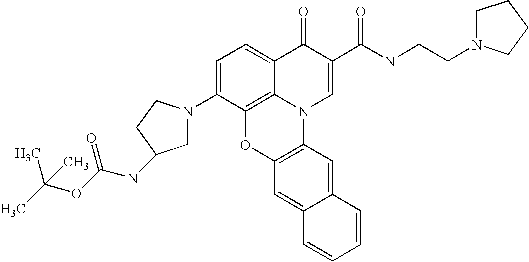

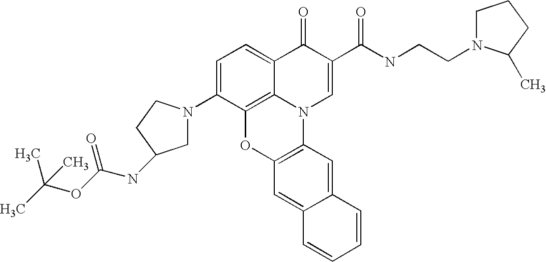

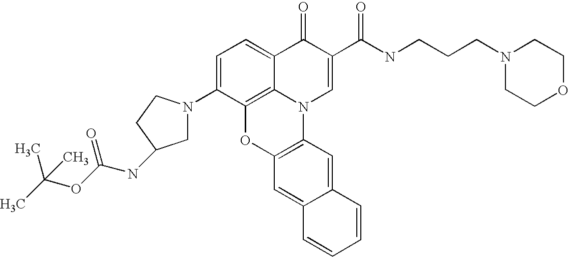

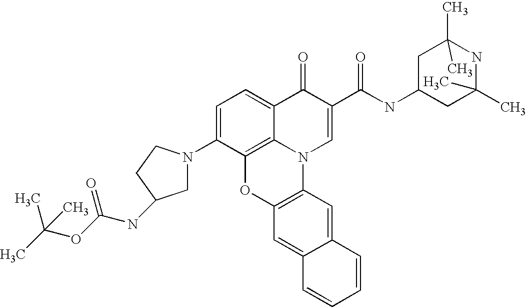

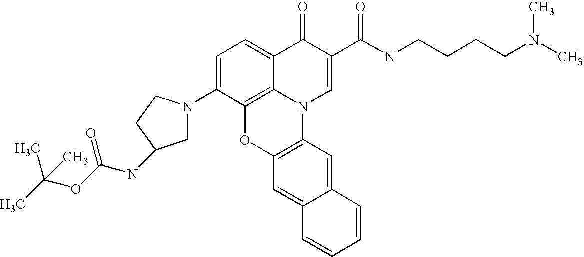

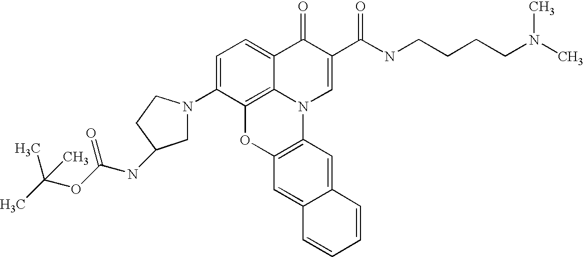

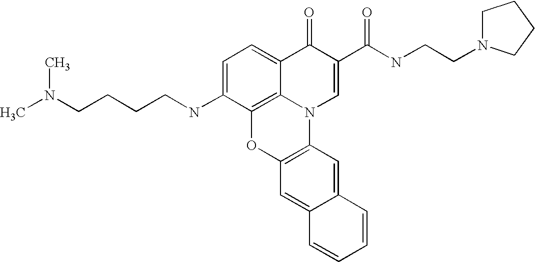

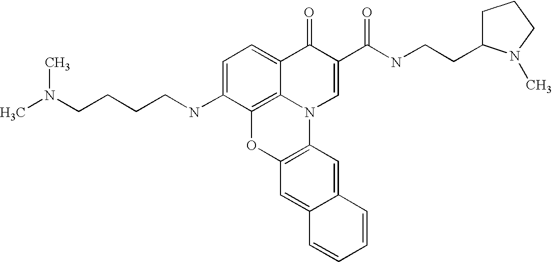

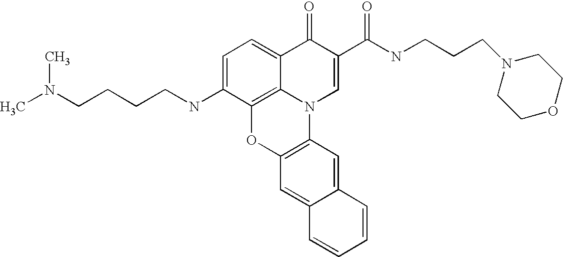

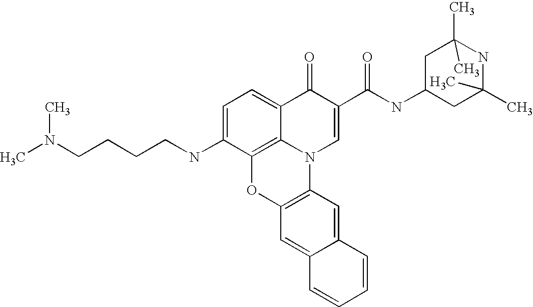

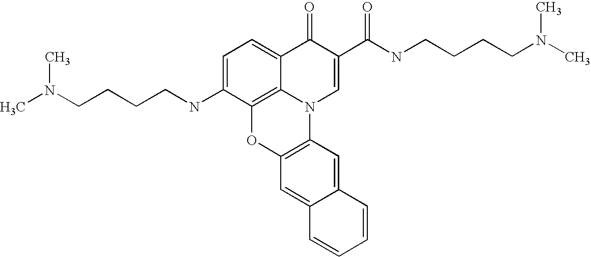

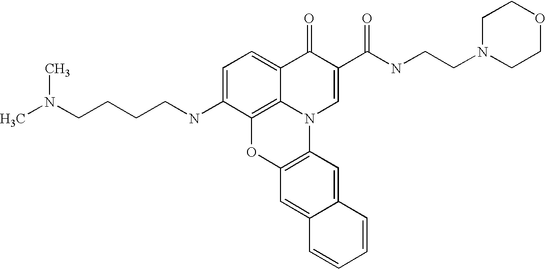

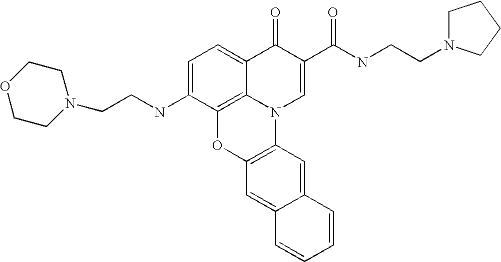

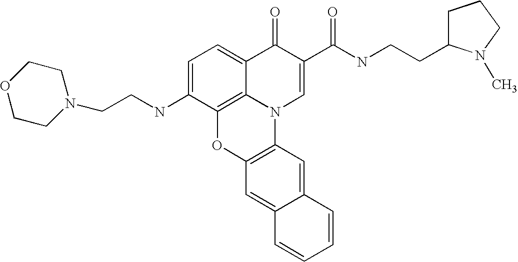

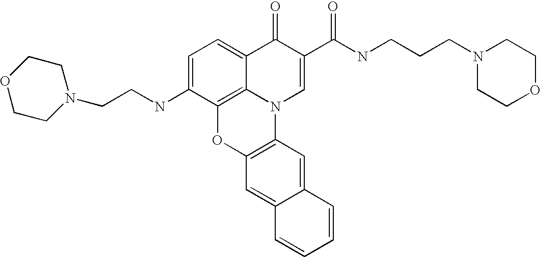

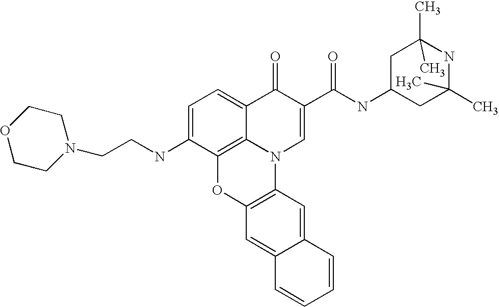

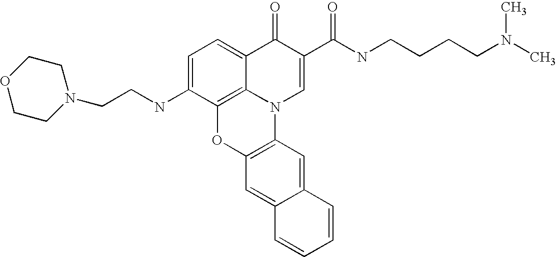

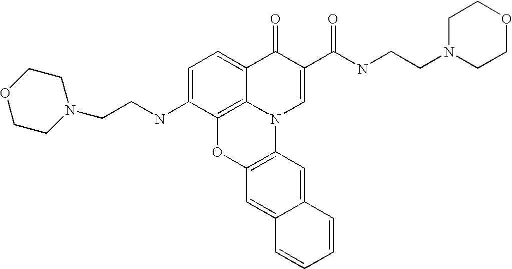

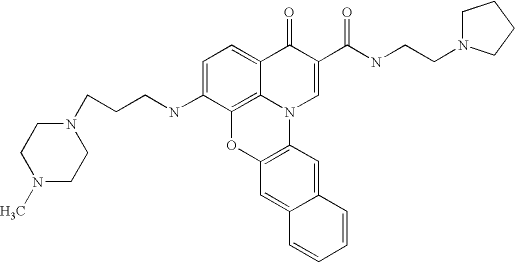

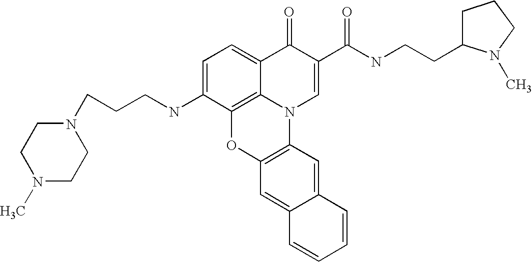

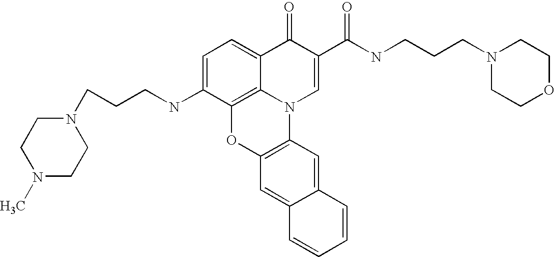

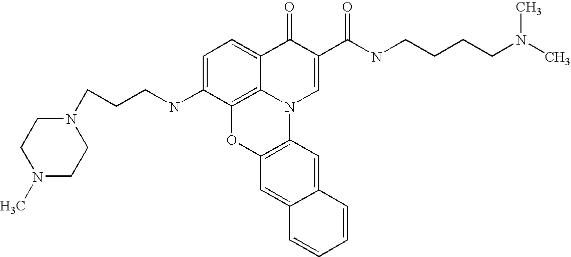

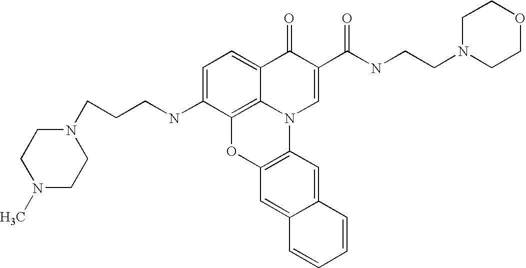

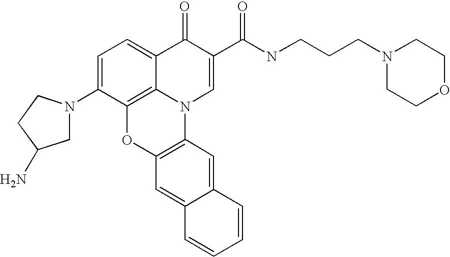

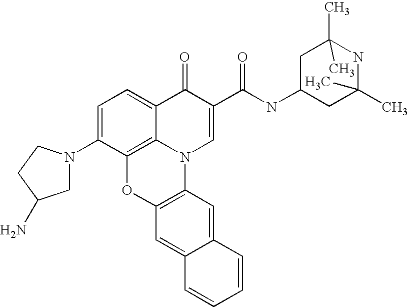

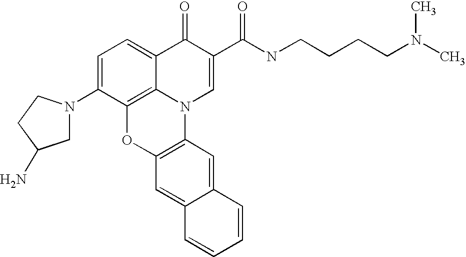

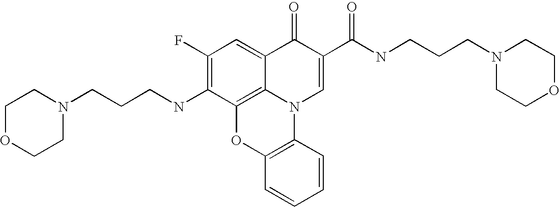

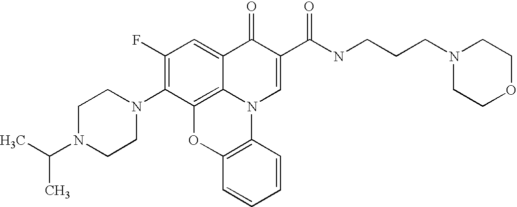

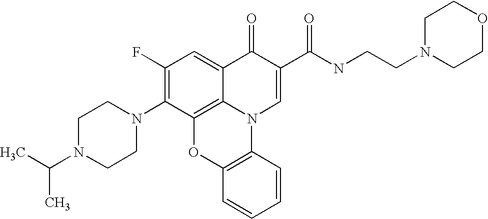

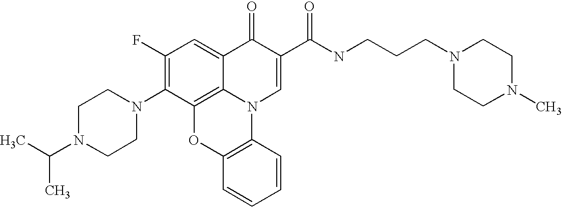

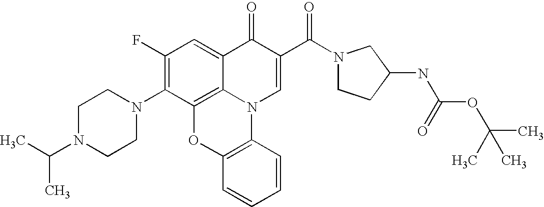

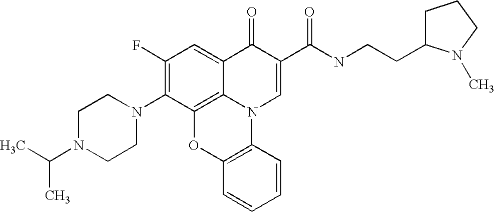

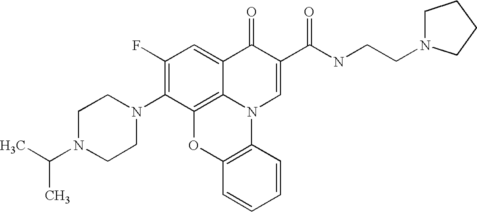

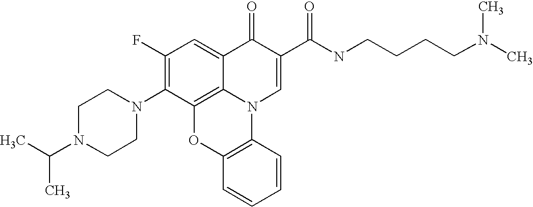

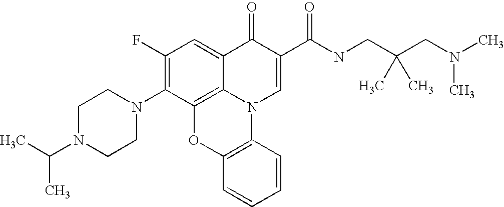

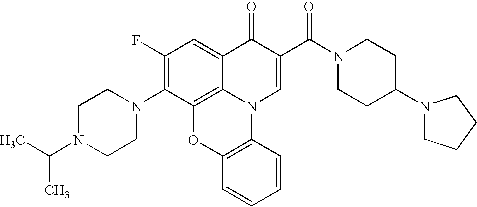

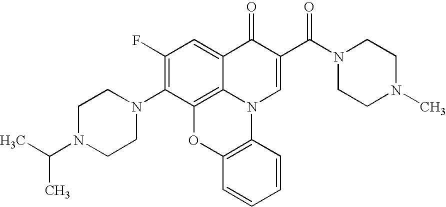

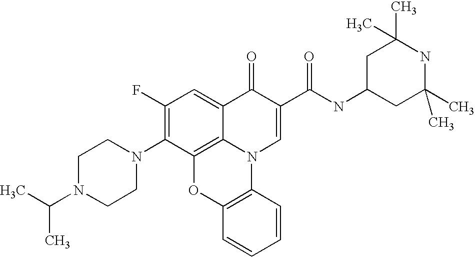

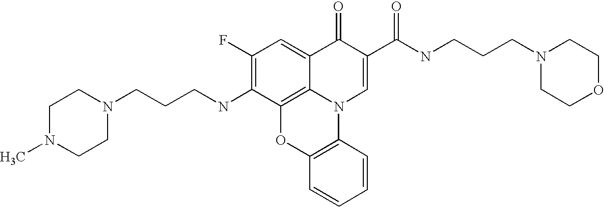

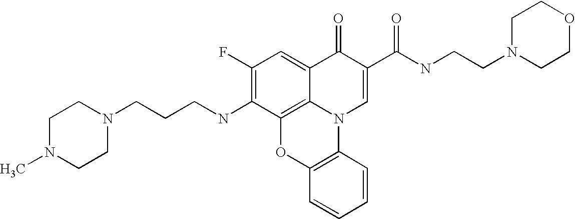

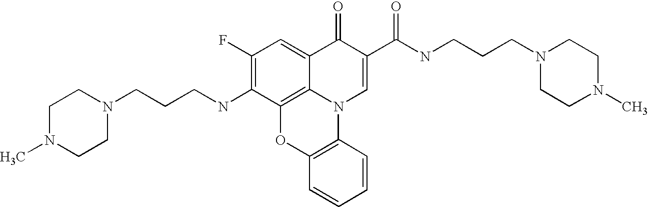

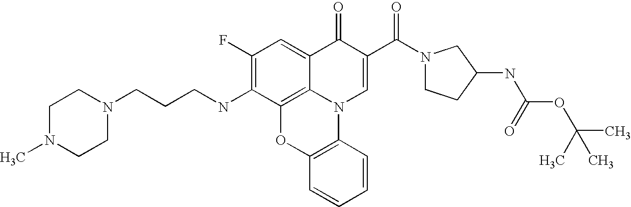

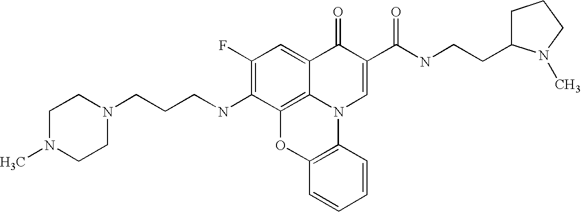

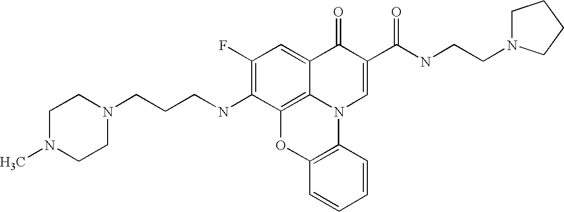

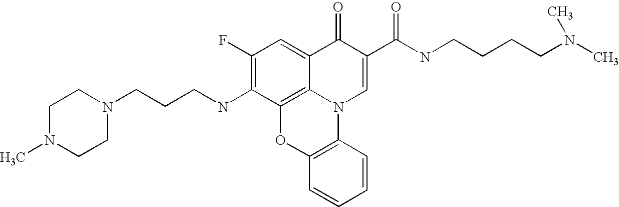

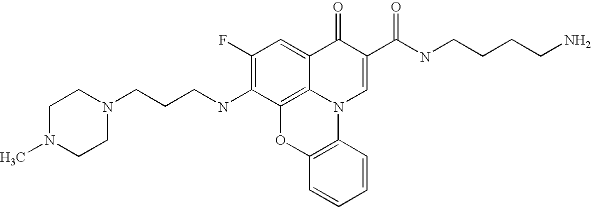

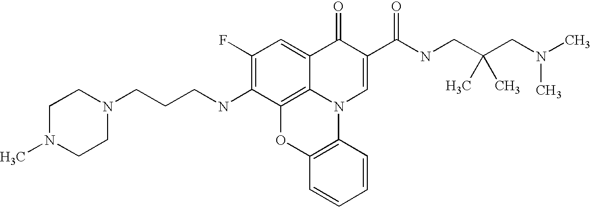

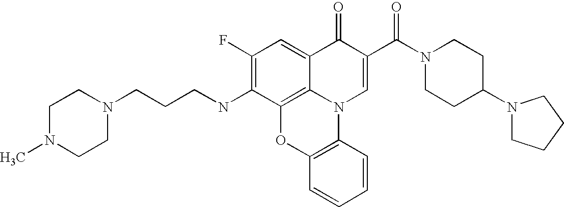

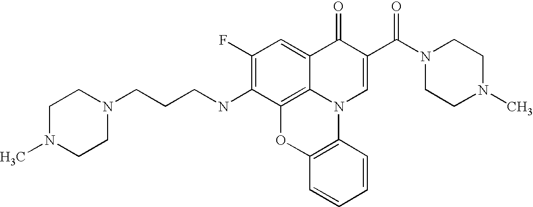

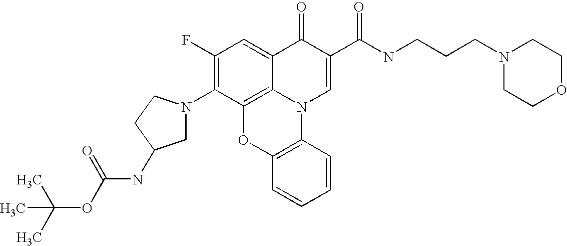

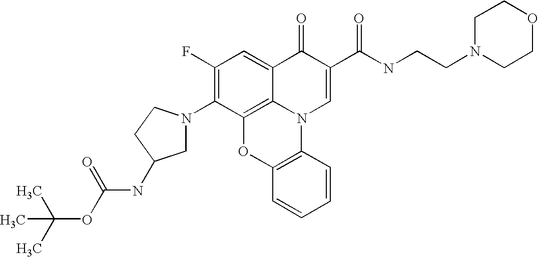

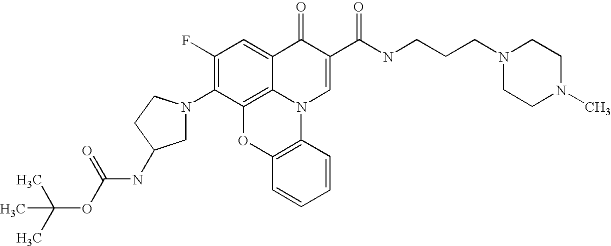

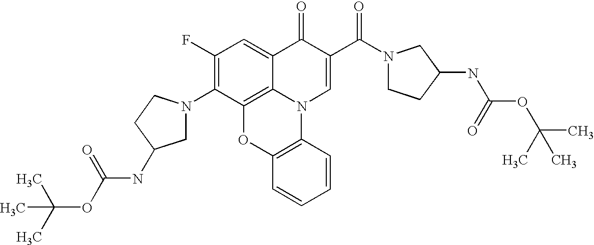

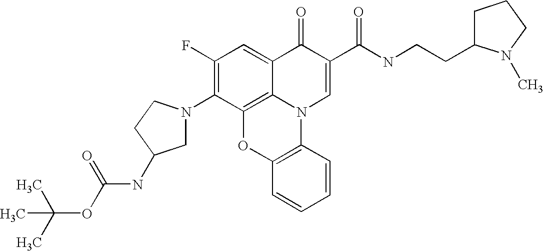

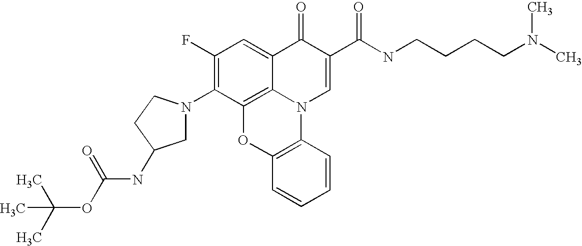

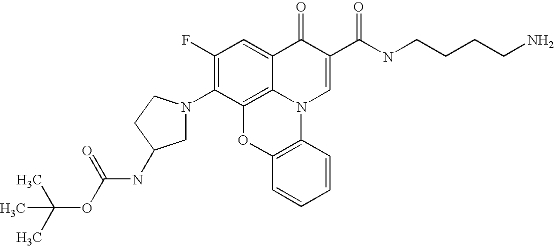

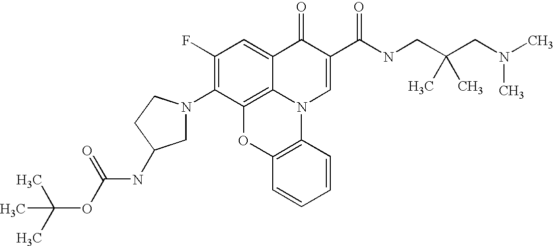

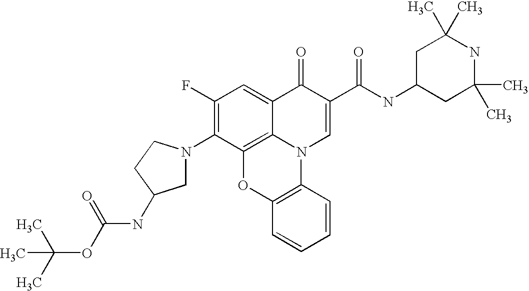

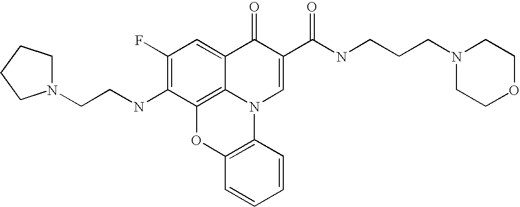

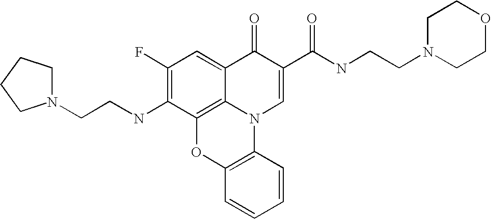

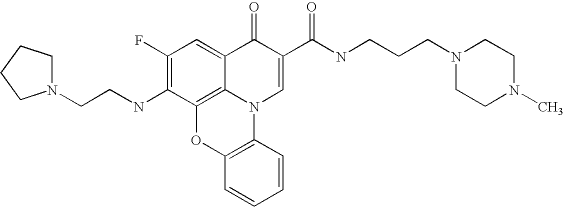

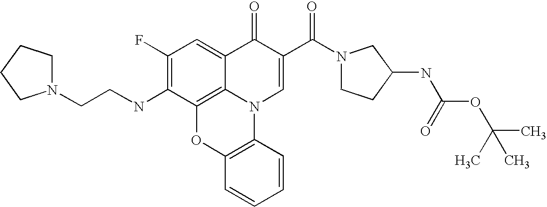

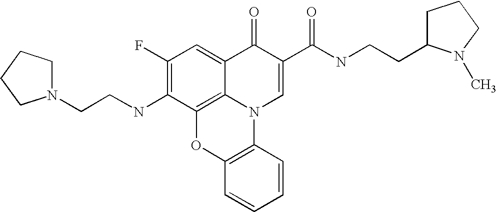

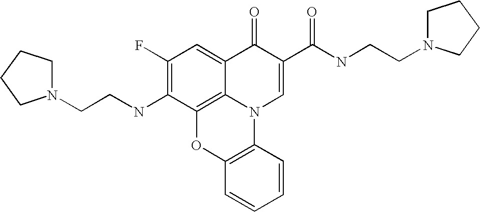

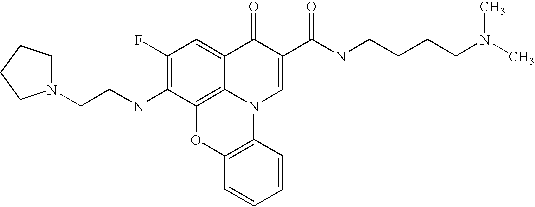

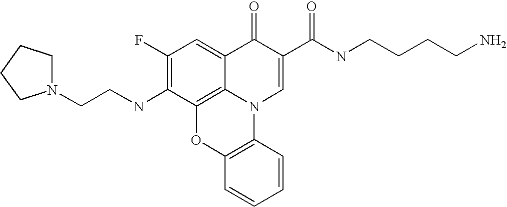

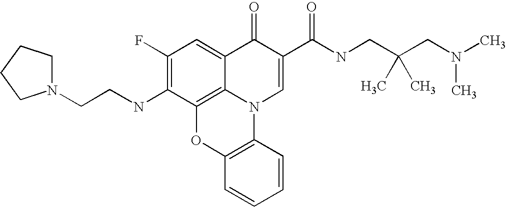

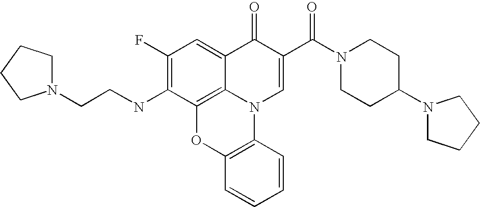

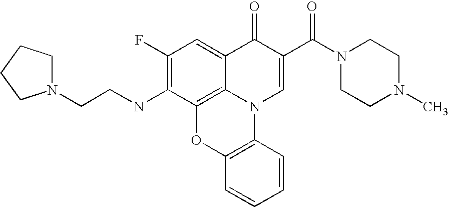

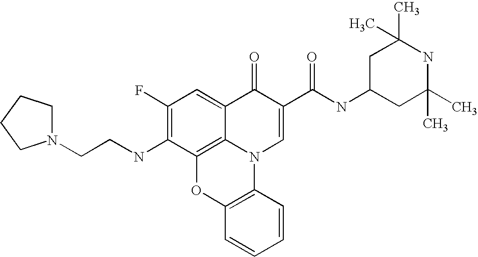

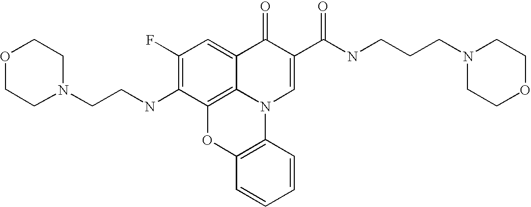

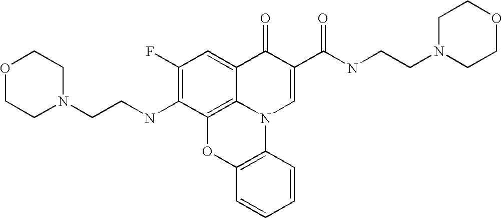

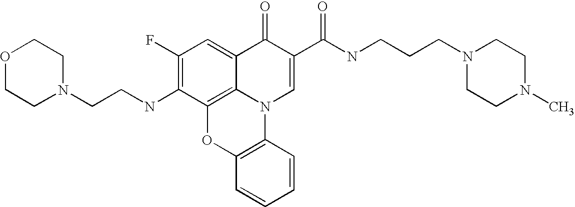

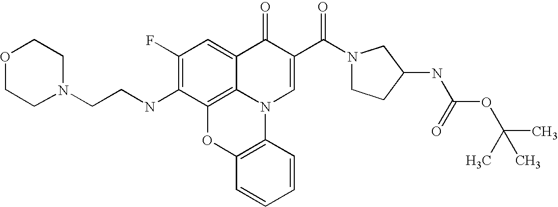

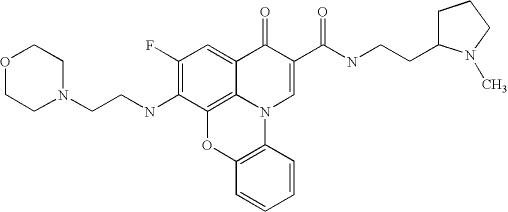

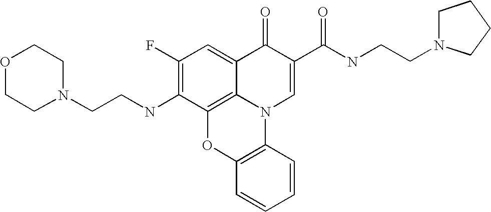

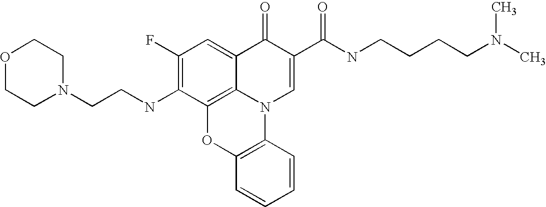

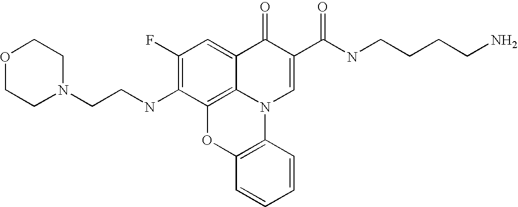

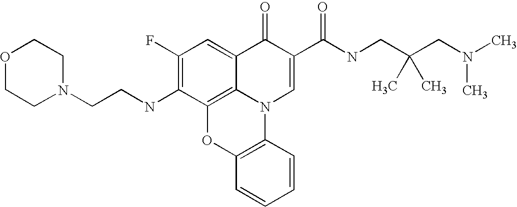

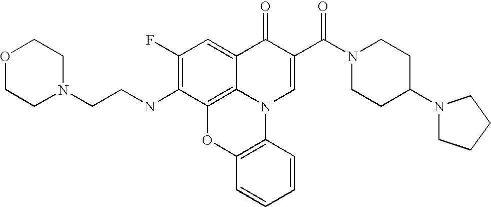

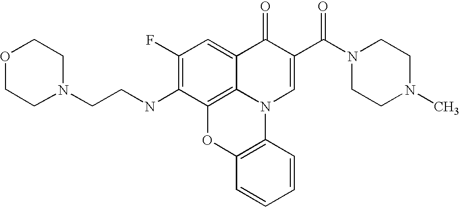

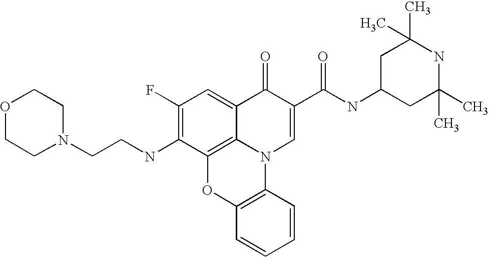

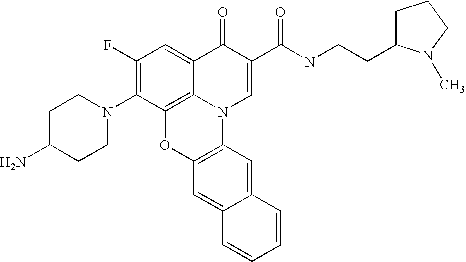

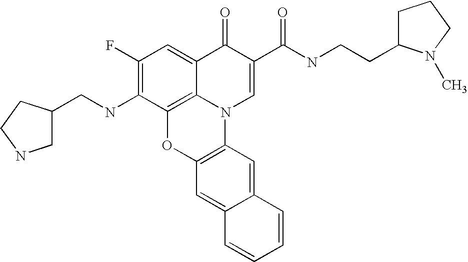

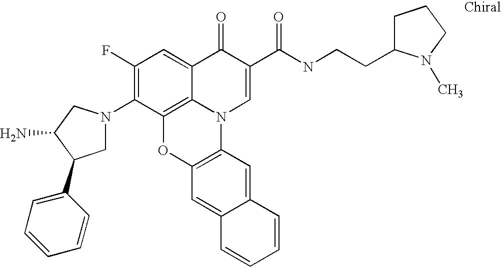

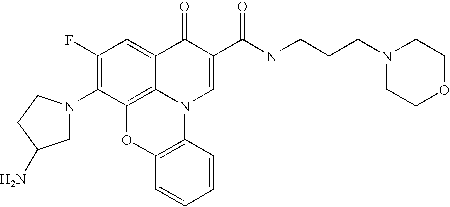

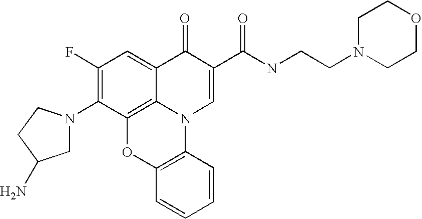

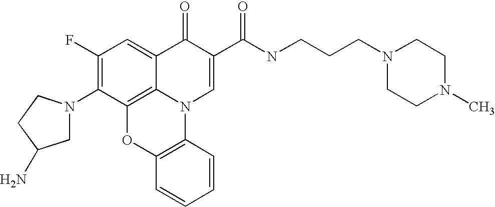

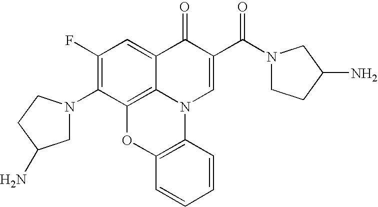

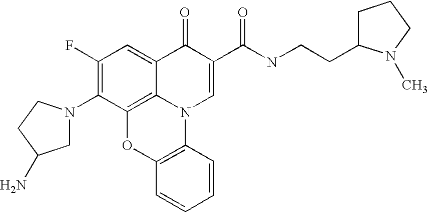

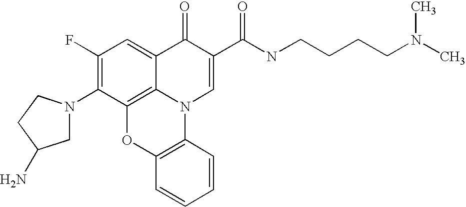

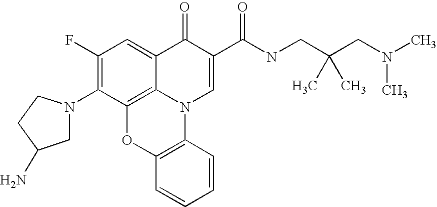

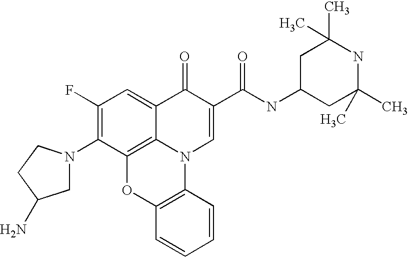

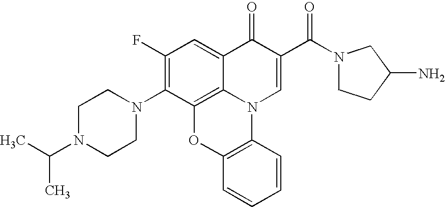

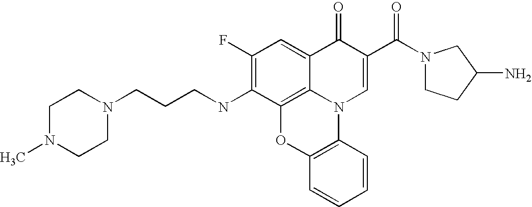

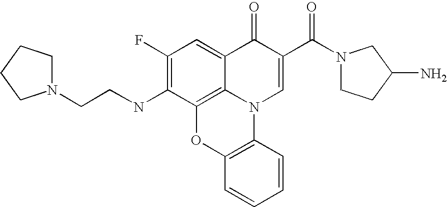

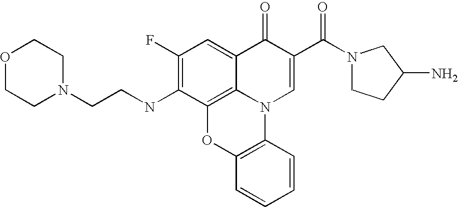

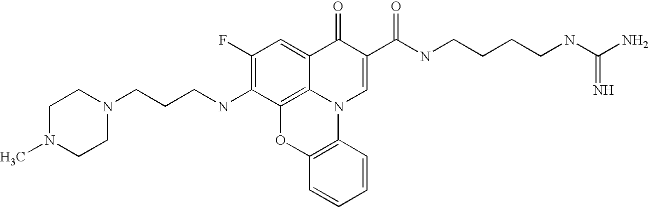

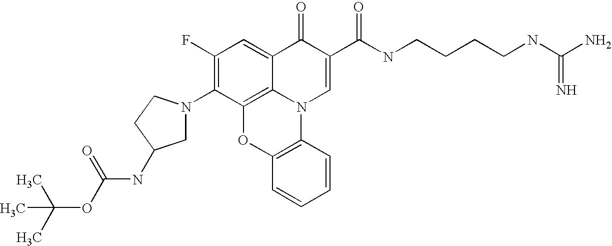

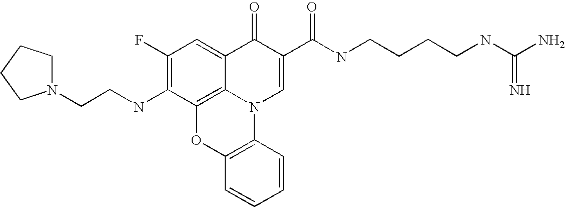

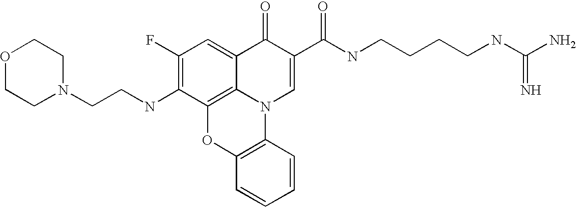

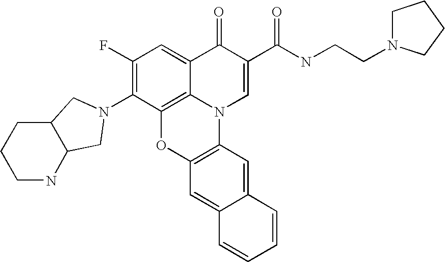

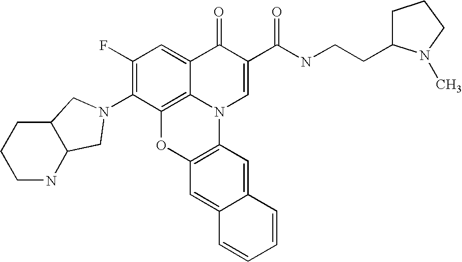

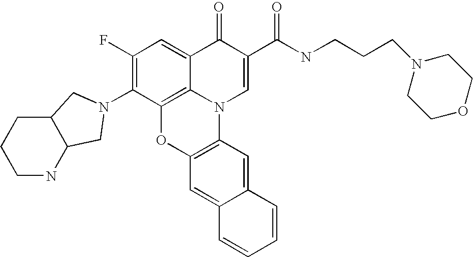

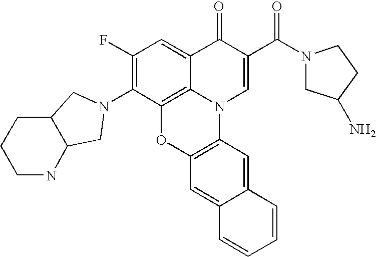

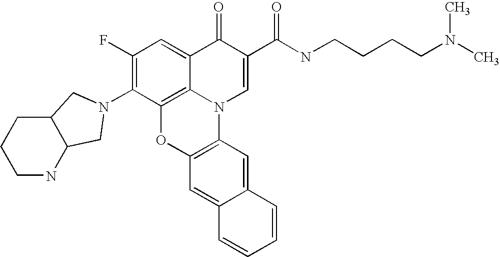

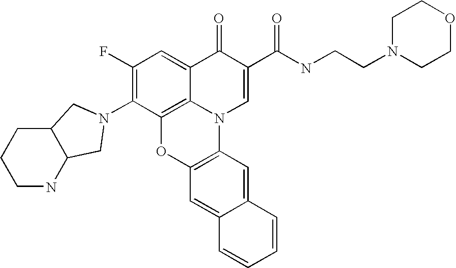

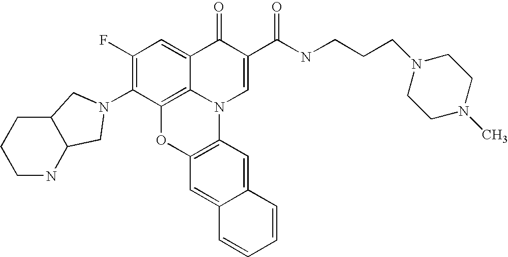

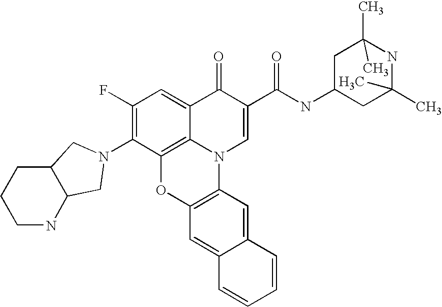

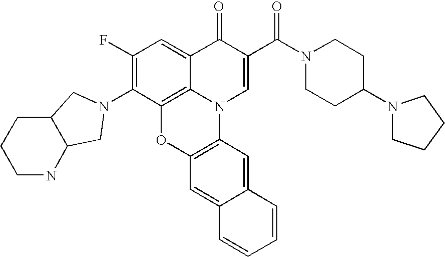

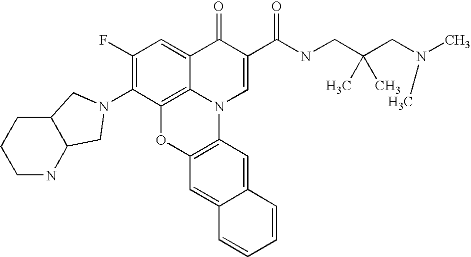

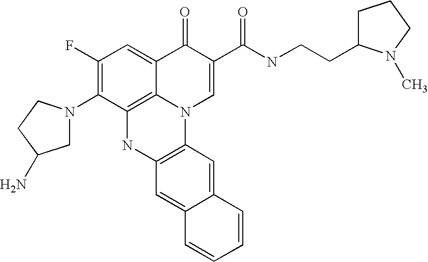

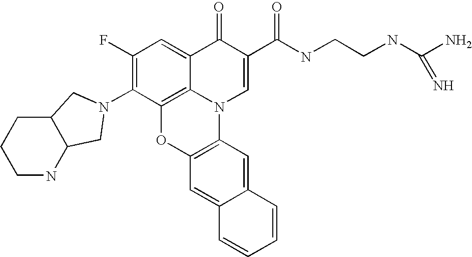

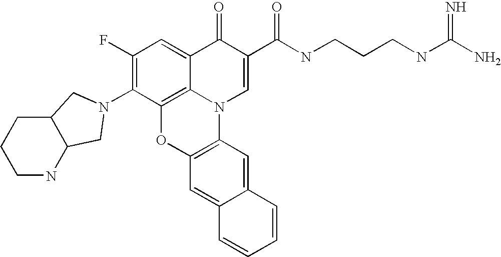

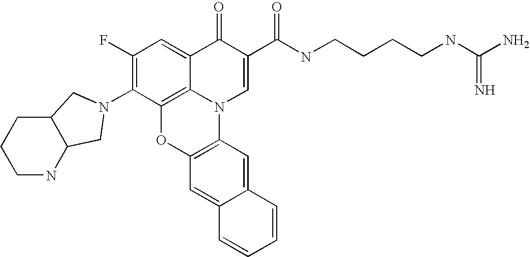

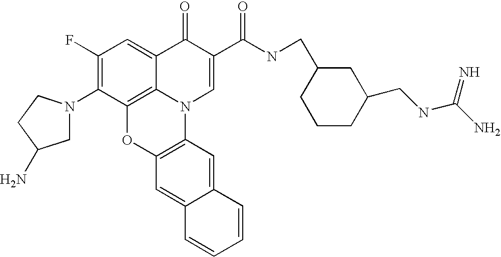

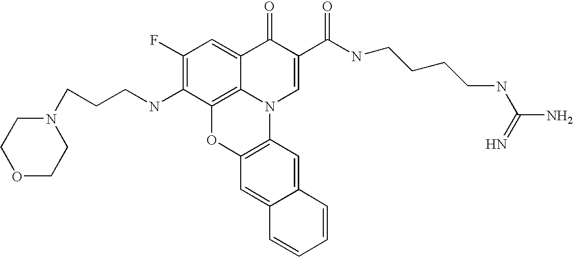

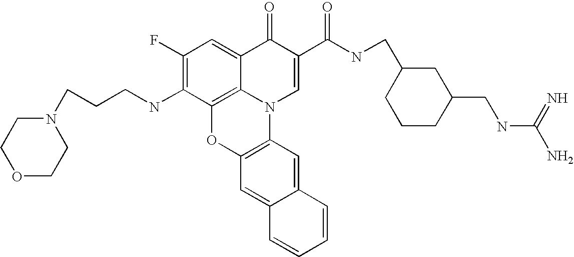

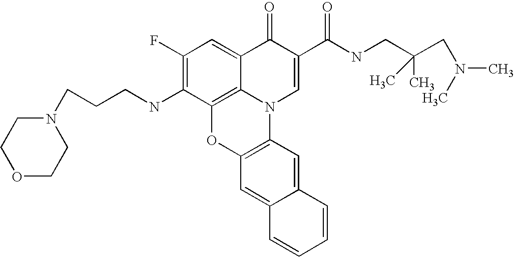

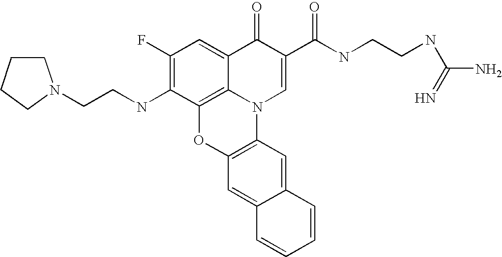

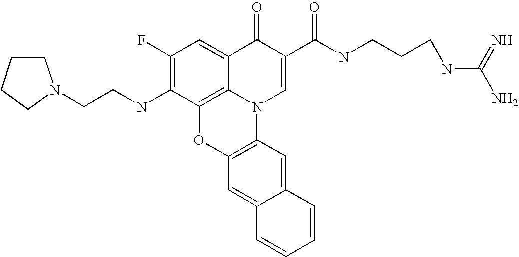

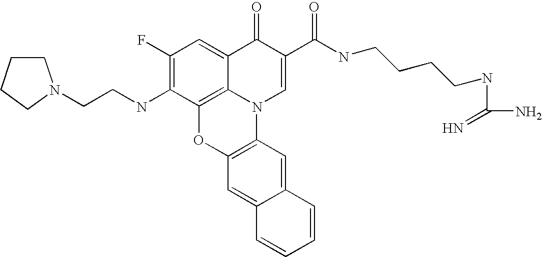

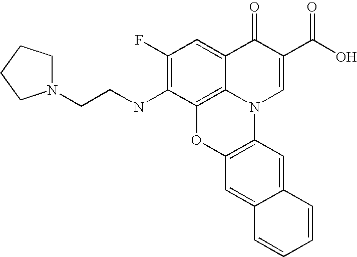

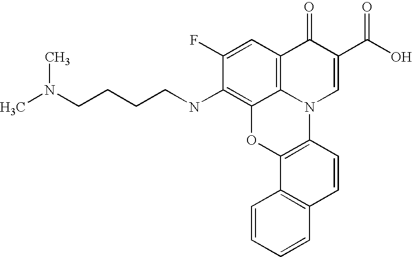

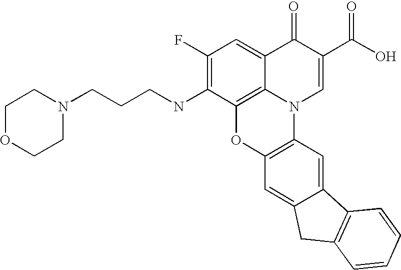

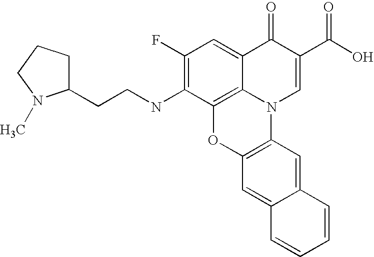

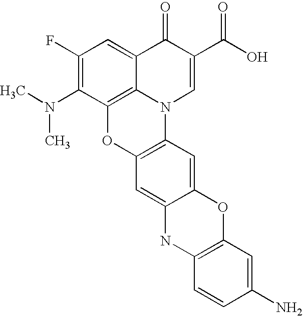

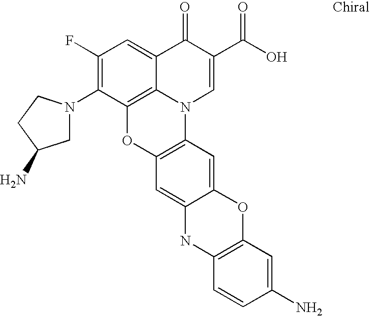

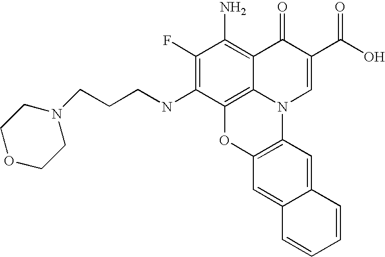

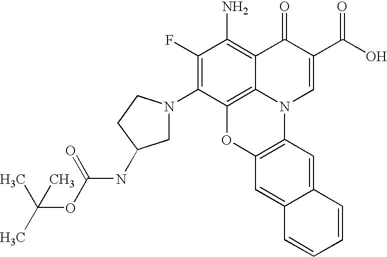

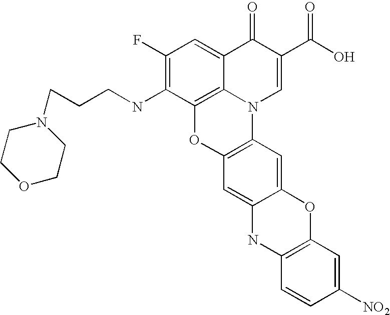

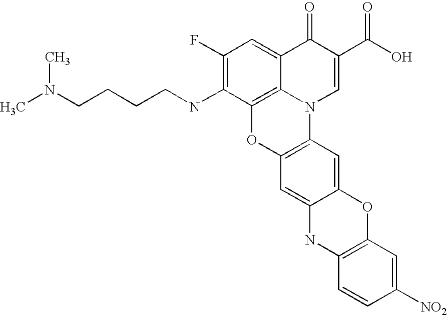

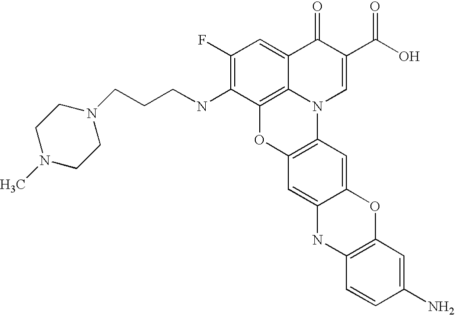

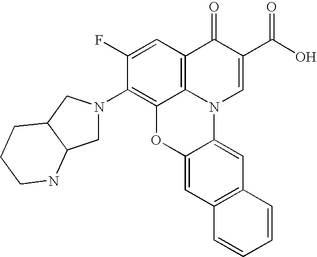

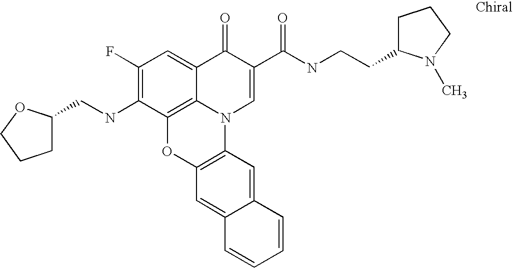

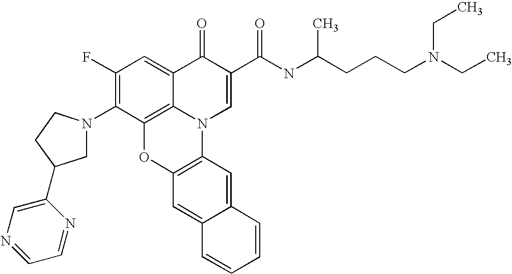

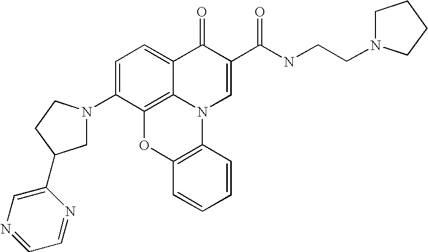

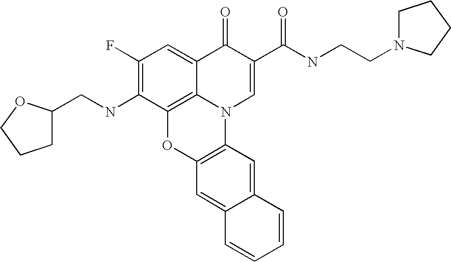

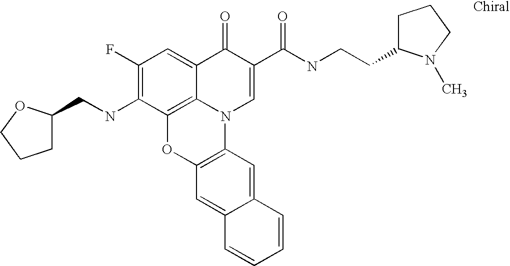

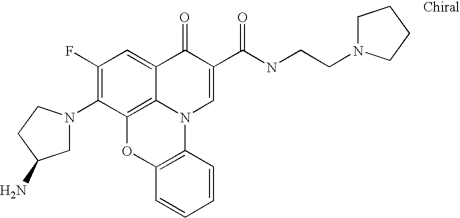

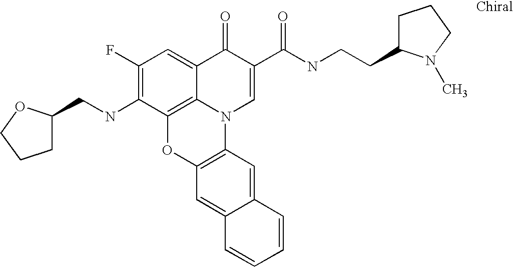

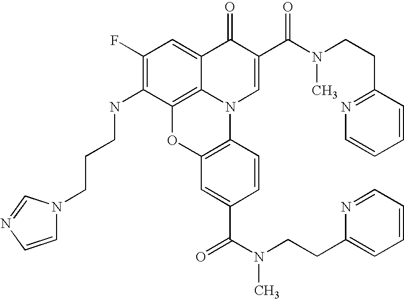

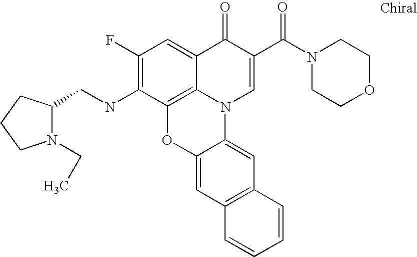

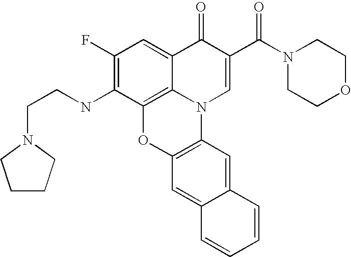

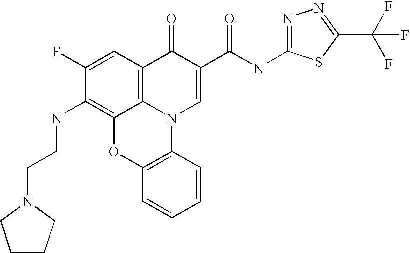

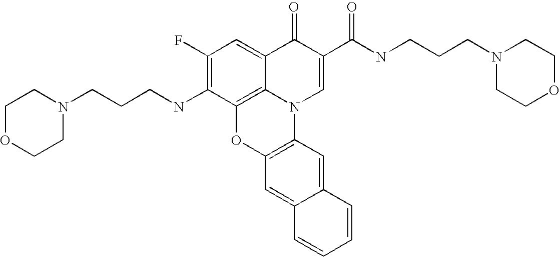

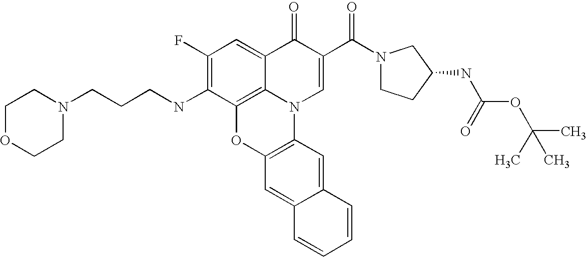

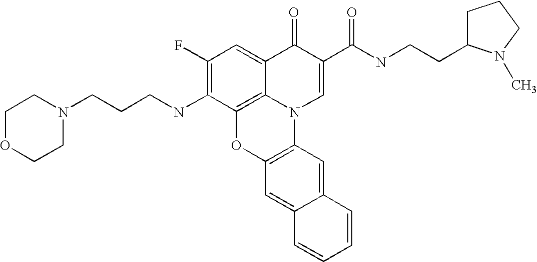

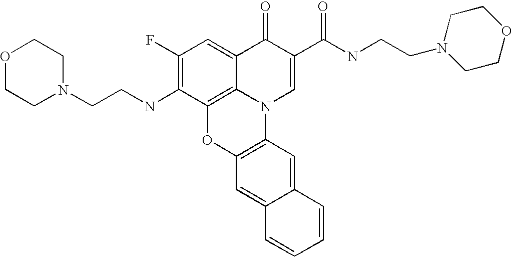

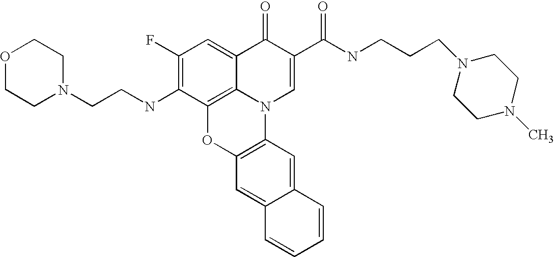

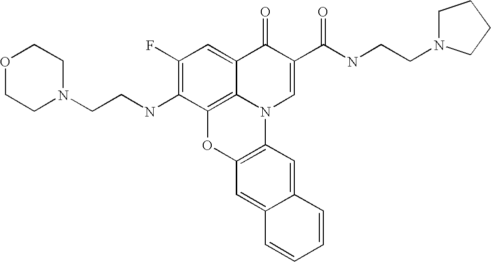

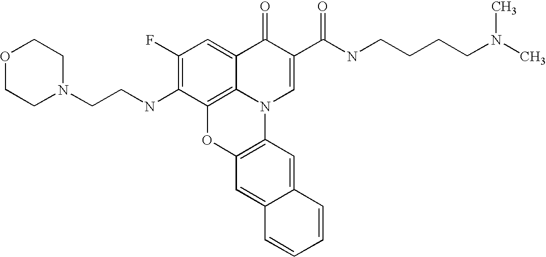

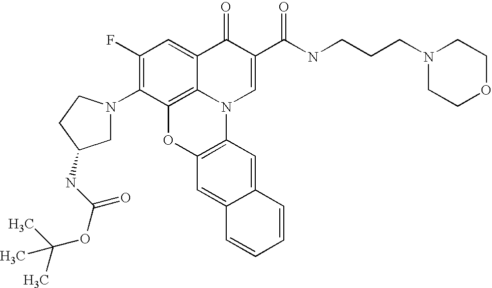

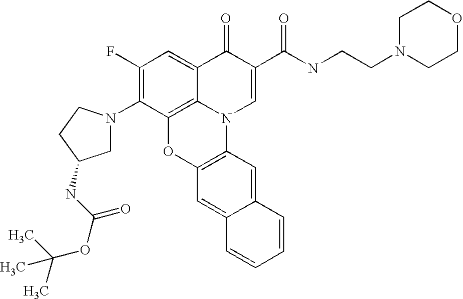

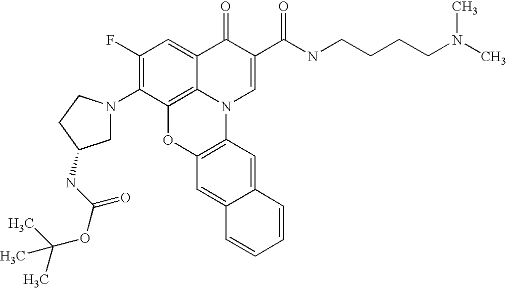

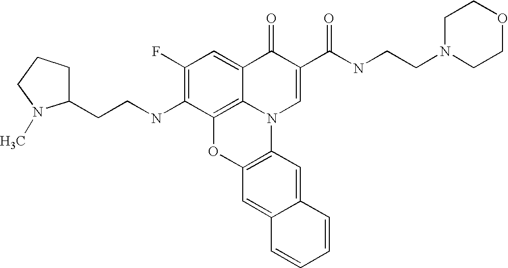

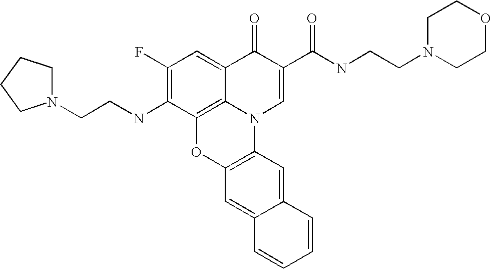

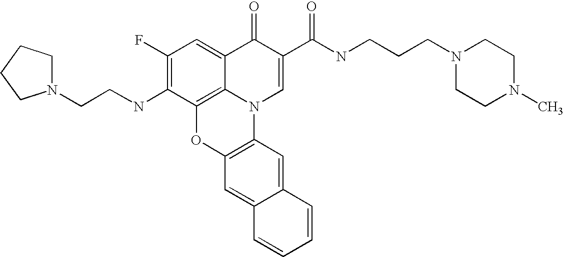

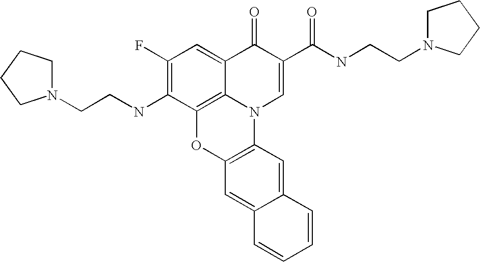

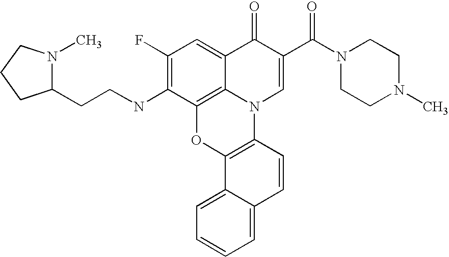

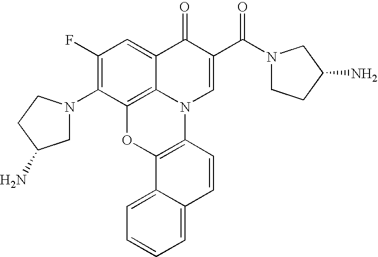

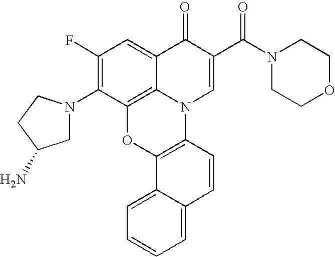

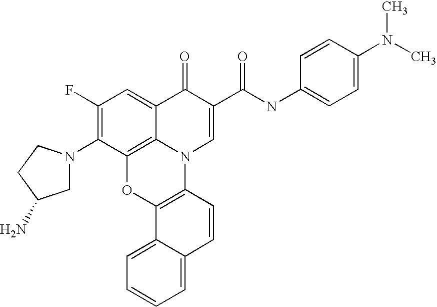

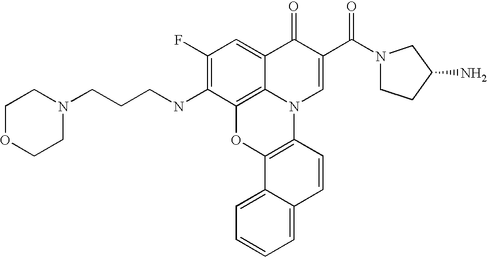

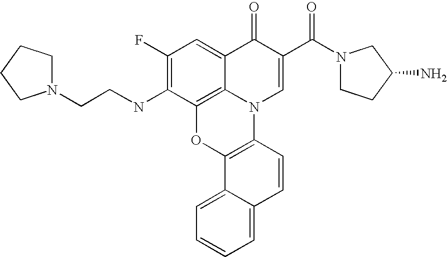

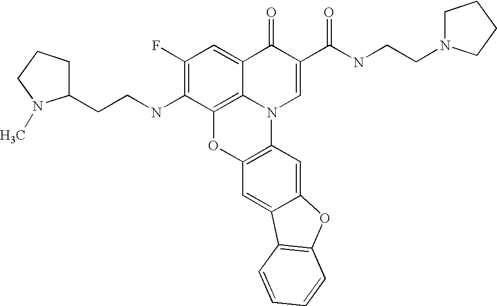

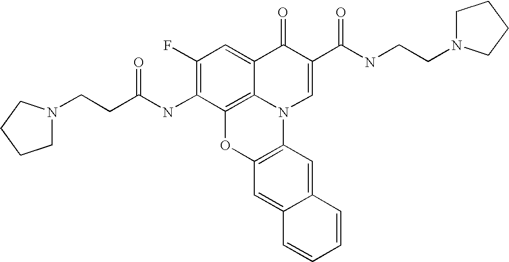

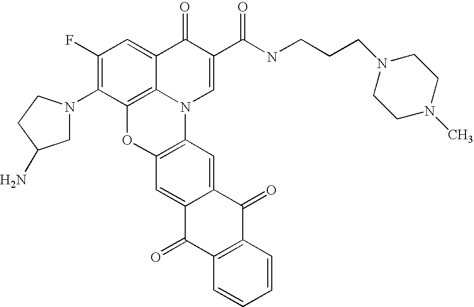

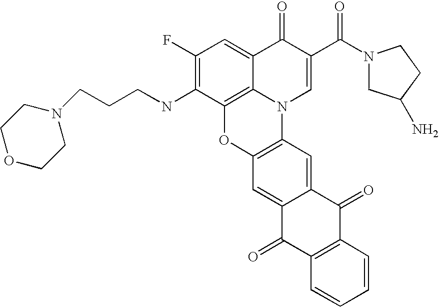

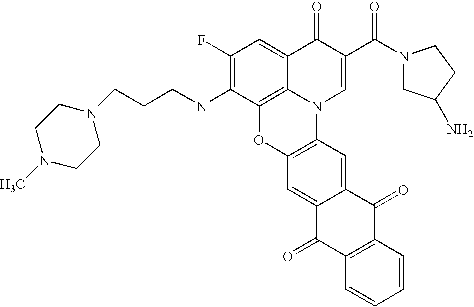

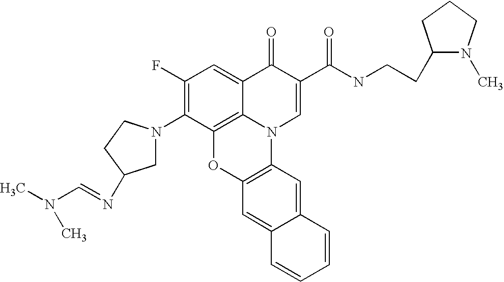

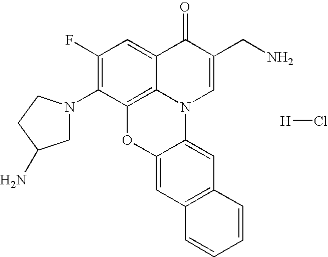

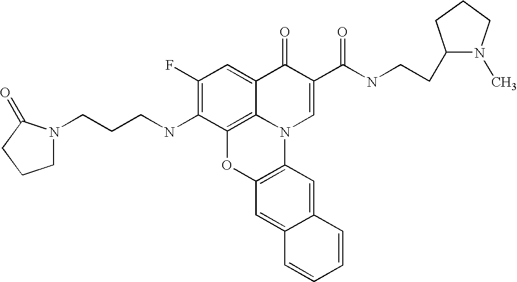

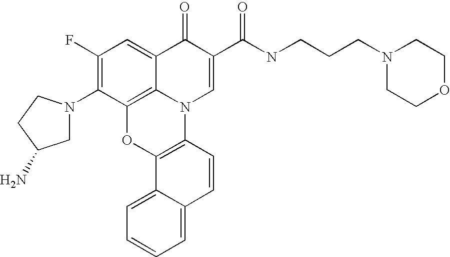

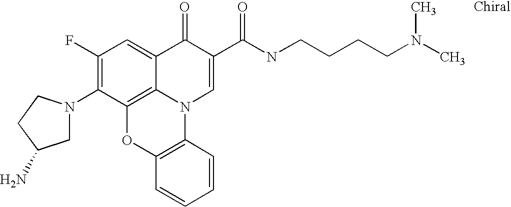

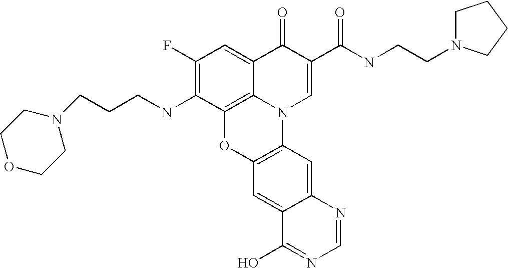

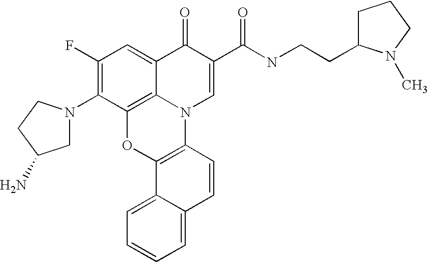

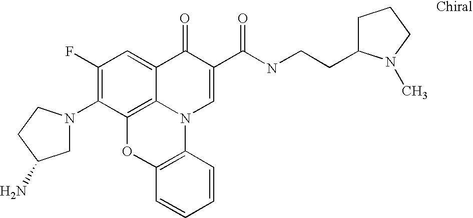

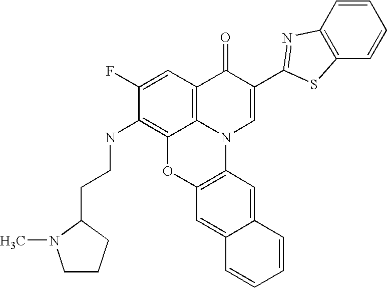

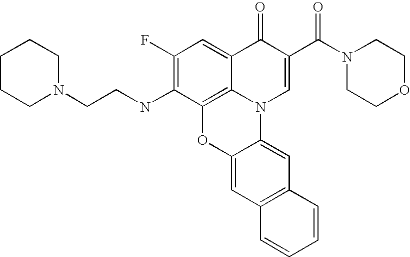

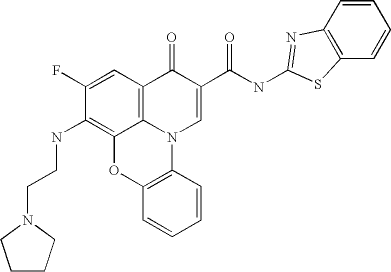

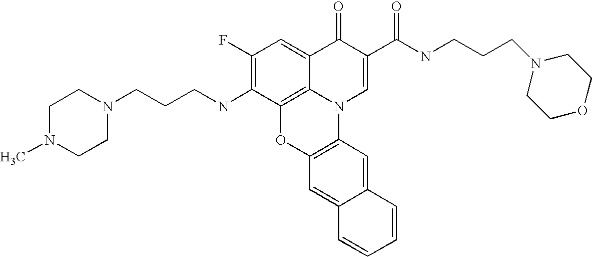

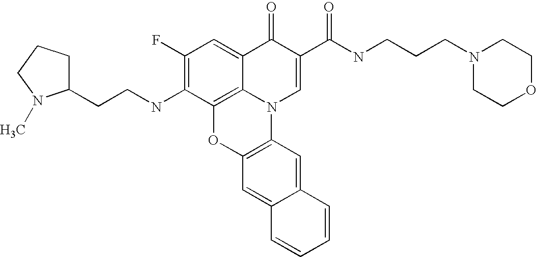

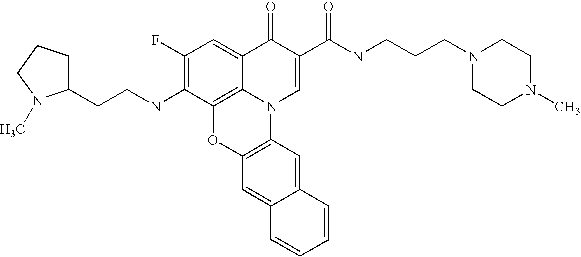

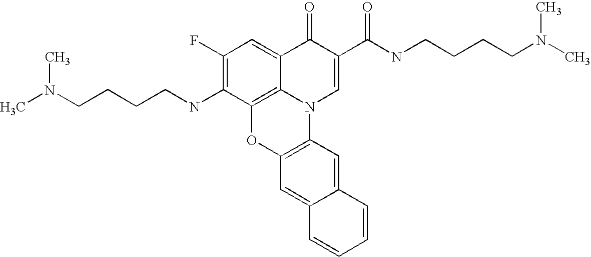

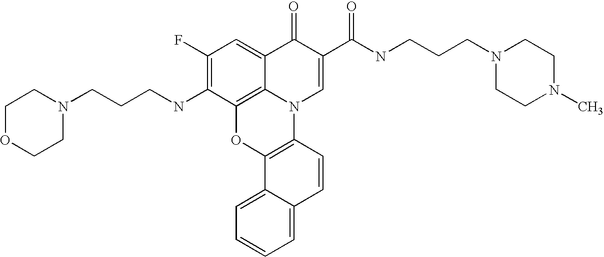

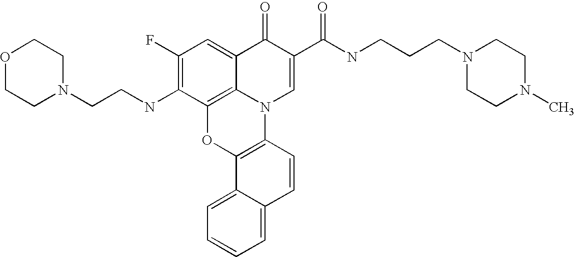

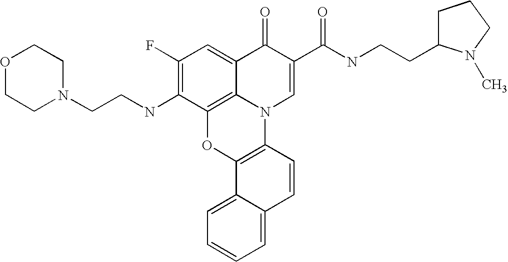

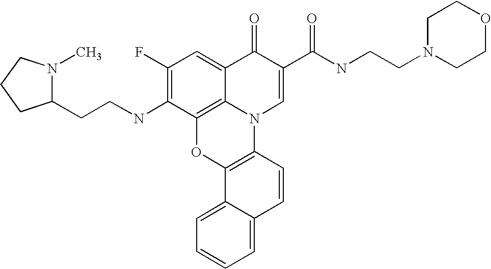

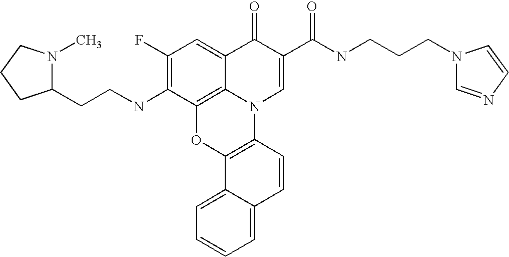

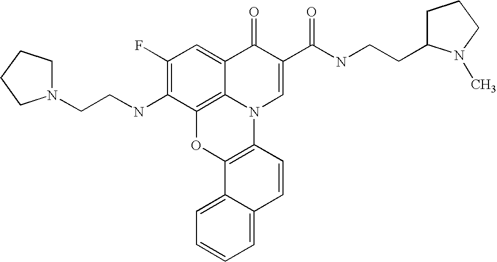

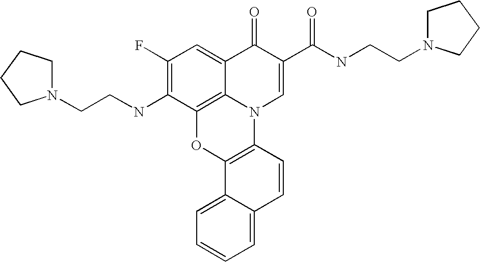

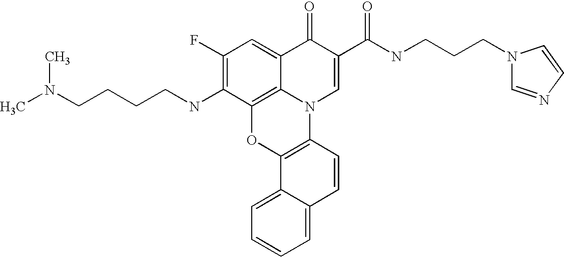

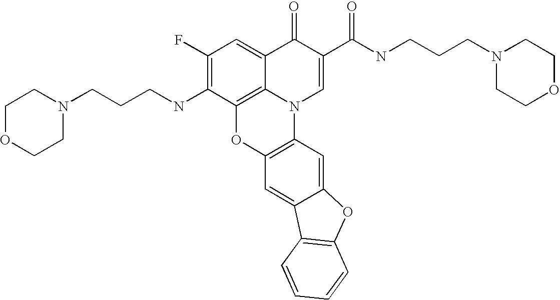

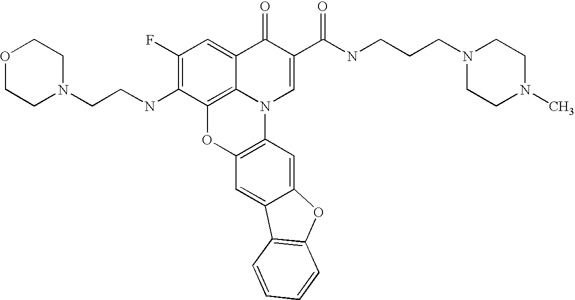

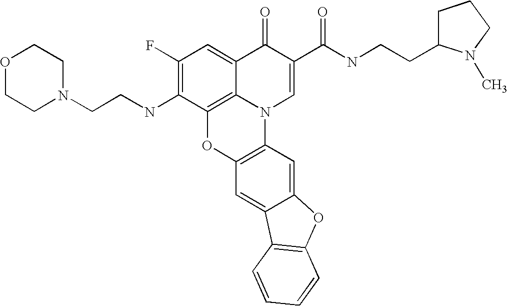

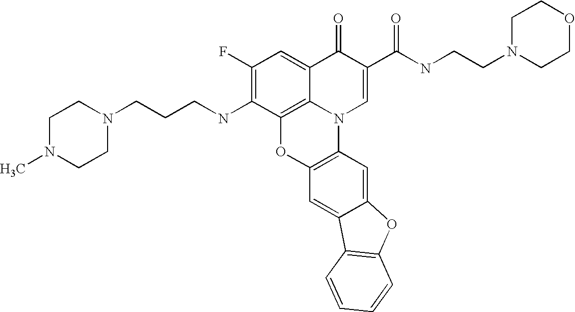

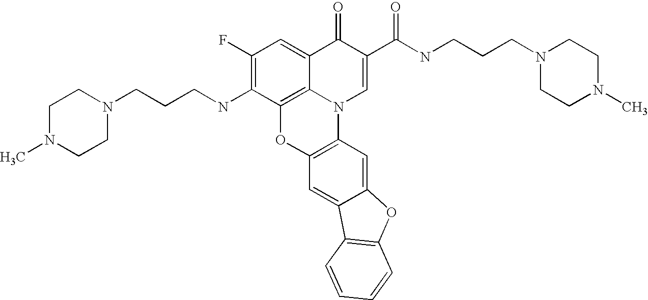

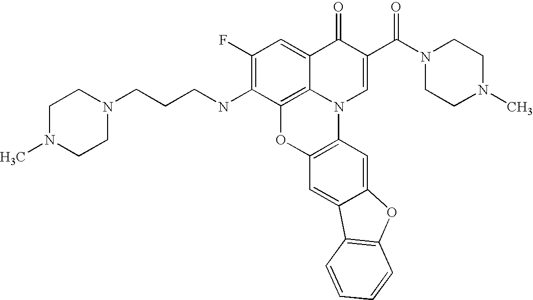

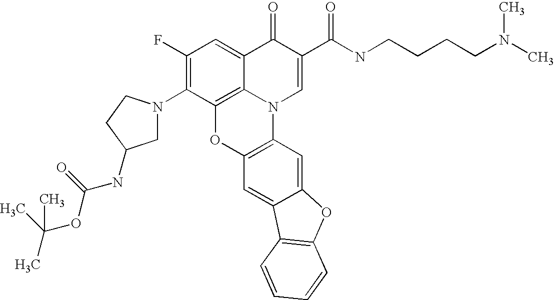

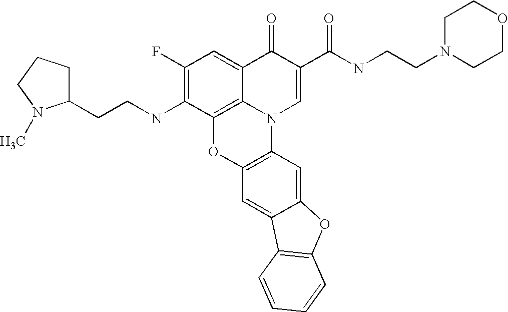

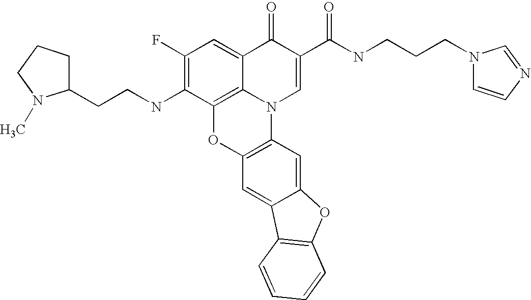

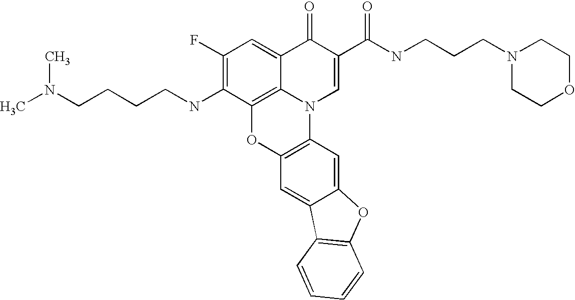

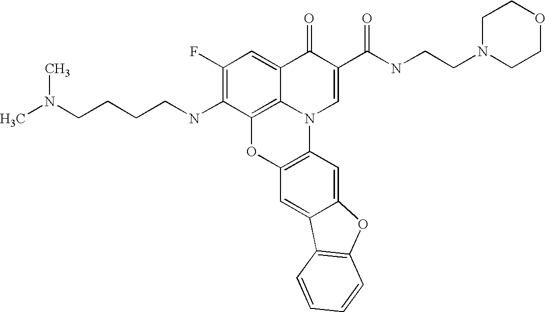

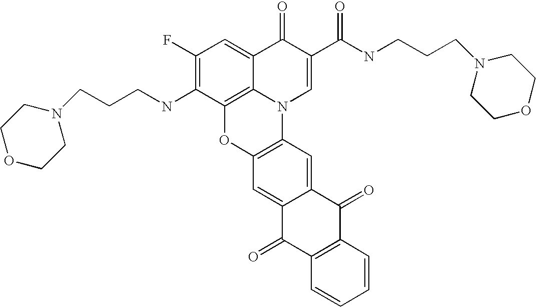

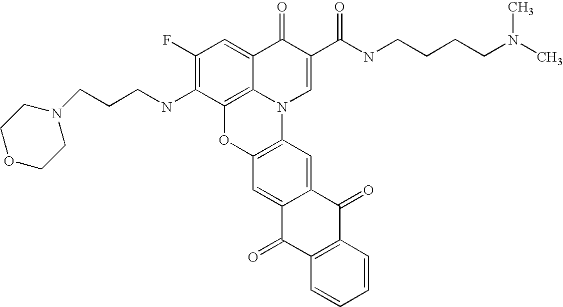

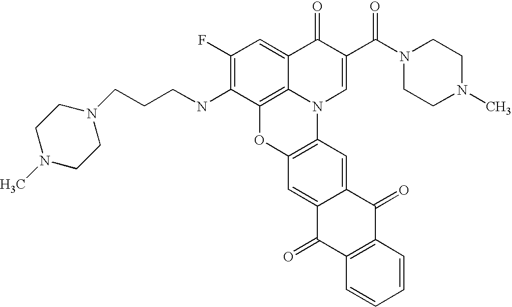

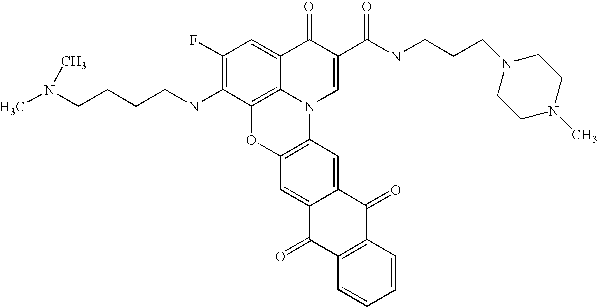

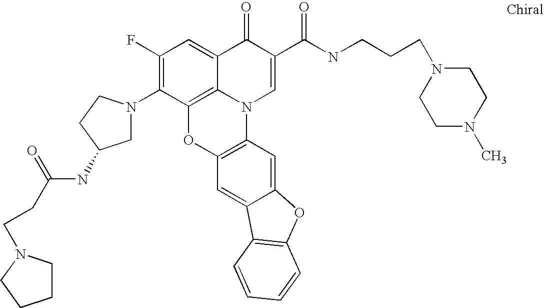

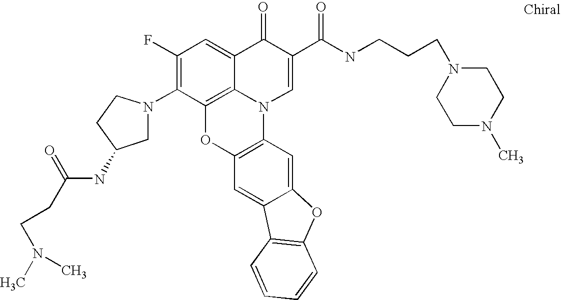

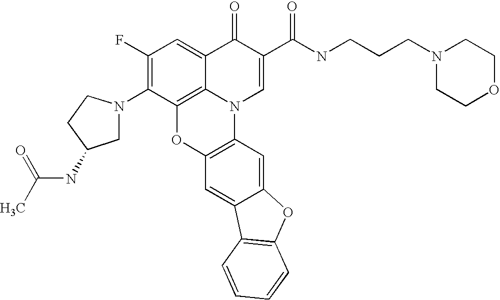

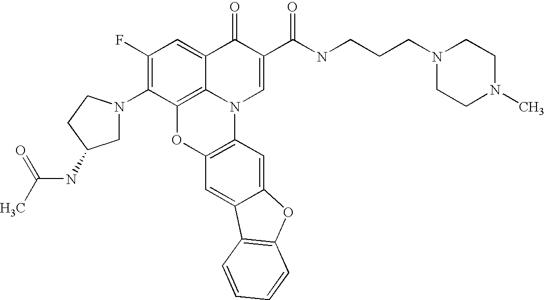

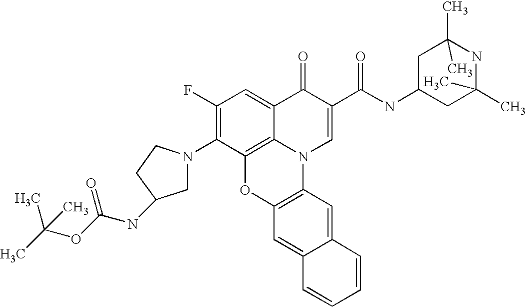

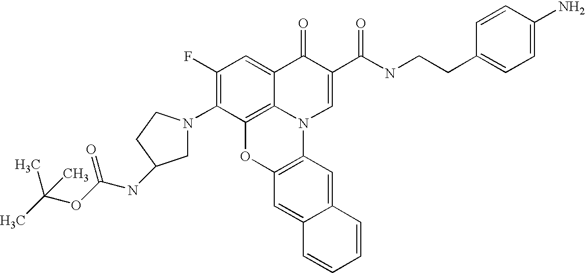

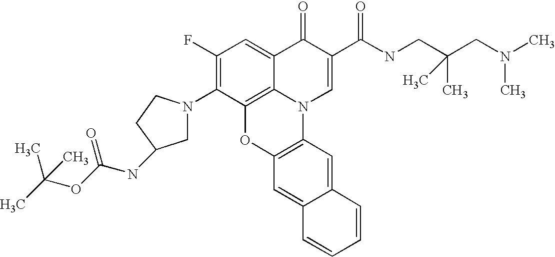

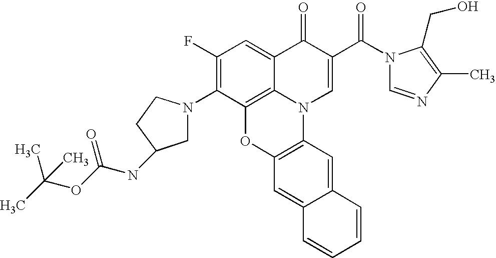

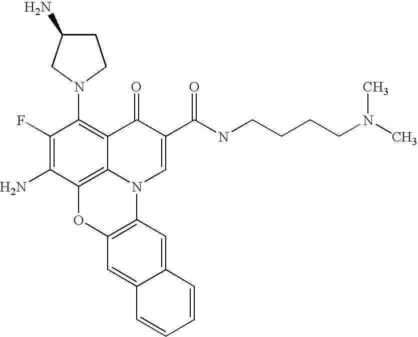

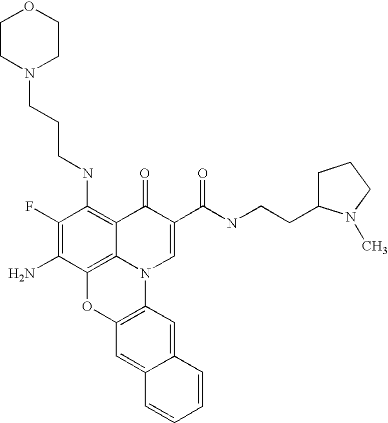

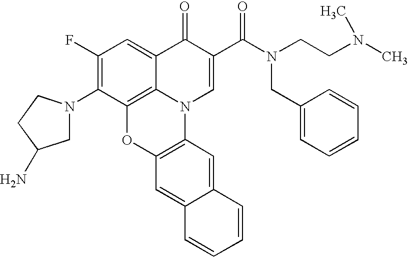

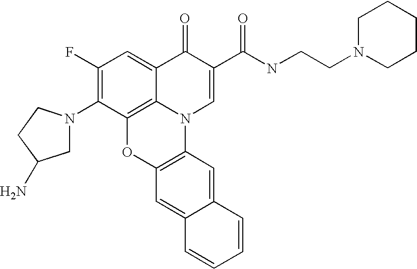

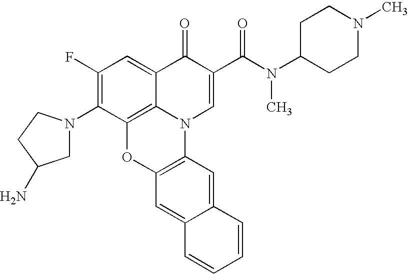

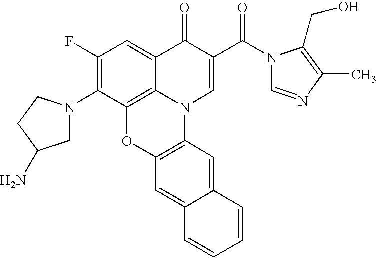

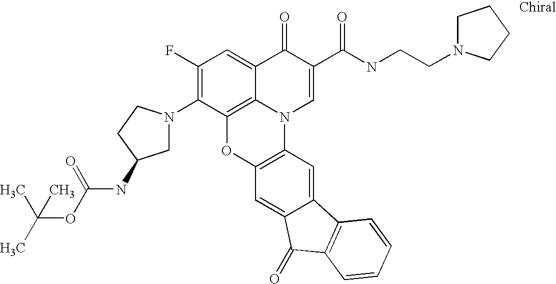

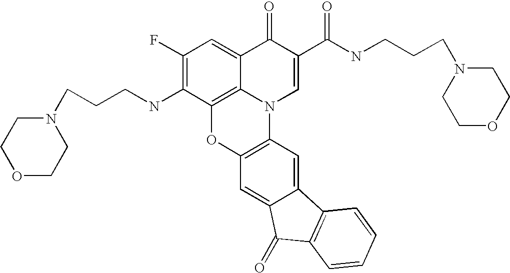

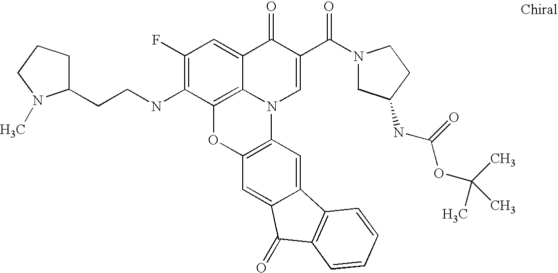

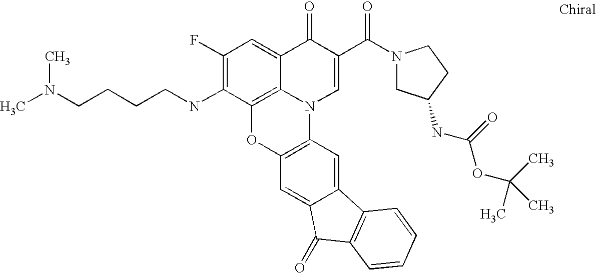

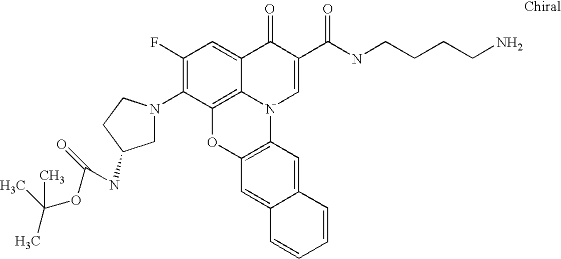

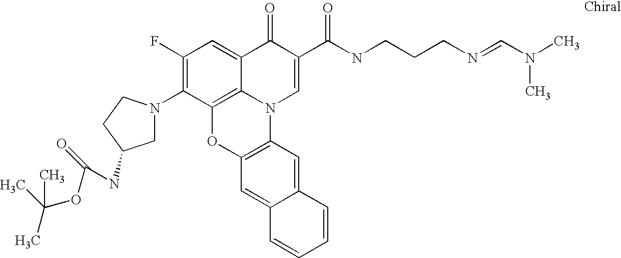

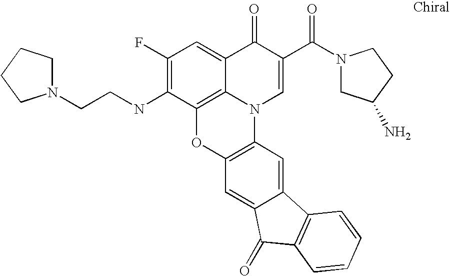

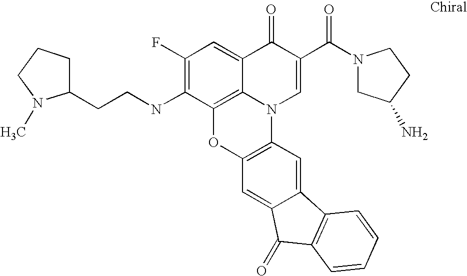

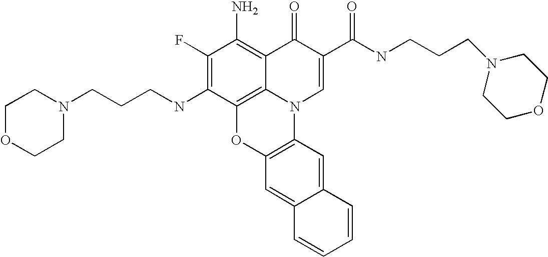

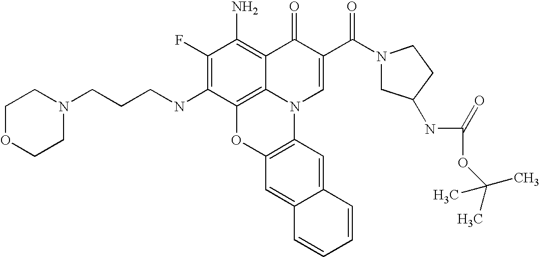

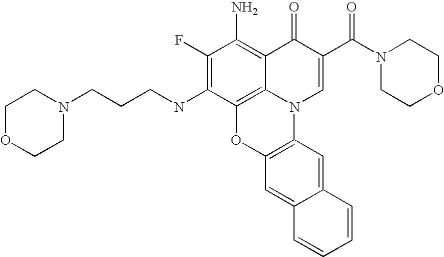

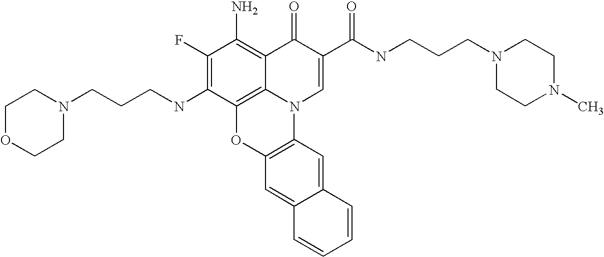

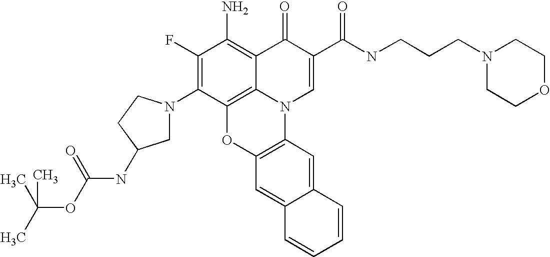

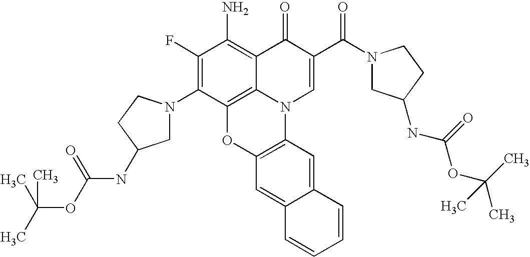

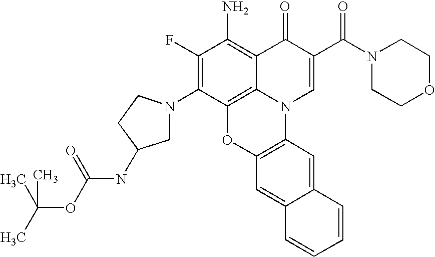

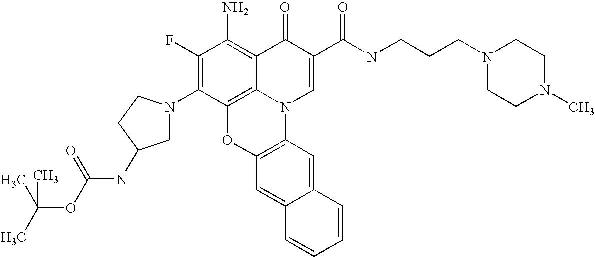

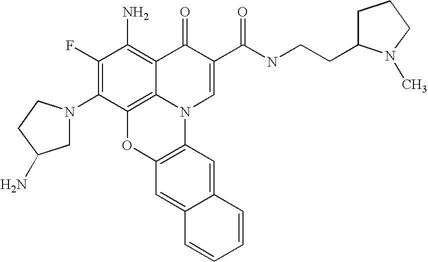

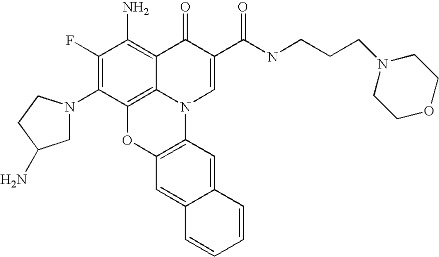

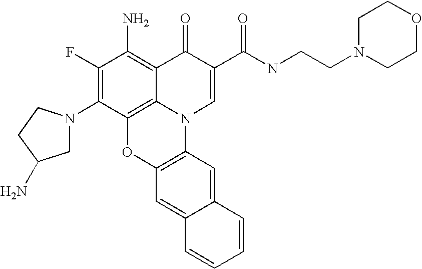

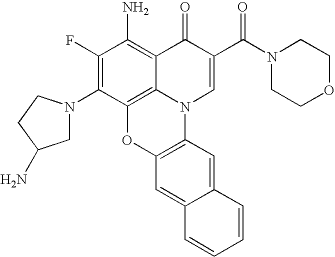

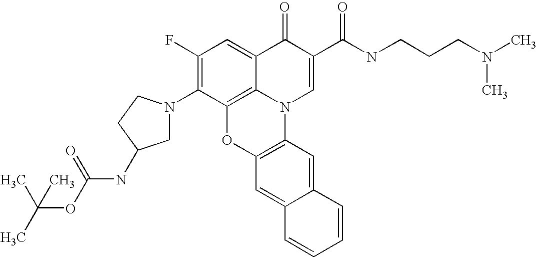

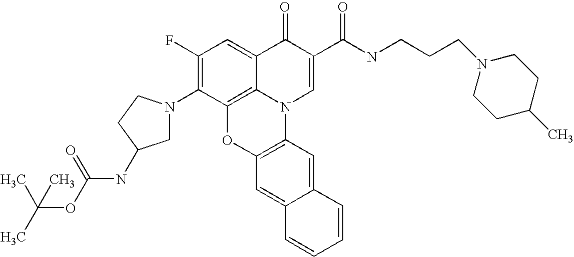

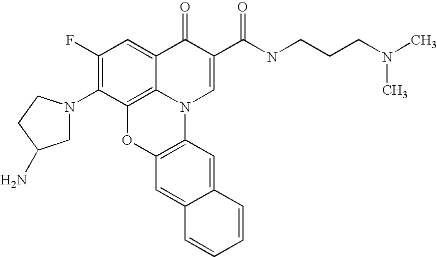

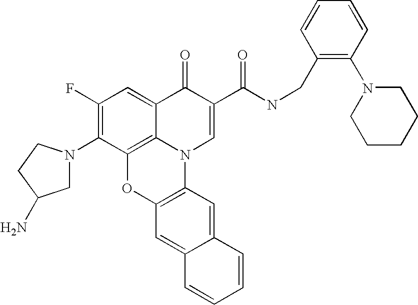

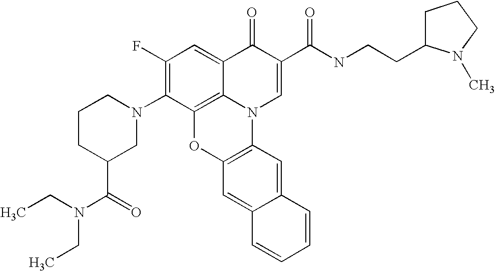

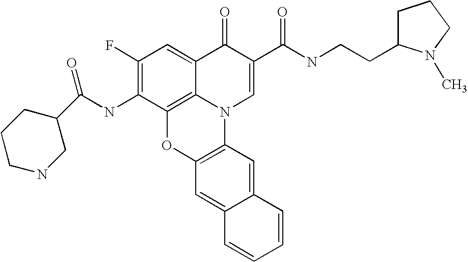

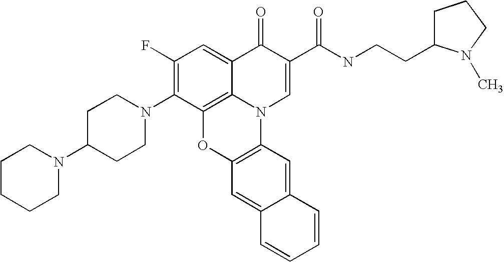

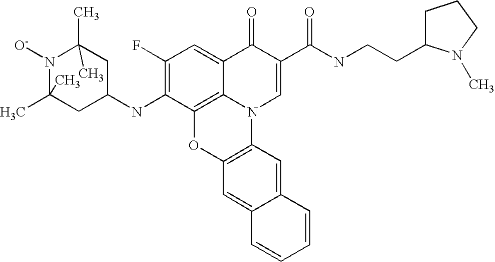

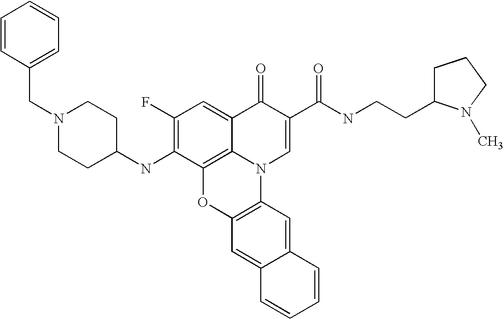

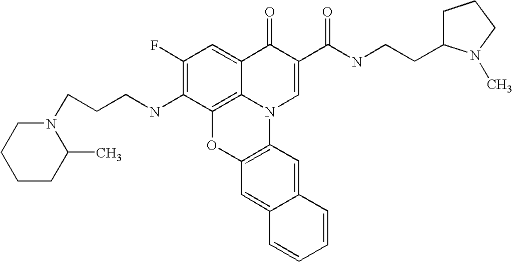

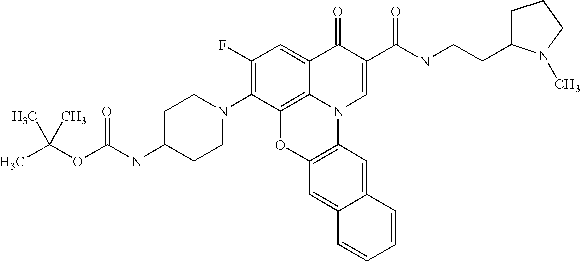

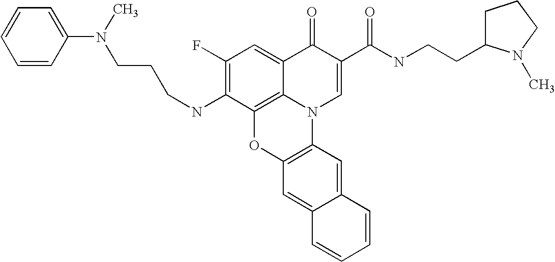

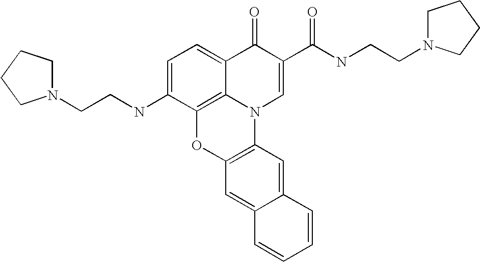

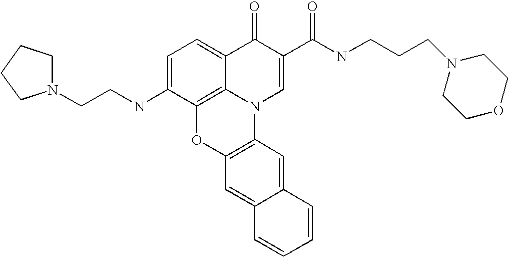

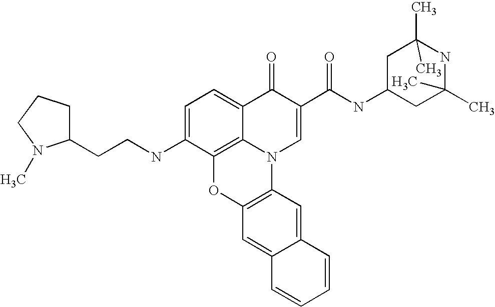

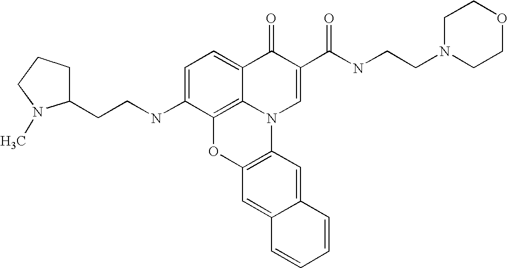

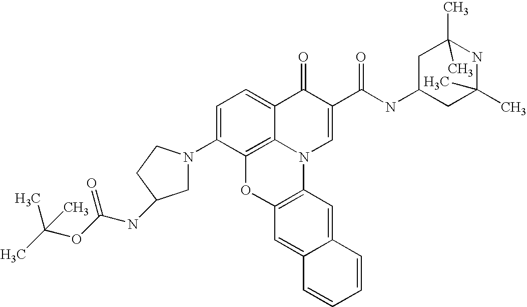

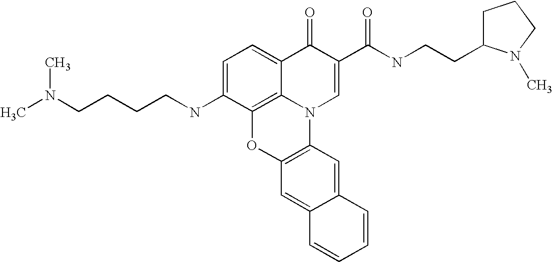

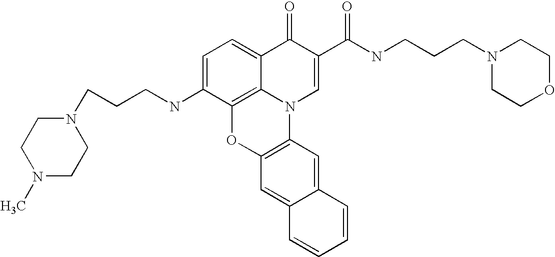

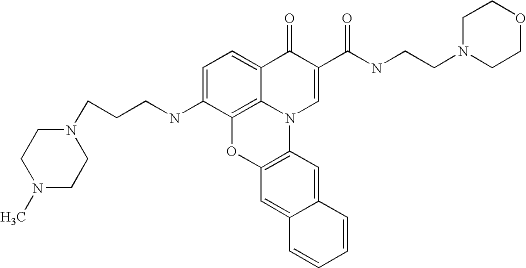

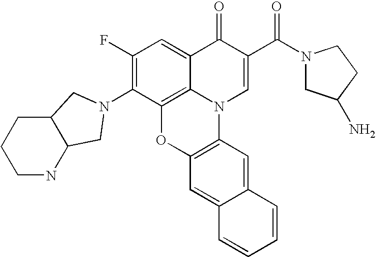

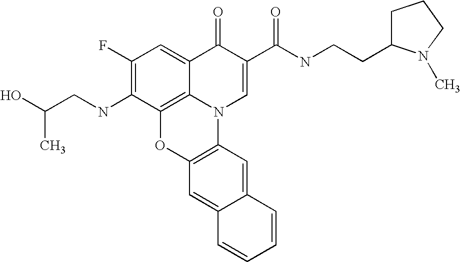

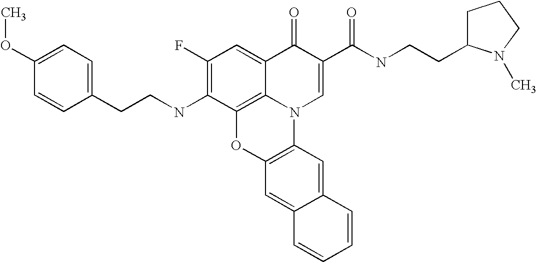

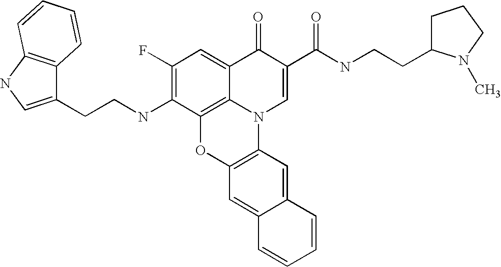

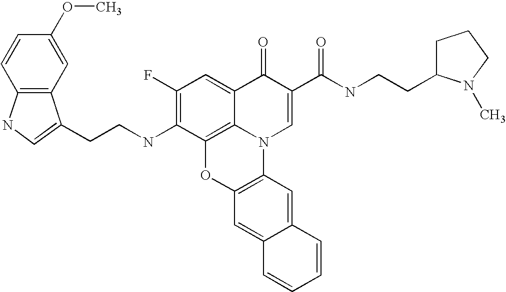

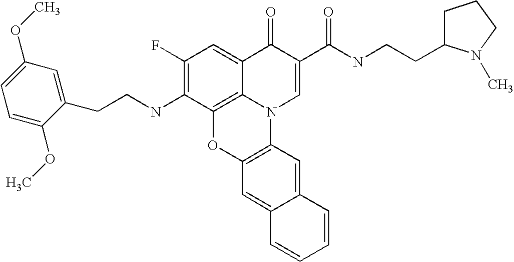

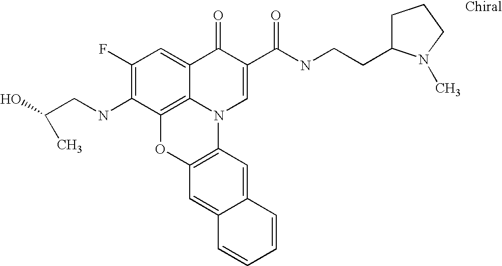

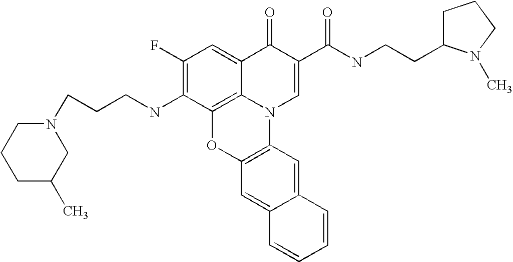

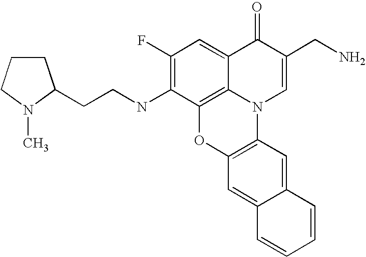

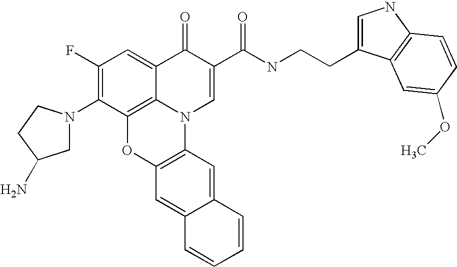

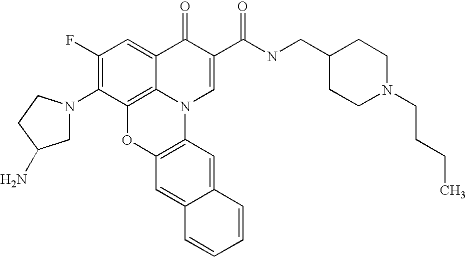

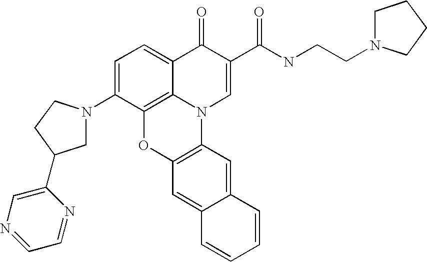

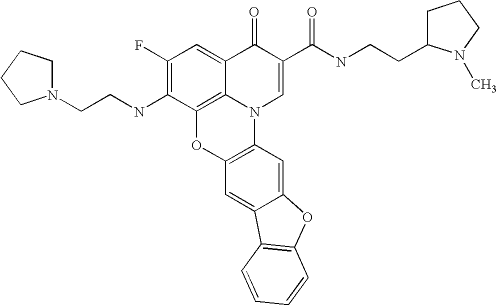

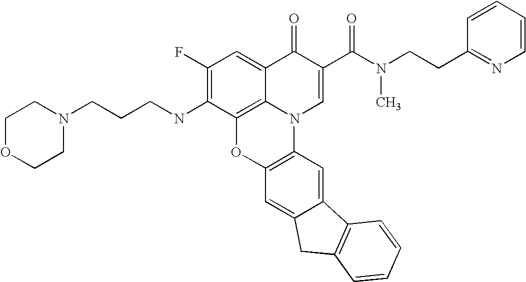

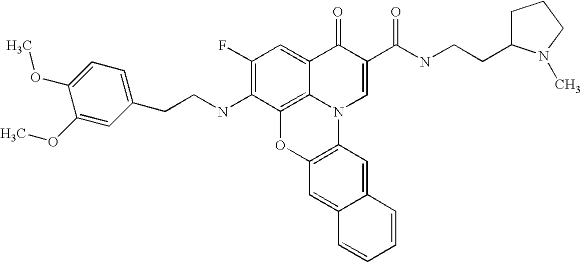

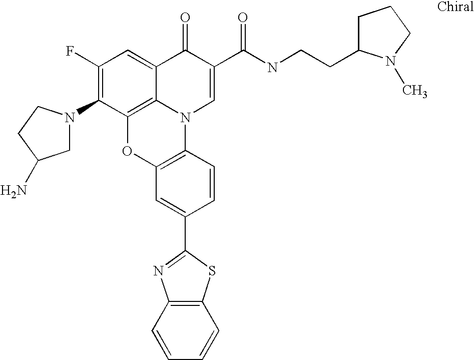

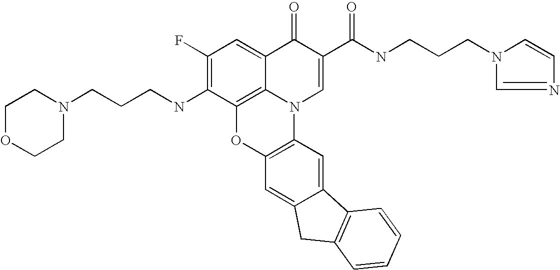

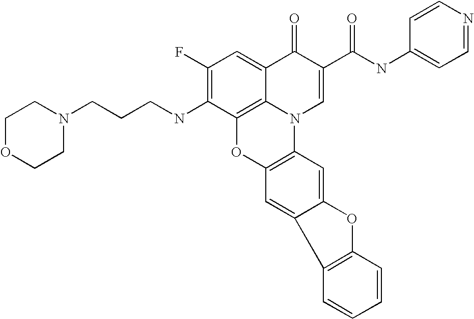

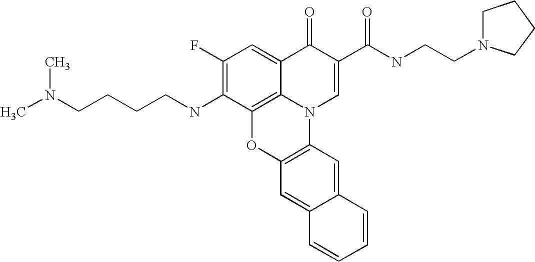

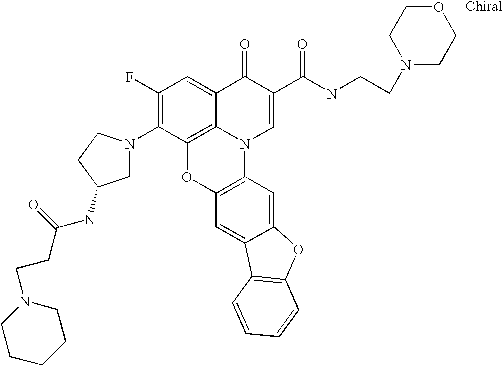

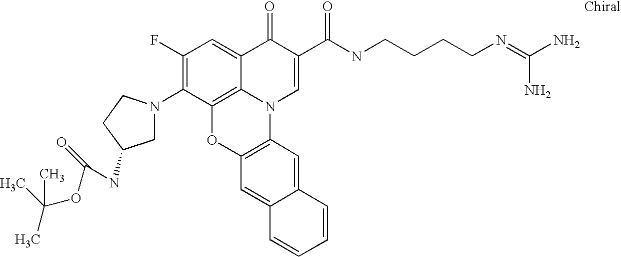

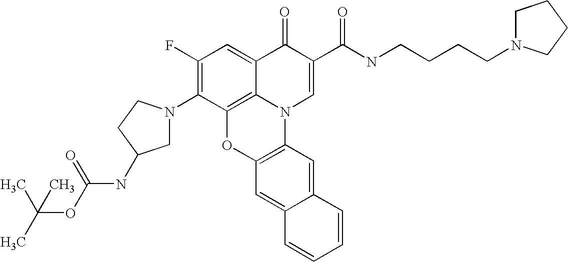

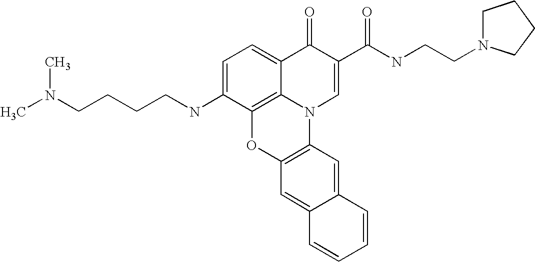

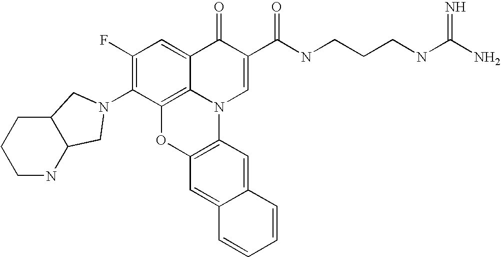

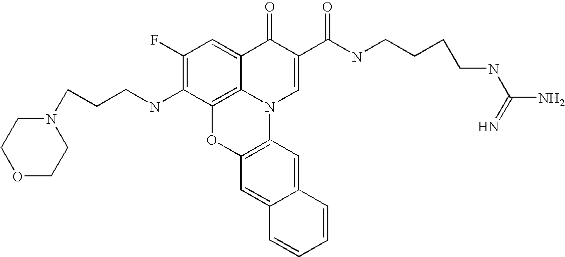

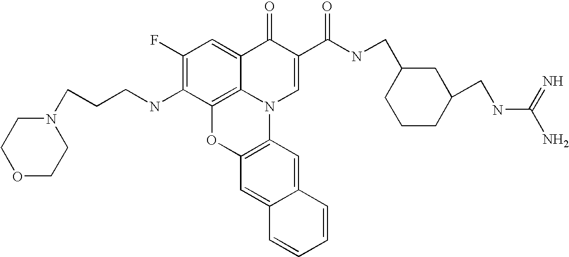

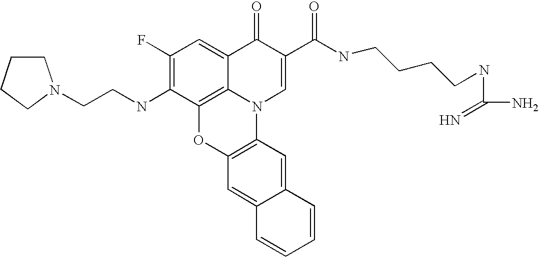

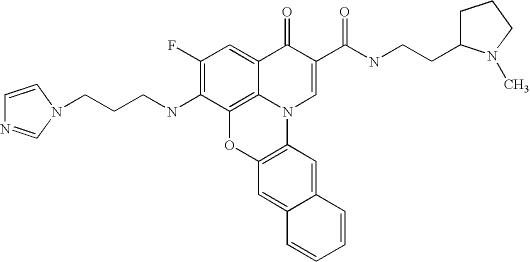

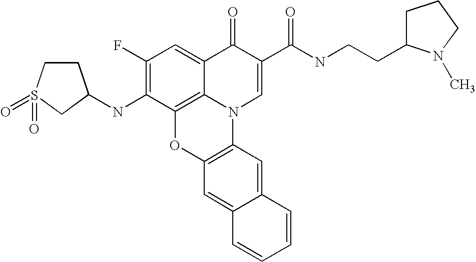

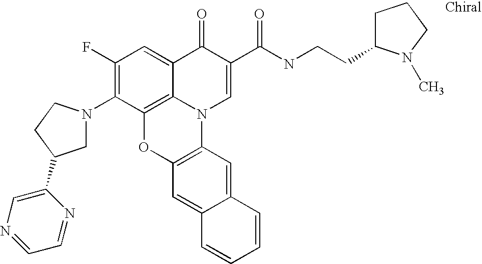

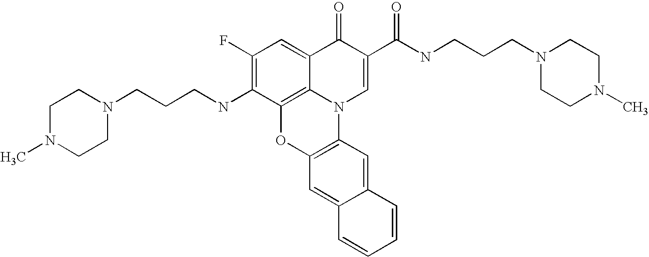

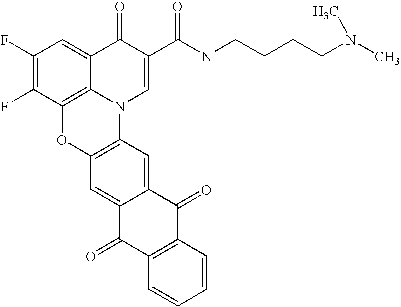

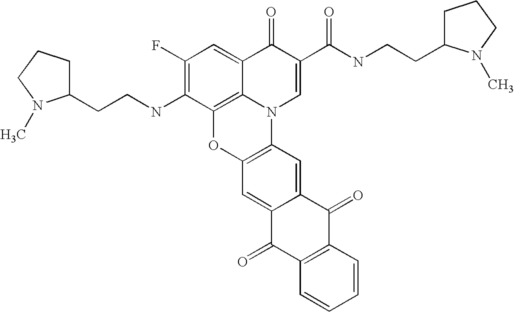

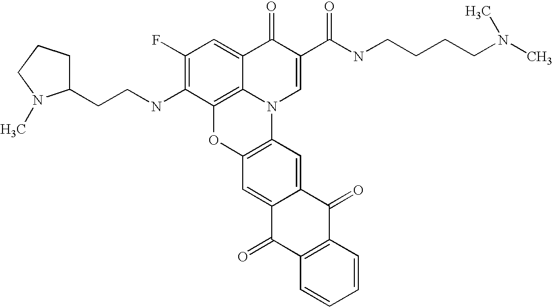

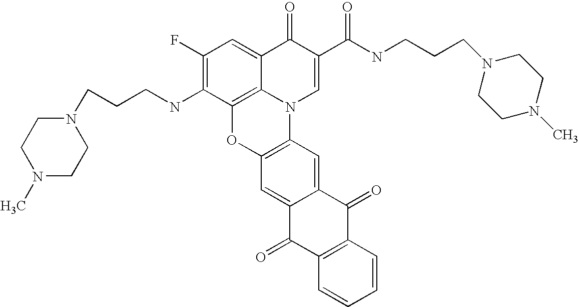

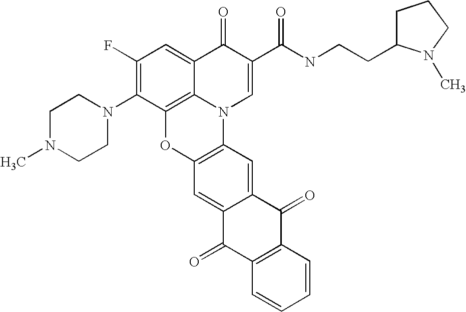

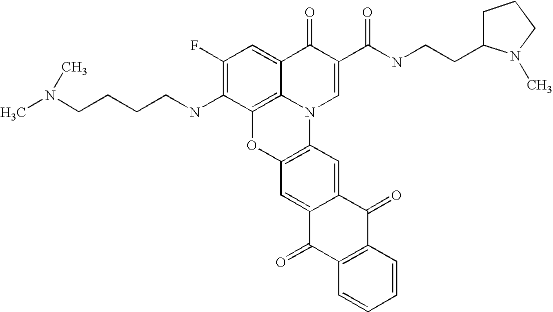

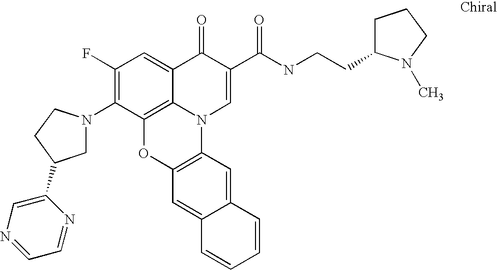

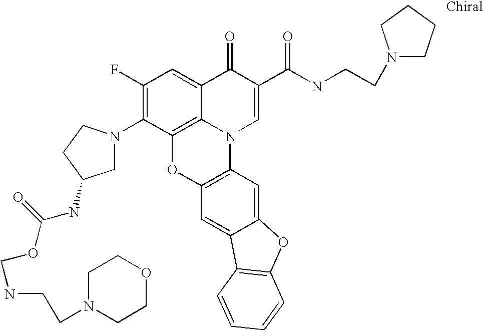

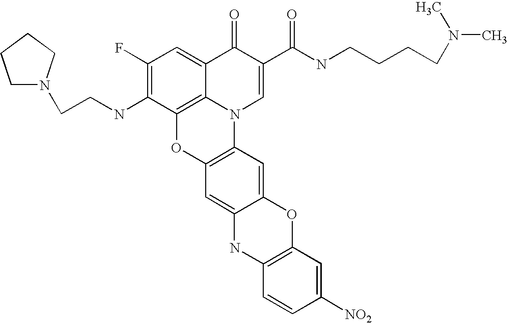

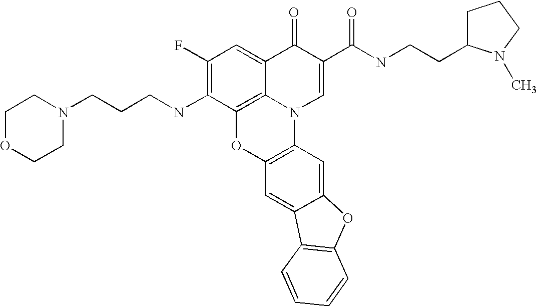

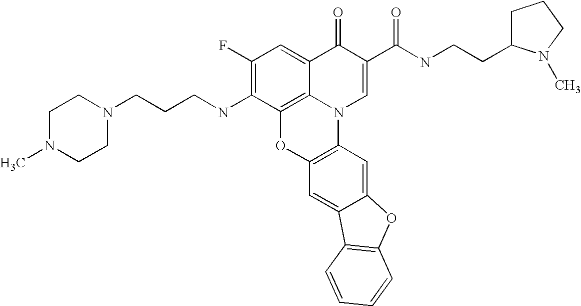

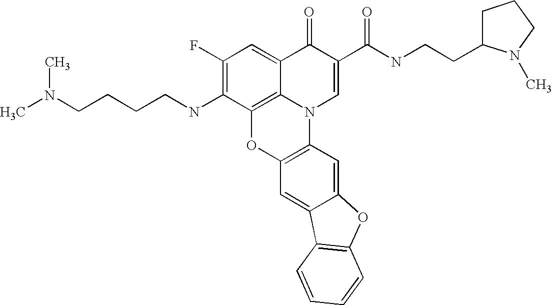

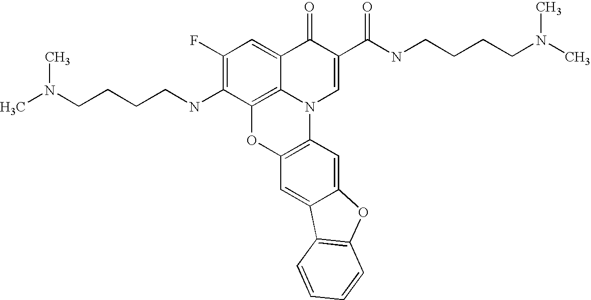

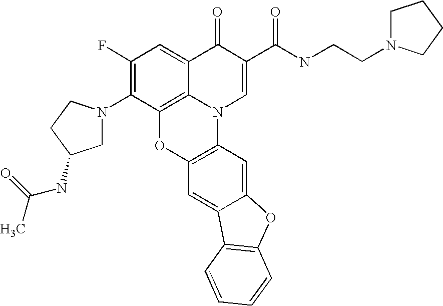

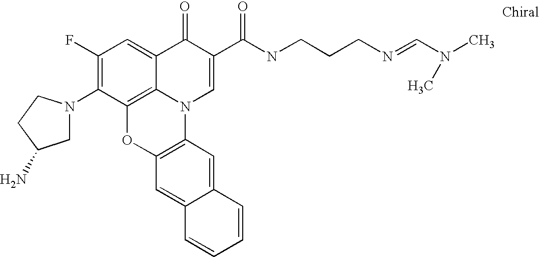

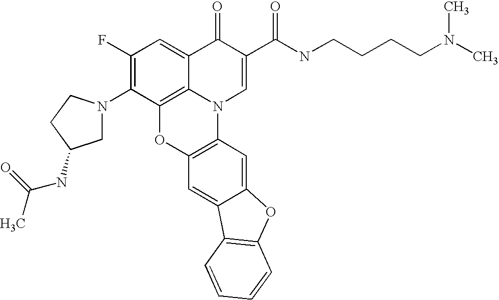

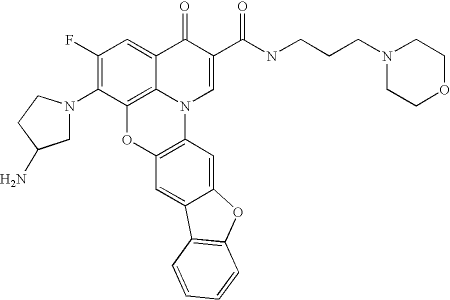

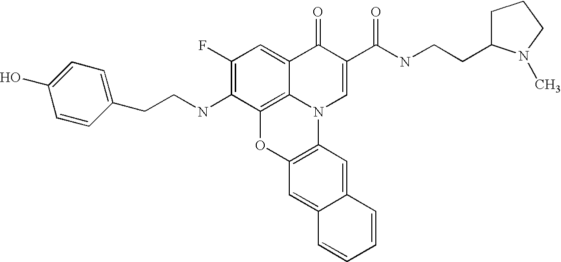

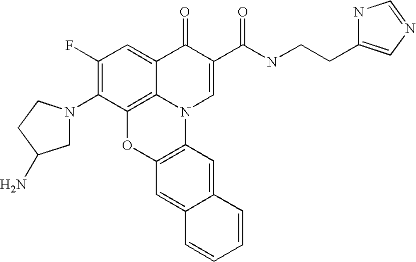

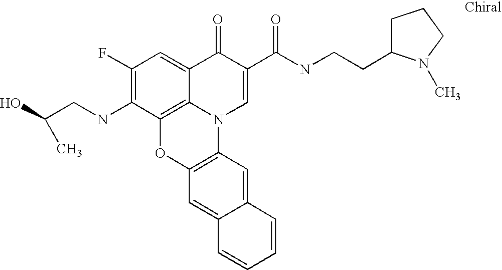

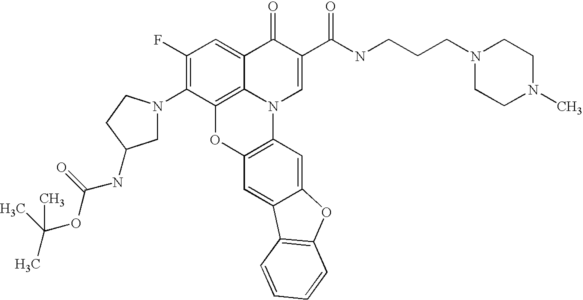

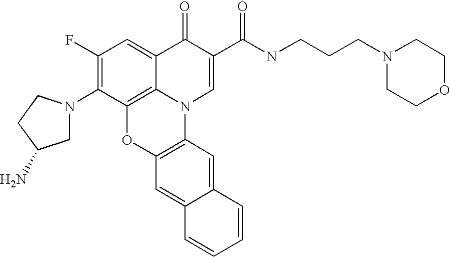

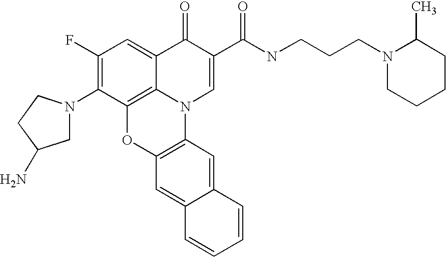

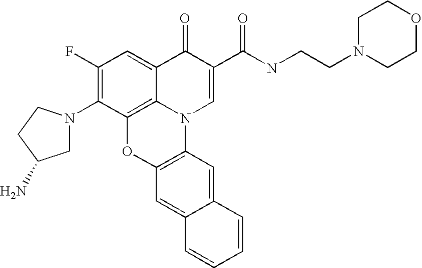

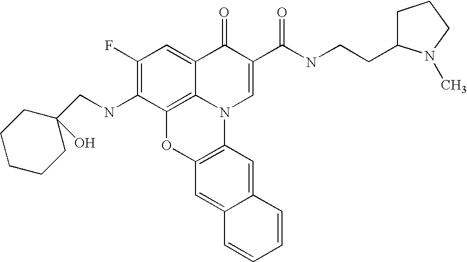

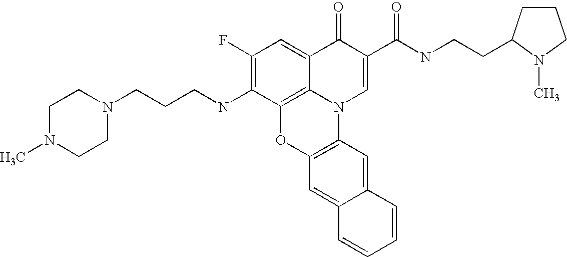

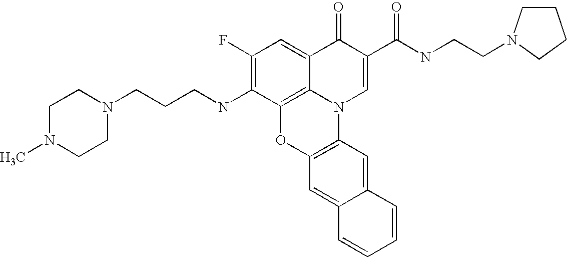

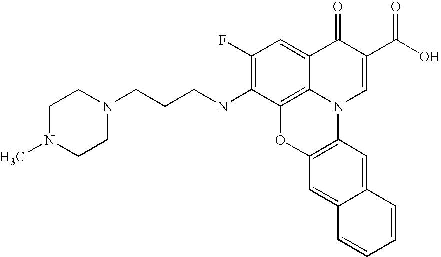

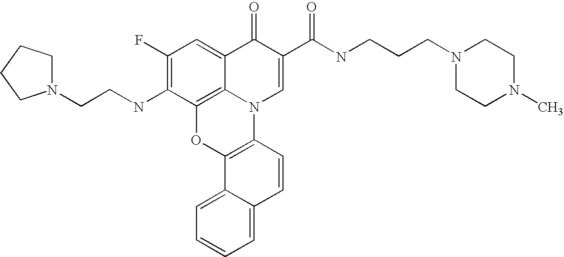

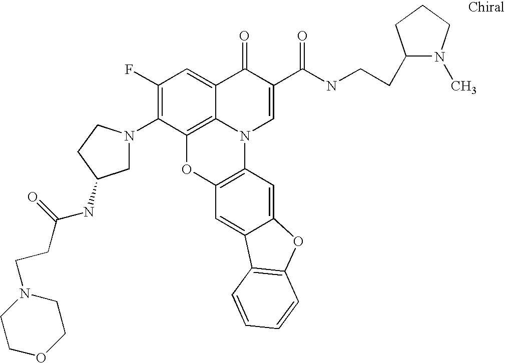

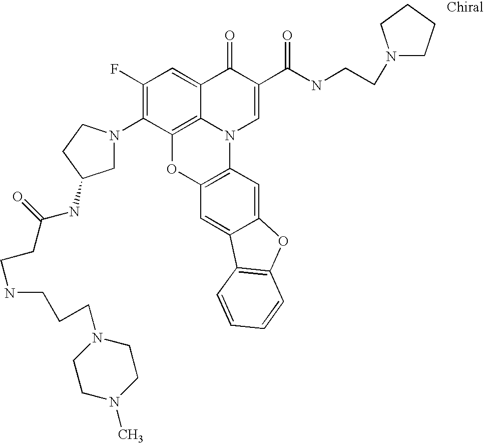

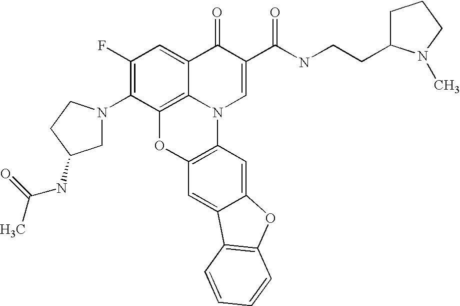

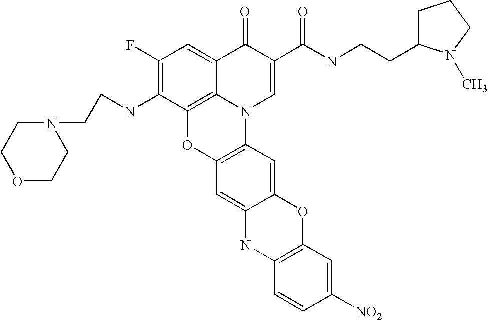

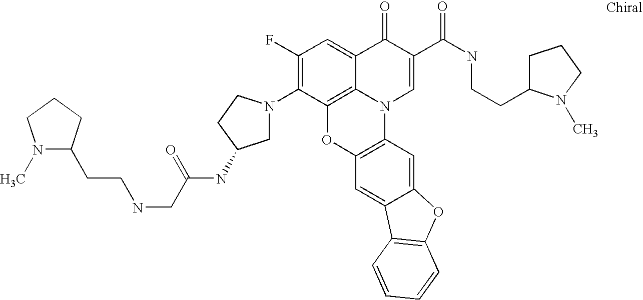

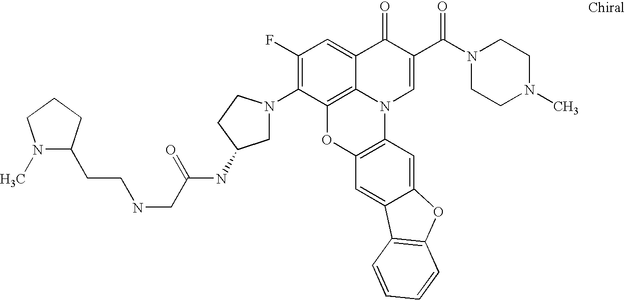

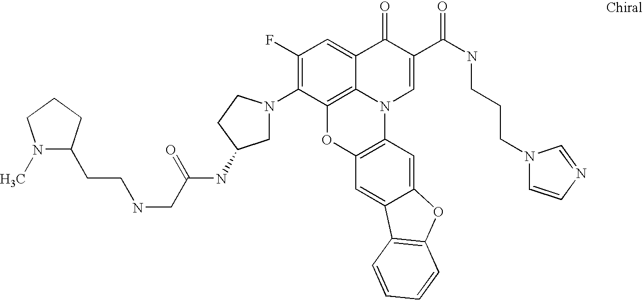

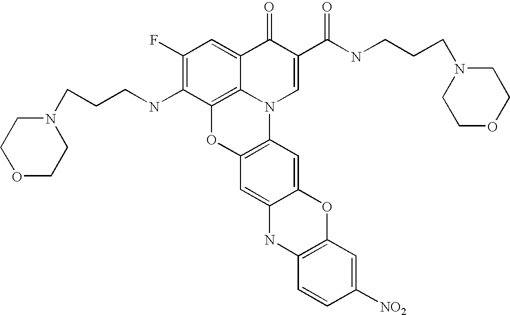

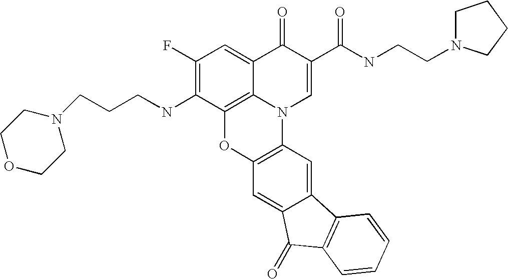

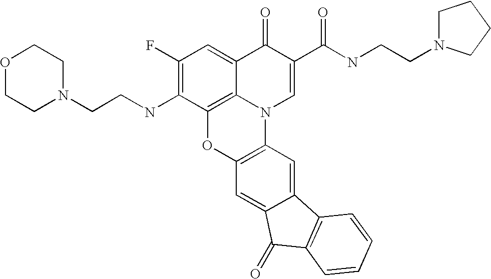

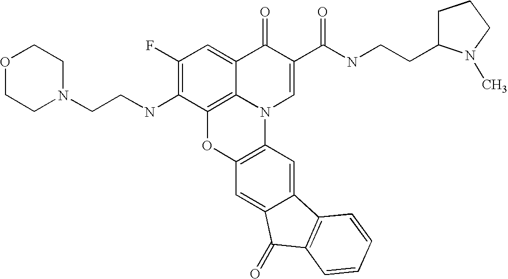

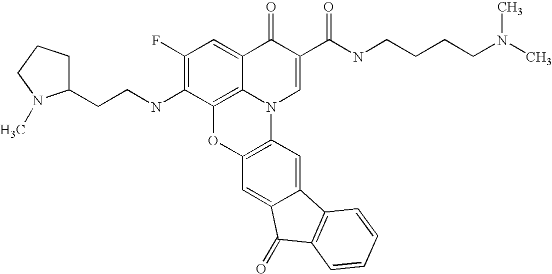

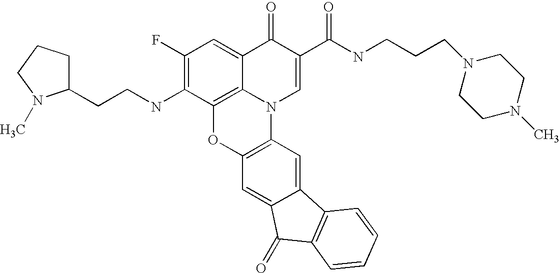

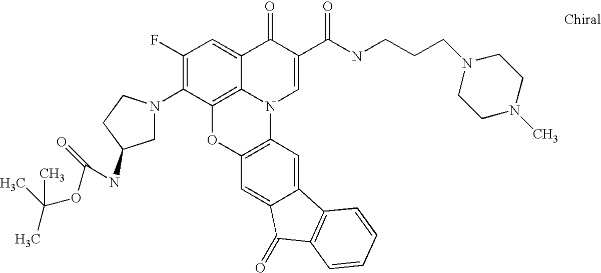

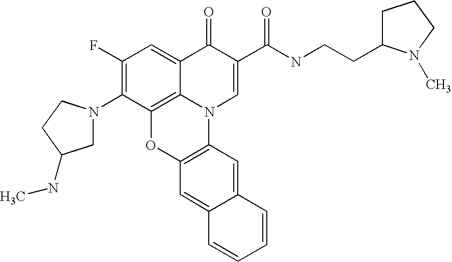

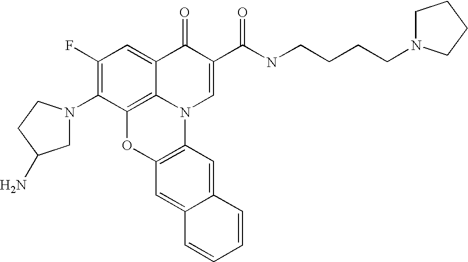

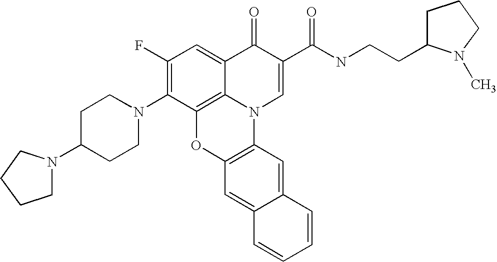

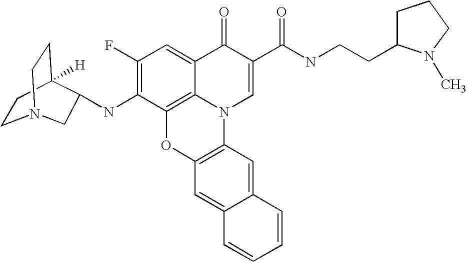

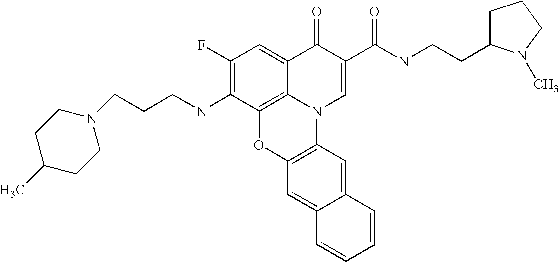

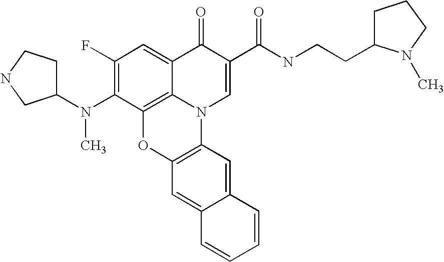

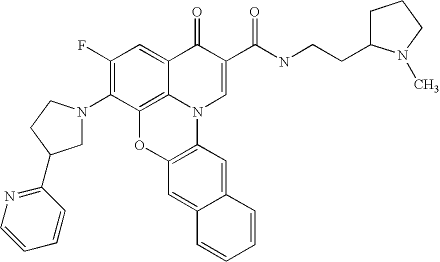

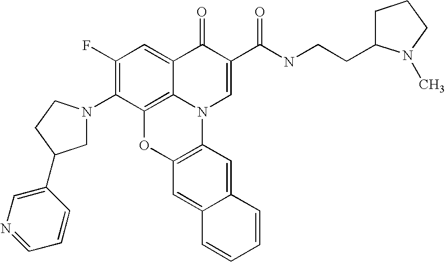

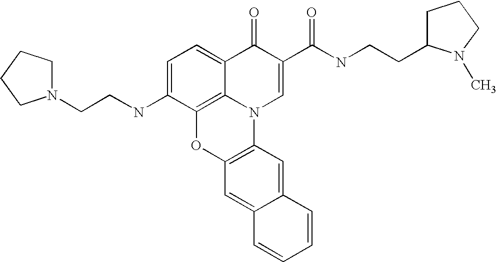

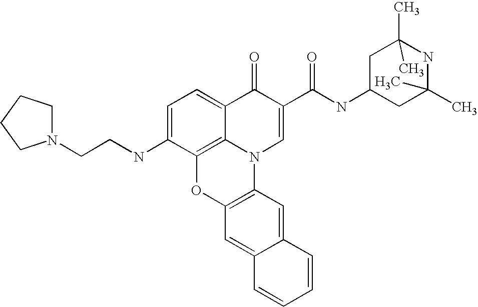

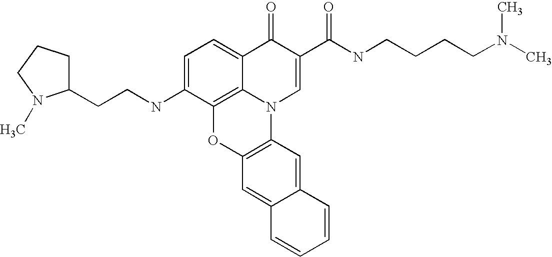

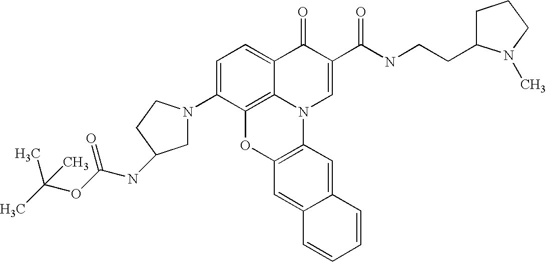

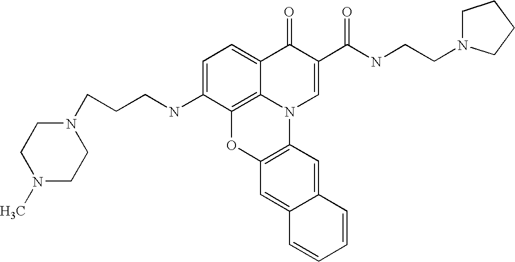

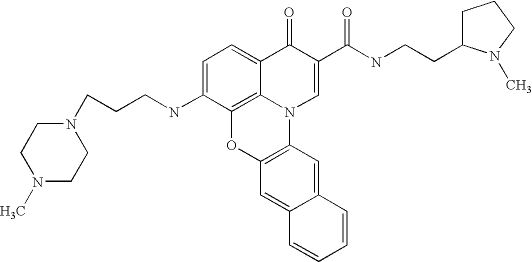

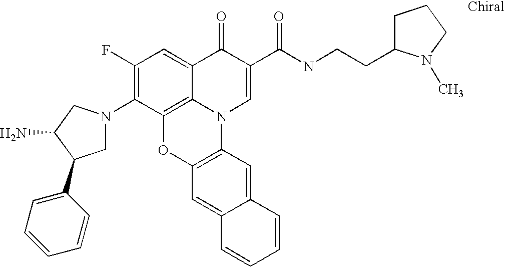

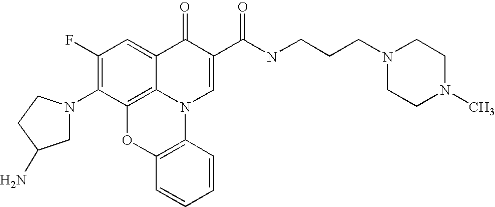

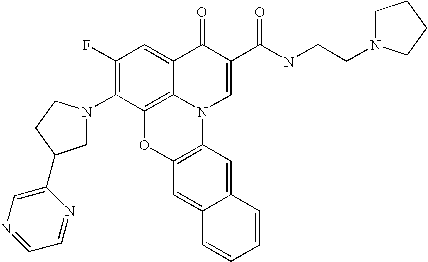

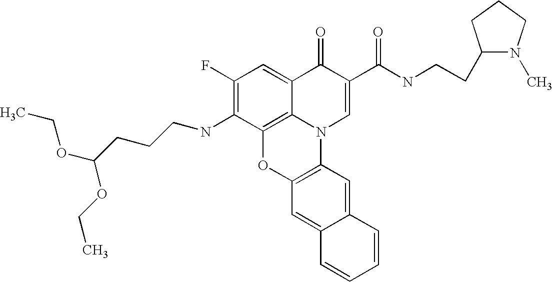

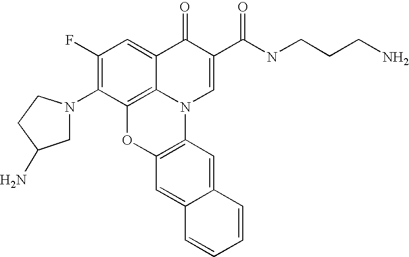

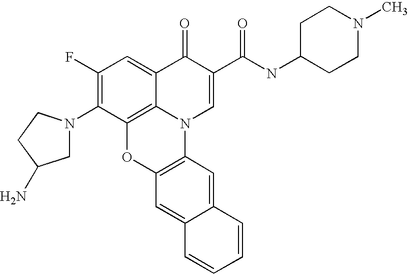

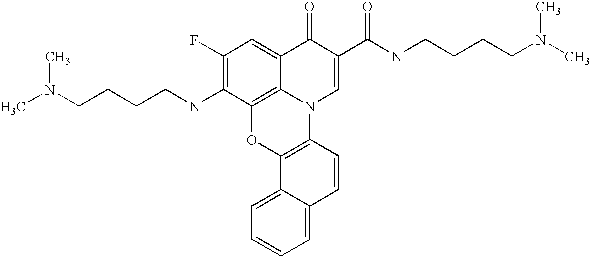

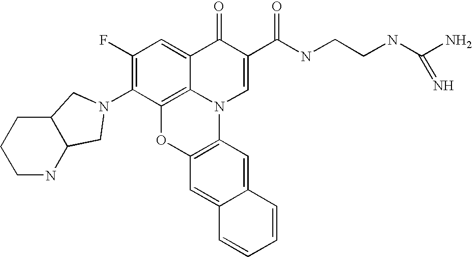

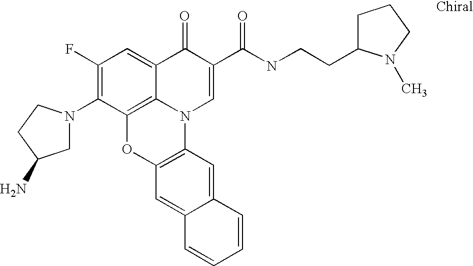

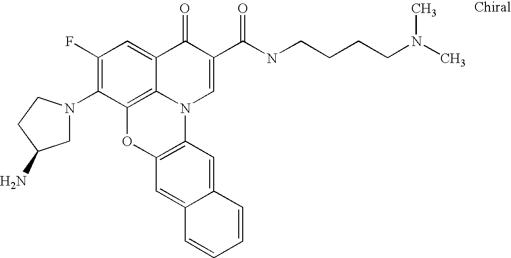

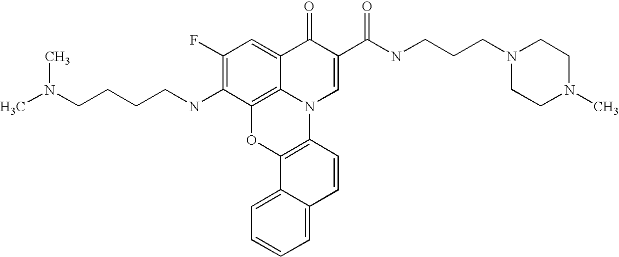

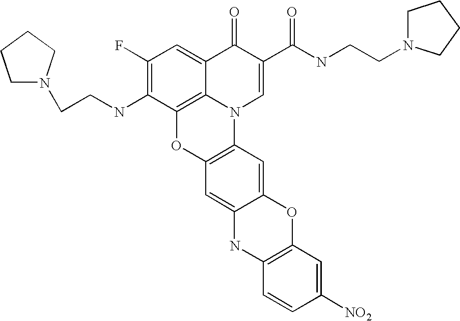

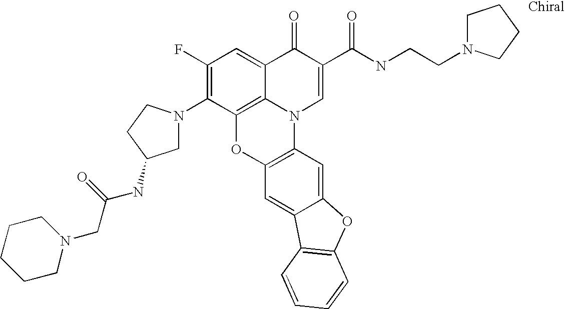

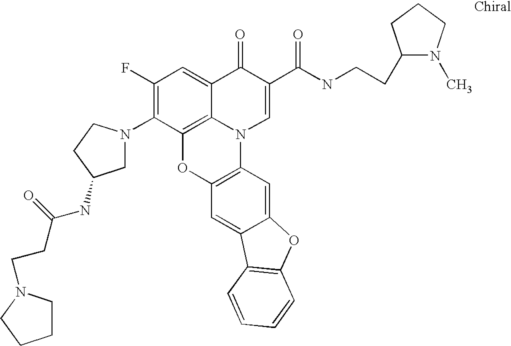

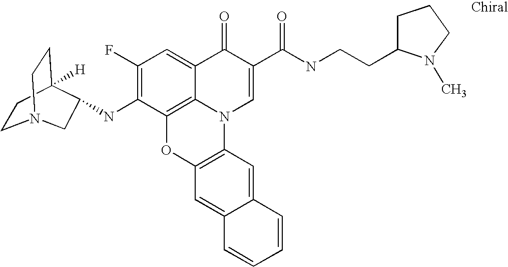

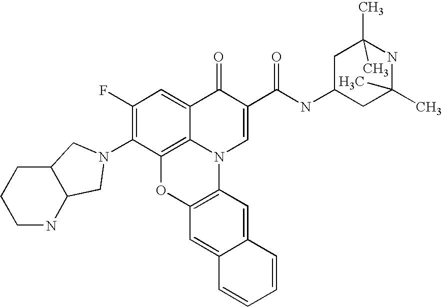

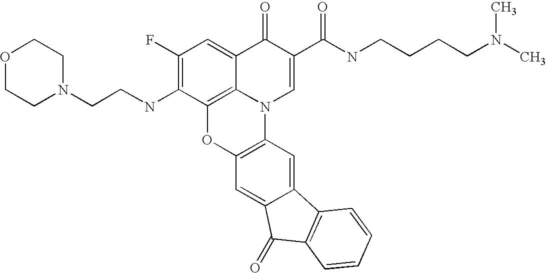

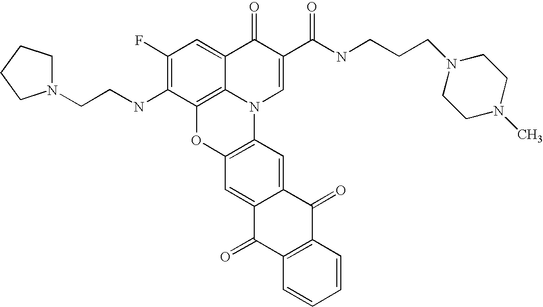

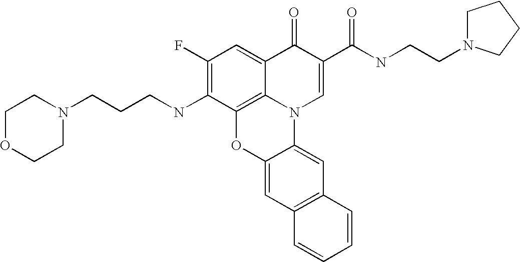

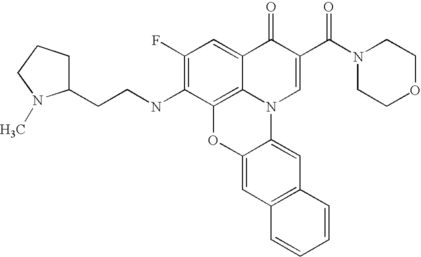

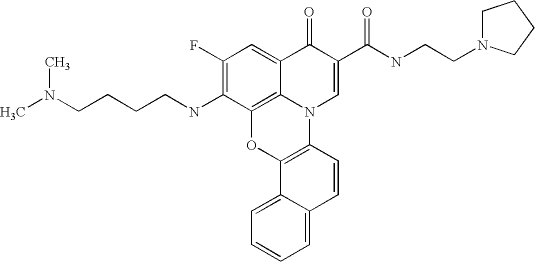

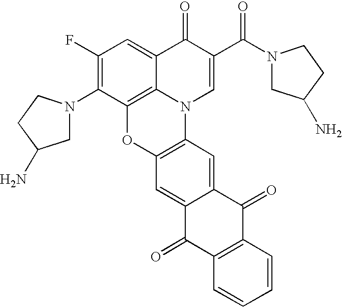

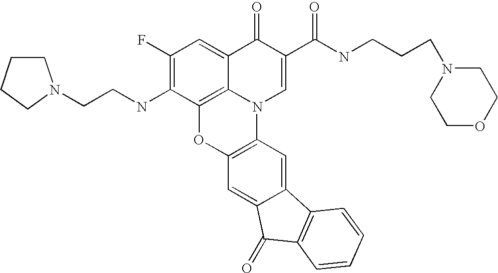

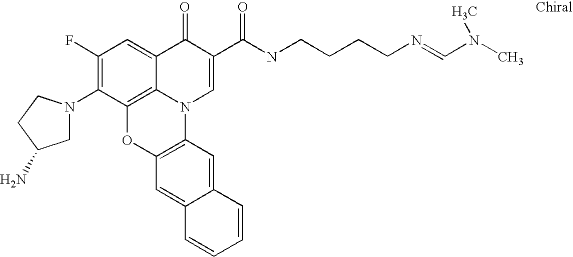

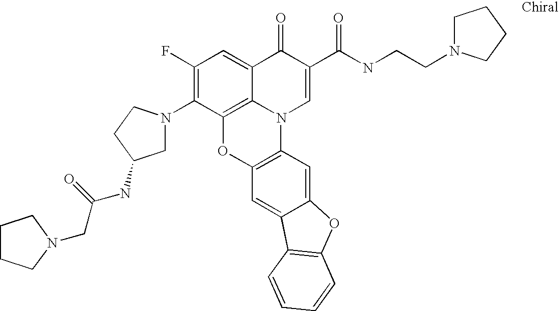

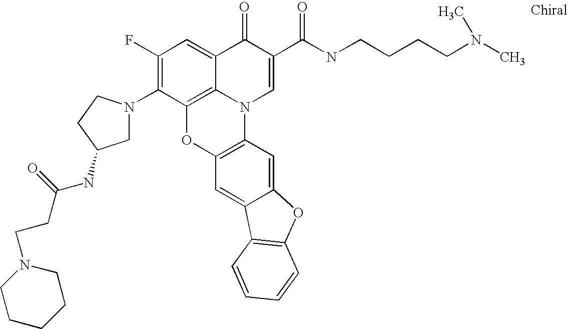

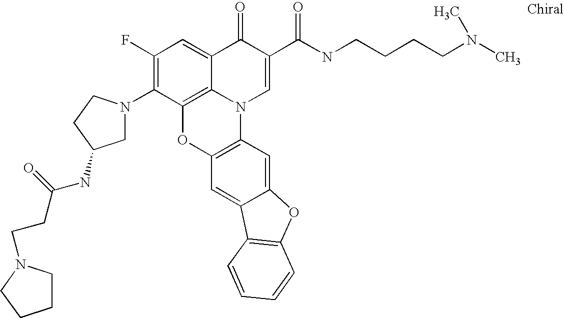

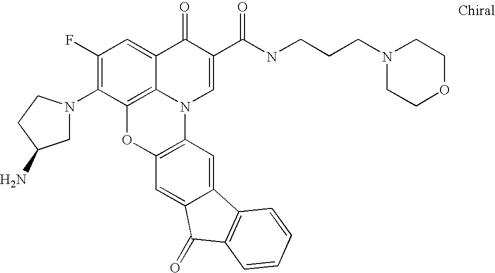

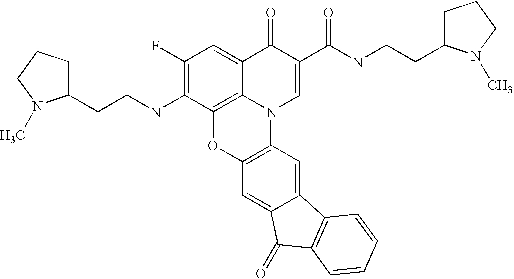

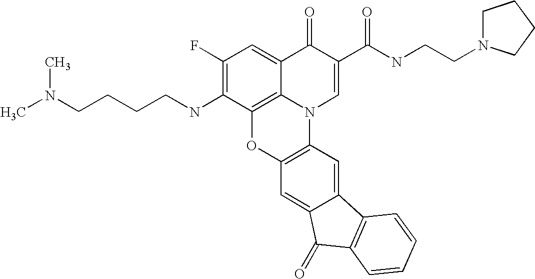

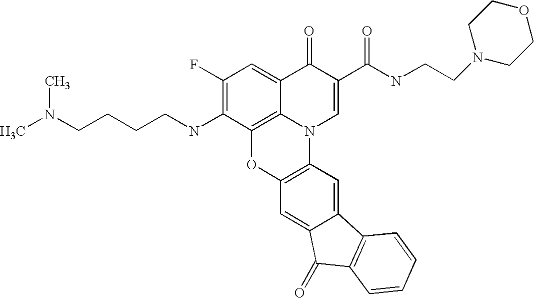

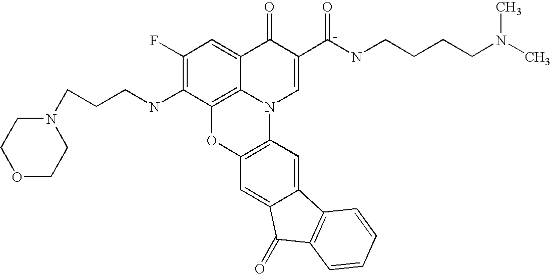

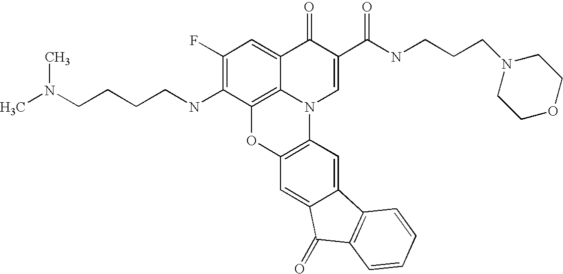

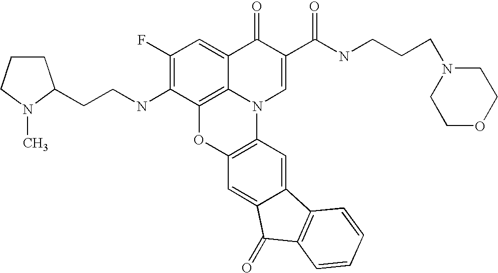

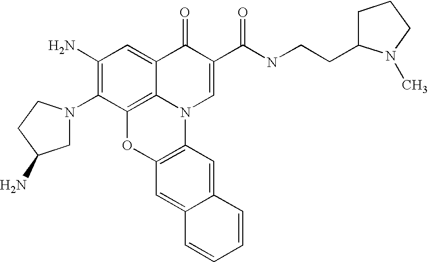

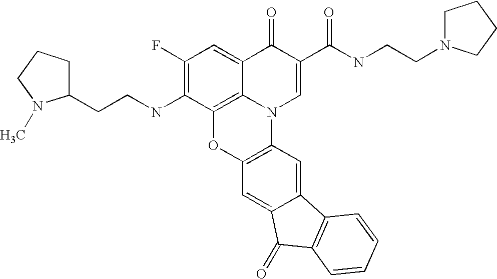

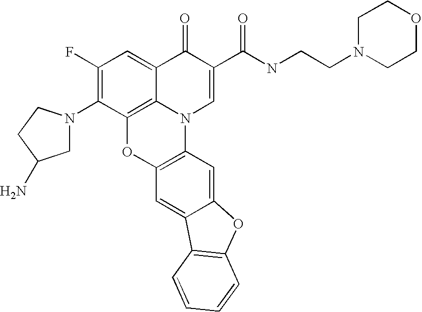

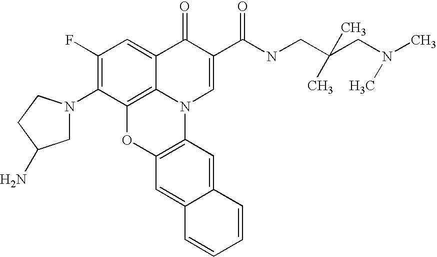

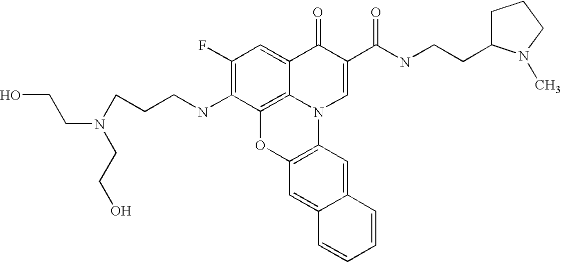

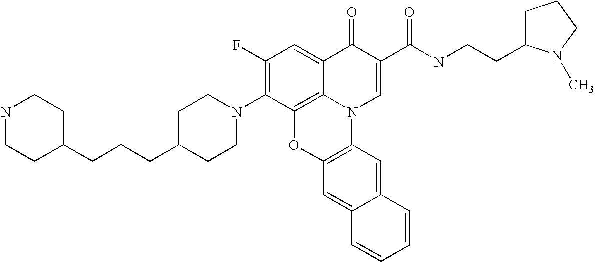

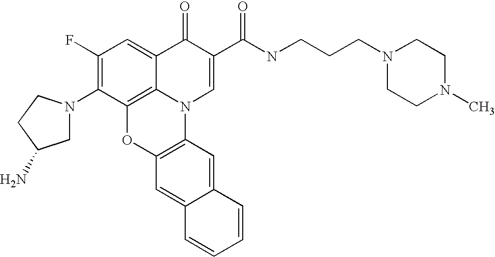

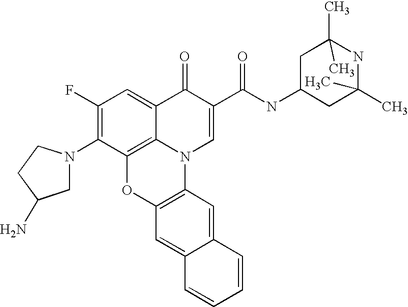

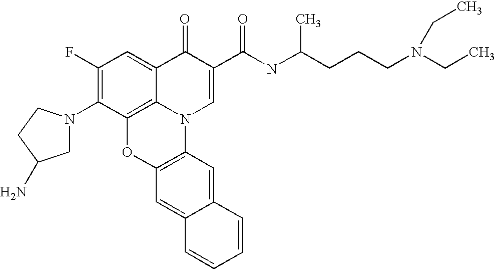

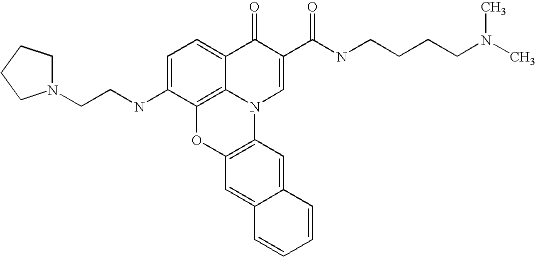

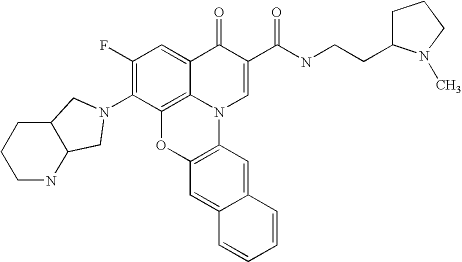

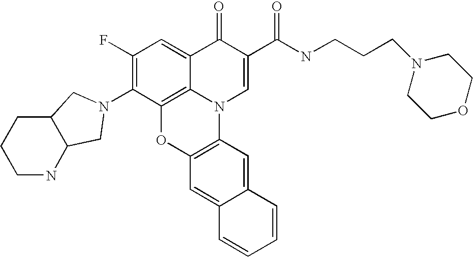

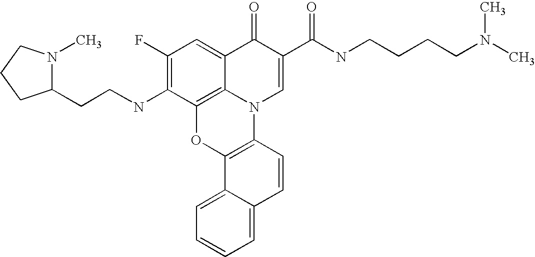

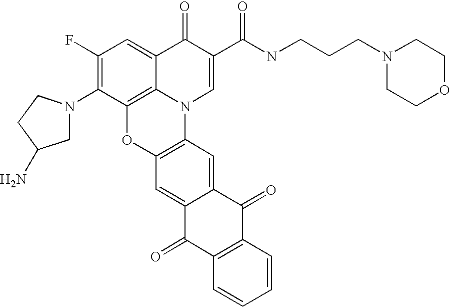

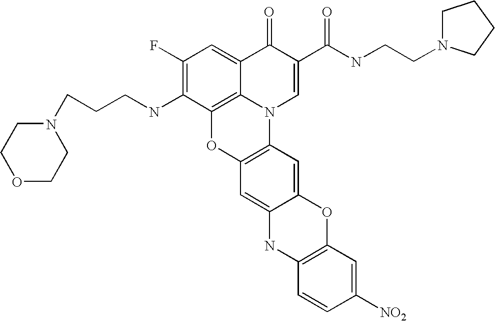

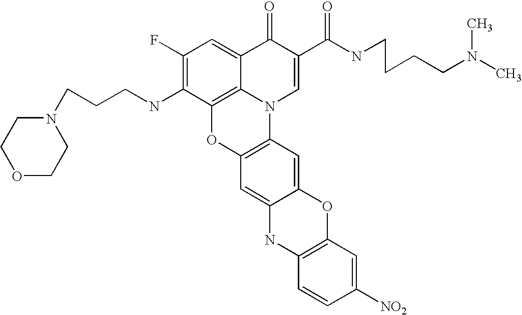

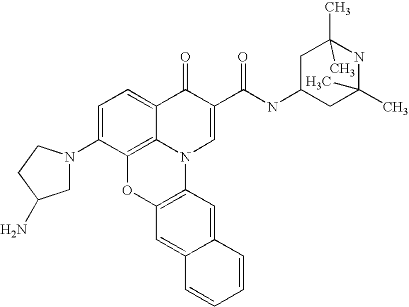

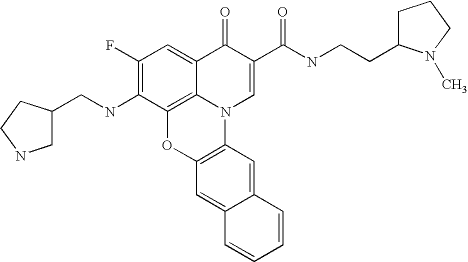

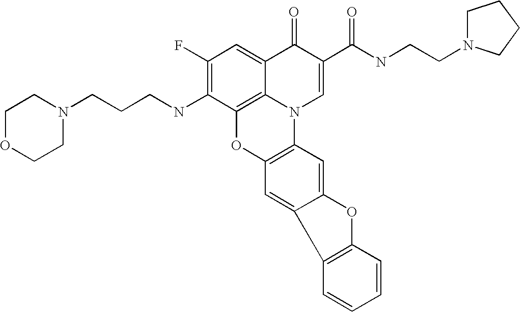

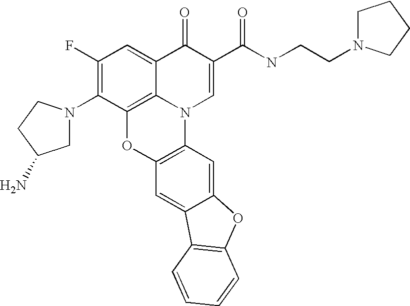

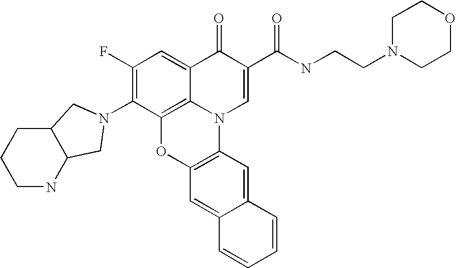

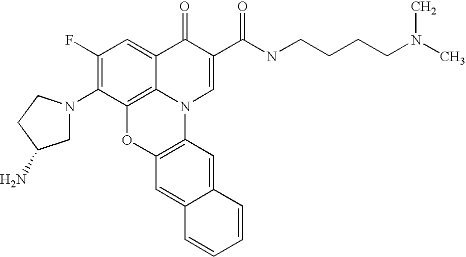

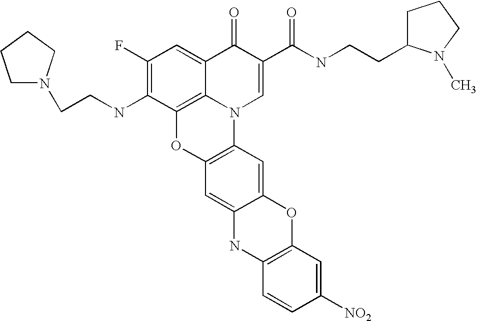

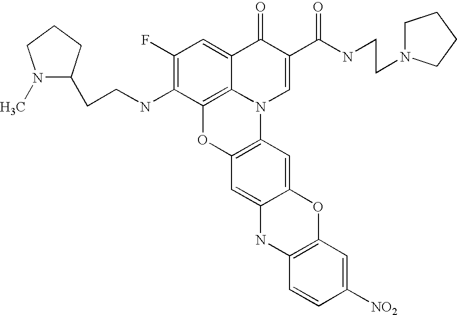

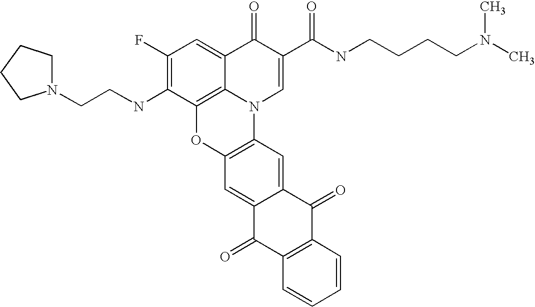

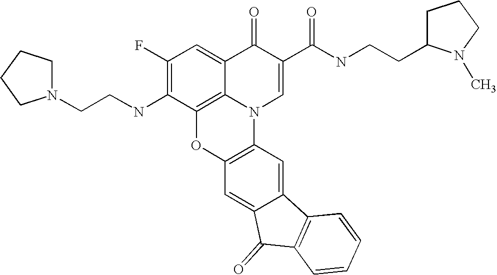

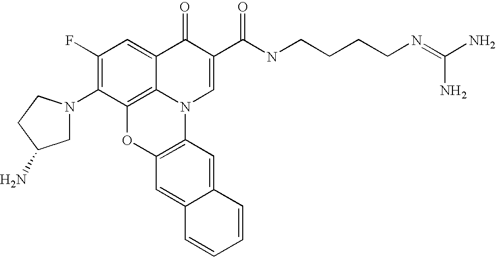

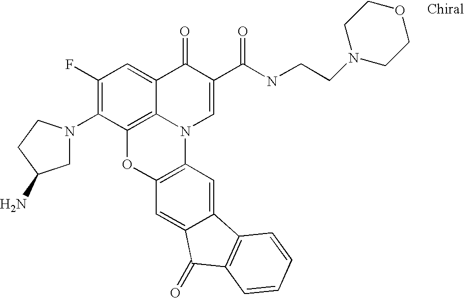

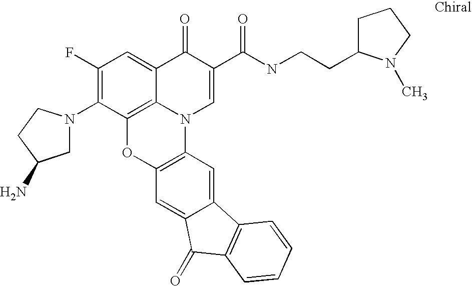

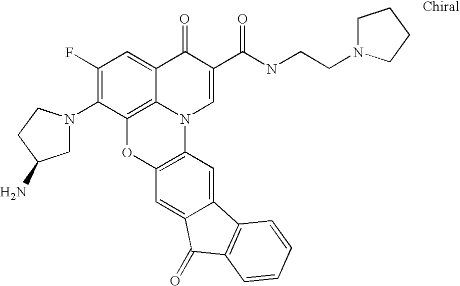

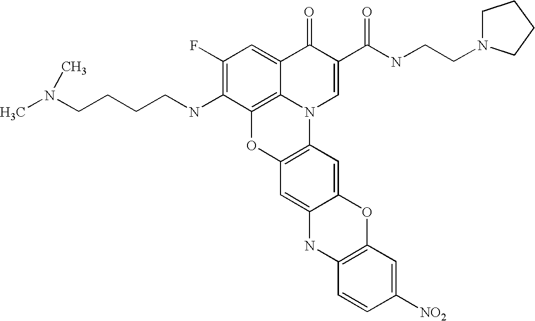

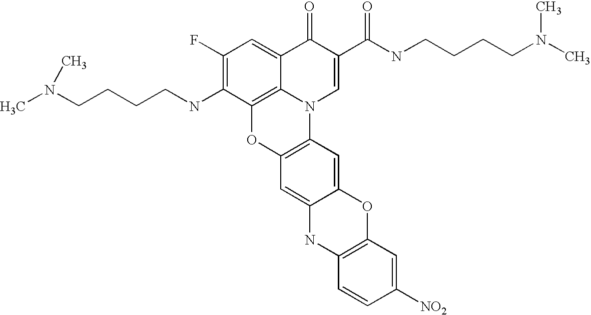

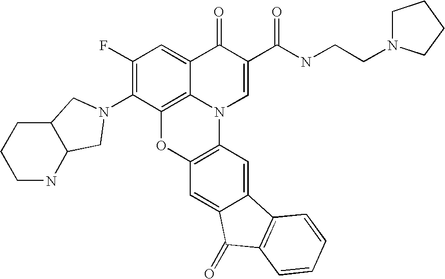

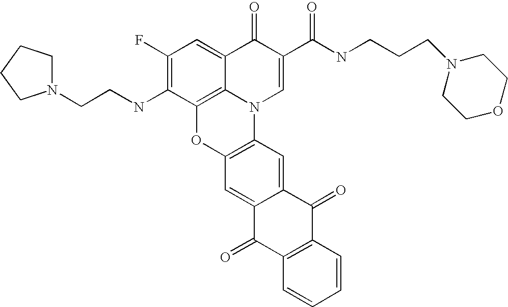

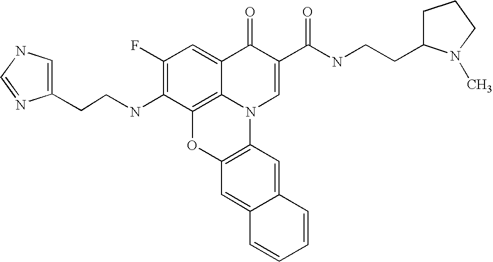

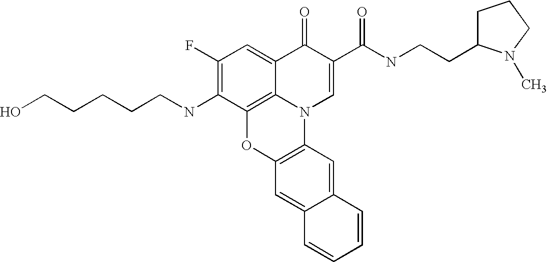

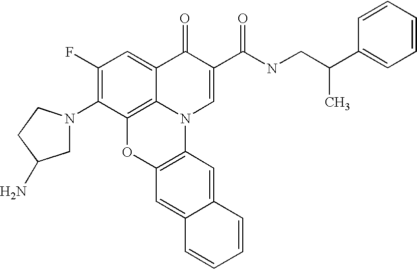

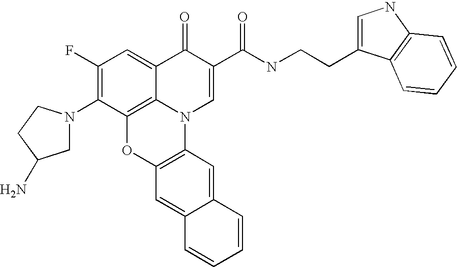

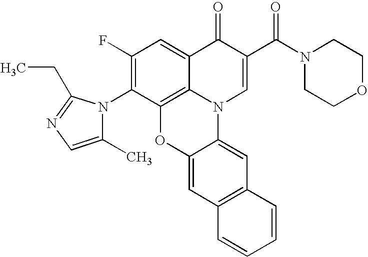

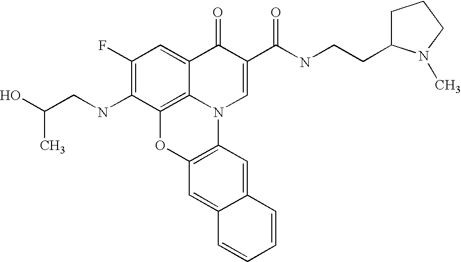

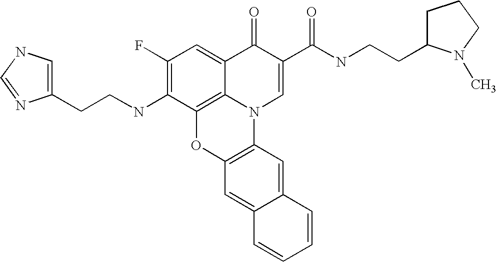

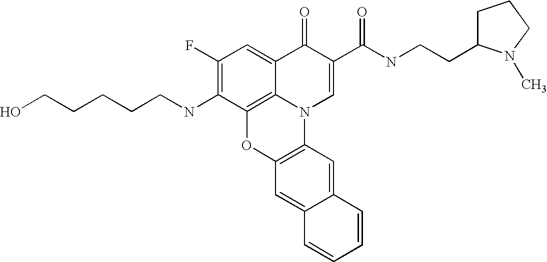

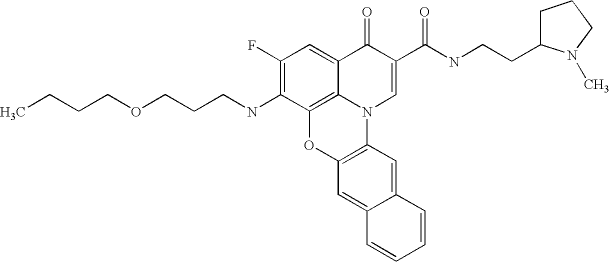

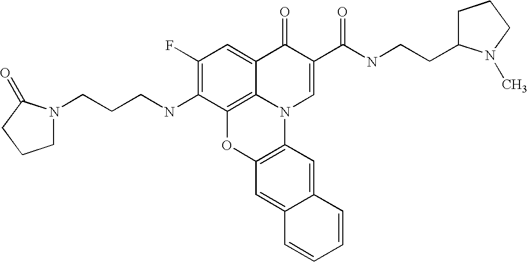

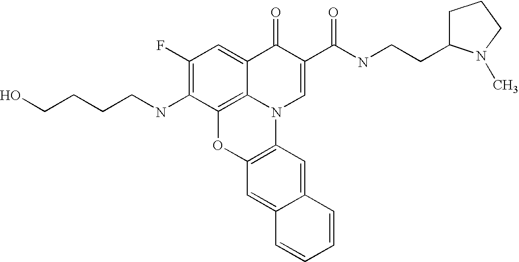

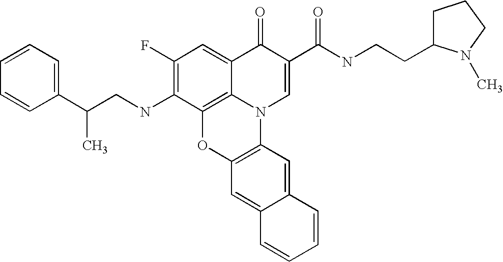

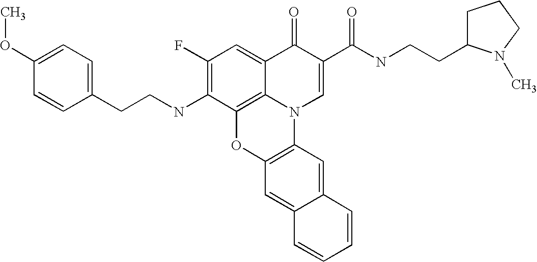

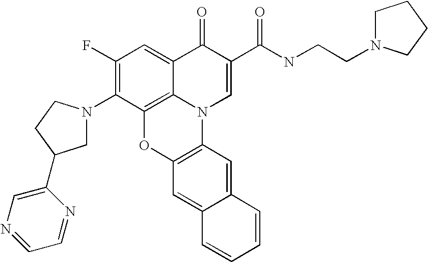

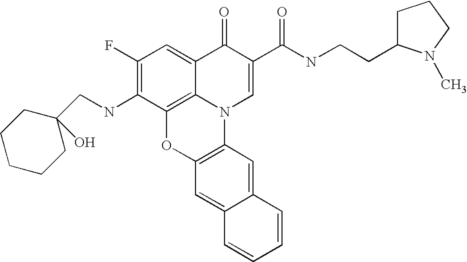

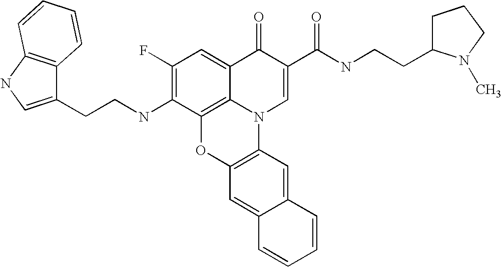

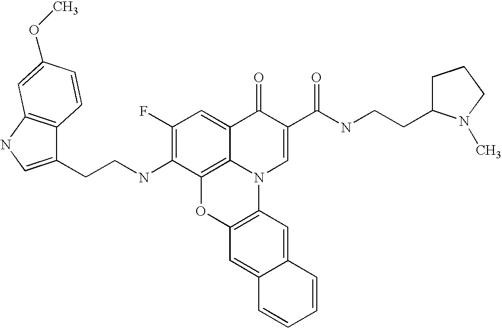

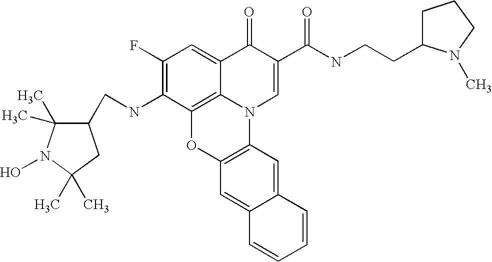

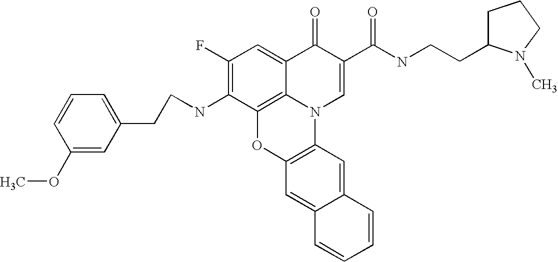

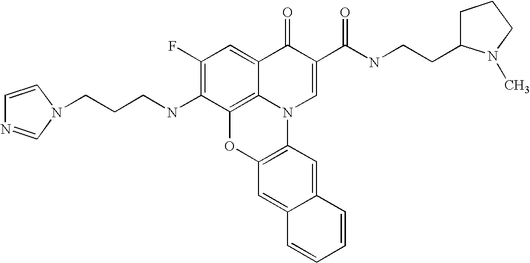

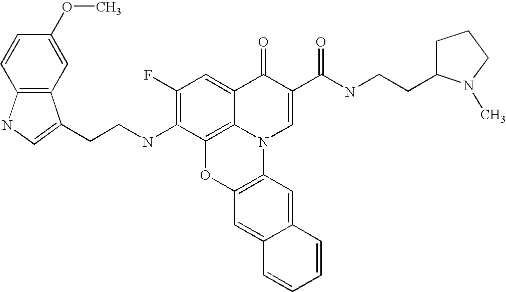

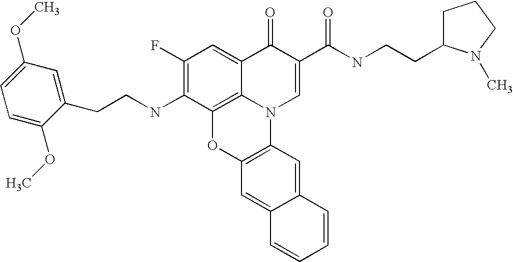

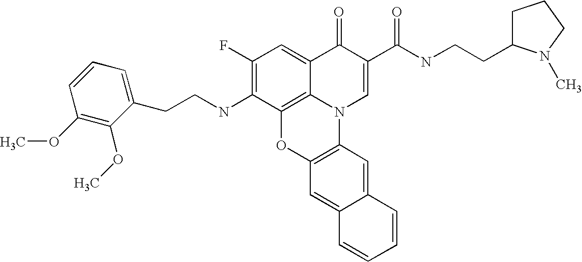

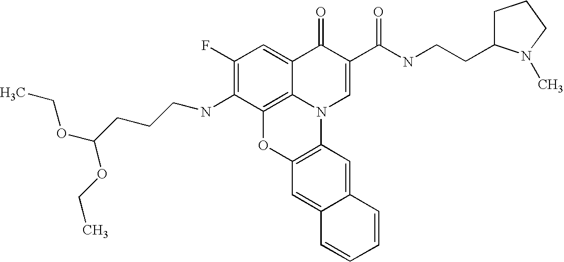

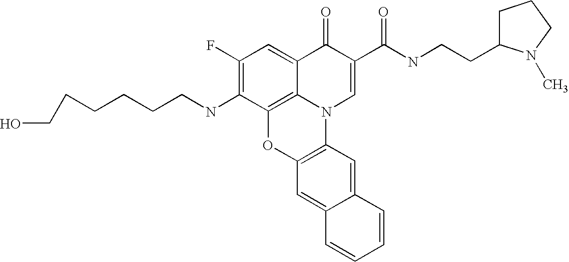

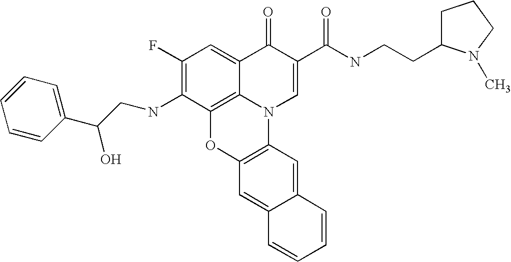

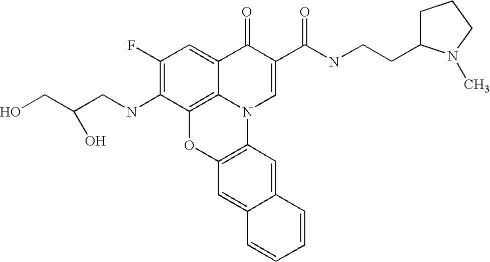

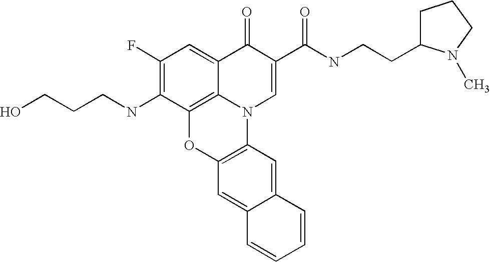

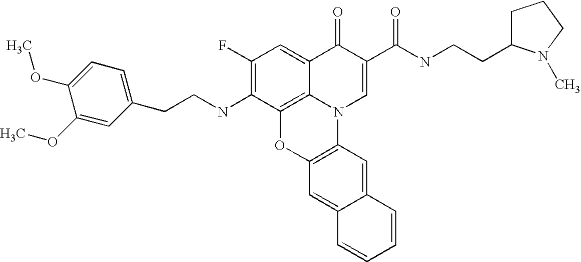

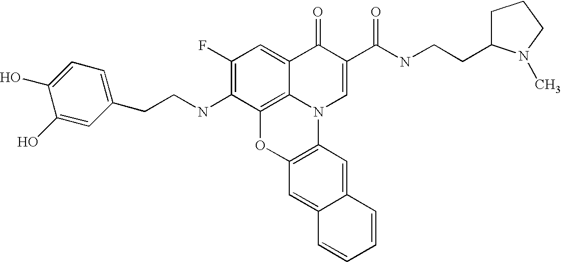

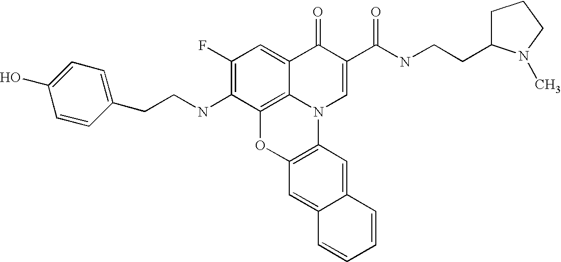

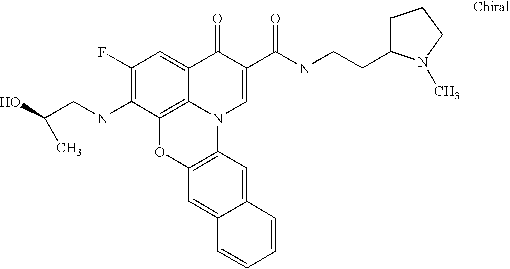

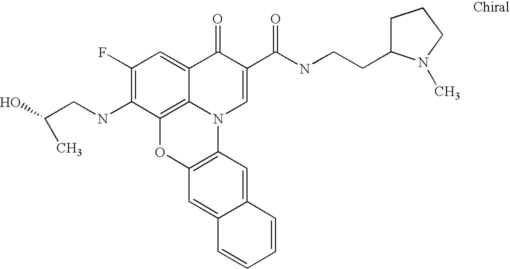

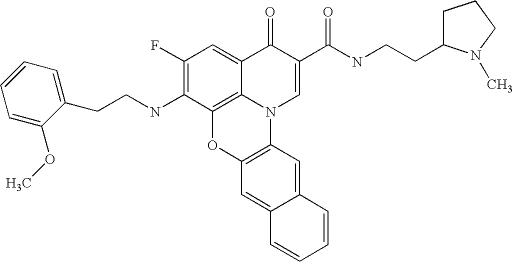

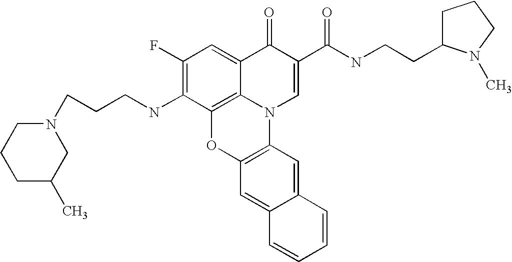

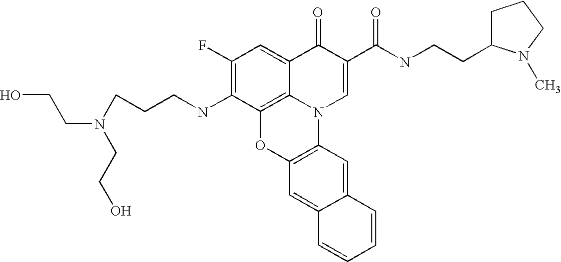

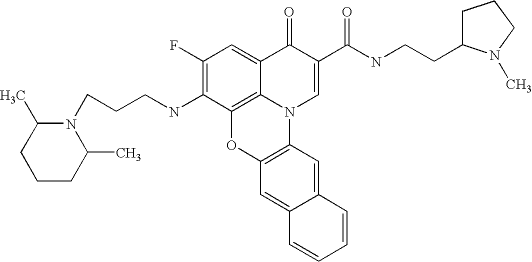

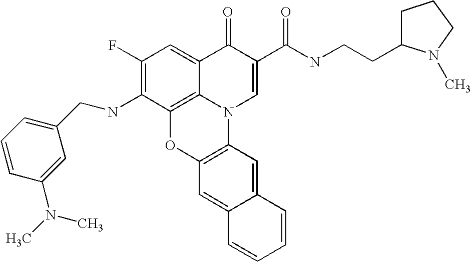

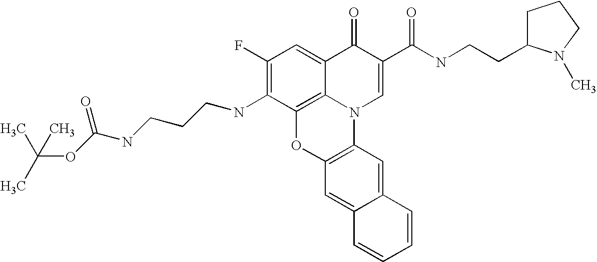

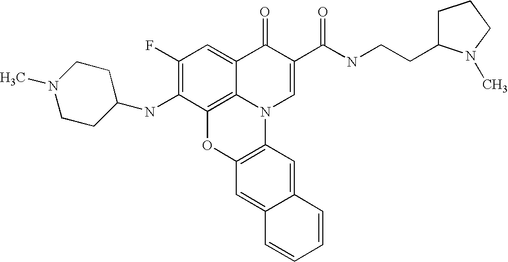

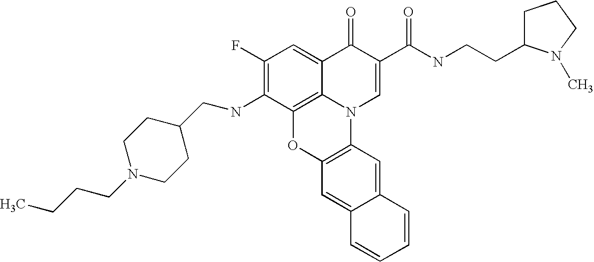

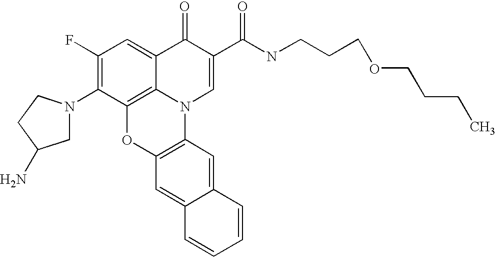

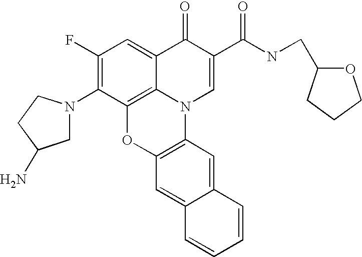

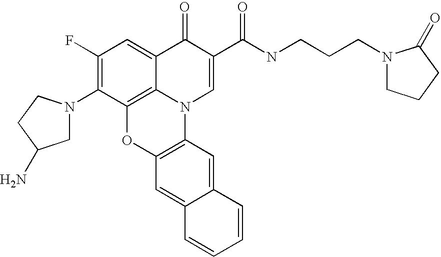

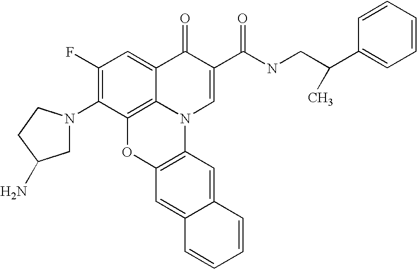

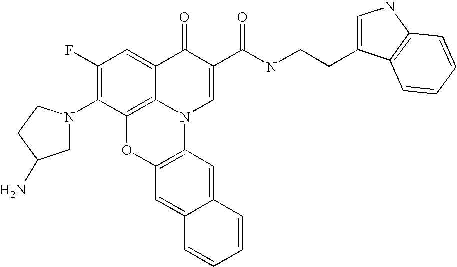

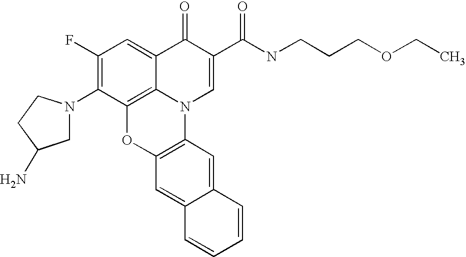

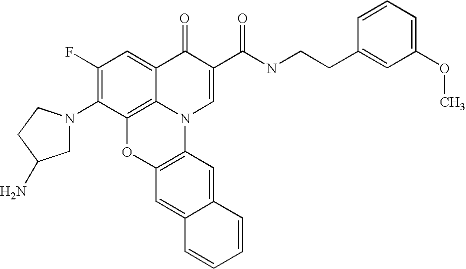

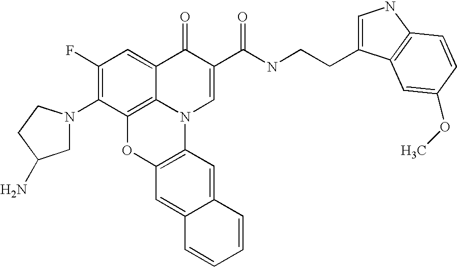

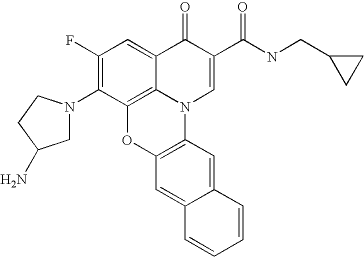

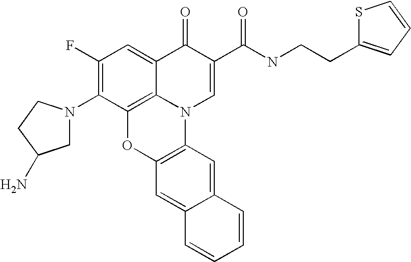

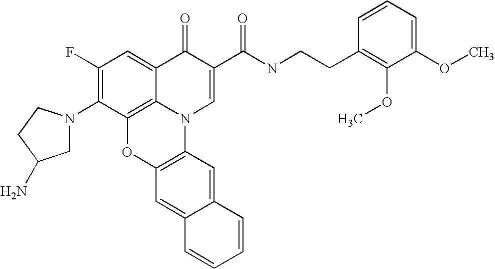

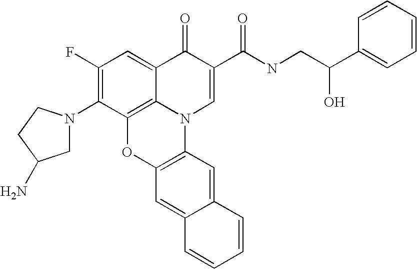

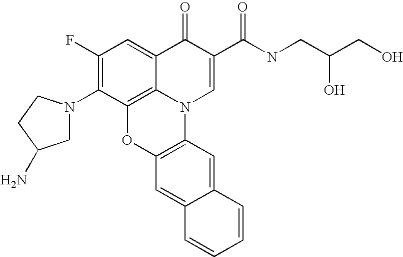

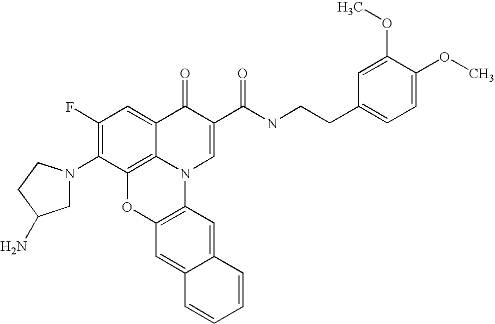

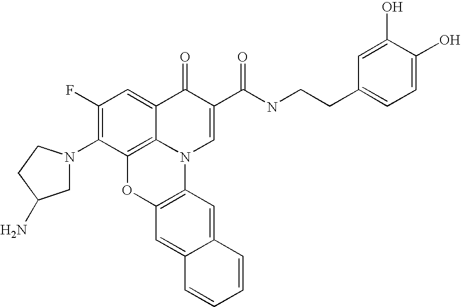

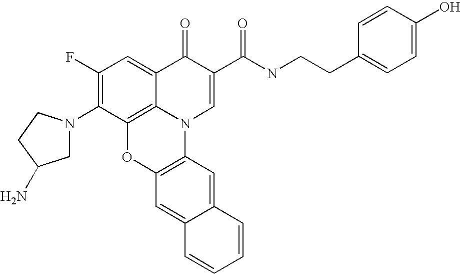

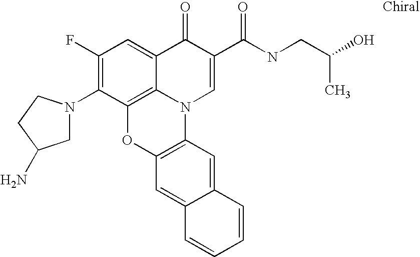

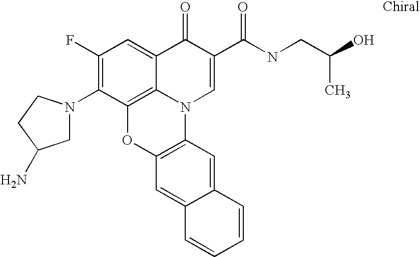

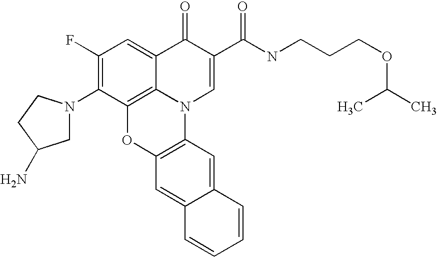

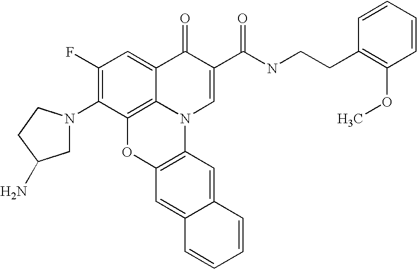

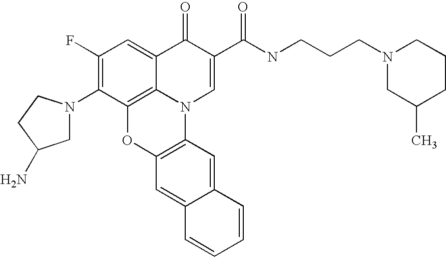

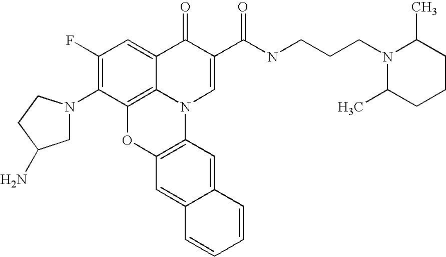

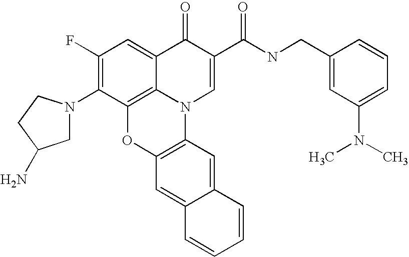

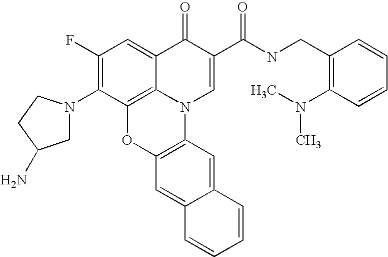

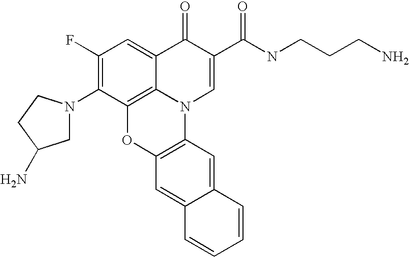

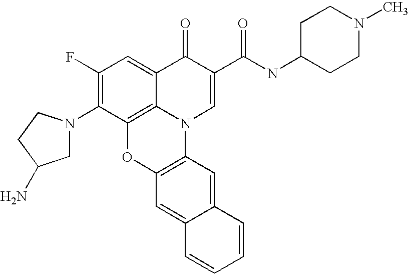

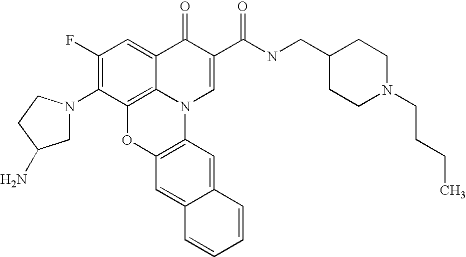

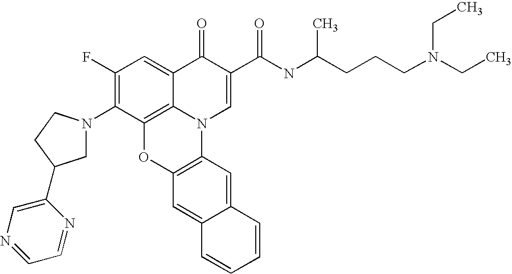

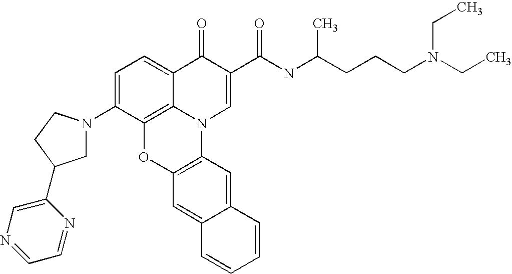

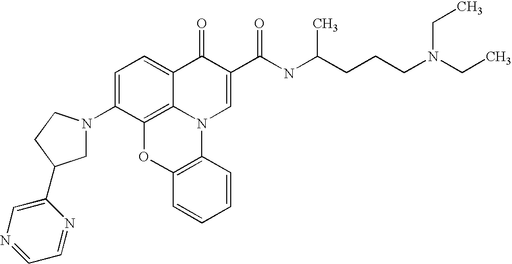

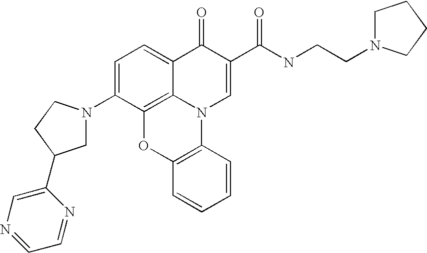

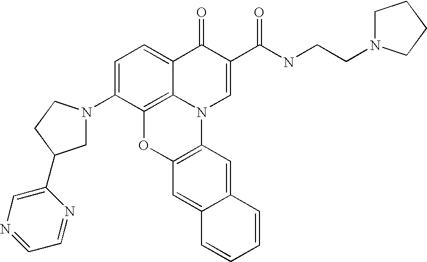

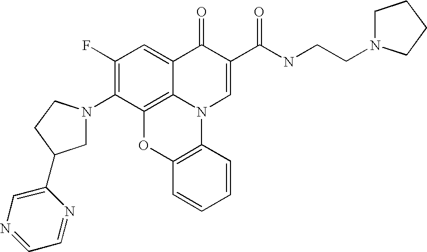

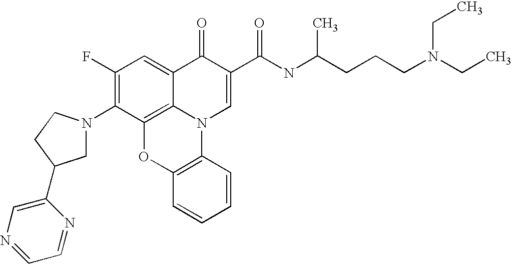

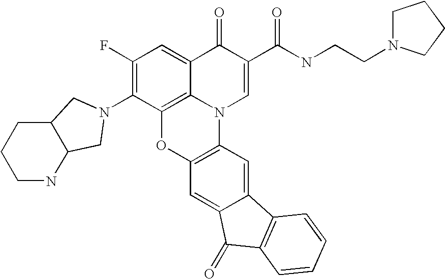

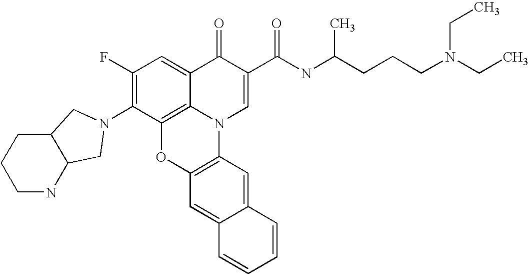

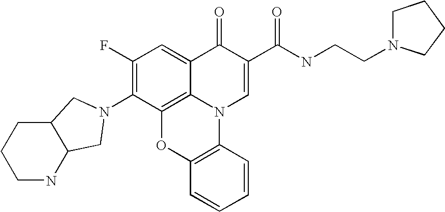

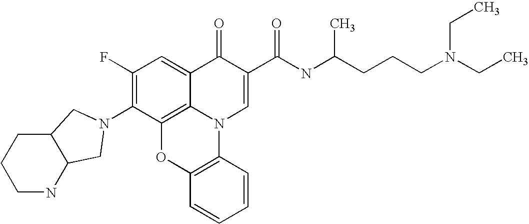

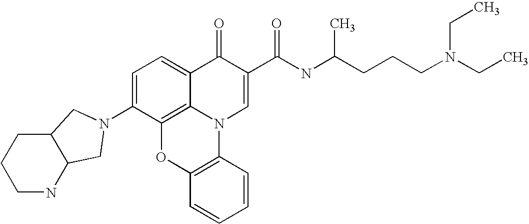

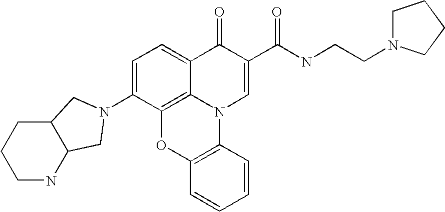

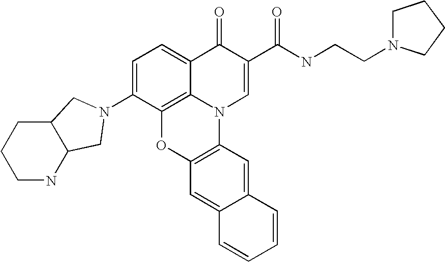

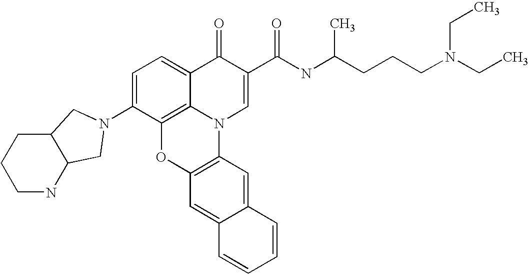

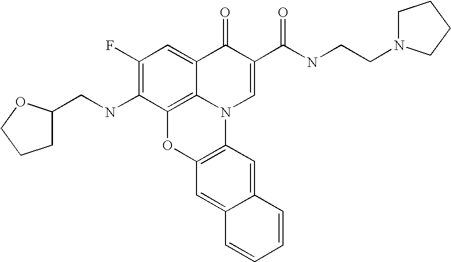

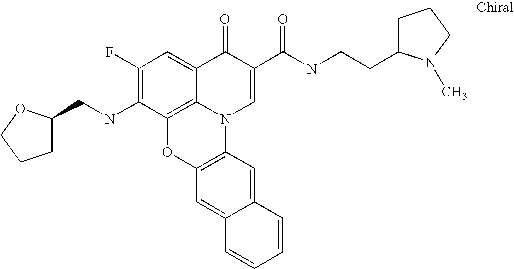

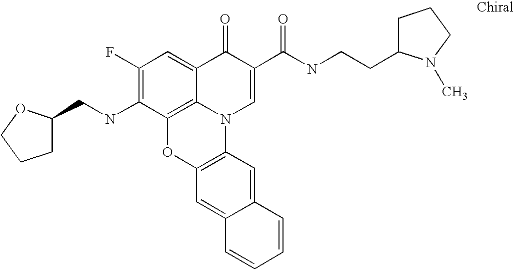

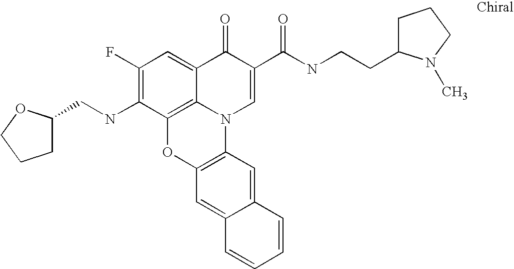

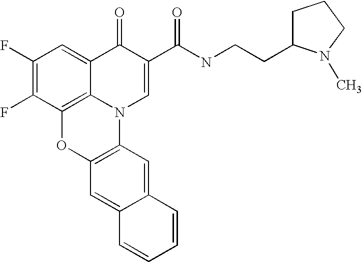

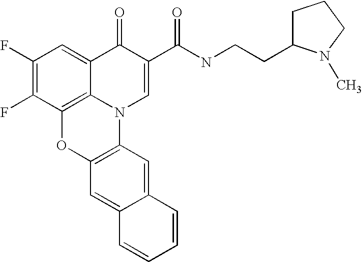

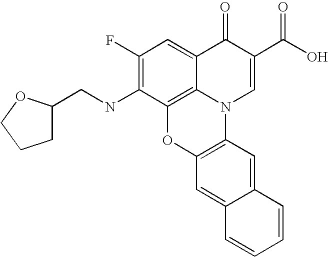

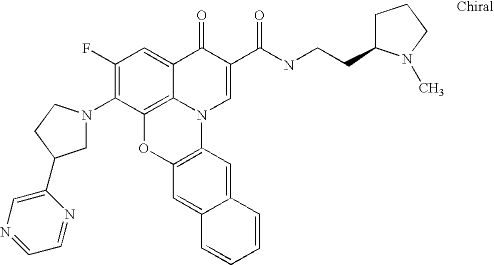

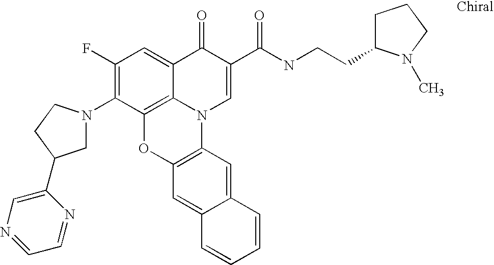

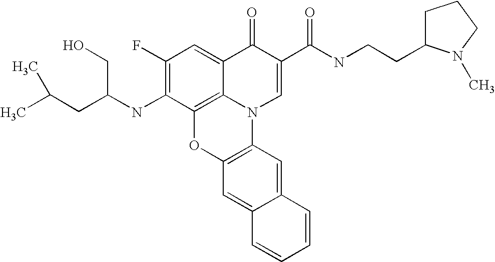

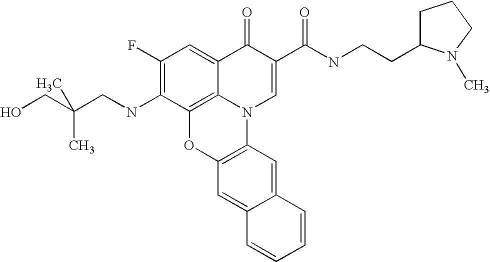

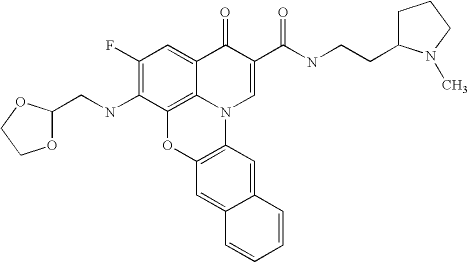

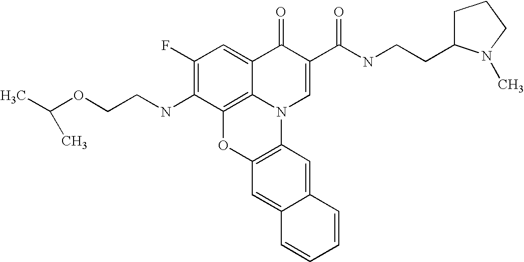

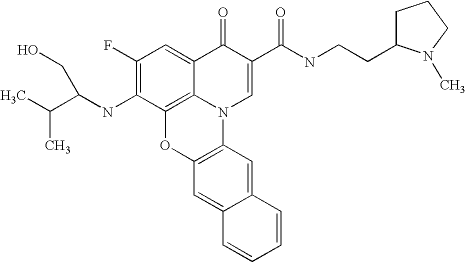

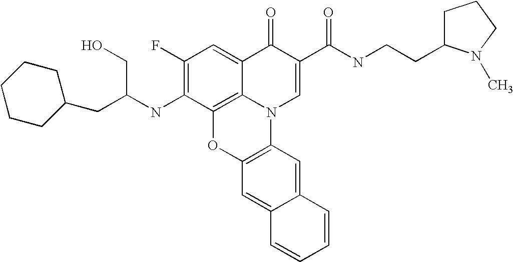

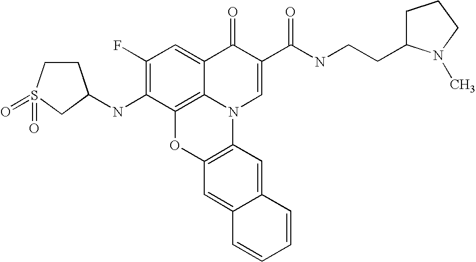

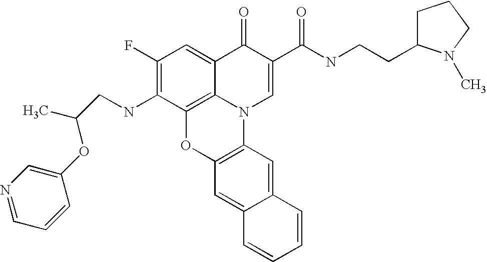

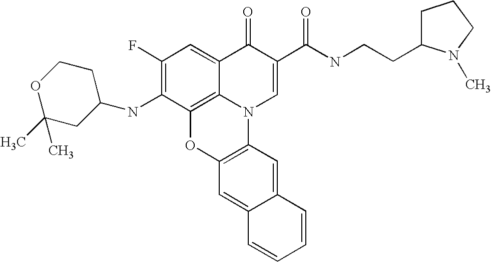

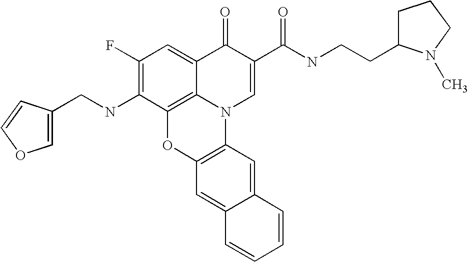

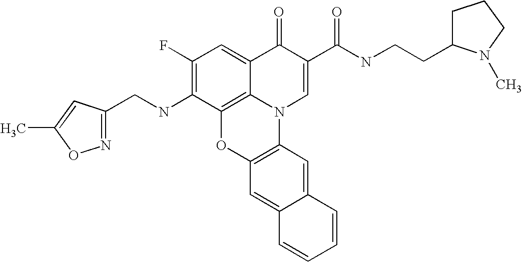

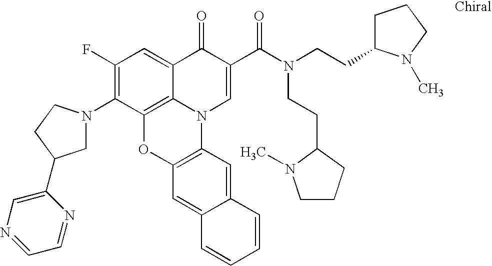

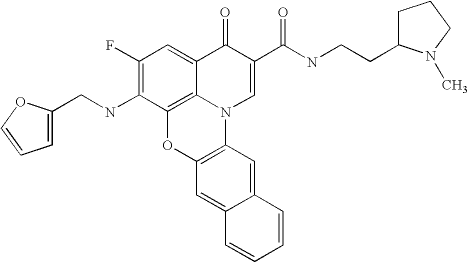

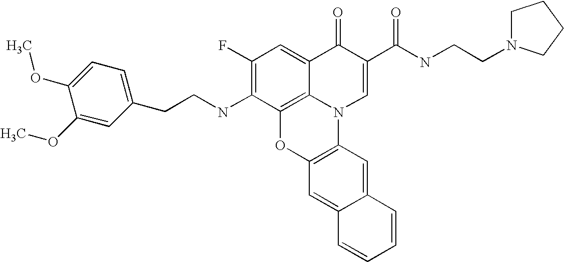

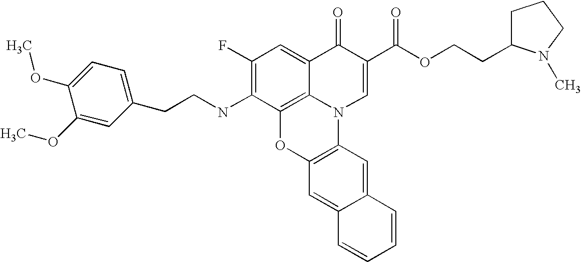

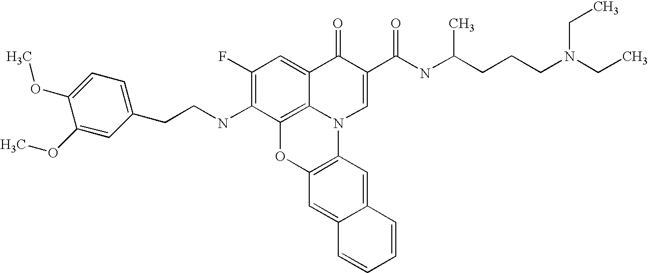

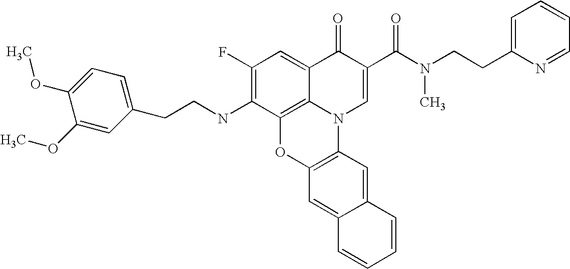

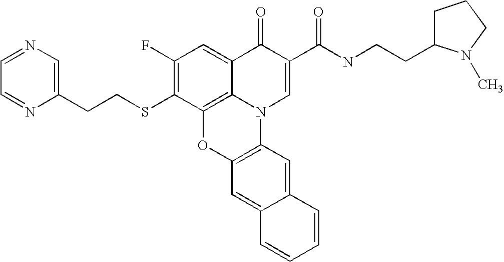

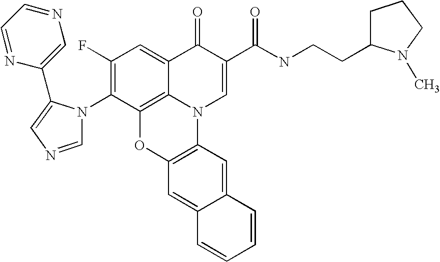

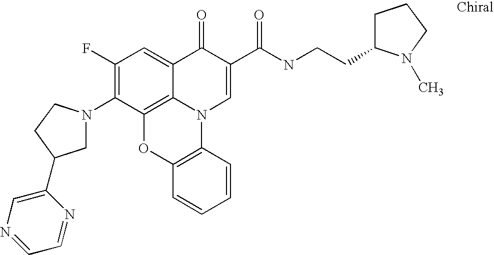

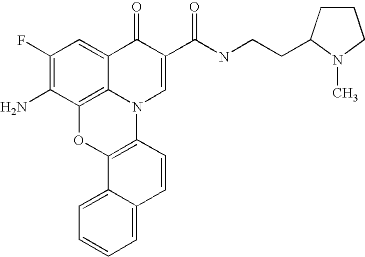

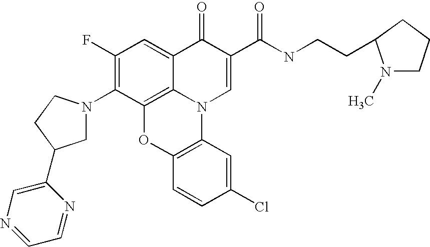

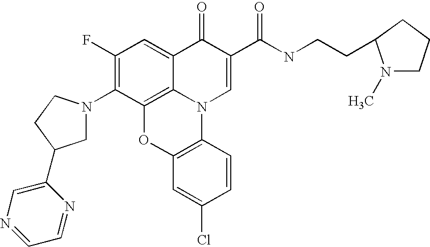

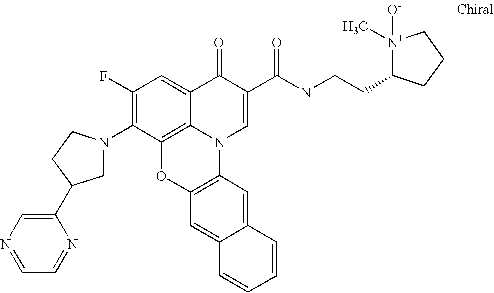

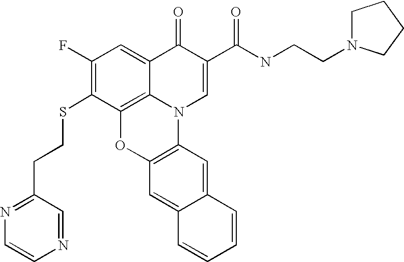

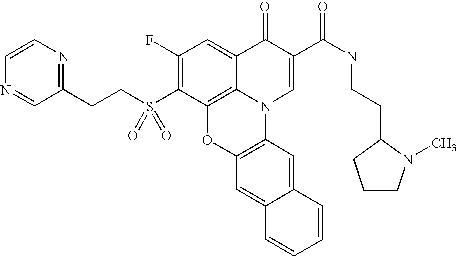

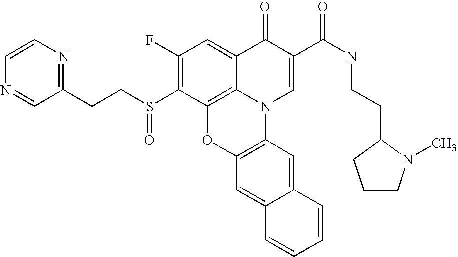

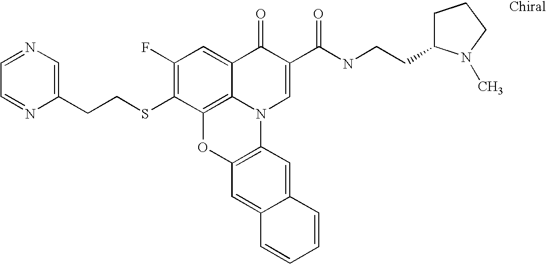

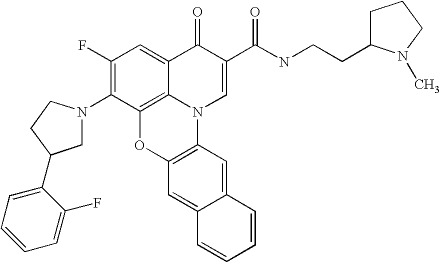

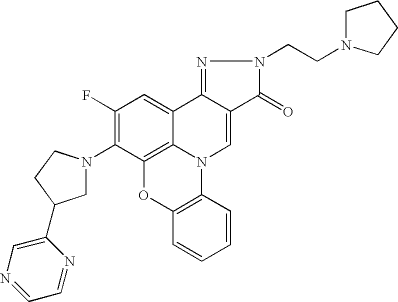

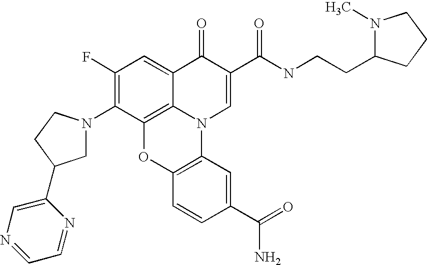

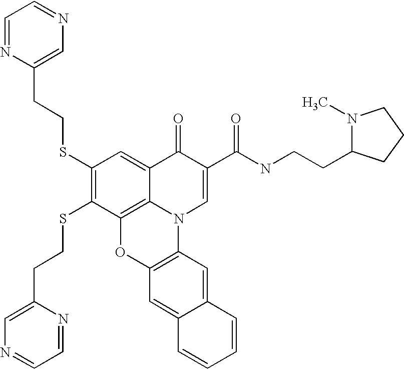

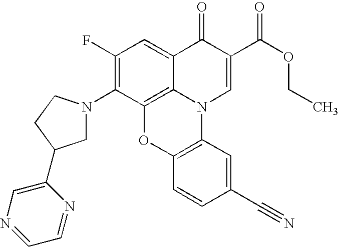

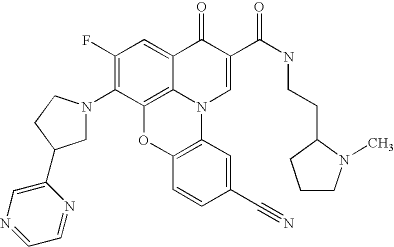

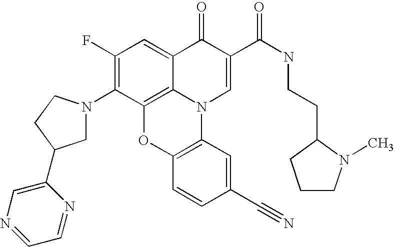

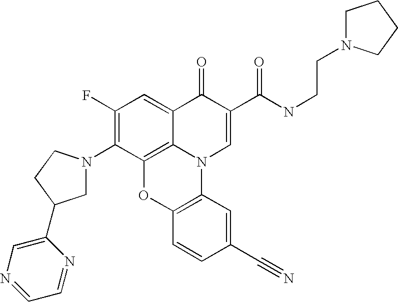

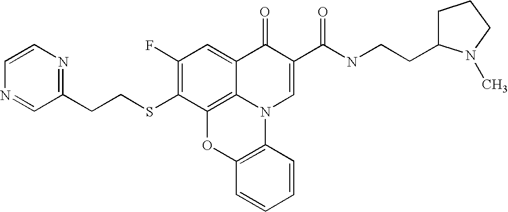

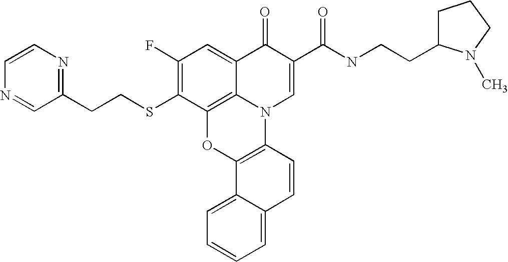

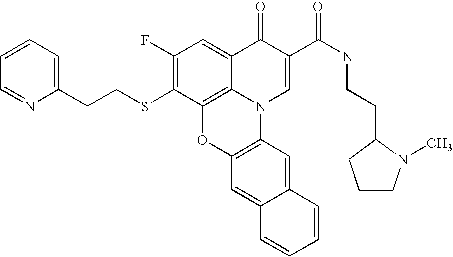

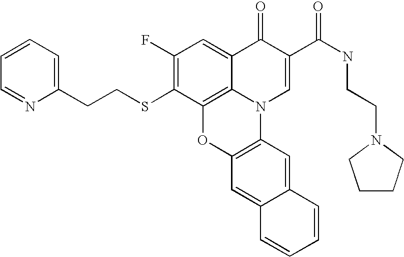

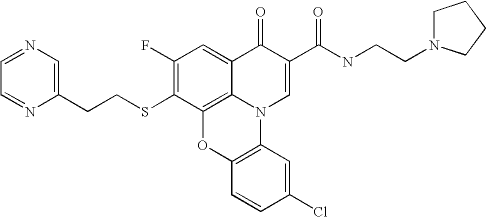

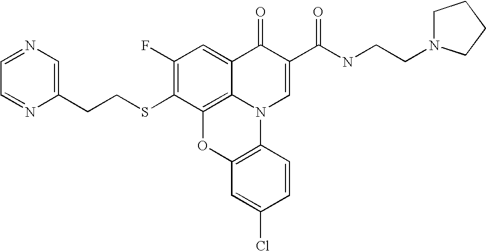

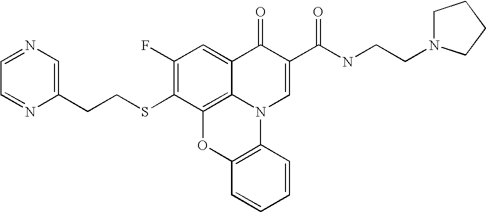

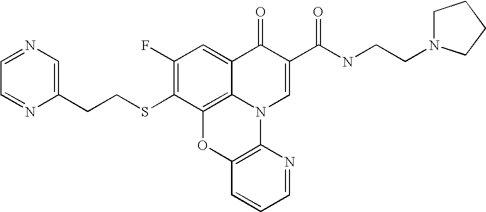

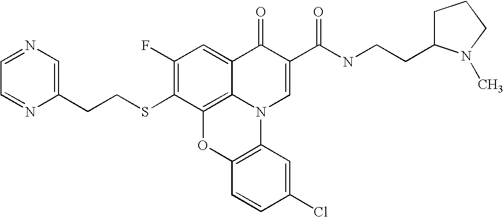

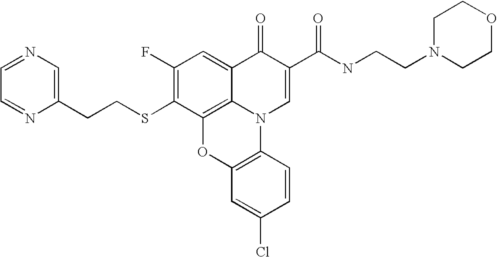

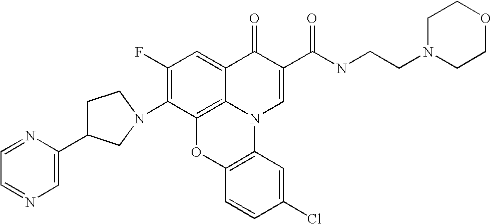

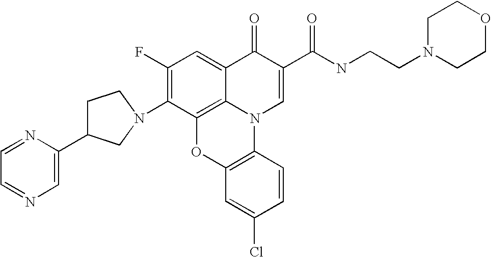

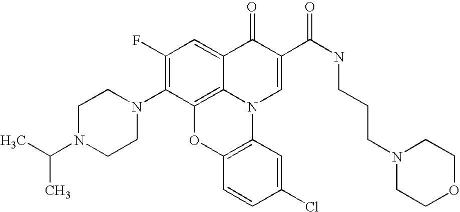

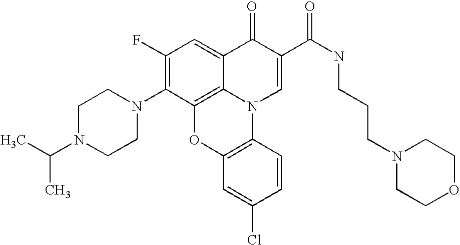

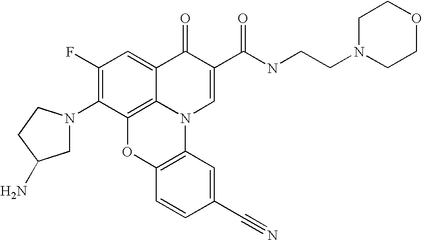

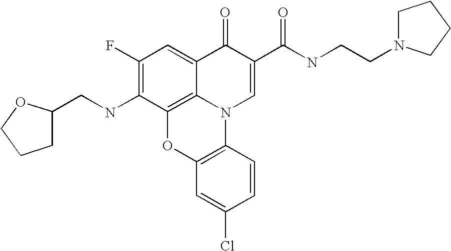

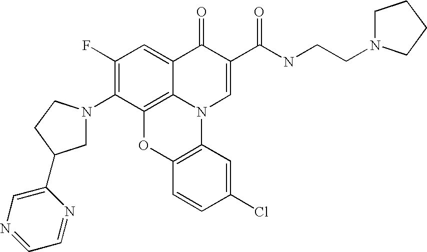

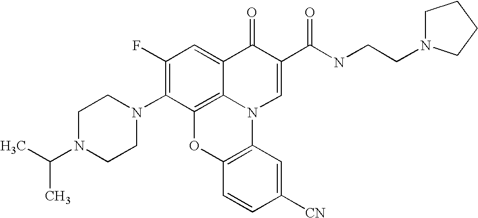

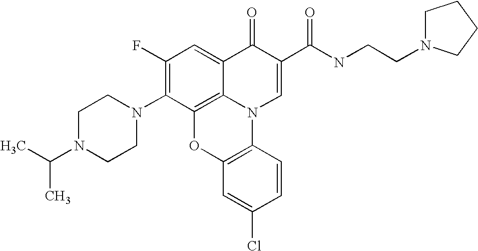

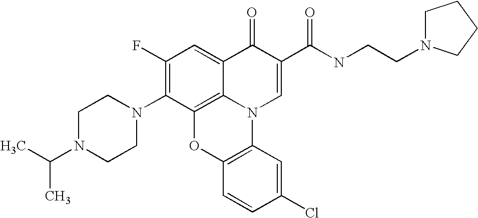

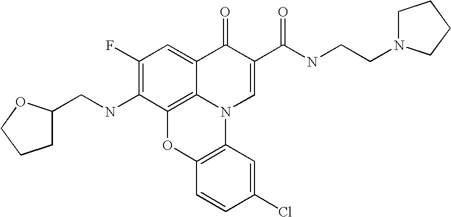

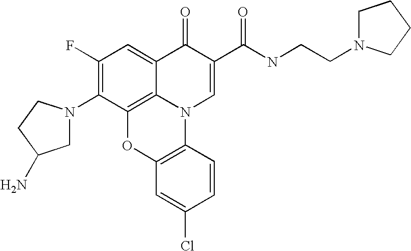

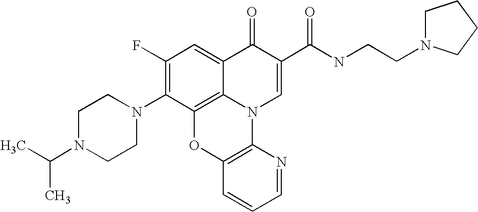

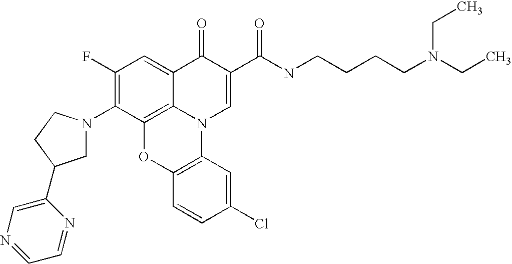

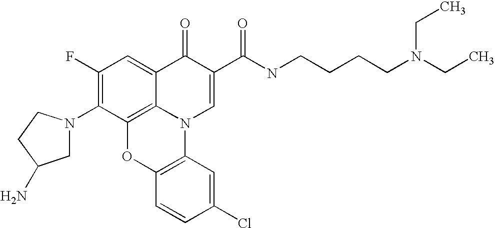

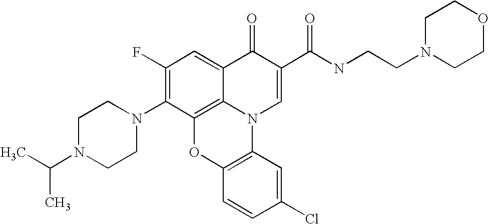

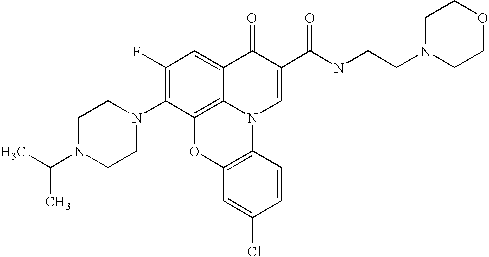

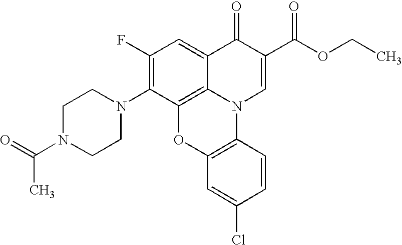

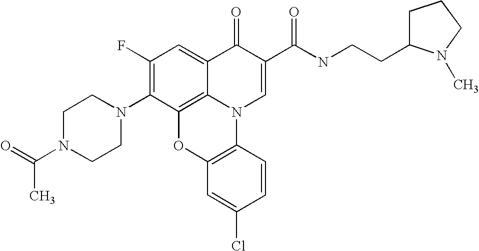

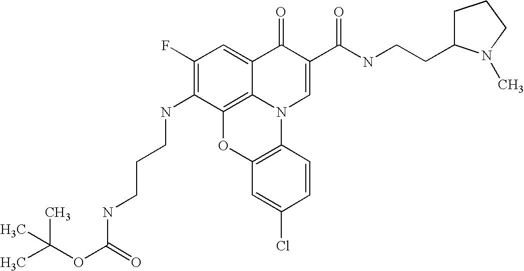

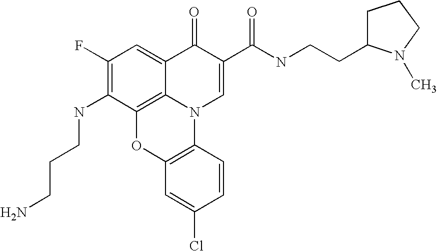

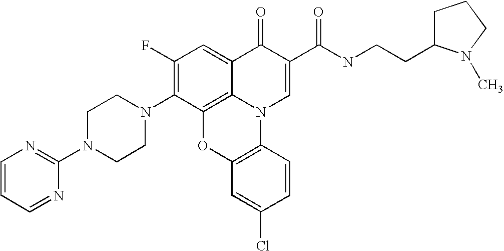

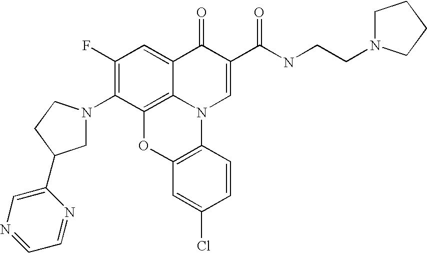

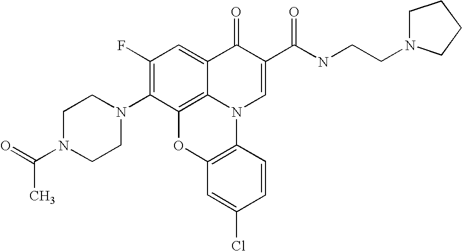

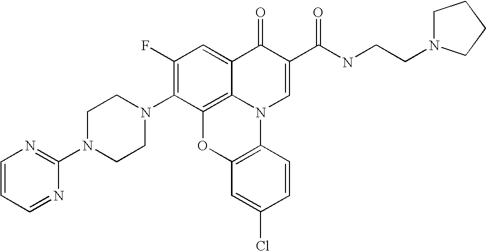

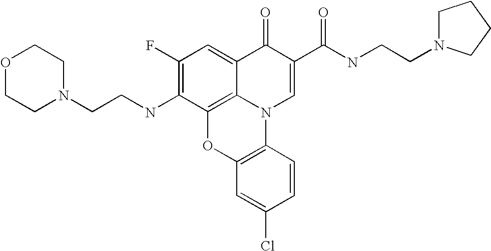

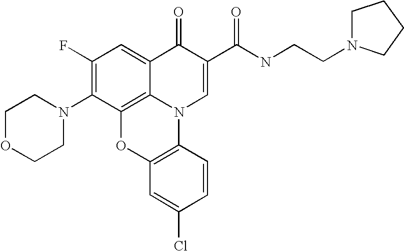

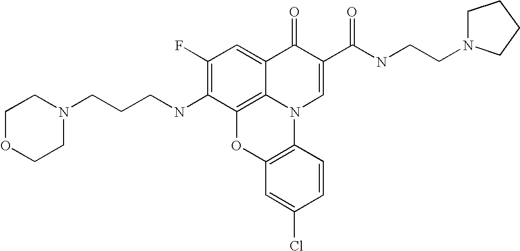

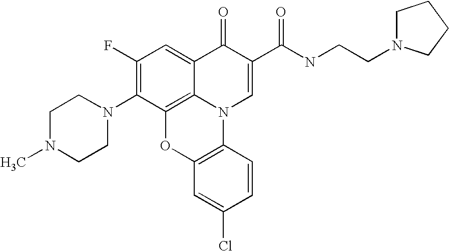

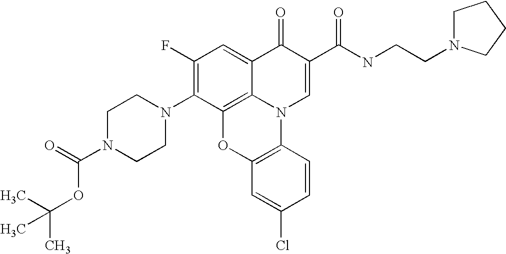

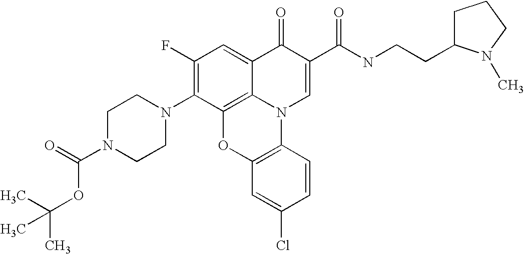

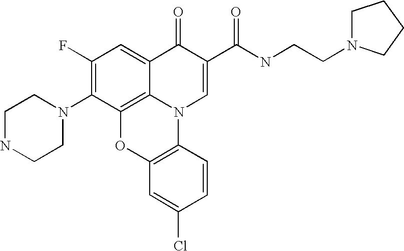

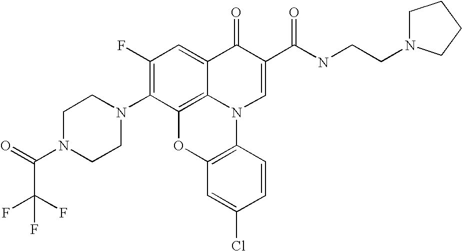

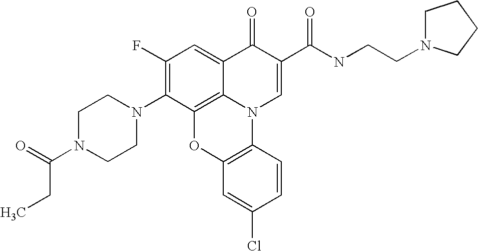

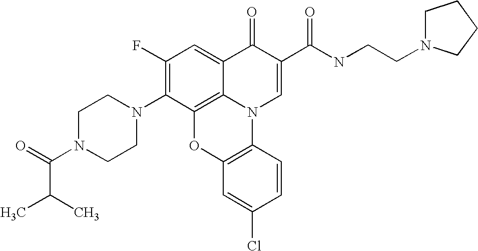

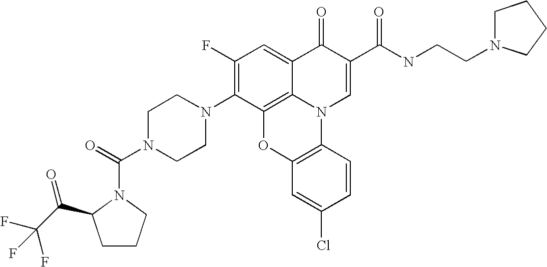

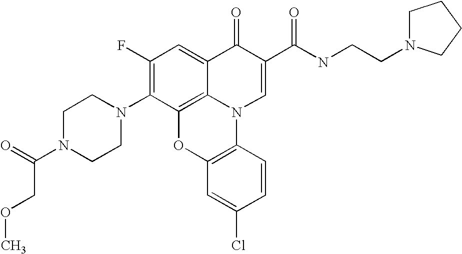

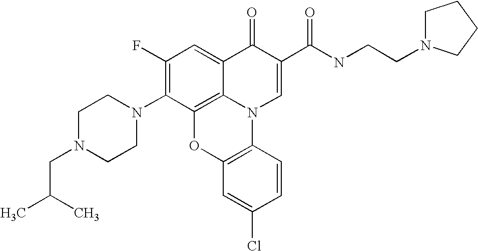

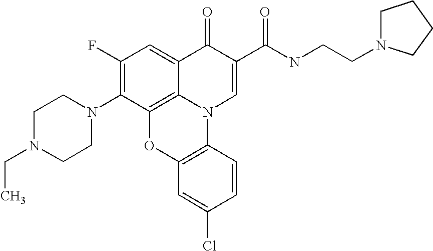

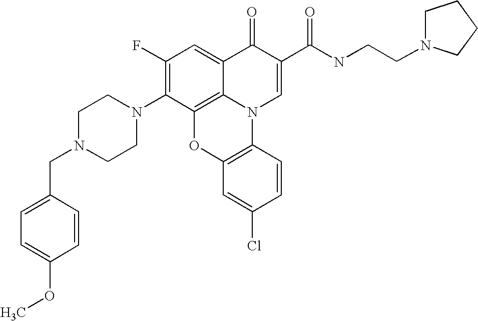

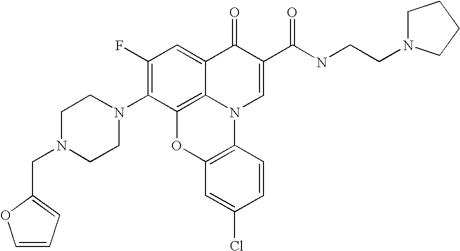

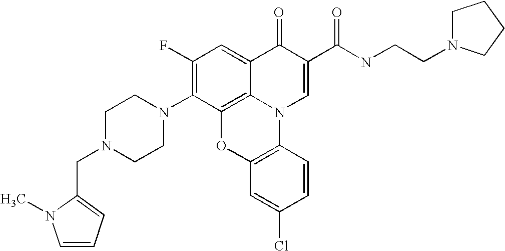

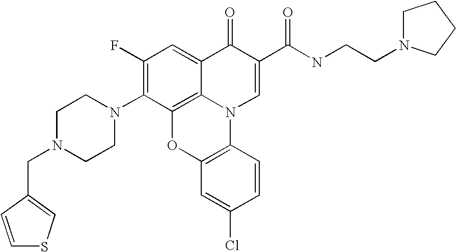

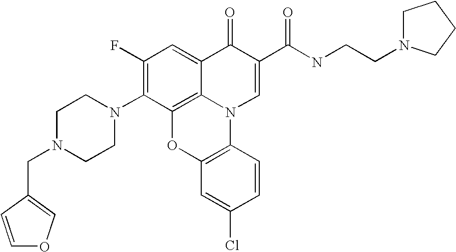

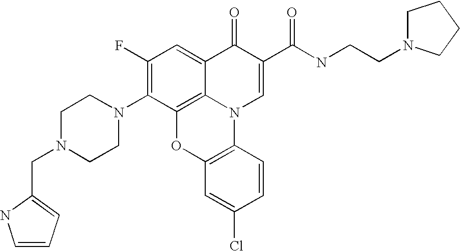

TABLE 4

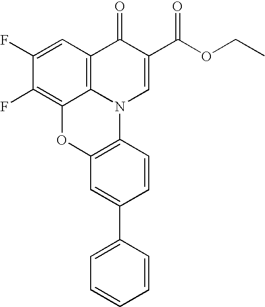

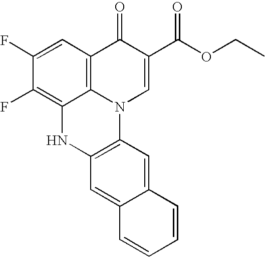

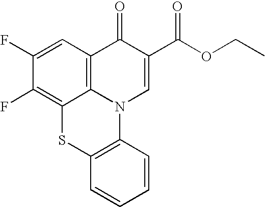

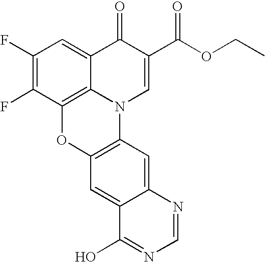

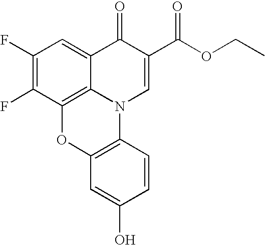

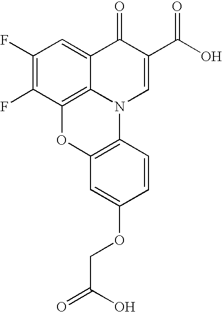

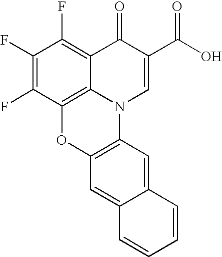

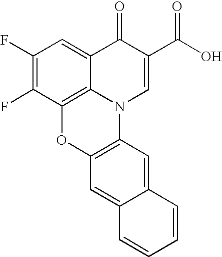

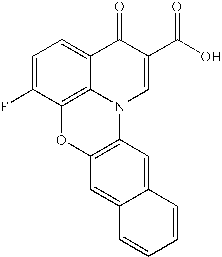

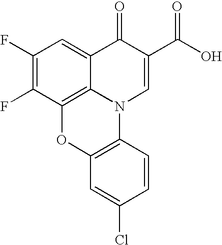

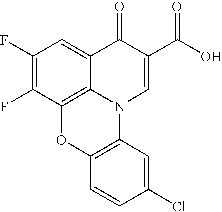

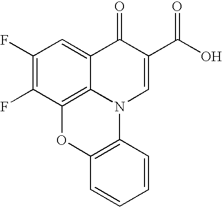

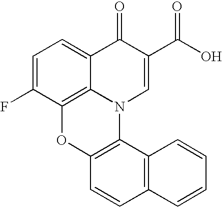

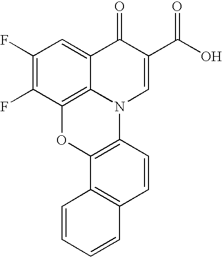

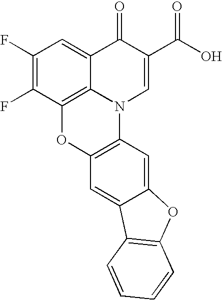

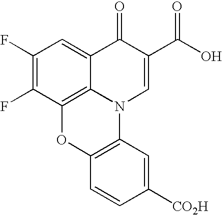

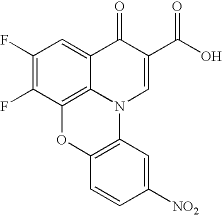

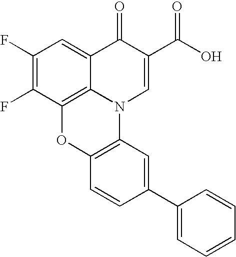

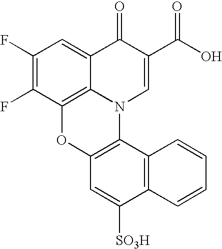

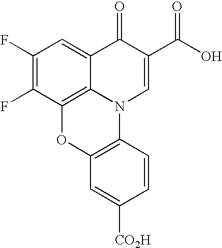

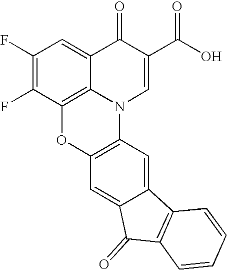

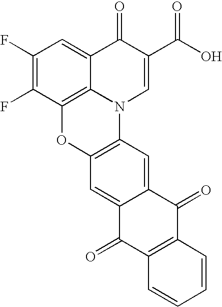

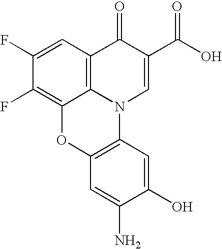

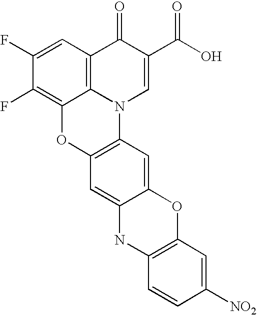

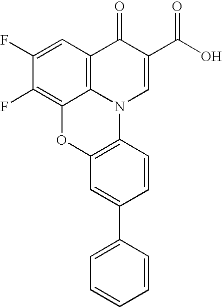

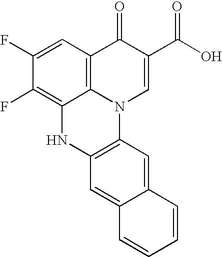

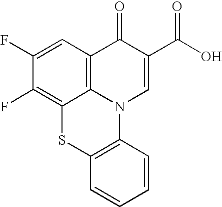

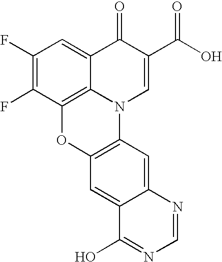

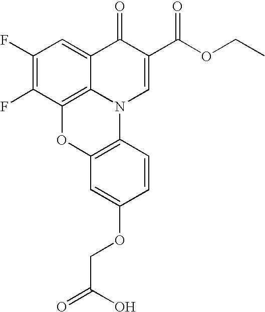

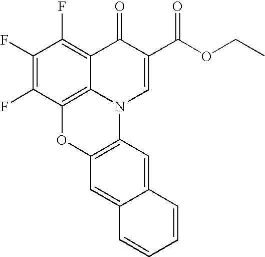

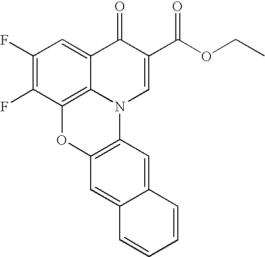

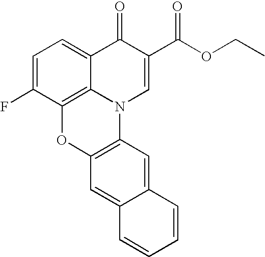

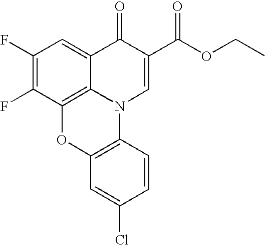

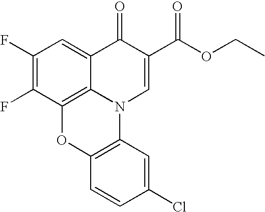

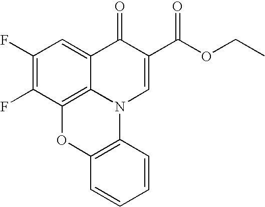

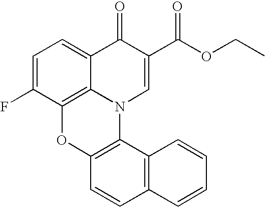

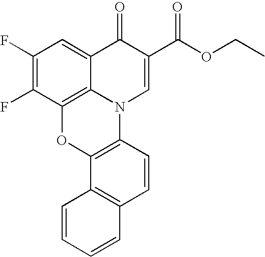

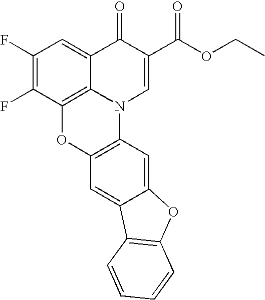

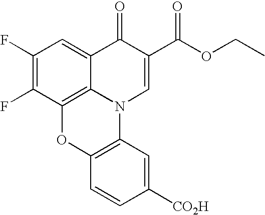

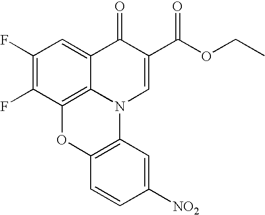

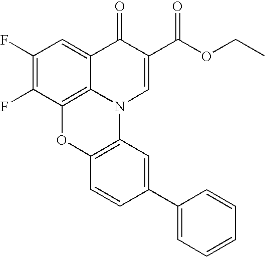

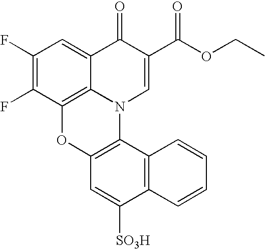

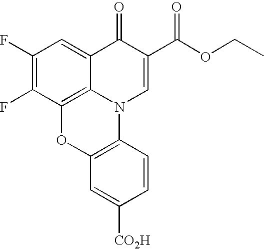

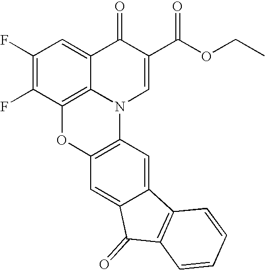

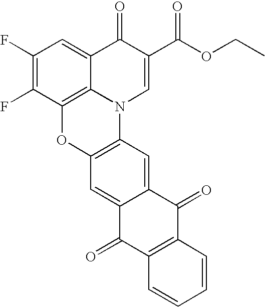

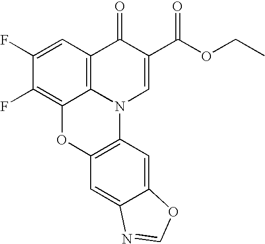

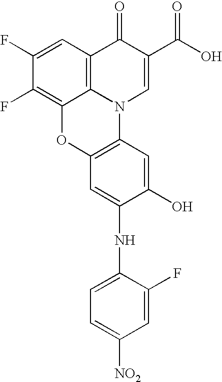

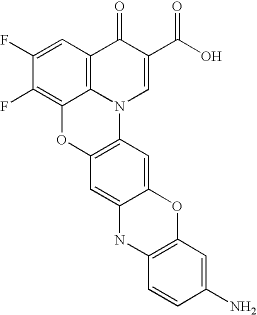

The person of ordinary skill in the art can select and prepare a nucleotide sequence interacting nucleic acid molecule. In certain embodiments, the interacting nucleic acid molecule contains a sequence complementary to a nucleotide sequence described herein, and is termed an “antisense” nucleic acid. Antisense nucleic acids may comprise or consist of analog or derivative nucleic acids, such as polyamide nucleic acids (PNA), locked nucleic acids (LNA) and other 2′ modified nucleic acids, and others exemplified in U.S. Pat. Nos. 4,469,863; 5,536,821; 5,541,306; 5,637,683; 5,637,684; 5,700,922; 5,717,083; 5,719,262; 5,739,308; 5,773,601; 5,886,165; 5,929,226; 5,977,296; 6,140,482; 5,614,622; 5,739,314; 5,955,599; 5,962,674; 6,117,992; WIPO publications WO 00/56746, WO 00/75372 and WO 01/14398, and related publications. An antisense nucleic acid sometimes is designed, prepared and/or utilized by the artisan to inhibit a nucleic acid. The antisense nucleic acid can be complementary to an entire coding strand, or to a portion thereof or a substantially identical sequence thereof. In another embodiment, the antisense nucleic acid molecule is antisense to a “noncoding region” of the coding strand of a nucleotide sequence. An antisense nucleic acid can be complementary to the entire coding region of a nucleotide sequence, and often the antisense nucleic acid is an oligonucleotide antisense to only a portion of a coding or noncoding region of the nucleotide sequence. For example, the antisense oligonucleotide can be complementary to the region surrounding the translation start site of the mRNA, e.g., between the −10 and +10 regions of the target gene nucleotide sequence of interest. An antisense oligonucleotide can be, for example, about 7, 10, 15, 20, 25, 30, 35, 40, 45, 50, 55, 60, 65, 70, 75, 80, or more nucleotides in length.

An antisense nucleic acid can be constructed using standard chemical synthesis or enzymic ligation reactions. For example, an antisense nucleic acid (e.g., an antisense oligonucleotide) can be chemically synthesized using naturally occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used). Antisense nucleic acid also can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

When utilized in animals, antisense nucleic acids typically are administered to a subject (e.g., by direct injection at a tissue site or intravenous administration) or generated in situ such that they hybridize with or bind to cellular mRNA and/or genomic DNA encoding a polypeptide and thereby inhibit expression of the polypeptide, for example, by inhibiting transcription and/or translation. Alternatively, antisense nucleic acid molecules can be modified to target selected cells and then are administered systemically. For systemic administration, antisense molecules can be modified such that they specifically bind to receptors or antigens expressed on a selected cell surface, for example, by linking antisense nucleic acid molecules to peptides or antibodies which bind to cell surface receptors or antigens. Antisense nucleic acid molecules can also be delivered to cells using the vectors described herein. Sufficient intracellular concentrations of antisense molecules are achieved by incorporating a strong promoter, such as a CMV promoter, pol II promoter or pol III promoter, in the vector construct.

Antisense nucleic acid molecules sometimes are alpha-anomeric nucleic acid molecules. An alpha-anomeric nucleic acid molecule forms specific double-stranded hybrids with complementary RNA in which, contrary to the usual beta-units, the strands run parallel to each other (Gaultier et al., Nucleic Acids. Res. 15: 6625-6641 (1987)). Antisense nucleic acid molecules also can comprise a 2′-o-methylribonucleotide (Inoue et al., Nucleic Acids Res. 15: 6131-6148 (1987)) or a chimeric RNA-DNA analogue (Inoue et al., FEBS Lett. 215: 327-330 (1987)). Antisense nucleic acids sometimes are composed of DNA or PNA or any other nucleic acid derivatives described previously.

An antisense nucleic acid is a ribozyme in some embodiments. A ribozyme having specificity for a nucleotide sequence can include one or more sequences complementary to such a nucleotide sequence, and a sequence having a known catalytic region responsible for mRNA cleavage (e.g., U.S. Pat. No. 5,093,246 or Haselhoff and Gerlach, Nature 334: 585-591 (1988)). For example, a derivative of a Tetrahymena L-19 IVS RNA is sometimes utilized in which the nucleotide sequence of the active site is complementary to the nucleotide sequence to be cleaved in a mRNA (e.g., Cech et al. U.S. Pat. No. 4,987,071; and Cech et al. U.S. Pat. No. 5,116,742). Nucleotide sequences also may be utilized to select a catalytic RNA having a specific ribonuclease activity from a pool of RNA molecules (e.g., Bartel & Szostak, Science 261: 1411-1418 (1993)).

Specific binding reagents sometimes are nucleic acids that can form triple helix structures with a nucleotide sequence. Triple helix formation can be enhanced by generating a “switchback” nucleic acid molecule. Switchback molecules are synthesized in an alternating 5′-3′,3′-5′ manner, such that they base pair with first one strand of a duplex and then the other, eliminating the necessity for a sizeable stretch of purines or pyrimidines being present on one strand of a duplex.

An artisan may select an interfering RNA (RNAi) or siRNA nucleotide sequence interacting agent for use. The nucleic acid selected sometimes is the RNAi or siRNA or a nucleic acid that encodes such products. The term “RNAi” as used herein refers to double-stranded RNA (dsRNA) which mediates degradation of specific mRNAs, and can also be used to lower or eliminate gene expression. The term “short interfering nucleic acid”, “siNA”, “short interfering RNA”, “siRNA”, “short interfering nucleic acid molecule”, “short interfering oligonucleotide molecule”, or “chemically-modified short interfering nucleic acid molecule” as used herein refers to any nucleic acid molecule directed against a gene. For example, a siRNA is capable of inhibiting or down regulating gene expression or viral replication, for example by mediating RNA interference “RNAi” or gene silencing in a sequence-specific manner; see for example Zamore et al., 2000, Cell, 101, 25-33; Bass, 2001, Nature, 411, 428-429; Elbashir et al., 2001, Nature, 411, 494-498; and Kreutzer et al., International PCT Publication No. WO 00/44895; Zernicka-Goetz et al., International PCT Publication No. WO 01/36646; Fire, International PCT Publication No. WO 99/32619; Plaetinck et al., International PCT Publication No. WO 00/01846; Mello and Fire, International PCT Publication No. WO 01/29058; Deschamps-Depaillette, International PCT Publication No. WO 99/07409; and Li et al., International PCT Publication No. WO 00/44914; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237; Hutvagner and Zamore, 2002, Science, 297, 2056-60; McManus et al., 2002, RNA, 8, 842-850; Reinhart et al., 2002, Gene & Dev., 16, 1616-1626; and Reinhart & Bartel, 2002, Science, 297, 1831). There is no particular limitation in the length of siRNA as long as it does not show toxicity. Examples of modified RNAi and siRNA include STEALTH™ forms (Invitrogen Corp., Carlsbad, Calif.), forms described in U.S. Patent Publication No. 2004/0014956 (application Ser. No. 10/357,529) and U.S. patent application Ser. No. 11/049,636, filed Feb. 2, 2005), shRNA, MIRs and other forms described hereafter.

A siNA can be a double-stranded polynucleotide molecule comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof. The siNA can be assembled from two separate oligonucleotides, where one strand is the sense strand and the other is the antisense strand, wherein the antisense and sense strands are self-complementary (i.e. each strand comprises nucleotide sequence that is complementary to nucleotide sequence in the other strand; such as where the antisense strand and sense strand form a duplex or double stranded structure, for example wherein the double stranded region is about 19 base pairs); the antisense strand comprises nucleotide sequence that is complementary to nucleotide sequence in a target nucleic acid molecule or a portion thereof and the sense strand comprises nucleotide sequence corresponding to the target nucleic acid sequence or a portion thereof. Alternatively, the siNA is assembled from a single oligonucleotide, where the self-complementary sense and antisense regions of the siNA are linked by means of a nucleic acid based or non-nucleic acid-based linker(s). The siNA can be a polynucleotide with a duplex, asymmetric duplex, hairpin or asymmetric hairpin secondary structure, having self-complementary sense and antisense regions, wherein the antisense region comprises nucleotide sequence that is complementary to nucleotide sequence in a separate target nucleic acid molecule or a portion thereof and the sense region having nucleotide sequence corresponding to the target nucleic acid sequence or a portion thereof. The siNA can be a circular single-stranded polynucleotide having two or more loop structures and a stem comprising self-complementary sense and antisense regions, wherein the antisense region comprises nucleotide sequence that is complementary to nucleotide sequence in a target nucleic acid molecule or a portion thereof and the sense region having nucleotide sequence corresponding to the target nucleic acid sequence or a portion thereof, and wherein the circular polynucleotide can be processed either in vivo or in vitro to generate an active siNA molecule capable of mediating RNAi. The siNA can also comprise a single stranded polynucleotide having nucleotide sequence complementary to nucleotide sequence in a target nucleic acid molecule or a portion thereof (for example, where such siNA molecule does not require the presence within the siNA molecule of nucleotide sequence corresponding to the target nucleic acid sequence or a portion thereof), wherein the single stranded polynucleotide can further comprise a terminal phosphate group, such as a 5′-phosphate (see for example Martinez et al., 2002, Cell., 110, 563-574 and Schwarz et al., 2002, Molecular Cell, 10, 537-568), or 5′,3′-diphosphate. In certain embodiments, the siNA molecule of the invention comprises separate sense and antisense sequences or regions, wherein the sense and antisense regions are covalently linked by nucleotide or non-nucleotide linkers molecules as is known in the art, or are alternately non-covalently linked by ionic interactions, hydrogen bonding, van der waals interactions, hydrophobic interactions, and/or stacking interactions. In certain embodiments, the siNA molecules of the invention comprise nucleotide sequence that is complementary to nucleotide sequence of a target gene. In another embodiment, the siNA molecule of the invention interacts with nucleotide sequence of a target gene in a malmer that causes inhibition of expression of the target gene.

The double-stranded RNA portions of siRNAs in which two RNA strands pair are not limited to the completely paired forms, and may contain non-pairing portions due to mismatch (the corresponding nucleotides are not complementary), bulge (lacking in the corresponding complementary nucleotide on one strand), and the like. Non-pairing portions can be contained to the extent that they do not interfere with siRNA formation. The “bulge” used herein preferably comprise 1 to 2 non-pairing nucleotides, and the double-stranded RNA region of siRNAs in which two RNA strands pair up contains preferably 1 to 7, more preferably 1 to 5 bulges. In addition, the “mismatch” used herein is contained in the double-stranded RNA region of siRNAs in which two RNA strands pair up, preferably 1 to 7, more preferably 1 to 5, in number. In a preferable mismatch, one of the nucleotides is guanine, and the other is uracil. Such a mismatch is due to a mutation from C to T, G to A, or mixtures thereof in DNA coding for sense RNA, but not particularly limited to them. Furthermore, in the present invention, the double-stranded RNA region of siRNAs in which two RNA strands pair up may contain both bulge and mismatched, which sum up to, preferably 1 to 7, more preferably 1 to 5 in number. The terminal structure of siRNA may be either blunt or cohesive (overhanging) as long as siRNA enables to silence the target gene expression due to its RNAi effect.

As used herein, siRNA molecules need not be limited to those molecules containing only RNA, but further encompasses chemically-modified nucleotides and non-nucleotides. In addition, as used herein, the term RNAi is meant to be equivalent to other terms used to describe sequence specific RNA interference, such as post transcriptional gene silencing, translational inhibition, or epigenetics. For example, siRNA molecules of the invention can be used to epigenetically silence genes at both the post-transcriptional level or the pre-transcriptional level. In a non-limiting example, epigenetic regulation of gene expression by siRNA molecules of the invention can result from siRNA mediated modification of cliromatin structure to alter gene expression (see, for example, Verdel et al., 2004, Science, 303, 672-676; Pal-Bhadra et al., 2004, Science, 303, 669-672; Allshire, 2002, Science, 297, 1818-1819; Volpe et al., 2002, Science, 297, 1833-1837; Jenuwein, 2002, Science, 297, 2215-2218; and Hall et al., 2002, Science, 297, 2232-2237).

RNAi may be designed by those methods known to those of ordinary skill in the art. In one example, siRNA may be designed by classifying RNAi sequences, for example 1000 sequences, based on functionality, with a functional group being classified as having greater than 85% knockdown activity and a non-functional group with less than 85% knockdown activity. The distribution of base composition was calculated for entire the entire RNAi target sequence for both the functional group and the non-functional group. The ratio of base distribution of functional and non-functional group may then be used to build a score matrix for each position of RNAi sequence. For a given target sequence, the base for each position is scored, and then the log ratio of the multiplication of all the positions is taken as a final score. Using this score system, a very strong correlation may be found of the functional knockdown activity and the log ratio score. Once the target sequence is selected, it may be filtered through both fast NCBI blast and slow Smith Waterman algorithm search against the Unigene database to identify the gene-specific RNAi or siRNA. Sequences with at least one mismatch in the last 12 bases may be selected.

Nucleic acid reagents include those which are engineered, for example, to produce dsRNAs. Examples of such nucleic acid molecules include those with a sequence that, when transcribed, folds back upon itself to generate a hairpin molecule containing a double-stranded portion. One strand of the double-stranded portion may correspond to all or a portion of the sense strand of the mRNA transcribed from the gene to be silenced while the other strand of the double-stranded portion may correspond to all or a portion of the antisense strand. Other methods of producing dsRNAs may be used, for example, nucleic acid molecules may be engineered to have a first sequence that, when transcribed, corresponds to all or a portion of the sense strand of the in RNA transcribed from the gene to be silenced and a second sequence that, when transcribed, corresponds to all or portion of an antisense strand (i.e., the reverse complement) of the mRNA transcribed from the gene to be silenced.

Nucleic acid molecules which mediate RNAi may also be produced ex vivo, for example, by oligonucleotide synthesis. Oligonucleotide synthesis may be used for example, to design dsRNA molecules, as well as other nucleic acid molecules (e.g., other nucleic acid molecules which mediate RNAi) with one or more chemical modification (e.g., chemical modifications not commonly found in nucleic acid molecules such as the inclusion of 2′-O-methyl, 2′-O-ethyl, 2′-methoxyethoxy, 2′-O-propyl, 2′-fluoro, etc. groups).

In some embodiments, a dsRNA to be used to silence a gene may have one or more (e.g., one, two, three, four, five, six, etc.) regions of sequence homology or identity to a gene to be silenced. Regions of homology or identity may be from about 20 bp (base pairs) to about 5 kbp (kilo base pairs) in length, 20 bp to about 4 kbp in length, 20 bp to about 3 kbp in length, 20 bp to about 2.5 kbp in length, from about 20 bp to about 2 kbp in length, 20 bp to about 1.5 kbp in length, from about 20 bp to about 1 kbp in length, 20 bp to about 750 bp in length, from about 20 bp to about 500 bp in length, 20 bp to about 400 bp in length, 20 bp to about 300 bp in length, 20 bp to about 250 bp in length, from about 20 bp to about 200 bp in length, from about 20 bp to about 150 bp in length, from about 20 bp to about 100 bp in length, from about 20 bp to about 90 bp in length, from about 20 bp to about 80 bp in length, from about 20 bp to about 70 bp in length, from about 20 bp to about 60 bp in length, from about 20 bp to about 50 bp in length, from about 20 bp to about 40 bp in length, from about 20 bp to about 30 bp in length, from about 20 bp to about 25 bp in length, from about 15 bp to about 25 bp in length, from about 17 bp to about 25 bp in length, from about 19 bp to about 25 bp in length, from about 19 bp to about 23 bp in length, or from about 19 bp to about 21 bp in length.

A hairpin containing molecule having a double-stranded region may be used as RNAi. The length of the double stranded region may be from about 20 bp (base pairs) to about 2.5 kbp (kilo base pairs) in length, from about 20 bp to about 2 kbp in length, 20 bp to about 1.5 kbp in length, from about 20 bp to about 1 kbp in length, 20 bp to about 750 bp in length, from about 20 bp to about 500 bp in length, 20 bp to about 400 bp in length, 20 bp to about 300 bp in length, 20 bp to about 250 bp in length, from about 20 bp to about 200 bp in length, from about 20 bp to about 150 bp in length, from about 20 bp to about 100 bp in length, 20 bp to about 90 bp in length, 20 bp to about 80 bp in length, 20 bp to about 70 bp in length, 20 bp to about 60 bp in length, 20 bp to about 50 bp in length, 20 bp to about 40 bp in length, 20 bp to about 30 bp in length, or from about 20 bp to about 25 bp in length. The non-base-paired portion of the hairpin (i.e., loop) can be of any length that permits the two regions of homology that make up the double-stranded portion of the hairpin to fold back upon one another.

Any suitable promoter may be used to control the production of RNA from the nucleic acid reagent, such as a promoter described above. Promoters may be those recognized by any polymerase enzyme. For example, promoters may be promoters for RNA polymerase II or RNA polymerase III (e.g., a U6 promoter, an H1 promoter, etc.). Other suitable promoters include, but are not limited to, T7 promoter, cytomegalovirus (CMV) promoter, mouse mammary tumor virus (MMTV) promoter, metalothionine, RSV (Rous sarcoma virus) long terminal repeat, SV40 promoter, human growth hormone (hGH) promoter. Other suitable promoters are known to those skilled in the art and are within the scope of the present invention.

Double-stranded RNAs used in the practice of the invention may vary greatly in size. Further the size of the dsRNAs used will often depend on the cell type contacted with the dsRNA. As an example, animal cells such as those of C. elegans and Drosophila melanogaster do not generally undergo apoptosis when contacted with dsRNAs greater than about 30 nucleotides in length (i.e., 30 nucleotides of double stranded region) while mammalian cells typically do undergo apoptosis when exposed to such dsRNAs. Thus, the design of the particular experiment will often determine the size of dsRNAs employed.

In many instances, the double stranded region of dsRNAs contained within or encoded by nucleic acid molecules used in the practice of the invention will be within the following ranges: from about 20 to about 30 nucleotides, from about 20 to about 40 nucleotides, from about 20 to about 50 nucleotides, from about 20 to about 100 nucleotides, from about 22 to about 30 nucleotides, from about 22 to about 40 nucleotides, from about 20 to about 28 nucleotides, from about 22 to about 28 nucleotides, from about 25 to about 30 nucleotides, from about 25 to about 28 nucleotides, from about 30 to about 100 nucleotides, from about 30 to about 200 nucleotides, from about 30 to about 1,000 nucleotides, from about 30 to about 2,000 nucleotides, from about 50 to about 100 nucleotides, from about 50 to about 1,000 nucleotides, or from about 50 to about 2,000 nucleotides. The ranges above refer to the number of nucleotides present in double stranded regions. Thus, these ranges do not reflect the total length of the dsRNAs themselves. As an example, a blunt ended dsRNA formed from a single transcript of 50 nucleotides in total length with a 6 nucleotide loop, will have a double stranded region of 23 nucleotides.

As suggested above, dsRNAs used in the practice of the invention may be blunt ended, may have one blunt end, or may have overhangs on both ends. Further, when one or more overhang is present, the overhang(s) may be on the 3′ and/or 5′ strands at one or both ends. Additionally, these overhangs may independently be of any length (e.g., one, two, three, four, five, etc. nucleotides). As an example, STEALTH™ RNAi is blunt at both ends.

Also included are sets of RNAi and those which generate RNAi. Such sets include those which either (1) are designed to produce or (2) contain more than one dsRNA directed against the same target gene. As an example, the invention includes sets of STEALTH™ RNAi wherein more than one STEALTH™ RNAi shares sequence homology or identity to different regions of the same target gene.

An antibody or antibody fragment can be generated by and used by the artisan as a nucleotide sequence interacting agent. Antibodies sometimes are IgG, IgM, IgA, IgE, or an isotype thereof (e.g., IgG1, IgG2a, IgG2b or IgG3), sometimes are polyclonal or monoclonal, and sometimes are chimeric, humanized or bispecific versions of such antibodies. Polyclonal and monoclonal antibodies that bind specific antigens are commercially available, and methods for generating such antibodies are known. In general, polyclonal antibodies are produced by injecting an isolated antigen (e.g., rDNA or rRNA subsequence described herein) into a suitable animal (e.g., a goat or rabbit); collecting blood and/or other tissues from the animal containing antibodies specific for the antigen and purifying the antibody. Methods for generating monoclonal antibodies, in general, include injecting an animal with an isolated antigen (e.g., often a mouse or a rat); isolating splenocytes from the animal; fusing the splenocytes with myeloma cells to form hybridomas; isolating the hybridomas and selecting hybridomas that produce monoclonal antibodies which specifically bind the antigen (e.g., Kohler & Milstein, Nature 256:495 497 (1975) and StGroth & Scheidegger, J Immunol Methods 5:1 21 (1980)).

Methods for generating chimeric and humanized antibodies also are known (see, e.g., U.S. Pat. No. 5,530,101 (Queen, et al.), U.S. Pat. No. 5,707,622 (Fung, et al.) and U.S. Pat. Nos. 5,994,524 and 6,245,894 (Matsushima, et al.)), which generally involve transplanting an antibody variable region from one species (e.g., mouse) into an antibody constant domain of another species (e.g., human). Antigen-binding regions of antibodies (e.g., Fab regions) include a light chain and a heavy chain, and the variable region is composed of regions from the light chain and the heavy chain. Given that the variable region of an antibody is formed from six complementarity-determining regions (CDRs) in the heavy and light chain variable regions, one or more CDRs from one antibody can be substituted (i.e., grafted) with a CDR of another antibody to generate chimeric antibodies. Also, humanized antibodies are generated by introducing amino acid substitutions that render the resulting antibody less immunogenic when administered to humans.

A specific binding reagent sometimes is an antibody fragment, such as a Fab, Fab′, F(ab)′2, Dab, Fv or single-chain Fv (ScFv) fragment, and methods for generating antibody fragments are known (see, e.g., U.S. Pat. Nos. 6,099,842 and 5,990,296 and PCT/GB00/04317). In some embodiments, a binding partner in one or more hybrids is a single-chain antibody fragment, which sometimes are constructed by joining a heavy chain variable region with a light chain variable region by a polypeptide linker (e.g., the linker is attached at the C-terminus or N-terminus of each chain) by recombinant molecular biology processes. Such fragments often exhibit specificities and affinities for an antigen similar to the original monoclonal antibodies. Bifunctional antibodies sometimes are constructed by engineering two different binding specificities into a single antibody chain and sometimes are constructed by joining two Fab′ regions together, where each Fab′ region is from a different antibody (e.g., U.S. Pat. No. 6,342,221). Antibody fragments often comprise engineered regions such as CDR-grafted or humanized fragments. In certain embodiments the binding partner is an intact immunoglobulin, and in other embodiments the binding partner is a Fab monomer or a Fab dimer.

### Compositions, Cells and Animals Comprising Nucleic Acids and/or Interacting Molecules

Provided also is a composition comprising a nucleic acid described herein. In certain embodiments, a composition comprises a nucleic acid that includes a nucleotide sequence complementary to a human DNA or RNA nucleotide sequence described herein. A composition may comprise a pharmaceutically acceptable carrier in some embodiments, and a composition sometimes comprises a nucleic acid and a compound that binds to a human nucleotide sequence in the nucleic acid (e.g., specifically binds to the nucleotide sequence). In certain embodiments, the compound is a quinolone analog, such as a compound described herein.

Also provided is a cell or animal comprising an isolated nucleic acid described herein. Any type of cell can be utilized, and sometimes the cell is a cell line maintained or proliferated in tissue culture. The isolated nucleic acid may be incorporated into one or more cells of an animal, such as a research animal (e.g., rodent (e.g., mouse, rat, guinea pig, hamster, rabbit), ungulate (e.g., bovine, porcine, equine, caprine), cat, dog, monkey or ape). Methods for inserting compounds and other molecules into cells are known to the person of ordinary skill in the art, such as in methods described hereafter.

A cell may over-express or under-express a nucleotide sequence described herein. A cell can be processed in a variety of manners. For example, an artisan may prepare a lysate from a cell reagent and optionally isolate or purify components of the cell, may transfect the cell with a nucleic acid reagent, may fix a cell reagent to a slide for analysis (e.g., microscopic analysis) and can immobilize a cell to a solid phase. A cell that “over-expresses” a nucleotide sequence produces at least two, three, four or five times or more of the product as compared to a native cell from an organism that has not been genetically modified and/or exhibits no apparent symptom of a cell-proliferative disorder. Over-expressing cells may be stably transfected or transiently transfected with a nucleic acid that encodes the nucleotide sequence. A cell that “under-expresses” a nucleotide sequence produces at least five times less of the product as compared to a native cell from an organism that has not been genetically modified and/or exhibits no apparent symptom of a cell-proliferative disorder. In some embodiments, a cell that under-expresses a nucleotide sequence contains no nucleic acid that can encode such a product (e.g., the cell is from a knock-out mouse) and no detectable amount of the product is produced. Methods for generating knock-out animals and using cells extracted therefrom are known (e.g., Miller et al., J. Cell. Biol. 165: 407-419 (2004)). A cell that under-expresses a nucleotide sequence, for example, sometimes is in contact with a nucleic acid inhibitor that blocks or reduces the amount of the product produced by the cell in the absence of the inhibitor. An over-expressing or under-expressing cell may be within an organism (in vivo) or from an organism (ex vivo or in vitro).

The artisan may select any cell for generating cell compositions of the invention (e.g., cells that over-express or under-express a nucleotide sequence). Cells include, but are not limited to, bacterial cells (e.g., Escherichia spp. cells (e.g., Expressway™ HTP Cell-Free E. coli Expression Kit, Invitrogen, California) such as DH10B, Stb12, DH5-alpha, DB3, DB3.1 for example), DB4, DB5, JDP682 and ccdA-over (e.g., U.S. application Ser. No. 09/518,188), Bacillus spp. cells (e.g., B. subtilis and B. megaterium cells), Streptomyces spp. cells, Erwinia spp. cells, Klebsiella spp. cells, Serratia spp. cells (particularly S. marcessans cells), Pseudomonas spp. cells (particularly P. aeruginosa cells), and Salmonella spp. cells (particularly S. typhimurium and S. typhi cells); photosynthetic bacteria (e.g., green non-sulfur bacteria (e.g., Choroflexus spp. (e.g., C. aurantiacus), Chloronema spp. (e.g., C. gigateum)), green sulfur bacteria (e.g., Chlorobium spp. (e.g., C. limicola), Pelodictyon spp. (e.g., P. luteolum), purple sulfur bacteria (e.g., Clromatium spp. (e.g., C. okenii)), and purple non-sulfur bacteria (e.g., Rhodospirillum spp. (e.g., R. rubrum), Rhodobacter spp. (e.g., R. sphaeroides, R. capsulatus), Rhodomicrobium spp. (e.g., R. vanellii)); yeast cells (e.g., Saccharomyces cerevisiae cells and Pichia pastoris cells); insect cells (e.g., Drosophila (e.g., Drosophila melanogaster), Spodoptera (e.g., Spodoptera frugiperda Sf9 and Sf21 cells) and Trichoplusa (e.g., High-Five cells); nematode cells (e.g., C. elegans cells); avian cells; amphibian cells (e.g., Xenopus laevis cells); reptilian cells; and mammalian cells (e.g., NIH3T3, 293, CHO, COS, VERO, C127, BHK, Per-C6, Bowes melanoma and HeLa cells). In specific embodiments, cells are pancreatic cells, colorectal cells, renal cells or Burkitt's lymphoma cells. In some embodiments, pancreatic cell lines such as PC3, HCT116, HT29, MIA Paca-2, HPAC, Hs700T, Panc10.05, Panc 02.13, PL45, SW 190, Hs 766T, CFPAC-1 and PANC-1 are utilized. These and other suitable cells are available commercially, for example, from Invitrogen Corporation, (Carlsbad, Calif.), American Type Culture Collection (Manassas, Va.), and Agricultural Research Culture Collection (NRRL; Peoria, Ill.).

Use of Nucleotide Sequences and Interacting Molecules

Nucleotide sequence interacting molecules sometimes are utilized to effect a cellular response, and are utilized to effect a therapeutic response in some embodiments. Accordingly, provided herein is a method for inhibiting RNA synthesis in cells, which comprises contacting cells with a compound that interacts with a nucleotide sequence described herein in an amount effective to reduce rRNA synthesis in cells. Such methods may be conducted in vitro, in vivo and/or ex vivo. Accordingly, some in vivo and ex vivo embodiments are directed to a method for inhibiting RNA synthesis in cells of a subject, which comprises administering a compound that interacts with a nucleotide sequence described herein to a subject in need thereof in an amount effective to reduce RNA synthesis in cells of the subject. In certain embodiments, polymerase II-directed RNA synthesis is reduced. In some embodiments, cells can be contacted with one or more compounds, one or more of which interact with a nucleotide sequence described herein (e.g., one drug or drug and co-drug(s) methodologies). In certain embodiments, a compound is a quinolone derivative, such as a quinolone derivative described herein. The cells often are cancer cells, such as cells undergoing higher than normal proliferation and tumor cells, for example.

In some embodiments, cells are contacted with a compound that interacts with a nucleotide sequence described herein in combination with one or more other therapies (e.g., tumor removal surgery and/or radiation therapy) and/or other molecules (e.g., co-drugs) that exert other effects in cells. For example, a co-drug may be selected that reduces cell proliferation or reduces tissue inflammation. The person of ordinary skill in the art may select and administer a wide variety of co-drugs in a combination approach. Non-limiting examples of co-drugs include avastin, dacarbazine (e.g., multiple myeloma), 5-fluorouracil (e.g., pancreatic cancer), gemcitabine (e.g., pancreatic cancer), and gleevac (e.g., CML).

The term “inhibiting RNA synthesis” as used herein refers to reducing the amount of RNA produced by a cell after a cell is contacted with the compound or after a compound is administered to a subject. In certain embodiments, polymerase II-directed RNA synthesis is reduced. In some embodiments, RNA levels are reduced by about 10%, about 15%, about 20%, about 25%, about 30%, about 40%, about 45%, about 50%, about 55%, about 60%, about 70%, about 75%, about 80%, about 90%, or about 95% or more in a specific time frame, such as about 1 hour, about 2 hours, about 3 hours, about 4 hours, about 5 hours, about 6 hours, about 7 hours, about 8 hours, about 9 hours, about 10 hours, about 12 hours, about 16 hours, about 20 hours, or about 24 hours in particular cells after cells are contacted with the compound or the compound is administered to a subject. Particular cells in which RNA levels are reduced sometimes are cancer cells or cells undergoing proliferation at greater rates than other cells in a system. Levels of RNA in a cell can be determined in vitro and in vivo (e.g., see Examples section).

The term “interacting with a nucleotide sequence” as used herein refers to a direct interaction or indirect interaction of a compound with the nucleotide sequence. In some embodiments, a compound may directly bind to an RNA nucleotide sequence described herein. A compound may directly bind to a DNA nucleotide sequence described herein. In certain embodiments, a compound may bind to and/or stabilize a quadruplex structure in RNA or DNA. In some embodiments, a compound may directly bind to a protein that binds to or interacts with a RNA or DNA nucleotide sequence, such as a protein involved in RNA synthesis, a protein involved in RNA elongation (e.g., a polymerase such as Pol II or a transcription protein effector), or a protein involved in pre-RNA processing (e.g., an endonuclease, exonuclease, RNA helicase), for example.

In certain embodiments, provided also is a method for effecting a cellular response by contacting a cell with a compound that binds to a human nucleotide sequence and/or structure described herein. The cellular response sometimes is (a) substantial phosphorylation of H2AX, p53, chk1 and p38 MAPK proteins; (b) redistribution of nucleolin from nucleoli into the nucleoplasm; (c) release of cathepsin D from lysosomes; (d) induction of apoptosis; (e) induction of chromosomal laddering; (f) induction of apoptosis without substantially arresting cell cycle progression; and/or (g) induction of apoptosis and inducing cell cycle arrest (e.g., S-phase and/or G1 arrest).

The term “substantial phosphorylation” as used herein, refers to one or more sites of a particular type of protein or fragment linked to a phosphate moiety. In certain embodiments, phosphorylation is substantial when it is detectable, and in some embodiments, phosphorylation is substantial when about 55% to 99% of the particular type of protein or fragment is phosphorylated or phosphorylated at a particular site. Particular proteins sometimes are H2AX, DNA-PK, p53, chk1, JNK and p38 MAPK proteins or fragments thereof that contain one or more phosphorylation sites. Methods for detecting phosphorylation of such proteins are described herein.

The term “apoptosis” as used herein refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages. Chromosomal DNA often is cleaved in cells undergoing apoptosis such that a ladder is visualized when cellular DNA is analyzed by gel electrophoresis. Apoptosis sometimes is monitored by detecting caspase activity, such as caspase S activity, and by monitoring phosphatidyl serine translocation. Methods described herein are designed to preferentially induce apoptosis of cancer cells, such as cancer cells in tumors, over non-cancerous cells.

The term “cell cycle progression” as used herein refers to the process in which a cell divides and proliferates. Particular phases of cell cycle progression are recognized, such as the mitosis and interphase. There are sub-phases within interphase, such as G1, S and G2 phases, and sub-phases within mitosis, such as prophase, metaphase, anaphase, telophase and cytokinesis. Cell cycle progression sometimes is substantially arrested in a particular phase of the cell cycle (e.g., about 90% of cells in a population are arrested at a particular phase, such as G1 or S phase). In some embodiments, cell cycle progression sometimes is not arrested significantly in any one phase of the cycle. For example, a subpopulation of cells can be substantially arrested in the S-phase of the cell cycle and another subpopulation of cells can be substantially arrested at the G1 phase of the cell cycle. In certain embodiments, the cell cycle is not arrested substantially at any particular phase of the cell cycle. Arrest determinations often are performed at one or more specific time points, such as about 4 hours, about 8 hours, about 12 hours, about 16 hours, about 20 hours, about 24 hours, about 36 hours or about 48 hours, and apoptosis may have occurred or may be occurring during or by these time points.

The term “redistribution of nucleolin” refers to migration of the protein nucleolin or a fragment thereof from the nucleolus to another portion of a cell, such as the nucleoplasm. Different types of nucleolin exist and are described herein. Nucleolin sometimes is distributed from the nucleolus when detectable levels of nucleolin are present in another cell compartment (e.g., the nucleolus). Methods for detecting nucleolin are known and described herein. A nucleolus of cells in which nucleolin is redistributed may include about 55% to about 95% of the nucleolin in untreated cells in some embodiments. A nucleolus of cells in which nucleolin is substantially redistributed may include about 5% to about 50% of the nucleolin in untreated cells.

A candidate molecule or nucleic acid may be prepared as a formulation or medicament and may be used as a therapeutic. In some embodiments, provided is a method for treating a disorder, comprising administering a molecule identified by a method described herein to a subject in an amount effective to treat the disorder, whereby administration of the molecule treats the disorder. The terms “treating,” “treatment” and “therapeutic effect” as used herein refer to ameliorating, alleviating, lessening, and removing symptoms of a disease or condition. In some embodiments involving a nucleic acid candidate molecule, such as in gene therapies, antisense therapies, and siRNA or RNAi therapies, the nucleic acid may integrate with a host genome or not integrate. Any suitable formulation of a candidate molecule can be prepared for administration. Any suitable route of administration may be used, including but not limited to oral, parenteral, intravenous, intramuscular, transdermal, topical and subcutaneous routes. The subject may be a rodent (e.g., mouse, rat, hamster, guinea pig, rabbit), ungulate (e.g., bovine, porcine, equine, caprine), fish, avian, reptile, cat, dog, ungulate, monkey, ape or human.

In cases where a candidate molecule is sufficiently basic or acidic to form stable nontoxic acid or base salts, administration of the candidate molecule as a salt may be appropriate. Examples of pharmaceutically acceptable salts are organic acid addition salts formed with acids that form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts. Pharmaceutically acceptable salts are obtained using standard procedures well known in the art, for example by reacting a sufficiently basic candidate molecule such as an amine with a suitable acid affording a physiologically acceptable anion. Alkali metal (e.g., sodium, potassium or lithium) or alkaline earth metal (e.g., calcium) salts of carboxylic acids also are made.

In some embodiments, a candidate molecule is administered systemically (e.g., orally) in combination with a pharmaceutically acceptable vehicle such as an inert diluent or an assimilable edible carrier. A candidate molecule may be enclosed in hard or soft shell gelatin capsules, compressed into tablets, or incorporated directly with the food of the patient's diet. For oral therapeutic administration, the active candidate molecule may be combined with one or more excipients and used in the form of ingestible tablets, buccal tablets, troches, capsules, elixirs, suspensions, syrups, wafers, and the like. Such compositions and preparations should contain at least 0.1% of active candidate molecule. The percentage of the compositions and preparations may be varied and may conveniently be between about 2 to about 60% of the weight of a given unit dosage form. The amount of active candidate molecule in such therapeutically useful compositions is such that an effective dosage level will be obtained.

Tablets, troches, pills, capsules, and the like also may contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active candidate molecule, sucrose or fructose as a sweetening agent, methyl and propylparabens as preservatives, a dye and flavoring such as cherry or orange flavor. Any material used in preparing any unit dosage form is pharmaceutically acceptable and substantially non-toxic in the amounts employed. In addition, the active candidate molecule may be incorporated into sustained-release preparations and devices.

The active candidate molecule also may be administered intravenously or intraperitoneally by infusion or injection. Solutions of the active candidate molecule or its salts may be prepared in a buffered solution, often phosphate buffered saline, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms. The candidate molecule is sometimes prepared as a polymatrix-containing formulation for such administration (e.g., a liposome or microsome). Liposomes are described for example in U.S. Pat. No. 5,703,055 (Felgner, et al.) and Gregoriadis, Liposome Technology vols. I to III (2nd ed. 1993).

Pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active candidate molecule in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present candidate molecules may be applied in liquid form. Candidate molecules often are administered as compositions or formulations, in combination with a dermatologically acceptable carrier, which may be a solid or a liquid. Examples of useful dermatological compositions used to deliver candidate molecules to the skin are known (see, e.g., Jacquet, et al. (U.S. Pat. No. 4,608,392), Geria (U.S. Pat. No. 4,992,478), Smith, et al. (U.S. Pat. No. 4,559,157) and Wortzman (U.S. Pat. No. 4,820,508).

Candidate molecules may be formulated with a solid carrier, which include finely divided solids such as talc, clay, microcrystalline cellulose, silica, alumina and the like. Useful liquid carriers include water, alcohols or glycols or water-alcohol/glycol blends, in which the present candidate molecules can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers. Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Nucleic acids having nucleotide sequences, or complements thereof, can be isolated and prepared in a composition for use and administration. A nucleic acid composition can include pharmaceutically acceptable salts, esters, or salts of such esters of one or more nucleic acids. Naked nucleic acids may be administered to a system, or nucleic acids may be formulated with one or more other molecules.

Compositions comprising nucleic acids can be prepared as a solution, emulsion, or polymatrix-containing formulation (e.g., liposome and microsphere). Examples of such compositions are set forth in U.S. Pat. Nos. 6,455,308 (Freier), 6,455,307 (McKay et al.), 6,451,602 (Popoff et al.), and 6,451,538 (Cowsert), and examples of liposomes also are described in U.S. Pat. No. 5,703,055 (Felgner et al.) and Gregoriadis, Lipsome Technology vols. I to III (2nd ed. 1993). The compositions can be prepared for any mode of administration, including topical, oral, pulmonary, parenteral, intrathecal, and intranutrical administration. Examples of compositions for particular modes of administration are set forth in U.S. Pat. Nos. 6,455,308 (Freier), 6,455,307 (McKay et al.), 6,451,602 (Popoff et al.), and 6,451,538 (Cowsert). Nucleic acid compositions may include one or more pharmaceutically acceptable carriers, excipients, penetration enhancers, and/or adjuncts. Choosing the combination of pharmaceutically acceptable salts, carriers, excipients, penetration enhancers, and/or adjuncts in the composition depends in part upon the mode of administration. Guidelines for choosing the combination of components for an nucleic acid oligonucleotide composition are known, and examples are set forth in U.S. Pat. Nos. 6,455,308 (Freier), 6,455,307 (McKay et al.), 6,451,602 (Popoff et al.), and 6,451,538 (Cowsert).

A nucleic acid may be modified by chemical linkages, moieties, or conjugates that reduce toxicity, enhance activity, cellular distribution, or cellular uptake of the nucleic acid. Examples of such modifications are set forth in U.S. Pat. Nos. 6,455,308 (Freier), 6,455,307 (McKay et al.), 6,451,602 (Popoff et al.), and 6,451,538 (Cowsert).

In another embodiment, a composition may comprise a plasmid that encodes a nucleic acid described herein. Many of the composition components described above for oligonucleotide compositions, such as carrier, excipient, penetration enhancer, and adjunct components, can be utilized in compositions containing expression plasmids. Also, the nucleic acid expressed by the plasmid may include some of the modifications described above that can be incorporated with or in an nucleic acid after expression by a plasmid. Recombinant plasmids are sometimes designed for nucleic acid expression in microbial cells (e.g., bacteria (e.g., E. coli.), yeast (e.g., S. cerviseae), or fungi), and more often the plasmids are designed for nucleic acid expression in eukaryotic cells (e.g., human cells). Suitable host cells are discussed further in Goeddel, Gene Expression Technology: Methods in Enzymology 185, Academic Press, San Diego, Calif. (1990). The plasmid may be delivered to the system or a portion of the plasmid that contains the nucleic acid encoding nucleotide sequence may be delivered.

When nucleic acids are expressed from plasmids in mammalian cells, expression plasmid regulatory elements sometimes are derived from viral regulatory elements. For example, commonly utilized promoters are derived from polyoma, Adenovirus 2, Rous Sarcoma virus, cytomegalovirus, and Simian Virus 40. A plasmid may include an inducible promoter operably linked to the nucleic acid-encoding nucleotide sequence. In addition, a plasmid sometimes is capable of directing nucleic acid expression in a particular cell type by use of a tissue-specific promoter operably linked to the nucleic acid-encoding sequence, examples of which are albumin promoters (liver-specific; Pinkert et al., Genes Dev. 1: 268-277 (1987)), lymphoid-specific promoters (Calame & Eaton, Adv. Immunol. 43: 235-275 (1988)), T-cell receptor promoters (Winoto & Baltimore, EMBO J. 8: 729-733 (1989)), immunoglobulin promoters (Banerji et al., Cell 33: 729-740 (1983) and Queen & Baltimore, Cell 33: 741-748 (1983)), neuron-specific promoters (e.g., the neurofilament promoter; Byrne & Ruddle, Proc. Natl. Acad. Sci. USA 86: 5473-5477 (1989)), pancreas-specific promoters (Edlund et al., Science 230: 912-916 (1985)), and mammary gland-specific promoters (e.g., milk whey promoter; U.S. Pat. No. 4,873,316 and European Application Publication No. 264,166). Developmentally-regulated promoters also may be utilized, which include, for example, murine hox promoters (Kessel & Gruss, Science 249: 374-379 (1990)) and α-fetopolypeptide promoters (Campes & Tilghman, Genes Dev. 3: 537-546 (1989)).

Nucleic acid compositions may be presented conveniently in unit dosage form, which are prepared according to conventional techniques known in the pharmaceutical industry. In general terms, such techniques include bringing the nucleic acid into association with pharmaceutical carrier(s) and/or excipient(s) in liquid form or finely divided solid form, or both, and then shaping the product if required. The nucleic acid compositions may be formulated into any dosage form, such as tablets, capsules, gel capsules, liquid syrups, soft gels, suppositories, and enemas. The compositions also may be formulated as suspensions in aqueous, non-aqueous, or mixed media. Aqueous suspensions may further contain substances which increase viscosity, including for example, sodium carboxymethylcellulose, sorbitol, and/or dextran. The suspension may also contain one or more stabilizers.

Nucleic acids can be translocated into cells via conventional transformation or transfection techniques. As used herein, the terms “transformation” and “transfection” refer to a variety of standard techniques for introducing an nucleic acid into a host cell, which include calcium phosphate or calcium chloride co-precipitation, transduction/infection, DEAE-dextran-mediated transfection, lipofection, electroporation, and iontophoresis. Also, liposome compositions described herein can be utilized to facilitate nucleic acid administration. An nucleic acid composition may be administered to an organism in a number of manners, including topical administration (including ophthalmic and mucous membrane (e.g., vaginal and rectal) delivery), pulmonary administration (e.g., inhalation or insufflation of powders or aerosols, including by nebulizer; intratracheal, intranasal, epidermal and transdermal), oral administration, and parenteral administration (e.g., intravenous, intraarterial, subcutaneous, intraperitoneal injection or infusion, intramuscular injection or infusion; and intracranial (e.g., intrathecal or intraventricular)).

Generally, the concentration of the candidate molecule or nucleic acid in a liquid composition often is from about 0.1 wt % to about 25 wt %, sometimes from about 0.5 wt % to about 10 wt %. The concentration in a semi-solid or solid composition such as a gel or a powder often is about 0.1 wt % to about 5 wt %, sometimes about 0.5 wt % to about 2.5 wt %. A candidate molecule or nucleic acid composition may be prepared as a unit dosage form, which is prepared according to conventional techniques known in the pharmaceutical industry. In general terms, such techniques include bringing a candidate molecule or nucleic acid into association with pharmaceutical carrier(s) and/or excipient(s) in liquid form or finely divided solid form, or both, and then shaping the product if required. The candidate molecule or nucleic acid composition may be formulated into any dosage form, such as tablets, capsules, gel capsules, liquid syrups, soft gels, suppositories, and enemas. The compositions also may be formulated as suspensions in aqueous, non-aqueous, or mixed media. Aqueous suspensions may further contain substances which increase viscosity, including for example, sodium carboxymethylcellulose, sorbitol, and/or dextran. The suspension may also contain one or more stabilizers.

The amount of the candidate molecule or nucleic acid, or an active salt or derivative thereof, required for use in treatment will vary not only with the particular salt selected but also with the route of administration, the nature of the condition being treated and the age and condition of the patient and will be ultimately at the discretion of the attendant physician or clinician. Candidate molecules or nucleic acids generally are used in amounts effective to achieve the intended purpose of reducing the number of targeted cells; detectably eradicating targeted cells; treating, ameliorating, alleviating, lessening, and removing symptoms of a disease or condition; and preventing or lessening the probability of the disease or condition or reoccurrence of the disease or condition. A therapeutically effective amount sometimes is determined in part by analyzing samples from a subject, cells maintained in vitro and experimental animals. For example, a dose can be formulated and tested in assays and experimental animals to determine an IC50 value for killing cells. Such information can be used to more accurately determine useful doses.

A useful candidate molecule or nucleic acid dosage often is determined by assessing its in vitro activity in a cell or tissue system and/or in vivo activity in an animal system. For example, methods for extrapolating an effective dosage in mice and other animals to humans are known to the art (see, e.g., U.S. Pat. No. 4,938,949). Such systems can be used for determining the LD50 (the dose lethal to 50% of the population) and the ED50 (the dose therapeutically effective in 50% of the population) of a candidate molecule or nucleic acid. The dose ratio between a toxic and therapeutic effect is the therapeutic index and it can be expressed as the ratio ED50/LD50. The candidate molecule or nucleic acid dosage often lies within a range of circulating concentrations for which the ED50 is associated with little or no toxicity. The dosage may vary within this range depending upon the dosage form employed and the route of administration utilized. For any candidate molecules or nucleic acids used in the methods described herein, the therapeutically effective dose can be estimated initially from cell culture assays. A dose sometimes is formulated to achieve a circulating plasma concentration range covering the IC50 (i.e., the concentration of the test candidate molecule which achieves a half-maximal inhibition of symptoms) as determined in in vitro assays, as such information often is used to more accurately determine useful doses in humans. Levels in plasma may be measured, for example, by high performance liquid chromatography.

Another example of effective dose determination for a subject is the ability to directly assay levels of “free” and “bound” candidate molecule or nucleic acid in the serum of the test subject. Such assays may utilize antibody mimics and/or “biosensors” generated by molecular imprinting techniques. The candidate molecule or nucleic acid is used as a template, or “imprinting molecule”, to spatially organize polymerizable monomers prior to their polymerization with catalytic reagents. Subsequent removal of the imprinted molecule leaves a polymer matrix which contains a repeated “negative image” of the candidate molecule and is able to selectively rebind the molecule under biological assay conditions (see, e.g., Ansell, et al., Current Opinion in Biotechnology 7: 89-94 (1996) and in Shea, Trends in Polymer Science 2: 166-173 (1994)). Such “imprinted” affinity matrixes are amenable to ligand-binding assays, whereby the immobilized monoclonal antibody component is replaced by an appropriately imprinted matrix (see, e.g., Vlatakis, et al., Nature 361: 645-647 (1993)). Through the use of isotope-labeling, “free” concentration of candidate molecule can be readily monitored and used in calculations of IC50. Such “imprinted” affinity matrixes can also be designed to include fluorescent groups whose photon-emitting properties measurably change upon local and selective binding of candidate molecule or nucleic acid. These changes can be readily assayed in real time using appropriate fiber optic devices, in turn allowing the dose in a test subject to be quickly optimized based on its individual IC₅₀. An example of such a “biosensor” is discussed in Kriz, et al., Analytical Chemistry 67: 2142-2144 (1995).

Exemplary doses include milligram or microgram amounts of the candidate molecule or nucleic acid per kilogram of subject or sample weight, for example, about 1 microgram per kilogram to about 500 milligrams per kilogram, about 100 micrograms per kilogram to about 5 milligrams per kilogram, or about 1 microgram per kilogram to about 50 micrograms per kilogram. It is understood that appropriate doses of a small molecule depend upon the potency of the small molecule with respect to the expression or activity to be modulated. When one or more of these small molecules is to be administered to an animal (e.g., a human) in order to modulate expression or activity of a polypeptide or nucleic acid described herein, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific candidate molecule employed, the age, body weight, general health, gender, and diet of the subject, the time of administration, the route of administration, the rate of excretion, any drug combination, and the degree of expression or activity to be modulated.

In some embodiments, a candidate molecule or nucleic acid is utilized to treat a cell proliferative condition. In such treatments, the terms “treating,” “treatment” and “therapeutic effect” can refer to reducing or stopping a cell proliferation rate (e.g., slowing or halting tumor growth), reducing the number of proliferating cancer cells (e.g., ablating part or all of a tumor) and alleviating, completely or in part, a cell proliferation condition. Cell proliferative conditions include, but are not limited to, cancers of the colorectum, breast, lung, liver, pancreas, lymph node, colon, prostate, brain, head and neck, skin, liver, kidney, and heart. Examples of cancers include hematopoietic neoplastic disorders, which are diseases involving hyperplastic/neoplastic cells of hematopoietic origin (e.g., arising from myeloid, lymphoid or erythroid lineages, or precursor cells thereof). The diseases can arise from poorly differentiated acute leukemias, e.g., erythroblastic leukemia and acute megakaryoblastic leukemia. Additional myeloid disorders include, but are not limited to, acute promyeloid leukemia (APML), acute myelogenous leukemia (AML) and chronic myelogenous leukemia (CML) (reviewed in Vaickus, Crit. Rev. in Oncol./Hemotol. 11:267-297 (1991)); lymphoid malignancies include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas include, but are not limited to non-Hodgkin lymphoma and variants thereof, peripheral T cell lymphomas, adult T cell leukemia/lymphoma (ATL), cutaneous T-cell lymphoma (CTCL), large granular lymphocytic leukemia (LGF), Hodgkin's disease and Reed-Steniberg disease. In a particular embodiment, the cell proliferative disorder is pancreatic cancer, including non-endocrine and endocrine tumors. Illustrative examples of non-endocrine tumors include but are not limited to adenocarcinomas, acinar cell carcinomas, adenosquamous carcinomas, giant cell tumors, intraductal papillary mucinous neoplasms, mucinous cystadenocarcinomas, pancreatoblastomas, serous cystadenomas, solid and pseudopapillary tumors. An endocrine tumor may be an islet cell tumor.

Kits

Kits comprise one or more containers, which contain one or more of the compositions and/or components described herein. A kit may comprise one or more of the components in any number of separate containers, packets, tubes, vials, microtiter plates and the like, and in some embodiments, the components may be combined in various combinations in such containers. A kit in some embodiments includes one reagent described herein and provides instructions that direct the user to another reagent described herein that is not included in the kit.

A kit sometimes is utilized in conjunction with a method described herein, and sometimes includes instructions for performing one or more methods described herein and/or a description of one or more compositions or reagents described herein. Instructions and/or descriptions may be in printed form and may be included in a kit insert. A kit also may include a written description of an internet location that provides such instructions or descriptions.

### EXAMPLES

The examples set forth below illustrate but do not limit the invention.

### Example 1

### Methods for Determining Quadruplex Formation and Conformation

Known assays can be utilized to determine whether a nucleic acid is capable of adopting a quadruplex structure. These assays include mobility shift assays, DMS methylation protection assays, polymerase arrest assays, transcription reporter assays, circular dichroism assays, and fluorescence assays.

### Gel Electrophoretic Mobility Shift Assay (EMSA)

An EMSA is useful for determining whether a nucleic acid forms a quadruplex and whether a nucleotide sequence is quadruplex-altering. EMSA is conducted as described previously (Jin & Pike, Mol. Endocrinol. 10: 196-205 (1996)) with minor modifications. Synthetic single-stranded oligonucleotides are labeled in the 5′-terminus with T4-kinase in the presence of [α-³²P] ATP (1,000 mCi/mmol, Amersham Life Science) and purified through a sephadex column. ³²P-labeled oligonucleotides (˜30,000 cpm) then are incubated with or without various concentrations of a testing compound in 20 μl of a buffer containing 10 mM Tris pH 7.5, 100 mM KCl, 5 mM dithiothreitol, 0.1 mM EDTA, 5 mM MgCl₂, 10% glycerol, 0.05% Nonedit P-40, and 0.1 mg/ml of poly(dI-dC) (Pharmacia). After incubation for 20 minutes at room temperature, binding reactions are loaded on a 5% polyacrylamide gel in 0.25× Tris borate-EDTA buffer (0.25×TBE, 1×TBE is 89 mM Tris-borate, pH 8.0, 1 mM EDTA). The gel is dried and each band is quantified using a phosphorimager.

DMS Methylation Protection Assay

Chemical footprinting assays are useful for assessing quadruplex structure. Quadruplex structure is assessed by determining which nucleotides in a nucleic acid are protected or unprotected from chemical modification as a result of being inaccessible or accessible, respectively, to the modifying reagent. A DMS methylation assay is an example of a chemical footprinting assay. In such an assay, bands from EMSA are isolated and subjected to DMS-induced strand cleavage. Each band of interest is excised from an electrophoretic mobility shift gel and soaked in 100 mM KCl solution (300 μl) for 6 hours at 4° C. The solutions are filtered (microcentrifuge) and 30,000 cpm (per reaction) of DNA solution is diluted further with 100 mM KCl in 0.1×TE to a total volume of 70 μl (per reaction). Following the addition of 1 μl salmon sperm DNA (0.1 μg/μl), the reaction mixture is incubated with 1 μl DMS solution (DMS:ethanol; 4:1; v:v) for a period of time. Each reaction is quenched with 18 μl of stop buffer (b-mercaptoathanol:water:NaOAc (3 M); 1:6:7; v:v:v). Following ethanol precipitation (twice) and piperidine cleavage, the reactions are separated on a preparative gel (16%) and visualized on a phosphorimager.

Polymerase Arrest Assay (Version 1)

An example of the Taq polymerase stop assay is described in Han et al., Nucl. Acids Res. 27: 537-542 (1999), which is a modification of that used by Weitzmalm et al., J. Biol. Chem. 271, 20958-20964 (1996). Briefly, a reaction mixture of template DNA (50 nM), Tris.HCl (50 mM), MgCl₂ (10 mM), DTT (0.5 mM), EDTA (0.1 mM), BSA (60 ng), and 5′-end-labeled quadruplex nucleic acid (˜18 nM) is heated to 90° C. for 5 minutes and allowed to cool to ambient temperature over 30 minutes. Taq Polymerase (1 μl) is added to the reaction mixture, and the reaction is maintained at a constant temperature for 30 minutes. Following the addition of 10 μl stop buffer (formamide (20 ml), 1 M NaOH (200 μl), 0.5 M EDTA (400 μl), and 10 mg bromophenol blue), the reactions are separated on a preparative gel (12%) and visualized on a phosphorimager. Adenine sequencing (indicated by “A” at the top of the gel) is performed using double-stranded DNA Cycle Sequencing System from Life Technologies. The general sequence for the template strands is TCCAACTATGTATAC-INSERT-TTAGCGACACGCAATTGCTATAGTGAGTCGTATTA. Bands on the gel that exhibit slower mobility are indicative of quadruplex formation.

Polymerase Arrest Assay (Version 2)

A 5′-fluorescent-labeled (FAM) primer (P45, 15 nM) is mixed with template DNA (15 nM) in a Tris-HCL buffer (15 mM Tris, pH 7.5) containing 10 mM MgCl₂, 0.1 mM EDTA and 0.1 mM mixed deoxynucleotide triphosphates (dNTP's). A FAM-P45 primer (5′-6FAM-AGTCTGAC TGACTGTACGTAGCTAATACGACTCACTATAGCAATT-3′) and the template DNA (5′-TCCAACTATGTATACTGGGGAGGGTGGGGAGGGTGGGGAAGGTTAGCGACACGCAATT GCTATAGTGAGTCGTATTAGCTACGTACAGTCAGTCAGACT-3′) is synthesized and HPLC purified (Applied Biosystems). The mixture is denatured at 95° C. for 5 minutes and, after cooling down to room temperature, is incubated at 37° C. for 15 minutes.

After cooling down to room temperature, 1 mM KCl₂ and the test compound (various concentrations) are added and the mixture incubated for 15 minutes at room temperature. The primer extension is performed by adding 10 mM KCl and Taq DNA Polymerase (2.5 U/reaction, Promega) and incubating at 70° C. for 30 minutes. The reaction is stopped by adding 1 μl of the reaction mixture to 10 μl Hi-Di Formamide mixed and 0.25 μl LIZ120 size standard. Hi-Di Formamide and LIZ120 size standard are utilized (Applied Biosystems). The products are separated and analyzed using capillary electrophoresis (ABI PRISM 3100-Avant Genetic Analyzer). The assay is performed using compounds described above and results are shown in Table 1. IC50 values can be calculated as the concentrations at which 50% of the DNA is arrested in the assay (i.e., the ratio of shorter partially extended DNA (arrested DNA) to full-length extended DNA is 1:1).

Transcription Reporter Assay

A luciferase promoter assay described in He et al., Science 281: 1509-1512 (1998) often is utilized for the study of quadruplex formation. Specifically, a vector utilized for the assay is set forth in reference 11 of the He et al document. In this assay, HeLa cells are transfected using the lipofectamin 2000-based system (Invitrogen) according to the manufacturer's protocol, using 0.1 μg of pRL-TK (Renilla luciferase reporter plasmid) and 0.9 μg of the quadruplex-forming plasmid. Firefly and Renilla luciferase activities are assayed using the Dual Luciferase Reporter Assay System (Promega) in a 96-well plate format according to the manufacturer's protocol.

Circular Dichroism Assay

Circular dichroism (CD) is utilized to determine whether another molecule interacts with a quadruplex nucleic acid. CD is particularly useful for determining whether a PNA or PNA-peptide conjugate hybridizes with a quadruplex nucleic acid in vitro. PNA probes are added to quadruplex DNA (5 μM each) in a buffer containing 10 mM potassium phosphate (pH 7.2) and 10 or 250 mM KCl at 37° C. and then allowed to stand for 5 min at the same temperature before recording spectra. CD spectra are recorded on a Jasco J-715 spectropolarimeter equipped with a thermoelectrically controlled single cell holder. CD intensity normally is detected between 220 nm and 320 nm and comparative spectra for quadruplex DNA alone, PNA alone, and quadruplex DNA with PNA are generated to determine the presence or absence of an interaction (see, e.g., Datta et al., JACS 123:9612-9619 (2001)). Spectra are arranged to represent the average of eight scans recorded at 100 nm/min.

Fluorescence Binding Assay

50 μl of quadruplex nucleic acid or a nucleic acid not capable of forming a quadruplex is added in 96-well plate. A test molecule or quadruplex-targeted nucleic acid also is added in varying concentrations. A typical assay is carried out in 100 μl of 20 mM HEPES buffer, pH 7.0, 140 mM NaCl, and 100 mM KCl. 50 μl of the signal molecule N-methylmesoporphyrin IX (NMM) then is added for a final concentration of 3 μM. NMM is obtained from Frontier Scientific Inc, Logan, Utah. Fluorescence is measured at an excitation wavelength of 420 μm and an emission wavelength of 660 nm using a FluoroStar 2000 fluorometer (BMG Labtechnologies, Durham, N.C.). Fluorescence often is plotted as a function of concentration of the test molecule or quadruplex-targeted nucleic acid and maximum fluorescent signals for NMM are assessed in the absence of these molecules.

### Example 2

### Identification of Conserved Nucleotide Sequences Conforming to Quadruplex Sequence Motifs

MotifFiles containing multiple alignments of the human genome (hg17, May 2004) to the following assemblies were searched for quadruplex motifs: Chimpanzee (November 2003 (panTro1)); Mouse (May 2004 (mm5)); Rat (June 2003 (m3)); Dog (July 2004 (canFam1)); Chicken (February 2004 (galGal2)); Fugu (August 2002 (fr1)); and Zebrafish (November 2003 (danRer1)). The chr*.maf.gz files each contained the alignments to the particular human chromosome. The aligned sequences came from the UCSC Genome Bioinformatics Site (http address hgdownload.cse.ucsc.edu/goldenPath/hg17/multiz8way/).

When a nucleotide sequence conforming to the quadruplex motif (a “quadruplex sequence”) was identified in the human genomic sequence, the aligned (animal) sequences were searched for any quadruplex sequences within the same area of the alignment (+−10 b.p. of the human quadruplex sequence). The human quadruplex sequence and the number of aligned sequences that also contained a quadruplex sequence in the same region were recorded. The human quadruplex sequence only was recorded if it was annotated as appearing in a gene or within 1000 b.p. upstream of a gene in the ENSEMBL annotation.

The quadruplex motif utilized for the searches were (G₃₊N₍₁₋₇₎)₃G₃₊ and (C₃₊N₍₁₋₇₎)₃C₃₊ motif, where G is guanine, C is cytosine, N is any nucleotide and “3+” is three or more nucleotides. The reverse complement of each human quadruplex sequence identified also was reported. The search identified:

13596 (G₃₊N₍₁₋₇₎)₃G₃₊ sequences found with at least two animal PQS also present;

13718 (C₃₊N₍₁₋₇₎)₃C₃₊ sequences found with at least two animal PQS also present;

3960 (G₃₊N₍₁₋₇₎)₃G₃₊ sequences found with at least three animal PQS also present;

4056 (C₃₊N₍₁₋₇₎)₃C₃+ sequences found with at least three animal PQS also present;

1572 (G₃₊N₍₁₋₇₎)₃G₃₊ sequences found with at least four animal PQS also present;

1587 (C₃₊N₍₁₋₇₎)₃C₃₊ sequences found with at least four animal PQS also present;

59 (G₃₊N₍₁₋₇₎)₃G₃₊ sequences found with at least five animal PQS also present; and

58 (C₃₊N₍₁₋₇₎)₃C₃₊ sequences found with at least five animal PQS also present.

Table B reports all human quadruplex sequences (and reverse complements for each) identified for all sequences alignments where four animal quadruplex sequences also were present (2038 sets of sequences). Table C reports all human quadruplex sequences (and reverse complements for each) identified for all sequence alignments where five animal quadruplex sequences also were present (84 sets of sequences).

TABLE B

| | |
|---|---|
| CCCTCCCTCCCTCCC | GGGAGGGAGGGAGGG |
| GGGTGGGAGGGTGGG | CCCACCCTCCCACCC |
| CCCACCCCCCCCACCC | GGGTGGGGGGGGTGGG |
| CCCCGCCCCCCCACCC | GGGTGGGGGGGCGGGG |
| CCCTCCCTCCCTTCCC | GGGAAGGGAGGGAGGG |
| CCCTGCCCACCCTCCC | GGGAGGGTGGGCAGGG |
| CCCTGCCCGCCCTCCC | GGGAGGGCGGGCAGGG |
| GGGAGGGGAGGGAGGG | CCCTCCCTCCCCTCCC |
| GGGAGGGTGGGCAGGG | CCCTGCCCACCCTCCC |
| GGGCCGGGAGGGAGGG | CCCTCCCTCCCGGCCC |
| GGGCGGGGAGGGAGGG | CCCTCCCTCCCCGCCC |
| GGGTGGGAGGGCTGGG | CCCAGCCCTCCCACCC |
| CCCACCCCCGCCCCCCC | GGGGGGGCGGGGGTGGG |
| CCCATCCCGCCCAGCCC | GGGCTGGGCGGGATGGG |
| CCCCTGCCCACCCACCC | GGGTGGGTGGGCAGGGG |
| CCCGCCCCCCCCCACCC | GGGTGGGGGGGGGCGGG |
| CCCGCCCCCGCCCGCCC | GGGCGGGCGGGGGCGGG |
| CCCGCCCGCCCCCGCCC | GGGCGGGGGCGGGCGGG |
| CCCTCCCCACCCGACCC | GGGTCGGGTGGGGAGGG |
| CCCTCCCTCCCTCCCCC | GGGGGAGGGAGGGAGGG |
| CCCTCCCTTCCCCTCCC | GGGAGGGGAAGGGAGGG |
| CCCTGCCCACCCTGCCC | GGGCAGGGTGGGCAGGG |
| CCCTTCCCACCCGCCCC | GGGGCGGGTGGGAAGGG |
| GGGAGGGGCGGGAAGGG | CCCTTCCCGCCCCTCCC |
| GGGAGGGGCGGGGCGGG | CCCGCCCCGCCCCTCCC |
| GGGAGTGGGCGGGCGGG | CCCGCCCGCCCACTCCC |
| GGGCAGGGTGGGCAGGG | CCCTGCCCACCCTGCCC |
| GGGCATGGGAGGGAGGG | CCCTCCCTCCCATGCCC |
| GGGCCGGGCGGGTGGGG | CCCCACCCGCCCGGCCC |
| GGGCGGGCGCGGGAGGG | CCCTCCCGCGCCCGCCC |
| GGGCGGGGCAGGGCGGG | CCCGCCCTGCCCCGCCC |
| GGGCGGGGGTGGGAGGG | CCCTCCCACCCCCGCCC |
| GGGGAGGGCGGGTCGGG | CCCGACCCGCCCTCCCC |
| GGGGCAGGGAGGGAGGG | CCCTCCCTCCCTGCCCC |
| GGGGCGGGCGGGGCGGG | CCCGCCCCGCCCGCCCC |
| GGGGGGGAGGGGGAGGG | CCCTCCCCCTCCCCCCC |
| GGGTGGGTGGGGCAGGG | CCCTGCCCCACCCACCC |
| CCCAGACCCGCCCCTCCC | GGGAGGGGCGGGTCTGGG |
| CCCAGCCCTGCCCAGCCC | GGGCTGGGCAGGGCTGGG |
| CCCATCCCAACCCCTCCC | GGGAGGGGTTGGGATGGG |
| CCCCCCCTTCCCCAACCC | GGGTTGGGGAAGGGGGGG |
| CCCCGACCCCTCCCACCC | GGGTGGGAGGGGTCGGGG |
| CCCCGCCCACTCCCGCCC | GGGCGGGAGTGGGCGGGG |
| CCCCTCCCCTCCCACCCC | GGGGTGGGAGGGGAGGGG |
| CCCGCCCCCACCCAGCCC | GGGCTGGGTGGGGGCGGG |
| CCCGCCCCCTCCCTCCCC | GGGGAGGGAGGGGGCGGG |
| CCCGTCCCTCCCACCCCC | GGGGGTGGGAGGGACGGG |
| CCCTCCCACCCCAGCCCC | GGGGCTGGGGTGGGAGGG |
| CCCTCCCACTACCCACCC | GGGTGGGTAGTGGGAGGG |
| CCCTCCCTCCCCAGACCC | GGGTCTGGGGAGGGAGGG |
| CCCTCCCTGCCCCCACCC | GGGTGGGGGCAGGGAGGG |
| CCCTCTCCCACCCCTCCC | GGGAGGGGTGGGAGAGGG |
| CCCTGCCCTGCCCTGCCC | GGGCAGGGCAGGGCAGGG |
| CCCTTTCCCTGCCCACCC | GGGTGGGCAGGGAAAGGG |
| GGGAGCGGGCGGGGCGGG | CCCGCCCCGCCCGCTCCC |
| GGGAGGGCAGGGCCAGGG | CCCTGGCCCTGCCCTCCC |
| GGGAGGGCAGGGTCAGGG | CCCTGACCCTGCCCTCCC |
| GGGAGGGGGCGGGTGGGG | CCCCACCCGCCCCCTCCC |
| GGGAGGGTGGGAGTGGGG | CCCCACTCCCACCCTCCC |
| GGGCGGGGAGGGGCGGGG | CCCCGCCCCTCCCCGCCC |
| GGGCGGGGTAGGGCGGGG | CCCCGCCCTACCCCGCCC |
| GGGCGGGTGGGGGCGGGG | CCCCGCCCCCACCCGCCC |
| GGGCTGGGCTGGGCTGGG | CCCAGCCCAGCCCAGCCC |
| GGGCTGGGTCGGGGAGGG | CCCTCCCCGACCCAGCCC |
| GGGGAGGGTGGGGAAGGG | CCCTTCCCCACCCTCCCC |
| GGGGGCGGGAAGGGTGGG | CCCACCCTTCCCGCCCCC |
| GGGGGCGGGGCGGGTGGG | CCCACCCGCCCCGCCCCC |
| GGGGGTGGGGTGGGAGGG | CCCTCCCACCCCACCCCC |
| GGGGTGGGGGAGGGGGGG | CCCCCCCTCCCCCACCCC |
| GGGGTTGGGGTGGGAGGG | CCCTCCCACCCCAACCCC |
| GGGTCGGGGCAGGGAGGG | CCCTCCCTGCCCCGACCC |
| GGGTGGGGTGGGACTGGG | CCCAGTCCCACCCCACCC |
| GGGTGGGTGGGTTGTGGG | CCCACAACCCACCCACCC |
| CCCACCCCACCCAACCCCC | GGGGGTTGGGTGGGGTGGG |
| CCCACCCCCCTGCCCTCCC | GGGAGGGCAGGGGGGTGGG |
| CCCACCCTAGCCCTGGCCC | GGGCCAGGGCTAGGGTGGG |
| CCCACCCTTGCCCAATCCC | GGGATTGGGCAAGGGTGGG |
| CCCAGCCCTCCCGTTGCCC | GGGCAACGGGAGGGCTGGG |
| CCCATTCCCAGCCCAGCCC | GGGCTGGGCTGGGAATGGG |
| CCCCACCCGGACCCCACCC | GGGTGGGGTCCGGGTGGGG |
| CCCCCCCGCCCGCCCGCCC | GGGCGGGCGGGCGGGGGGG |
| CCCCCTCCCTTCCCAGCCC | GGGCTGGGAAGGGAGGGGG |
| CCCCGACCCACCCCATCCC | GGGATGGGGTGGGTCGGGG |
| CCCCGACCCAGGCCCTCCC | GGGAGGGCCTGGGTCGGGG |
| CCCCGCCCCGCCCCGCCCC | GGGGCGGGGCGGGGCGGGG |
| CCCCGCCCCTCCCCTGCCC | GGGCAGGGGAGGGGCGGGG |
| CCCCGCCCTCCCCCGACCC | GGGTCGGGGGAGGGCGGGG |
| CCCCTCCCCCCCGCGCCCC | GGGGCGCGGGGGGGAGGGG |
| CCCCTCCCCCGCCCGTCCC | GGGACGGGCGGGGGAGGGG |
| CCCCTTCCCCTCCCCTCCC | GGGAGGGGAGGGGAAGGGG |
| CCCGAGCTCCCTCCCGCCC | GGGCGGGAGGGAGCTCGGG |
| CCCGCCCCTCCCTCGCCCC | GGGGCGAGGGAGGGGCGGG |
| CCCGCCCGCCCGCCGGCCC | GGGCCGGCGGGCGGGCGGG |
| CCCGCCCGCCCTCCCTCCC | GGGAGGGAGGGCGGGCGGG |
| CCCGCCCTCCCGGAGGCCC | GGGCCTCCGGGAGGGCGGG |
| CCCGCTGCCCGCCCCTCCC | GGGAGGGGCGGGCAGCGGG |
| CCCTCCCACCCCCTTTCCC | GGGAAAGGGGGTGGGAGGG |
| CCCTCCCCTACCCCCTCCC | GGGAGGGGGTAGGGGAGGG |
| CCCTCCCTCTGCCCCCCCC | GGGGGGGGCAGAGGGAGGG |
| CCCTCCCTCTTTCCCACCC | GGGTGGGAAAGAGGGAGGG |
| CCCTCCCTTCCCCCACCCC | GGGGTGGGGGAAGGGAGGG |
| CCCTGCCCATGCCCACCCC | GGGGTGGGCATGGGCAGGG |
| CCCTTCCCCCAGCCCTCCC | GGGAGGGCTGGGGGAAGGG |
| CCCTTCCCTTCCCTGCCCC | GGGGCAGGGAAGGGAAGGG |
| GGGAAGGGACTGGGGAGGG | CCCTCCCCAGTCCCTTCCC |
| GGGAAGGGGACAGGGAGGG | CCCTCCCTGTCCCCTTCCC |
| GGGAGAGGGAGAGGGCGGG | CCCGCCCTCTCCCTCTCCC |
| GGGAGGGGCGGGAGGGGGG | CCCCCCTCCCGCCCCTCCC |
| GGGAGGGGGCAGGGCTGGG | CCCAGCCCTGCCCCCTCCC |
| GGGAGTGGGAGGGATGGGG | CCCCATCCCTCCCACTCCC |
| GGGATGGGGGAGGGTGGGG | CCCCACCCTCCCCCATCCC |
| GGGCATGGGAAGGGTGGGG | CCCCACCCTTCCCATGCCC |
| GGGCCGGGTGGGGGTAGGG | CCCTACCCCCACCCGGCCC |
| GGGCGGGAGGGCTGGCGGG | CCCGCCAGCCCTCCCGCCC |
| GGGCGGGAGGGGGGAGGGG | CCCCTCCCCCCTCCCGCCC |
| GGGCGGGCGGGCCAGCGGG | CCCGCTGGCCCGCCCGCCC |
| GGGCGGGCTATGGGCGGGG | CCCCGCCCATAGCCCGCCC |
| GGGCGGGGATGGGGAGGGG | CCCCTCCCCATCCCCGCCC |
| GGGCGGGGCCTGGGCGGGG | CCCCGCCCAGGCCCCGCCC |
| GGGCGGGGTGGGCCCAGGG | CCCTGGGCCCACCCCGCCC |
| GGGCGGGTCCTGGGCCGGG | CCCGGCCCAGGACCCGCCC |
| GGGCGGGTCTGTGGGTGGG | CCCACCCACAGACCCGCCC |
| GGGCTGGGCTGTGGGTGGG | CCCACCCACAGCCCAGCCC |
| GGGCTTGGGGGGGTGGGGG | CCCCCACCCCCCCAAGCCC |
| GGGGAAAGGGGAGGGAGGG | CCCTCCCTCCCCTTTCCCC |
| GGGGAAATGGGTGGGAGGG | CCCTCCCACCCATTTCCCC |
| GGGGAAGGGACGGGGTGGG | CCCACCCCGTCCCTTCCCC |
| GGGGAGGGGCGGGGCGGGG | CCCCGCCCCGCCCCTCCCC |
| GGGGAGGGTTTGGGGTGGG | CCCACCCCAAACCCTCCCC |
| GGGGCGGGGGTGGGGAGGG | CCCTCCCCACCCCCGCCCC |
| GGGGCTGGGGAGGGCGGGG | CCCCGCCCTCCCCAGCCCC |
| GGGGGAGGGGAAGGGTGGG | CCCACCCTTCCCCTCCCCC |
| GGGGGCGGGAGTGGGGGGG | CCCCCCCACTCCCGCCCCC |
| GGGGGCGGGGCGGGGGGGG | CCCCCCCCGCCCCGCCCCC |
| GGGGGGGCCATGGGGCGGG | CCCGCCCCATGGCCCCCCC |
| GGGGGTGGGAGGGAGGGGG | CCCCCTCCCTCCCACCCCC |
| GGGGTGGGAGAGGGCTGGG | CCCAGCCCTCTCCCACCCC |
| GGGGTGGGCGCGGGGCGGG | CCCGCCCCGCGCCCACCCC |
| GGGGTGGGGAGGGTGGGGG | CCCCCACCCTCCCCACCCC |
| GGGGTGGGGCGGGAGGGGG | CCCCCTCCCGCCCCACCCC |
| GGGGTGGGGTGGGAGTGGG | CCCACTCCCACCCCACCCC |
| GGGTAAGGGAGGGAAGGGG | CCCCTTCCCTCCCTTACCC |
| GGGTAGGGAGTGGGCTGGG | CCCAGCCCACTCCCTACCC |
| GGGTCCCGGGATGGGGGGG | CCCCCCCATCCCGGGACCC |
| GGGTCGGGGAGAGGGAGGG | CCCTCCCTCTCCCCGACCC |
| GGGTGGGAATGGGGTAGGG | CCCTACCCCATTCCCACCC |
| GGGTGGGGAGGGGGGGGGG | CCCGCCCCCCTCCCCACCC |
| GGGTGGGGCTGGGGTGGGG | CCCCACCCCAGCCCCACCC |
| GGGTGGGGGAAGGGCGGGG | CCCCGCCCTTCCCCCACCC |
| GGGTGGGGGGAGGGCTGGG | CCCAGCCCTCCCCCCACCC |
| GGGTGGGGGGGAGGGGGGG | CCCCCCCTCCCCCCCACCC |
| CCCACCCCGCCCACCCACCC | GGGTGGGTGGGCGGGGTGGG |
| CCCACCCCGGTCCCCACCCC | GGGGTGGGGACCGGGGTGGG |
| CCCACCCTCACCCCACCCCC | GGGGGTGGGGTGAGGGTGGG |
| CCCACTTTTCCCACCCACCC | GGGTGGGTGGGAAAAGTGGG |
| CCCAGCCCCAGCCCCAGCCC | GGGCTGGGGCTGGGGCTGGG |
| CCCAGCCCCCAACCCCACCC | GGGTGGGGTTGGGGGCTGGG |
| CCCAGCCCTGCCCCTCGCCC | GGGCGAGGGGCAGGGCTGGG |
| CCCAGGCCCCCCCGGCCCCC | GGGGGCCGGGGGGGCCTGGG |
| CCCAGGTGCCCGCCCCACCC | GGGTGGGGCGGGCACCTGGG |
| CCCATCCCATCCCCCTTCCC | GGGAAGGGGGATGGGATGGG |
| CCCATCCCCATCCCCATCCC | GGGATGGGGATGGGGATGGG |
| CCCCACCCGCACCCCACCCC | GGGGTGGGGTGCGGGTGGGG |
| CCCCAGCCCACCCCATCCCC | GGGGATGGGGTGGGCTGGGG |
| CCCCCAGCCCCGCCCGTCCC | GGGACGGGCGGGGCTGGGGG |
| CCCCCATCCCTGTCCCACCC | GGGTGGGACAGGGATGGGGG |
| CCCCCCCCACCCTCATCCCC | GGGGATGAGGGTGGGGGGGG |
| CCCCCTCCCCAGACCCTCCC | GGGAGGGTCTGGGGAGGGGG |
| CCCCCTCCCCCTACCCCCCC | GGGGGGGTAGGGGGAGGGGG |
| CCCCGCCCCACGGCCCCCCC | GGGGGGGCCGTGGGGCGGGG |
| CCCCGCCCCCGCTCCCACCC | GGGTGGGAGCGGGGGCGGGG |
| CCCCGCCCCTCCCACACCCC | GGGGTGTGGGAGGGGCGGGG |
| CCCCGCCCCTCCCCCTTCCC | GGGAAGGGGGAGGGGCGGGG |
| CCCCGCCCTCTGCCCGCCCC | GGGGCGGGCAGAGGGCGGGG |
| CCCCGGCCCCGCCCACCCCC | GGGGGTGGGCGGGGCCGGGG |
| CCCCTCCCGGGCCCTGGCCC | GGGCCAGGGCCCGGGAGGGG |
| CCCCTCCCTCAGCCCTCCCC | GGGGAGGGCTGAGGGAGGGG |
| CCCCTGCCCGCCCATGGCCC | GGGCCATGGGCGGGCAGGGG |
| CCCCTGCCCTCCCCCCTCCC | GGGAGGGGGGAGGGCAGGGG |
| CCCCTTCCCGCCCTCCCCCC | GGGGGGAGGGCGGGAAGGGG |
| CCCGAACCCCAAGCCCTCCC | GGGAGGGCTTGGGGTTCGGG |
| CCCGCCCCCTAAACCCCCCC | GGGGGGGTTTAGGGGGCGGG |
| CCCGCCCGCGCCCCGCCCCC | GGGGGCGGGGCGCGGGCGGG |
| CCCGCCCTTCGCCCAGCCCC | GGGGCTGGGCGAAGGGCGGG |
| CCCGGCCCGCCGCCCAGCCC | GGGCTGGGCGGCGGGCCGGG |
| CCCGGGCCCAGGCCCAACCC | GGGTTGGGCCTGGGCCCGGG |
| CCCTCACCCACCCCCAACCC | GGGTTGGGGGTGGGTGAGGG |
| CCCTCCCAAGCCCGCGCCCC | GGGGCGCGGGCTTGGGAGGG |
| CCCTCCCAGCCCCCACCCCC | GGGGGTGGGGGCTGGGAGGG |
| CCCTCCCAGTGACCCCACCC | GGGTGGGGTCACTGGGAGGG |
| CCCTCCCATCCCCCACCCCC | GGGGGTGGGGGATGGGAGGG |
| CCCTCCCCACTCCCCACCCC | GGGGTGGGGAGTGGGGAGGG |
| CCCTCCCCCACCCCACCCCC | GGGGGTGGGGTGGGGGAGGG |
| CCCTCCCCTATCCCCGCCCC | GGGGCGGGGATAGGGGAGGG |
| CCCTCCCCTCCCCCGGACCC | GGGTCCGGGGGAGGGGAGGG |
| CCCTCCCTCCCCACCCGCCC | GGGCGGGTGGGGAGGGAGGG |
| CCCTCCCTTCCCTGTTTCCC | GGGAAACAGGGAAGGGAGGG |
| CCCTCTTCCCGCCCCGCCCC | GGGGCGGGGCGGGAAGAGGG |
| CCCTTCCCCGGCCCCCTCCC | GGGAGGGGGCCGGGGAAGGG |
| GGGAAGGGAGGGGCTGGGGG | CCCCCAGCCCCTCCCTTCCC |
| GGGACTGGGGAGGGCGAGGG | CCCTCGCCCTCCCCAGTCCC |
| GGGAGAGGGGCATGGGAGGG | CCCTCCCATGCCCCTCTCCC |
| GGGAGGGAAGGAGGGGAGGG | CCCTCCCCTCCTTCCCTCCC |
| GGGAGGGAGGGAGGGGAGGG | CCCTCCCCTCCCTCCCTCCC |
| GGGAGGGGGAGGGACGAGGG | CCCTCGTCCCTCCCCCTCCC |
| GGGAGGGGGCTGGGGGTGGG | CCCACCCCCAGCCCCCTCCC |
| GGGAGGGGGTGGGGGGAGGG | CCCTCCCCCCACCCCCTCCC |
| GGGAGTGGGGTGGGGGAGGG | CCCTCCCCCACCCCACTCCC |
| GGGATGGGGTGGGGGAGGGG | CCCCTCCCCCACCCCATCCC |
| GGGCAGGGGGAGGGGGAGGG | CCCTCCCCCTCCCCCTGCCC |
| GGGCCGGGTAGGGGGGCGGG | CCCGCCCCCCTACCCGGCCC |
| GGGCGGGAGGAGTGGGTGGG | CCCACCCACTCCTCCCGCCC |
| GGGCGGGCACCTGGGGTGGG | CCCACCCCAGGTGCCCGCCC |
| GGGCGGGGCCCCCGGGCGGG | CCCGCCCGGGGGCCCCGCCC |
| GGGCTGGGCAGGGCTGAGGG | CCCTCAGCCCTGCCCAGCCC |
| GGGCTGGGGCAGGGGTGGGG | CCCCACCCCTGCCCCAGCCC |
| GGGGAAGGGAGGGGGGCGGG | CCCGCCCCCCTCCCTTCCCC |
| GGGGACGGGGCGGGGGAGGG | CCCTCCCCCGCCCCGTCCCC |
| GGGGCGGGCCGCGGGTGGGG | CCCCACCCGCGGCCCGCCCC |
| GGGGCGGGGCCGGGGGCGGG | CCCGCCCCCGGCCCCGCCCC |
| GGGGGAGGGCTCCGGGAGGG | CCCTCCCGGAGCCCTCCCCC |
| GGGGGAGGGGTGGGAATGGG | CCCATTCCCACCCCTCCCCC |
| GGGGGCCCAGGGCGGGAGGG | CCCTCCCGCCCTGGGCCCCC |
| GGGGGCGGGGCGGGGGAGGG | CCCTCCCCCGCCCCGCCCCC |
| GGGGGGGCCATGGGGGCGGG | CCCGCCCCCATGGCCCCCCC |
| GGGGGTGGGAAGGGGGCGGG | CCCGCCCCCTTCCCACCCCC |
| GGGGGTGGGAGAGGGAGGGG | CCCCTCCCTCTCCCACCCCC |
| GGGGTGAGGGAAGGGAAGGG | CCCTTCCCTTCCCTCACCCC |
| GGGGTGGGGTGGGGAGAGGG | CCCTCTCCCCACCCCACCCC |
| GGGGTGGGTGCGGGGCAGGG | CCCTGCCCCGCACCCACCCC |
| GGGTAGGGGGGAGGGTAGGG | CCCTACCCTCCCCCCTACCC |
| GGGTCGGGGGCGGGGCTGGG | CCCAGCCCCGCCCCCGACCC |
| GGGTGCAGGGAGGGGTGGGG | CCCCACCCCTCCCTGCACCC |
| GGGTGGGAGCCTGGGCTGGG | CCCAGCCCAGGCTCCCACCC |
| GGGTGGGCTACGGGAGAGGG | CCCTCTCCCGTAGCCCACCC |
| GGGTGGGGAGGGAGGTGGGG | CCCCACCTCCCTCCCCACCC |
| GGGTTGGGGGCGGGGGTGGG | CCCACCCCCGCCCCCAACCC |
| CCCACCAGCCCACCCACCCCC | GGGGGTGGGTGGGCTGGTGGG |
| CCCACCCACCCAACTGGTCCC | GGGACCAGTTGGGTGGGTGGG |
| CCCACCCCGCCCACCACCCCC | GGGGGTGGTGGGCGGGGTGGG |
| CCCACCCCTCCCTCCTCCCCC | GGGGGAGGAGGGAGGGGTGGG |
| CCCACCCTCCCCAGACAGCCC | GGGCTGTCTGGGGAGGGTGGG |
| CCCACCCTCCTGCCCATCCCC | GGGGATGGGCAGGAGGGTGGG |
| CCCACTCCCCCTCCCCTCCCC | GGGGAGGGGAGGGGGAGTGGG |
| CCCATCCCAGTCCCTGTCCCC | GGGGACAGGGACTGGGATGGG |
| CCCATCCCCCGCCCCAGCCCC | GGGGCTGGGGCGGGGGATGGG |
| CCCATTCCCACCCACCCACCC | GGGTGGGTGGGTGGGAATGGG |
| CCCCAACCCCAGCCCACCCCC | GGGGGTGGGCTGGGGTTGGGG |
| CCCCACACCCACCCCAGACCC | GGGTCTGGGGTGGGTGTGGGG |
| CCCCACCCCACCTCCCCACCC | GGGTGGGGAGGTGGGGTGGGG |
| CCCCACCCCCTCCCACCACCC | GGGTGGTGGGAGGGGGTGGGG |
| CCCCACCCCCTCTCCCTTCCC | GGGAAGGGAGAGGGGGTGGGG |
| CCCCCAACCCCTGCCCTCCCC | GGGGAGGGCAGGGGTTGGGGG |
| CCCCCACCCGCTCCCCGCCCC | GGGGCGGGGAGCGGGTGGGGG |
| CCCCCAGCCCACCCAGGCCCC | GGGGCCTGGGTGGGCTGGGGG |
| CCCCCCACCCACCCTGAACCC | GGGTTCAGGGTGGGTGGGGGG |
| CCCCCCCTCCCCCGCCCGCCC | GGGCGGGCGGGGGAGGGGGGG |
| CCCCCGCCCCGGCTCCCACCC | GGGTGGGAGCCGGGGCGGGGG |
| CCCCCGGCCCTCCCTGCGCCC | GGGCGCAGGGAGGGCCGGGGG |
| CCCCCTCCCCATCCCACCCCC | GGGGGTGGGATGGGGAGGGGG |
| CCCCGCCCCCGCCCGCCGCCC | GGGCGGCGGGCGGGGGCGGGG |
| CCCCGCCCGGCCCGTACACCC | GGGTGTACGGGCCGGGCGGGG |
| CCCCGCCCTCACCCTTCTCCC | GGGAGAAGGGTGAGGGCGGGG |
| CCCCTACCCTGCCCCCGCCCC | GGGGCGGGGGCAGGGTAGGGG |
| CCCCTAGCCCAAACCCTACCC | GGGTAGGGTTTGGGCTAGGGG |
| CCCCTCCCCAGCCCCCTTCCC | GGGAAGGGGGCTGGGGAGGGG |
| CCCCTCCCCCCTCCCCAGCCC | GGGCTGGGGAGGGGGGAGGGG |
| CCCCTCCCCCTGCCCTTTCCC | GGGAAAGGGCAGGGGGAGGGG |
| CCCCTCCCGCGCCCTCCACCC | GGGTGGAGGGCGCGGGAGGGG |
| CCCCTCCCTCCGCCCACGCCC | GGGCGTGGGCGGAGGGAGGGG |
| CCCCTCCCTCTCTCCCGCCCC | GGGGCGGGAGAGAGGGAGGGG |
| CCCCTCGCCCCGCCCCTTCCC | GGGAAGGGGCGGGGCGAGGGG |
| CCCCTGCCCCTCCCATTTCCC | GGGAAATGGGAGGGGCAGGGG |
| CCCGAACCCCCCCTCCCCCCC | GGGGGGGAGGGGGGGTTCGGG |
| CCCGACCCCCTCCCCACGCCC | GGGCGTGGGGAGGGGGTCGGG |
| CCCGCCCCCGCCCCCTCGCCC | GGGCGAGGGGGCGGGGGCGGG |
| CCCGCTCCCGCCCCCGCTCCC | GGGAGCGGGGGCGGGAGCGGG |
| CCCGGCCCCAAACCCTGGCCC | GGGCCAGGGTTTGGGGCCGGG |
| CCCGGCCCCCCCCAACCCCCC | GGGGGGTTGGGGGGGGCCGGG |
| CCCGGCCCCGCCTCCCGCCCC | GGGGCGGGAGGCGGGGCCGGG |
| CCCGGCCCCGGCCCCCGCCCC | GGGGCGGGGGCCGGGGCCGGG |
| CCCGGCCGCCCCTCCCGCCCC | GGGGCGGGAGGGGCGGCCGGG |
| CCCGGGCCCAGCCCTGTGCCC | GGGCACAGGGCTGGGCCCGGG |
| CCCTAACCCTCCACCCTCCCC | GGGGAGGGTGGAGGGTTAGGG |
| CCCTCCCACTCCGCCCCACCC | GGGTGGGGCGGAGTGGGAGGG |
| CCCTCCCCGCACCCGCGTCCC | GGGACGCGGGTGCGGGGAGGG |
| CCCTCCCTCCCTTTCCCTCCC | GGGAGGGAAAGGGAGGGAGGG |
| CCCTCCCTCCTTGCCCCCCCC | GGGGGGGGCAAGGAGGGAGGG |
| CCCTCTCCCCTCCCCCCACCC | GGGTGGGGGGAGGGGAGAGGG |
| CCCTGCCCAGCTGCCCGCCCC | GGGGCGGGCAGCTGGGCAGGG |
| CCCTGGCCCCGGCCCCGGCCC | GGGCCGGGGCCGGGGCCAGGG |
| CCCTTTCCCAGGCCCCCACCC | GGGTGGGGGCCTGGGAAAGGG |
| GGGAAGAGGGGGGTGGGTGGG | CCCACCCACCCCCCTCTTCCC |
| GGGAAGGAGGGGCGGGGTGGG | CCCACCCCGCCCCTCCTTCCC |
| GGGAATGTGGGCTGGGGAGGG | CCCTCCCCAGCCCACATTCCC |
| GGGAGAGAGAGGGAGGGAGGG | CCCTCCCTCCCTCTCTCTCCC |
| GGGAGGCGGGAGGAGGGAGGG | CCCTCCCTCCTCCCGCCTCCC |
| GGGAGGGAGGAGGGAAGAGGG | CCCTCTTCCCTCCTCCCTCCC |
| GGGAGGGAGGGAGAGAAAGGG | CCCTTTCTCTCCCTCCCTCCC |
| GGGAGGGGACACGGGGGCGGG | CCCGCCCCCGTGTCCCCTCCC |
| GGGAGGGGCCGGGGGCCGGGG | CCCCGGCCCCCGGCCCCTCCC |
| GGGAGGGGGTGGGAGGGGGGG | CCCCCCCTCCCACCCCCTCCC |
| GGGAGGGGGTGGGGGAGCGGG | CCCGCTCCCCCACCCCCTCCC |
| GGGCAGGGGATGAGGGGAGGG | CCCTCCCCTCATCCCCTGCCC |
| GGGCATGGGCATGGGCATGGG | CCCATGCCCATGCCCATGCCC |
| GGGCCAGGGCAGGGGCTGGGG | CCCCAGCCCCTGCCCTGGCCC |
| GGGCCGGGGCGGGGGCGGGGG | CCCCCGCCCCCGCCCCCGGCC |
| GGGCGGAGGGCGGGGCGTGGG | CCCACGCCCCGCCCTCCGCCC |
| GGGCGGGCGGGGGCGGGGGGG | CCCCCCCGCCCCCGCCCGCCC |
| GGGCGGGGAAGGGGGGTTGGG | CCCAACCCCCCTTCCCCGCCC |
| GGGCGGGGAGGCGGGGCAGGG | CCCTGCCCCGCCTCCCCGCCC |
| GGGCTCCCTGGGTGGGATGGG | CCCATCCCACCCAGGGAGCCC |
| GGGCTGGCGGGTGGGGAGGGG | CCCCTCCCCACCCGCCAGCCC |
| GGGCTGGGACCCGGGCTGGGG | CCCCAGCCCGGGTCCCAGCCC |
| GGGCTGGGCAGGGGGAAGGGG | CCCCTTCCCCCTGCCCAGCCC |
| GGGCTGGGCCTCTGGGGAGGG | CCCTCCCCAGAGGCCCAGCCC |
| GGGCTGGGGCGGGCCGGCGGG | CCCGCCGGCCCGCCCCAGCCC |
| GGGGAAGGGTGGGGTTGGGGG | CCCCCAACCCCACCCTTCCCC |
| GGGGAATGGGGTGGGGGTGGG | CCCACCCCCACCCCATTCCCC |
| GGGGACAGGGCGGGCTAGGGG | CCCCTAGCCCGCCCTGTCCCC |
| GGGGAGAGGGCGGGAGGCGGG | CCCGCCTCCCGCCCTCTCCCC |
| GGGGAGGGGACGGGAAAGGGG | CCCCTTTCCCGTCCCCTCCCC |
| GGGGAGGGGTGGGGGGAGGGG | CCCCTCCCCCCACCCCTCCCC |
| GGGGATGGGAGGGTGCCGGGG | CCCCGGCACCCTCCCATCCCC |
| GGGGCAGGGAGCCGGGGTGGG | CCCACCCCGGCTCCCTGCCCC |
| GGGGCCGGGGAGGGAGGCGGG | CCCGCCTCCCTCCCCGGCCCC |
| GGGGCGGGGAGGGGCTGCGGG | CCCGCAGCCCCTCCCCGCCCC |
| GGGGCGGGGGTGGGGCCGGGG | CCCCGGCCCCACCCCCGCCCC |
| GGGGCGTGGGGGCGGGGAGGG | CCCTCCCCGCCCCCACGCCCC |
| GGGGCTGGGGTGGAGGGCGGG | CCCGCCCTCCACCCCAGCCCC |
| GGGGGAGGGGAAGGGGCGGGG | CCCCGCCCCTTCCCCTCCCCC |
| GGGGGCCTGGGTGGGAATGGG | CCCATTCCCACCCAGGCCCCC |
| GGGGGCGGGGCCGGGAAGGGG | CCCCTTCCCGGCCCCGCCCCC |
| GGGGGCTGAGGGAGGGCGGGG | CCCCGCCCTCCCTCAGCCCCC |
| GGGGGGGTGCTGGGTCTGGGG | CCCCAGACCCAGCACCCCCCC |
| GGGGGGTGGGAGGGAAGGGGG | CCCCCTTCCCTCCCACCCCCC |
| GGGGGGTGGGGTTGGGCGGGG | CCCCGCCCAACCCCACCCCCC |
| GGGGGGTTTGGGGTGGGAGGG | CCCTCCCACCCCAAACCCCCC |
| GGGGGTGGGGGCTGGGGGGGG | CCCCCCCCAGCCCCCACCCCC |
| GGGGGTGGGGGGGACAAAGGG | CCCTTTGTCCCCCCCACCCCC |
| GGGGGTGGGGGTGGGGTGGGG | CCCCACCCCACCCCCACCCCC |
| GGGGGTGGGGTTGGGATTGGG | CCCAATCCCAACCCCACCCCC |
| GGGGTCAGGGTGGAGGGTGGG | CCCACCCTCCACCCTGACCCC |
| GGGGTGCGGGCCCCGGGCGGG | CCCGCCCGGGGCCCGCACCCC |
| GGGGTGGGCAGGGGGCCAGGG | CCCTGGCCCCCTGCCCACCCC |
| GGGGTGGGGCAGGGCTGGGGG | CCCCCAGCCCTGCCCCACCCC |
| GGGGTTGGGTTGGGAGGAGGG | CCCTCCTCCCAACCCAACCCC |
| GGGTCAGGGTTGGGGACGGGG | CCCCGTCCCCAACCCTGACCC |
| GGGTGCGGGAGGCGGGCGGGG | CCCCGCCCGCCTCCCGCACCC |
| GGGTGCGGGTGAGGGTGTGGG | CCCACACCCTCACCCGCACCC |
| GGGTGGGAGGGGCACGGAGGG | CCCTCCGTGCCCCTCCCACCC |
| GGGTGGGATTGAGGGGCGGGG | CCCCGCCCCTCAATCCCACCC |
| GGGTGGGGAGGGAGGTGGGGG | CCCCCACCTCCCTCCCCACCC |
| GGGTGGGGGTGGGGGCTGGGG | CCCCAGCCCCCACCCCCACCC |
| GGGTTTGGGGAGGGACCTGGG | CCCAGGTCCCTCCCCAAACCC |
| CCCAAAGCCCACCCCAGCCCCC | GGGGGCTGGGGTGGGCTTTGGG |
| CCCAAATGCCCATTCCCTTCCC | GGGAAGGGAATGGGCATTTGGG |
| CCCAACCCCAGGCCCCCTTCCC | GGGAAGGGGGCCTGGGGTTGGG |
| CCCAACCCCCGTGCCCCCTCCC | GGGAGGGGGCACGGGGGTTGGG |
| CCCAATCTCCCTCCCCCACCCC | GGGGTGGGGGAGGGAGATTGGG |
| CCCACAGCCCTCACCCTCTCCC | GGGAGAGGGTGAGGGCTGTGGG |
| CCCACCCCCCGCCCCCTCCCCC | GGGGGAGGGGGCGGGGGGTGGG |
| CCCACCCTAACCCACCGCACCC | GGGTGCGGTGGGTTAGGGTGGG |
| CCCACCTCCCCGCGACCCGCCC | GGGCGGGTCGCGGGGAGGTGGG |
| CCCACGCGGGCCCTGCCCACCC | GGGTGGGCAGGGCCCGCGTGGG |
| CCCACGGCCCCTCCCTCTGCCC | GGGCAGAGGGAGGGGCCGTGGG |
| CCCAGATCCCCTTCCCTGACCC | GGGTCAGGGAAGGGGATCTGGG |
| CCCAGGACCCGCCCCCAGCCCC | GGGGCTGGGGGCGGGTCCTGGG |
| CCCAGGAGGCCCTCCCTTCCCC | GGGGAAGGGAGGGCCTCCTGGG |
| CCCCAACCCCTGTCCCTGACCC | GGGTCAGGGACAGGGGTTGGGG |
| CCCCAAGTCCCACCCCCGCCCC | GGGGCGGGGGTGGGACTTGGGG |
| CCCCACCCACCAGCCCCACCCC | GGGGTGGGGCTGGTGGGTGGGG |
| CCCCACCCCCCCCACAACCCCC | GGGGGTTGTGGGGGGGGTGGGG |
| CCCCACCCCCGCGCCCCAGCCC | GGGCTGGGGCGCGGGGGTGGGG |
| CCCCACCCGGCCCTCCTGCCCC | GGGGCAGGAGGGCCGGGTGGGG |
| CCCCAGCCCCAAACCCAGCCCC | GGGGCTGGGTTTGGGGCTGGGG |
| CCCCAGCCCCAGCCCCAGCCCC | GGGGCTGGGGCTGGGGCTGGGG |
| CCCCAGCCCCCGCCCACATCCC | GGGATGTGGGCGGGGGCTGGGG |
| CCCCAGCGCCCTCACCCGTCCC | GGGACGGGTGAGGGCGCTGGGG |
| CCCCATCCCCATCCCCACCCCC | GGGGGTGGGGATGGGGATGGGG |
| CCCCATCTGTCCCACCCATCCC | GGGATGGGTGGGACAGATGGGG |
| CCCCCACCCCAGGCCCTGCCCC | GGGGCAGGGCCTGGGGTGGGGG |
| CCCCCACGCCCCATTCCCTCCC | GGGAGGGAATGGGGCGTGGGGG |
| CCCCCAGCCCGGCCCCCGCCCC | GGGGCGGGGGCCGGGCTGGGGG |
| CCCCCCACCCACCCTGCACCCC | GGGGTGCAGGGTGGGTGGGGGG |
| CCCCCCACCCCAACCCCCACCC | GGGTGGGGGTTGGGGTGGGGGG |
| CCCCCCTCCCCCGCCCATCCCC | GGGGATGGGCGGGGGAGGGGGG |
| CCCCCGCCCCCACGCCCTGCCC | GGGCAGGGCGTGGGGGCGGGGG |
| CCCCCTCACCCCACCCTTCCCC | GGGGAAGGGTGGGGTGAGGGGG |
| CCCCGACCCCAGCCCGGGCCCC | GGGGCCCGGGCTGGGGTCGGGG |
| CCCCGACCCCTGGCCCTACCCC | GGGGTAGGGCCAGGGGTCGGGG |
| CCCCGCCCCGTCCCCCAGCCCC | GGGGCTGGGGGACGGGGCGGGG |
| CCCCGCCCCTTCCCTCCCTCCC | GGGAGGGAGGGAAGGGGCGGGG |
| CCCCGCCCGCGCCCCGCTCCCC | GGGGAGCGGGGCGCGGGCGGGG |
| CCCCGGCCCCGGCCCCGGCCCC | GGGGCCGGGGCCGGGGCCGGGG |
| CCCCGGCCCCGGCCCCGGTCCC | GGGACCGGGGCCGGGGCCGGGG |
| CCCCGTCCCCTCCCTCAGCCCC | GGGGCTGAGGGAGGGGACGGGG |
| CCCCGTCCCGGCCCAGACCCCC | GGGGGTCTGGGCCGGGACGGGG |
| CCCCTAACCCTTCCCAAACCCC | GGGGTTTGGGAAGGGTTAGGGG |
| CCCCTACCCATTCCCCCCACCC | GGGTGGGGGGAATGGGTAGGGG |
| CCCCTCCAGCCCACCCCTTCCC | GGGAAGGGGTGGGCTGGAGGGG |
| CCCCTCCCCCACCCCCATCCCC | GGGGATGGGGGTGGGGGAGGGG |
| CCCCTCCCTCTCTCCCGCCCCC | GGGGGCGGGAGAGAGGGAGGGG |
| CCCCTCCTCCCTCCCTCCGCCC | GGGCGGAGGGAGGGAGGAGGGG |
| CCCCTGCCCATCCCTCCCTCCC | GGGAGGGAGGGATGGGCAGGGG |
| CCCCTGGCGCCCGCGCCCACCC | GGGTGGGCGCGGGCGCCAGGGG |
| CCCCTTCACCCGCCCCTCGCCC | GGGCGAGGGGCGGGTGAAGGGG |
| CCCCTTCCCCTCCCTCCCCCCC | GGGGGGGAGGGAGGGGAAGGGG |
| CCCCTTCCTGCCCCTCCCCCCC | GGGGGGGAGGGGCAGGAAGGGG |
| CCCGCACCCACCTCCCGGCCCC | GGGGCCGGGAGGTGGGTGCGGG |
| CCCGCCCCAGCCCCCATCCCCC | GGGGGATGGGGGCTGGGGCGGG |
| CCCGCCCGCAGCCCGCCCTCCC | GGGAGGGCGGGCTGCGGGCGGG |
| CCCGCCTCCCCACGCCCAGCCC | GGGCTGGGCGTGGGGAGGCGGG |
| CCCGCGGCCCGCCCGGCCTCCC | GGGAGGCCGGGCGGGCCGCGGG |
| CCCGGCCCCGTGCCCGAAGCCC | GGGCTTCGGGCACGGGGCCGGG |
| CCCGTGCCCAAATTCCCCACCC | GGGTGGGGAATTTGGGCACGGG |
| CCCTACCCCCCACCCCTCTCCC | GGGAGAGGGGTGGGGGGTAGGG |
| CCCTACCCCTCCCCGTGACCCC | GGGGTCACGGGGAGGGGTAGGG |
| CCCTCCCCCGGCCCCCCGGCCC | GGGCCGGGGGGCCGGGGGAGGG |
| CCCTCCCCTCCCCGCCGTTCCC | GGGAACGGCGGGGAGGGGAGGG |
| CCCTCCCGCCCCCATAACCCCC | GGGGGTTATGGGGGCGGGAGGG |
| CCCTCCCTCCCCCAAGAACCCC | GGGGTTCTTGGGGGAGGGAGGG |
| CCCTCCCTCTCTCCCCGCCCCC | GGGGGCGGGGAGAGAGGGAGGG |
| CCCTCCCTGGGCCCTCCCACCC | GGGTGGGAGGGCCCAGGGAGGG |
| CCCTCCTTCCCACCCAACCCCC | GGGGGTTGGGTGGGAAGGAGGG |
| CCCTCTCCCCGCCTCCCACCCC | GGGGTGGGAGGCGGGGAGAGGG |
| CCCTCTCTATCCCTCCCCCCCC | GGGGGGGGAGGGATAGAGAGGG |
| CCCTGCCCACTCCCTGTGCCCC | GGGGCACAGGGAGTGGGCAGGG |
| CCCTGCCCCCATCCCCGCCCCC | GGGGGCGGGGATGGGGGCAGGG |
| CCCTGCCCCGCCCCCCAAGCCC | GGGCTTGGGGGGCGGGGCAGGG |
| CCCTGCGGCCCCGGCCCGCCCC | GGGGCGGGCCGGGGCCGCAGGG |
| CCCTGCTCCCTGCCCTGCCCCC | GGGGGCAGGGCAGGGAGCAGGG |
| CCCTGGAAGCCCCTCCCAACCC | GGGTTGGGAGGGGCTTCCAGGG |
| CCCTGGAGCCCCACCCAGCCCC | GGGGCTGGGTGGGGCTCCAGGG |
| CCCTGGCCCCCTCCCCCGCCCC | GGGGCGGGGGAGGGGGCCAGGG |
| CCCTGGCCCTGGCCCTGGCCCC | GGGGCCAGGGCCAGGGCCAGGG |
| CCCTTCCCAGCCCACCCGCCCC | GGGGCGGGTGGGCTGGGAAGGG |
| CCCTTTTCCCAAACCCAAGCCC | GGGCTTGGGTTTGGGAAAAGGG |
| GGGAAGTAGGGAGGGGTAGGGG | CCCCTACCCCTCCCTACTTCCC |
| GGGAATGGGGAGGACGGGAGGG | CCCTCCCGTCCTCCCCATTCCC |
| GGGACCCTGGGGGTGGGAGGGG | CCCCTCCCACCCCCAGGGTCCC |
| GGGACTGGGGGAGGGGAACGGG | CCCGTTCCCCTCCCCCAGTCCC |
| GGGAGCAGGGAGAGGGAGAGGG | CCCTCTCCCTCTCCCTGCTCCC |
| GGGAGGAGGGAAGGGAGAGGGG | CCCCTCTCCCTTCCCTCCTCCC |
| GGGAGGAGGGAGGGGAGGAGGG | CCCTCCTCCCCTCCCTCCTCCC |
| GGGAGGGAAGATGGGTGCTGGG | CCCAGCACCCATCTTCCCTCCC |
| GGGAGGGACAGAGAGGGGCGGG | CCCGCCCCTCTCTGTCCCTCCC |
| GGGAGGGACGCGGGGAGGGGGG | CCCCCCTCCCCGCGTCCCTCCC |
| GGGAGGGAGGGGGCGGCGGGGG | CCCCCGCCGCCCCCTCCCTCCC |
| GGGAGGGCGTAGAGGGCCAGGG | CCCTGGCCCTCTACGCCCTCCC |
| GGGAGGGGGCAGGGGCAGGGGG | CCCCCTGCCCCTGCCCCCTCCC |
| GGGAGGGGGGGAAGGGGCGGGG | CCCCGCCCCTTCCCCCCCTCCC |
| GGGAGGGGGTGGGGACAGAGGG | CCCTCTGTCCCCACCCCCTCCC |
| GGGAGGGGTGGGAAGAGGAGGG | CCCTCCTCTTCCCACCCCTCCC |
| GGGAGGGGTGGGGCAGGTAGGG | CCCTACCTGCCCCACCCCTCCC |
| GGGATGGGGTACGGGTTAGGGG | CCCCTAACCCGTACCCCATCCC |
| GGGCACAGGGAGGGGGAGGGGG | CCCCCTCCCCCTCCCTGTGCCC |
| GGGCAGAGGGAGGGGCCGTGGG | CCCACGGCCCCTCCCTCTGCCC |
| GGGCAGATGGGGCTGGGATGGG | CCCATCCCAGCCCCATCTGCCC |
| GGGCAGGGCCCTGGGGGAGGGG | CCCCTCCCCCAGGGCCCTGCCC |
| GGGCAGGTGGGAGGGAGGAGGG | CCCTCCTCCCTCCCACCTGCCC |
| GGGCCAACTGGGTGGGGTGGGG | CCCCACCCCACCCAGTTGGCCC |
| GGGCCCCGGGAGGGCGGTGGGG | CCCCACCGCCCTCCCGGGGCCC |
| GGGCCGGGGGCTGGGGGCCGGG | CCCGGCCCCCAGCCCCCGGCCC |
| GGGCCTGGGGATGGGCCTGGGG | CCCCAGGCCCATCCCCAGGCCC |
| GGGCGGGAGGCTGGGGAGGGGG | CCCCCTCCCCAGCCTCCCGCCC |
| GGGCGGGCGGGCCGCGGGCGGG | CCCGCCCGCGGCCCGCCCGCCC |
| GGGCGGGGCCCAGGGCTGGGGG | CCCCCAGCCCTGGGCCCCGCCC |
| GGGCGGGGGCTGGGGGCGGGGG | CCCCCGCCCCCAGCCCCCGCCC |
| GGGCGTGGGCAGGGCTTGTGGG | CCCACAAGCCCTGCCCACGCCC |
| GGGCTCAGGGATGGGTGTCGGG | CCCGACACCCATCCCTGAGCCC |
| GGGCTGGGAGGCAGGGCGGGGG | CCCCCGCCCTGCCTCCCAGCCC |
| GGGCTGGGAGGGTCGGATAGGG | CCCTATCCGACCCTCCCAGCCC |
| GGGCTGGGCGGGCCGAGGAGGG | CCCTCCTCGGCCCGCCCAGCCC |
| GGGCTGGGGGTTGGGGACAGGG | CCCTGTCCCCAACCCCCAGCCC |
| GGGGACAGGGGGTGGGAAGGGG | CCCCTTCCCACCCCCTGTCCCC |
| GGGGACTGAGGGAGGGCTGGGG | CCCCAGCCCTCCCTCAGTCCCC |
| GGGGAGGGAGGAGGGCGGGGGG | CCCCCCGCCCTCCTCCCTCCCC |
| GGGGAGGGCAGGGAGCAGGGGG | CCCCCTGCTCCCTGCCCTCCCC |
| GGGGAGGGGAGTGGGCAGTGGG | CCCACTGCCCACTCCCCTCCCC |
| GGGGAGGGGCAGGGGGCGGGGG | CCCCCGCCCCCTGCCCCTCCCC |
| GGGGAGGGGGCCCCTGGGAGGG | CCCTCCCAGGGGCCCCCTCCCC |
| GGGGAGGGGGGCGGGGAGGGGG | CCCCCTCCCCGCCCCCCTCCCC |
| GGGGATGGGGAAGGGGGCGGGG | CCCCGCCCCCTTCCCCATCCCC |
| GGGGCAAGGGCGAGGGCCTGGG | CCCAGGCCCTCGCCCTTGCCCC |
| GGGGCAGGGGCGGGGGGTTGGG | CCCAACCCCCCGCCCCTGCCCC |
| GGGGCAGGGTTGTGGGGAGGGG | CCCCTCCCCACAACCCTGCCCC |
| GGGGCCGGGGCGGGGATCCGGG | CCCGGATCCCCGCCCCGGCCCC |
| GGGGCCGGGGCTGGGGTCGGGG | CCCCGACCCCAGCCCCGGCCCC |
| GGGGCGGGAGCCGGGCTCAGGG | CCCTGAGCCCGGCTCCCGCCCC |
| GGGGCTAAAGGGGCGGGTAGGG | CCCTACCCGCCCCTTTAGCCCC |
| GGGGCTGGGGCTGGGCCTTGGG | CCCAAGGCCCAGCCCCAGCCCC |
| GGGGCTGGGGCTGGGGTCAGGG | CCCTGACCCCAGCCCCAGCCCC |
| GGGGGACCGGGCGGGCTGGGGG | CCCCCAGCCCGCCCGGTCCCCC |
| GGGGGAGGAGGGCAGGGCAGGG | CCCTGCCCTGCCCTCCTCCCCC |
| GGGGGCCGGGCCCGGGGGCGGG | CCCGCCCCCGGGCCCGGCCCCC |
| GGGGGGAGGGACTGGGGTCGGG | CCCGACCCCAGTCCCTCCCCCC |
| GGGGGGAGGGAGGAGGGAAGGG | CCCTTCCCTCCTCCCTCCCCCC |
| GGGGGTGGGGGAGGGAGAGGGG | CCCCTCTCCCTCCCCCACCCCC |
| GGGGTCATGGGGTTGGGCTGGG | CCCAGCCCAACCCCATGACCCC |
| GGGGTGAGGGGCGGGCAGAGGG | CCCTCTGCCCGCCCCTCACCCC |
| GGGGTGGCGGGGTGGGTGTGGG | CCCACACCCACCCCGCCACCCC |
| GGGGTGGGACCACGGGGCAGGG | CCCTGCCCCGTGGTCCCACCCC |
| GGGGTGGGAGGGCAGGGGTGGG | CCCACCCCTGCCCTCCCACCCC |
| GGGGTGGGCTGGGGTACCTGGG | CCCAGGTACCCCAGCCCACCCC |
| GGGGTGGGGCAGGGCCCTGGGG | CCCCAGGGCCCTGCCCCACCCC |
| GGGGTGGGGGAGGGCAGAGGGG | CCCCTCTGCCCTCCCCCACCCC |
| GGGGTTGGGGAGGGATGAAGGG | CCCTTCATCCCTCCCCAACCCC |
| GGGTAGGGGGATGGGAAGAGGG | CCCTCTTCCCATCCCCCTACCC |
| GGGTCGGGATCGGGGAGTGGGG | CCCCACTCCCCGATCCCGACCC |
| GGGTCTGAGGGTGGGGCAAGGG | CCCTTGCCCCACCCTCAGACCC |
| GGGTGAGGGACTGGGGCGGGGG | CCCCCGCCCCAGTCCCTCACCC |
| GGGTGCGGGGTAGGGGCCGGGG | CCCCGGCCCCTACCCCGCACCC |
| GGGTGCTGGGCCTGGGCCTGGG | CCCAGGCCCAGGCCCAGCACCC |
| GGGTGGCGGGACTCAGGGCGGG | CCCGCCCTGAGTCCCGCCACCC |
| GGGTGGGAAGTACAGGGGCGGG | CCCGCCCCTGTACTTCCCACCC |
| GGGTGGGGGGACTGGGTCTGGG | CCCAGACCCAGTCCCCCCACCC |
| GGGTGGGGGTGGGGGTATAGGG | CCCTATACCCCCACCCCCACCC |
| GGGTGGGGGTGGGGTTTCTGGG | CCCAGAAACCCCACCCCCACCC |
| GGGTGGGGTGGGGGGATGAGGG | CCCTCATCCCCCCACCCCACCC |
| CCCAAAATCCCAGTATCCCACCC | GGGTGGGATACTGGGATTTTGGG |
| CCCAAGCCCCCCACCCAGCCCCC | GGGGGCTGGGTGGGGGGCTTGGG |
| CCCAAGCCCGGCCCCTATTCCCC | GGGGAATAGGGGCCGGGCTTGGG |
| CCCACCCACCCGCCCGCCCTCCC | GGGAGGGCGGGCGGGTGGGTGGG |
| CCCACCCACCTGCCCTGCCTCCC | GGGAGGCAGGGCAGGTGGGTGGG |
| CCCACCCCCTACCCTGCCACCCC | GGGGTGGCAGGGTAGGGGGTGGG |
| CCCACCCGGCCCAGCCCGGCCCC | GGGGCCGGGCTGGGCCGGGTGGG |
| CCCACCCTCCACCCCTGCCCCCC | GGGGGGCAGGGGTGGAGGGTGGG |
| CCCACCGCCCCCGCCCCTTTCCC | GGGAAAGGGGCGGGGGCGGTGGG |
| CCCACTGTCTCCCCGCCCCTCCC | GGGAGGGGCGGGGAGACAGTGGG |
| CCCAGCCCCAGGCCCCTCTGCCC | GGGCAGAGGGGCCTGGGGCTGGG |
| CCCAGCCCCGGCCCAGCCCGCCC | GGGCGGGCTGGGCCGGGGCTGGG |
| CCCAGCCCGGCTCCCAGCCGCCC | GGGCGGCTGGGAGCCGGGCTGGG |
| CCCAGCCCTGCCCTGCCCTGCCC | GGGCAGGGCAGGGCAGGGCTGGG |
| CCCATCCCAGACCCAGTCCCCCC | GGGGGGACTGGGTCTGGGATGGG |
| CCCATCCCCGGAAGCCCATCCCC | GGGGATGGGCTTCCGGGGATGGG |
| CCCATGCCCCCTGATCCCACCCC | GGGGTGGGATCAGGGGGCATGGG |
| CCCCAATCCCCCACCCCCTGCCC | GGGCAGGGGGTGGGGGATTGGGG |
| CCCCACAGCCCCACCCCCTGCCC | GGGCAGGGGGTGGGGCTGTGGGG |
| CCCCACCCCACTCCACCCACCCC | GGGGTGGGTGGAGTGGGGTGGGG |
| CCCCACCCCGCTCCACCCACCCC | GGGGTGGGTGGAGCGGGGTGGGG |
| CCCCACCCGCTCTGGCCCCACCC | GGGTGGGGCCAGAGCGGGTGGGG |
| CCCCACCCTCTCCCTGAAGTCCC | GGGACTTCAGGGAGAGGGTGGGG |
| CCCCAGTGCCCCCTCCCTCTCCC | GGGAGAGGGAGGGGGCACTGGGG |
| CCCCCACCCCCACCCCACCTCCC | GGGAGGTGGGGTGGGGGTGGGGG |
| CCCCCACCCCTCCCAGTTCCCCC | GGGGGAACTGGGAGGGGTGGGGG |
| CCCCCCACCCCTCCCCGAGGCCC | GGGCCTCGGGGAGGGGTGGGGGG |
| CCCCCCCAGCCCCTCCCAGCCCC | GGGGCTGGGAGGGGCTGGGGGGG |
| CCCCCCCTCCCTCCCTTCTTCCC | GGGAAGAAGGGAGGGAGGGGGGG |
| CCCCCCTCGCCCGCCTCCCTCCC | GGGAGGGAGGCGGGCGAGGGGGG |
| CCCCCGCACCCACCCGGGGCCCC | GGGGCCCCGGGTGGGTGCGGGGG |
| CCCCCGCCCGATGTCCCAGCCCC | GGGGCTGGGACATCGGGCGGGGG |
| CCCCCGCCTCCCGGCCCCCTCCC | GGGAGGGGGCCGGGAGGCGGGGG |
| CCCCCTCACCCCCTCCCCTTCCC | GGGAAGGGGAGGGGGTGAGGGGG |
| CCCCCTCCCAGCCCCCCGGGCCC | GGGCCCGGGGGGCTGGGAGGGGG |
| CCCCCTCCCCCTGGGCCCACCCC | GGGGTGGGCCCAGGGGGAGGGGG |
| CCCCCTGCCCCACCCCCTTGCCC | GGGCAAGGGGGTGGGGCAGGGGG |
| CCCCGCCCAGCCCAGCCCAGCCC | GGGCTGGGCTGGGCTGGGCGGGG |
| CCCCGCCCCACCCCCTGCGGCCC | GGGCCGCAGGGGGTGGGGCGGGG |
| CCCCGCCCCTTCCCCCAAGCCCC | GGGGCTTGGGGGAAGGGGCGGGG |
| CCCCTACCCCCACCTTCCCTCCC | GGGAGGGAAGGTGGGGGTAGGGG |
| CCCCTCCCCATCCCACCCACCCC | GGGGTGGGTGGGATGGGGAGGGG |
| CCCCTCCCCCACCACCCCCTCCC | GGGAGGGGGTGGTGGGGGAGGGG |
| CCCCTCCCCCCATGCCCTGCCCC | GGGGCAGGGCATGGGGGGAGGGG |
| CCCCTCCCCGCCTGCCCCGCCCC | GGGGCGGGGCAGGCGGGGAGGGG |
| CCCCTCCCCTTCCCCCTCCCCCC | GGGGGGAGGGGGAAGGGGAGGGG |
| CCCCTCCCGCGTGAGCCCCACCC | GGGTGGGGCTCACGCGGGAGGGG |
| CCCCTCCCTCCCTCCCGCGGCCC | GGGCCGCGGGAGGGAGGGAGGGG |
| CCCCTCCTCCCTCCCCAGATCCC | GGGATCTGGGGAGGGAGGAGGGG |
| CCCCTGCCCATGCCCACTGCCCC | GGGGCAGTGGGCATGGGCAGGGG |
| CCCCTTACCCACCCTCCACCCCC | GGGGGTGGAGGGTGGGTAAGGGG |
| CCCCTTCCCAGTGCCCTCGTCCC | GGGACGAGGGCACTGGGAAGGGG |
| CCCCTTCCCCCTCCCGCCTCCCC | GGGGAGGCGGGAGGGGGAAGGGG |
| CCCCTTCCCCTCCCACGCTGCCC | GGGCAGCGTGGGAGGGGAAGGGG |
| CCCGAGCCCGCTCCCCCAGCCCC | GGGGCTGGGGGAGCGGGCTCGGG |
| CCCGCCACCCCTTCCCCCACCCC | GGGGTGGGGGAAGGGGTGGCGGG |
| CCCGCCCCCAGAGCCCCCTCCCC | GGGGAGGGGGCTCTGGGGGCGGG |
| CCCGCCCCGCCTCCCAGCCCCCC | GGGGGGCTGGGAGGCGGGGCGGG |
| CCCGCCTCCCCCACCCCACCCCC | GGGGGTGGGGTGGGGGAGGCGGG |
| CCCGCTCCCGCCCGCGCCGGCCC | GGGCCGGCGCGGGCGGGAGCGGG |
| CCCGGCCGCCCCACCCGCGACCC | GGGTCGCGGGTGGGGCGGCCGGG |
| CCCGGGCAGCCCGCGCCCCACCC | GGGTGGGGCGCGGGCTGCCCGGG |
| CCCGGGGCCCCTGAGCCCATCCC | GGGATGGGCTCAGGGGCCCCGGG |
| CCCTACCCCAAGACCCCTAGCCC | GGGCTAGGGGTCTTGGGGTAGGG |
| CCCTAGTCCCCTAGCCCAGCCCC | GGGGCTGGGCTAGGGGACTAGGG |
| CCCTCCCAGCCCCTCCTCCTCCC | GGGAGGAGGAGGGGCTGGGAGGG |
| CCCTCCCATCCCAGCCCCAGCCC | GGGCTGGGGCTGGGATGGGAGGG |
| CCCTCCCCCCCCTTCCCCCCCCC | GGGGGGGGGAAGGGGGGGGAGGG |
| CCCTCCCCTCTTCGCCCTCCCCC | GGGGGAGGGCGAAGAGGGGAGGG |
| CCCTCCCTCCACCCTCCTACCCC | GGGGTAGGAGGGTGGAGGGAGGG |
| CCCTCCGCCCGTCCCTGCTCCCC | GGGGAGCAGGGACGGGCGGAGGG |
| CCCTCGTCCCCACCCCCATCCCC | GGGGATGGGGGTGGGGACGAGGG |
| CCCTCTCCCCTCTCCCCAACCCC | GGGGTTGGGGAGAGGGGAGAGGG |
| CCCTGCCCATCCCCAACCACCCC | GGGGTGGTTGGGGATGGGCAGGG |
| CCCTGCCCTGCTCAGCCCAGCCC | GGGCTGGGCTGAGCAGGGCAGGG |
| CCCTGCTCCCCACCCTCACCCCC | GGGGGTGAGGGTGGGGAGCAGGG |
| CCCTTCACCCCTCCCCAATTCCC | GGGAATTGGGGAGGGGTGAAGGG |
| CCCTTCATCCCCCACCCCCACCC | GGGTGGGGGTGGGGGATGAAGGG |
| CCCTTTCCCATCCACCCCCACCC | GGGTGGGGGTGGATGGGAAAGGG |
| CCCTTTCTCCCCACCCCCACCCC | GGGGTGGGGGTGGGGAGAAAGGG |
| GGGAACCCGTGGGGGAGGGAGGG | CCCTCCCTCCCCCACGGGTTCCC |
| GGGAACTGGGGCTGGGGCTAGGG | CCCTAGCCCCAGCCCCAGTTCCC |
| GGGAAGGGGAAGGGGAGGAGGGG | CCCCTCCTCCCCTTCCCCTTCCC |
| GGGAAGTGGGTTGGGGGAAGGGG | CCCCTTCCCCCAACCCACTTCCC |
| GGGACACCCAGGGGAGGGAAGGG | CCCTTCCCTCCCCTGGGTGTCCC |
| GGGACTGGGGCGGGGGAGGGGGG | CCCCCCTCCCCCGCCCCAGTCCC |
| GGGAGCACGGGGTGGGGGTGGGG | CCCCACCCCCACCCCGTGCTCCC |
| GGGAGCAGCTGGGTGGGAATGGG | CCCATTCCCACCCAGCTGCTCCC |
| GGGAGCAGGGAGGGCCCCAGGGG | CCCCTGGGGCCCTCCCTGCTCCC |
| GGGAGGAGGGAGACGGGCTGGGG | CCCCAGCCCGTCTCCCTCCTCCC |
| GGGAGGGAAGTCTGGGTAAGGGG | CCCCTTACCCAGACTTCCCTCCC |
| GGGAGGGAATGGGGCGTGGGGGG | CCCCCCACGCCCCATTCCCTCCC |
| GGGAGGGAGGTTGCGGGGAGGGG | CCCCTCCCCGCAACCTCCCTCCC |
| GGGAGGGATGCGAGGGGGTGGGG | CCCCACCCCCTCGCATCCCTCCC |
| GGGAGGGCCAAGGGGATGATGGG | CCCATCATCCCCTTGGCCCTCCC |
| GGGAGGGGCAGAGAGGGCCAGGG | CCCTGGCCCTCTCTGCCCCTCCC |
| GGGAGGGGCCGAGGGGGTGAGGG | CCCTCACCCCCTCGGCCCCTCCC |
| GGGAGGGGCTGGGCAGGGCGGGG | CCCCGCCCTGCCCAGCCCCTCCC |
| GGGAGGGGGGCAGGGGAGTGGGG | CCCCACTCCCCTGCCCCCCTCCC |
| GGGAGGGGGTCAGGGAAACGGGG | CCCCGTTTCCCTGACCCCCTCCC |
| GGGAGGGTTGGAGGGGAGAAGGG | CCCTTCTCCCCTCCAACCCTCCC |
| GGGAGGTGGGAGGTGGGAGGGGG | CCCCCTCCCACCTCCCACCTCCC |
| GGGCAGAGGGCCGGCGGGCAGGG | CCCTGCCCGCCGGCCCTCTGCCC |
| GGGCAGGGGATGGGGCATCAGGG | CCCTGATGCCCCATCCCCTGCCC |
| GGGCAGTGGGCCGAGGGAGTGGG | CCCACTCCCTCGGCCCACTGCCC |
| GGGCCAAGAAGGGCAGGGGTGGG | CCCACCCCTGCCCTTCTTGGCCC |
| GGGCCAGCGGGCCGATGGGTGGG | CCCACCCATCGGCCCGCTGGCCC |
| GGGCCCGGGGCTCGCGGGAGGGG | CCCCTCCCGCGAGCCCCGGGCCC |
| GGGCCTGGGTGGCAGGGGGTGGG | CCCACCCCCTGCCACCCAGGCCC |
| GGGCGAGCGGGCCAGGGGCCGGG | CCCGGCCCCTGGCCCGCTCGCCC |
| GGGCGAGTGGGGGCCGGGCGGGG | CCCCGCCCGGCCCCCACTCGCCC |
| GGGCGCCGGGCAGGGCCGGCGGG | CCCGCCGGCCCTGCCCGGCGCCC |
| GGGCGGCCGGGGGGGTCTCTGGG | CCCAGAGACCCCCCCGGCCGCCC |
| GGGCGGGAGGGAATCGGGGAGGG | CCCTCCCCGATTCCCTCCCGCCC |
| GGGCGGGCGGGCGGGTGAGGGGG | CCCCCTCACCCGCCCGCCCGCCC |
| GGGCGGGCGGGGAGGGGGAGGGG | CCCCTCCCCCTCCCCGCCCGCCC |
| GGGCTGGGAGGAGGGTGTGAGGG | CCCTCACACCCTCCTCCCAGCCC |
| GGGCTGGGATCCACGGGGAAGGG | CCCTTCCCCGTGGATCCCAGCCC |
| GGGCTGGGGGGAAGGGGCAAGGG | CCCTTGCCCCTTCCCCCCAGCCC |
| GGGGAAGGGAGGGCAGGGTGGGG | CCCCACCCTGCCCTCCCTTCCCC |
| GGGGAGGGGAAGCCGGGGGCGGG | CCCGCCCCCGGCTTCCCCTCCCC |
| GGGGAGGGGCGGGGTCGGCTGGG | CCCAGCCGACCCCGCCCCTCCCC |
| GGGGAGGGGGTGGGGGAGGAGGG | CCCTCCTCCCCCACCCCCTCCCC |
| GGGGCAGGGGGCCTGGGGTGGGG | CCCCACCCCAGGCCCCCTGCCCC |
| GGGGCAGGGGGCGGGGCGGAGGG | CCCTCCGCCCCGCCCCCTGCCCC |
| GGGGCCCGGGTCGGGACGGAGGG | CCCTCCGTCCCGACCCGGGCCCC |
| GGGGCCGGGCAGAGGGCGCGGGG | CCCCGCGCCCTCTGCCCGGCCCC |
| GGGGCCGGGGTCGGGCGGGCGGG | CCCGCCCGCCCGACCCCGGCCCC |
| GGGGCGCAGGGGGGCGGGGAGGG | CCCTCCCGCCCCCCCTGCGCCCC |
| GGGGCGGGGACAGAGGGAAGGGG | CCCCTTCCCTCTGTCCCCGCCCC |
| GGGGCGGGGAGGGGCTGGGCGGG | CCCGCCCAGCCCCTCCCCGCCCC |
| GGGGCGGGGGCGGGCGCCGCGGG | CCCGCGGCGCCCGCCCCCGCCCC |
| GGGGCTAAGGGCCGGGGTGGGGG | CCCCCACCCCGGCCCTTAGCCCC |
| GGGGCTGCGGGCATGGGGCCGGG | CCCGGCCCCATGCCCGCAGCCCC |
| GGGGCTGGAGGGAGGGGGTGGGG | CCCCACCCCCTCCCTCCAGCCCC |
| GGGGCTGGGAAGGGGCGGAGGGG | CCCCTCCGCCCCTTCCCAGCCCC |
| GGGGCTGGGAGTGGGGAGGAGGG | CCCTCCTCCCCACTCCCAGCCCC |
| GGGGGGCTGGGGAGGGGGGTGGG | CCCACCCCCCTCCCCAGCCCCCC |
| GGGGGGGGGGGCGGGTAGAGGGG | CCCCTCTACCCGCCCCCCCCCCC |
| GGGGGGTGGGGGGGCTGCGCGGG | CCCGCGCAGCCCCCCCACCCCCC |
| GGGGGTAGGGGTGAAAGGGGGGG | CCCCCCCTTTCACCCCTACCCCC |
| GGGGGTGCTGGGGCTGGGAGGGG | CCCCTCCCAGCCCCAGCACCCCC |
| GGGGGTGGGCGGAGGGCGCCGGG | CCCGGCGCCCTCCGCCCACCCCC |
| GGGGGTGGGGAGGGGACAGAGGG | CCCTCTGTCCCCTCCCCACCCCC |
| GGGGGTGGGGGCTGGGATGGGGG | CCCCCATCCCAGCCCCCACCCCC |
| GGGGGTGGGGGGAGGGGAATGGG | CCCATTCCCCTCCCCCCACCCCC |
| GGGGTCGAGGGAAGGGGAGGGGG | CCCCCTCCCCTTCCCTCGACCCC |
| GGGGTCGGGGAGGGTACACGGGG | CCCCCTGTACCCTCCCCGACCCC |
| GGGGTGGGCTGGGCTGGGTGGGG | CCCCACCCAGCCCAGCCCACCCC |
| GGGGTGGGGGCGGGGGAGTCGGG | CCCGACTCCCCCGCCCCCACCCC |
| GGGGTGGGGGGGTGGGGGAAGGG | CCCTTCCCCCACCCCCCCACCCC |
| GGGGTGGGGGTGGGGAACTTGGG | CCCAAGTTCCCCACCCCCACCCC |
| GGGGTGGGTGGAGCGGGGTGGGG | CCCCACCCCGCTCCACCCACCCC |
| GGGGTGGGTGGGGGGTAAGGGGG | CCCCCTTACCCCCCACCCACCCC |
| GGGGTGTGGGAGTGGGGTGGGGG | CCCCCACCCCACTCCCACACCCC |
| GGGGTTGGGGGGGTGGGTGGGGG | CCCCCACCCACCCCCCCAACCCC |
| GGGTGACGGGCTGGGGGAGGGGG | CCCCCTCCCCCAGCCCGTCACCC |
| GGGTGGGACGCGTGGGGGAAGGG | CCCTTCCCCCACGCGTCCCACCC |
| GGGTGGGAGGGCTAGGGAAAGGG | CCCTTTCCCTAGCCCTCCCACCC |
| GGGTGGGGCAGGGTGGGGTGGGG | CCCCACCCCACCCTGCCCCACCC |
| GGGTGGGGCTGGGCGCGGCCGGG | CCCGGCCGCGCCCAGCCCCACCC |
| GGGTGGGGGGCGGGCGCCTGGGG | CCCCAGGCGCCCGCCCCCCACCC |
| GGGTGGGGTAGGGATCGGGAGGG | CCCTCCCGATCCCTACCCCACCC |
| GGGTGGGGTGGGAAGGGGGCGGG | CCCGCCCCCTTCCCACCCCACCC |
| GGGTGGGTGTGGGCATGAGGGGG | CCCCCTCATGCCCACACCCACCC |
| GGGTTAGGGGGTTGGGAGCTGGG | CCCAGCTCCCAACCCCCTAACCC |
| GGGTTGGGATCGAGGGTGAGGGG | CCCCTCACCCTCGATCCCAACCC |
| GGGTTGGGGGGGAAGGGGTAGGG | CCCTACCCCTTCCCCCCCAACCC |
| CCCACATCCCTCCATCCCTCTCCC | GGGAGAGGGATGGAGGGATGTGGG |
| CCCACCCACCCTCCCACACCTCCC | GGGAGGTGTGGGAGGGTGGGTGGG |
| CCCACCCGCTCTCCCCCCCAACCC | GGGTTGGGGGGGAGAGCGGGTGGG |
| CCCACCCTCCCGCCCCCTCAGCCC | GGGCTGAGGGGGCGGGAGGGTGGG |
| CCCACCCTCCGCCCGCCCTCTCCC | GGGAGAGGGCGGGCGGAGGGTGGG |
| CCCACCCTGCCCCCCACCCCACCC | GGGTGGGGTGGGGGGCAGGGTGGG |
| CCCACCCTGTCCCCAGGAAATCCC | GGGATTTCCTGGGGACAGGGTGGG |
| CCCACTCCCAACTGCCCTAAACCC | GGGTTTAGGGCAGTTGGGAGTGGG |
| CCCACTCCCCCTACCCGAGTCCCC | GGGGACTCGGGTAGGGGGAGTGGG |
| CCCAGACCCCGGCCCTCGCCTCCC | GGGAGGCGAGGGCCGGGGTCTGGG |
| CCCAGCGCTCCCTGTCCCCGCCCC | GGGGCGGGGACAGGGAGCGCTGGG |
| CCCAGCTACCCAGGCCCGTTGCCC | GGGCAACGGGCCTGGGTAGCTGGG |
| CCCAGGCCCCACGCCCTTGCACCC | GGGTGCAAGGGCGTGGGGCCTGGG |
| CCCAGGCCCTGGCGGCCCAGGCCC | GGGCCTGGGCCGCCAGGGCCTGGG |
| CCCATCCCACGGGCCCCACACCCC | GGGGTGTGGGGCCCGTGGGATGGG |
| CCCATGCCCTGCGGCCCCGGCCCC | GGGGCCGGGGCCGCAGGGCATGGG |
| CCCATTCCCGACCCTCTGCTTCCC | GGGAAGCAGAGGGTCGGGAATGGG |
| CCCCAACCCCGGCCCCCGCGCCCC | GGGGCGCGGGGGCCGGGGTTGGGG |
| CCCCAAGGCCCACCTCCCAGGCCC | GGGCCTGGGAGGTGGGCCTTGGGG |
| CCCCACAGCTCCCAACCCCCTCCC | GGGAGGGGGTTGGGAGCTGTGGGG |
| CCCCACCCCCCGGTCCCCCTCCCC | GGGGAGGGGGACCGGGGGGTGGGG |
| CCCCACCCCCTAGCTTCCCTCCCC | GGGGAGGGAAGCTAGGGGGTGGGG |
| CCCCACCCCTAGTCTGCCCTCCCC | GGGGAGGGCAGACTAGGGGTGGGG |
| CCCCACCTGCCCGCTCACCCTCCC | GGGAGGGTGAGCGGGCAGGTGGGG |
| CCCCACTCCCCTTCCCCCAGCCCC | GGGGCTGGGGGAAGGGGAGTGGGG |
| CCCCACTCCCCTTGCCCCACACCC | GGGTGTGGGGCAAGGGGAGTGGGG |
| CCCCAGCCCCCTCACCCTCCTCCC | GGGAGGAGGGTGAGGGGGCTGGGG |
| CCCCAGTCCCCTACCCGGATCCCC | GGGGATCCGGGTAGGGGACTGGGG |
| CCCCATCTCACCCCGCCCTCACCC | GGGTGAGGGCGGGGTGAGATGGGG |
| CCCCATGACGCCCCGCCCAAGCCC | GGGCTTGGGCGGGGCGTCATGGGG |
| CCCCCAGGACCCCGCCCCCGCCCC | GGGGCGGGGGCGGGGTCCTGGGGG |
| CCCCCATCCCGCCCCACCCAACCC | GGGTTGGGTGGGGCGGGATGGGGG |
| CCCCCCAAGGCCCCGGCCCCTCCC | GGGAGGGGCCGGGGCCTTGGGGGG |
| CCCCCCACCCACCCAGCCCTCCCC | GGGGAGGGCTGGGTGGGTGGGGGG |
| CCCCCCACCCCCCAACGCCCTCCC | GGGAGGGCGTTGGGGGGTGGGGGG |
| CCCCCCCCACCACCCCACCACCCC | GGGGTGGTGGGGTGGTGGGGGGGG |
| CCCCCCGCCCTCCCGCGCCGGCCC | GGGCCGGCGCGGGAGGGCGGGGGG |
| CCCCCCTCCCTCCCTCCGCTGCCC | GGGCAGCGGAGGGAGGGAGGGGGG |
| CCCCCGCCCATCCCCTCCCCGCCC | GGGCGGGGAGGGGATGGGCGGGGG |
| CCCCCGGCCCTGCCTCCCAAACCC | GGGTTTGGGAGGCAGGGCCGGGGG |
| CCCCCTCCCCGGCTGCCCTCCCCC | GGGGGAGGGCAGCCGGGGAGGGGG |
| CCCCCTGCCCAGACCCGGAGCCCC | GGGGCTCCGGGTCTGGGCAGGGGG |
| CCCCGCCCCAGCCCGCCCCGCCCC | GGGGCGGGGCGGGCTGGGGCGGGG |
| CCCCGCCCCTCTCCCGCTGCTCCC | GGGAGCAGCGGGAGAGGGGGCGGG |
| CCCCGCCCGCCGGCCCGCCGCCCC | GGGGCGGCGGGCCGGCGGGCGGGG |
| CCCCGCGCCCACCCCCATCGCCCC | GGGGCGATGGGGGTGGGCGCGGGG |
| CCCCGCGCCCCGGCCCTGCCGCCC | GGGCGGCAGGGCCGGGGCGCGGGG |
| CCCCGGCCCGCGCCCCTGGCCCCC | GGGGGCCAGGGGCGCGGGCCGGGG |
| CCCCGGGCCCAGGTCCCCCGCCCC | GGGGCGGGGGACCTGGGCCCGGGG |
| CCCCGGTAACCCCCGGTCCCTCCC | GGGAGGGACCGGGGGTTACCGGGG |
| CCCCTCAGCCCCACCCATAATCCC | GGGATTATGGGTGGGGCTGAGGGG |
| CCCCTCCCCACCCCATGCAGCCCC | GGGGCTGCATGGGGTGGGGAGGGG |
| CCCCTCCCCCAGCCCGCCGACCCC | GGGGTCGGCGGGCTGGGGGAGGGG |
| CCCCTCCCCCCGCCCTGCACCCCC | GGGGGTGCAGGGCGGGGGGAGGGG |
| CCCCTCCCCCGACCACCCCGCCCC | GGGGCGGGGTGGTCGGGGGAGGGG |
| CCCCTCCCCCTCCCAGCAAAGCCC | GGGCTTTGCTGGGAGGGGGAGGGG |
| CCCCTCCCCCTCCTCCCCTCCCCC | GGGGGAGGGGAGGAGGGGGAGGGG |
| CCCCTCCCCTCCCTCCCTCCTCCC | GGGAGGAGGGAGGGAGGGGAGGGG |
| CCCCTCCCTCCCACCCCCCACCCC | GGGGTGGGGGGTGGGAGGGAGGGG |
| CCCCTCCGGCCCGACCCCTCCCCC | GGGGGAGGGGTCGGGCCGGAGGGG |
| CCCCTCCTCTCCCGGGCCCCTCCC | GGGAGGGGCCCGGGAGAGGAGGGG |
| CCCCTGCCCATCCCCACTCCCCCC | GGGGGGAGTGGGGATGGGCAGGGG |
| CCCCTGGGGCCCTGCCCGTCACCC | GGGTGACGGGCAGGGCCCCAGGGG |
| CCCCTTCCCTATCCAGCCCGCCCC | GGGGCGGGCTGGATAGGGAAGGGG |
| CCCCTTGACCCCTCCCCACACCCC | GGGGTGTGGGGAGGGGTCAAGGGG |
| CCCCTTTCACCCCATCCCCTGCCC | GGGCAGGGGATGGGGTGAAAGGGG |
| CCCCTTTGCCCCCACCCCTTCCCC | GGGGAAGGGGTGGGGGCAAAGGGG |
| CCCGCAGCCCCCGGCCCCTCCCCC | GGGGGAGGGGCCGGGGGCTGCGGG |
| CCCGCAGGAACCCCACCCCTTCCC | GGGAAGGGGTGGGGTTCCTGCGGG |
| CCCGCCCCCCCCCGCCCCCACCCC | GGGGTGGGGGCGGGGGGGGGCGGG |
| CCCGCCCCTCTAAGCCCGGAGCCC | GGGCTCCGGGCTTAGAGGGGCGGG |
| CCCGCCTCGCCCCCACCCCCACCC | GGGTGGGGGTGGGGGCGAGGCGGG |
| CCCGCCTTCCCCATTGCCCGCCCC | GGGGCGGGCAATGGGGAAGGCGGG |
| CCCGCGCGCCCCACTGCCCTCCCC | GGGGAGGGCAGTGGGGCGCGCGGG |
| CCCGCTCGCTCCCTGCCCACTCCC | GGGAGTGGGCAGGGAGCGAGCGGG |
| CCCGGCCCAGCCCCTGCCCCTCCC | GGGAGGGGCAGGGGCTGGGCCGGG |
| CCCGGCCCCCTCGCCTCCCCTCCC | GGGAGGGGAGGCGAGGGGGCCGGG |
| CCCGGGACCCCCGCCCTCTCGCCC | GGGCGAGAGGGCGGGGGTCCCGGG |
| CCCTCACCCCTTCCCTTCCCACCC | GGGTGGGAAGGGAAGGGGTGAGGG |
| CCCTCACCCTGAGTCCCGTCACCC | GGGTGACGGGACTCAGGGTGAGGG |
| CCCTCCACTCCCTCCCTGACCCCC | GGGGGTCAGGGAGGGAGTGGAGGG |
| CCCTCCCCTGTCTCCCTCCTCCCC | GGGGAGGAGGGAGACAGGGGAGGG |
| CCCTCCCTCAGCCCCTTCCCCCCC | GGGGGGGAAGGGGCTGAGGGAGGG |
| CCCTCCCTCCTTCCCTCTTTCCCC | GGGGAAAGAGGGAAGGAGGGAGGG |
| CCCTCCCTCTTCCCACAGTCCCCC | GGGGGACTGTGGGAAGAGGGAGGG |
| CCCTCCCTCTTCTTCCCAACCCCC | GGGGGTTGGGAAGAAGAGGGAGGG |
| CCCTCCTACCCCCACCCAATACCC | GGGTATTGGGTGGGGGTAGGAGGG |
| CCCTCCTCCCACCCCCGCCTTCCC | GGGAAGGCGGGGGTGGGAGGAGGG |
| CCCTCGCACCCCGACCCCGCCCCC | GGGGGCGGGGTCGGGGTGCGAGGG |
| CCCTCGCCCAGACAGTCCCATCCC | GGGATGGGACTGTCTGGGCGAGGG |
| CCCTCTCTTCCCCACCCGGGGCCC | GGGCCCCGGGTGGGGAAGAGAGGG |
| CCCTGCACCCCAACCCTGCAGCCC | GGGCTGCAGGGTTGGGGTGCAGGG |
| CCCTGCCCTCCCCACCCCCTTCCC | GGGAAGGGGGTGGGGAGGGCAGGG |
| CCCTGGACTCCCCTCCCTCCTCCC | GGGAGGAGGGAGGGGAGTCCAGGG |
| CCCTGGCCCCAGGATCCCGAGCCC | GGGCTCGGGATCCTGGGGCCAGGG |
| CCCTGTCCCCTACTTCCCAACCCC | GGGGTTGGGAAGTAGGGGACAGGG |
| CCCTGTGGGTCCCAGACCCTCCCC | GGGGAGGGTCTGGGACCCACAGGG |
| CCCTGTTCCCAGGAAACCCTCCCC | GGGGAGGGTTTCCTGGGAACAGGG |
| CCCTTACCCCATCCCCACTGTCCC | GGGACAGTGGGGATGGGGTAAGGG |
| CCCTTCCCTCCCTTCCCCCTGCCC | GGGCAGGGGGAAGGGAGGGAAGGG |
| CCCTTCTGCCCACCCCTATCTCCC | GGGAGATAGGGGTGGGCAGAAGGG |
| CCCTTGTCCCCACCCCCAGCCCCC | GGGGGCTGGGGGTGGGGACAAGGG |
| CCCTTTTCCCCACCCCCCACCCCC | GGGGGTGGGGGGTGGGGAAAAGGG |
| CCCTTTTTTACCCCACCCTACCCC | GGGGTAGGGTGGGGTAAAAAAGGG |
| GGGAAAGGGCCAGAGGGCAAAGGG | CCCTTTGCCCTCTGGCCCTTTCCC |
| GGGAAGGGGACGGGACGTGCTGGG | CCCAGCACGTCCCGTCCCCTTCCC |
| GGGAAGTGGTGGGGGCGGGGAGGG | CCCTCCCCGCCCCCACCACTTCCC |
| GGGACTGGGGCCTGTAGGGTGGGG | CCCCACCCTACAGGCCCCAGTCCC |
| GGGAGAAGGGCGTGGGAGGGAGGG | CCCTCCCTCCCACGCCCTTCTCCC |
| GGGAGACTGGGTGGGGAGCCAGGG | CCCTGGCTCCCCACCCAGTCTCCC |
| GGGAGAGAAGGGTTGGGGGAAGGG | CCCTTCCCCCAACCCTTCTCTCCC |
| GGGAGATGGGCGGGGGGAGGGGGG | CCCCCCTCCCCCCGCCCATCTCCC |
| GGGAGCCAGGGGAGGGGGCTGGGG | CCCCAGCCCCCTCCCCTGGCTCCC |
| GGGAGGGAGGGAGGGAGTCCGGGG | CCCCGGACTCCCTCCCTCCCTCCC |
| GGGAGGGGAGGTGGGCTGGGCGGG | CCCGCCCAGCCCACCTCCCCTCCC |
| GGGATGGGGGGTGGGGGTGCTGGG | CCCAGCACCCCCACCCCCCATCCC |
| GGGATGTGGGAAGGGTTGTGGGGG | CCCCCACAACCCTTCCCACATCCC |
| GGGCACTGGGTGGGTGAGGCTGGG | CCCACCCTCACCCACCCAGTGCCC |
| GGGCAGAGCGGGGCGGGCTAAGGG | CCCTTAGCCCGCCCCGCTCTGCCC |
| GGGCAGCCTGGGCTGGGGCCAGGG | CCCTGGCCCCAGCCCAGGCTGCCC |
| GGGCAGGGCAGGGGCCCGGCAGGG | CCCTGCCGGGCCCCTGCCCTGCCC |
| GGGCAGGGCCAGGGTCCAGCAGGG | CCCTGCTGGACCCTGGCCCTGCCC |
| GGGCATGGTGGGGCAGGGCAGGGG | CCCCTGCCCTGCCCCACCATGCCC |
| GGGCCCACAGGGCCTGGGGCGGGG | CCCCGCCCCAGGCCCTGTGGGCCC |
| GGGCCCCGGGCTGGAGGGGCCGGG | CCCGGCCCCTCCAGCCCGGGGCCC |
| GGGCCCTGGGGAGGGGGCGGCGGG | CCCGCCGCCCCCTCCCCAGGGCCC |
| GGGCCGAAAAGGGGAGGGGGAGGG | CCCTCCCCCTCCCCTTTTCGGCCC |
| GGGCCGGGCCCTGGGCCAACGGGG | CCCCGTTGGCCCAGGGCCCGGCCC |
| GGGCCGGGCGTGGGGGCCTGTGGG | CCCACAGGCCCCCACGCCCGGCCC |
| GGGCCGGGCTGGGGGCACGGCGGG | CCCGCCGTGCCCCCAGCCCGGCCC |
| GGGCCTACGGGGAGGGGGTGGGGG | CCCCCACCCCCTCCCCGTAGGCCC |
| GGGCCTGAGGGTGGGCTGGGAGGG | CCCTCCCAGCCCACCCTCAGGCCC |
| GGGCCTGGGCTGGGGGGCCTGGGG | CCCCAGGCCCCCCAGCCCAGGCCC |
| GGGCCTGGGGCAGGGTGTGAGGGG | CCCCTCACACCCTGCCCCAGGCCC |
| GGGCCTGGGGGGCCGGGGCCTGGG | CCCAGGCCCCGGCCCCCCAGGCCC |
| GGGCGAGGGGGTCGGGAGGGTGGG | CCCACCCTCCCGACCCCCTCGCCC |
| GGGCGAGGGTCAGGGGGCGGTGGG | CCCACCGCCCCCTGACCCTCGCCC |
| GGGCGGAGGGTAGTGGGATGGGGG | CCCCCATCCCACTACCCTCCGCCC |
| GGGCGGCGGGCCCCGCGGGAAGGG | CCCTTCCCGCGGGGCCCGCCGCCC |
| GGGCGGGATTTAGGGCACAGTGGG | CCCACTGTGCCCTAAATCCCGCCC |
| GGGCGGGGACACGGAGGGTGGGGG | CCCCCACCCTCCGTGTCCCCGCCC |
| GGGCTCGGGGTGGGGGTGCTGGGG | CCCCAGCACCCCCACCCCGAGCCC |
| GGGCTCTAGGGAGACAGGGAGGGG | CCCCTCCCTGTCTCCCTAGAGCCC |
| GGGCTGGGAGGGAGGGGCGGGGGG | CCCCCCGCCCCTCCCTCCCAGCCC |
| GGGCTGGGCTGGGGGAGGGCAGGG | CCCTGCCCTCCCCCAGCCCAGCCC |
| GGGCTGTGGGGCTGGGCCCCAGGG | CCCTGGGGCCCAGCCCCACAGCCC |
| GGGGAAAGGGGAAGGGTGGGTGGG | CCCACCCACCCTTCCCCTTTCCCC |
| GGGGAAGGGCTGGGGTGGAGGGGG | CCCCCTCCACCCCAGCCCTTCCCC |
| GGGGAAGGGGGGAGGGCAGGAGGG | CCCTCCTGCCCTCCCCCCTTCCCC |
| GGGGAAGGGGTGCGTGGGCAGGGG | CCCCTGCCCACGCACCCCTTCCCC |
| GGGGAGAAGGGGGTCCGGGAGGGG | CCCCTCCCGGACCCCCTTCTCCCC |
| GGGGAGGGAGCTTGGGGTAAGGGG | CCCCTTACCCCAAGCTCCCTCCCC |
| GGGGAGGGGCGGGGCGGGGGAGGG | CCCTCCCCCGCCCCGCCCCTCCCC |
| GGGGAGGGGGCGGGGCTAGCTGGG | CCCAGCTAGCCCCGCCCCCTCCCC |
| GGGGAGTGGGGAGTGGGTGGGGGG | CCCCCCACCCACTCCCCACTCCCC |
| GGGGATGGGGGCGGGGGACCTGGG | CCCAGGTCCCCCGCCCCCATCCCC |
| GGGGCAGGGAGGGGCTGGGCTGGG | CCCAGCCCAGCCCCTCCCTGCCCC |
| GGGGCAGGGCAGGTGGGGTTGGGG | CCCCAACCCCACCTGCCCTGCCCC |
| GGGGCAGGGGCCTACGGGGAGGGG | CCCCTCCCCGTAGGCCCCTGCCCC |
| GGGGCAGGGGTCTCGGGCGCGGGG | CCCCGCGCCCGAGACCCCTGCCCC |
| GGGGCCCCCGGGTAGGGTGGCGGG | CCCGCCACCCTACCCGGGGGCCCC |
| GGGGCCTGCGGGTGCCGGGGCGGG | CCCGCCCCGGCACCCGCAGGCCCC |
| GGGGCGCGCGGGCCGGGGGCGGGG | CCCCGCCCCCGGCCCGCGCGCCCC |
| GGGGCGGGAGGGGGACGGGGCGGG | CCCGCCCCGTCCCCCTCCCGCCCC |
| GGGGCGGGGCTGGGGAGGTCGGGG | CCCCGACCTCCCCAGCCCCGCCCC |
| GGGGCTGGGGGAGGGTTCACAGGG | CCCTGTGAACCCTCCCCCAGCCCC |
| GGGGGAGGGGCGGCGGGGGCGGGG | CCCCGCCCCCGCCGCCCCTCCCCC |
| GGGGGAGGGGGGTGGGTCAGAGGG | CCCTCTGACCCACCCCCCTCCCCC |
| GGGGGCCGTGGGAGCGGGAAGGGG | CCCCTTCCCGCTCCCACGGCCCCC |
| GGGGGCTACGGGTCGGGGGAGGGG | CCCCTCCCCCGACCCGTAGCCCCC |
| GGGGGGAGGGAAGGGAGCCAAGGG | CCCTTGGCTCCCTTCCCTCCCCCC |
| GGGGGGCGGGGGCCGGGGCCCGGG | CCCGGGCCCCGGCCCCCGCCCCCC |
| GGGGGGCGGGGGCGGGGGCGGGGG | CCCCCGCCCCCGCCCCCGCCCCCC |
| GGGGGGGAGGGGGGAGGAGTTGGG | CCCAACTCCTCCCCCCTCCCCCCC |
| GGGGGGGCGACAGGGCGCTCGGGG | CCCCGAGCGCCCTGTCGCCCCCCC |
| GGGGGGGGCCACCAGGGTTTGGGG | CCCCAAACCCTGGTGGCCCCCCCC |
| GGGGGGTGGGGAGGGGTGGGTGGG | CCCACCCACCCCTCCCCACCCCCC |
| GGGGTCCCGGGGGCGGGGCCGGGG | CCCCGGCCCCGCCCCCGGGACCCC |
| GGGGTGAGGGGCCTTGGGGAGGGG | CCCCTCCCCAAGGCCCCTCACCCC |
| GGGGTGGGGAAGGGTGTGGAGGGG | CCCCTCCACACCCTTCCCCACCCC |
| GGGGTGGGGAGGGAGGGAAAAGGG | CCCTTTTCCCTCCCTCCCCACCCC |
| GGGGTGGGGGATGGGAGGGAGGGG | CCCCTCCCTCCCATCCCCCACCCC |
| GGGGTGGGGGGTGGGACTAGAGGG | CCCTCTAGTCCCACCCCCCACCCC |
| GGGGTGGGGTGGGGGAGGGACGGG | CCCGTCCCTCCCCCACCCCACCCC |
| GGGGTTAGGGATTTGCGGGGGGGG | CCCCCCCCGCAAATCCCTAACCCC |
| GGGTAGGGGGGAAGGGAAGGAGGG | CCCTCCTTCCCTTCCCCCCTACCC |
| GGGTCAGGGAAGTTGGGATGGGGG | CCCCCATCCCAACTTCCCTGACCC |
| GGGTCGGGGAGGGGCCAGAAGGGG | CCCCTTCTGGCCCCTCCCCGACCC |
| GGGTGATGAGGGGTGGGATGTGGG | CCCACATCCCACCCCTCATCACCC |
| GGGTGCGAGGGTGTGGGGAGTGGG | CCCACTCCCCACACCCTCGCACCC |
| GGGTGCTAGGGCGGGGCGGCTGGG | CCCAGCCGCCCCGCCCTAGCACCC |
| GGGTGGGAACTGGGGACCCAGGGG | CCCCTGGGTCCCCAGTTCCCACCC |
| GGGTGGGGCTGGGGTAGAGTTGGG | CCCAACTCTACCCCAGCCCCACCC |
| GGGTGGGGTAGAGAGGGTGAAGGG | CCCTTCACCCTCTCTACCCCACCC |
| GGGTGGTGGGGAGGAGGGGCTGGG | CCCAGCCCCTCCTCCCCACCACCC |
| GGGTGTGCGGGAGGAGGGGCGGGG | CCCCGCCCCTCCTCCCGCACACCC |
| GGGTTAATAGGGGCGGGGAGAGGG | CCCTCTCCCCGCCCCTATTAACCC |
| GGGTTAGGGCAGGGGCCGCGCGGG | CCCGCGCGGCCCCTGCCCTAACCC |
| GGGTTCTGGGTGGGGAGTGAGGGG | CCCCTCACTCCCCACCCAGAACCC |
| GGGTTGGGGGCGGGGCTGGGCGGG | CCCGCCCAGCCCCGCCCCCAACCC |
| CCCAAGCCCCTGACCCACGCTACCC | GGGTAGCGTGGGTCAGGGGCTTGGG |
| CCCACCCCCGTTTCCACCCATGCCC | GGGCATGGGTGGAAACGGGGGTGGG |
| CCCACCCCTTCCCTGCCCCTGCCCC | GGGGCAGGGGCAGGGAAGGGGTGGG |
| CCCACCTCCCTACTCCCAGCCCCCC | GGGGGGCTGGGAGTAGGGAGGTGGG |
| CCCACGCACCCACCCGCCCCTACCC | GGGTAGGGGCGGGTGGGTGCGTGGG |
| CCCACGGCCACCCACCCTTCTCCCC | GGGGAGAAGGGTGGGTGGCCGTGGG |
| CCCAGCCACACCCCACCCCCACCCC | GGGGTGGGGGTGGGGTGTGGCTGGG |
| CCCAGCCCTGCTCCCCCTCTCCCCC | GGGGGAGAGGGGGAGCAGGGCTGGG |
| CCCAGGACCCCTTCCCCCTGTACCC | GGGTACAGGGGGAAGGGGTCCTGGG |
| CCCAGGCCACCCGCCACCCCCTCCC | GGGAGGGGGTGGCGGGTGGCCTGGG |
| CCCAGGCCCCAATCCCGCAACGCCC | GGGCGTTGCGGGATTGGGGCCTGGG |
| CCCAGGCCCCACCCCCTCGACCCCC | GGGGGTCGAGGGGGTGGGGCCTGGG |
| CCCAGGCCCCGCCCCTGCCCCTCCC | GGGAGGGGCAGGGGCGGGGCCTGGG |
| CCCATGCCTCCCTCCTCCCCACCCC | GGGGTGGGGAGGAGGGAGGCATGGG |
| CCCCAACCCTCCCACCCAAAAACCC | GGGTTTTTGGGTGGGAGGGTTGGGG |
| CCCCAACCCTGGCCACCCCCTTCCC | GGGAAGGGGGTGGCCAGGGTTGGGG |
| CCCCACCCCAGCCCACCCCACCCCC | GGGGGTGGGGTGGGCTGGGGTGGGG |
| CCCCACCCCCATCCCACCCACCCCC | GGGGGTGGGTGGGATGGGGGTGGGG |
| CCCCACTCCCTGGCCCTCCCAGCCC | GGGCTGGGAGGGCCAGGGAGTGGGG |
| CCCCAGAATCCCAGCCCCACTTCCC | GGGAAGTGGGGCTGGGATTCTGGGG |
| CCCCAGCCCACACCCAGCACATCCC | GGGATGTGCTGGGTGTGGGCTGGGG |
| CCCCAGGCCCCCGCCCGCCCTGCCC | GGGCAGGGCGGGCGGGGGCCTGGGG |
| CCCCAGGTGCCCACCCAGGCTGCCC | GGGCAGCCTGGGTGGGCACCTGGGG |
| CCCCATGCCCCTGCTCCCTCAGCCC | GGGCTGAGGGAGCAGGGGCATGGGG |
| CCCCCAAGCCCTCTCAGCCCAACCC | GGGTTGGGCTGAGAGGGCTTGGGGG |
| CCCCCACCCACTATCCCATTTGCCC | GGGCAAATGGGATAGTGGGTGGGGG |
| CCCCCACCCCCAGGCCCTCTCTCCC | GGGAGAGAGGGCCTGGGGGTGGGGG |
| CCCCCACCCCGCCCGCCCCCTCCCC | GGGGAGGGGGCGGGCGGGGTGGGGG |
| CCCCCATCCCTTCCCCTTATCCCCC | GGGGGATAAGGGGAAGGGATGGGGG |
| CCCCCCACAACCCTCCCCGACACCC | GGGTGTCGGGGAGGGTTGTGGGGGG |
| CCCCCCACCCTTTCCCCATCATCCC | GGGATGATGGGGAAAGGGTGGGGGG |
| CCCCCCCAAAACCCCCCAACCCCCC | GGGGGGTTGGGGGGTTTTGGGGGGG |
| CCCCCCCACGGGCCCCGCCCATCCC | GGGATGGGCGGGGCCCGTGGGGGGG |
| CCCCCCCATCCCTCCCTCCATCCCC | GGGGATGGAGGGAGGGATGGGGGGG |
| CCCCCCCGCCCTGCCCGCGCCCCCC | GGGGGGCGCGGGCAGGGCGGGGGGG |
| CCCCCCGCCCCGCCCCCATCACCCC | GGGGTGATGGGGGCGGGGCGGGGGG |
| CCCCCCTCCCCCGCTACCCCTCCCC | GGGGAGGGGTAGCGGGGGAGGGGGG |
| CCCCCGCCCGCCGCCCCTCCCACCC | GGGTGGGAGGGGCGGCGGGCGGGGG |
| CCCCCGCCCGGCCCCACCCAGGCCC | GGGCCTGGGTGGGGCCGGGCGGGGG |
| CCCCCGCTTCCCCGTCCCTCCCCCC | GGGGGGAGGGACGGGGAAGCGGGGG |
| CCCCCTCCCCCGTGCGCCCCGCCCC | GGGGCGGGGCGCACGGGGGAGGGGG |
| CCCCCTCGCCCGCCCCGGCTCCCCC | GGGGGAGCCGGGGCGGGCGAGGGGG |
| CCCCGCCCAGTTCCCGCCCTCTCCC | GGGAGAGGGCGGGAACTGGGCGGGG |
| CCCCGCCCCCTGGCTCCCCGCCCCC | GGGGGCGGGGAGCCAGGGGGCGGGG |
| CCCCGCCCCGCACCCGCCGCCGCCC | GGGCGGCGGCGGGTGCGGGGCGGGG |
| CCCCGCCCCGCCCCCCACGTGCCCC | GGGGCACGTGGGGGGCGGGGCGGGG |
| CCCCGCCCCGTTACCCCTTGCCCCC | GGGGGCAAGGGGTAACGGGGCGGGG |
| CCCCGCCCCTACGACCCTCACCCCC | GGGGGTGAGGGTCGTAGGGGCGGGG |
| CCCCGCCCCTGCGGCCCCGGGTCCC | GGGACCCGGGGCCGCAGGGGCGGGG |
| CCCCGCCCTCCGCCCGCTCCCGCCC | GGGCGGGAGCGGGCGGAGGGCGGGG |
| CCCCGGATCCCACCCCTAACCCCCC | GGGGGGTTAGGGGTGGGATCCGGGG |
| CCCCGGTCCCTCCCCTCCCCACCCC | GGGGTGGGGAGGGGAGGGACCGGGG |
| CCCCTACCCCAGTCCCGTGTCGCCC | GGGCGACACGGGACTGGGGTAGGGG |
| CCCCTATGATCCCACCCACTGGCCC | GGGCCAGTGGGTGGGATCATAGGGG |
| CCCCTCCCAGCAGCTCCCCCTCCCC | GGGGAGGGGGAGCTGCTGGGAGGGG |
| CCCCTCCCCCAGACCCCAGGAGCCC | GGGCTCCTGGGGTCTGGGGGAGGGG |
| CCCCTCCCCCCCCACCCCAACCCCC | GGGGGTTGGGGTGGGGGGGGAGGGG |
| CCCCTCCCCGCTCCCGCCCAGGCCC | GGGCCTGGGCGGGAGCGGGGAGGGG |
| CCCCTCCCCTCAACAGCCCAGACCC | GGGTCTGGGCTGTTGACGGGAGGGG |
| CCCCTCGCCCTCCCCACTCCCACCC | GGGTGGGAGTGGGGAGGGCGAGGGG |
| CCCCTGCCCAGCCCCCTCGCCGCCC | GGGCGGCGAGGGGGCTGGGCAGGGG |
| CCCCTGGCCCAGATCCCTCTCCCCC | GGGGGAGAGGGATCTGGGCCAGGGG |
| CCCCTTCCCAAAGCCCTTCCCTCCC | GGGAGGGAAGGGCTTTGGGAAGGGG |
| CCCGACAATCCCTTATCCCTACCCC | GGGGTAGGGATAAGGGATTGTCGGG |
| CCCGAGCCCCAAGAGTCCCCTGCCC | GGGCAGGGGACTCTTGGGGCTCGGG |
| CCCGAGTACCCTCCCCCTTAGCCCC | GGGGCTAAGGGGGAGGGTACTCGGG |
| CCCGCACCCCACCCCACCGCACCCC | GGGGTGCGGTGGGGTGGGGTGCGGG |
| CCCGCCCCCGGCCCGGCCCGCCCCC | GGGGGCGGGCCGGGCCGGGGGCGGG |
| CCCGCCCCGCAGCCCTAGCAGCCCC | GGGGCTGCTAGGGCTGCGGGGCGGG |
| CCCGCCCCTCCTTTCCCAGCCACCC | GGGTGGCTGGGAAAGGAGGGGCGGG |
| CCCGCCCGCGCCCAGCCCCGCCCCC | GGGGGCGGGGCTGGGCGCGGGCGGG |
| CCCGCCCTGGGCCCCCCCTCACCCC | GGGGTGAGGGGGGGCCCAGGGCGGG |
| CCCGCGTCCCGCCCCCTCCTCCCCC | GGGGGAGGAGGGGGCGGGACGCGGG |
| CCCGCTCCCAGTCCCGGCCCCTCCC | GGGAGGGGCCGGGACTGGGAGCGGG |
| CCCGGCCCGCGGCCCCGCTAACCCC | GGGGTTAGCGGGGCCGCGGGCCGGG |
| CCCTCACGCCCCCAGGCCCCGCCCC | GGGGCGGGGCCTGGGGGCGTGAGGG |
| CCCTCCCCCTGTACCCAGGCCTCCC | GGGAGGCCTGGGTACAGGGGGAGGG |
| CCCTCCCCTGCTCCCAGCCCCCCCC | GGGGGGGGCTGGGAGCAGGGGAGGG |
| CCCTCCCGCCCCCCTCCCCGGGCCC | GGGCCCGGGGAGGGGGGCGGGAGGG |
| CCCTCCCGCCCCGGAGGCCCGGCCC | GGGCCGGGCCTCCGGGGCGGGAGGG |
| CCCTCCCTTGGACACCCCCACCCCC | GGGGGTGGGGGTGTCCAAGGGAGGG |
| CCCTCCGCCCCCCTTCCCGACACCC | GGGTGTCGGGAAGGGGGGCGGAGGG |
| CCCTCCGCCCCGCACCCCTAGGCCC | GGGCCTAGGGGTGCGGGGCGGAGGG |
| CCCTCCGCCCGCCGGCCCGCCCCCC | GGGGGGCGGGCCGGCGGGCGGAGGG |
| CCCTCGCCCTCACCCTTGCCCGCCC | GGGCGGGCAAGGGTGAGGGCGAGGG |
| CCCTCTACCCCCCCACCCCCACCCC | GGGGTGGGGGTGGGGGGGTAGAGGG |
| CCCTCTCCCTCCCTTCTTCCCACCC | GGGTGGGAAGAAGGGAGGGAGAGGG |
| CCCTCTCTCGCCCTCCATCCCCCCC | GGGGGGGATGGAGGGCGAGAGAGGG |
| CCCTCTGCCCCCCACCCCACACCCC | GGGGTGTGGGGTGGGGGGCAGAGGG |
| CCCTCTTCCCTCCTCCTCCCATCCC | GGGATGGGAGGAGGAGGGAAGAGGG |
| CCCTGAGAGTCCCCTCCCTTCTCCC | GGGAGAAGGGAGGGGACTCTCAGGG |
| CCCTGCCCACCCCGCCCCCAACCCC | GGGGTTGGGGGCGGGGTGGGCAGGG |
| CCCTGCCCCGGACCCAGCCCTGCCC | GGGCAGGGCTGGGTCCGGGGCAGGG |
| CCCTGCCTCCCTGTCCCCTCCACCC | GGGTGGAGGGGACAGGGAGGCAGGG |
| CCCTGCGGGCCCCGCCCTCAGTCCC | GGGACTGAGGGCGGGGCCCGCAGGG |
| CCCTGCTCAGCCCCCTGGCCCGCCC | GGGCGGGCCAGGGGGCTGAGCAGGG |
| CCCTGCTCCCATCTCCCCTTCCCCC | GGGGGAAGGGGAGATGGGAGCAGGG |
| CCCTGCTGCCCCACACGCCCCACCC | GGGTGGGGCGTGTGGGGCAGCAGGG |
| CCCTGCTGCCCTTGTGCCCCACCCC | GGGGTGGGGCACAAGGGCAGCAGGG |
| CCCTTCCCAATCCCTCCCTCACCCC | GGGGTGAGGGAGGGATTGGGAAGGG |
| CCCTTCCCAGTCTTTCCCGGCCCCC | GGGGGCCGGGAAAGACTGGGAAGGG |
| CCCTTCCCCACCAGCCCTTATCCCC | GGGGATAAGGGCTGGTGGGGAAGGG |
| CCCTTCCCCTTCCCGCCCGCTCCCC | GGGGAGCGGGCGGGAAGGGGAAGGG |
| CCCTTTTTTCCCCTTCTCCCTTCCC | GGGAAGGGAGAAGGGGAAAAAAGGG |
| GGGAAACAGGGAGGGGAGGAAAGGG | CCCTTTCCTCCCCTCCCTGTTTCCC |
| GGGAAAGGGGGAGCAGGGTACTGGG | CCCAGTACCCTGCTCCCCCTTTCCC |
| GGGAACAGGGAGAGGGCCCTGGGGG | CCCCCAGGGCCCTCTCCCTGTTCCC |
| GGGAAGGGTCATACAGGGGTCGGGG | CCCCGACCCCTGTATGACCCTTCCC |
| GGGACAGGGTCCGGGGTAGTCTGGG | CCCAGACTACCCCGGACCCTGTCCC |
| GGGACCTGGGGGAAGGGAAGCTGGG | CCCAGCTTCCCTTCCCCCAGGTCCC |
| GGGAGAGGGGGGCGGGTCATCTGGG | CCCAGATGACCCGCCCCCCTCTCCC |
| GGGAGAGGGGTTGGGAGACATGGGG | CCCCATGTCTCCCAACCCCTCTCCC |
| GGGAGGCCAGGGCCCGGGCTGGGGG | CCCCCAGCCCGGGCCCTGGCCTCCC |
| GGGAGGGAAGGAGAGGGAGGGTGGG | CCCACCCTCCCTCTCCTTCCCTCCC |
| GGGAGGGCTCTGCAGGGAAGAGGGG | CCCCTCTTCCCTGCAGAGCCCTCCC |
| GGGAGGGGGAGCGGGCAGCCCCGGG | CCCGGGGCTGCCCGCTCCCCCTCCC |
| GGGAGGGTGCGGCGGGCAGCGGGGG | CCCCCGCTGCCCGCCGCACCCTCCC |
| GGGAGGTGTGGGAATACTGGGGGGG | CCCCCCCAGTATTCCCACACCTCCC |
| GGGAGTGGGGGAATGGGGATGAGGG | CCCTCATCCCCATTCCCCCACTCCC |
| GGGAGTGGGGGAGGGCGCGGGCGGG | CCCGCCCGCGCCCTCCCCCACTCCC |
| GGGATGAGGTGGGGAAAGGGGTGGG | CCCACCCCTTTCCCCACCTCATCCC |
| GGGCAACCTGGGCAAGGGGCGTGGG | CCCACGCCCCTTGCCCAGGTTGCCC |
| GGGCAGCCTGGGTGGGCACCTGGGG | CCCCAGGTGCCCACCCAGGCTGCCC |
| GGGCCAGGGCCTCACTGGGGCAGGG | CCCTGCCCCAGTGAGGCCCTGGCCC |
| GGGCCCGGGCGGGAGAGAGGGAGGG | CCCTCCCTCTCTCCCGCCCGGGCCC |
| GGGCCCGGGCGGGGGCGGGGCGGGG | CCCCGCCCCGCCCCCGCCCGGGCCC |
| GGGCCCTGGGGCTCCTGGGCGCGGG | CCCGCGCCCAGGAGCCCCAGGGCCC |
| GGGCCGGGAGGTGGGAGAGGGAGGG | CCCTCCCTCTCCCACCTCCCGGCCC |
| GGGCCGGGCCGGGCCGGGACCGGGG | CCCCGGTCCCGGCCCGGCCCGGCCC |
| GGGCCGGGGCTGGGGCATTTCAGGG | CCCTGAAATGCCCCAGCCCCGGCCC |
| GGGCCGGTCGGGGAGCGGGCAGGGG | CCCCTGCCCGCTCCCCGACCGGCCC |
| GGGCGCAGGCGGGCTGGCGGGCGGG | CCCGCCCGCCAGCCCGCCTGCGCCC |
| GGGCGCGGGGTGGGCGCGGGGCGGG | CCCGCCCCGCGCCCACCCCGCGCCC |
| GGGCGGCGGGGGAGGGAACCGGGGG | CCCCCGGTTCCCTCCCCCGCCGCCC |
| GGGCGGGAGGGGGAGGGGCGCGGGG | CCCCGCGCCCCTCCCCCTCCCGCCC |
| GGGCGGGCGACAGGGCTCTCGGGGG | CCCCCGAGAGCCCTGTCGCCCGCCC |
| GGGCGGGGCCGAGCCGGGCCGTGGG | CCCACGGCCCGGCTCGGCCCCGCCC |
| GGGCGGGGTGCGGGGCATGGTGGGG | CCCCACCATGCCCCGCACCCCGCCC |
| GGGCGGGTCTCCCTGGGCGCGGGGG | CCCCCGCGCCCAGGGAGACCCGCCC |
| GGGCGTGGAGGGCGTGGGCAGGGGG | CCCCCTGCCCACGCCCTCCACGCCC |
| GGGCTCCCGGGGGCGGAGGGAAGGG | CCCTTCCCTCCGCCCCCGGGAGCCC |
| GGGCTGGGGAGGGGCGCGGGGAGGG | CCCTCCCCGCGCCCCTCCCCAGCCC |
| GGGCTGGGGCGGGGCTGGGGCGGGG | CCCCGCCCCAGCCCCGCCCCAGCCC |
| GGGCTGGGGGACCCGGGACCGGGGG | CCCCCGGTCCCGGGTCCCCCAGCCC |
| GGGCTTGGGCGGGATGGGGCAAGGG | CCCTTGCCCCATCCCGCCCAAGCCC |
| GGGGAAGGAAGGGAGGGAACAAGGG | CCCTTGTTCCCTCCCTTCCTTCCCC |
| GGGGAGCACTGGGTGGGGCTGTGGG | CCCACAGCCCCACCCAGTGCTCCCC |
| GGGGAGGCGGGGCACGGGGACTGGG | CCCAGTCCCCGTGCCCCGCCTCCCC |
| GGGGAGGCGGGTGGGGGAGGGAGGG | CCCTCCCTCCCCCACCCGCCTCCCC |
| GGGGAGGGAGGGCAGGGAAAGGGGG | CCCCCTTTCCCTGCCCTCCCTCCCC |
| GGGGAGGGAGGGGCGTGGGCCTGGG | CCCAGGCCCACGCCCCTCCCTCCCC |
| GGGGAGGGCCGGGCCGGGGCTAGGG | CCCTAGCCCCGGCCCGGCCCTCCCC |
| GGGGAGGGGACCCCGGGTGAGGGGG | CCCCCTCACCCGGGGTCCCCTCCCC |
| GGGGAGGGGACTGGAGGGGGGCGGG | CCCGCCCCCCTCCAGTCCCCTCCCC |
| GGGGAGGGGAGATGGGCGTGGAGGG | CCCTCCACGCCCATCTCCCCTCCCC |
| GGGGAGGGGGGAGGGACCGGATGGG | CCCATCCGGTCCCTCCCCCCTCCCC |
| GGGGAGGGGGTGGAGGGAGGGCGGG | CCCGCCCTCCCTCCACCCCCTCCCC |
| GGGGATGGGGCGTTTCTGGGAGGGG | CCCCTCCCAGAAACGCCCCATCCCC |
| GGGGCAAGGCGGGGCCGGGCGGGGG | CCCCCGCCCGGCCCCGCCTTGCCCC |
| GGGGCAGCCTGGGGAGGGACGCGGG | CCCGCGTCCCTCCCCAGGCTGCCCC |
| GGGGCAGGGGGAGGGGGGAAGGGGG | CCCCCTTCCCCCCTCCCCCTGCCCC |
| GGGGCCAGGGAGGGTGGGCACAGGG | CCCTGTGCCCACCCTCCCTGGCCCC |
| GGGGCCAGGGCGGGGTGGCCCAGGG | CCCTGGGCCACCCCGCCCTGGCCCC |
| GGGGCCAGGGGAGGGAGGGTCTGGG | CCCAGACCCTCCCTCCCCTGGCCCC |
| GGGGCCGGGGGTGGGCATCACTGGG | CCCAGTGATGCCCACCCCCGGCCCC |
| GGGGCTGGCGGGAGGGCTGGGTGGG | CCCACCCAGCCCTCCCGCCAGCCCC |
| GGGGCTGGGGGGCCGGGGTGGGGGG | CCCCCCACCCCGGCCCCCCAGCCCC |
| GGGGCTGGGGGTATCGGAGGGGGGG | CCCCCCCTCCGATACCCCCAGCCCC |
| GGGGCTGGGTGCGGGGCGGGGCGGG | CCCGCCCCGCCCCGCACCCAGCCCC |
| GGGGCTTGGGGATGGGTTTGGAGGG | CCCTCCAAACCCATCCCCAAGCCCC |
| GGGGGAAGTGGGAGCTGGGTGGGGG | CCCCCACCCAGCTCCCACTTCCCCC |
| GGGGGAGGGAAAGGAGGGGGGCGGG | CCCGCCCCCCTCCTTTCCCTCCCCC |
| GGGGGAGGGGAAGGGGGCGATGGGG | CCCCATCGCCCCCTTCCCCTCCCCC |
| GGGGGAGGGGGAGCGGGCCTTCGGG | CCCCGAAGGCCCGTCCCCCTCCCCC |
| GGGGGAGGGGGCCCGGAGGGAAGGG | CCCTTCCCTCCGGGCCCCCTCCCCC |
| GGGGGAGGGGTGGCTGGGATTTGGG | CCCAAATCCCAGCCACCCCTCCCCC |
| GGGGGAGTGGGGGCTGGGGCAGGGG | CCCCTGCCCCAGCCCCCACTCCCCC |
| GGGGGCCCAAGGGAGGGGGCCTGGG | CCCAGGCCCCCTCCCTTGGGCCCCC |
| GGGGGCCGGGCTGGGCTGCGCCGGG | CCCGGCGCAGCCCAGCCCGGCCCCC |
| GGGGGCCGGGGCCGGGACCGCGGGG | CCCCGCGGTCCCGGCCCCGGCCCCC |
| GGGGGCCGGGGCGCGGGCTCAGGGG | CCCCTGAGCCCGCGCCCCGGCCCCC |
| GGGGGCGCCGGGCACGCGGGCTGGG | CCCAGCCCGCGTGCCCGGCGCCCCC |
| GGGGGCGGGGTCCGGACGGGCGGGG | CCCCGCCCCTCCGGACCCCGCCCCC |
| GGGGGGAAGGGAGGCGGGGGAAGGG | CCCTTCCCCCGCCTCCCTTCCCCCC |
| GGGGGGCGGGCTGGGGCTGGTGGGG | CCCCACCAGCCCCAGCCCGCCCCCC |
| GGGGGGGCCAGGATGGGGGGAGGGG | CCCCTCCCCCCATCCTGGCCCCCCC |
| GGGGGGTGGGGCTGGAGGGCCTGGG | CCCAGGCCCTCCAGCCCCACCCCCC |
| GGGGGTATGGGCCTGGGGACCTGGG | CCCAGGTCCCCAGGCCCATACCCCC |
| GGGGTAAGGGTTTGGGAAGTCAGGG | CCCTGACTTCCCAAACCCTTACCCC |
| GGGGTACGGGGGCCGGGATGGAGGG | CCCTCCATCCCGGCCCCCGTACCCC |
| GGGGTAGGGATGAGGGAGGGAGGGG | CCCCTCCCTCCCTCATCCCTACCCC |
| GGGGTGGGGGCAGGGGTAGGGCGGG | CCCGCCCTACCCCTGCCCCCACCCC |
| GGGGTGGGGGGTGGGGATGGGAGGG | CCCTCCCATCCCCACCCCCCACCCC |
| GGGTAGACCTGGGGTGGGGGTAGGG | CCCTACCCCCACCCCAGGTCTACCC |
| GGGTCCGGGGCCCCCTGGGCGGGGG | CCCCCGCCCAGGGGGCCCCGGACCC |
| GGGTCTCAGGGCCGGGCAGTCTGGG | CCCAGACTGCCCGGCCCTGAGACCC |
| GGGTCTGGGCTGTTAAGGGGAGGGG | CCCCTCCCCTTAACAGCCCAGACCC |
| GGGTGAGAGGGGGAGTGGGCTGGGG | CCCCAGCCCACTCCCCCTCTCACCC |
| GGGTGCAGGGACTGAGGGGGAGGGG | CCCCTCCCCCTCAGTCCCTGCACCC |
| GGGTGGATGGGGAAGGGACAGGGGG | CCCCCTGTCCCTTCCCCATCCACCC |
| GGGTGGCCCGGGGAGGGGAAAAGGG | CCCTTTTCCCCTCCCCGGGCCACCC |
| GGGTGGCTGGGGGCGGGGCTGGGGG | CCCCCAGCCCCGCCCCCAGCCACCC |
| GGGTGGGAGCGATGGGTAGGAGGGG | CCCCTCCTACCCATCGCTCCCACCC |
| GGGTGGGGGCCTGGGCTGGGGTGGG | CCCACCCCAGCCCAGGCCCCCACCC |
| GGGTGGTGGGGGAGGGGGTGGGGGG | CCCCCCACCCCCTCCCCCACCACCC |
| GGGTGTGGCCGGGGCGGGGCCGGGG | CCCCGGCCCCGCCCCGGCCACACCC |
| GGGTTGGGGGCGGGGGCGGGGGGGG | CCCCCCCCGCCCCCGCCCCCAACCC |
| CCCAACACCCTCCACCCCAGTCCCCC | GGGGGACTGGGGTGGAGGGTGTTGGG |
| CCCACAGCCCCCGCCCCTTCAGCCCC | GGGGCTGAAGGGGCGGGGGCTGTGGG |
| CCCACCCTCCCTCCCCCATCCTCCCC | GGGGAGGATGGGGGAGGGAGGGTGGG |
| CCCACCTGCCCTTGCCCCCAGGACCC | GGGTCCTGCGGGCAAGGGCAGGTGGG |
| CCCAGCCCAGCCCCCACCCCTTCCCC | GGGGAAGGGGTGGGGGCTGGGCTGGG |
| CCCAGCCTGCCCCGTCTGGCCCACCC | GGGTGGGCCAGACGGGGCAGGCTGGG |
| CCCAGGCCAGCCCCCGCCCCAGCCCC | GGGGCTGGGGCGGGGGCTGGCCTGGG |
| CCCAGGCCCCACCCCCGGCCCGCCCC | GGGGCGGGCCGGGGGTGGGGCCTGGG |
| CCCATTTTCCCAGGACCCCTCCTCCC | GGGAGGAGGGGTCCTGGGAAAATGGG |
| CCCCAACCCCACCTCCCTTCCCGCCC | GGGCGGGAAGGGAGGTGGGGTTGGGG |
| CCCCAACCCTGCCTCCCTCCCCACCC | GGGTGGGGAGGGAGGCAGGGTTGGGG |
| CCCCAACCCTTCCTCCCTCCCCACCC | GGGTGGGGAGGGAGGAAGGGTTGGGG |
| CCCCAAGCCCGCCTCCCTCCCCACCC | GGGTGGGGAGGGAGGCGGGCTTGGGG |
| CCCCACCCAGAAAGCCCCGGGCGCCC | GGGCGCCCGGGGCTTTCTGGGTGGGG |
| CCCCACCCCCCTCCCCCAATGACCCC | GGGGTCATTGGGGGAGGGGGGTGGGG |
| CCCCACCCCCTGCTCCCCGAGAGCCC | GGGCTCTCGGGGAGCAGGGGGTGGGG |
| CCCCAGAGGCCCCTCTCCCCAGACCC | GGGTCTGGGGAGAGGGGCCTCTGGGG |
| CCCCAGATCCCTGCCCTGGAGAGCCC | GGGCTCTCCAGGGCAGGGATCTGGGG |
| CCCCAGCCCCTGGCCCTCCCGGTCCC | GGGACCGGGAGGGCCAGGGGCTGGGG |
| CCCCAGCCCTTCTCCCCTCCACGCCC | GGGCGTGGAGGGGAGAAGGGCTGGGG |
| CCCCAGGCCCAGCCCCAGCCCTGCCC | GGGCAGGGCTGGGGCTGGGCCTGGGG |
| CCCCAGGGCCCCGCCCACGCCGACCC | GGGTCGGCGTGGGCGGGGCCCTGGGG |
| CCCCAGTACCCCGGCCCCGACCCCCC | GGGGGGTCGGGGCCGGGGTACTGGGG |
| CCCCATCCCGCGGTGCCCTGTCGCCC | GGGCGACAGGGCACCGCGGGATGGGG |
| CCCCCAACCTCCCTACCCCCTCCCCC | GGGGGAGGGGGTAGGGAGGTTGGGGG |
| CCCCCACCCCAGCCCCGGCCCGCCCC | GGGGCGGGCCGGGGCTGGGGTGGGGG |
| CCCCCACTCCCAGCCCCTCCCCCCCC | GGGGGGGGAGGGGCTGGGAGTGGGGG |
| CCCCCAGCCCAGCCCCGACCCTGCCC | GGGCAGGGTCGGGGCTGGGCTGGGGG |
| CCCCCAGCCCCAGCCCCTGCCATCCC | GGGATGGCAGGGGCTGGGGCTGGGGG |
| CCCCCAGCCCCCGTCCCGCGGCCCCC | GGGGGCCGCGGGACGGGGGCTGGGGG |
| CCCCCCAGCCCCAGCCCGAGACCCCC | GGGGGTCTCGGGCTGGGGCTGGGGGG |
| CCCCCCCACCCAGGCCCCCGCCACCC | GGGTGGCGGGGGCCTGGGTGGGGGGG |
| CCCCCCCCCCCCGCCCCTTCCACCCC | GGGGTGGAAGGGGCGGGGGGGGGGGG |
| CCCCCCGCCCCGCGGCCCCGGGCCCC | GGGGCCCGGGGCCGCGGGGCGGGGGG |
| CCCCCCTCCCCCTCCCCAGAACCCCC | GGGGGTTCTGGGGAGGGGGAGGGGGG |
| CCCCCCTCCCGGCCCCGTTTCAGCCC | GGGCTGAAACGGGGCCGGGAGGGGGG |
| CCCCCGCCCCCGAGGCCCGCTTCCCC | GGGGAAGCGGGCCTCGGGGGCGGGGG |
| CCCCCGGCCCCCGCCTCCGCCCTCCC | GGGAGGGCGGAGGCGGGGGCCGGGGG |
| CCCCCTAACCCGCCAGCCCCGCCCCC | GGGGGCGGGGCTGGCGGGTTAGGGGG |
| CCCCCTCCCTCTTCCCATCCCCTCCC | GGGAGGGGATGGGAAGAGGGAGGGGG |
| CCCCCTCCCTGTCCCCTAACCCTCCC | GGGAGGGTTAGGGGACAGGGAGGGGG |
| CCCCCTGCCCCCTCCCCCACCTTCCC | GGGAAGGTGGGGGAGGGGGCAGGGGG |
| CCCCGACCCCGCCCGCCCGCCTGCCC | GGGCAGGCGGGCGGGCGGGGTCGGGG |
| CCCCGCCCCCTCACCCTGGGGCTCCC | GGGAGCCCCAGGGTGAGGGGGCGGGG |
| CCCCGCCCCCTCCCCGCCCGCTGCCC | GGGCAGCGGGCGGGGAGGGGGCGGGG |
| CCCCGCCCCGACTGCCCGCGCCTCCC | GGGAGGCGCGGGCAGTCGGGGCGGGG |
| CCCCGCGGCTCCCTCCCTCCCTCCCC | GGGGAGGGAGGGAGGGAGCCGCGGGG |
| CCCCGCTCCCGGCCCCGCCGCCGCCC | GGGCGGCGGCGGGGCCGGGAGCGGGG |
| CCCCGGCCCAGCCCCCTGGGGACCCC | GGGGTCCCCAGGGGGCTGGGCCGGGG |
| CCCCGGCCCGGAGCCGCCCGGGCCCC | GGGGCCCGGGCGGCTCCGGGCCGGGG |
| CCCCGGCTACCCCGCCCAGAGCCCCC | GGGGGCTCTGGGCGGGGTAGCCGGGG |
| CCCCGTCCCCACCCCCAGTGCCACCC | GGGTGGCACTGGGGGTGGGGACGGGG |
| CCCCTACCCCCCCACCCCCTGCACCC | GGGTGCAGGGGGTGGGGGGGTAGGGG |
| CCCCTAGCGCCCCATCTCCCTGTCCC | GGGACAGGGAGATGGGGCGCTAGGGG |
| CCCCTATCCACCCCCACCCACGCCCC | GGGGCGTGGGTGGGGGTGGATAGGGG |
| CCCCTCCCACCCTTCCCAATCCGCCC | GGGCGGATTGGGAAGGGTGGGAGGGG |
| CCCCTCCCCTCAGCCCCCTCCACCCC | GGGGTGGAGGGGGCTGAGGGGAGGGG |
| CCCCTCCCGTCCCTGCCCCTCCTCCC | GGGAGGAGGGGCAGGGACGGGAGGGG |
| CCCCTCCTCCCGCACCCACCCATCCC | GGGATGGGTGGGTGCGGGAGGAGGGG |
| CCCCTCGCCCCCGCCCCCCTGCCCCC | GGGGGCAGGGGGGCGGGGGCGAGGGG |
| CCCCTCTGCCCCGGGACCCCTTCCCC | GGGGAAGGGGTCCCGGGGCAGAGGGG |
| CCCCTGCCCCACCCCCTTCCCGCCCC | GGGGCGGGAAGGGGGTGGGGCAGGGG |
| CCCCTGGAGACCCCTCCCCACTCCCC | GGGGAGTGGGGAGGGGTCTCCAGGGG |
| CCCCTTCCCCAGTGCCCCAGGGGCCC | GGGCCCCTGGGGCACTGGGGAAGGGG |
| CCCCTTCCCCTGACCCTTTTGCCCCC | GGGGGCAAAAGGGTCAGGGGAAGGGG |
| CCCCTTCCCGGCCCCCTGGCCTCCCC | GGGGAGGCCAGGGGGCCGGGAAGGGG |
| CCCCTTCCCTATCAAACCCCACCCCC | GGGGGTGGGGTTTGATAGGGAAGGGG |
| CCCGACCCCCCTCCCCTTCGCTTCCC | GGGAAGCGAAGGGGAGGGGGGTCGGG |
| CCCGAGCCCAGCGCCCGCGCCTGCCC | GGGCAGGCGCGGGCGCTGGGCTCGGG |
| CCCGATTGCCCCGGTCCCAGCAGCCC | GGGCTGCTGGGACCGGGGCAATCGGG |
| CCCGCCGGGCCCGCCCCCGGAGCCCC | GGGGCTCCGGGGGCGGGCCCGGCGGG |
| CCCGCCTCCTCCCGCCCCCTCGTCCC | GGGACGAGGGGGCGGGAGGAGGCGGG |
| CCCGCGCCCCGATTGGCCCACTTCCC | GGGAAGTGGGCCAATCGGGGCGCGGG |
| CCCGGCAGCCCTCCCCCAGCCCACCC | GGGTGGGCTGGGGGAGGGCTGCCGGG |
| CCCGGCCCCCGCCTGCCCTCCGTCCC | GGGACGGAGGGCAGGCGGGGGCCGGG |
| CCCGGCGCCCGACCCCGCCCGGCCCC | GGGGCCGGGCGGGGTCGGGCGCCGGG |
| CCCGTCCCTCCCTTCCCCTCCCGCCC | GGGCGGGAGGGGAAGGGAGGGACGGG |
| CCCGTCCGCCCGGCTTCCCCGCTCCC | GGGAGCGGGGAAGCCGGGCGGACGGG |
| CCCTAGCCCCTTCCCCGTCCCTTCCC | GGGAAGGGACGGGGAAGGGGCTAGGG |
| CCCTCCCACACCCCACCCCTAACCCC | GGGGTTAGGGGTGGGGTGTGGGAGGG |
| CCCTCCCACTTAACCCCACCTGGCCC | GGGCCAGGTGGGGTTAAGTGGGAGGG |
| CCCTCCCGACCCTCCCGAGTTCGCCC | GGGCGAACTCGGGAGGGTCGGGAGGG |
| CCCTCCCTCCACTCCCCTGTGGCCCC | GGGGCCACAGGGGAGTGGAGGGAGGG |
| CCCTCCCTTCCCTCTCCCTACACCCC | GGGGTGTAGGGAGAGGGAAGGGAGGG |
| CCCTGACCGCCCTGCCCGCATGCCCC | GGGGCATGCGGGCAGGGCGGTCAGGG |
| CCCTGCCCCTCCTCTCCCAGTCTCCC | GGGAGACTGGGAGAGGAGGGGCAGGG |
| CCCTGCTGTGCCCTCTGCCCACCCCC | GGGGGTGGGCAGAGGGCACAGCAGGG |
| CCCTGGCTCCCGGACACCCGACCCCC | GGGGGTCGGGTGTCCGGGAGCCAGGG |
| CCCTGGGCTCCCCCACCCAAGACCCC | GGGGTCTTGGGTGGGGGAGCCCAGGG |
| CCCTGTAGCCCCGCGGCCCCACACCC | GGGTGTGGGGCCGCGGGGCTACAGGG |
| CCCTGTCCCGCCTTGGCCCCGCCCCC | GGGGGCGGGGCCAAGGCGGGACAGGG |
| CCCTTCCCCCTCCCTCCCCAGGACCC | GGGTCCTGGGGAGGGAGGGGGAAGGG |
| CCCTTCCCCTCCCTTCTCCCATCCCC | GGGGATGGGAGAAGGGAGGGGAAGGG |
| CCCTTCCCTGTGTCCCCAATGCCCCC | GGGGGCATTGGGGACACAGGGAAGGG |
| CCCTTGACCCCCACCCACCCCCACCC | GGGTGGGGGTGGGTGGGGGTCAAGGG |
| CCCTTTAACCCCGCACCCCAAAGCCC | GGGCTTTGGGGTGCGGGGTTAAAGGG |
| GGGAAAAAGGGTGAAGGGGTTGTGGG | CCCACAACCCCTTCACCCTTTTTCCC |
| GGGAAACGCGGGAAGCAGGGGCGGGG | CCCCGCCCCTGCTTCCCGCGTTTCCC |
| GGGAATGGGAGGAAGTGGGAACAGGG | CCCTGTTCCCACTTCCTCCCATTCCC |
| GGGACAGGGGTGTCAGGGGGAGGGGG | CCCCCTCCCCCTGACACCCCTGTCCC |
| GGGAGGCTGGGGCGGGAGGTGCCGGG | CCCGGCACCTCCCGCCCCAGCCTCCC |
| GGGAGGGAGAAAGAGGGAGGGAAGGG | CCCTTCCCTCCCTCTTTCTCCCTCCC |
| GGGAGGGGAGGGGAGGGGTTGGGGGG | CCCCCCAACCCCTCCCCTCCCCTCCC |
| GGGAGGGGCCGGGGGGCATGATGGGG | CCCCATCATGCCCCCCGGCCCCTCCC |
| GGGAGGGGGATGGGGGTGAGGGTGGG | CCCACCCTCACCCCCATCCCCCTCCC |
| GGGAGGGGGGAGGGGGGTAGGCTGGG | CCCAGCCTACCCCCCTCCCCCCTCCC |
| GGGAGGGGGTGGGCGGGGGAGGAGGG | CCCTCCTCCCCCGCCCACCCCCTCCC |
| GGGAGGGTGGGCATGGGGCACTGGGG | CCCCAGTGCCCCATGCCCACCCTCCC |
| GGGATGGGGATGGGAGGGGGGGCGGG | CCCGCCCCCCCTCCCATCCCCATCCC |
| GGGCACTGGGGTGGGGACAGGGTGGG | CCCACCCTGTCCCCACCCCAGTGCCC |
| GGGCAGGAGGGGAGGGAGGCCAAGGG | CCCTTGGCCTCCCTCCCCTCCTGCCC |
| GGGCAGGGGAGCCTCGGGAATTTGGG | CCCAAATTCCCGAGGCTCCCCTGCCC |
| GGGCAGGGGGATGGGGAGGGAGTGGG | CCCACTCCCTCCCCATCCCCCTGCCC |
| GGGCAGGTGGGGTGGCGGGGGCGGGG | CCCCGCCCCCGCCACCCCACCTGCCC |
| GGGCCAGGGCTGGGGGTGGGGCGGGG | CCCCGCCCCACCCCCAGCCCTGGCCC |
| GGGCCGCGGGGCGGGGCCAGGGCGGG | CCCGCCCTGGCCCCGCCCCGCGGCCC |
| GGGCCGGGGGGAAGTGGGGGAGAGGG | CCCTCTCCCCCACTTCCCCCCGGCCC |
| GGGCGCGGGGCGGAGAGGGCGCGGGG | CCCCGCGCCCTCTCCGCCCCGCGCCC |
| GGGCGGGAAAGGGGGCGGCGGGGGGG | CCCCCCCGCCGCCCCCTTTCCCGCCC |
| GGGCGGGCGGGCGGGGCGCCGCTGGG | CCCAGCGGCGCCCCGCCCGCCCGCCC |
| GGGCGGGGCGGGGCGGGCCAAGAGGG | CCCTCTTGGCCCGCCCCGCCCCGCCC |
| GGGCGGGTGGGGAGGGCAGCCCGGGG | CCCCGGGCTGCCCTCCCCACCCGCCC |
| GGGCGGGTGGGGCGCGGTGGGCGGGG | CCCCGCCCACCGCGCCCCACCCGCCC |
| GGGCTCCGGGTGGCGCGGGCGGTGGG | CCCACCGCCCGCGCCACCCGGAGCCC |
| GGGCTGCGGGGCTGGGCGTCCCGGGG | CCCCGGGACGCCCAGCCCCGCAGCCC |
| GGGCTGCTCTGGGACGGGGCCGGGGG | CCCCCGGCCCCGTCCCAGAGCAGCCC |
| GGGCTGGGATGGGGCTGGGGTTGGGG | CCCCAACCCCAGCCCCATCCCAGCCC |
| GGGCTGGGGCAGGGCTGGGGGCGGGG | CCCCGCCCCCAGCCCTGCCCCAGCCC |
| GGGCTGGGTGGGAGGTGTGGGGTGGG | CCCACCCCACACCTCCCACCCAGCCC |
| GGGCTGTGGGGTGGAGGGGAGGGGGG | CCCCCCTCCCCTCCACCCCACAGCCC |
| GGGGAAGGGACCGCAGGGGGAGGGGG | CCCCCTCCCCCTGCGGTCCCTTCCCC |
| GGGGAAGGGGGCTGAGAGGGTCTGGG | CCCAGACCCTCTCAGCCCCCTTCCCC |
| GGGGACAGAGGGGAGCAAGGGAGGGG | CCCCTCCCTTGCTCCCCTCTGTCCCC |
| GGGGACATGGGATGGGGGAGGGAGGG | CCCTCCCTCCCCCATCCCATGTCCCC |
| GGGGACCCCGGGGACTGGGGTGGGGG | CCCCCACCCCAGTCCCCGGGGTCCCC |
| GGGGAGAGGGGGATGGGCTGCTGGGG | CCCCAGCAGCCCATCCCCCTCTCCCC |
| GGGGAGCCGTGGGCAAGGGGCGGGGG | CCCCCGCCCCTTGCCCACGGCTCCCC |
| GGGGAGGAGGGGAGGGGTGGAGGGGG | CCCCCTCCACCCCTCCCCTCCTCCCC |
| GGGGAGGGATTAGCAGGGGGAAGGGG | CCCCTTCCCCCTGCTAATCCCTCCCC |
| GGGGCACAAGGGAGGGCCAGGCTGGG | CCCAGCCTGGCCCTCCCTTGTGCCCC |
| GGGGCACCCTGGGCAGGGTGGGGGGG | CCCCCCCACCCTGCCCAGGGTGCCCC |
| GGGGCAGGGCTGGGGGTGGAGCCGGG | CCCGGCTCCACCCCCAGCCCTGCCCC |
| GGGGCATGGGGAGAAAGGGGCGTGGG | CCCACGCCCCTTTCTCCCCATGCCCC |
| GGGGCCGGAGGGCTGGGGAGAGTGGG | CCCACTCTCCCCAGCCCTCCGGCCCC |
| GGGGCCTGGGCAGGGGATGGGGAGGG | CCCTCCCCATCCCCTGCCCAGGCCCC |
| GGGGCGGGCCGGGCCGGGTTCCGGGG | CCCCGGAACCCGGCCCGGCCCGCCCC |
| GGGGCGGGGCCTCGGGTGCGGGCGGG | CCCGCCCGCACCCGAGGCCCCGCCCC |
| GGGGCGGGGGTCTGGGGGCCTGGGGG | CCCCCAGGCCCCCAGACCCCCGCCCC |
| GGGGCTATTGGGGACATGGGTGAGGG | CCCTCACCCATGTCCCCAATAGCCCC |
| GGGGCTCTGGGCCGGGCCGGCGCGGG | CCCGCGCCGGCCCGGCCCAGAGCCCC |
| GGGGCTGGAGGGCGGGGAGGCGGGGG | CCCCCGCCTCCCCGCCCTCCAGCCCC |
| GGGGCTGGGAGCAGATGGGGAGTGGG | CCCACTCCCCATCTGCTCCCAGCCCC |
| GGGGCTGGGTCAGGGGCTACTGTGGG | CCCACAGTAGCCCCTGACCCAGCCCC |
| GGGGGAAAGGGGTGGGCGGGGAGGGG | CCCCTCCCCGCCCACCCCTTTCCCCC |
| GGGGGACCGGGGCCGGGGCGCAGGGG | CCCCTGCGCCCCGGCCCCGGTCCCCC |
| GGGGGAGAGGGGAAGACGGGGAGGGG | CCCCTCCCCGTCTTCCCCTCTCCCCC |
| GGGGGAGGCGGGGGCCTGGGTGGGGG | CCCCCACCCAGGCCCCCGCCTCCCCC |
| GGGGGATGGGTATGGAAGGGTGGGGG | CCCCCACCCTTCCATACCCATCCCCC |
| GGGGGCAGGGGTTGCTGGGCCAGGGG | CCCCTGGCCCAGCAACCCCTGCCCCC |
| GGGGGCCCGGGCGGGGTCGCTAAGGG | CCCTTAGCGACCCCGCCCGGGCCCCC |
| GGGGGCCGGGGGCGAGGGCATGGGGG | CCCCCATGCCCTCGCCCCCGGCCCCC |
| GGGGGCGCGGGTGGGGCTGTTCGGGG | CCCCGAACAGCCCCACCCGCGCCCCC |
| GGGGGCGGAGGGGTAGGGGTGGGGGG | CCCCCCACCCCTACCCCTCCGCCCCC |
| GGGGGCGGGAGTAAGGGCCCTGGGGG | CCCCCAGGGCCCTTACTCCCGCCCCC |
| GGGGGCGGGGAGCCGGCGGGGGAGGG | CCCTCCCCCGCCGGCTCCCCGCCCCC |
| GGGGGCGGGGCAGAAGAGGGTGAGGG | CCCTCACCCTCTTCTGCCCCGCCCCC |
| GGGGGCGGGGCGGGGCGGGGGCGGGG | CCCCGCCCCCGCCCCGCCCCGCCCCC |
| GGGGGCGGGGCGGGGGGGCGGCGGGG | CCCCGCCGCCCCCCCGCCCCGCCCCC |
| GGGGGCGGGGCGGTGGGGGAGGGGGG | CCCCCCTCCCCCACCGCCCCGCCCCC |
| GGGGGCGGGGGCGGGGCGCCAAGGGG | CCCCTTGGCGCCCCGCCCCCGCCCCC |
| GGGGGCTGTGGGGGTGGGGTGAGGGG | CCCCTCACCCCACCCCCACAGCCCCC |
| GGGGGGAGGGGACGGGGGCAGAGGGG | CCCCTCTGCCCCCGTCCCCTCCCCCC |
| GGGGGTAGGGATGGGGCCTAGGTGGG | CCCACCTAGGCCCCATCCCTACCCCC |
| GGGGGTAGGGGCTGGATGGGAGTGGG | CCCACTCCCATCCAGCCCCTACCCCC |
| GGGGGTGGGCGGGCGGGGCGCGGGGG | CCCCCGCGCCCCGCCCGCCCACCCCC |
| GGGGGTGGGGGGCCGGGTGGGCCGGG | CCCGGCCCACCCGGCCCCCCACCCCC |
| GGGGGTGTGGGGTGCCTGGGATGGGG | CCCCATCCCAGGCACCCCACACCCCC |
| GGGGTATCGCGGGTAGGGGACTTGGG | CCCAAGTCCCCTACCCGCGATACCCC |
| GGGGTCCCGGGAGAGGGGTTCCGGGG | CCCCGGAACCCCTCTCCCGGGACCCC |
| GGGGTGAGAGGGGGGGCCGCGGAGGG | CCCTCCGCGGCCCCCCCTCTCACCCC |
| GGGGTGAGGCGGGCTGGGGTTTTGGG | CCCAAAACCCCAGCCCGCCTCACCCC |
| GGGGTGGGGCCGGTGGGGGAGGAGGG | CCCTCCTCCCCCACCGGCCCCACCCC |
| GGGGTTCGGGCTCGGGGGGCGGGGGG | CCCCCCGCCCCCCGAGCCCGAACCCC |
| GGGGTTTGGGAACATGAGGGGTGGGG | CCCCACCCCTCATGTTCCCAAACCCC |
| GGGTAAGGTGGGAAAGGGGTGTGGGG | CCCCACACCCCTTTCCCACCTTACCC |
| GGGTAATGGGTGTGGGAAGCTGTGGG | CCCACAGCTTCCCACACCCATTACCC |
| GGGTACGAGGGCGGCCGGGGGTGGGG | CCCCACCCCCGGCCGCCCTCGTACCC |
| GGGTGAAGGGGCTGGGCAGGTGGGGG | CCCCCACCTGCCCAGCCCCTTCACCC |
| GGGTGGGGAGGGAGGAAGGGTTGGGG | CCCCAACCCTTCCTCCCTCCCCACCC |
| GGGTGGGGAGGGAGGCAGGGTTGGGG | CCCCAACCCTGCCTCCCTCCCCACCC |
| GGGTGGGGAGGGAGGCGGGCTTGGGG | CCCCAAGCCCGCCTCCCTCCCCACCC |
| GGGTGTAGGGGTAGGTGGGCTTGGGG | CCCCAAGCCCACCTACCCCTACACCC |
| GGGTGTGGGCCAGGGGTGGGGCTGGG | CCCAGCCCCACCCCTGGCCCACACCC |
| GGGTTCAGAGGGCGGGGCGCGAGGGG | CCCCTCGCGCCCCGCCCTCTGAACCC |
| GGGTTGGTGGGTTAGGGGGCTGGGGG | CCCCCAGCCCCCTAACCCACCAACCC |
| CCCAAGGTGACCCTTGGCCCCCAACCC | GGGTTGGGGGCCAAGGGTCACCTTGGG |
| CCCAAGTCCCCGAGCCCACCCCAGCCC | GGGCTGGGGTGGGCTCGGGGACTTGGG |
| CCCAATCCCCCTACCCGCCCTCTGCCC | GGGCAGAGGGCGGGTAGGGGGATTGGG |
| CCCACATTGCCCAAACCCAAACTTCCC | GGGAAGTTTGGGTTTGGGCAATGTGGG |
| CCCACCGCGTCCCCGCCCCCAGGCCCC | GGGGCCTGGGGGCGGGGACGCGGTGGG |
| CCCACCTCCCCCACCCCTGTGCAACCC | GGGTTGCACAGGGGTGGGGGAGGTGGG |
| CCCACCTGGCCCCACCCTGCCTCACCC | GGGTGAGGCAGGGTGGGGCCAGGTGGG |
| CCCACGGCCCCCTCCCCAGTCCTCCCC | GGGGAGGACTGGGGAGGGGGCCGTGGG |
| CCCACGGGCGCCCCTGGGCCCTGTCCC | GGGACAGGGCCCAGGGGCGCCCGTGGG |
| CCCACTCCCCTCCTGGCCCACCCACCC | GGGTGGGTGGGCCAGGAGGGGAGTGGG |
| CCCAGCCCCTGGGCTCCCCCCAGCCCC | GGGGCTGGGGGGAGCCCAGGGGCTGGG |
| CCCAGCCCGCGGAACCCCGGCGGCCCC | GGGGCCGCCGGGGTTCCGCGGGCTGGG |
| CCCAGCCGGCCCCGCCCATACCTTCCC | GGGAAGGTATGGGCGGGGCCGGCTGGG |
| CCCAGCGACTCCCCCTCCCCCTCCCCC | GGGGGAGGGGGAGGGGGAGTCGCTGGG |
| CCCAGTTCTCCCTCCCCAACTCTTCCC | GGGAAGAGTTGGGGAGGGAGAACTGGG |
| CCCAGTTTTCCCTCCCCCTCTACCCCC | GGGGGTAGAGGGGGAGGGAAAACTGGG |
| CCCATGCCCGCCCGGCCCCTCCCGCCC | GGGCGGGAGGGGCCGGGCGGGCATGGG |
| CCCCAACCCCGCCTCCCTCCCCACCCC | GGGGTGGGGAGGGAGGCGGGGTTGGGG |
| CCCCAACCCCGCTTCCCTCCCCACCCC | GGGGTGGGGAGGGAAGCGGGGTTGGGG |
| CCCCAACCCTACCTCCCTCCCCACCCC | GGGGTGGGGAGGGAGGTAGGGTTGGGG |
| CCCCAAGAGGCCCCAACCCCTTTTCCC | GGGAAAAGGGGTTGGGGCCTCTTGGGG |
| CCCCACTCACCCACCTGCTCCCTTCCC | GGGAAGGGAGCAGGTGGGTGAGTGGGG |
| CCCCAGCCCCGCGGGGCCCCGGGTCCC | GGGACCCGGGGCCCCGCGGGGCTGGGG |
| CCCCAGGGATCCCGCCCAGGGTGCCCC | GGGGCACCCTGGGCGGGATCCCTGGGG |
| CCCCATTATCCCCACCCCTTTGGTCCC | GGGACCAAAGGGGTGGGGATAATGGGG |
| CCCCCACCCTCTGCCCCCATCCCACCC | GGGTGGGATGGGGGCAGAGGGTGGGGG |
| CCCCCACCGCCCCCCTCCCTTCATCCC | GGGATGAAGGGAGGGGGGCGGTGGGGG |
| CCCCCAGCCCGTCCCCACCCCCACCCC | GGGGTGGGGGTGGGGACGGGCTGGGGG |
| CCCCCATACCCAGCCCCCAACAAACCC | GGGTTTGTTGGGGGCTGGGTATGGGGG |
| CCCCCCACCCCCACCCCCCCCCGCCCC | GGGGCGGGGGGGGGTGGGGGTGGGGGG |
| CCCCCCCACCCCAACACCCCCTCCCCC | GGGGGAGGGGGTGTTGGGGTGGGGGGG |
| CCCCCCCAGCCCTCCCTGCCCGGACCC | GGGTCCGGGCAGGGAGGGCTGGGGGGG |
| CCCCCCCCCGGGCCCCCCCCGAGCCCC | GGGGCTCGGGGGGGGCCCGGGGGGGGG |
| CCCCCCTCGCCCCGGCCTCCCCCGCCC | GGGCGGGGGAGGCCGGGGCGAGGGGGG |
| CCCCCGCCCCGCGCCCCGTCCCGCCCC | GGGGCGGGACGGGGCGCGGGGCGGGGG |
| CCCCCGGCCCATGCCTCCCACCCCCCC | GGGGGGGTGGGAGGCATGGGCCGGGGG |
| CCCCCTCCCCATTCCCCTCCCCACCCC | GGGGTGGGGAGGGGAATGGGGAGGGGG |
| CCCCCTCCTCCCTTCCCTCCTTCTCCC | GGGAGAAGGAGGGAAGGGAGGAGGGGG |
| CCCCGCAGGCCCCCTGCCCAGGCCCCC | GGGGGCCTGGGCAGGGGGCCTGCGGGG |
| CCCCGCCATCCCGGGGGCCCGCCCCCC | GGGGGGCGGGCCCCCGGGATGGCGGGG |
| CCCCGCCCCGACCCCGCCCATTGGCCC | GGGCCAATGGGCGGGGTCGGGGCGGGG |
| CCCCGCCCTACCCAGGGCCCCGCCCCC | GGGGGCGGGGCCCTGGGTAGGGCGGGG |
| CCCCGCCGCCCGGCCCCTTTCTCGCCC | GGGCGAGAAAGGGGCCGGGCGGCGGGG |
| CCCCGCTGCCCAGGCGCCCCGCCACCC | GGGTGGCGGGGCGCCTGGGCAGCGGGG |
| CCCCGGCCCCGCCCCGGCCCGCCCCCC | GGGGGGCGGGCCGGGGCGGGGCCGGGG |
| CCCCGGCCCCGCCCGCCCGGCCCGCCC | GGGCGGGCCGGGCGGGCGGGGCCGGGG |
| CCCCGGCCCCGCCGCCCACCCCGGCCC | GGGCCGGGGTGGGCGGCGGGGCCGGGG |
| CCCCGTCCCTGTCCCCCAACTCACCCC | GGGGTGAGTTGGGGGACAGGGACGGGG |
| CCCCGTCTTCCCTGCCCGGCCTCCCCC | GGGGGAGGCCGGGCAGGGAAGACGGGG |
| CCCCTCCCCCAGCTCCCCTCCCCTCCC | GGGAGGGGAGGGGAGCTGGGGGAGGGG |
| CCCCTCCCCCCTGTCCCGGCTCGGCCC | GGGCCGAGCCGGGACAGGGGGGAGGGG |
| CCCCTCCCGTCCCTACCCTCTGCCCCC | GGGGGCAGAGGGTAGGGACGGGAGGGG |
| CCCCTCCCTCCCGGCAGCCCCAGCCCC | GGGGCTGGGGCTGCCGGGACGGAGGGG |
| CCCCTCGGACCCCGCCCCCGCGGCCCC | GGGGCCGCGGGGGCGGGGTCCGAGGGG |
| CCCCTCGGCCCCGGCCCCCTCCCGCCC | GGGCGGGAGGGGGCCGGGGCCGAGGGG |
| CCCCTCTGTCCCCACCCAACCCCGCCC | GGGCGGGGTTGGGTGGGGACAGAGGGG |
| CCCCTGCCCCCCGCCCCGCACAACCCC | GGGGTTGTGCGGGGCGGGGGGCAGGGG |
| CCCCTGCCCGCCCTGGCCCAGCTGCCC | GGGCAGCTGGGCCAGGGCGGGCAGGGG |
| CCCCTTCCCTCACACCCTGCCTCGCCC | GGGCGAGGCAGGGTGTGAGGGAAGGGG |
| CCCGAGTCCCACGCTGTCCCCATGCCC | GGGCATGGGGACAGCGTGGGACTCGGG |
| CCCGATCCCCCATTCCCGCGCGCCCCC | GGGGGCGCGGGCGAATGGGGGATCGGG |
| CCCGCAGGCTCCCTGCCCGCCTTCCCC | GGGGAAGGCGGGCAGGGAGCCTGCGGG |
| CCCGCCATCCCCGTGCCCATTAAGCCC | GGGCTTAATGGGCACGGGGATGGCGGG |
| CCCGCCCCATGGAGCCCCCTGCCGCCC | GGGCGGCAGGGGGCTCCATGGGGCGGG |
| CCCGCCCCGCCCAGTGCCCCACCACCC | GGGTGGTGGGGCACTGGGCGGGGCGGG |
| CCCGCCCCGCCCTGGCCCACCGGCCCC | GGGGCCGGTGGGCCAGGGCGGGGCGGG |
| CCCGCCCGCCCGACCCCGGCCCGACCC | GGGTCGGGCCGGGGTCGGGCGGGCGGG |
| CCCGCCCGCCCGCTCACCCGCTCACCC | GGGTGAGCGGGTGAGCGGGCGGGCGGG |
| CCCGGCACCGCCCCAGCCCCCGCACCC | GGGTGCGGGGGCTGGGGCGGTGCCGGG |
| CCCGGCAGCCCAGACTGGCCCTTCCCC | GGGGAAGGGCCAGTCTGGGCTGCCGGG |
| CCCGGCCCCTCCCTCCGCCCCACCCCC | GGGGGTGGGGCGGAGGGAGGGGCCGGG |
| CCCGGCTTCCCTGACAGCCCCTACCCC | GGGGTAGGGGCTGTCAGGGAAGCCGGG |
| CCCGGGCCCCCGGGCCCCGCCGCCCCC | GGGGGCGGCGGGGCCCGGGGGCCCGGG |
| CCCGTGGCCCACCACCTCCCAAGGCCC | GGGCCTTGGGAGGTGGTGGGCCACGGG |
| CCCGTTTCCCTCACCCCACCCCCTCCC | GGGAGGGGGTGGGGTGAGGGAAACGGG |
| CCCTAGTCCTCCCGCCGCCCGTTCCCC | GGGGAACGGGCGGCGGGAGGACTAGGG |
| CCCTCACTCTCCCGCCCCTACCTTCCC | GGGAAGGTAGGGGCGGGAGAGTGAGGG |
| CCCTCCAGCCCCTGGCCCTGCAGGCCC | GGGCCTGCAGGGCCAGGGGCTGGAGGG |
| CCCTCCCCCACCCCGAGCCCTGGCCCC | GGGGCCAGGGCTCGGGGTGGGGGAGGG |
| CCCTCCCGCTTGCTCCCCAGCCCACCC | GGGTGGGCTGGGGAGCAAGCGGGAGGG |
| CCCTCCCGGGAGCCCCCCCTTTAGCCC | GGGCTAAAGGGGGGGCTCCCGGGAGGG |
| CCCTCCCTGTCCCTGCCCTCCTCCCCC | GGGGGAGGAGGGCAGGGACAGGGAGGG |
| CCCTCTCCCAAAGGACCCCCGAAGCCC | GGGCTTCGGGGGTCCTTTGGGAGAGGG |
| CCCTCTCCCCCACCCTCCCCCCACCCC | GGGGTGGGGGGAGGGTGGGGGAGAGGG |
| CCCTCTTCCCCAGGCCCACAAGCCCCC | GGGGGCTTGTGGGCCTGGGGAAGAGGG |
| CCCTGCAGACCCCGCAGGGCCCAGCCC | GGGCTGGGCCCTGCGGGGTCTGCAGGG |
| CCCTGCCCTGCCCTACCCTACTCTCCC | GGGAGAGTAGGGTAGGGCAGGGCAGGG |
| CCCTGCTACCCCTTGTCTCCCCAGCCC | GGGCTGGGGAGACAAGGGGTAGCAGGG |
| CCCTGGCCCCTCCCTCCCAGCCCACCC | GGGTGGGCTGGGAGGGAGGGGCCAGGG |
| CCCTGGGCAGCCCCCGGCCCTGGCCCC | GGGGCCAGGGCCGGGGGCTGCCCAGGG |
| CCCTTCCCCCCCGCCCCCCACCGTCCC | GGGACGGTGGGGGGCGGGGGGGAAGGG |
| CCCTTTCCCCGCCAACCCCCGCCCCCC | GGGGGGCGGGGGTTGGCGGGGAAAGGG |
| GGGAACGGGGGGTTGGGTGCTCGAGGG | CCCTCGAGCACCCAACCCCCCGTTCCC |
| GGGAAGGAGGGAGGGAGGGAGGGAGGG | CCCTCCCTCCCTCCCTCCCTCCTTCCC |
| GGGAAGGGCAGGGTGGGCACCAAGGGG | CCCCTTGGTGCCCACCCTGCCCTTCCC |
| GGGAAGGGGTGGGAGGCAGAGGGAGGG | CCCTCCCTCTGCCTCCCACCCCTTCCC |
| GGGAATTGAGGGTGAGGGGTTTGGGGG | CCCCCAAACCCCTCACCCTCAATTCCC |
| GGGACGAGGGAGGCGGAGGGAGGAGGG | CCCTCCTCCCTCCGCCTCCCTCGTCCC |
| GGGAGAGCAGGGCTGGGGGCCACCGGG | CCCGGTGGCCCCCAGCCCTGCTCTCCC |
| GGGAGCGGGGCGCAGGGCGTCTAGGGG | CCCCTAGACGCCCTGCGCCCCGCTCCC |
| GGGAGCTGGGCCTGGCGGGAAGCAGGG | CCCTGCTTCCCGCCAGGCCCAGCTCCC |
| GGGAGGACGGGAGGGAGGGCTTGCGGG | CCCGCAAGCCCTCCCTCCCGTCCTCCC |
| GGGAGGAGGGGCGGGGGCGCGGAGGGG | CCCCTCCGCGCCCCCGCCCCTCCTCCC |
| GGGAGGCGGCGGGGCGGGCCGCCGGGG | CCCCGGCGGCCCGCCCCGCCGCCTCCC |
| GGGAGGCGGGAAGGGTGGGGGGAGGGG | CCCCTCCCCCCACCCTTCCCGCCTCCC |
| GGGAGGGAGAGGGTGGGTAGTACTGGG | CCCAGTACTACCCACCCTCTCCCTCCC |
| GGGAGGGAGGGTGGGGGAGGACGAGGG | CCCTCGTCCTCCCCCACCCTCCCTCCC |
| GGGAGGGCCTGGGGTGGGGGCCAGGGG | CCCCTGGCCCCCACCCCAGGCCCTCCC |
| GGGAGGGCGGGAAAGGAAGGGGCGGGG | CCCCGCCCCTTCCTTTCCCGCCCTCCC |
| GGGAGGGGAGGGGGGACTTGGGGGGGG | CCCCCCCCAAGTCCCCCCTCCCCTCCC |
| GGGAGGGGAGGGGGTCCCGGGCCGGGG | CCCCGGCCCGGGACCCCCTCCCCTCCC |
| GGGATCGGGGAGGGCGGGGGCTTCGGG | CCCGAAGCCCCCGCCCTCCCCGATCCC |
| GGGATGGGGTGGCTGGGCTGGGCAGGG | CCCTGCCCAGCCCAGCCACCCCATCCC |
| GGGCAGGGGAGAGGGAGGGGGCTGGGG | CCCCAGCCCCCTCCCTCTCCCCTGCCC |
| GGGCAGGGGTGAGAGGGCTTGGGGGGG | CCCCCCCAAGCCCTCTCACCCCTGCCC |
| GGGCCGCGGGCCCCGGGTGCCTCGGGG | CCCCGAGGCACCCGGGGCCCGCGGCCC |
| GGGCCGCGGGGCTCGGGCCTATTGGGG | CCCCAATAGGCCCGAGCCCCGCGGCCC |
| GGGCCGGGCGTCGGGGGCCCGGGCGGG | CCCGCCCGGGCCCCCGACGCCCGGCCC |
| GGGCCTAAGGGAGGGGGAAAGCGAGGG | CCCTCGCTTTCCCCCTCCCTTAGGCCC |
| GGGCGAGGGGCTGCAGGGCACGTAGGG | CCCTACGTGCCCTGCAGCCCCTCGCCC |
| GGGCGCAGGGGGGTGGGTGCGGAGGGG | CCCCTCCGCACCCACCCCCCTGCCCCC |
| GGGCGCATGGGGCGGGAGGGGGCCGGG | CCCGGCCCCCTCCCGCCCCATGCGCCC |
| GGGCGGCGCCGGGTAGGGGGGCCAGGG | CCCTGGCCCCCCTACCCGGCGCCGCCC |
| GGGCGGCGGGGGCAGCGGGGCCTTGGG | CCCAAGGCCCCGCTGCCCCCGCCGCCC |
| GGGCGGGAGGGGGGCGGGGCTCGTGGG | CCCACGAGCCCCGCCCCCCTCCCGCCC |
| GGGCGGGGGTGCCGGGGTGGTGTGGGG | CCCCACACCACCCCGGCACCCCCGCCC |
| GGGCGGGTGAGTGCGGGGTCAGGAGGG | CCCTCCTGACCCCGCACTCACCCGCCC |
| GGGCGTGGTAGGGTCCGGGCTCAGGGG | CCCCTGAGCCCGGACCCTACCACGCCC |
| GGGCTGAGGGGTGGGGGAGGGCAGGGG | CCCCTGCCCTCCCCCACCCCTCAGCCC |
| GGGCTGGGAGCTGGGGGCTGGGTGGGG | CCCCACCCAGCCCCCAGCTCCCAGCCC |
| GGGCTGGGCTGGGCTGGGCTGGCGGGG | CCCCGCCAGCCCAGCCCAGCCCAGCCC |
| GGGGAAAGGGGAAAAGGGGAAGGGGGG | CCCCCCTTCCCCTTTTCCCCTTTCCCC |
| GGGGAAGGGGATGAGGGCAAGGGTGGG | CCCACCCTTGCCCTCATCCCCTTCCCC |
| GGGGACAGGGGGTGGTCGGGGCATGGG | CCCATGCCCCGACCACCCCCTGTCCCC |
| GGGGAGAGAGGGCTGGGGGTGGCTGGG | CCCAGCCACCCCCAGCCCTCTCTCCCC |
| GGGGAGAGGGAAAGGCAGGGGGCGGGG | CCCCGCCCCCTGCCTTTCCCTCTCCCC |
| GGGGAGGAGCGGGGAGGCGGGGCGGGG | CCCCGCCCCGCCTCCCCGCTCCTCCCC |
| GGGGAGGAGTGGGGACATGGGGGAGGG | CCCTCCCCCATGTCCCCACTCCTCCCC |
| GGGGAGTGGGCTCGGCGGGGCCGGGGG | CCCCCGGCCCCGCCGAGCCCACTCCCC |
| GGGGAGTGGGGGTGCCGCAGGGTCGGG | CCCGACCCTGCGGCACCCCCACTCCCC |
| GGGGATAGGGAAGGGAGGGGAAAGGGG | CCCCTTTCCCCTCCCTTCCCTATCCCC |
| GGGGCCAGAGGGCAGCAGGGGAGGGGG | CCCCCTCCCCTGCTGCCCTCTGGCCCC |
| GGGGCGAGAGGGTGGGGAGGGGCGGGG | CCCCGCCCCTCCCCACCCTCTCGCCCC |
| GGGGCGCCGGGACAGGGGCTGGGTGGG | CCCACCCAGCCCCTGTCCCGGCGCCCC |
| GGGGCGGGACGGGGCGGGACTGCGGGG | CCCCGCAGTCCCGCCCCGTCCCGCCCC |
| GGGGCGGGCCGGGGCTGGGAGAGAGGG | CCCTCTCTCCCAGCCCCGGCCCGCCCC |
| GGGGCGGGCGGGGCCGGGACCTGGGGG | CCCCCAGGTCCCGGCCCCGCCCGCCCC |
| GGGGCGGGGAGGGAGGCGGGGTTGGGG | CCCCAACCCCGCCTCCCTCCCCGCCCC |
| GGGGCTCAGCGGGCTGGCGGGAGGGGG | CCCCCTCCCGCCAGCCCGCTGAGCCCC |
| GGGGCTCTGGGCGGGGGCCGGGCCGGG | CCCGGCCCGGCCCCCGCCCAGAGCCCC |
| GGGGCTGGGGAGGGGCCGGGGTCAGGG | CCCTGACCCCGGCCCCTCCCCAGCCCC |
| GGGGCTGGGGGCGGGGGCCCTCCTGGG | CCCAGGAGGGCCCCCGCCCCCAGCCCC |
| GGGGCTTGTTGGGGATGCGGGCCAGGG | CCCTGGCCCGCATCCCCAACAAGCCCC |
| GGGGGAAGGGGGCCTCAGGGGCCAGGG | CCCTGGCCCCTGAGGCCCCCTTCCCCC |
| GGGGGAAGGGGGTAGGGGCCGCGCGGG | CCCGCGCGGCCCCTACCCCCTTCCCCC |
| GGGGGAAGTGGGGGCCAGGGCCCCGGG | CCCGGGGCCCTGGCCCCCACTTCCCCC |
| GGGGGACTGGGTGGGAAGGGTGGTGGG | CCCACCACCCTTCCCACCCAGTCCCCC |
| GGGGGAGCGGGGGCAGGGCCGGCGGGG | CCCCGCCGGCCCTGCCCCCGCTCCCCC |
| GGGGGAGGGGACCGAGGGCCCGGGGGG | CCCCCCGGGCCCTCGGTCCCCTCCCCC |
| GGGGGATTAGGGCGAGGGAAGGTGGGG | CCCCACCTTCCCTCGCCCTAATCCCCC |
| GGGGGCAGTTGGGGCTGGGGGTGGGGG | CCCCCACCCCCAGCCCCAACTGCCCCC |
| GGGGGCCGAGGGGAGGGAGGAGCGGGG | CCCCGCTCCTCCCTCCCCTCGGCCCCC |
| GGGGGCCGGGGAGGGGGGCGGAGGGGG | CCCCCTCCGCCCCCCTCCCCGGCCCCC |
| GGGGGCGGGGGCGGGCGTGGGGCGGGG | CCCCGCCCCACGCCCGCCCCCGCCCCC |
| GGGGGGAGGGGGAGAGGGGATCCAGGG | CCCTGGATCCCCTCTCCCCCTCCCCCC |
| GGGGGGCCGGGCGAGGGCGGACGCGGG | CCCGCGTCCGCCCTCGCCCGGCCCCCC |
| GGGGGGCGGGAGCTGGGGGGCCCCGGG | CCCGGGGCCCCCCAGCTCCCGCCCCCC |
| GGGGGGCGGGGGGCGGGGGGCGTGGGG | CCCCACGCCCCCCGCCCCCCGCCCCCC |
| GGGGGGCTGGGGGGCGGGGGCCCGGGG | CCCCGGGCCCCCGCCCCCCAGCCCCCC |
| GGGGGGGAGGGCAGGGGCCCCTGTGGG | CCCACAGGGGCCCCTGCCCTCCCCCCC |
| GGGGGGGCTCCAGTGGGGCCACCGGGG | CCCCGGTGGCCCCACTGGAGCCCCCCC |
| GGGGGGGGGGAGGTGGGAGGGCAAGGG | CCCTTGCCCTCCCACCTCCCCCCCCCC |
| GGGGGGTGGGGTGGGGGTTGGGTGGGG | CCCCACCCAACCCCCACCCCACCCCCC |
| GGGGGTGATGGGTGGGGTCACGGGGGG | CCCCCCGTGACCCCACCCATCACCCCC |
| GGGGGTGGGGACGTGGGGGCGGGTGGG | CCCACCCGCCCCCACGTCCCCACCCCC |
| GGGGGTGGGGCGGGGGGCGCCGCGGGG | CCCCGCGGCGCCCCCCGCCCCACCCCC |
| GGGGGTGGGGGCGGGGGCAGGGCCGGG | CCCGGCCCTGCCCCCGCCCCCACCCCC |
| GGGGGTGGGGTGAAGGGGGAAGAGGGG | CCCCTCTTCCCCCTTCACCCCACCCCC |
| GGGGGTTGGGAAGGGAGGGAGAGGGGG | CCCCCTCTCCCTCCCTTCCCAACCCCC |
| GGGGTCATGAGGGGGTGGGGGCTGGGG | CCCCAGCCCCCACCCCCTCATGACCCC |
| GGGGTCGGGCCATGGGGGCGCCTGGGG | CCCCAGGCGCCCCCATGGCCCGACCCC |
| GGGGTGGGGAGGGAGGCGGGGCTGGGG | CCCCAGCCCCGCCTCCCTCCCCACCCC |
| GGGGTGGGGAGGGAGGCGGGGTTGGGG | CCCCAACCCCGCCTCCCTCCCCACCCC |
| GGGGTTGGGTGATGGGGAGTAGGGGGG | CCCCCCTACTCCCCATCACCCAACCCC |
| GGGTAGGAGAGGGTGTGGGATGGTGGG | CCCACCATCCCACACCCTCTCCTACCC |
| GGGTCCGGGTGCTGCAGGGTGTCCGGG | CCCGGACACCCTGCAGCACCCGGACCC |
| GGGTCCTGAAGGGCGGGCCCCTAGGGG | CCCCTAGGGGCCCGCCCTTCAGGACCC |
| GGGTCGTGGGGCGGAGGGAAGAGCGGG | CCCGCTCTTCCCTCCGCCCCACGACCC |
| GGGTGCAAAAGGGAAAGGGGGGTGGGG | CCCCACCCCCCTTTCCCTTTTGCACCC |
| GGGTGCAGCGGGGTCGGGTCTTCGGGG | CCCCGAAGACCCGACCCCGCTGCACCC |
| GGGTGCTGGGCACTGGGAGGTGGCGGG | CCCGCCACCTCCCAGTGCCCAGCACCC |
| GGGTGCTGGGCATTGGGAGGTGGCGGG | CCCGCCACCTCCCAATGCCCAGCACCC |
| GGGTGCTGGGGAGGGGGAAGGGCAGGG | CCCTGCCCTTCCCCCTCCCCAGCACCC |
| GGGTGGAGAGGGTAAAGGGAGGCAGGG | CCCTGCCTCCCTTTACCCTCTCCACCC |
| GGGTGGCGGGGGCAGGGCCCGTGGGGG | CCCCCACGGGCCCTGCCCCCGCCACCC |
| GGGTGGGGGGTGGTTCCGGGTCCAGGG | CCCTGGACCCGGAACCACCCCCCACCC |
| CCCAAAACACCCTCCCTGTCCCAACCCC | GGGGTTGGGACAGGGAGGGTGTTTTGGG |
| CCCACAGGCCCCGCCCCCCCCCCCGCCC | GGGCGGGGGGGGGGGCGGGGCCTGTGGG |
| CCCACCCACCCCCTCAGATCCCCACCCC | GGGGTGGGGATCTGAGGGGGTGGGTGGG |
| CCCACCCCACCCCCAGCAACCCCGTCCC | GGGACGGGGTTGCTGGGGGTGGGGTGGG |
| CCCACGTTCCCCTCTCCCAGCCTCCCCC | GGGGGAGGCTGGGAGAGGGGAACGTGGG |
| CCCACTGCCCAATCCCACCCTCGACCCC | GGGGTCGAGGGTGGGATTGGGCAGTGGG |
| CCCACTGCCCTTCCCACCCCCACAGCCC | GGGCTGTGGGGGTGGGAAGGGCAGTGGG |
| CCCAGAGGTGCCCGGGCCCGCGGGACCC | GGGTCCCGCGGGCCCGGGCACCTCTGGG |
| CCCAGCCCTGCCTCCCCCACCCTCGCCC | GGGCGAGGGTGGGGGAGGCAGGGCTGGG |
| CCCAGCCGCCCCACCCTCCCGTGGGCCC | GGGCCCACGGGAGGGTGGGGCGGCTGGG |
| CCCAGCTCGGCCCCACCCGCTCCGCCCC | GGGGCGGAGCGGGTGGGGCCGAGCTGGG |
| CCCAGGAGCGCCCTACCCTCTGGGGCCC | GGGCCCCAGAGGGTAGGGCGCTCCTGGG |
| CCCAGGCCCCCCTACCCCTCCTATCCCC | GGGGATAGGAGGGGTAGGGGGGCCTGGG |
| CCCAGGCGTCCCGCCTTCCCATGCCCCC | GGGGGCATGGGAAGGCGGGACGCCTGGG |
| CCCAGTGCCCCCATCGACCCCCAGGCCC | GGGCCTGGGGGTCGATGGGGGCACTGGG |
| CCCAGTGTGCCCCGCCCACCCATGGCCC | GGGCCATGGGTGGGCGGGGCACACTGGG |
| CCCATCCAGGCCCCCTCTGCCCTGCCCC | GGGGCAGGGCAGAGGGGGCCTGGATGGG |
| CCCATTCCCCTCTTCTCCCACTTCTCCC | GGGAGAAGTGGGAGAAGAGGGGAATGGG |
| CCCCACACCCCCATCCATCCCATTTCCC | GGGAAATGGGATGGATGGGGGTGTGGGG |
| CCCCACCCCCCACCCCCTCCCCCCCCC | GGGGGGGGGGAGGGGGTGGGGGGTGGGG |
| CCCCACGAGCCCCCTGCCCGCTGCTCC | GGGAGCAGCGGGCAGGGGGCTCGTGGGG |
| CCCCAGATCCCTCAGGCCCACACGCCCC | GGGGCGTGTGGGCCTGAGGGATCTGGGG |
| CCCCAGCCCGCCCCCAGCCCTCCCGCCC | GGGCGGGAGGGCTGGGGGCGGGCTGGGG |
| CCCCATGGCGCCCAGCACCCCACAGCCC | GGGCTGTGGGGTGCTGGGCGCCATGGGG |
| CCCCCAAACCCCTCCCCACCCCTCCCCC | GGGGGAGGGGTGGGGAGGGGTTTGGGGG |
| CCCCCACAGCCCAGCCCAGCCCAGGCCC | GGGCCTGGGCTGGGCTGGGCTGTGGGGG |
| CCCCCACATCCCCATCCCCCAACCACCC | GGGTGGTTGGGGGATGGGGATGTGGGGG |
| CCCCCAGCCCTCCTCCCCGGCACCTCCC | GGGAGGTGCCGGGGAGGAGGGCTGGGGG |
| CCCCCCTCCCCAAACTAACCCCCTGCCC | GGGCAGGGGGTTAGTTTGGGGAGGGGGG |
| CCCCCGCATGCCCCGTCCCTTAGCTCCC | GGGAGCTAAGGGACGGGGCATGCGGGGG |
| CCCCCGCCCCTTGACATCCCAGACTCCC | GGGAGTCTGGGATGTCAAGGGGCGGGGG |
| CCCCCGCGCCCCCAGTCCCCGCGTCCCC | GGGGACGCGGGGACTGGGGGCGCGGGGG |
| CCCCCGGCCCCGCCCCCGCCCCCGGCCC | GGGCCGGGGGCGGGGGCGGGGCCGGGGG |
| CCCCCTCATCCCGTCTTCCCCTCCTCCC | GGGAGGAGGGGAAGACGGGATGAGGGGG |
| CCCCCTCCACCCCCACCCTGAGGCTCCC | GGGAGCCTCAGGGTGGGGGTGGAGGGGG |
| CCCCCTCCCCATCTCCCCAAATCCACCC | GGGTGGATTTGGGGAGATGGGGAGGGGG |
| CCCCGCAGCCCCGCCCCCGGCCCTCCCC | GGGGAGGGCCGGGGGCGGGGCTGCGGGG |
| CCCCGCCCCTGCCCTACCCCCAGCCCCC | GGGGGCTGGGGGTAGGGCAGGGGCGGGG |
| CCCCGCCCGCTGTCGCCCATCCCGTCCC | GGGACGGGATGGGCGACAGCGGGCGGGG |
| CCCCGGCCCCGCCCGGCCCCCGGCCCCC | GGGGGCCGGGGGCCGGGCGGGGCCGGGG |
| CCCCTCCAAGCCCCGCCCTCTCTAGCCC | GGGCTAGAGAGGGCGGGGCTTGGAGGGG |
| CCCCTCCCCGTCTAGCCCCCTCCTCCCC | GGGGAGGAGGGGGCTAGACGGGGAGGGG |
| CCCCTGAGCCCCCAGCCCCTGAGCCCCC | GGGGGCTCAGGGGCTGGGGGCTCAGGGG |
| CCCCTTCCCTGCCCCCCCCACCCTTCCC | GGGAAGGGTGGGGGGGGCAGGGAAGGGG |
| CCCCTTGACCCCAACCAAGCCCCGCCCC | GGGGCGGGGCTTGGTTGGGGTCAAGGGG |
| CCCCTTTCCCACCCACCCCAAGAAGCCC | GGGCTTCTTGGGGTGGGTGGGAAAGGGG |
| CCCGACTGCCCCACTCTCCCCGGCCCCC | GGGGGCCGGGGAGAGTGGGGCAGTCGGG |
| CCCGCCCCCGCCCCCCTGCCCCGGCCCC | GGGGCCGGGGCAGGGGGGCGGGGGCGGG |
| CCCGCCCCCTCCCTCCAGCCCGCCCCCC | GGGGGGCGGGCTGGAGGGAGGGGGCGGG |
| CCCGCCCCCTGCCCCCCCTCGCTCCCCC | GGGGGAGCGAGGGGGGGCAGGGGGCGGG |
| CCCGCCCCGCCGGCCCCCGCCGGCCCCC | GGGGGCCGGCGGGGGCCGGCGGGGCGGG |
| CCCGCCGTGCCCCGCCCGGCCCGCGCCC | GGGCGCGGGCCGGGCGGGGCACGGCGGG |
| CCCGGCAGCCCTCGCCCCTGTGAGGCCC | GGGCCTCACAGGGGCGAGGGCTGCCGGG |
| CCCGGCCCCCGCCCCCACCCCCGCCCCC | GGGGGCGGGGGTGGGGGCGGGGGCCGGG |
| CCCGGTTAACCCCTGCACCCCAGTCCCC | GGGGACTGGGGTGCAGGGGTTAACCGGG |
| CCCTCCACCCCGCTCCCCTCCTGCCCCC | GGGGGCAGGAGGGGAGCGGGGTGGAGGG |
| CCCTCCCATCCCTCCCCCTAGCTTGCCC | GGGCAAGCTAGGGGGAGGGATGGGAGGG |
| CCCTCCCCATCCCCCCAGCCCAGGCCCC | GGGGCCTGGGCTGGGGGGATGGGGAGGG |
| CCCTCCCCTTCCCTGCCCTCCCCTGCCC | GGGCAGGGGAGGGCAGGGAAGGGGAGGG |
| CCCTCCCGACCCACCCCGTCCCCACCCC | GGGGTGGGGACGGGGTGGGTCGGGAGGG |
| CCCTCCCTGACCCCCACCCTGGCCCCCC | GGGGGGCCAGGGTGGGGGTCAGGGAGGG |
| CCCTCCCTGGCCCCCGTCCCACTTCCC | GGGAAGTGGGACCGGGGGCCAGGGAGGG |
| CCCTCCTCCCCACACCCCTCCCAGTCCC | GGGACTGGGAGGGGTGTGGGGAGGAGGG |
| CCCTCCTTAGCCCTCCCCCCTCCCTCCC | GGGAGGGAGGGGGGAGGGCTAAGGAGGG |
| CCCTCCTTCGCCCCGGGGCCCCCTTCCC | GGGAAGGGGGCCCCGGGGCGAAGGAGGG |
| CCCTCGCCCCCCGCCCTCCCCGCATCCC | GGGATGCGGGGAGGGCGGGGGGCGAGGG |
| CCCTCTGCACCCAAATATCCCGACACCC | GGGTGTCGGGATATTTGGGTGCAGAGGG |
| CCCTGCCACCCCACTGCCCCGCTAACCC | GGGTTAGCGGGGCAGTGGGGTGGCAGGG |
| CCCTGGCCCCAGGGACCCCAGCTCCCCC | GGGGGAGCTGGGGTCCCTGGGGCCAGGG |
| CCCTGGCTCCCTTCGTTCCCCCCACCCC | GGGGTGGGGGGAACGAAGGGAGCCAGGG |
| CCCTGTCCCTCCCAACCCCCGGGCTCCC | GGGAGCCCGGGGGTTGGGAGGGACAGGG |
| CCCTTCCCCCACACTGTCCCTCACTCCC | GGGAGTGAGGGACAGTGTGGGGGAAGGG |
| CCCTTGCCAGCCCCAGTTCCCACCCCCC | GGGGGGTGGGAACTGGGGCTGGCAAGGG |
| GGGAAGAAGGGATATTTGGGGCTCTGGG | CCCAGAGCCCCAAATATCCCTTCTTCCC |
| GGGAAGCGGGCGGGGATGGGAGGGAGGG | CCCTCCCTCCCATCCCCGCCCGCTTCCC |
| GGGACACGCGGGGCTAACGGGGGCCGGG | CCCGGCCCCCGTTAGCCCCGCGTGTCCC |
| GGGACCCTGGGAGGGGCGGGGGTGGGGG | CCCCCACCCCCGCCCCTCCCAGGGTCCC |
| GGGACCCTGGGGCTCTGGGAGAGATGGG | CCCATCTCTCCCAGAGCCCCAGGGTCCC |
| GGGACTGGGGGTGGAGGGGTGCGGGGGG | CCCCCCGCACCCCTCCACCCCCAGTCCC |
| GGGAGCAGGGCCAGAGGGGGCCCGTGGG | CCCACGGGCCCCCTCTGGCCCTGCTCCC |
| GGGAGCGGGCAAAGATGGGATCAGGGGG | CCCCCTGATCCCATCTTTGCCCGCTCCC |
| GGGAGCTAGGGGACGGGGAATGCGGGGG | CCCCCGCATTCCCCGTCCCCTAGCTCCC |
| GGGAGCTGGCGGGCGGGGCTGGCAGGGG | CCCCTGCCAGCCCCGCCCGCCAGCTCCC |
| GGGAGGAACAGGGCAGGGGAGGGAAGGG | CCCTTCCCTCCCCTGCCCTGTTCCTCCC |
| GGGAGGAGGGGAAGGAAGGGAAGGTGGG | CCCACCTTCCCTTCCTTCCCCTCCTCCC |
| GGGAGGGGCTGGGGCGTGGGAGGTGGGG | CCCCACCTCCCACGCCCCAGCCCCTCCC |
| GGGAGGTGAGGGCAGGGCAGGGCGGGGG | CCCCCGCCCTGCCCTGCCCTCACCTCCC |
| GGGAGGTGGGAGCCTGGGAACAGTGGGG | CCCCACTGTTCCCAGGCTCCCACCTCCC |
| GGGATAGGCTGGGAGCCTGGGGGCAGGG | CCCTGCCCCCAGGCTCCCAGCCTATCCC |
| GGGATATGGGGGCGGGGGCGGGGCAGGG | CCCTGCCCCGCCCCCGCCCCCATATCCC |
| GGGATGGGAGGCGGAGGGAGGGCGCGGG | CCCGCGCCCTCCCTCCGCCTCCCATCCC |
| GGGATGGGTGGGGGCGGGTAGAGGGGGG | CCCCCCTCTACCCGCCCCCACCCATCCC |
| GGGATTTTTGGGCTCTCCGGGGTTCGGG | CCCGAACCCCGGAGAGCCCAAAAATCCC |
| GGGCAGAGGGGGCACGTACGGGGCTGGG | CCCAGCCCCGTACGTGCCCCCTCTGCCC |
| GGGCAGGAGAGGGAGTTGGGCAACGGGG | CCCCGTTGCCCAACTCCCTCTCCTGCCC |
| GGGCCAGGAGGGAGGGAGGGGAGCCGGG | CCCGGCTCCCCTCCCTCCCTCCTGGCCC |
| GGGCCAGGGCGGCACTGGGCGCTGAGGG | CCCTCAGCGCCCAGTGCCGCCCTGGCCC |
| GGGCCCTGGGCTGGGGGGGCCCCCAGGG | CCCTGGGGGCCCCCCCAGCCCAGGGCCC |
| GGGCCGCCGCGGGAGCCCGGGGATCGGG | CCCGATCCCCGGGCTCCCGCGGCGGCCC |
| GGGCCGCGGCGGGCGGGGCTCCGGCGGG | CCCGCCGGAGCCCCGCCCGCCGCGGCCC |
| GGGCCGGGCAGTGCAGGGCAGGGCAGGG | CCCTGCCCTGCCCTGCACTGCCCGGCCC |
| GGGCCGGGGCGGCAAGGGGCCGGGTGGG | CCCACCCGGCCCCTTGCCGCCCCGGCCC |
| GGGCCTGAGGGGCTCCGGGCTGGGAGGG | CCCTCCCAGCCCGGAGCCCCTCAGGCCC |
| GGGCCTGGGGGCTGCGGGCACCGTGGGG | CCCCACGGTGCCCGCAGCCCCCAGGCCC |
| GGGCGCGGGCGCCGCGGGAAGGGGAGGG | CCCTCCCCTTCCCGCGGCGCCCGCGCCC |
| GGGCGGAAAGGGCGGGGGCAGAGGTGGG | CCCACCTCTGCCCCCGCCCTTTCCGCCC |
| GGGCGGCGGCGGGGCCTGGGGTCTGGGG | CCCCAGACCCCAGGCCCCGCCGCCGCCC |
| GGGCTGCGGGCGGCTGGGGCACCGCGGG | CCCGCGGTGCCCCAGCCGCCCGCAGCCC |
| GGGCTGGGAGCAGGCGGGGCCGTGGGGG | CCCCCACGGCCCCGCCTGCTCCCAGCCC |
| GGGCTGGGAGCCAGAGGGCCTTAAAGGG | CCCTTTAAGGCCCTCTGGCTCCCAGCCC |
| GGGGAAGAGGGGCATGTGGGGCAGGGGG | CCCCCTGCCCCACATGCCCCTCTTCCCC |
| GGGGAAGGGGGGGGGGGCCGGGGCAGGG | CCCTGCCCCGGCCCCCCCCCCCTTCCCC |
| GGGGAGGGCAGGGGAGGGCAGGGCAGGG | CCCTGCCCTGCCCTCCCCTGCCCTCCCC |
| GGGGAGGGGGCGGGTGGGAGCGGAGGGG | CCCCTCCGCTCCCACCCGCCCCCTCCCC |
| GGGGAGGGGGCTGGGCAGGGGGGCTGGG | CCCAGCCCCCCTGCCCAGCCCCCTCCCC |
| GGGGAGGGGTGGGAAAGGGTGGGCTGGG | CCCAGCCCACCCTTTCCCACCCCTCCCC |
| GGGGAGGTGAGGGCCCTGGGGCAAAGGG | CCCTTTGCCCCAGGGCCCTCACCTCCCC |
| GGGGAGTGGGGCTGTGGGCTGGGCAGGG | CCCTGCCCAGCCCACAGCCCCACTCCCC |
| GGGGCAGGGTGGGGGGATGGGCTAGGGG | CCCCTAGCCCATCCCCCCACCCTGCCCC |
| GGGGCCCCAGGGTGGGTGGGCGGTGGGG | CCCCACCGCCCACCCACCCTGGGGCCCC |
| GGGGCCGGTGGGGAGGCCGGGGAGGGGG | CCCCCTCCCCGGCCTCCCCACCGGCCCC |
| GGGGCCTGGGGGGAGGGGCGGGGCAGGG | CCCTGCCCCGCCCCTCCCCCCAGGCCCC |
| GGGGCGGACCGGGGGACGGGGGGGTGGG | CCCACCCCCCCGTCCCCCGGTCCGCCCC |
| GGGGCGGGCGCGGCGGGGAGGGGCGGGG | CCCCGCCCCTCCCCGCCGCGCCCGCCCC |
| GGGGCGGGCGGGATGGGGGTTGGCGGGG | CCCCGCCAACCCCCATCCCGCCCGCCCC |
| GGGGCGGGGGTTGGGGGGGAGGTGAGGG | CCCTCACCTCCCCCCCAACCCCCGCCCC |
| GGGGCTGTGGGACTGGGGCCCTGAGGGG | CCCCTCAGGGCCCCAGTCCCACAGCCCC |
| GGGGGATGGGCGGGGGCGGGGATGAGGG | CCCTCATCCCCGCCCCCGCCCATCCCCC |
| GGGGGCACAGGGCTGTGGGCATGATGGG | CCCATCATGCCCACAGCCCTGTGCCCCC |
| GGGGGCAGGGGCAGGGGCGGGCGGAGGG | CCCTCCGCCCGCCCCTGCCCCTGCCCCC |
| GGGGGCTGCGGGCGGTCGGGGCTCCGGG | CCCGGAGCCCCGACCGCCCGCAGCCCCC |
| GGGGGGCCCGGGCGGGGGCGGGCGGGG | CCCCGCCCCGCCCCCGCCCGGGCCCCCC |
| GGGGGGCGGAGGGGGGAGGGGAGGGGGG | CCCCCCTCCCCTCCCCCCTCCGCCCCCC |
| GGGGGGGGTGGGTTCACAGGGCCCGGGG | CCCCGGGCCCTGTGAACCCACCCCCCCC |
| GGGGGTGGGGTGGGGAGGGGTCAGGGGG | CCCCCTGACCCCTCCCCACCCCACCCCC |
| GGGGGTTTTAGGGGCCTGGGCTGGGGGG | CCCCCCAGCCCAGGCCCCTAAAACCCCC |
| GGGGTAAGGGGGCGTTTTGGGAGCCGGG | CCCGGCTCCCAAAACGCCCCCTTACCCC |
| GGGGTCCGGGGGGAGGGGGGTTCCTGGG | CCCAGGAACCCCCCTCCCCCCGGACCCC |
| GGGGTCGAGGGCAAAGGGGAGGCGGGGG | CCCCCGCCTCCCCTTTGCCCTCGACCCC |
| GGGGTGAGAGGGGGCCCGTCGGGGCGGG | CCCGCCCGACGGGCCCCCTCTCACCCC |
| GGGGTGGGGTGGGGGGGAGTGGGATGGG | CCCATCCCACTCCCCCCCACCCCACCCC |
| GGGTAGGAGGGCTGCGGGGATCATTGGG | CCCAATGATCCCCGCAGCCCTCCTACCC |
| GGGTATCGGGTGAGGGGGCTACAATGGG | CCCATTGTAGCCCCCTCACCCGATACCC |
| GGGTCAGGTGGGGAGGGGACACCAAGGG | CCCTTGGTGTCCCCTCCCCACCTGACCC |
| GGGTCGCGGGGGCCGGGGATGGAGGGGG | CCCCCTCCATCCCCGGCCCCCGCGACCC |
| GGGTGCCAAGGGGCAGGGGCAGTGGGGG | CCCCCACTGCCCCTGCCCCTTGGCACCC |
| GGGTGCTGGGGGGAGGGGGATGGCTGGG | CCCAGCCATCCCCCTCCCCCCAGCACCC |
| GGGTTATGGGGGAGGGCACGGTAAAGGG | CCCTTTACCCTGCCCTCCCCCATAACCC |
| GGGTTTGAGGGAGTAGGGTCCAGAGGGG | CCCCTCTGGACCCTACTCCCTCAAACCC |
| CCCAAAGTCCCGCCCAGCCCTGCTGCCCC | GGGGCAGCAGGGCTGGGCGGGACTTTGGG |
| CCCACAGCCCAGCGCACCCATAGGCTCCC | GGGAGCCTATGGGTGCGCTGGGCTGTGGG |
| CCCACCGCCCCCACCCCTTCCCCAGGCCC | GGGCCTGGGGAAGGGGTGGGGGCGGTGGG |
| CCCACCTCCACCCCCGCCCCCGACATCCC | GGGATGTCGGGGGCGGGGGTGGAGGTGGG |
| CCCACGACCCCCACGGCCCCTGTGTCCCC | GGGGACACAGGGGCCGTGGGGGTCGTGGG |
| CCCACGGGCCCCCGCCCCCCTCGTCCCCC | GGGGGACGAGGGGGGCGGGGCCCCGTGGG |
| CCCACTAGCCCACCCACCCACCCTCCCCC | GGGGGAGGGTGGGTGGGTGGGCTAGTGGG |
| CCCACTCGGCCCGGCCTCTCCCCGCGCCC | GGGCGCCGGGAGAGGCCGGGCCGAGTGGG |
| CCCAGCCCCCTCCCCTCCCTTCCCCTCCC | GGGAGGGGAAGGGAGGGGAGGGGGCTGGG |
| CCCAGCTTAGCCCAGCTGACCCCAGACCC | GGGTCTGGGGTCAGCTGGGCTAAGCTGGG |
| CCCAGGACCCCTTTTCCACCCTGGGCCCC | GGGGCCCAGGGTGGAAAAGGGGTCCTGGG |
| CCCAGGCGCCCCGCCCCCCTGCCCAACCC | GGGTTGGGCAGGGGGGCGGGGCGCCTGGG |
| CCCAGGGTGCCCCGGCCCCCCCACATCCC | GGGATGTGGGGGGGCCGGGGCACCCTGGG |
| CCCAGTGCCCCCTGTACCCTCCCCGACCC | GGGTCGGGAGGGTACAGGGGGCACTGGG |
| CCCCAAATGCCCCGACCGGCCCCTCCCCC | GGGGGAGGGGCCGGTCGGGGCATTTGGGG |
| CCCCACATCTCCCCACCCCCACCCACCCC | GGGGTGGGTGGGGGTGGGGAGATGTGGGG |
| CCCCAGCAACCCTCTCCCCCAGTCGGCCC | GGGCCGACTGGGGGAGAGGGTTGCTGGGG |
| CCCCAGCGCCCCCTCCCGCCCCGTTTCCC | GGGAAACGGGGCGGGAGGGGGCGCTGGGG |
| CCCCCAAGACCCTGTGGGCCCTGCTCCCC | GGGGAGCAGGGCCCACAGGGTCTTGGGGG |
| CCCCCACCCCCCCCCACCCCCCAGGACCC | GGGTCCTGGGGGGTGGGGGGGGGTGGGGG |
| CCCCCAGACACCCCTTCCGCCCCACCCCC | GGGGGTGGGGCCGAAGGGGTGTCTGGGGG |
| CCCCCCACCCCCCCTCCCCGCCTCCTCCC | GGGAGGAGGCGGGGAGGGGGGGTGGGGGG |
| CCCCCCACCCCTCCTCTCCCCGGGGTCCC | GGGACCCCGGGGAGAGGAGGGGTGGGGGG |
| CCCCCCACTCCCGACCCGACCCCAGCCCC | GGGGCTGGGGTCGGGTCGGGAGTGGGGGG |
| CCCCCCGCACCCCCGCTCCCACCCACCCC | GGGGTGGGTGGGAGCGGGGGTGCGGGGGG |
| CCCCCCGGTGCCCTTTCCCTTGTCAACCC | GGGTTGACAAGGGAAAGGGCACCGGGGGG |
| CCCCCCTCCCCCTTTCACCCCAAGCCCCC | GGGGGCTTGGGGTGAAAGGGGGAGGGGGG |
| CCCCCCTCCCGCGCCTCCCCGCAGGCCCC | GGGGCCTGCGGGGAGGCGCGGGACGGGGG |
| CCCCCCTGCCCTGGGGCCCCAGGCAGCCC | GGGCTGCCTGGGGCCCCAGGGCAGGGGGG |
| CCCCCGCCCCCGCCCGCCCGCTGCCCCCC | GGGGGGCAGCGGGCGGGCGGGGCGGGGG |
| CCCCCGCCCGCCGGCCCCCCGGCCCGCCC | GGGCGGGCCCGGGGGCCGGCGGGCGGGGG |
| CCCCCCCGTCCCTCCCCTCCCGCCTTCCC | GGGAAGGCGGGAGGGGAGGGACGCGGGGG |
| CCCCCGTCCCCTCCCCGACCCCGAGACCC | GGGTCTCGGGGTCGGGGAGGGGACGGGGG |
| CCCCCTCCCCGCGCGCCCCTCCCGCGCCC | GGGCGCGGGAGGGGCGCGCGGGGAGGGGG |
| CCCCCTCCTGCCCGGACGCCCTGGCACCC | GGGTGCCAGGGCGTCCGGGCAGGAGGGGG |
| CCCCCTGTCTCCCAGCCCCGGGCAGCCCC | GGGGCTGCCCGGGGCTGGGAGACAGGGGG |
| CCCCGCTCCTCCCAGCCCCATGCCACCCC | GGGGTGGCATGGGGCTGGGAGGAGCGGGG |
| CCCCGGCTCTCCCCTGCCCCACCTCACCC | GGGTGAGGTGGGGCAGGGGAGAGCCGGGG |
| CCCCTAACCCCCGATCCCCCGCCCGGCCC | GGGCCGGGCGGGGGATCGGGGGTTAGGGG |
| CCCCTACCTCCCGCCCCTACCCGTCCCCC | GGGGGACGGGTAGGGGCGGGAGGTAGGGG |
| CCCCTAGCTCCCCGGGCCCAGCTCGGCCC | GGGCCGAGCTGGGCCCGGGGAGCTAGGGG |
| CCCCTCCCCCACCAGCCCCTGCCCTGCCC | GGGCAGGGCAGGGGCTGGTGGGGGAGGGG |
| CCCCTCCCCTCCCTCCGCCCGCGCCCCCC | GGGGGGCGCGGGCGGAGGGAGGGGAGGGG |
| CCCCTCTCCCACCCCCACCCCCTACCCCC | GGGGGTAGGGGGTGGGGGTGGGAGAGGGG |
| CCCCTGCCAGCCCGCCCCCGGCCCGACCC | GGGTCGGGCCGGGGGCGGGCTGGCAGGGG |
| CCCCTGGGGCCCTGCCCCCACCTCCGCCC | GGGCGGAGGTGGGGGCAGGGCCCCAGGGG |
| CCCCTTCGGTCCCCAGCCCTGCCCTCCCC | GGGGAGGGCAGGGCTGGGGACCGAAGGGG |
| CCCGACACCCCTTCCTCCCTCCCCACCCC | GGGGTGGGGAGGGAGGAAGGGGTGTCGGG |
| CCCGACCCCACCCATGCCCAGACTGCCCC | GGGGCAGTCTGGGCATGGGTGGGGTCGGG |
| CCCGACTTCCCAGCGGCCCCGTGCGGCCC | GGGCCGCACGGGGCCGCTGGGAAGTCGGG |
| CCCGCACCCGGCAGCCCCCGCCCCCTCCC | GGGAGGGGGCGGGGGCTGCCGGGTGCGGG |
| CCCGCCCCACCCCACCCCCAATCTGCCCC | GGGGCAGATTGGGGGTGGGGTGGGGCGGG |
| CCCGCTCCCCCCGCCGCCTCCCCCTCCCC | GGGGAGGGGGAGGCGGCGGGGGGAGCGGG |
| CCCGGAAGCACCCTCCTCCCTAGGCCCCC | GGGGGCCTAGGGAGGAGGGTGCTTCCGGG |
| CCCGCCCCCAGCCCGGCCCGGCCCAGCCC | GGGCTGGGCCGGGCCGGGCTGGGGCCGGG |
| CCCGGGAAACCCCGTCGTTCCCTTTCCCC | GGGGAAAGGGAACGACGGGGTTTCCCGGG |
| CCCGGTACCCCGCGCCCCCACCCGCCCCC | GGGGGCGGGTGGGGGCGCGGGGTACCGGG |
| CCCGTCCCCCCTCGCCCCCGCCCCCTCCC | GGGAGGGGGCGGGGCCGAGGGGGGACGGG |
| CCCGTCCCCCTCGAGGCCCGGGCCCCCCC | GGGGGGGCCCGGGCCTCGAGGGGGACGGG |
| CCCGTGGTGCCCCTCCCCCGCCCCGCCCC | GGGGCGGGGCGGGGGAGGGGCACCACGGG |
| CCCTATCCCAGCCTCCCCCACAAGCCCCC | GGGGGCTTGTGGGGGAGGCTGGGATAGGG |
| CCCTCACCCCCCGCTCCCCCGCTCCCCCC | GGGGGGAGCGGGGGAGCGGGGGGTGAGGG |
| CCCTCAGAGCCCACACCCCACAGCGCCCC | GGGGCGCTGTGGGGTGTGGGCTCTGAGGG |
| CCCTCCAGGTCCCCTTCTTCCCCACTCCC | GGGAGTGGGGAAGAAGGGGACCTGGAGGG |
| CCCTCCCAGGCCCAGCCCCATCCCCACCC | GGGTGGGGATGGGGCTGGGCCTCGGAGGG |
| CCCTCCCCCACCCCAACCGCCCCCGCCCC | GGGGCGGGGGCGGTTGGGGTGGGGGAGGG |
| CCCTCCCCGCCCCTCCTGCCCCTCCTCCC | GGGAGGAGGGGCAGGAGGGGCGGGGAGGG |
| CCCTCCCCTGCCCCTCCCCTCCACCTCCC | GGGAGGTGGAGGGGAGGGGCAGGGGAGGG |
| CCCTCTCCCAGCCCTCCCCTACCCCACCC | GGGTGGGGTAGGGGAGGGCTGGGAGAGGG |
| CCCTGATGCCCAGCCTCCCTCCCCATCCC | GGGATGGGGAGGGAGGCTGGGCATCAGGG |
| CCCTGCGGTCCCGCCCCCTCCCCGCCCCC | GGGGGCGGGGAGGGGGCGGGACCGCAGGG |
| CCCTGGACCCCGCCGCCCCAACCTGGCCC | GGGCCAGGTTGGGGCGGCGGGGTCCAGGG |
| CCCTGTGCTCCCGGGACCCTCTGTCCCCC | GGGGGACAGAGGGTCCCGGGAGCACAGGG |
| CCCTTCTCTCCCCCTCCCCGCTCCCCCCC | GGGGGGGAGCGGGGAGGGGGAGAGAAGGG |
| CCCTTTCCTCCCCCACCCCTGGGGCTCCC | GGGAGCCCCAGGGGTGGGGGAGGAAAGGG |
| GGGAAAAGCGGGAAGGGAGGGAGGGAGGG | CCCTCCCTCCCTCCCTTCCCGCTTTTCCC |
| GGGAATAAAAGGGGACGGGGAGAGTGGGG | CCCCACTCTCCCCGTCCCCTTTTATTCCC |
| GGGACGGAGGGTGGGGGAGGGGGGGAGGG | CCCTCCCCCCCTCCCCCACCCTCCGTCCC |
| GGGAGAGAGGGAGAGAGGGAGGGCGGGGG | CCCCCGCCCTCCCTCTCTCCCTCTCTCCC |
| GGGAGAGGGAGGGAAGGGGGTTGGGCGGG | CCCGCCCAACCCCCTTCCCTCCCTCTCCC |
| GGGAGAGGGGCGCGGGAGGGAGGGAGGGG | CCCCTCCCTCCCTCCCGCGCCCCTCTCCC |
| GGGAGAGTGGGGCTTGGGGCCACATGGGG | CCCCATGTGGCCCCAAGCCCCACTCTCCC |
| GGGAGCTGGGACAGACTGGGGGGGCGGGG | CCCCGCCCCCCCAGTCTGTCCCAGCTCCC |
| GGGAGGAGGTGGGGTGGGGAGATGGCGGG | CCCGCCATCTCCCCACCCCACCTCCTCCC |
| GGGAGGCAGGGGCGAGGGGCTGCGGGGGG | CCCCCCGCAGCCCCTCGCCCCTGCCTCCC |
| GGGAGGCGTGGGTGGGGGGCTCCTTTGGG | CCCAAAGGAGCCCCCCACCCACGCCTCCC |
| GGGAGGGAAGGGGCCCGGGGACAGTTGGG | CCCAACTGTCCCCGGGCCCCTTCCCTCCC |
| GGGAGGGGGCGCTGGAGGGGGAGAGGGGG | CCCCCTCTCCCCCTCCAGCGCCCCCTCCC |
| GGGAGGGGGCGGGTCCTGAAGGGGGCGGG | CCCGCCCCCTTCAGGACCCGCCCCCTCCC |
| GGGAGGTCTTGGGGGTGTCCGGGAGGGGG | CCCCCTCCCGGACACCCCCAAGACCTCCC |
| GGGATTGGGCAGCTGGGGGGGGTGAAGGG | CCCTTCACCCCCCCCAGCTGCCCAATCCC |
| GGGCACAGGCGGGGGTGGGGGCGGGCGGG | CCCGCCCGCCCCCACCCCCGCCTGTGCCC |
| GGGCATCAGGGCCAGCAGGGAAGTAAGGG | CCCTTACTTCCCTGCTGGCCCTGATGCCC |
| GGGCCCCGGGAAGGGGCGGGGCGCGGGGG | CCCCCGCGCCCCGCCCCTTCCCGGGGCCC |
| GGGCCCGGTGGGTGGGGCTGGGGCTGGGG | CCCCAGCCCCAGCCCCACCCACCGGGCCC |
| GGGCCTCGCCGGGGTCGGGGGGCCCGGGG | CCCCGGGCCCCCCGACCCCGGCGAGGCCC |
| GGGCCTCGGGTTCAGGGGGCTGGGCAGGG | CCCTGCCCAGCCCCCTGAACCCGAGGCCC |
| GGGCCTGACCGGGCCGGGTCGGGGTCGGG | CCCGACCCCGACCCGGCCCGGTCAGGCCC |
| GGGCCTGGGAGGAGGGGGGCAGCGAAGGG | CCCTTCGCTGCCCCCCTCCTCCCAGGCCC |
| GGGCGCACAGGGCACTGCGGGTGGCGGGG | CCCCGCCACCCGCAGTGCCCTGTGCGCCC |
| GGGCGGGCGGGGCAGGGGGTGGGGCGGGG | CCCCGCCCCACCCCCTGCCCCGCCCGCCC |
| GGGCGGGGCCGGGAAGGGGAGGTATAGGG | CCCTATACCTCCCCTTCCCGGCCCCGCCC |
| GGGCTGACTGGGCCTCCAGGGTCGCAGGG | CCCTGCGACCCTGGAGGCCCAGTCAGCCC |
| GGGCTGAGGGATGAGGTGGGATTTCTGGG | CCCAGAAATCCCACCTCATCCCTCAGCCC |
| GGGCTGGTGGGAGGACTGGGCTGTGGGGG | CCCCCACAGCCCAGTCCTCCCACCAGCCC |
| GGGGAAAGGAGGGGTGGGGAGGGGGTGGG | CCCACCCCCTCCCCACCCCTCCTTTCCCC |
| GGGGACTCAGGGGACGGGGTGAGGAAGGG | CCCTTCCTCACCCCGTCCCCTGAGTCCCC |
| GGGGACTGAAGGGCAGGGGAGGGCAAGGG | CCCTTGCCCTCCCCTGCCCTTCAGTCCCC |
| GGGGAGGAGGGTGGGGAAGGGGAGAGGGG | CCCCTCTCCCCTTCCCCACCCTCCTCCCC |
| GGGGAGTGGGAGGTGGGGGTGCGTGGGGG | CCCCCACGCACCCCCACCTCCCACTCCCC |
| GGGGAGTGGGGGGCGAGGCGGGGCCAGGG | CCCTGGCCCCGCCTCGCCCCCCACTCCCC |
| GGGGATGGGGGGGATGGGAGGGGACAGGG | CCCTGTCCCCTCCCATCCCCCCCATCCCC |
| GGGGCAGAGGGGAGGGCAGGGGAGCAGGG | CCCTGCTCCCCTGCCCTCCCCTCTGCCCC |
| GGGGCCCATCGGGGGAGGGGCGGAGGGGG | CCCCCTCCGCCCCTCCCCCGATGGGCCCC |
| GGGGCCGCCAGGGAAGTGTGGGGGGCGGG | CCCGCCCCCCACACTTCCCTGGCGGCCCC |
| GGGGCCGTGGGGGTAGGGGCAGGGCTGGG | CCCAGCCCTGCCCCTACCCCCACGGCCCC |
| GGGGCGGCGTGGGTGGGGGCGGGGGCGGG | CCCGCCCCCGCCCCCACCCACGCCGCCCC |
| GGGGCGTGGGGCGGGTGGGCCACAGGGGG | CCCCCTGTGGCCCACCCGCCCCACGCCCC |
| GGGGCTCCGGGACACGGGGTCAACGGGGG | CCCCCGTTGACCCCGTGTCCCGGAGCCCC |
| GGGGCTGGGGCGCGGGTGGGCGGTCGGGG | CCCCGACCGCCCACCCGCGCCCCAGCCCC |
| GGGGGAACTGGGAGGGGCTGGGACCGGGG | CCCCGGTCCCAGCCCCTCCCAGTTCCCCC |
| GGGGGAGCGGGGAGGCGGGGTGCTCTGGG | CCCAGAGCACCCCGCCTCCCCGCTCCCCC |
| GGGGGAGGGGGAAGAGGGGCGGAGTTGGG | CCCAACTCCGCCCCTCTTCCCCCTCCCCC |
| GGGGGAGTGGGGCTGCGAGGGGGAGGGGG | CCCCCTCCCCCTCGCAGCCCCACTCCCCC |
| GGGGGCGCGCGGGGCAGAGGGGGAGCGGG | CCCGCTCCCCCTCTGCCCCGCGCGCCCCC |
| GGGGGGAGGGTGGGCAGGGATACTCAGGG | CCCTGAGTATCCCTGCCCACCCTCCCCCC |
| GGGGGGCGCGGGGTCAGGGACCCGGTGGG | CCCACCGGGTCCCTGACCCCGCGCCCCCC |
| GGGGGGGAGCGGGCTGGGGCGGGGCAGGG | CCCTGCCCCGCCCCAGCCCGCTCCCCCCC |
| GGGGGGGGCAGGGGCGGGGCAGGGGCGGG | CCCGCCCCTGCCCCGCCCCTGCCCCCCCC |
| GGGGGGTGCGGGAGGGGGTGGGGGCAGGG | CCCTGCCCCCACCCCCTCCCGCACCCCCC |
| GGGGGTTAGGGGCCGAGGGCTGTGGGGGG | CCCCCCACAGCCCTCGGCCCCTAACCCCC |
| GGGGTAGGGGCAGGAGGGGACGGGGTGGG | CCCACCCCGTCCCCTCCTGCCCCTACCCC |
| GGGGTCGAGGGGAGAGTGGGGGTGGCGGG | CCCGCCACCCCCACTCTCCCCTCGACCCC |
| GGGGTCTGGGAGATACTGGGAGGGAGGGG | CCCCTCCCTCCCAGTATCTCCCAGACCCC |
| GGGGTGAGGAGGGGAAAGGGGAAACGGGG | CCCCGTTTCCCCTTTCCCCTCCTCACCCC |
| GGGGTGCGGGGGGCGGGGGCAGTACCGGG | CCCGGTACTGCCCCCGCCCCCCGCACCCC |
| GGGGTGGGGCAGGGCAGGGTTGGGTAGGG | CCCTACCCAACCCTGCCCTGCCCCACCCC |
| GGGGTTGGGGCGGGGCGGGGCGGGCGGGG | CCCCGCCCGCCCCGCCCCGCCCCAACCCC |
| GGGTCCTGAGGGGTGAGCAGGGGTTGGGG | CCCCAACCCCTGCTCACCCCTCAGGACCC |
| GGGTCTCAGGGATATGGGGGTTCTTGGGG | CCCCAAGAACCCCCATATCCCTGAGACCC |
| GGGTGGCGTGGGGGGCAGGGGTGGGTGGG | CCCACCCACCCCTGCCCCCCACGCCACCC |
| GGGTTCGGGGACGCCCGGGCCGGGCAGGG | CCCTGCCCGGCCCGGGCGTCCCCGAACCC |
| GGGTTTGGGGTGGGGAAGGGAAGGGCGGG | CCCGCCCTTCCCTTCCCCACCCCAAACCC |
| CCCACAGGGTCCCCAGCCCCACCCCAGCCC | GGGCTGGGGTGGGGCTGGGGACCCTGTGGG |
| CCCACCGGCCCCCGACGCCCTCACTGCCCC | GGGGCAGTGAGGGCGTCGGGGGCCGGTGGG |
| CCCACTGCAGCCCCTGGCGCCCCCTACCCC | GGGGTAGGGGGCGCCAGGGGCTGCAGTGGG |
| CCCAGCAGCCCCTGCAGCCCCTGTAGCCCC | GGGGCTACAGGGGCTGCAGGGGCTGCTGGG |
| CCCAGCTGCCCAGCCCTGCCCCCCCTCCCC | GGGGAGGGGGGGCAGGGCTGGGCAGCTGGG |
| CCCCACACCTCCCTCCGGCTCCCTCCTCCC | GGGAGGAGGGAGCCGGAGGGAGGTGTGGGG |
| CCCCCAAACCCGCACCTCCCGGGCGCCCCC | GGGGGCGCCCGGGAGGTGCGGGTTTGGGGG |
| CCCCCAACCCCTGAGCCCCCAGCCGAGCCC | GGGCTCGGCTGGGGGCTCAGGGGTTGGGGG |
| CCCCCACCCCGCGGCAGCCCCGCCCAGCCC | GGGCTGGGCGGGGCTGCCGCGGGGTGGGGG |
| CCCCCCACCCGCCCCATCCCCCGGACTCCC | GGGAGTCCGGGGGATGGGGCGGGTGGGGGG |
| CCCCCCTAGCCCCGGGAGCCCCCAGCGCCC | GGGCGCTGGGGGCTCCCGGGGCTAGGGGGG |
| CCCCCGACCCCAAGCCCTCCCTCAGGGCCC | GGGCCCTGAGGGAGGGCTTGGGGTCGGGGG |
| CCCCCGCCCCCCACCCCACCCTCCCTTCCC | GGGAAGGGAGGGTGGGGTGGGGGGCGGGGG |
| CCCCCGCCCCGCGCCGCCCCGCCCCGCCCC | GGGGCGGGGCGGGGCGGCGCGGGGCGGGGG |
| CCCCCTCCCCCTCCCTCCCCCCGCTCCCCC | GGGGGAGCGGGGGGAGGGAGGGGGAGGGGG |
| CCCCCTCTCTCCCGCCATTCCCAGATGCCC | GGGCATCTGGGAATGGCGGGAGAGAGGGGG |
| CCCCGACCTCCCTCGCGCCCTGTCCCCCCC | GGGGGGCACAGGGCGCGGAGGGAGGTCGGGG |
| CCCCGCCCGCCCTCCTCTGCCCCCTCCCCC | GGGGGAGGGGGCAGAGGAGGGCGGGCGGGG |
| CCCCGCTCCTCCCCCAACCCGACCCCACCC | GGGTGGGGTCGGGTTGGGGGAGGAGCGGGG |
| CCCCGGGGCCCCGGGCCCCCACCCGAGCCC | GGGCTCGGGTGGGGGCCCGGGGGCCCCGGG |
| CCCCGGGTCCCACCCGGCCCGGCCCGGCCC | GGGCCGGGCCGGGCCGGGTGGGACCCGGGG |
| CCCCGGTCTCCCCTTACCACCCCCACCCCC | GGGGGTGGGGGTGGTAAGGGGAGACCGGGG |
| CCCCTCCCCCAGTCCCCCCCCCCATCCCCC | GGGGGATGGGGGGGGGGACTGGGGGAGGGG |
| CCCCTCCTGACCCCATCCCCTCCCTGCCCC | GGGGCAGGGAGGGGATGGGGTCAGGAGGGG |
| CCCCTCGCCCCGCCTGCCCCTTTGCACCCC | GGGGTGCAAAGGGGCAGGCGGGGCGAGGGG |
| CCCCTGTCCCCTTCACGCCCAGCCCCACCC | GGGTGGGGCTGGGCGTGAAGGGGACAGGGG |
| CCCCTTCTTCCCATCCCACCCGCCCCCCCC | GGGGGGGGCGGGTGGGATGGGAAGAAGGGG |
| CCCCTTGCCCTCAGTTCCCCACTGGCCCCC | GGGGGCCAGTGGGGAACTGAGGGCAAGGGG |
| CCCGACCAGGCCCCAGCCCCTGCCCAGCCC | GGGCTGGGCAGGGGCTGGGGCCTGGTCGGG |
| CCCGACCCGTCCCCCTGCCCTCTGCCCCCC | GGGGGGCAGAGGGCAGGGGGACGGGTCGGG |
| CCCGCCGCCCGCCTGCGCCCCCGCGCGCCC | GGGCGCGCGGGGGCGCAGGCGGGCGGCGGG |
| CCCGCCGGCCCCGCCCCCGCCCCGCCCCCC | GGGGGGCGGGGCGGGGGCGGGGCCGGCGGG |
| CCCGCGCTCTCCCTCCTCCCTGCCTCCCCC | GGGGGAGGCAGGGAGGAGGGAGAGCGCGGG |
| CCCGGAATATCCCAAATTCCCGTGCAGCCC | GGGCTGCACGGGAATTTGGGATATTCCGGG |
| CCCGGCAGCCCATCCTTCCCAGGGGGGCCC | GGGCCCCCCTGGGAAGGATGGGCTGCCGGG |
| CCCGGCCCGGCCCGGCCCGGCCCGGCCCCC | GGGGGCCGGGCCGGGCCGGGCCGGGCCGGG |
| CCCGGCCGGTCCCCCCACCCGTCCCTTCCC | GGGAAGGGACGGGTGGGGGGACCGGCCGGG |
| CCCGGCTGCCCAGCGTGCCCCTCGACTCCC | GGGAGTCGAGGGGCACGCTGGGCAGCCGGG |
| CCCGGGAGCCCAGAGGTGCCCCCCCAACCC | GGGTTGGGGGGGCACCTCTGGGCTCCCGGG |
| CCCGGGCACTCCCGGGTCCCTCACAGCCCC | GGGGCTGTGAGGGACCCGGGAGTGCCCGGG |
| CCCGTATTCCCCCAGACCCCAGAGAGCCCC | GGGGCTCTCTGGGGTCTGGGGGAATACGGG |
| CCCTCAGCCCACCAGAGCCCCACCAGGCCC | GGGCCTGGTGGGGCTCTGGTGGGCTGAGGG |
| CCCTCATCCCCACCCCACCCAGCCCACCCC | GGGGTGGGCTGGGTGGGGTGGGGATGAGGG |
| CCCTCCCACCCCCCACCCCTCCCCACCCCC | GGGGGTGGGGAGGGGTGGGGGGTGGGAGGG |
| CCCTCCGATGCCCTTTCCCACCCGCCGCCC | GGGCGGCGGGTGGGAAAGGGCATCGGAGGG |
| CCCTCCGCCCCCACCCCGACCCGCACCCCC | GGGGGTGCGGGTCGGGGTGGGGGCGGAGGG |
| CCCTCCTCCCCCCGCCTCCCTTCTCTTCCC | GGGAAGAGAAGGGAGGCGGGGGGAGGAGGG |
| CCCTCCTCCCTCACCACCCCACCCCACCCC | GGGGTGGGGTGGGGTGGTGAGGGAGGAGGG |
| CCCTCCTCCCTGCTCGCCCCCAGATTCCCC | GGGGAATCTGGGGGCGAGCAGGGAGGAGGG |
| CCCTCCTTCCCAGGGCCGCCCCCGCCCCCC | GGGGGGCGGGGGCGGCCCTGGGAAGGAGGG |
| CCCTCGCCCCCGGGCTCGCCCTTGGCCCCC | GGGGGCCAAGGGCGAGCCCGGGGGCGAGGG |
| CCCTCGCTCTCCCACCCAGCCCCCTCCCCC | GGGGGAGGGGGCTGGGTGGGAGAGCGAGGG |
| CCCTCTTCCCCCCAACCCCCCCTCAGCCCC | GGGGCTGAGGGGGGGTTGGGGGGAAGAGGG |
| CCCTCTTCTGCCCCTGCCCCTACTGCCCCC | GGGGGCAGTAGGGGCAGGGGCAGAAGAGGG |
| CCCTGGACCTCCCGGACCCCCCGGTGTCCC | GGGACACCGGGGGGTCCGGGAGGTCCAGGG |
| CCCTGGCCTGCCCTGACCCCTGCCCCTCCC | GGGAGGGGCAGGGGTCAGGGCAGGCCAGGG |
| CCCTGTGCTGCCCTGCCCCCAGCTCCACCC | GGGTGGAGCTGGGGGCAGGGCAGCACAGGG |
| CCCTGTTGAGCCCAGGCCCCCTCCCACCCC | GGGGTGGGAGGGGGCCTGGGCTCAACAGGG |
| CCCTTCACCCCACCCCCACCCCCACCCCCC | GGGGGGTGGGGGTGGGGGTGGGGTGAAGGG |
| CCCTTCTCTGCCCTTTCTGCCCTCCTTCCC | GGGAAGGAGGGCAGAAAGGGCAGAGAAGGG |
| GGGAACAGGGGTGGGGGCGGGAGGTGAGGG | CCCTCACCTCCCGCCCCCACCCCTGTTCCC |
| GGGACGGGCTGGGGTGCAGGGGGTTCTGGG | CCCAGAACCCCCTGCACCCCAGCCCGTCCC |
| GGGAGAGCTGGGGAGCCCTAGGGGAGCGGG | CCCGCTCCCCTAGGGCTCCCCAGCTCTCCC |
| GGGAGAGGAGGGCTGGGGGGGTAGTCAGGG | CCCTGACTACCCCCCCAGCCCTCCTCTCCC |
| GGGAGGAGCTGGGCACTGGGCCACAGGGGG | CCCCCTGTGGCCCAGTGCCCAGCTCCTCCC |
| GGGAGGGGAGGGGGAAGCGAGGGGCGTGGG | CCCACGCCCCTCGCTTCCCCCTCCCCTCCC |
| GGGAGGGGTGGGGGCGGGGGCGGGGTGGGG | CCCCACCCCGCCCCCGCCCCCACCCCTCCC |
| GGGAGTGAGGGCAGGGGCGGGATCCAAGGG | CCCTTGGATCCCGCCCCTGCCCTCACTCCC |
| GGGATTCAGTGGGTGAGAGGGAAGAAGGGG | CCCCTTCTTCCCTCTCACCCACTGAATCCC |
| GGGATTTATGGGGACAGGGGAAGGTCAGGG | CCCTGACCTTCCCCTGTCCCCATAAATCCC |
| GGGCCGGGCCGGGCCGGGGGTGGGCGGGGG | CCCCCGCCCACCCCCGGCCCGGCCCGGCCC |
| GGGCCGGGGCGGGGAGATGGGTGGGAAGGG | CCCTTCCCACCCATCTCCCCGCCCCGGCCC |
| GGGCCGGGGTGGGGGGTTGGGGGACGGGGG | CCCCCGTCCCCCAACCCCCCACCCCGGCCC |
| GGGCCTGGAGGGGGTGGGGGACCCGGCGGG | CCCGCCGGGTCCCCCACCCCCTCCAGGCCC |
| GGGCGAACGGGCGTCCGGGGACAGGGTGGG | CCCACCCTGTCCCCGGACGCCCGTTCGCCC |
| GGGCGCGGGGGCGCGCGGGGTCCTGGCGGG | CCCGCCAGGACCCCGCGCGCCCCCGCGCCC |
| GGGCGGGGGAGGGGGGAGTTGGGGGGAGGG | CCCTCCCCCCAACTCCCCCCTCCCCCGCCC |
| GGGCGTTGGGGGCTCGCGGGGGCGTGGGGG | CCCCCACGCCCCCGCGAGCCCCCAACGCCC |
| GGGCTGCGGGGAGCGGAGGGAGGGGCGGGG | CCCCGCCCCTCCCTCCGCTCCCCGCAGCCC |
| GGGCTTTCCTGGGAGTGGGTGGGGAGGGGG | CCCCCTCCCCACCCACTCCCAGGAAAGCCC |
| GGGGAAGAGAGGGGGAGGGGAGCTCAAGGG | CCCTTGAGCTCCCCTCCCCCTCTCTTCCCC |
| GGGGAAGTGGGACCTTCGGGATTGTGGGGG | CCCCCACAATCCCGAAGGTCCCACTTCCCC |
| GGGGACGCTGGGCACCTGGGGGCGCTAGGG | CCCTAGCGCCCCCAGGTGCCCAGCGTCCCC |
| GGGGACTGAGGGCTTTTGGGACCCTGCGGG | CCCGCAGGGTCCCAAAAGCCCTCAGTCCCC |
| GGGGACTGGGTGGGGAGGGGCGGGGAGGGG | CCCCTCCCCGCCCCTCCCCACCCAGTCCCC |
| GGGGAGAGCTGGGGGCCAGGGGAGAAGGGG | CCCCTTCTCCCCTGGCCCCCAGCTCTCCCC |
| GGGGAGAGGGGAGGGGAGGGGGGGAGGGGG | CCCCCTCCCCCCCTCCCCTCCCCTCTCCCC |
| GGGGAGAGGGGGCTGGGTGGGCAGCAAGGG | CCCTTGCTGCCCACCCAGCCCCCTCTCCCC |
| GGGGAGGAGTGGGGGAGGGGTGGGGGCGGG | CCCGCCCCCACCCCTCCCCCACTCCTCCCC |
| GGGGCCGAGCGGGAGGGCACGGGCGGGGGG | CCCCCCGCCCGTGCCCTCCCGCTCGGCCCC |
| GGGGCCGGAGGGACCGAGGGGGAGGGCGGG | CCCGCCCTCCCCCTCGGTCCCTCCGGCCCC |
| GGGGCGGCGGGGAGAAGTAGGGGCGAGGGG | CCCCTCGCCCCTACTTCTCCCCGCCGCCCC |
| GGGGCGGGCGGGGCAGCTGGGGAGGGAGGG | CCCTCCCTCCCCAGCTGCCCCGCCCGCCCC |
| GGGGCTGCGCGGGGCTGGGGGGCTGCTGGG | CCCAGCAGCCCCCCAGCCCCGCGCAGCCCC |
| GGGGCTGGGGCAGGAGTGGGAAGGGCTGGG | CCCAGCCCTTCCCACTCCTGCCCCAGCCCC |
| GGGGCTTGGGGTGTGGGAGGGACCAAGGGG | CCCCTTGGTCCCTCCCACACCCCAAGCCCC |
| GGGGGAGCGGGCGGGCGGGGGCAGTTGGGG | CCCCACTGCCCCCGCCCGCCCGCTCCCCC |
| GGGGGAGGGTGGGAAAGTTTGGGGGGGGGG | CCCCCCCCCCAAACTTTCCCACCCTCCCCC |
| GGGGGATCTGGGGGCATGGGTTCGGGAGGG | CCCTCCCGAACCCATGCCCCCAGATCCCCC |
| GGGGGCGGGGTGGCGGCGGGAGGGGCGGGG | CCCCGCCCCTCCCGCCGCCACCCCGCCCCC |
| GGGGGGCAGGGGCGGGCAGGGATTAAAGGG | CCCTTTAATCCCTGCCCGCCCCTGCCCCCC |
| GGGGGGCTCTGGGTGCCCAGGGGAGCCGGG | CCCGGCTCCCCTGGGCACCCAGAGCCCCCC |
| GGGGGGGTGGGGGGTGGGGTGGGGGGAGGG | CCCTCCCCCCACCCCACCCCCCACCCCCCC |
| GGGGGTGGGGAGAGGTAGGGACAGGAAGGG | CCCTTCCTGTCCCTACCTCTCCCCACCCCC |
| GGGGTCCAGGGGAGACGGGGGTCGGCGGGG | CCCCGCCGACCCCCGTCTCCCCTGGACCCC |
| GGGGTGCAGAGGGAAGCTGGGGCCTTGGGG | CCCCAAGGCCCCAGCTTCCCTCTGCACCCC |
| GGGGTGGGAGGGAGGGCCCGGGGGGCGGGG | CCCCGCCCCCCGGGCCCTCCCTCCCACCCC |
| GGGGTTGGGGGTGGGTGGGGAGCTTTTGGG | CCCAAAAGCTCCCCACCCACCCCCAACCCC |
| GGGTCCGGGGCAGGGCTGGGGATCTGGGGG | CCCCCAGATCCCCAGCCCTGCCCCGGACCC |
| GGGTCCTAGGGTACAGAGGGCGTTTGGGGG | CCCCCAAACGCCCTCTGTACCCTAGGACCC |
| GGGTGGAATGGGCGGCCTGGGGTTACTGGG | CCCAGTAACCCCAGGCCGCCCATTCCACCC |
| GGGTGGGAATGGGGTGGAAGGGTGATGGGG | CCCCATCACCCTTCCACCCCATTCCCACCC |
| GGGTGGTGGTGGGTGAGTGGGTGGGTGGGG | CCCCACCCACCCACTCACCCACCACCACCC |
| GGGTTTGAATGGGGACAGGGTGCGAGAGGG | CCCTCTCGCACCCTGTCCCCATTCAAACCC |
| CCCACCGTGGCCCCTACGCCCAATTAACCCC | GGGGTTAATTGGGCGTAGGGGCCACGGTGGG |
| CCCACGGCAGCCCTCAGGGCCCGCTGGCCCC | GGGGCCAGCGGGCCCTGAGGGCTGCCGTGGG |
| CCCAGACCCGCCCCCACTTCCCCCCTCACCC | GGGTGAGGGGGGAAGTGGGGGCGGGTCTGGG |
| CCCAGATCCCCCTCCCCCACCCCCTGTGCCC | GGGCACAGGGGGTGGGGGAGGGGGATCTGGG |
| CCCAGCCGCGCCCCCTCCCTCCCTGGTGCCC | GGGCACCAGGGAGGGAGGGGGCGCGGCTGGG |
| CCCATCTGCCCCGCCCAGCCCTGCCCTGCCC | GGGCAGGGCAGGGCTGGGCGGGGCAGATGGG |
| CCCATTCCTCCCACCTTTCCCTCCCCTTCCC | GGGAAGGGGAGGGAAAGGTGGGAGGAATGGG |
| CCCCACTGCCCTCCCCCGCCCCCCAGCGCCC | GGGCGCTGGGGGGCGGGGGAGGGCAGTGGGG |
| CCCCATCCCGCCCCACCCCACCCTCATTCCC | GGGAATGAGGGTGGGGTGGGGCGGGCATGGGG |
| CCCCATGATGCCCCCGGCCCCCCGGCCCCC | GGGGGCCGGGGGGCCCGGGGGCATCATGGGG |
| CCCCCAGCTCCCCCTCAGCCCTGCCCTACCC | GGGTAGGGCAGGGCTGAGGGGGAGCTGGGGG |
| CCCCCATCCCCTCCAGCCCCCAAGGCTGCCC | GGGCAGCCTTGGGGGCTGGAGGGGATGGGGG |
| CCCCCCACTTCCCTCACCCTCCCTATACCCC | GGGGTATAGGGAGGGTGAGGGAAGTGGGGGG |
| CCCCGGCTGCCCCCCGCCCCCAGGCTGCCCC | GGGGCAGCCTGGGGGCGGGGGGCAGCCGGGG |
| CCCCGGGACGCCCTGCAGACCCACGAGCCCC | GGGGCTCGTGGGTCTGCAGGGCGTCCCGGGG |
| CCCCGGGGCGCCCGGCGGGCCCCGCCCCC | GGGGGCGGGGGGCCCGCCGGGCGCCCCGGGG |
| CCCCTCCAACCCCTCTCTGCCCTGGAAGCCC | GGGCTTCCAGGGCAGAGAGGGGTTGGAGGGG |
| CCCCTCCCACCCCTCTCTGCCCTGGGAGCCC | GGGCTCCCAGGGCAGAGAGGGGTGGGAGGGG |
| CCCCTCCCTTCCCACCCGCACCCTGCCGCCC | GGGCGGAGGGTGCGGGTGGGAAGGGAGGGG |
| CCCGGCATTCCCCGCGCTCCCCGAGCTTCCC | GGGAAGCTCGGGGAGCGCGGGGAATGCCGGG |
| CCCGGCGGTCCCGGGGGACCCGGGGGGCCCC | GGGGCCCCCCGGGTCCCCCGGGACCGCCGGG |
| CCCGGGAAATCCCGCCCACTCCCCCGACCCC | GGGGTCGGGGGAGTGGGCGGGATTTCCCGGG |
| CCCGGGCGTCCCCGCAGACCCCGGCAGGCCC | GGGCCTGCCGGGGTCTGCGGGGACGCCCGGG |
| CCCGTAGTCCCCTACGCCCCCCGGGGCCCCC | GGGGGCCCCGGGGGGCGTAGGGGACTACGGG |
| CCCTCAGGCTCCCAGGCCCCCCGCCGCCCCC | GGGGGCGGCGGGGGGCCTGGGAGCCTGAGGG |
| CCCTCCATCCCCATCAGCCCCGAAGATCCCC | GGGGATCTTCGGGGCTGATGGGGATGGAGGG |
| CCCTCCCTGGCCCCCCAACCCTCTTCCTCCC | GGGAGGAAGAGGGTTGGGGGGCCAGGGAGGG |
| CCCTCCTTCTCCCCGCCGTCCCCACACCCCC | GGGGGTGTGGGGACGGCGGGGAGAAGGAGGG |
| CCCTCTCCTCCCCCAGTCCACCCTGCACCCC | GGGGTGCAGGGTGGACTGGGGGAGGAGAGGG |
| CCCTGACATTCCCCTCCTCCCACCCCGCCCC | GGGGCGGGGTGGGAGGAGGGGAATCTCAGGG |
| CCCTGCCCCGCCCCTCCTCCCGCCTCCACCC | GGGTGGAGGCGGGAGGAGGGGCGGGGCAGGG |
| CCCTGGTGCCCTCTGCCACCCCACGGCACCC | GGGTGCCGTGGGGTGGCAGAGGGCACCAGGG |
| CCCTGTCCACCCCCGCCTCCCTCCCCTGCCC | GGGCAGGGGAGGGAGGCGGGGGTGGACAGGG |
| CCCTTCACGGCCCCGATTCCCGGCCCCTCCC | GGGAGGGGCCGGGAATCGGGGCCGTGAAGGG |
| CCCTTTGTTCCCCCTCCCCCCCGCGGCACCC | GGGTGCCGCGGGGGGGAGGGGGAACAAAGGG |
| GGGAAAGGGCGGGCGGCCGGGCTGGCTGGGG | CCCCAGCCAGCCCGGCCGCCCGCCCTTTCCC |
| GGGAAGAGGCGGGGGAAGGGGGAGTCAGGGG | CCCCTGACTCCCCCTTCCCCCGCCTCTTCCC |
| GGGACTGCAGGGCTCTGGGGGCCGGGGCGGG | CCCGCCCCGGCCCCCAGAGCCCTGCAGTCCC |
| GGGAGAGCACGGGACCCGGTGGGGGAGGGGG | CCCCCTCCCCCACCGGGTCCCGTGCTCTCCC |
| GGGAGGCGCAGGGACATGGGGCAAGCCAGGG | CCCTGGCTTGCCCCATGTCCCTGCGCCTCCC |
| GGGAGGGGGAGGGCGTCGGGGGGGTGGGGGG | CCCCCCACCCCCCCGACGCCCTCCCCCTCCC |
| GGGAGGGGTGGGGTGGCCGGGAGGGCAGGGG | CCCCTGCCCTCCCGGCCACCCCACCCCTCCC |
| GGGCACCTGGGGGACGCGCGGGGCACTCGGG | CCCGAGTGCCCCGCGCGTCCCCCAGGTGCCC |
| GGGCAGAAGGGCTCCATGGGGGCCCCTGGGG | CCCCAGGGGCCCCCATGGAGCCCTTCTGCCC |
| GGGCAGATGGGGGCAGTGACGGGGTATGGGG | CCCCATACCCCGTCACTGCCCCCATCTGCCC |
| GGGCAGGGCGGGCGGGCAGGGTAAGGTGGGG | CCCCACCTTACCCTGCCCGCCCGCCCTGCCC |
| GGGCCCCCCCGGGGGCCGGGGCGGGCCGGGG | CCCCGGCCCGCCCCGGCCCCCGGGGGGGCCC |
| GGGCCCCGCCGGGAACTGGGGCCCCCCTGGG | CCCAGGGGGGCCCCAGTTCCCGGCGGGGCCC |
| GGGCCGGCGAGGGGGCGCGGGCGGGCGGGGG | CCCCCGCCCGCCCGCGCCCCCTCGCCGGCCC |
| GGGCGTCAGGGGCGTCAGGGGCAGCTGCGGG | CCCGCAGCTGCCCCTGACGCCCCTGACGCCC |
| GGGCTCCCAGGGCAGAGAGGGGTGGGAGGGG | CCCCTCCCACCCCTCTCTGCCCTGGGAGCCC |
| GGGCTGTTTGGGGTGAGAGTGGGGGTGGGGG | CCCCCACCCCCACTCTCACCCCAAACAGCCC |
| GGGGAAGGGGGTGGGCTGGGGAGCTGCTGGG | CCCAGCAGCTCCCCAGCCCACCCCCTTCCCC |
| GGGGACCTCTGGGTCAGCTGGGAGAGCGGGG | CCCCGCTCTCCCAGCTGACCCAGAGGTCCCC |
| GGGGAGACTCGGGTTAGGAAGGGGGCCCGGG | CCCGGGCCCCCTTCCTAACCCGAGTCTCCCC |
| GGGGCAGAGGGGGACTTGGGGTTGGGCAGGG | CCCTGCCCAACCCCAAGTCCCCCTCTGCCCC |
| GGGGCCCGGCGGGGGCTGGGGAGGGGCCGGG | CCCGGCCCCTCCCCAGCCCCCGCCGGGCCCC |
| GGGGCGGGGAGGGAGCCCGGGAGGCACCGGG | CCCGGTGCCTCCCGGGCTCCCTCCCCGCCCC |
| GGGGCTGGGGGCGGGCGCGGGGGGCGCAGGG | CCCTGCGCCCCCCGCGCCCGCCCCCAGCCCC |
| GGGGGAGGTGGGTGGAAGGGGTGAGTGCGGG | CCCGCACTCACCCCTTCCACCCACCTCCCCC |
| GGGGGCAGAGGGAAAGTAGGGTGGTACAGGG | CCCTGTACCACCCTACTTTCCCTCTGCCCCC |
| GGGGGCGGGGGCGGCCCCGGGGAGGGCGGGG | CCCCGCCCTCCCCGGGGCCGCCCCCGCCCCC |
| GGGGGCGGGGGGACCGGAGGGCAGGAGCGGG | CCCGCTCCTGCCCTCCGGTCCCCCCGCCCCC |
| GGGGGCTTGGGACGCGAGGGGGGGGGGAGGG | CCCTCCCCCCCCCCTCGCGTCCCAAGCCCCC |
| GGGGGGTACTGGGCGGGGGGGTAGGGCTGGG | CCCAGCCCTACCCCCCCGCCCAGTACCCCCC |
| GGGGGGTGGGGAGGGGGCGGGTGGCAATGGG | CCCATTGCCACCCGCCCCCTCCCCACCCCCC |
| GGGGTACTAGGGTGGGTGGGGGCCTGAGGGG | CCCCTCAGGCCCCCACCCACCCTAGTACCCC |
| GGGGTGGCAGGGGCGGGGCCGGGGGCGGGGG | CCCCCGCCCCCGGCCCCGCCCCTGCCACCCC |
| GGGGTTGGGGGCTGTGGCGGGTGGATTGGGG | CCCCAATCCACCCGCCACAGCCCCCAACCCC |
| GGGTCAGCCTGGGCCAGTGGGCCCCCAGGGG | CCCCTGGGGGCCCACTGGCCCAGGCTGACCC |
| GGGTGGGGGCGGGGGCGGGGGCAGGTCCGGG | CCCGGACCTGCCCCCGCCCCCGCCCCCACCC |
| GGGTGTGGAAGGGTGGGTTTGGGGCCGGGGG | CCCCCGGCCCCAAACCCACCCTTCCACACCC |
| CCCACTCATGCCCACCCACCCCGAGGGGCCCC | GGGGCCCCTCGGGGTGGGTGGGCATGAGTGGG |
| CCCACTTTACCCACCCCTGCCCCACCCTACCC | GGGTAGGGTGGGGCAGGGGTGGGTAAAGTGGG |
| CCCAGCCCAGCCCACTCTGCCCTTAGAGGCCC | GGGCCTCTAAGGGCAGAGTGGGCTGGGCTGGG |
| CCCAGGGCCTCCCCGCAGTCCCTGCCTAGCCC | GGGCTAGGCAGGGACTGCGGGGAGGCCCTGGG |
| CCCAGGTGCACCCGCAGAGCCCACTGTGTCCC | GGGACACAGTGGGCTCTGCGGGTGCACCTGGG |
| CCCCACCCCCCCACCGGCGCCCGCCCTCGCCC | GGGCGAGGGCGGGCGCCGGTGGGGGGGTGGGG |
| CCCCACCTGTCCCTGCCCATCCCTTGGTCCCC | GGGGACCAAGGGATGGGCAGGGACAGGTGGGG |
| CCCCCAAGTCCCCAGTCTGGCCCACCTTCCCC | GGGGAAGGTGGGCCAGACTGGGGACTTGGGGG |
| CCCCCAGTCGCCCCCGGGATCCCCCCCAACCC | GGGTTGGGGGGGATCCCGGGGGCGACTGGGGG |
| CCCCCGCTCCCCAACCCATCCCTTCCCAGCCC | GGGCTGGGAAGGGATGGGTTGGGGAGCGGGGG |
| CCCCCTCCCCCCAGCTCCTCCCTACTACCCCC | GGGGGTAGTAGGGAGGAGCTGGGGGGAGGGGG |
| CCCCGCCCCGCCCCCTTTCCCCACCGCCACCC | GGGTGGCGGTGGGGAAAGGGGGCGGGGCGGGG |
| CCCCGCCTCCCCACTCTGCCCCCGCCTACCCC | GGGGTAGGCGGGGGCAGAGTGGGGAGGCGGGG |
| CCCCGCGTGACCCCCCCTTCCCTTCCCTTCCC | GGGAAGGGAAGGGAAGGGGGGGTCACGCGGGG |
| CCCCGGCGCCCCGCCGACTCCCGCTCCCGCCC | GGGCGGGAGCGGGAGTCGGCGGGGCGCCGGGG |
| CCCCTCCGCTCCCCGGGCCTCCCACTGCGCCC | GGGCGCAGTGGGAGGCCCGGGGAGCGGAGGGG |
| CCCCTGTGCCCCGCCCTCTCCCTCGCCCTCCC | GGGAGGGCGAGGGAGAGGGCGGGGCACAGGGG |
| CCCGCATCCTCCCTCCCACCCCAACCAGCCCC | GGGGCTGGTTGGGGTGGGAGGGAGGATGCGGG |
| CCCGCCGGAGCCCCCCGGCCCCTCCGCGCCCC | GGGGCGCGGAGGGGCCGGGGGGCTCCGGCGGG |
| CCCGGGACCCCCCGCCCTGCCCCGCCGCCCCC | GGGGGCGGCGGGGCAGGGCGGGGGGTCCCGGG |
| CCCGGGGCGCCCTCCCGCGCCCTCTTGCACCC | GGGTGCAAGAGGGCGCGGGAGGGCGCCCCGGG |
| CCCTCATCAGCCCCTCGCCCCCTCCATGGCCC | GGGCCATGGAGGGGGCGAGGGGCTGATGAGGG |
| CCCTCCCTCCCCCCTCTGCCCCAGTGTGGCCC | GGGCCACACTGGGGCAGAGGGGGGAGGGAGGG |
| CCCTCTCCCACCCTAGCGTCCCCACCCTCCCC | GGGGAGGGTGGGGACGCTAGGGTGGGAGAGGG |
| CCCTGCCCCCCCACCCCCACCCACTGCTGCCC | GGGCAGCAGTGGGTGGGGGTGGGGGGGCAGGG |
| CCCTTCCCCACCCCCTCCCTCCCCGCAGGCCC | GGGCCTGCGGGGAGGGAGGGGGTGGGGAAGGG |
| GGGAAAGGGTGGGGGTTAGGGGAAGGTTGGGG | CCCCAACCTTCCCCTAACCCCCACCCTTTCCC |
| GGGAACGGGAGGGAACGAGAGGGAAGGGAGGG | CCCTCCCTTCCCTCTCGTTCCCTCCCGTTCCC |
| GGGAAGGACAGGGTAGGGTGGGGTGGGGTGGG | CCCACCCCACCCCACCCTACCCTGTCCTTCCC |
| GGGACGGGGTGGGAGGGGTGGGCCCTGGCGGG | CCCGCCAGGGCCCACCCCTCCCACCCCGTCCC |
| GGGCACCGGCGGGGAGGGCTGGGGGGCCCGGG | CCCGGGCCCCCCAGCCCTCCCCGCCGGTGCCC |
| GGGCGATGTGGGAAAGGAGGGGTGTTAAGGGG | CCCCTTAACACCCCTCCTTTCCCACATCGCCC |
| GGGCTCTGGGGGCGGCCAGGGGTGCCACTGGG | CCCAGTGGCACCCCTGGCCGCCCCCAGAGCCC |
| GGGCTGCGAGGGGGTGGTGAGGGTGACATGGG | CCCATGTCACCCTCACCACCCCCTCGCAGCCC |
| GGGGAAGGGAGGGGAGGGAGGGAGGGGGCGGG | CCCGCCCCCTCCCTCCCTCCCCTCCCTTCCCC |
| GGGGAGCCAGGGACCCCTGGGGGCCCCGTGGG | CCCACGGGGCCCCCAGGGGTCCCTGGCTCCCC |
| GGGGCGTACGGGTGGCCCCGGGGGGGCCGGGG | CCCCGGCCCCCCCGGGGCCACCCGTACGCCCC |
| GGGGCTGGGGGGAGGGGAGGGGGTGATGGGGG | CCCCCATCACCCCCTCCCCTCCCCCCAGCCCC |
| GGGGGAATGGGGGCAGGGTGGGTCTTCCAGGG | CCCTGGAAGACCCACCCTGCCCCCATTCCCCC |
| GGGGGCCCTTGGGAGGGATCGGGGGCCAAGGG | CCCTTGGCCCCCGATCCCTCCCAAGGGCCCCC |
| GGGGGGGCGGGGTGCGGGCGGGGTCCGGAGGG | CCCTCCGGACCCCGCCCGCACCCCGCCCCCCC |
| GGGGGTAATAGGGTGGAGGCGGGAGTGGGGGG | CCCCCCACTCCCGCCTCCACCCTATTACCCCC |
| GGGGTGGAGGGGGCCACCGGGGCAGGGAGGGG | CCCCTCCCTGCCCCGGTGGCCCCCTCCACCCC |
| GGGTGATGTGGGCCAAGCTGGGATGGGAGGGG | CCCCTCCCATCCCAGCTTGGCCCACATCACCC |
| GGGTGGCCGGGGCTGTGAGGGGCGGGCAGGGG | CCCCTGCCCGCCCCTCACAGCCCCGGCCACCC |
| CCCAGCCTAGCCCAACCCAGCCCAGCCCTGCCC | GGGCAGGGCTGGGCTGGGTTGGGCTAGGCTGGG |
| CCCATTCTCCCCCACTACTCCCCGCCCCGTCCC | GGGACGGGGCGGGGAGTAGTGGGGGAGAATGGG |
| CCCCACAATGCCCTCCCTCCCCCCACCCTCCCC | GGGGAGGGTGGGGGGAGGGAGGGCATTGTGGGG |
| CCCCACCCACCCCCTAGTGCCCCCAAGCATCCC | GGGATGCTTGGGGGCACTAGGGGGTGGGTGGGG |
| CCCCATCTTCCCCTGGTTGCCCCTCGGTTCCCC | GGGGAACCGAGGGGCAACCAGGGGAAGATGGGG |
| CCCCCCGCATCCCCCCCTTTCCCACCCGGCCCC | GGGGCCGGGTGGGAAAGGGGGGGATGCGGGGGG |
| CCCCGCCCGGCCCCGCCCGGCCCCGCCCCACCC | GGGTGGGGCGGGGCCGGGCGGGGCCGGGCGGGG |
| CCCCGGGCCACCCTGCTGGCCCCACCCAAGCCC | GGGCTTGGGTGGGGCCAGCAGGGTGGCCCGGGG |
| CCCCTTGCCCCCCAGCCCCGCCCCTCCACCCCC | GGGGGTGGAGGGGCGGGGCTGGGGGGCAAGGGG |
| CCCGCGGCTGCCCAGGGAGCCCCCGGGGCCCCC | GGGGGCCCCGGGGGCTCCCTGGGCAGCCGCGGG |
| CCCTCAACAGCCCAGCCCTCCCCTCGGGATCCC | GGGATCCCGAGGGGAGGGCTGGGCTGTTGAGGG |
| CCCTGAGGTCCCCGTTCTACCCCATCCCCACCC | GGGTGGGGATGGGGTAGAACGGGGACCTCAGGG |
| CCCTGGGATGCCCAGCCTCCCCCATTGATGCCC | GGGCATCAATGGGGGAGGCTGGGCATCCCAGGG |
| GGGATGGGATGGGATGGGATGGGATGGGATGGG | CCCATCCCATCCCATCCCATCCCATCCCATCCC |
| GGGCACCGCGGGGGCGGGCCGGGGGCGGCGGGG | CCCCGCCGCCCCCGGCCCGCCCCCGCGGTGCCC |
| GGGCCAGACTGGGGTGTGGGGGGCTGAGCTGGG | CCCAGCTCAGCCCCCCACACCCCAGTCTGGCCC |
| GGGCCGGGCCGGGCGTCCGAGGGTCTGGTCGGG | CCCGACCAGACCCTCGGACGCCCGGCCCGGCCC |
| GGGCGGTGCGGGGAGGGGAAGGGTGAGGAAGGG | CCCTTCCTCACCCTTCCCCTCCCCGCACCGCCC |
| GGGCTAGGCTGGGCTGGGCTGGGCTGGGGTGGG | CCCACCCCAGCCCAGCCCAGCCCAGCCTAGCCC |
| GGGGACCGCGGGGTGGGGGAGGGGGCAACAGGG | CCCTGTTGCCCCCTCCCCCACCCCGCGGTCCCC |
| GGGGCTCCGAGGGGACGGGAGGGGGGAGCAGGG | CCCTGCTCCCCCCTCCCGTCCCCTCGGAGCCCC |
| GGGGGCAGCTGGGGAGGGTGGGGATGGGAGGGG | CCCCTCCCATCCCCACCCTCCCCAGCTGCCCCC |
| GGGGGCCCCGGGGCTGCTGGGGGGGCACCAGGG | CCCTGGTGCCCCCCCAGCAGCCCCGGGGCCCCC |
| GGGGGTAGGAGGGGGCATGTGGGGAGGGCCGGG | CCCGGCCCTCCCCACATGCCCCCTCCTACCCCC |
| GGGGGTGCTCGGGTGAAGAGGGGGGACCCAGGG | CCCTGGGTCCCCCCTCTTCACCCGAGCACCCCC |
| GGGGTGGGAAGGGCTGGCCTGGGAAAAAAGGGG | CCCCTTTTTTCCCAGGCCAGCCCTTCCCACCCC |
| GGGTAGAAGAGGGTGGGTGTGGGATGGGGAGGG | CCCTCCCCATCCCACACCCACCCTCTTCTACCC |
| GGGTCCCGCTGGGGCGCGGGGGGCGCGGAGGGG | CCCCTCCGCGCCCCCCGCGCCCCAGCGGGACCC |
| GGGTGAGGCCGGGCCGGGCCGGGCCGGGTTGGG | CCCAACCCGGCCCGGCCCGGCCCGGCCTCACCC |

TABLE C

| | |
|---|---|
| GGGAGGGAGGGGGTGGG | CCCACCCCCTCCCTCCC |
| CCCCCCCTCCCGCTGCCC | GGGCAGCGGGAGGGGGGG |
| GGGAGGGAGAAGGGAGGG | CCCTCCCTTCTCCCTCCC |
| GGGGTAAGGGAGGGAGGG | CCCTCCCTCCCTTACCCC |
| CCCCTCCCTCCCCCATCCC | GGGATGGGGGAGGGAGGGG |
| GGGAGGGTGGGCTGTTGGG | CCCAACAGCCCACCCTCCC |
| GGGGAGGGCTGGGCTGGGG | CCCCAGCCCAGCCCTCCCC |
| CCCACCCTTTTCCCCTCCCC | GGGGAGGGGAAAAGGGTGGG |
| CCCTTCCCATGCCCCTTCCC | GGGAAGGGGCATGGGAAGGG |
| CCCTTCTCCCTCCCCTCCCC | GGGGAGGGGAGGGAGAAGGG |
| CCCTCGCCCATCCCACCGCCC | GGGCGGTGGGATGGGCGAGGG |
| GGGCAGGGCGCGGGAGGGGGG | CCCCCCTCCCCCGCCCTGCCC |
| GGGGGATGGGGTGGGAGGGGG | CCCCCTCCCACCCCATCCCCC |
| GGGTTGGGCGTGGGGGCTGGG | CCCAGCCCCCACGCCCAACCC |
| CCCACACCCCAGGCCCTGCCCC | GGGGCAGGGCCTGGGGTGTGGG |
| CCCATGGGCCCGCCCCACTCCC | GGGAGTGGGGCGGGCCCATGGG |
| CCCCTCCCATGGCCCTCCCCCC | GGGGGGAGGGCCATGGGAGGGG |
| GGGCTTGGGTGTTGGGACAGGG | CCCTGTCCCAACACCCAAGCCC |
| GGGGCAGTGCGTATGGGTTGGG | CCCAACCCATACCCACTGCCCC |
| GGGGTGTGGGTAGGGGAGTGGG | CCCACTCCCCTACCCACACCCC |
| CCCCGACCCTCGAACCCAGGCCC | GGGCCTGGGTTCGAGGGTCGGCG |
| CCCTCCCCCTCCCAGCCCTCCCC | GGGGAGGGCTGGGAGGGGGAGGG |
| CCCTCCCTTTCCCTCCCCCACCC | GGGTGGGGGAGGGAAAGGGAGGG |
| GGGAAGGGGCAGTCAGGGCTGGG | CCCAGCCCTGACTGCCCCTTCCC |
| GGGGTCGGGGTAAGGGGCAGGGG | CCCCTGCCCCTTACCCCGACCCC |
| GGGTCTGAGGGTGAGGGTGCGGG | CCCGCACCCTCACCCTCAGACCC |
| CCCAGCTCCCCTTCCCACTTGCCC | GGGCAAGTGGGAAGGGGAGCTGGG |
| CCCAGGCCCCTCCCACGATGACCC | GGGTCATCCTGGGAGGGGCCTGGG |
| CCCATCTCCCTTTCCACCCTCCCC | GGGGAGGGTGGAAAGGGAGATGGG |
| CCCGTGGTGCCCTCCCGGCTGCCC | GGGCAGCCGGGAGGGCACCACGGG |
| CCCTCCCTGTTCCCACCCACCCCC | GGGGGTGGGTGGGAACAGGGAGGG |
| GGGGAGGGGGAGGGAGCTGGGGGG | CCCCCCAGCTCCCTCCCCCTCCCC |
| GGGGGGAGTGGGAGGGAGGTGGGG | CCCCACCTCCCTCCCACTCCCCCC |
| CCCCTCACCCACCCCTGAAGATCCC | GGGATCTTCAGGGGTGGGTGAGGGG |
| CCCGCCCCCTCGGCTCCCCGCCCCC | GGGGGCGGGGAGCCGAGGGGGCGGG |
| CCCTCGCCCACCCCAGCCCCGCCCC | GGGGCGGGGCTGGGGTGGGCGAGGG |
| GGGCGGAGGGAAGGCGGGACGGGGG | CCCCCGTCCCGCCTTCCCTCCGCCC |
| GGGCTGGGACCAGGGGCTGCTGGGG | CCCCAGCAGCCCCTGGTCCCAGCCC |
| GGGGCGGGCAGGGCAGGGGCTGGGG | CCCCAGCCCCTGCCCTGCCCGCCCC |
| GGGGCGTCTAGGGGGTGGGGGTGGG | CCCACCCCCACCCCCTAGACGCCCC |
| CCCACAGGCCCCTCAGCCCCCCGCCC | GGGCGGGGGGCTGAGGGGCCTGTGGG |
| CCCCATGGTTCCCCGCCCCCACGCCC | GGGCGTGGGGGCGGGGAACCATGGGG |
| CCCCATTGCCCCACCCTCCCCTACCC | GGGTAGGGGAGGGTGGGGCAATGGGG |
| CCCCCGCCCCTTCCCCGCCAGCCCCC | GGGGGCTGGCGGGGAAGGGGCGGGGG |
| CCCTCCCAGCCGGCCCCACTGCCCCC | GGGGGCAGTGGGGCCGGCTGGGAGGG |
| GGGGAACCGAATGCAGGGTTGCTGGG | CCCAGCAACCCTCCATTCCCTTCCCC |
| GGGGAGGGACGGGCTTGGCGGAGGGG | CCCCTCCCCCAAGCCCGTCCCTCCCC |
| GGGGCCCAGGGCCTGCTGGGATGGGG | CCCCATCCCAGCAGGCCCTGGGCCCC |
| GGGGTGGGGAATAGGGTGGGAGTGGG | CCCACTCCCACCCTATTCCCCACCCC |
| GGGTAGGGGAGGGTGGGGCAATGGGG | CCCCATTGCCCCACCCTCCCCTACCC |
| CCCCTGCCCCGTTCCCCCACCCAGCCC | GGGCTGGGTGGGGGAACGGGGCAGGGG |
| CCCGCCACCCCCGCACCCATACAACCC | GGGTTGTATGGGTGCCGGGGTGGCGGG |
| CCCTCCCTCCCACCCATCCCCCATCCC | GGGATGGGGGATGGGTGGGAGGGAGGG |
| CCCTGCTCTCCCTTCTCCCCTTCTCCC | GGGAGAAGGGGAGAAGGGAGAGCAGGG |
| GGGAAAGTTCGGGGGGAGGGGGGAGGG | CCCTCCCCCCTCCCCCCGAACTTTCCC |
| GGGAGGGCTGGCTTGGGCTGAGAAGGG | CCCTTCTCAGCCCAAGCCAGCCCTCCC |
| GGGAGTTGGGGTGGGAAGGGGTATGGG | CCCATACCCCTTCCCACCCCAACTCCC |
| GGGGAGGGGAATGGGAGGGGAAATGGG | CCCATTTCCCCTCCCATTCCCCTCCCC |
| CCCCATCCCTCTTTCCCCTCCCACTCCC | GGGAGTCGGAGGGCAAAGAGGGATGGGG |
| CCCCCTCCCCACAACCCCTCCCACCCCC | GGGGGTGGGAGGGGTTGTGGGGAGGGGC |
| CCCCCTCCCCCTCCCACCCCCAGTCCCC | GGGGACTGGGGGTGGGAGGGGGAGGGGG |
| CCCCCTCCCCTCCCTCCCGCCCGCCCCC | GGGGGCGGGCGGGAGGGAGGGGAGGGGG |
| CCCGCCCCCGCCCTGGCCCCCAGTGCCC | GGGCACTGGGGGCCAGGGCGGGGGCGGG |
| CCCTACCGCCCAAAGCCCGCCTCCACCC | GGGTGGAGGCGGGCTTTGGGCGGTAGGG |
| GGGAGAGAGGGCTGGGGGCAAGGGTGGG | CCCACCCTTGCCCCCAGCCCTCTCTCCC |
| GGGCATAGTGGGGCCGAGGGATCATGGG | CCCATGATCCCTCGGCCCCACTATGCCC |
| GGGGAGGGGGGCGAGGGGACGGGGCGGG | CCCGCCCCGTCCCCTCGCCCCCCTCCCC |
| GGGGATGGAGGGGAGGGGGGAGGGGGGG | CCCCCCCTCCCCCCTCCCCTCCATCCCC |
| CCCATCTCCCTTTCTCCCCTCCCCATCCC | GGGATGGGGAGGGGAGAAAGGGAGATGGG |
| CCCGCCCCCCGCCCCAGCCCACTCGGCCC | GGGCCGAGTGGGCTGGGGCGGGGGGCGGG |
| GGGGAGGACTGGGGCGCTCGGGAGAGGGG | CCCCTCTCCCGAGCGCCCCAGTCCTCCCC |
| GGGGCCGGTGGGCCCTGGGGACCCATGGG | CCCATGGGTCCCCAGGGCCCACCGGCCCC |
| GGGGTGGTGGGCGTCGGGGGCGAGGAGGG | CCCTCCTCGCCCCCGACGCCCACCACCCC |
| CCCCAAGCCCCCGAAGCCCCCTAAGCCCCC | GGGGGCTTAGGGGGCTTCGGGGGCTTGGGG |
| CCCTCCCATCCCCAATTCCCTGTTCCCCCC | GGGGGGAACAGGGAATTGGGGATGGGAGGG |
| CCCTCTCCACCCACAGGCCCCGCACCTCCC | GGGAGGTGCGGGGCCTGTGGGTGGACAGGG |
| GGGAAGGGGGAGATTTGGGGGAGGGAGGGG | CCCCTCCCTCCCCCAAATCTCCCCCTTCCC |
| GGGAATGGGAGGGGCAAAGGGAAGGATGGG | CCCATCCTTCCCTTTGCCCCTCCCATTCCC |
| GGGGAAGCAGGGGAGGTGGGAGGGCTTGGG | CCCAAGCCCTCCCACCTCCCCTGCTTCCCC |
| GGGGGCTTAGGGGGTTTTGGGGGCTTGGGG | CCCCAAGCCCCCAAAACCCCCTAAGCCCCC |
| GGGTCCCGGGGCGCCGGTGGGCTGAAATGGG | CCCATTTCAGCCCACCGGCGCCCCGGGACCC |
| CCCATGTCGCCCTTCCTGTCCCTGCACCACCC | GGGTGGTGCAGGGACAGGAAGGGCGACATGGG |
| CCCCCAGCTCCCCGCTGAGCCCCCCGGTGTCCC | GGGACACCGGGGGGCTCAGCGGGGAGCTGGGGG |
| GGGTAGATCTGGGGTTGGGCGGGCGGCGCCGGG | CCCGGCGCCGCCCGCCCAACCCCAGATCTACCC |

### Example 3

### Representative Embodiments

Provided hereafter are examples of representative embodiments.

1. A method for identifying a molecule that binds to a nucleic acid, which comprises

contacting a nucleic acid and a compound that binds to the nucleic acid with a test molecule, wherein the nucleic acid comprises a nucleotide sequence containing (a) one or more nucleotide sequences of Table A, (b) a complement of (a), (c) an RNA nucleotide sequence encoded by (a), (d) an RNA nucleotide sequence encoded by (b), or (e) a substantially identical variant nucleotide sequence of the foregoing; and

detecting the amount of the compound bound or not bound to the nucleic acid,

whereby the test molecule is identified as a molecule that binds to the nucleic acid containing the human nucleotide sequence when less of the compound binds to the nucleic acid in the presence of the test molecule than in the absence of the test molecule.

2. The method of aspect 1, wherein the compound is in association with a detectable label.

3. The method of aspect 1 or 2, wherein the compound is radiolabled.

4. The method of any one of aspects 1-3, wherein the compound is a quinolone or a porphyrin.

5. The method of any one of aspects 1-3, wherein the nucleic acid is in association with a solid phase.

6. The method of any one of aspects 1-5, wherein the test molecule is a quinolone derivative.

7. The method of aspect 6, wherein the quinolone derivative is a compound of Tables 1A-1C, Table 2, Table 3 or Table 4.

8. The method of any one of aspects 1-7, wherein the nucleotide sequence is a DNA nucleotide sequence.

9. The method of any one of aspects 1-7, wherein the nucleotide sequence is a RNA nucleotide sequence.

10. The method of any one of aspects 1-7, wherein the nucleic acid comprises one or more nucleotide analogs or derivatives.

11. A method for identifying a molecule that causes displacement of a protein from a nucleic acid, which comprises

contacting a nucleic acid containing a human nucleotide sequence and a protein with a test molecule, wherein the nucleic acid is capable of binding to the protein and the nucleotide sequence comprises (a) one or more nucleotide sequences of Table A, (b) a complement of (a), (c) an RNA nucleotide sequence encoded by (a), (d) an RNA nucleotide sequence encoded by (b), or (e) a substantially identical variant nucleotide sequence of the foregoing; and

detecting the amount of the nucleic acid bound or not bound to the protein,

whereby the test molecule is identified as a molecule that causes protein displacement when less of the nucleic acid binds to the protein in the presence of the test molecule than in the absence of the test molecule.

12. The method of aspect 11, wherein the protein is in association with a detectable label.

13. The method of aspect 11, wherein the protein is in association with a solid phase.

14. The method of aspect 11, wherein the nucleic acid is in association with a detectable label.

15. The method of aspect 11, wherein the nucleic acid is in association with a solid phase.

16. The method of any one of aspects 11-15, wherein the test molecule is a quinolone derivative.

17. The method of aspect 16, wherein the quinolone derivative is a compound of Tables 1A-1C, Table 2, Table 3 or Table 4.

18. The method of any one of aspects 11-17, wherein the nucleotide sequence is a DNA nucleotide sequence.

19. The method of any one of aspects 11-17, wherein the nucleotide sequence is a RNA nucleotide sequence.

20. The method of any one of aspects 11-17, wherein the nucleic acid comprises one or more nucleotide analogs or derivatives.

21. A method of identifying a modulator of nucleic acid synthesis, which comprises:

contacting a template nucleic acid, a primer oligonucleotide having a nucleotide sequence complementary to a template nucleic acid nucleotide sequence, extension nucleotides, a polymerase and a test molecule under conditions that allow the primer oligonucleotide to hybridize to the template nucleic acid, wherein the template nucleic acid comprises (a) one or more nucleotide sequences of Table A, (b) a complement of (a), (c) an RNA nucleotide sequence encoded by (a), (d) an RNA nucleotide sequence encoded by (b), or (e) a substantially identical variant nucleotide sequence of the foregoing; and

detecting the presence, absence or amount of an elongated primer product synthesized by extension of the primer nucleic acid,

whereby the test molecule is identified as a modulator of nucleic acid synthesis when a different amount of an elongated primer product is synthesized in the presence of the test molecule than in the absence of the test molecule.

22. The method of aspect 21, wherein the template nucleic acid is DNA.

23. The method of aspect 21, wherein the template nucleic acid RNA.

24. The method of aspect 21, 22 or 23, wherein the polymerase is a DNA polymerase.

25. The method of aspect 21, 22 or 23, wherein the polymerase is an RNA polymerase.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

What is claimed is:

1. A method for identifying a molecule that binds to a nucleic acid containing a human nucleotide sequence, which method comprises
contacting the nucleic acid and a compound that binds to the nucleic acid with a test molecule, wherein the nucleic acid comprises a nucleotide sequence containing (a) one or more nucleotide sequences of Table A, (b) a complement of (a), (c) an RNA nucleotide sequence encoded by (a), (d) an RNA nucleotide sequence encoded by (b), or (e) a substantially identical variant nucleotide sequence of the foregoing; and
detecting the amount of the compound bound or not bound to the nucleic acid,
whereby the test molecule is identified as a molecule that binds to the nucleic acid containing the human nucleotide sequence when less of the compound binds to the nucleic acid in the presence of the test molecule than in the absence of the test molecule.

2. The method of claim 1, wherein the compound is in association with a detectable label.

3. The method of claim 1 or 2, wherein the compound is radiolabeled.

4. The method of any one of claims 1-3, wherein the compound is a quinolone or a porphyrin.

5. The method of any one of claims 1-3, wherein the nucleic acid is in association with a solid phase.

6. The method of any one of any of claims 1-5, wherein the test molecule is a quinolone derivative.

7. The method of claim 6, wherein the quinolone derivative is a compound of Tables 1A-1C, Table 2, Table 3 or Table 4.

8. The method of any one of any of claims 1-7, wherein the nucleotide sequence is a DNA nucleotide sequence.

9. The method of any one of any of claims 1-7, wherein the nucleotide sequence is a RNA nucleotide sequence.

10. The method of any one of claims 1-7, wherein the nucleic acid comprises one or more nucleotide analogs or derivatives.

11. A method for identifying a molecule that causes displacement of a protein from a nucleic acid, which method comprises
contacting a protein and a nucleic acid containing a human nucleotide sequence with a test molecule, wherein the nucleic acid is capable of binding to the protein and the nucleotide sequence of the nucleic acid comprises (a) one or more nucleotide sequences of Table A, (b) a complement of (a), (c) an RNA nucleotide sequence encoded by (a), (d) an RNA nucleotide sequence encoded by (b), or (e) a substantially identical variant nucleotide sequence of the foregoing; and
detecting the amount of the nucleic acid bound or not bound to the protein,
whereby the test molecule is identified as a molecule that causes protein displacement when less of the nucleic acid binds to the protein in the presence of the test molecule than in the absence of the test molecule.

12. The method of claim 11, wherein the protein is in association with a detectable label.

13. The method of claim 11, wherein the protein is in association with a solid phase.

14. The method of claim 11, wherein the nucleic acid is in association with a detectable label.

15. The method of claim 11, wherein the nucleic acid is in association with a solid phase.

16. The method of any one of claims 11-15, wherein the test molecule is a quinolone derivative.

17. The method of claim 16, wherein the quinolone derivative is a compound of Tables 1A-1C, Table 2, Table 3 or Table 4.

18. The method of any one of claims 11-17, wherein the nucleotide sequence is a DNA nucleotide sequence.

19. The method of any one of claims 11-17, wherein the nucleotide sequence is a RNA nucleotide sequence.

20. The method of any one of claims 11-17, wherein the nucleic acid comprises one or more nucleotide analogs or derivatives.

21. A method of identifying a modulator of nucleic acid synthesis, which method comprises:
contacting a template nucleic acid, a primer oligonucleotide having a nucleotide sequence complementary to a template nucleic acid nucleotide sequence, extension nucleotides, a polymerase and a test molecule under conditions that allow the primer oligonucleotide to hybridize to the template nucleic acid, wherein the template nucleic acid comprises (a) one or more nucleotide sequences of Table A, (b) a complement of (a), (c) an RNA nucleotide sequence encoded by (a), (d) an RNA nucleotide sequence encoded by (b), or (e) a substantially identical variant nucleotide sequence of the foregoing; and
detecting the presence, absence or amount of an elongated primer product synthesized by extension of the primer nucleic acid,
whereby the test molecule is identified as a modulator of nucleic acid synthesis when a different amount of an elongated primer product is synthesized in the presence of the test molecule than in the absence of the test molecule.

22. The method of claim 21, wherein the template nucleic acid is DNA.

23. The method of claim 21, wherein the template nucleic acid RNA.

24. The method of claim 21 or 22, wherein the polymerase is a DNA polymerase.

25. The method of claim 21 or 23, wherein the polymerase is an RNA polymerase.
